(19) United States
(12) Patent Application Publication
(10) Pub. No.: US 2026/0137819 A1
(43) Pub. Date: May 21, 2026

(54) FOLLICLE-STIMULATING HORMONE RECEPTOR (FSHR) TARGETED THERAPEUTICS AND USES THEREOF

(71) Applicant: Radionetics Oncology, Inc., San Diego, CA (US)

(72) Inventors: Jian ZHAO, San Diego, CA (US); Mi CHEN, San Diego, CA (US); Yunfei ZHU, San Diego, CA (US); Yifeng XIONG, San Diego, CA (US); Junjie LIU, San Diego, CA (US)

(21) Appl. No.: 19/116,036

(22) PCT Filed: Oct. 3, 2023
(86) PCT No.: PCT/US2023/075860

§ 371 Date: Mar. 27, 2025

Related U.S. Application Data
(60) Provisional application No. 63/378,484, filed on Oct. 5, 2022.

(51) Int. Cl. A61K 51/04; A61K 9/00; A61P 35/00

(52) U.S. Cl. CPC A61K 51/0497; A61K 9/0019; A61K 51/0453; A61K 51/0463; A61P 35/00; A61K 2121/00; A61K 2123/00

(57) ABSTRACT

Described herein are radiotherapeutics that target tumor cells expressing the follicle-stimulating hormone receptor (FSHR) and their use in the treatment and/or diagnosis of cancer.

### CROSS-REFERENCE TO RELATED APPLICATIONS

This application claims the benefit of U.S. Provisional Patent Application No. 63/378,484, filed Oct. 5, 2022, which is incorporated herein by reference in its entirety.

### FIELD OF THE INVENTION

Described herein are radiotherapeutics that target tumor cells expressing the follicle-stimulating hormone receptor (FSHR) and methods of using such radiotherapeutics as cancer therapeutics, diagnostics, or both.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g. adenomas). Malignant neoplasms display features such as genetic mutations, loss of normal function, rapid division, and ability metastasize (invade) to other tissues; and neoplasms of uncertain or unknown behavior. Malignant neoplasms (i.e., cancerous solid tumors) are the leading cause of death in industrialized countries. Noncancerous neoplasms including benign adenomas can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy.

As one of 3 main classes of cell surface receptors, G protein-coupled receptors (GPCRs) are frequently overexpressed in tumor cells and are considered promising targets for selective tumor therapy. FSHR is a GPCR that is primarily expressed in the ovaries and testes. Targeted delivery of radionuclides to tumors with small molecule FSHR-targeting ligands offers a novel approach to treat and diagnose various cancers, including, but not limited to ovarian cancer, prostate cancer, breast cancer, testicular cancer, lung cancer, liver adenocarcinoma, colon adenocarcinoma, stomach adenocarcinoma, kidney cancer or bladder cancer.

### SUMMARY OF THE INVENTION

Described herein are radiopharmaceuticals for use in the diagnosis and/or treatment of tumors. The present disclosure provides an alternative and improved method for the treatment of tumors by targeting tumors that overexpress the follicle-stimulating hormone receptor (FSHR). In some embodiments, the radiopharmaceuticals disclosed herein are useful in the treatment of tumors that overexpress FSHR. In some other embodiments, the radiopharmaceuticals disclosed herein are useful in the identification of tissues or organs in a subject comprising tumors overexpressing FSHR. The radiopharmaceuticals disclosed herein are also useful in vivo imaging of a subject for the presence of and distribution of tumors that overexpress FSHR in the subject.

In one aspect, described herein is a compound of Formula (A), or a pharmaceutically acceptable salt thereof,

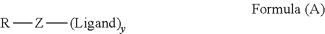

wherein:
R is -L^{A}-L^{B}-R6, -L^{A}_(L^{B}-R6)₂, or -L^{A}-(L^{B}-R6) 3,
L^{A} is a linker or is absent;
L^{B} is a linker or is absent;
R⁶ is a chelating moiety or a radionuclide complex thereof; is C₁-C₆ alkylene, C₁-C₆ alkylene-O—, —O—C₁-C₆ alkylene-, —C(═O)NR¹⁰—, —NR¹⁰C. (—O)—, —NR¹⁰—, —O—, —S—, —S(═O)—, —SO₂—, —NR¹⁰C. (═O)NR¹⁰—, or —CR10═NO—;
each R10 is independently H or unsubstituted C₁-C₄ alkyl;
Ligand is a small molecule modulator of the follicle-stimulating hormone receptor (FSHR); and
y is 1, 2 or 3.

In some embodiments, R is -L^{B}-R6. In some embodiments, Ligand is a small molecule agonist of FSHR. Ligand is a small molecule antagonist of FSHR. In some embodiments, Ligand comprises a thiazolidinone (TZD), a diketopiperazine, a hexahydroquinoline, a thienopyrimidine, a piperidine carboxyamide, an acyltryptophanol, a pyrrolobenzodiazepine, an aminoalkylamide, an isoxazolyl-thiazolyl, a dihydropyrrolo[2,1-a]isoquinoline, a dihydroimidazo[5,1-A]isoquinoline, a dihydrobenzoindazole, a fused tricylic imidazole, a fused tricylic pyrazole, a 1,4-dihydrochromeno[4,3-c]pyrazole, or a dihydro-1H-benzo[g]indole. In some embodiments, Ligand comprises a dihydropyrrolo[2,1-a]isoquinoline, a dihydroimidazo[5,1-A]isoquinoline, a dihydrobenzoindazole, a 1,4-dihydrochromeno[4,3-c]pyrazole, or a dihydro-1H-benzo[g]indole. In some embodiments, Ligand comprises a naphthalene sulfonic acid, a (bis) sulfonic acid, a (bis)benzamide, a tetrahydroquinoline (THQ), a benzamide, a naphthalene sulfonic acid, or a tetrahydroquinoline. In some embodiments, the fused tricylic imidazole is a dihydrobenzo-imidazole. In some embodiments, the fused tricylic imidazole is a dihydrobenzo-pyrazole.

In another aspect, described herein is a compound of Formula (B), or a pharmaceutically acceptable salt thereof:

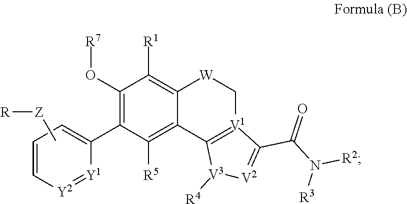

wherein:
R¹ is H, F, Cl, Br or I;
W is O or —CH₂—;
V¹ is C or N;
V² is C, CR⁸ or N;
V³ is CR⁸ or N;
R² and R³ are each independently substituted or unsubstituted alkyl;
or R² and R³ with the nitrogen to which they are connected form a substituted or unsubstituted 5 to 8-membered heterocycloalkyl;
R⁴ is a substituted or unsubstituted aryl or substituted or unsubstituted heteroaryl;
R⁵ is H, F, Cl, Br or I;
Y¹ and Y² are each independently CR⁹ or N;
Z is C₁-C₆ alkylene, —C(═O)NR¹⁰—, —NR¹⁰C(═O)—, —NR¹⁰—, —O—, —S—, —S(═O)—, —SO₂—, —NR¹⁰C. (═O)NR¹⁰, or —CR¹⁰═NO—;
R is -L¹-R⁶, —CH-(L¹-R⁶)₂, substituted or unsubstituted C₁-C₁₀alkylene-CH-(L¹-R⁶)₂, substituted or unsubstituted 2 to 10 membered heteroalkylene-CH-(L¹-R⁶)₂, —N-(L¹-R⁶)₂, substituted or unsubstituted C₁-C₁₀alkylene-N(L¹-R⁶)₂, substituted or unsubstituted 2 to 10 membered heteroalkylene-N(L¹-R⁶)₂, or —(CH₂CH₂O)_{q}—CH₂CH₂N(L¹-R⁶)₂—;
q is 1, 2, 3, 4, 5 or 6;
L¹ is a linker or is absent;
R⁶ is a chelating moiety or a radionuclide complex thereof;
R⁷ is independently H or substituted or unsubstituted alkyl;
each R⁸ is independently H, —N(R¹¹)₂, F, Cl, Br, I, or —OR¹¹;
each R⁹ is independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —N(R¹²)₂, or —OR¹²;
each R¹⁰ is independently H or unsubstituted C₁-C₄ alkyl;
each R¹¹ is independently H or unsubstituted C₁-C₄ alkyl; and
each R¹² is independently H or unsubstituted C₁-C₄ alkyl.

In some embodiments, the compound of Formula (B) has the following structure, or a pharmaceutically acceptable salt thereof:

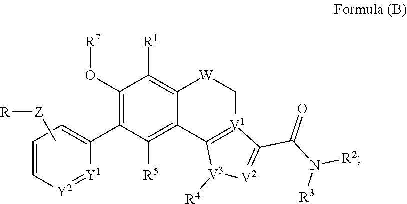

wherein R² and R³ with the nitrogen to which they are connected form a substituted or unsubstituted 5 to 8-membered heterocycloalkyl, wherein the substituted heterocycloalkyl is substituted with one or more R^{2a} and one or more R^{2b}, wherein each R^{2a} and R^{2b} are independently H, halogen, substituted or unsubstituted C₁-C₄ alkyl, substituted or unsubstituted C₁-C₄ fluoroalkyl, substituted or unsubstituted 2 to 6 membered heteroalkyl, —C(═O) R^{2c}, —CN, —NH2, or —OH; wherein R^{2c} is substituted or unsubstituted C₁-C₄ alkyl or substituted or unsubstituted C₃-C₆ cycloalkyl. In some embodiments, the heterocycloalkyl is substituted with one R^{2a} and one R^{2b}. In some embodiments, R²a and R^{2b} are each —CH₃. In some embodiments, R²a is H and R^{2b} is —C(═O) R^{2c}, wherein R^{2c} is CH₃, CH₂CH₃, cyclopropyl, cyclobutyl, cyclopentyl or cyclohexyl. In some embodiments, R^{2b} is

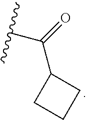 [SMILES: *C(=O)C1CCC1]

In some embodiments, the compound of Formula (B) has the following structure, or a pharmaceutically acceptable salt thereof:

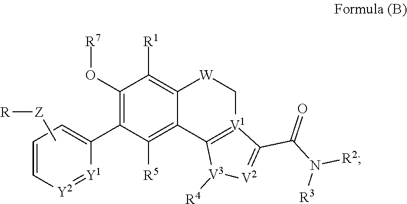

wherein R⁴ is a substituted or unsubstituted aryl or substituted or unsubstituted heteroaryl; wherein the substituted aryl is substituted with one R^{4a}, one R^{4b}, and one R^{4c}, wherein R^{4a}, R^{4b}, and R^{4c} are each independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —NH₂, or —OH; and wherein the substituted heteroaryl is substituted with one R^{4d} and one Rde, wherein R^{4d} and R⁴ e are each independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —NH₂, or —OH. In some embodiments, R^{4a} and R^{4b} are each independently F, Cl, Br, I or —CH₃ and R⁴ is H. In some embodiments, R^{4d} and R^{4e} are H.

In another aspect, described herein is a compound of Formula (I), or a pharmaceutically acceptable salt thereof:

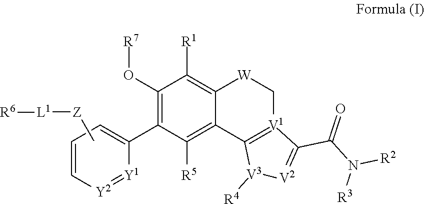

wherein:
R¹ is H, F, Cl, Br or I;
W is O or —CH₂—:
V¹ is C or N;
V² is C, CR⁸ or N;
V³ is CR⁸ or N;
R² and R³ are each independently substituted or unsubstituted alkyl;
or R² and R³ with the nitrogen to which they are connected form a substituted or unsubstituted 5 to 8-membered heterocycloalkyl;
R⁴ is a substituted or unsubstituted aryl or substituted or unsubstituted heteroaryl;
R⁵ is H, F, Cl, Br or I;
Y¹ and Y² are each independently CR⁹ or N;
Z is C₁-C₆ alkylene, —C(═O)NR¹⁰—, —NR¹⁰° C. (═O)—, —NR¹⁰—, —O—, —S—, —S(═O)—, —SO₂—, —NR¹⁰C(═O)NR¹⁰—, or —CR¹⁰—NO—;
L¹ is a linker or is absent;
R⁶ is a chelating moiety or a radionuclide complex thereof;
R⁷ is H or substituted or unsubstituted alkyl;
each R⁸ is H, —N(R¹¹)₂, F, Cl, Br, I, or —OR¹¹;
each R⁹ is H, halogen, substituted or unsubstituted C₁-C₄ alkyl, substituted or unsubstituted C₁-C₄ fluoroalkyl, substituted or unsubstituted 2 to 6 membered heteroalkyl, —CN, —N(R¹²)₂, or —OR¹²;
   each R¹⁰ is independently H or unsubstituted C₁-C₄ alkyl;
   each R¹¹ is independently H or unsubstituted C₁-C₄ alkyl; and
   each R¹² is independently H or unsubstituted C₁-C₄ alkyl.

In some embodiments, the compound of Formula (I) has the following structure, or a pharmaceutically acceptable salt thereof:

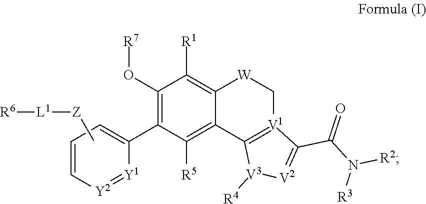

wherein R² and R³ with the nitrogen to which they are connected form a substituted or unsubstituted 5 to 8-membered heterocycloalkyl, wherein the substituted heterocycloalkyl is substituted with one or more R^{2a} and one or more R^{2b}, wherein each R^{2a} and R^{2b} are independently H, halogen, substituted or unsubstituted C₁-C₄ alkyl, substituted or unsubstituted C₁-C₄ fluoroalkyl, substituted or unsubstituted 2 to 6 membered heteroalkyl, —C(═O) R^{2c}, —CN, —NH2, or —OH; wherein R^{2c} is substituted or unsubstituted C₁-C₄ alkyl or substituted or unsubstituted C₃-C₆ cycloalkyl. In some embodiments, the heterocycloalkyl is substituted with one R^{2a} and one R^{2b}. In some embodiments, R^{2a} and R^{2b} are each —CH₃. In some embodiments, R^{2a} is H and R^{2b} is —C(═O) R^{2c}, wherein R^{2c} is CH₃, CH₂CH₃, cyclopropyl, cyclobutyl, cyclopentyl or cyclohexyl. In some embodiments, R^{2b} is

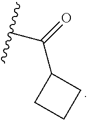 [SMILES: *C(=O)C1CCC1]

In some embodiments, the compound of Formula (I) has the following structure, or a pharmaceutically acceptable salt thereof:

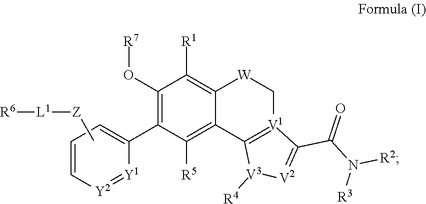

wherein R⁴ is a substituted or unsubstituted aryl or substituted or unsubstituted heteroaryl;
wherein the substituted aryl is substituted with one R^{4a}, one R^{4b}, and one R^{4c}, wherein R^{4a}, R^{4b}, and R^{4c} are each independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —NH₂, or —OH; and wherein the substituted heteroaryl is substituted with one R^{4d} and one R^{4e}, wherein R^{4d} and R^{4e} are each independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —NH₂, or —OH. In some embodiments, R^{4a} and R^{4b} are each independently F, Cl, Br, I or —CH₃ and R^{4c} is H. In some embodiments, R^{4d} and R^{4e} are H.

In some embodiments, the compound of Formula (I) has the structure of Formula (Ia), or a pharmaceutically acceptable salt thereof:

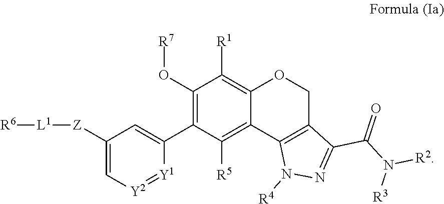

In some embodiments, the compound of Formula (I) has the structure of Formula (Ib), or a pharmaceutically acceptable salt thereof:

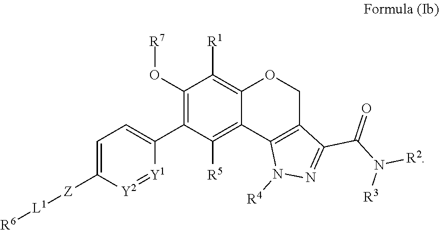

In some embodiments, the compound of Formula (I) has the structure of Formula (Ic), or a pharmaceutically acceptable salt thereof:

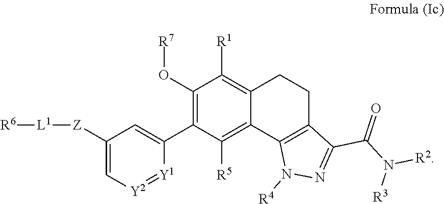

In some embodiments, the compound of Formula (I) has the structure of Formula (Id), or a pharmaceutically acceptable salt thereof:

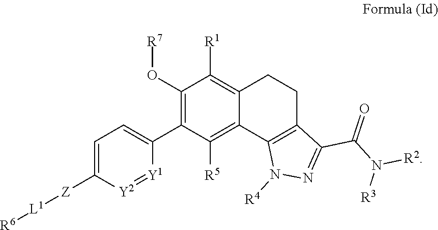

In some embodiments, the compound of Formula (I) has one of the following structures, or a pharmaceutically acceptable salt thereof:

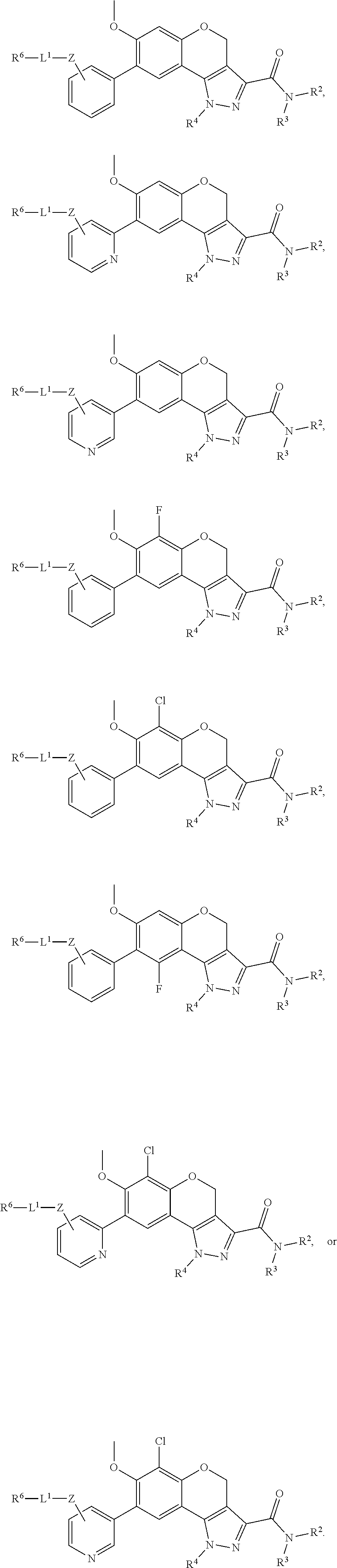

In some embodiments, R⁶ is a chelating moiety selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6″-(((pyridine-2,6-diylbis(methylene))bis(azanetriyl))-tetrakis(methylene))-tetrapicolinic acid (H₄py4pa); Hapy4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′-(1,10-dioxa-4,7, 13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

In some embodiments, R⁶ is a chelating moiety selected from the group consisting of:

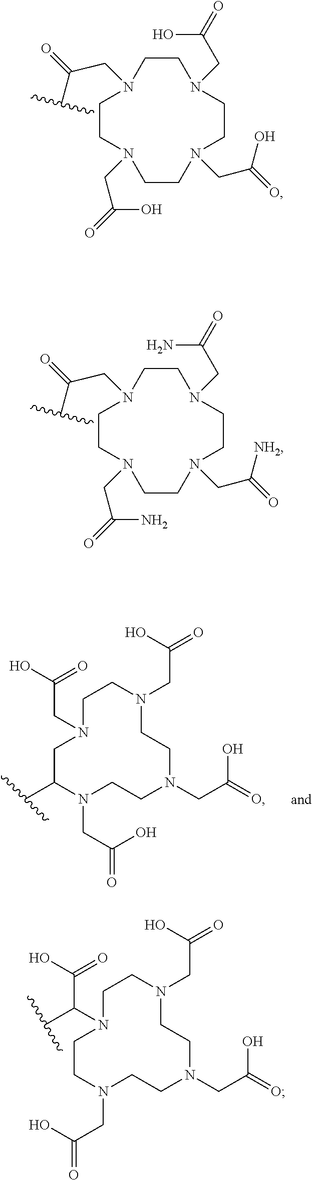 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]

or a radionuclide complex thereof.

In some embodiments, L¹ is -L²-, -L³-, -L⁴-, -L⁵-, -L⁶-, -L⁷-, -L²-L³-, -L²-L⁴-, -L²-L⁷-, -L⁵-L⁶-L⁷-; L² is absent, substituted or unsubstituted C₁-C20alkylene, substituted or unsubstituted C₁-C₂₀alkylene-NR¹³—, substituted or unsubstituted C₁-C₂₀alkylene-C(═O)—, substituted or unsubstituted C₁-C20alkylene-C(═O)NR¹³—, substituted or unsubstituted C₁-C₂₀alkylene-NR¹³C(═O)—, substituted or unsubstituted 2 to 20 membered heteroalkylene, —(CH₂CH₂O)_{z}—, (OCH₂CH₂)_{z}—, —(CH₂CH₂O)_{w}—CH₂CH₂—, —CH₂CH₂NR¹³—(CH₂CH₂O)_{w}—, —(CH₂CH₂O)_{w}—CH₂CH₂NR¹³—, —CH₂CH₂NR¹³C(═O)—(CH₂CH₂O)_{w}—, —(CH₂CH₂O)_{w}—CH₂CH₂NR¹³C(═O)—, CH₂CH₂C(═O)NR¹³—; w is 1, 2, 3, 4, 5, or 6; z is 1, 2, 3, 4, 5, 6, 7, 8, 9, or 10; L³ is absent, a natural or unnatural amino acid or peptide that is formed from two or more independently selected natural and unnatural amino acids, wherein when two or more amino acids are present then the N atom of the amide linking the amino acids is optionally substituted with C₁-C₆ alkyl; L⁴ is absent, substituted or unsubstituted 2 to 10 membered heteroalkylene, —CH₂—(OCH₂CH₂)ᵥ—, —(CH₂CH₂O)ᵥ—CH₂CH₂—, —(CH₂CH₂O)ₜCH₂CH₂NR¹+C(═O)(CH₂CH₂O)ᵤCH₂CH₂—, —(CH₂CH₂O)ᵤCH₂CH₂C(═O)NR¹ₜ(CH₂CH₂O)ᵥCH₂CH₂—, —C(═O)CH₂CH₂, —CH₂CH₂C(═O)—, or C₁-C₆ alkylene that is optionally substituted with 1 or 2 groups independently selected from —OH, —NR¹⁴₂, —CO₂R¹⁴, —O(CH₂CH₂O)ᵤ—CH₃, —NR¹⁴(CH₂CH₂O)ᵤ—CH₃, —NR¹+C(═O)(CH₂CH₂O)ᵤ—CH₃, or —CH₂OCH₂CH₂CO₂R¹⁴; v is 1, 2, 3, 4, 5, or 6; u is 1, 2, 3, 4, 5, or 6; L⁵ is absent, —O—, —S—, —S(═O)—, —S(═O)₂—, —NR¹⁵—, —CH(═NH)—, —CH(═N—NH)—, —CCH₃(═NH)—, —CCH₃ (═N—NH)—, —C(═O)NR¹⁵—, —NR¹⁵C(═O), —NR¹⁵C(═O)O—, —NR¹⁵C(═O)NR¹⁵—, or —OC(═O)NR¹⁵—; L⁶ is absent or -L⁸-L⁹-L¹⁰; L⁸ is absent, —(CH₂)ₜ—, —NR^{w}—, —NR^{w}—(CH₂)ₜ—, —(CH₂)ₜ—C(═O)—, —C(═O)—(CH₂)—, —(CH₂)ₜ—NR^{w}—, —(CH₂)—NR^{w}C(═O)—, —(CH₂)—C(═O)NR^{w}—, —CH(NHR^{w})—(CH₂)ᵥ—C(—O)—, —NR^{w}C(═O)—(CH₂)ₜ—, and —C(═O)NR^{w}—(CH₂)ₜ—; t is 0, 1, 2, or 3; L¹⁰ is absent, —(CH₂)ᵣ—, —NR^{w}—, —NR^{w}—(CH₂)ᵣ—, —(CH₂)—C(═O)—, —C(═O)—(CH₂)ᵣ—, —(CH₂), —NR^{w}—, —(CH₂)—NR^{w}C(═O)—, —(CH₂)—C(═O)NR^{w}—, —CH(NHR^{w})—(CH₂)—C(═O)—, —NR^{w}C(═O)—(CH₂)ᵣ—, and —C(═O)NR^{w}—(CH₂)ᵥ—; r is 0, 1, 2, or 3; each R^{w} is independently selected from H, C₁-C₆ alkyl, C₁-C₆ alkyl-CO₂H, (CH₂CH₂O)ₛ—CH₃, —C(—O)—(CH₂CH₂O)ₛ—CH₃, or —(CH₂CH₂O)ₛ—CH₂CH₂CO₂H; s is 1, 2, 3, 4, 5, or 6; L⁹ is substituted or unsubstituted cycloalkylene, substituted or unsubstituted heterocycloalkylene, substituted or unsubstituted arylene, or substituted or unsubstituted heteroarylene; each R¹³, R¹⁴ and R¹⁵ are independently selected from H or unsubstituted C₁-C₄ alkyl; and L⁷ is absent, —NH—, —N(CH₃)—, —O—NH—, or substituted or unsubstituted N-heterocycloalkylene, or —O—NH═ (substituted or unsubstituted N-heterocycloalkylene).

In some embodiments, the radionuclide of the radionuclide complex is a lanthanide or an actinide. In some embodiments, the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium. In some embodiments, the radionuclide of the radionuclide complex is a diagnostic or therapeutic radionuclide. In some embodiments, the radionuclide of the radionuclide complex is an Auger electron-emitting radionuclide, α-emitting radionuclide, β-emitting radionuclide, or γ-emitting radionuclide. In some embodiments, the radionuclide of the radionuclide complex is 111-indium (¹¹¹ In), 115-indium (¹¹⁵ In), 67-gallium (⁶⁷Ga), 68-gallium (⁶⁸Ga), 70-gallium (⁷⁰Ga), 225-actinium (²²⁵ Ac), 175-lutetium (¹⁷⁵Lu) or 177-lutetium (¹⁷⁷Lu).

Also described herein is a pharmaceutical composition comprising a compound described herein (e.g., a compound of Formula (I), Formula (B) or Formula (A)), or a pharmaceutically acceptable salt thereof, and at least one pharmaceutically acceptable excipient. In some embodiments, the pharmaceutical composition is formulated for administration to a mammal by intravenous administration or subcutaneous administration. In some embodiments, the pharmaceutical composition is formulated for administration to a mammal by intravenous administration.

In another aspect, described herein is a method for the treatment of cancer comprising administering to a mammal with cancer an effective amount of a compound described herein (e.g., a compound of Formula (I), Formula (B) or Formula (A)), or a pharmaceutically acceptable salt thereof, or an effective amount of pharmaceutical composition comprising a compound described herein (e.g., a compound of Formula (I), Formula (B) or Formula (A)), or a pharmaceutically acceptable salt thereof. In some embodiments, the cancer comprises tumors and the tumor overexpress the follicle-stimulating hormone receptor (FSHR). In some embodiments, the cancer is ovarian cancer, prostate cancer, breast cancer, testicular cancer, lung cancer, liver adenocarcinoma, colon adenocarcinoma, stomach adenocarcinoma, kidney cancer or bladder cancer. In some embodiments, the cancer is ovarian cancer. In some embodiments, the cancer is prostate cancer. In some embodiments, the cancer is breast cancer. In some embodiments, the cancer is testicular cancer. In some embodiments, the cancer is lung cancer. In some embodiments, the cancer is liver adenocarcinoma. In some embodiments, the cancer is colon adenocarcinoma. In some embodiments, the cancer is stomach adenocarcinoma. In some embodiments, the cancer is kidney cancer. In some embodiments, the cancer is bladder cancer.

In another aspect, described herein is a method for treating tumors in a mammal with a radionuclide comprising administering to the mammal a compound described herein (e.g., a compound of Formula (I), Formula (B) or Formula (A)), or a pharmaceutically acceptable salt thereof, or a pharmaceutical composition comprising a compound described herein (e.g., a compound of Formula (I), Formula (B) or Formula (A)), or a pharmaceutically acceptable salt thereof. In some embodiments, the mammal has been diagnosed with ovarian cancer. In some embodiments, the mammal has been diagnosed with prostate cancer, breast cancer or testicular cancer.

In another aspect, described herein is a method of targeting delivery of a radionuclide to tumors in a mammal comprising administering to a mammal with tumors a compound described herein (e.g., a compound of Formula (I), Formula (B) or Formula (A)), or a pharmaceutically acceptable salt thereof, or a pharmaceutical composition comprising a compound described herein (e.g., a compound of Formula (I), Formula (B) or Formula (A)), or a pharmaceutically acceptable salt thereof; wherein the tumors overexpress the follicle-stimulating hormone receptor (FSHR).

In another aspect, described herein is a method for identifying tissues or organs in a mammal with tumors expressing the follicle-stimulating hormone receptor (FSHR) comprising administering to the mammal a compound described herein (e.g., a compound of Formula (I), Formula (B) or Formula (A)), or a pharmaceutically acceptable salt thereof, or a pharmaceutical composition comprising a compound described herein (e.g., a compound of Formula (I), Formula (B) or Formula (A)), or a pharmaceutically acceptable salt thereof; and performing positron emission tomography (PET) analysis, single-photon emission computerized tomography (SPECT), or magnetic resonance imaging (MRI); wherein R⁶ is a chelating moiety-diagnostic radionuclide complex.

In yet another aspect, described herein is a method for the in vivo imaging of tissues or organs in a mammal with tumors expressing the follicle-stimulating hormone receptor (FSHR) comprising administering to the mammal a compound described herein (e.g., a compound of Formula (I), Formula (B) or Formula (A)), or a pharmaceutically acceptable salt thereof, or a pharmaceutical composition comprising a compound described herein (e.g., a compound of Formula (I), Formula (B) or Formula (A)), or a pharmaceutically acceptable salt thereof; and performing positron emission tomography (PET) analysis, single-photon emission computerized tomography (SPECT), or magnetic resonance imaging (MRI); wherein R⁶ is a chelating moiety-diagnostic radionuclide complex.

In any of the embodiments disclosed herein, the mammal is a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Cancer, a disease in which some cells undergo a genetic change in the control of their growth and replication that results in uncontrolled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias (cancers of bone marrow), and lymphomas and myelomas (cancers of the immune system). Neoplasms are abnormal growth of cells that result in solid tumors which may be benign (i.e. do not display malignant features and are generally unlikely to become dangerous such as adenomas), malignant (i.e. display features such as genetic mutations, loss of normal function, rapid division, and ability metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often requires multiple interventions as well as combination with radiation and chemotherapy. Chemotherapy proves to be a potent weapon in the fight against cancer in many cases. However, further optimization is required. Chemotherapy is typically performed by systemic administration of potent cytotoxic drugs, but these compounds lack tumor selectivity and therefore also kill healthy cells in the body. The resulting non-specific toxicity is the cause of severe side effects of chemotherapy which does not target the cancerous cells specifically over other cells. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Like chemotherapy, many radiation therapy options also lack tumor cell identification properties needed to achieve the ultimate goal of targeted tumor therapy with drug molecules or radionuclides.

Described herein are radiopharmaceuticals that selectively deliver radionuclides to the malignant cells that overexpress FSHR.

Follicle-stimulating hormone (FSH) is a central hormone in mammalian reproductive biology. It promotes the mature spermatogenesis for men and follicular maturation for women, respectively. FSH receptor (FSHR) is a glycosylated transmembrane protein belonging to the family of G-protein-coupled receptors (GPCR). In adult humans and animals, low levels of FSHR are expressed only in endothelial cells of ovary and testis. Immunostaining showed that FSHR was overexpressed in the vasculatures of various solid tumors including prostate, breast, lung, and ovarian cancer. On the contrary, healthy and nonmalignant inflammatory tissues were always FSHR negative. These findings imply that the receptor may be a target for cancer detection, image guided cancer surgery, and selective tumor killing by a radionuclide conjugate.

GPCRs are generally poorly antigenic making them difficult targets for antibody-based strategies. The large size of antibodies can impact homogenous uptake and they may be unable to penetrate deep in solid tumors. Additionally, antibodies may present difficulties during production, including inter-batch variability.

Peptides are intrinsically sensitive to proteolytic enzymes and peptidases present in most tissues may rapidly degrade the peptides into multiple fragments which no longer have significant affinity to the intended receptors. In addition, peptides may cause unwanted immunogenic responses complicating later stages of development by masking the therapeutic effect and impacting the safety assessment.

When peptide ligands are linked to radionuclide payloads, the resulting conjugates often degrade apart rapidly in blood plasma and produce cytotoxic or radioactive peptide fragments which may nonspecifically bind to both tumor and normal tissue. Such premature breakdown of peptide radionuclide conjugates reduce the amount of radionuclide payloads distributed to targeted tumors, lowering treatment efficacy, and possibly increasing toxicity. In addition, peptides are most likely exclusively excreted via kidney, which may limit their applications. Marked kidney uptake of some peptide-based therapeutics has limited their routine use.

High affinity, small molecule ligands that bind GPCRs have been described and are cell permeable and can access populations of receptors in endoplasmic reticulum and endosomes. Owing to the low molecular weight of small molecules, vascular permeability and tumor penetration should be improved compared to high molecular weight conjugates based on peptides and antibodies. The affinity of small molecule ligands in many cases surpasses that of FDA approved antibodies by orders of magnitude.

### The Follicle-Stimulation Hormone Receptor (FSHR)

The follicle-stimulating hormone receptor (FSHR) is a G-protein-coupled receptor that is expressed on ovaries and testis. FSHR is a glycosylated transmembrane protein that presents a portion that is extracellular. This region recognizes and binds to its endogenous hormone, follicle-stimulating hormone (FSH). FSH, a key hormone in mammalian reproduction, is produced mainly in the anterior pituitary gland, and the target organs are the receptors on the ovaries and testis. In females, FSH stimulates follicular maturation and estrogen production through aromatization of androgens. In males, FSH stimulates Sertoli-cell proliferation in immature testes and maintains normal spermatogenesis in adults.

In adult humans and animals, FSHR is known to be expressed only in the testicular Sertoli cells and the ovarian granulosa cells, and it is expressed in low levels in the endothelial cells of the ovary and testis. In the testis, FSHR mediates the translocation of FSH across the blood-testis endothelial barrier by a process of receptor-mediated transcytosis.

### Ovarian Cancer

Ovarian cancers are histologically and genetically divided into type I or type II. Type I cancers are of low histological grade and include endometrioid, mucinous, and clear-cell carcinomas. Type II cancers are of higher histological grade and include serous carcinoma and carcinosarcoma.

The most common type of ovarian cancer is epithelial ovarian carcinoma, comprising more than 95% of cases. There are five main subtypes of ovarian carcinoma, of which high-grade serous carcinoma (HGSC) is the most common. The other subtypes of ovarian carcinoma include low-grade serous carcinoma, clear cell carcinoma, endometrioid carcinoma and mucinous carcinoma. Less common types of ovarian cancer include germ cell tumors and sex cord stromal tumors.

Ovarian cancer is staged using the FIGO staging system and uses information obtained after surgery, which can include a total abdominal hysterectomy via midline laparotomy, removal of (usually) both ovaries and Fallopian tubes, (usually) the omentum, pelvic (peritoneal) washings, assessment of retroperitoneal lymph nodes (including the pelvic and para-aortic lymph nodes), appendectomy in suspected mucinous tumors, and pelvic/peritoneal biopsies for cytopathology. Around 30% of ovarian cancers that appear confined to the ovary have metastasized microscopically, which is why even stage-I cancers must be resected completely. 22% of cancers presumed to be stage I are observed to have lymphatic metastases. The AJCC (American Joint Committee on Cancer) stage is the same as the FIGO stage. The AJCC staging system describes the extent of the primary tumor (T), the absence or presence of metastasis to nearby lymph nodes (N), and the absence or presence of distant metastasis(M). The most common stage at diagnosis is stage IIIc, with over 70% of diagnoses.

Tumors can form in the ovaries. The types of treatment used to treat ovarian tumors include: surgery, radiation therapy, and chemotherapy. Surgery to remove the ovaries (oophorectomy) and fallopian tubes (salpingectomy or both (salpingo-oophorectomy) is often used to treat ovarian cancer. Sometimes surgery is done to remove the nearby lymph nodes and other tissue where the cancer has spread. Radiation therapy uses high-energy x-rays or other types of radiation to kill cancer cells or keep them from growing. There are two types of radiation therapy: external radiation therapy uses a machine outside the body to send radiation toward the area of the body with cancer; internal radiation therapy uses a radioactive substance sealed in needles, seeds, wires, or catheters that are placed directly into or near the cancer. Additionally, targeted radiopharmaceuticals can provide targeted radiation to the site of the tumor. Chemotherapy is a cancer treatment that uses drugs to stop the growth of cancer cells, either by killing the cells or by stopping them from dividing.

Thus, a need exists for treatment options for ovarian tumors, such as epithelial ovarian tumors. Described herein are radiopharmaceuticals that target delivery of radionuclides to ovarian tumors, which overexpress the FSHR. Targeted therapies usually cause less harm to normal cells than chemotherapy or radiation therapy do.

### Prostate Cancer

Prostate cancer is cancer of the prostate, which is the gland in the male reproductive system that surrounds the urethra, just below the bladder. Prostate cancer is one of the most common types of cancer. Almost all prostate cancers are adenocarcinomas, which develop from the glandular epithelial cells (the cells that make the prostate fluid that is added to the semen). While very rare, other types of cancer that can start in the prostate include: small cell carcinomas, neuroendocrine tumors (other than small cell carcinomas), transitional cell carcinomas, and sarcomas. Many prostate cancers grow slowly and are confined to the prostate gland, where they may not cause serious harm. However, while some types of prostate cancer grow slowly and may need minimal or no treatment, other types are aggressive and can spread quickly.

Prostate cancer is staged using the TNM system developed by the AJCC. The three key components of the TNM system, include: T (tumor), which describes the tumor's size, location and how deep it has grown into the tissue; N (node), which indicates whether cancer cells have spread to nearby lymph nodes or the channels connecting the lymph nodes; and M (metastasis), which refers to whether the cancer cells have spread to distant organs or tissue. Prostate cancer stages range from 1 through 4.

Tumors can form in the prostate and treatment recommendations may vary widely. Low-grade prostate cancer may not need treatment right away. For some, treatment may never be needed. Instead, their doctors may recommend observation (sometimes called watchful waiting) or active surveillance. Surgery is a common choice to try to cure prostate cancer if it is not thought to have spread outside the prostate gland. The main type of surgery for prostate cancer is a radical prostatectomy. In this operation, the surgeon removes the entire prostate gland plus some of the tissue around it, including the seminal vesicles. Other treatment options for prostate cancer include: radiation therapy, cryotherapy, hormone therapy, chemotherapy, immunotherapy, and targeted therapy. Radiation therapy uses high-energy x-rays or other types of radiation to kill cancer cells or keep them from growing. Chemotherapy is a cancer treatment that uses drugs to stop the growth of cancer cells, either by killing the cells or by stopping them from dividing.

Thus, a need exists for treatment options for prostate tumors, such as epithelial prostate tumors. Described herein are radiopharmaceuticals that target delivery of radionuclides to prostate tumors, which overexpress the FSHR. Targeted therapies usually cause less harm to normal cells than chemotherapy or radiation therapy do.

### Testicular Cancer

Testicular cancer occurs in the testicles (testes), which are located inside the scrotum, a loose bag of skin underneath the penis. The testicles produce male sex hormones and sperm for reproduction. Compared with other types of cancer, testicular cancer is rare. But testicular cancer is the most common cancer in American males between the ages of 15 and 35.

Types of testicular cancer include germ cell tumors, intratubular germ cell neoplasia (carcinoma in situ of the testicle (CIS)), and stromal tumors.

The main types of germ cell tumors are seminomas and non-seminomas. Seminomas tend to grow and spread more slowly than non-seminomas. The 2 main sub-types of seminona tumors are classical (or typical) seminomas and spermatocytic seminomas. Non-seminoma tumors usually occur in men between their late teens and early 30s. The 4 main types of non-seminoma tumors are embryonal carcinoma, yolk sac carcinoma, choriocarcinoma, and teratoma. Most tumors are a mix of different types (sometimes with seminoma cells too), but this doesn't change the treatment of most non-seminoma cancers.

Gonadal stromal tumors can start in the supportive and hormone-producing tissues, or stroma, of the testicles. Gonadal stromal tumors make up less than 5% of adult testicular tumors, but up to 20% of childhood testicular tumors. The main types are Leydig cell tumors and Sertoli cell tumors. Most Leydig and Sertoli tumors are benign.

The stages of testicular cancer are indicated by Roman numerals that range from 0 to III, with the lowest stages indicating cancer that is limited to the area around the testicle. Depending on the type and stage of testicular cancer, you may receive one of several treatments, or a combination. Treatments for testicular cancer include surgery, radiation therapy, chemotherapy and high-dose chemotherapy and stem cell transplant.

Thus, a need exists for treatment options for testicular tumors, such as embryonal carcinoma. Described herein are radiopharmaceuticals that target delivery of radionuclides to testicular tumors, which overexpress the FSHR. Targeted therapies usually cause less harm to normal cells than chemotherapy or radiation therapy do.

Solid Tumors: Benign and/or Malignant Neoplasms (Cancer)

In one aspect, compounds of Formula (I), Formula (B) or Formula (A) are used to treat benign and/or malignant neoplasms (solid tumors), wherein the neoplasm comprises cells that overexpress FSHR on the cell surface.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

In some embodiments, compounds of Formula (I), Formula (B) or Formula (A) are used to treat an adenoma. An adenoma is a tumor that is not cancer. It starts in gland -like cells of the epithelial tissue (thin layer of tissue that covers organs, glands, and other structures within the body). An adenoma can grow from many glandular organs, including the adrenal glands, pituitary gland, thyroid, prostate, and others Even though benign, they have the potential to cause serious health complications by compressing other structures (mass effect) and by producing large amounts of hormones in an unregulated, non-feedback-dependent manner (causing paraneoplastic syndromes). Over time adenomas may transform to become malignant, at which point they are called adenocarcinomas.

Adenomas may be found in the colon (e.g. adenomatous polyps, which have a tendency to become malignant and to lead to colon cancer), kidneys (e.g. renal adenomas may be precursor lesions to renal carcinomas), adrenal glands (e.g. adrenal adenomas; some secrete hormones such as cortisol, causing Cushing's syndrome, aldosterone causing Conn's syndrome, or androgens causing hyperandrogenism), thyroid (e.g. thyroid adenoma), pituitary (e.g. pituitary adenomas, such as prolactinoma, Cushing's disease and acromegaly), parathyroid (e.g. an adenoma of a parathyroid gland may secrete inappropriately high amounts of parathyroid hormone and thereby cause primary hyperparathyroidism), liver (e.g. hepatocellular adenoma), breast (e.g. fibroadenomas), appendix (e.g. cystadenoma), bronchial (e.g. bronchial adenomas may cause carcinoid syndrome, a type of paraneoplastic syndrome), prostate (e.g. prostate adenoma), sebaceous gland (e.g. sebaceous adenoma), and salivary glands.

Metastasis is the spread of malignant cells to new areas of the body, often by way of the lymph system or bloodstream. A metastatic tumor is one that has spread from the primary site of origin, or where it started, into different areas of the body. Metastatic tumors comprise malignant cells that may express cell surface FSHR.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing FSHR, wherein the tumor is a primary or metastatic tumor. In some embodiments, the tumor to be treated comprises tumor cells expressing FSHR, wherein the tumor is a primary or metastatic tumor of ovarian origin. In some embodiments, the tumor to be treated comprises tumor cells expressing FSHR, wherein the tumor is a primary or metastatic tumor of prostate origin. In some embodiments, the tumor to be treated comprises tumor cells expressing FSHR, wherein the tumor is a primary or metastatic tumor of testicular origin.

In some embodiments, compounds of Formula (I), Formula (A) or Formula (B) are used to treat a carcinoma. Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

In some embodiments, compounds of Formula (I), Formula (A) or Formula (B) are used to treat a sarcoma. Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Primary and metastatic tumors include, e.g., lung cancer (including, but not limited to, lung adenocarcinoma, squamous cell carcinoma, large cell carcinoma, bronchioloalveolar carcinoma, non-small-cell carcinoma, small cell carcinoma, mesothelioma); breast cancer (including, but not limited to, ductal carcinoma, lobular carcinoma, inflammatory breast cancer, clear cell carcinoma, mucinous carcinoma); colorectal cancer (including, but not limited to, colon cancer, rectal cancer); anal cancer; pancreatic cancer (including, but not limited to, pancreatic adenocarcinoma, islet cell carcinoma, neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinoma and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinoma, gastrointestinal stromal tumor); multiple myeloma; testicular cancer; germ cell tumor; neuroendocrine tumor; cervical cancer; carcinoids of the gastrointestinal tract, breast, and other organs; and signet ring cell carcinoma.

### Representative Follicle-Stimulating Hormone Receptor (FSHR) Targeting Ligands

In some embodiments, the compound of Formula (I), Formula (B) or Formula (A) has an affinity to FSHR that is at least 10-fold, at least 50-fold, at least 100-fold, at least 200-fold, at least 500-fold, or at least 1000-fold greater than the affinity for other non-target receptors. In some embodiments, the compound of Formula (I), Formula (B) or Formula (A) is selective for FSHR as compared to any one of the other glycoprotein hormone receptors, including luteinizing hormone (LH), thyroid-stimulating hormone (TSH) and human chorionic gonadotropin (hCG). In some embodiments, the compound of Formula (I), Formula (B) or Formula (A) has an affinity to FSHR that is at least 10-fold, at least 50-fold, at least 100-fold, at least 200-fold, at least 500-fold, or at least 1000-fold greater than the affinity for any one of LH, TSH and hCG.

In some embodiments, the compound of Formula (I), Formula (B) or Formula (A) preferentially accumulates in tumor tissues that express the targeted FSHR. In some embodiments, the compound of Formula (I), Formula (B) or Formula (A) preferentially accumulates in tissues or organs comprising tumor cells that express FSHR as compared to tissues or organ(s) lacking tumor cells that express FSHR. In some embodiments, the compound of Formula (I), Formula (B) or Formula (A) preferentially accumulates at least 1-fold, at least 2-fold, 3-fold, at least 4-fold, at least 5-fold, or greater than 5-fold more in tissues or organ(s) comprising tumor cells that express FSHR as compared to tissues or organs lacking tumor cells that express FSHR. In some embodiments, the compound of Formula (I), Formula (B) or Formula (A) preferentially accumulates in tissues or organs comprising tumor cells that express FSHR as compared to healthy tissues or organ(s) lacking tumor cells that express FSHR in lower levels. In some embodiments, the compound of Formula (I), Formula (B) or Formula (A) preferentially accumulates at least 1-fold, at least 2-fold, 3-fold, at least 4-fold, at least 5-fold, or greater than 5-fold more in tissues or organ(s) comprising tumor cells that express FSHR as compared to healthy tissues or organs lacking tumor cells that express FSHR in lower levels. It is understood that the compound may accumulate in certain tissues and organs involved in the metabolism and/or excretion of therapeutics, including but not limited to the kidneys and liver.

In one aspect, the FSHR targeting ligand is a compound of Formula (A), or a pharmaceutically acceptable salt thereof:

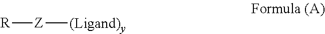

wherein:
R is -L^{A}-L^{B}-R⁶, -L^{A}-(L^{B}-R⁶)₂, or -L^{A}-(L^{B}-R⁶) 3,
L^{A} is a linker or is absent;
L^{B} is a linker or is absent;
R⁶ is a chelating moiety or a radionuclide complex thereof;
Z is C₁-C₆ alkylene, C₁-C₆ alkylene-O—, —O—C₁-C₆ alkylene-, —C(═O)NR¹⁰—, —NR¹⁰° C. (═O)—, —NR¹⁰—, —O—, —S—, —S(═O)—, —SO₂—, —NR¹⁰C. (═O)NR¹⁰—, or —CR¹⁰—NO—;
each R¹⁰ is independently H or unsubstituted C₁-C₄ alkyl;
Ligand is a small molecule modulator of the follicle-stimulating hormone receptor (FSHR); and
y is 1, 2 or 3.

In some embodiments, R is -L^{B}-R⁶.

In some embodiments, Ligand is a small molecule agonist of FSHR. In some embodiments, Ligand is a small molecule antagonist of FSHR.

In some embodiments, Ligand comprises a thiazolidinone (TZD), a diketopiperazine, a hexahydroquinoline, a thienopyrimidine, a piperidine carboxyamide, an acyltryptophanol, a pyrrolobenzodiazepine, an aminoalkylamide, an isoxazolyl-thiazolyl, a dihydropyrrolo[2,1-alisoquinoline, a dihydroimidazo[5,1-A]isoquinoline, a dihydrobenzoindazole, a fused tricylic imidazole, a fused tricylic pyrazole, a 1,4-dihydrochromeno[4,3-c]pyrazole, or a dihydro-1H-benzo[g]indole. In some embodiments, Ligand comprises a dihydropyrrolo[2,1-a]isoquinoline, a dihydroimidazo[5,1-A]isoquinoline, a dihydrobenzoindazole, a 1,4-dihydrochromeno[4,3-c]pyrazole, or a dihydro-1H-benzo[g]indole. In some embodiments, Ligand comprises a naphthalene sulfonic acid, a (bis) sulfonic acid, a (bis)benzamide, a tetrahydroquinoline (THQ), a benzamide, a naphthalene sulfonic acid, or a tetrahydroquinoline. In some embodiments, the fused tricylic imidazole is a dihydrobenzo-imidazole. In some embodiments, the fused tricylic imidazole is a dihydrobenzo-pyrazole.

In some embodiments, the compound has the structure of Formula (B), or a pharmaceutically acceptable salt thereof. In some embodiments, described herein is a compound of Formula (B), or a pharmaceutically acceptable salt thereof:

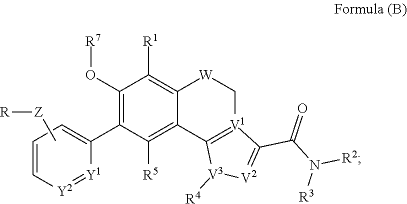

wherein:
R¹ is H, F, Cl, Br or I;
W is O or —CH₂—:
V¹ is C or N;
V² is C, CR⁸ or N;
V³ is CR⁸ or N;
R² and R³ are each independently substituted or unsubstituted alkyl;
or R² and R³ with the nitrogen to which they are connected form a substituted or unsubstituted 5 to 8-membered heterocycloalkyl;
R⁴ is a substituted or unsubstituted aryl or substituted or unsubstituted heteroaryl;
R⁵ is H, F, Cl, Br or I;
Y¹ and Y² are each independently CR⁹ or N;
Z is C₁-C₆ alkylene, —C(═O)NR¹⁰—, —NR¹⁰° C. (═O)—, —NR¹⁰—, —O—, —S—, —S(═O)—, —SO₂—, —NR¹⁰ C(═O)NR¹⁰—, or —CR¹⁰—NO—;
R is -L¹-R⁶, —CH-(L¹-R⁶)₂, substituted or unsubstituted C₁-C₁₀alkylene-CH-(L¹-R⁶)₂, substituted or unsubstituted 2 to 10 membered heteroalkylene-CH-(L¹-R⁶)₂, —N-(L¹-R⁶)₂, substituted or unsubstituted C₁-C₁₀alkylene-N(L¹-R⁶)₂, substituted or unsubstituted 2 to 10 membered heteroalkylene-N(L¹-R⁶)₂, or —(CH₂CH₂O) q-CH₂CH₂N(L¹-R⁶) 2;
q is 1, 2, 3, 4, 5 or 6;
L¹ is a linker or is absent;
R⁶ is a chelating moiety or a radionuclide complex thereof;
R⁷ is H or substituted or unsubstituted alkyl;
each R⁸ is independently H, —N(R¹¹)₂, F, Cl, Br, I, or —OR¹¹;
each R⁹ is independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —N(R¹²)₂, or —OR¹²;
each R¹⁰ is independently H or unsubstituted C₁-C₄ alkyl;
each R¹¹ is independently H or unsubstituted C₁-C₄ alkyl; and
each R¹² is independently H or unsubstituted C₁-C₄ alkyl.

In some embodiments, the compound has the structure of Formula (I), or a pharmaceutically acceptable salt thereof:

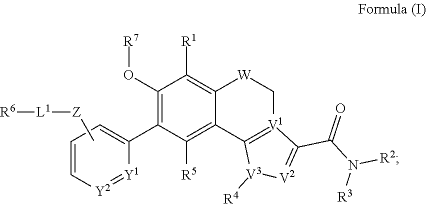

wherein:
R¹ is H, F, Cl, Br or I;
W is O or —CH₂—:
V¹ is C or N;
V² is C, CR⁸ or N;
V³ is CR⁸ or N;
R² and R³ are each independently substituted or unsubstituted alkyl;
or R² and R³ with the nitrogen to which they are connected form a substituted or unsubstituted 5 to 8-membered heterocycloalkyl;
R⁴ is a substituted or unsubstituted aryl or substituted or unsubstituted heteroaryl;
R⁵ is H, F, Cl, Br or I;
Y¹ and Y² are each independently CR⁹ or N;
Z is C₁-C₆ alkylene, —C(═O)NR¹⁰, —NR¹⁰C(═O)—, —NR¹⁰—, —O—, —S—, —S(═O)—, —SO₂—, —NR¹⁰C(═O)NR¹⁰—, or —CR¹⁰═NO—;
L¹ is a linker or is absent;
R⁶ is a chelating moiety or a radionuclide complex thereof;
R⁷ is H or substituted or unsubstituted alkyl;
each R⁸ is independently H, —N(R¹¹)₂, F, Cl, Br, I, or —OR¹¹;
each R⁹ is independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —N(R¹²)₂, or —OR¹²;
each R¹⁰ is independently H or unsubstituted C₁-C₄ alkyl;
each R¹¹ is independently H or unsubstituted C₁-C₄ alkyl; and
each R¹² is independently H or unsubstituted C₁-C₄ alkyl.

In some embodiments, the compound of Formula (I) has the structure of Formula (Ia), or a pharmaceutically acceptable salt thereof:

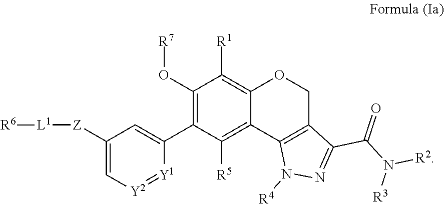

In some embodiments, the compound of Formula (I) has the structure of Formula (Ib), or a pharmaceutically acceptable salt thereof:

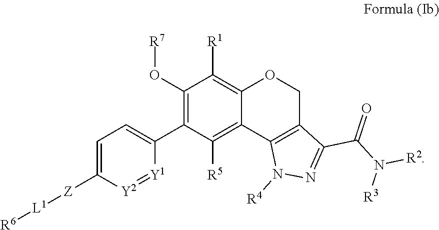

In some embodiments, the compound of Formula (I) has the structure of Formula (Ic), or a pharmaceutically acceptable salt thereof:

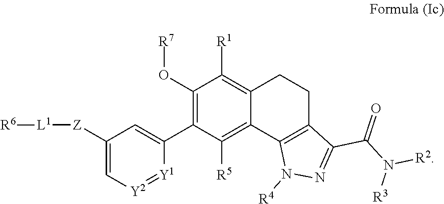

In some embodiments, the compound of Formula (I) has the structure of Formula (Id), or a pharmaceutically acceptable salt thereof:

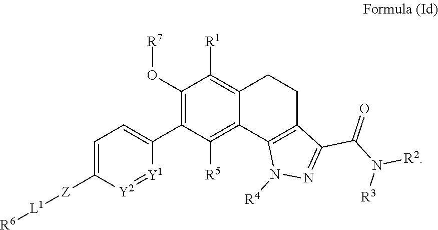

In some embodiments, R¹ is H, F or Cl. In some embodiments, R¹ is H or F. In some embodiments, R¹ is H. In some embodiments, R¹ is F. In some embodiments, R¹ is Cl. In some embodiments, R¹ is Br. In some embodiments, R¹ is I.

In some embodiments, R⁵ is H or F. In some embodiments, R⁵ is H. In some embodiments, R⁵ is F. In some embodiments, R⁵ is C₁. In some embodiments, R⁵ is Br. In some embodiments, R⁵ is I.

In some embodiments, Y¹ and Y² are CR⁹. In some embodiments, Y¹ is N and Y² is CR⁹. In some embodiments, Y¹ is CR⁹ and Y² is N. In some embodiments, Y¹ is N and Y² is N. In some embodiments, Y¹ and Y² are CH. In some embodiments, Y¹ is N and Y² is CH. In some embodiments, Y¹ is CH and Y² is N. In some embodiments, Y¹ is N and Y² is N.

In some embodiments, R⁷ is C₁-C₄ alkyl. In some embodiments, R⁷ is —CH₃. In some embodiments, R⁷ is —CH₂CH₃. In some embodiments, R⁷ is H.

In some embodiments, each R⁹ is independently H, halogen, —CH₃, or —CF3. In some embodiments, R⁹ is H. In some embodiments, R⁹ is halogen. In some embodiments, R⁹ is F. In some embodiments, R⁹ is C₁. In some embodiments, R⁹ is Br. In some embodiments, R⁹ is I. In some embodiments, R⁹ is unsubstituted C₁-C₄ alkyl. In some embodiments, R⁹ is —CH₃. In some embodiments, R⁹ is —CF3. In some embodiments, R⁹ is substituted or unsubstituted 2 to 6 membered heteroalkyl. In some embodiments, R⁹ is —CN. In some embodiments, R⁹ is —N(R¹²)₂. In some embodiments, R⁹ is —NH₂. In some embodiments, R⁹ is —OR¹². In some embodiments, R⁹ is —OH.

In some embodiments, R¹⁰ is H. In some embodiments, R¹⁰ is —CH₃.

In some embodiments, R¹¹ is H. In some embodiments, R¹¹ is —CH₃.

In some embodiments, R¹² is H. In some embodiments, R¹² is —CH₃.

In some embodiments, R¹³ is H. In some embodiments, R¹³ is —CH₃.

In some embodiments, R¹⁴ is H. In some embodiments, R¹⁴ is —CH₃.

In some embodiments, R¹⁵ is H. In some embodiments, R¹⁵ is —CH₃.

In some embodiments, the compound of Formula (I) has one of the following structures, or a pharmaceutically acceptable salt thereof:

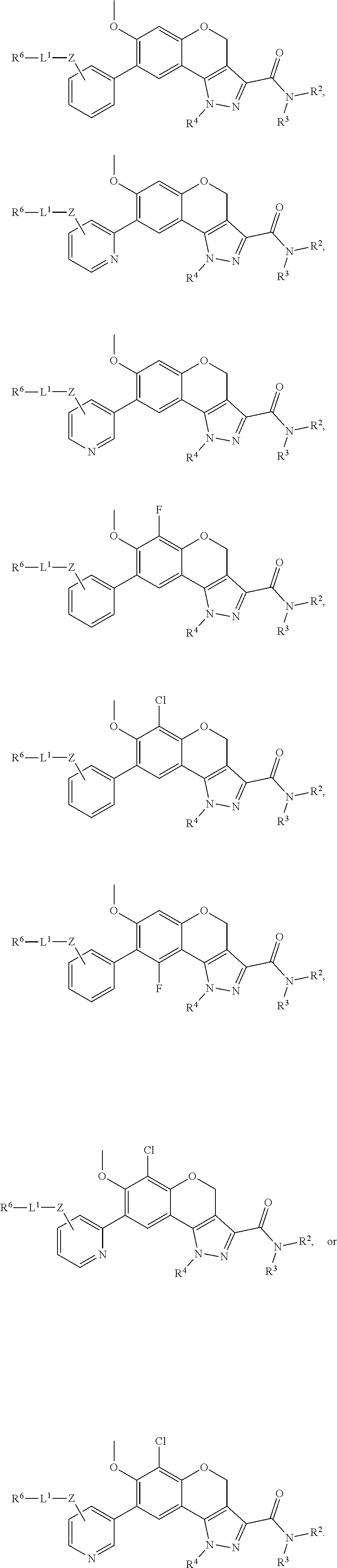

In some embodiments, the compound of Formula (I) has one of the following structures, or a pharmaceutically acceptable salt thereof:

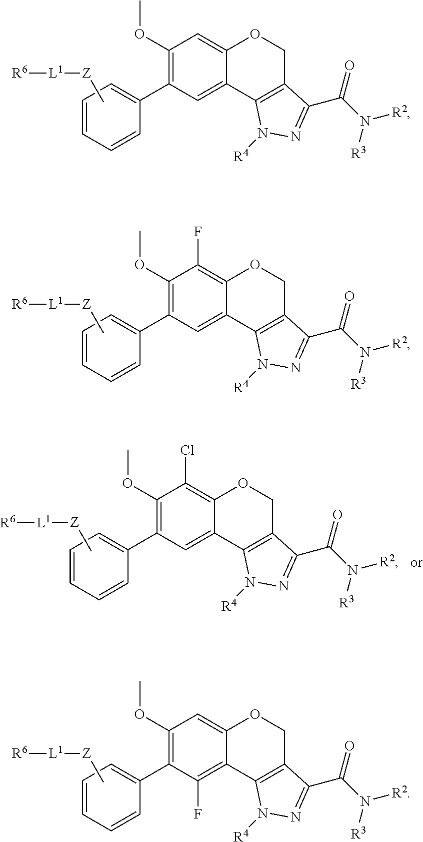

In some embodiments, the compound of Formula (I) has one of the following structures, or a pharmaceutically acceptable salt thereof:

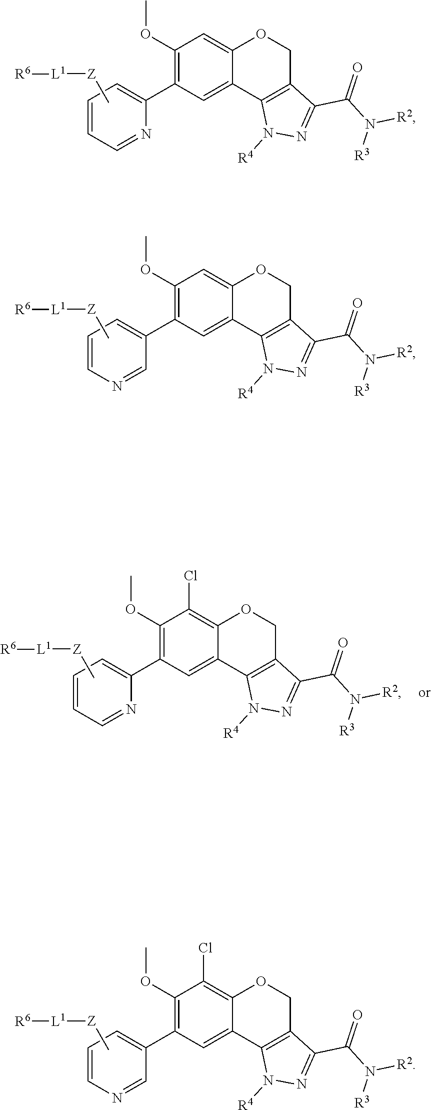

In some embodiments, R² and R³ are each independently substituted or unsubstituted alkyl. In some embodiments, R² is —CH₃ and R³ is t-butyl.

In some embodiments, R² and R³ with the nitrogen to which they are connected form a substituted or unsubstituted 5 to 6-membered heterocycloalkyl. In the aforementioned embodiment, the 5 to 6-membered heterocycloalkyl is substituted with one or two substituents independently selected from the group consisting of H, halogen, substituted or unsubstituted C₁-C₄ alkyl, substituted or unsubstituted C₁-C₄ fluoroalkyl, substituted or unsubstituted 2 to 6 membered heteroalkyl, —C(═O)R^{2c}, —CN, —NH₂, or —OH; wherein R^{2c} is substituted or unsubstituted C₁-C₄ alkyl or substituted or unsubstituted C₃-C₆ cycloalkyl. In some embodiments, R² and R³ with the nitrogen to which they are connected form a substituted or unsubstituted 7 membered heterocycloalkyl. In the aforementioned embodiment, the 7-membered heterocycloalkyl is substituted with one or two substituents independently selected from the group consisting of H, halogen, substituted or unsubstituted C₁-C₄ alkyl, substituted or unsubstituted C₁-C₄ fluoroalkyl, substituted or unsubstituted 2 to 6 membered heteroalkyl, —C(═O) R^{2c}, —CN, —NH₂, or —OH; wherein R^{2c} is substituted or unsubstituted C₁-C₄ alkyl or substituted or unsubstituted C₃-C₆ cycloalkyl. In some embodiments, R² and R³ with the nitrogen to which they are connected form:

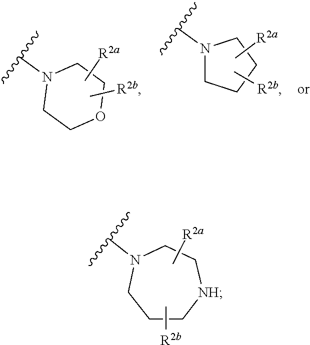

wherein R²a and R^{2b} are each independently H, halogen, substituted or unsubstituted C₁-C₄ alkyl, substituted or unsubstituted C₁-C₄ fluoroalkyl, substituted or unsubstituted 2 to 6 membered heteroalkyl, —C(═O)R^{2c}, —CN, —NH₂, or —OH; wherein R^{2c} is substituted or unsubstituted C₁-C₄ alkyl or substituted or unsubstituted C₃-C₆ cycloalkyl. In some embodiments, R² and R³ with the nitrogen to which they are connected form:

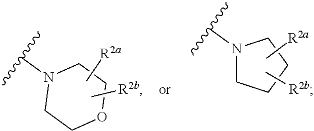

wherein R^{2a} and R^{2b} are each independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —NH₂, or —OH. In some embodiments, R² and R³ with the nitrogen to which they are connected form:

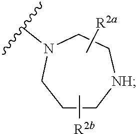

wherein R^{2a} and R^{2b} are each independently H, halogen, substituted or unsubstituted C₁-C₄ alkyl, substituted or unsubstituted C₁-C₄ fluoroalkyl, substituted or unsubstituted 2 to 6 membered heteroalkyl, —C(═O)R^{2c}, —CN, —NH₂, or —OH; wherein R^{2c} is substituted or unsubstituted C₁-C₄ alkyl or substituted or unsubstituted C₃-C₆ cycloalkyl.

In some embodiments, R^{2a} and R²⁰ are each —CH₃. In some embodiments, R^{2a} is H and R^{2b} is —C(—O) R^{2c}; wherein R^{2c} is CH₃, CH₂CH₃, cyclopropyl, cyclobutyl, cyclopentyl or cyclohexyl. In some embodiments, R^{2b} is

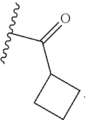 [SMILES: *C(=O)C1CCC1]

In some embodiments, R^{2c} is unsubstituted C₁-C₄ alkyl. In some embodiments, R^{2c} is unsubstituted C₃-C₆ cycloalkyl. In some embodiments, R^{2c} is —CH₃, —CH₂CH₃, cyclopropyl, cyclobutyl, cyclopentyl or cyclohexyl. In some embodiments, R^{2c} is —CH₃. In some embodiments, R^{2c} is —CH₂CH₃. In some embodiments, R^{2c} is cyclopropyl. In some embodiments, R^{2c} is cyclobutyl. In some embodiments, R^{2c} is cyclopentyl. In some embodiments, R^{2c} is cyclohexyl.

In some embodiments, R² and R³ with the nitrogen to which they are connected form:

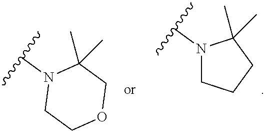

In some embodiments, R² and R³ with the nitrogen to which they are connected form:

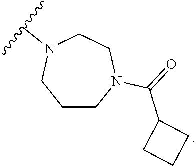 [SMILES: *N1CCCN(C(=O)C2CCC2)CC1]

In some embodiments, R⁴ is a substituted or unsubstituted phenyl or a substituted or unsubstituted 5 to 6-membered heteroaryl.

In some embodiments, R⁴ is a substituted or unsubstituted pyridinyl, substituted or unsubstituted imidazolyl, substituted or unsubstituted pyrimidinyl, substituted or unsubstituted pyrazolyl, substituted or unsubstituted triazolyl, substituted or unsubstituted pyrazinyl, substituted or unsubstituted tetrazolyl, substituted or unsubstituted furyl, substituted or unsubstituted thienyl, substituted or unsubstituted isoxazolyl, substituted or unsubstituted thiazolyl, substituted or unsubstituted oxazolyl, substituted or unsubstituted isothiazolyl, substituted or unsubstituted pyrrolyl, substituted or unsubstituted pyridazinyl, substituted or unsubstituted triazinyl, substituted or unsubstituted oxadiazolyl, substituted or unsubstituted thiadiazolyl, or substituted or unsubstituted furazanyl.

In some embodiments, R⁴ is

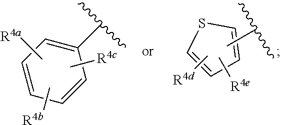

wherein R^{4a}, R^{4b}, R^{4c}, R^{4d} and R^{4e} are each independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —NH₂, or —OH. In some embodiments, R⁴ is

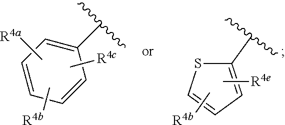

wherein R^{4a}, R^{4b}, R^{4c}, R^{4d} and R^{4e} are each independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —NH₂, or —OH.

In some embodiments, R^{4a} and R^{4b} are each independently F, Cl, Br, I or —CH₃ and R^{4a} is H. In some embodiments, R^{4a} and R^{4b} are each Cl and R⁴ is H.

In some embodiments, R^{4d} and R^{4e} are H.

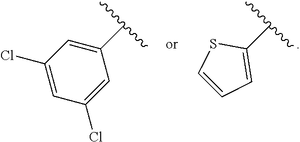 [SMILES: *c1cc(Cl)cc(Cl)c1 ; *c1cccs1]

In some embodiments, R⁴ is In some embodiments, R⁴ is

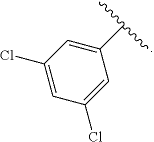 [SMILES: *c1cc(Cl)cc(Cl)c1]

In some embodiments, R⁴ is

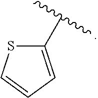 [SMILES: *c1cccs1]

In some embodiments, Z is C₁-C₆ alkylene. In some embodiments, Z is —CH₂—. In some embodiments, Z is —CH₂CH₂—.

In some embodiments, Z is —C(═O)NH—, —NHC(═O)—, —O—, or —NHC(═O)NH—. In some embodiments, Z is —NHC(═O)— or —NHC(═O)NH—. In some embodiments, Z is C₁-C₆ alkylene. In some embodiments, Z is —C(═O)NR¹⁰—. In some embodiments, Z is —C(═O)NH—. In some embodiments, Z is —NR¹⁰C. (═O)—. In some embodiments, Z is —NHC(═O)—. In some embodiments, Z is —NR¹⁰—. In some embodiments, Z is —NH—. In some embodiments, Z is —O—. In some embodiments, Z is —S—. In some embodiments, Z is —S(═O)—. In some embodiments, Z is —SO₂—. In some embodiments, Z is —NR¹⁰C. (═O)NR¹⁰—. In some embodiments, Z is —NHC(═O)NH—. In some embodiments, Z is —CR¹⁰═NO—. In some embodiments, Z is —CH═NO—.

In some embodiments, R⁸ is H. In some embodiments, R⁸ is —N(R¹¹) 2 . . . . In some embodiments, R⁸ is —NH₂. In some embodiments, R⁸ is F. In some embodiments, R⁸ is C₁. In some embodiments, R⁸ is Br. In some embodiments, R⁸ is I. In some embodiments, R⁸ is —OR¹¹ In some embodiments, R⁸ is —OH.

In some embodiments, R⁶ is a chelating moiety selected from the group consisting of: cyclen, DO2A, DO3A, HP-DO3A, DO3A-Nprop, DO3AP, DO3 APPrA, DO3 APABn DO3AM″Bu BT-DO3A, DOTA, DOTAGA, DOTA (GA)₂, DOTAM, DOTA-4AMP, DOTMA, DOTP, CB-DO2A, DOTPA, DOTMP, DOTAMAP, TRITA, LPY, cyclam, TETA, CB-Cyclam, CB-TE2A, TE2A, NOTA, NODAGA, NODA-MPAA, TACN, TACN-TM, NOTP, Sarcophagine (Sar), DiAmSar, SarAr, AmBaSar, cis —DO2A2P, trans-DO2A2P, DOTEP, p-NO2-Bn-DOTA, BAT, DO3TMP-Monoamide, CHX-A″-DTPA, c-DEPA, PCTA, p-NO2-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, Hapy4pa, CP256, THP, YM103, t-Bu-calix [4]arene-tetracarboxylic acid, CHX-A″-DTPA, Hophospha, p-NH₂-Bn-CHXA″-DTPA, DEDPA, H₄octox, H₄octapa, H4CHXoctapa, HYNIC, macropa, crown, macropid, HOPO, Bis(2-mercaptoacetamide), Bis(aminothiolate), or SBTG2DAP.

In some embodiments, R⁶ is a chelating moiety selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))-tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2″-(1,10-dioxa-4, 7, 13, 16-tetraazacyclooctadecane-4, 7, 13, 16-tetrayl)tetraacetic acid (crown); 6,6′-((ethane-1,2-diylbis((carboxymethyl)azanediyl))bis(methylene))dipicolinic acid (H₄octapa); H₄octapa-benzyl; 3,6,9,12-tetrakis(carboxymethyl)-3,6,9,12-tetraazatetradecanedioic acid (TTHA); or a radionuclide complex thereof.

In some embodiments, R⁶ is a chelating moiety selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; 6,6′-(((pyridine-2,6-diylbis(methylene))bis((carboxymethyl)azanediyl))bis(methylene))dipicolinic acid (H₄pypa); 6,6′,6″,6″-(((pyridine-2,6-diylbis(methylene))bis(azanetriyl))tetrakis(methylene))-tetrapicolinic acid (H₄py4pa); 2,2′,2″-(1,4,7-triazacyclononane-1,4,7-triyl)triacetic acid (NOTA); 6,6′-((1,4, 10, 13-tetraoxa-7, 16-diazacyclooctadecane-7,16-diyl)bis(methylene))dipicolinic acid (macropa); 2,2′,2″,2″-(1, 10-dioxa-4,7,13, 16-tetraazacyclooctadecane-4,7, 13, 16-tetrayl)tetraacetic acid (crown); 6,6′-((ethane-1,2-diylbis((carboxymethyl)azanediyl))bis(methylene))dipicolinic acid (H₄octapa); 3,6,9,12-tetrakis(carboxymethyl)-3,6,9,12-tetraazatetradecanedioic acid (TTHA); or a radionuclide complex thereof. In some embodiments, R⁶ is 1,4,7,10-tetraazacyclododecane-1,4,7,10-tetraacetic acid (DOTA) or a radionuclide complex thereof. In some embodiments, R⁶ is 1,4,7,10-tetraazacyclododecane-1,4,7-triacetic acid (DO3A) or a radionuclide complex thereof. In some embodiments, R⁶ is 1,4,7,10-tetraazacyclododecane-1,7-diacetic acid (DO2A) or a radionuclide complex thereof. In some embodiments, R^{w is α,α′,α″, ″-tetramethyl-}1,4,7,10-tetraazacyclododecane-1,4,7,10-tetraacetic acid (DOTMA) or a radionuclide complex thereof. In some embodiments, R⁶ is 1,4,7,10-tetrakis(carbamoylmethyl)-1,4,7,10-tetraazacyclododecane (DOTAM) or a radionuclide complex thereof. In some embodiments, R⁶ is 1,4,7,10-tetraazacyclododecane-1,4,7,10-tetrapropionic acid (DOTPA) or a radionuclide complex thereof. In some embodiments, R⁶ is 2,2′,2″-(10-(2-amino-2-oxoethyl)-1,4,7,10-tetraazacyclododecane-1,4,7-triyl)triacetic acid or a radionuclide complex thereof. In some embodiments, R⁶ is 6,6′-(((pyridine-2,6-diylbis(methylene))bis((carboxymethyl)azanediyl))bis(methylene))dipicolinic acid (H₄pypa) or a radionuclide complex thereof. In some embodiments, R⁶ is 6,6′,6″,6′-(((pyridine-2,6-diylbis(methylene))bis(azanetriyl))tetrakis(methylene))-tetrapicolinic acid (H₄py4pa) or a radionuclide complex thereof. In some embodiments, R⁶ is 2,2′,2″-(1,4,7-triazacyclononane-1,4,7-triyl)triacetic acid (NOTA). In some embodiments, R⁶ is 6,6′-((1,4,10,13-tetraoxa-7,16-diazacyclooctadecane-7,16-diyl)bis(methylene))dipicolinic acid (macropa). In some embodiments, R⁶ is 2,2′,2″,2′-(1,10-dioxa-4,7, 13, 16-tetraazacyclooctadecane-4,7,13, 16-tetrayl)tetraacetic acid (crown). In some embodiments, R⁶ is 6,6′-((ethane-1,2-diylbis((carboxymethyl)azanediyl))bis(methylene))dipicolinic acid (H₄octapa) or a radionuclide complex thereof. In some embodiments, R⁶ is 3,6,9,12-tetrakis(carboxymethyl)-3,6,9,12-tetraazatetradecanedioic acid (TTHA) or a radionuclide complex thereof.

In some embodiments, R⁶ is a chelating moiety selected from the group consisting of: 1,4,7,10-tetraazacyclododecane-1,4,7,10-tetraacetic acid (DOTA) or 1,4,7,10-tetraazacyclododecane-1,4,7-triacetic acid (DO3A); or a radionuclide complex thereof.

In some embodiments, R⁶ is a chelating moiety selected from the group consisting of:

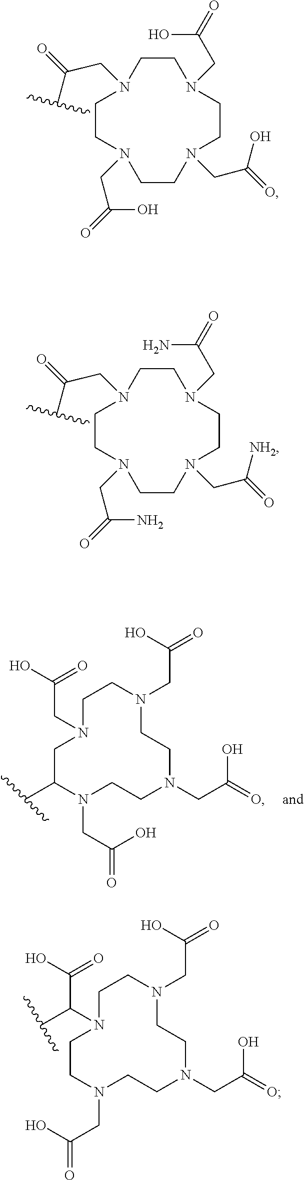 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]

or a radionuclide complex thereof.

In some embodiments, R⁶ is

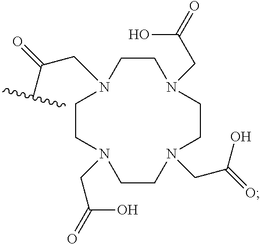 [SMILES: *C(=O)CN1CCN(CC(=O)O)CCN(CC(=O)O)CCN(CC(=O)O)CC1]

or a radionuclide complex thereof.

In some embodiments, R⁶ is

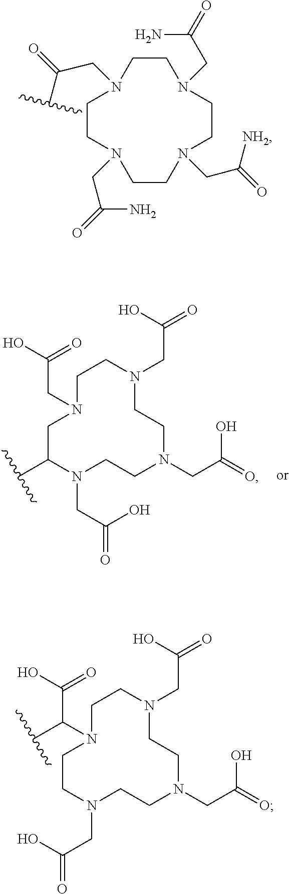 [SMILES: *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; "or" ; *C(C(=O)O)N1CCN(CC(=O)O)CCN(CC(=O)O)CCN(CC(=O)O)CC1]

or a radionuclide complex thereof.

In some embodiments, R⁶ is

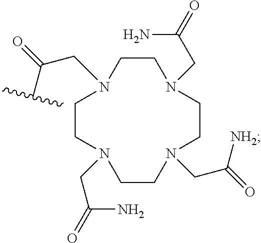 [SMILES: *C(=O)CN1CCN(CC(N)=O)CCN(CC(N)=O)CCN(CC(N)=O)CC1]

or a radionuclide complex thereof.

In some embodiments, R⁶ is

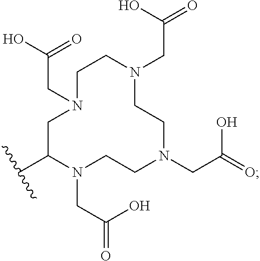 [SMILES: *C1CN(CC(=O)O)CCN(CC(=O)O)CCN(CC(=O)O)CCN1CC(=O)O]

or a radionuclide complex thereof.

In some embodiments, R⁶ is

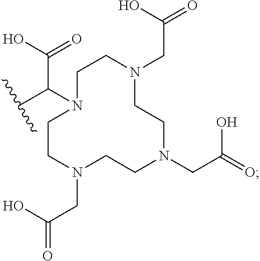 [SMILES: *C(C(=O)O)N1CCN(CC(=O)O)CCN(CC(=O)O)CCN(CC(=O)O)CC1]

or a radionuclide complex thereof.

### Radionuclide Complexes

Radiopharmaceuticals have increasingly become very useful tools for physicians to diagnose, stage, treat, and monitor the progression of several diseases, especially cancer. The primary difference between radiopharmaceuticals and other pharmaceutical drugs is that radiopharmaceuticals contain a radionuclide. The nuclear decay properties of the radionuclide determine whether a radiopharmaceutical will be used clinically as a diagnostic agent or as a therapeutic agent. Diagnostic radiopharmaceuticals require radionuclides that emit either gamma (γ) rays or positrons (β+), which subsequently annihilate with nearby electrons to produce two 511 keV annihilation photons emitted approximately 180° away from each other. Gamma ray-emitting radionuclides (e. g. 99mTc, 111 In, 201Tl, etc.) are useful for single photon emission computed tomography (SPECT), while positron-emitting radionuclides (e. g. 18F, 89Zr, 68Ga, etc.) are useful for positron emission tomography (PET).

In contrast, therapeutic radiopharmaceuticals require radionuclides that emit particulate radiation, such as alpha (α) particles, beta (β−) particles, or Auger electrons. These particles, which strongly interact with target tissues (e. g. cancerous tumor) and lead to extensive localized ionization, can damage chemical bonds in DNA molecules and potentially induce cytotoxicity.

For most nuclear medicine applications, it is desired that a diagnostic radiopharmaceutical is paired with a therapeutic radiopharmaceutical. This concept is commonly known as “theranostics”. As a first step in the theranostic concept, a target molecule labeled with a diagnostic radionuclide is used for quantitative imaging of a tumor imaging biomarker, either by positron emission tomography (PET) or single photon emission computed tomography (SPECT). When it is demonstrated that, with this targeted molecule, a tumoricidal radiation absorbed dose can be delivered to tumor and metastases, as a second step, via administration of the same or a similar target molecule labeled with a therapeutic radionuclide.

In some embodiments, the chemical and pharmacokinetic behaviors of both the diagnostic and therapeutic radiopharmaceuticals match. In some embodiments, the diagnostic and therapeutic radionuclides are a chemically identical radioisotope pair (also known as a “matched pair”). One examples of a matched pair for theranostic radiopharmaceutical applications is the 1231/1311 pair, where 1231-labeled compounds are used for diagnosis, while 1311-labeled compounds are used for therapy. Other theranostic matched pairs include++Sc/47Sc, ⁶⁴Cu/⁶⁷Cu, ⁷² As/⁷⁷As, ⁸⁶Y/⁹⁰Y, and ²⁰³Pb/²¹²Pb, among others. Alternatively, radionuclide pairs from different elements can be utilized for theranostic radiopharmaceutical development when their chemistry is very similar (e. g. ^{99m}Tc/^{186/188}Re) and there is no significant difference in the pharmacokinetic behavior between the diagnostic and therapeutic analogues. Another example is the ⁶⁸Ga/¹⁷⁷Lu pair, where ⁶⁸Ga is used for diagnosis and ¹⁷⁷Lu is used for therapy. For example, gastroenteropancreatic endocrine tumors express high amounts of sst2 receptor that can be targeted with somatostatin receptor scintigraphy for diagnostic purposes with a ⁶⁸Ga sst2 ligand conjugate ([⁶⁸Ga]Ga-DOTA-TATE (NETSPOT™) or [⁶⁸Ga]Ga-DOTA-TOC (DOTA-(D-Phel, Tyr3)-octreotide, SomaKit TOC®)), followed by treatment with a 177Lu sst2 ligand conjugate ([177Lu]Lu-DOTA-TATE) for endoradiotherapy.

Chelating Moieties used to Generate Metal (Radionuclide) Complexes

The compounds described herein comprise at least one R⁶ group, wherein R⁶ is chelating moiety capable of chelating a radionuclide (Z′), or radionuclide complex thereof. In some embodiments, any suitable group or atom(s) of the chelator are used to connect, via an optional linker, to the FSHR targeting ligand.

In some embodiments, the chelator is capable of binding a radioactive atom. In some embodiments, the binding is direct, e.g., the chelator makes hydrogen bonds or electrostatic interactions with a radioactive atom. In some embodiments, the binding is indirect, e.g., the chelator binds to a molecule that comprises a radioactive atom. In some embodiments, the chelator is or comprises a macrocycle.

In some embodiments, the chelator comprises one or more amine groups. In some embodiments, the metal chelator comprises two or more amine groups. In some embodiments, the chelator comprises three or more amine groups. In some embodiments, the chelator comprises four or more amine groups. In some embodiments, the chelator includes 4 or more N atoms, 4 or more carboxylic acid groups, or a combination thereof. In some embodiments, the chelator does not comprise S. In some embodiments, the chelator comprises a ring. In some embodiments, the ring comprises an O and/or a N atom. In some embodiments, the chelator is a ring that includes 3 or more N atoms, 3 or more carboxylic acid groups, or a combination thereof. In some embodiments, the chelator is polydentate ligand, bidentate ligand, or monodentate ligand. Polydentate ligands range in the number of atoms used to bond to a metal atom or ion. EDTA, a hexadentate ligand, is an example of a polydentate ligand that has six donor atoms with electron pairs that can be used to bond to a central metal atom or ion. Bidentate ligands have two donor atoms which allow them to bind to a central metal atom or ion at two points. Ethylenediamine (en) and the oxalate ion (ox) are examples of bidentate ligands.

In some embodiments, a chelator described herein comprises a cyclic chelating agent or an acyclic chelating agent. In some embodiments, a chelator described herein comprises a cyclic chelating agent. In some embodiments, a chelator described herein comprises an acyclic chelating agent.

In some embodiments, a chelator described herein comprises cyclen, DO2A, DO3A, HP-DO3A, DO3A-Nprop, DO3AP, DO3 APPrA, DO3APABIL DO3AM″Bu, BT-DO3A, DOTA, DOTAGA, DOTA (GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE₂A, TE2A, NOTA, NODAGA, NODA-MPAA, TACN, TACN-TM, NOTP, Sarcophagine (Sar), DiAmSar, SarAr, AmBaSar, cis —DO2A2P, trans-DO2A2P, DOTEP, p-NO₂-Bn-DOTA, BAT, DO3TMP-Monoamide, CHX-A″-DTPA, c-DEPA, PCTA, p-NO₂-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄ypa, H₄py4pa, CP256, THP, YM103, t-Bu-calix [4]arene-tetracarboxylic acid, CHX-A″-DTPA, Hophospha, p-NH₂-Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), or SBTG₂DAP.

In some embodiments, a chelator described herein comprises DOTA, DOTAGA, DOTA(GA)₂, NOTA, NODAGA, TRITA, TETA, DOTA-MA, HP-DO3A, DOTMA, DOTA-pNB, DOTP, DOTMP, DOTEP, DOTMPE, F-DOTPME, DOTPP, DOTBzP, DOTA-monoamide, BAT, DO3TMP-Monoamide, and CHX-A″-DTPA.

In some embodiments, a chelator described herein comprises DTA, CyEDTA, EDTMP, DTPMP, DTPA, CyDTPA, Cy2DTPA, DTPA-MA, DTPA-BA, and BOPA.

In some embodiments, a chelator described herein comprises DOTA, DOTAGA, DOTA (GA)₂, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP.

In some embodiments, a chelator described herein comprises HP-DO3A, BT-DO3A, DO3A-Nprop, DO3AP, DO2A2P, DOA3P, DOTP, DOTPMB, DOTAMAE, DOTAMAP, DO3AMBu, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA″, THP, HEHA, HBED, or HBED-CC TFP.

In some embodiments, a chelator described herein comprises DOTA, NOTA, NODAGA, DOTAGA, HBED, HBED-CC TFP, H2DEPDPA, DFO—B, Deferiprone, CP256, YM103, TETA, CB-TE2A, TE2A, Sar, DiAmSar, TRAPH, TRAP-Pr, TRAP-OH, TRAP-Ph, NOPO, DEADPA, PCTA, EDTA, PEPA, HEHA, DTPA, EDTMP, AAZTA, DO3AP, DO3 AP^{PrA}, DO3 AP^{ABn}, or DOTAM.

In some embodiments, the chelator is or comprises DOTA, HBED-CC, DOTAGA, DOTA(GA)₂, NOTA, and DOTAM. In some embodiments, the chelator is or comprises NODAGA, NOTA, DOTAGA, DOTA (GA)₂, TRAP, NOPO, NCTA, DFO, DTPA, and HYNIC.

In some embodiments, the chelator comprises a macrocycle, e.g., a macrocycle comprising an O and/or a N atom, DOTA, HBED-CC, DOTAGA, DOTA (GA)₂, NOTA, DOTAM, one or more amines, one or more ethers, one or more carboxylic acids, EDTA, DTPA, TETA, DO3A, PCTA, or desferrioxamine.

In some embodiments, a metal chelator described herein comprises one of the following structures:

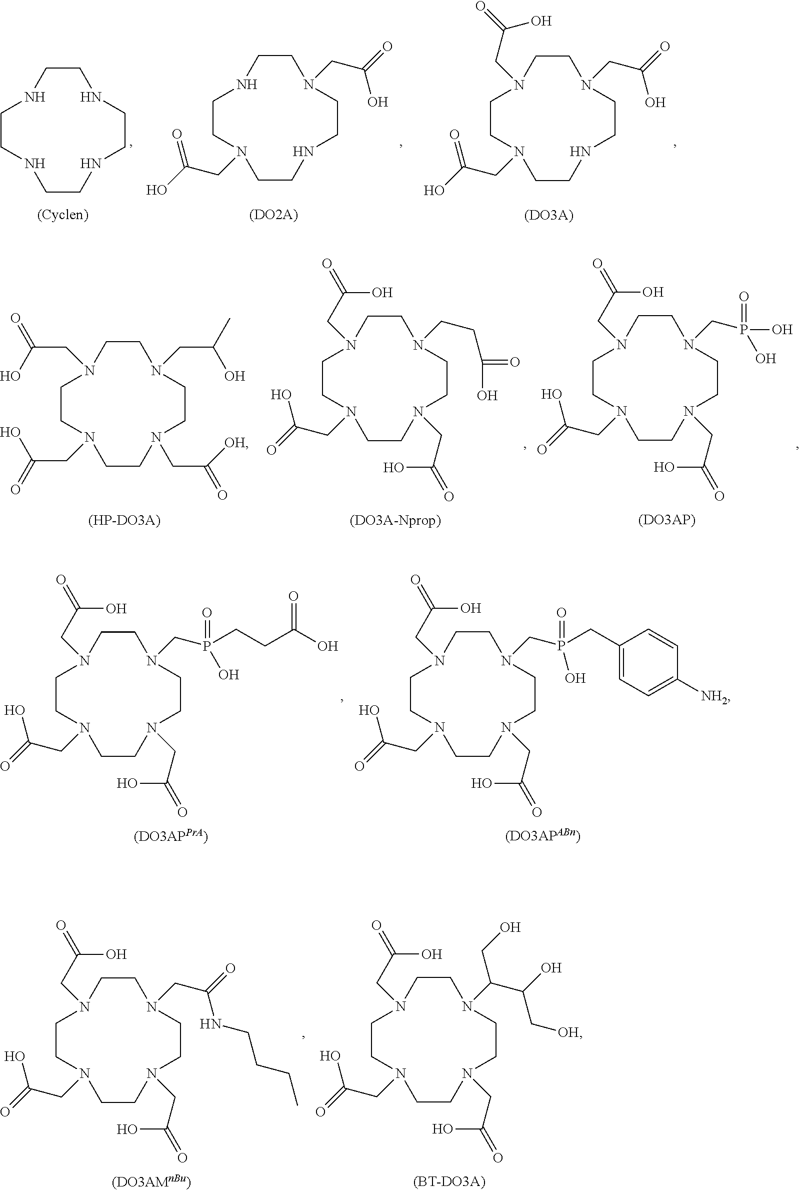

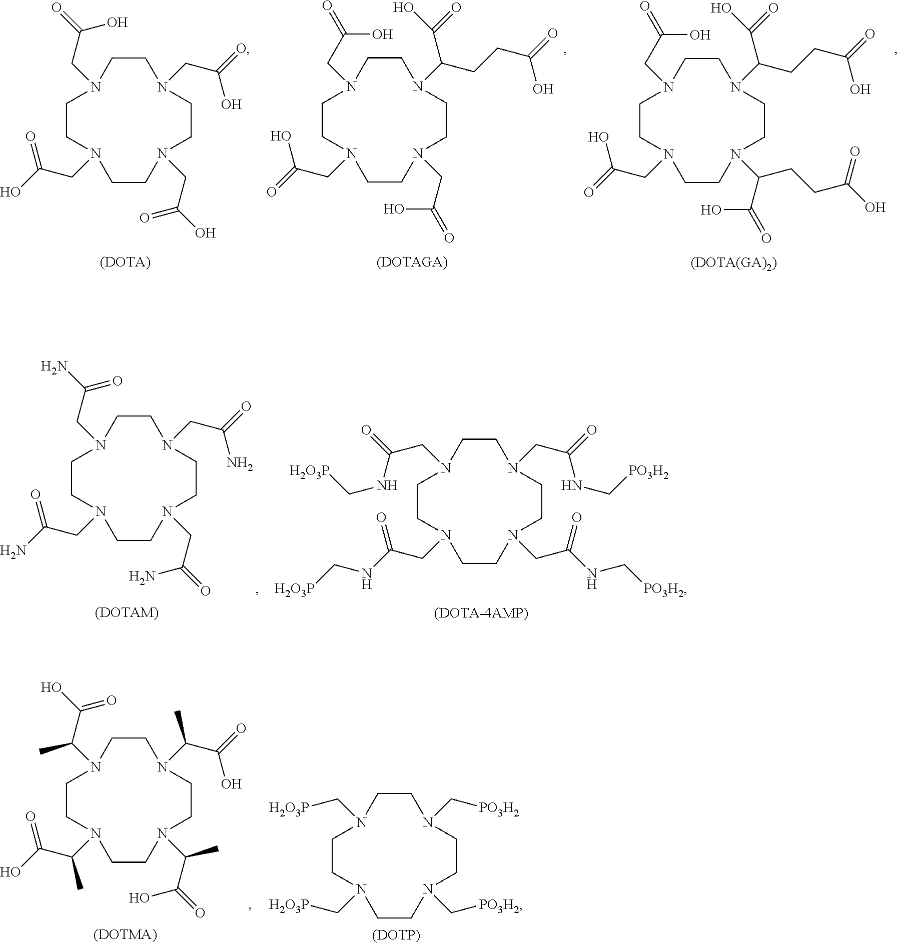

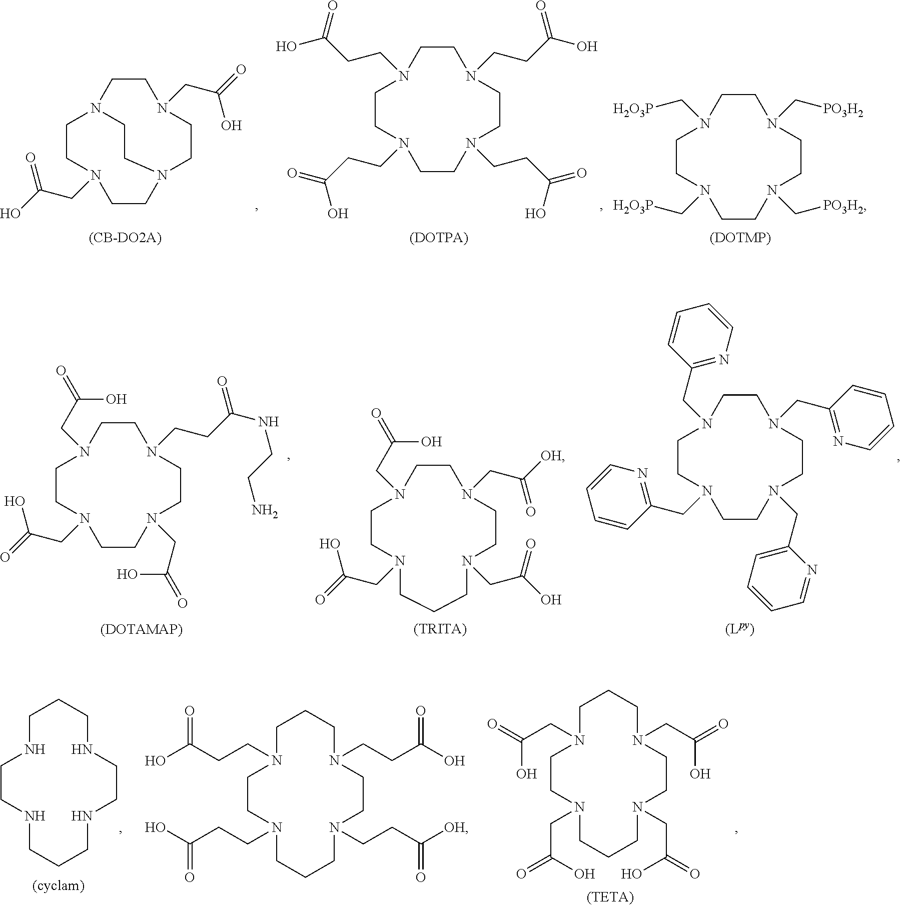

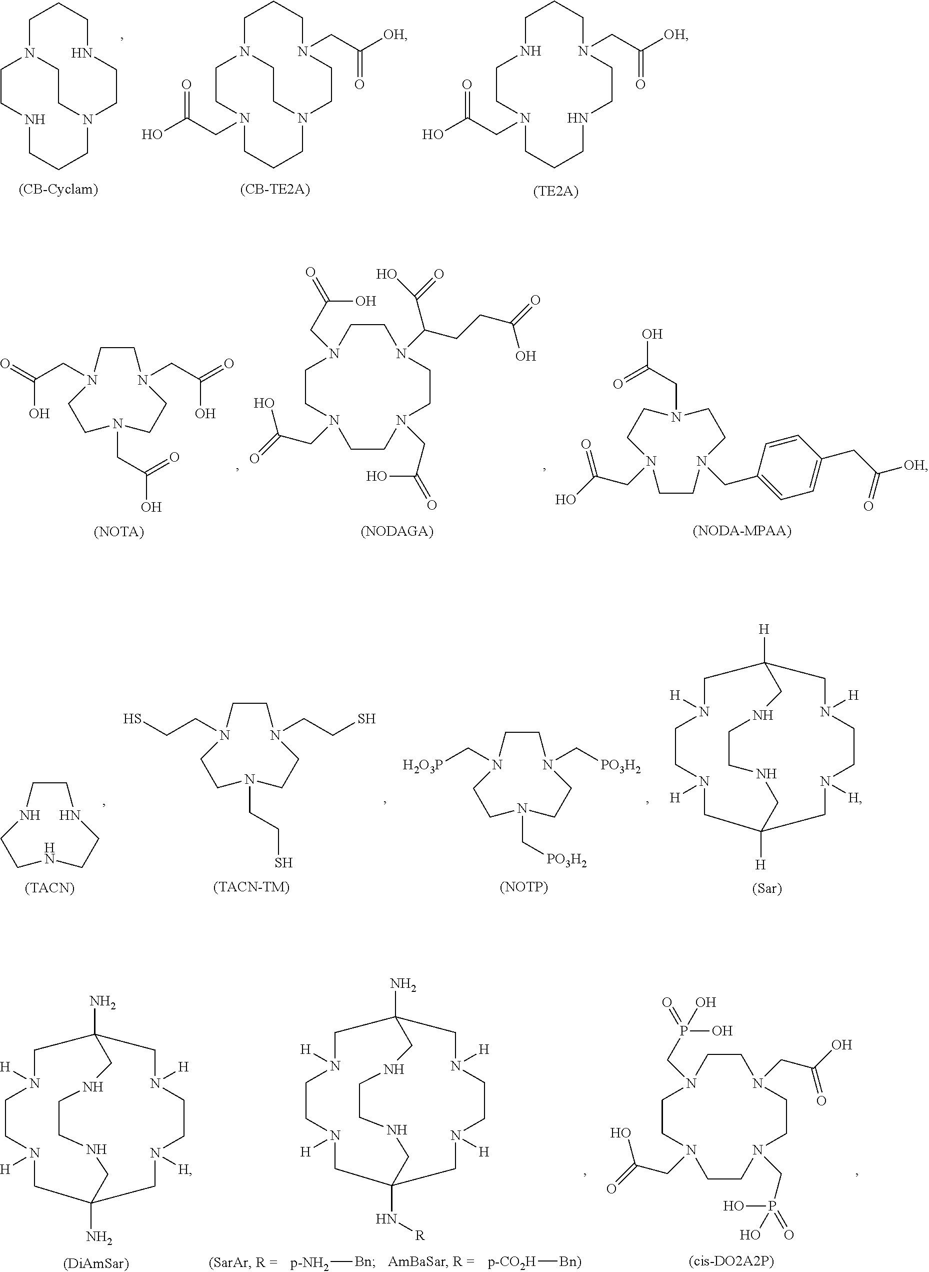

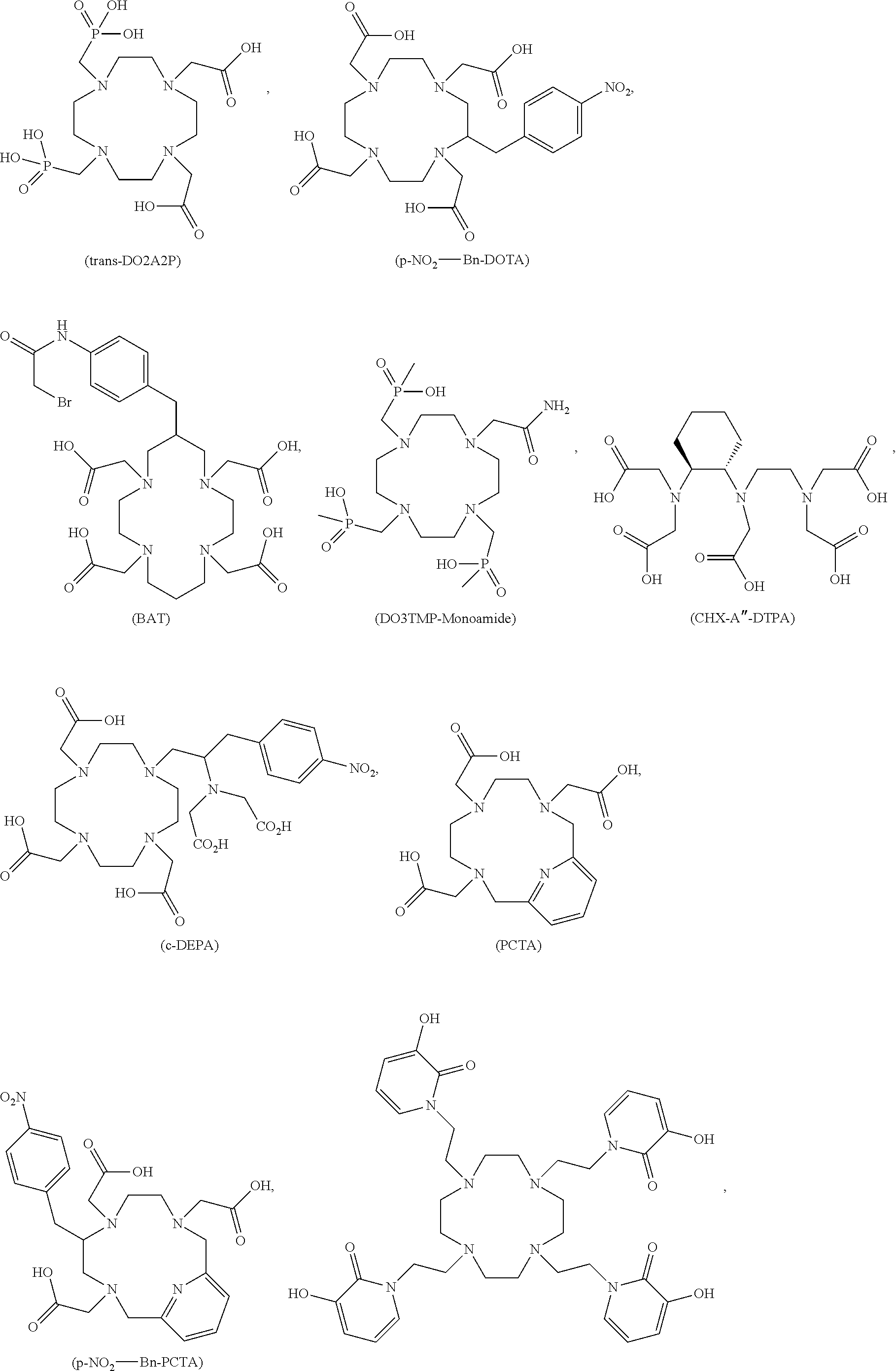

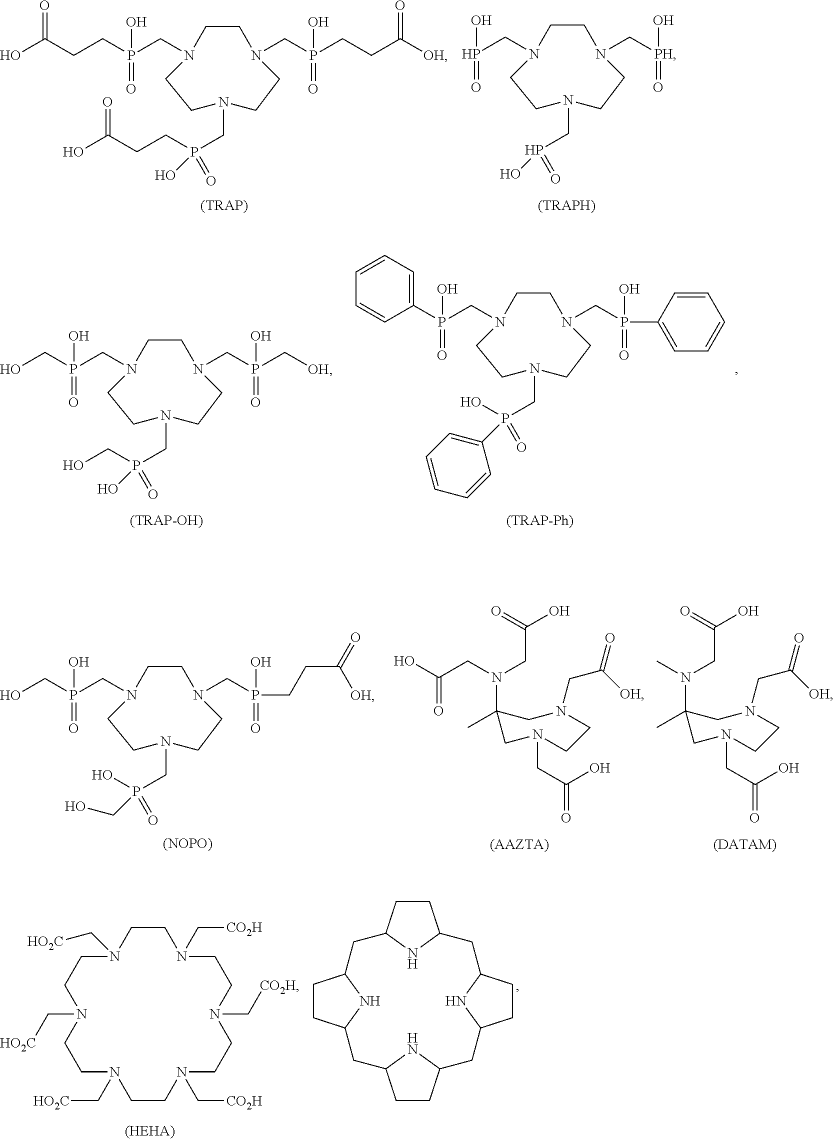

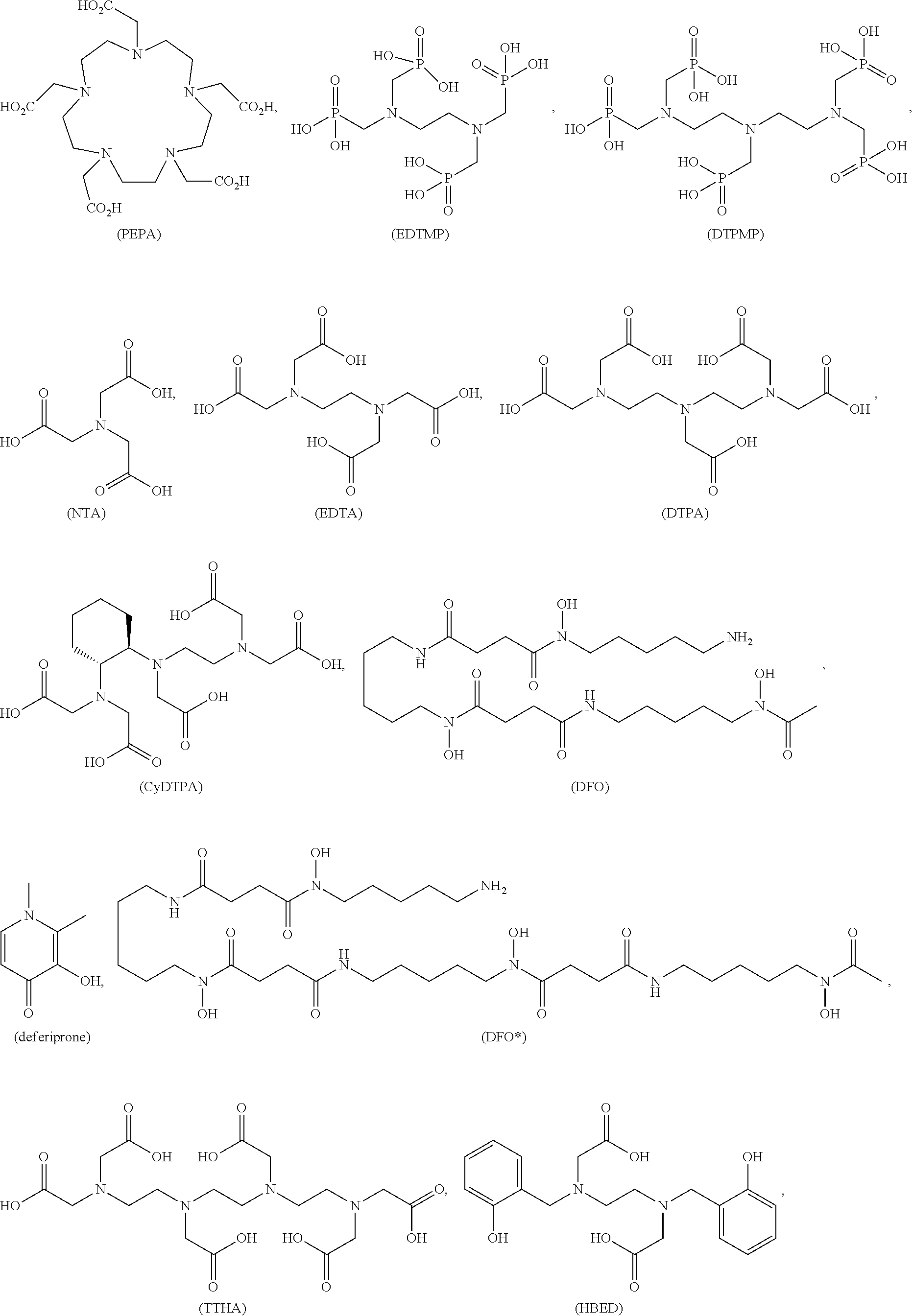

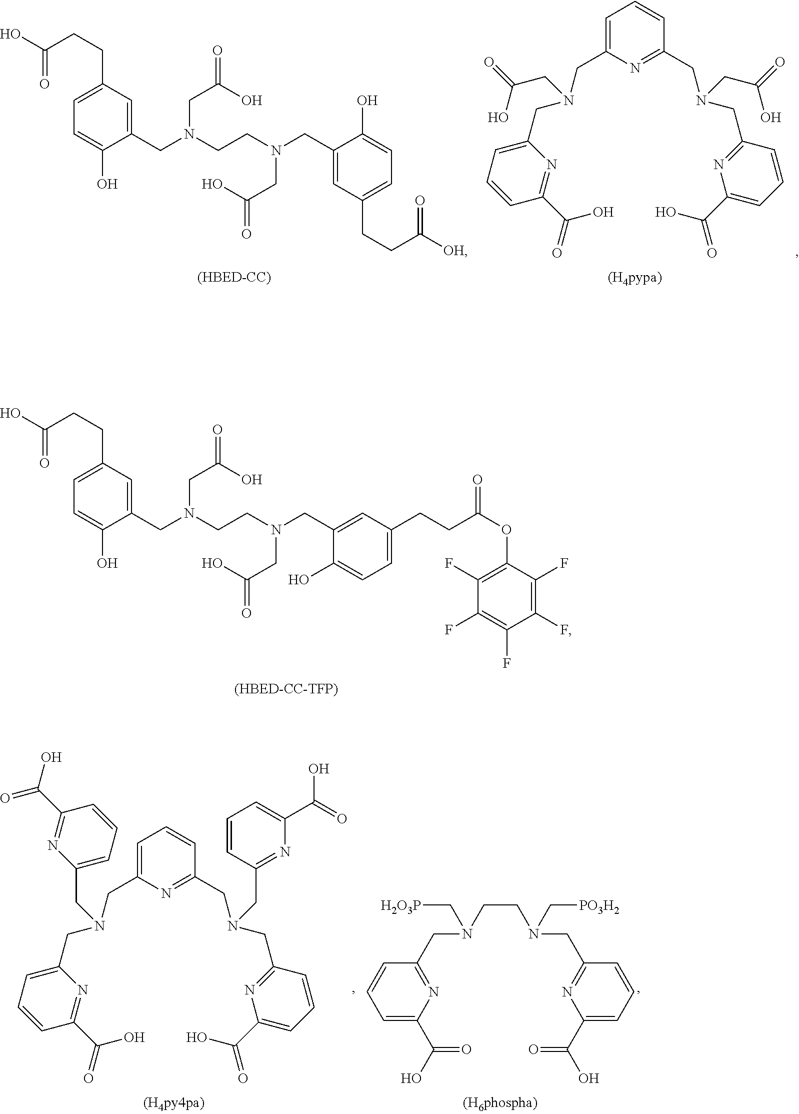

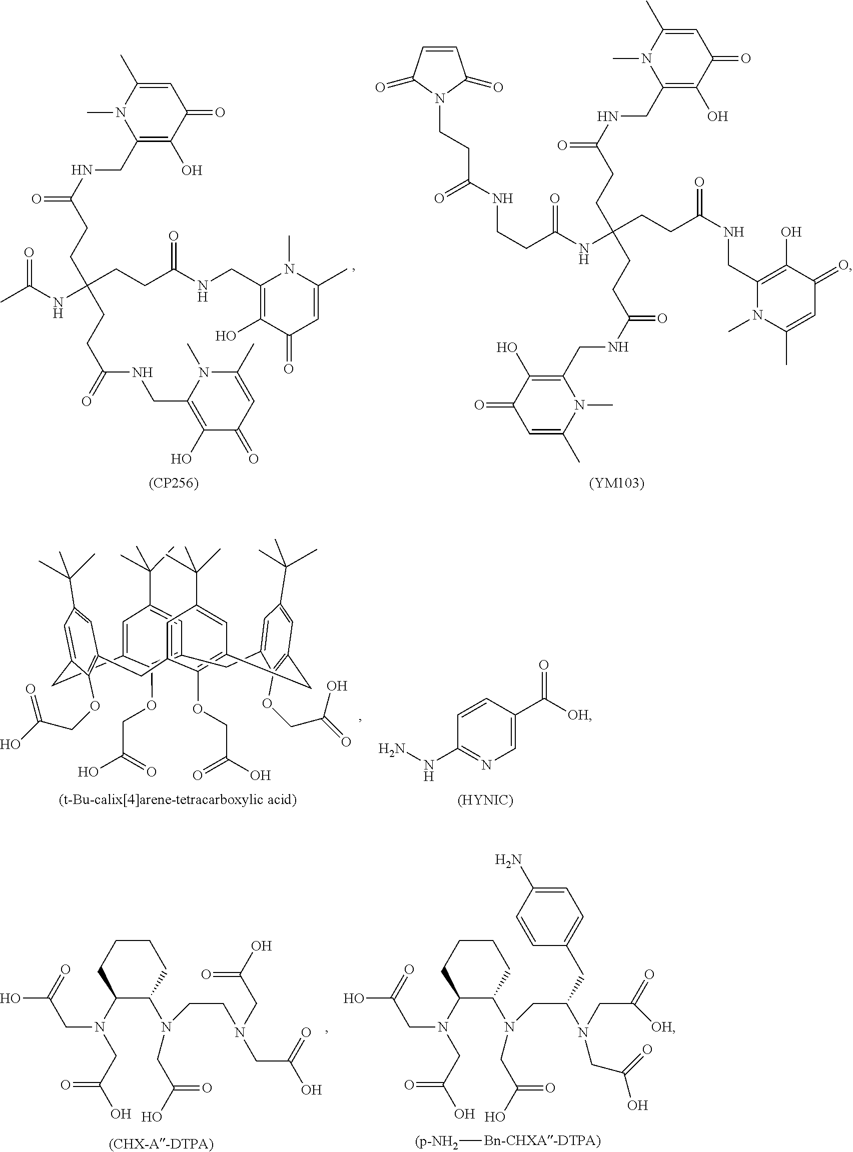

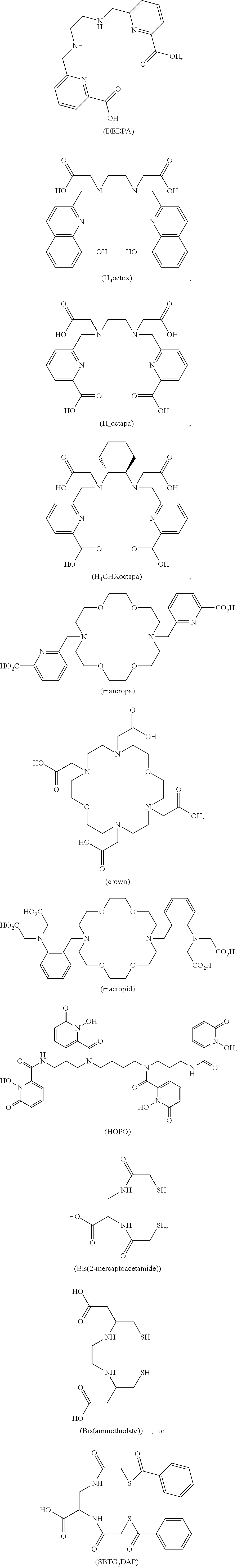

In some embodiments, the chelating moiety R⁶ comprises a radionuclide and DOTA. In some embodiments, the chelating moiety R⁶ comprises a radionuclide and a DOTA derivative. In some embodiments, the chelating moiety comprises two independent chelators, and at least one or both are DOTA.

In some embodiments, the chelating moiety comprises a radionuclide and a chelator configured to bind the radionuclide (Z′), wherein the chelator comprises DOTA, DOTP, DOTMA, DOTAM, DTPA, NOTA, NTA, NODAGA, EDTA, DO3A, DO2A, NOC, TETA, CB-TE2A, DiAmSar, CB-Cyclam, DOTA-4AMP, H₄pypa, Hoctox, H₄octapa, p-NO₂-Bn-neunpa, or NOTP.

In some embodiments, the metal chelator described herein comprises macropa or crown. In some embodiments, the metal chelator described herein comprises macropa. In some embodiments, the metal chelator described herein comprises crown. In some embodiments, the metal chelator described herein comprises

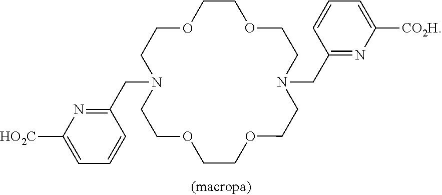 [SMILES: O=C(O)c1cccc(CN2CCOCCOCCN(Cc3cccc(C(=O)O)n3)CCOCCOCC2)n1]

In some embodiments, the metal chelator described herein comprises

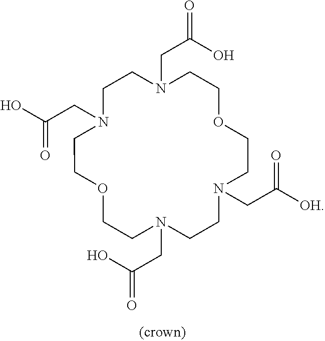 [SMILES: O=C(O)CN1CCOCCN(CC(=O)O)CCN(CC(=O)O)CCOCCN(CC(=O)O)CC1]

In some embodiments, R⁶ is a chelating moiety selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))-tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10, 13-tetraoxa-7,16-diazacyclooctadecane-7,16-diyl)bis(methylene))dipicolinic acid (macropa); 2,2′,2″,2″-(1,10-dioxa-4,7, 13, 16-tetraazacyclooctadecane-4,7,13, 16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof. In some embodiments, R⁶ is a chelating moiety selected from the group consisting of: DOTA and DO3A; or a radionuclide complex thereof.

In some embodiments, R⁶ is a chelating moiety selected from the group consisting of:

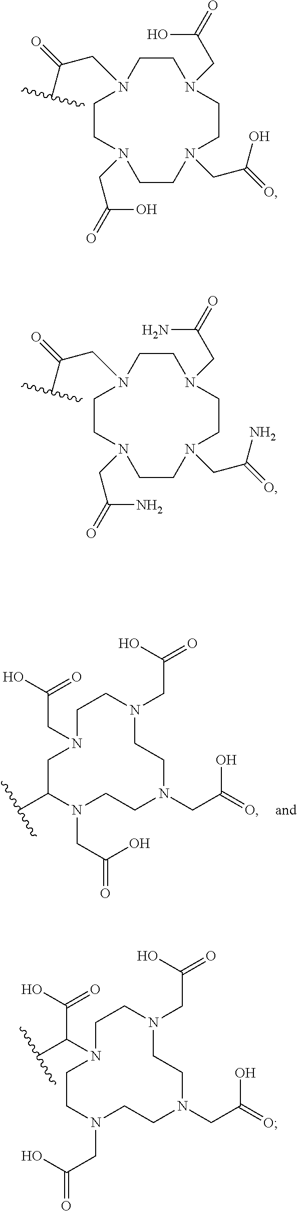 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof. In some embodiments, R⁶ is:

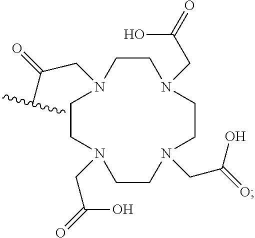 [SMILES: *C(=O)CN1CCN(CC(=O)O)CCN(CC(=O)O)CCN(CC(=O)O)CC1]

or a radionuclide complex thereof. In some embodiments, R⁶ is:

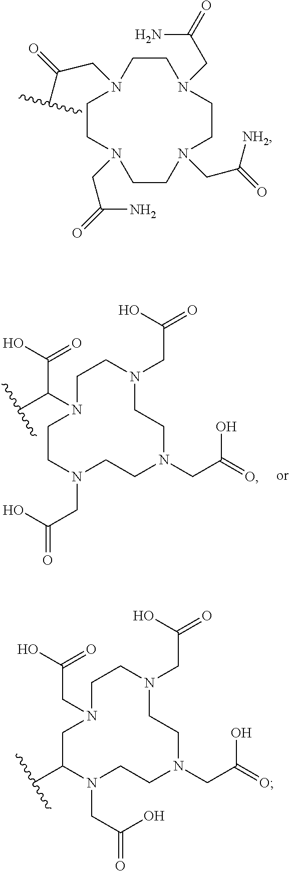 [SMILES: *C(=O)CN1CCN(CC(N)=O)CCN(CC(N)=O)CCN(CC(N)=O)CC1 ; *C(C(=O)O)N1CCN(CC(=O)O)CCN(CC(=O)O)CCN(CC(=O)O)CC1 ; "or" ; *C1CN(CC(=O)O)CCN(CC(=O)O)CCN(CC(=O)O)CCN1CC(=O)O]

or a radionuclide complex thereof.

In some embodiments, R⁶ is:

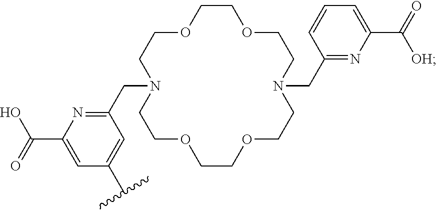 [SMILES: *c1cc(CN2CCOCCOCCN(Cc3cccc(C(=O)O)n3)CCOCCOCC2)nc(C(=O)O)c1]

or a radionuclide complex thereof. In some embodiments, R⁶ is:

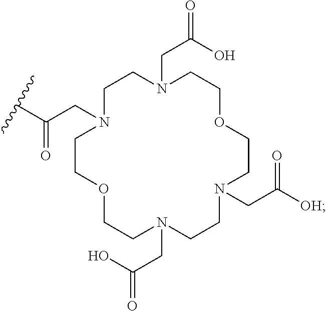 [SMILES: *C(=O)CN1CCOCCN(CC(=O)O)CCN(CC(=O)O)CCOCCN(CC(=O)O)CC1]

or a radionuclide complex thereof.

In some embodiments, R⁶ is:

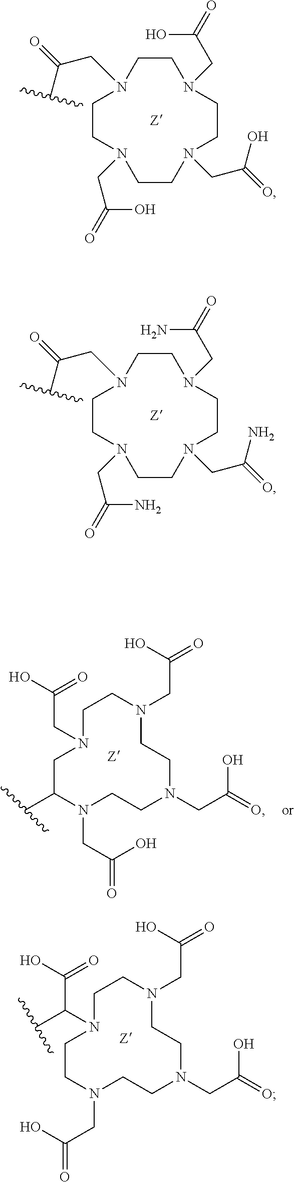

wherein Z′ is a diagnostic or therapeutic radionuclide.

In some embodiments, R⁶ is:

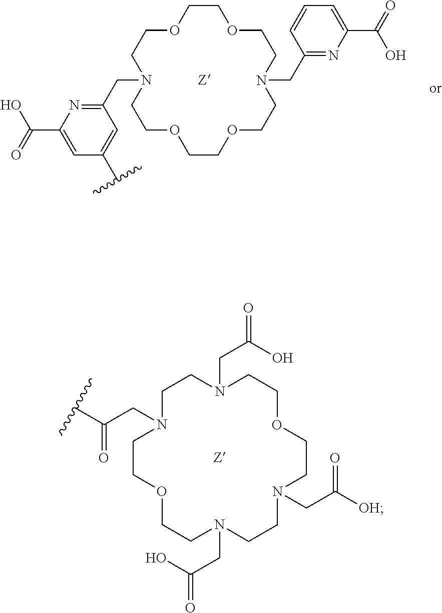

wherein Z′ is a diagnostic or therapeutic radionuclide.

In some embodiments, Z′ is an Auger electron-emitting radionuclide, α-emitting radionuclide, β-emitting radionuclide, or γ-emitting radionuclide. In some embodiments, Z′ is an Auger electron-emitting radionuclide that is 111-indium (¹¹¹ In), 67-gallium (⁶⁷Ga), 68gallium (⁶⁸Ga), 99m-technetium (^{99m}Tc), or 195m-platinum (^{195m}Pt). In some embodiments, Z′ is an α-emitting radionuclide that is 225-actinium (²²⁵ Ac), 213-bismuth (²¹³Bi), 223-Radium (²²³Ra), or 212-lead (²¹²Pb). In some embodiments, Z′ is a β-emitting radionuclide that is 90-yttrium (9Y), 177-lutetium (¹⁷⁷Lu), iodine-131 (¹³¹I), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn). In some embodiments, Z′ is a γ-emitting radionuclide that is 60-cobalt (⁶⁰Co), 103-palldium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, R⁶ comprises a radionuclide (Z′) and a chelator configured to bind the radionuclide (Z′), wherein the radionuclide is suitable for positron emission tomography (PET) analysis, single-photon emission computerized tomography (SPECT), or magnetic resonance imaging (MRI). In some embodiments, the radionuclide is copper-64 (⁶⁴Cu), gallium-68 (⁶⁸Ga), 111-indium (¹¹¹ In), or technetium-99m (^{99m}Tc).

### Metals (Radionuclides)

In some embodiments, Z′ is an Auger electron-emitting radionuclide. In some embodiments, Z′ is an α-emitting radionuclide. In some embodiments, Z′ is a β-emitting radionuclide. In some embodiments, Z′ is a γ-emitting radionuclide. In some embodiments, the type of radionuclide used in a non-peptide targeted therapeutic compound can be tailored to the specific type of cancer, the type of targeting moiety (e.g., non-peptide ligand), etc. Radionuclides that undergo α-decay emit α-particles (helium ions with a +2 charge) from their nuclei. As a result of α-decay the daughter nuclide has 2 protons less and 2 neutrons less than the parent nuclide. This means that in α-decay, the proton number is reduced by 2 while the nucleon number is reduced by 4. Radionuclides that undergo β-decay emit β-particles (electrons) from their nuclei. During β-decay, one of the neutrons changes into a proton and an electron. The proton remains in the nucleus while the electron is emitted as a β-particle. This means that in β-decay, the nucleus loses a neutron but gains a proton. In γ-decay, a nucleus in an excited state (higher energy state) emits a γ-ray photon to change to a lower energy state. There is no change in the proton number and nucleon number during the γ-decay. The emission of γ-rays often accompanies the emission of α-particles and β-particles. Auger electrons (AEs) are very low energy electrons that are emitted by radionuclides that decay by electron capture (EC) (e.g. 111 In, 67Ga, 99mTc, 195mpt, 1251 and 1231). This energy is deposited over nanometer-micrometer distances, resulting in high linear energy transfer that is potent for causing lethal damage in cancer cells. Thus, AE-emitting radiotherapeutic agents have great potential for treatment of cancer.

β-Particles are electrons emitted from the nucleus. They typically have a longer range in tissue (of the order of 1-5 mm) and are the most frequently used.

α-Particles are helium nuclei (two protons and two neutrons) that are emitted from the nucleus of a radioactive atom. Depending on their emission energy, they can travel 50-100 μm in tissue. They are positively charged and are orders of magnitude larger than electrons. The amount of energy deposited per path length travelled (designated ‘linear energy transfer’) of α-particles is approximately 400 times greater than that of electrons. This leads to substantially more damage along their path than that caused by electrons. An α-particle track leads to a preponderance of complex and largely irreparable DNA double-strand breaks. The absorbed dose required to achieve cytotoxicity relates to the number of α-particles traversing the cell nucleus. With use of this as a measure, cytotoxicity may be achieved with a range of 1 to 20α-particle traversals of the cell nucleus. The resulting high potency, combined with the short range of α-particles (which reduces normal organ toxicity), has led to substantial interest in developing α-particle-emitting agents. The α-particle emitters typically used include bismuth-212, lead-212, bismuth-213, actinium-225, radium-223 and thorium-227.

In some embodiments, Z′ is a diagnostic or therapeutic radionuclide.

### Representative Radionuclides

| Isotope | Radionuclide t_{1/2} (h) | Decay mode |
|---|---|---|
| ⁶⁰Cu | 0.4 | β+ (93%), EC (7%) |
| ⁶¹Cu | 3.3 | β+ (62%), EC (38%) |
| ⁶²Cu | 0.16 | β+ (98%), EC (2%) |
| ⁶⁴Cu | 12.7 | β+ (19%), EC (41%), β− (40%) |
| ⁶⁷Cu | 61.9 | |
| ⁶⁶Ga | 9.5 | β+ (56%), EC (44%) |
| ⁶⁷Ga | 78.2 | EC (100%) |
| ⁶⁸Ga | 1.1 | β+ (90%), EC (10%) |
| ⁴⁴Sc | 3.9 | β+ (94%), EC (6%) |
| ⁴⁷Sc | 80.2 | β− (100%) |
| ¹¹¹In | 67.2 | EC (100%) |
| ^{114m}In | 49.5 d | EC (100%) |
| ¹¹⁴In (daughter) | 73 s | β− (100%) |
| ¹⁷⁷Lu | 159.4 | β− (100%) |
| ⁸⁶Y | 14.7 | β+ (33%), EC (66%) |
| ⁹⁰Y | 64.1 | β− (100%) |
| ⁸⁹Zr | 78.5 | β+ (23%), EC (77%) |
| ²¹²Bi | 1.1 | α (36%), β− (64%) |
| ²¹³Bi | 0.76 | α (2.2%), β− (97.8%) |
| ²¹²Pb (daughter is ²¹²Bi) | 10.6 | β− (100%) |
| ²²⁵Ac | 240 | α (100%) |
| ²²⁷Th | 448.8 | α |
| ²¹¹At | 7.2 | α |

In some embodiments, Z′ is an Auger electron-emitting radionuclide. In some embodiments, Z′ is an Auger electron-emitting radionuclide that is 111-indium (¹¹¹ In), 67-gallium (⁶⁷Ga), 68gallium (⁶⁸Ga), 99m-technetium (^{99m}Tc), or 195m-platinum (^{195m}Pt).

In some embodiments, Z′ is an α-emitting radionuclide. In some embodiments, Z′ is an a-emitting radionuclide that is 225-actinium (²²⁵ Ac), 213-bismuth (²¹³Bi), 223-Radium (²²³Ra), or 212-lead (²¹²Pb).

In some embodiments, Z′ is an β-emitting radionuclide. In some embodiments, Z′ is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (6+Cu), 67-copper (⁶⁷Cu), 153-samarium (¹⁵³Sm), 89-strontium (89Sr), 198-gold (198 Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn).

In some embodiments, Z′ is a γ-emitting radionuclide. In some embodiments, Z′ is a γ-emitting radionuclide that is 60-cobalt (⁶⁰Co), 103-palldium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is an Auger electron-emitting radionuclide that is 111-indium (¹¹¹ In), 67-gallium (⁶⁷Ga), 68gallium (⁶⁸Ga), 99m-technetium (^{99m}Tc), or 195m-platinum (^{195m}Pt); or Z′ is an α-emitting radionuclide that is 225-actinium (²²⁵ Ac), 213-bismuth (²¹³Bi), 223-Radium (²²³Ra), or 212-lead (²¹²Pb); or Z′ is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (89Sr), 198-gold (198 Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); Z′ is a γ-emitting radionuclide that is 60-cobalt (6Co), 103-palldium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (89Sr), 198-gold (198 Au), 169-Erbium (¹⁶⁹Er), 165-dysprosium (¹⁶⁵Dy), or technetium-99m (^{99m}Tc).

In some embodiments, Z′ is ″+Tc, 90 In, 111 In, 67Ga, 68Ga, ⁸⁶Y, ⁹⁰Y, ¹⁷⁷Lu, ¹⁶¹Tb, ¹⁸⁶Re, ¹⁸⁸ Re, ⁶⁴Cu, ⁶⁷Cu, ⁵⁵Co, ⁵⁷Co, ⁴³Sc, ++Sc, ⁴⁷Sc, ²²⁵ Ac, ²¹³Bi, ²¹²Bi, ²¹²Pb, ²²⁷Th, ¹⁵³Sm, ¹⁶⁶Ho, ¹⁵²Gd, ¹⁵³Gd, ¹⁵⁷Gd, and ¹⁶⁶Dy.

In some embodiments, Z′ is ⁶⁷Cu, ⁶⁴Cu, ⁹⁰Y, ¹⁰⁹Pd, ¹¹¹ Ag, ¹⁴⁹Pm, ¹⁵³Sm, ¹⁶⁶Ho, ⁹⁹mTc, ⁶⁷Ga, ⁶⁸Ga, ¹¹¹ In, ⁹⁰Y, ¹⁷⁷Lu, ¹⁸⁶Re, ¹⁸⁸Re, ¹⁹⁷ Au, ¹⁹⁸ Au, ¹⁹⁹ Au, ¹⁰⁵Rh, ¹⁶⁵Ho, ¹⁶¹Tb, ¹⁴⁹Pm, ++Sc, ⁴⁷Sc, ⁷⁰ As, ⁷¹ As, ⁷² As, ⁷³ As, ⁷⁴As, ⁷⁶ As, ⁷⁷As, ²¹²Pb, ²¹²Bi, ²¹³Bi, ²²⁵ Ac, ¹¹⁷mSn, ⁶⁷Ga, ²⁰¹T^{1, 160}Gd, ¹⁴⁸Nd, and ⁸⁹Sr.

In some embodiments, Z′ is ⁶⁸Ga, +³Sc, ″Sc, ⁴⁷Sc, ¹⁷Lu, ¹⁶¹Tb, ²⁵ Ac, ²¹³Bi, ²¹²Bi, or ²¹²Pb. In some embodiments, Z′ is ⁶⁷Ga, ⁹⁹mTc, ¹¹¹ In, or ²⁰¹T^{1.}

### Exemplary Chelator and Radionuclide Complexes

Radionuclides have useful emission properties that can be used for diagnostic imaging techniques, such as single photon emission computed tomography (SPECT, e.g. ⁶⁷Ga, ^{99m}Tc, ¹¹¹ In, ¹⁷⁷Lu) and positron emission tomography (PET, e.g. ⁶⁸Ga, ⁶⁴Cu, ++Sc, ⁸⁶Y, ⁸⁹Zr), as well as therapeutic applications (e.g. ⁴⁷Sc, ¹¹+mIn, ¹⁷⁷Lu, ⁹⁰Y, ^{212/213}Bi, ²¹²Pb, ²²⁵ Ac, ^{186/188}Re). A fundamental component of a radiometal-based radiopharmaceutical is the chelator, the ligand system that binds the radiometal ion in a tight stable coordination complex so that it can be properly directed to a desirable molecular target in vivo. Guidance for selecting the optimal match between chelator and radiometal for a particular use is provided in the art (e.g., see Price et al., “Matching chelators to radiometals for radiopharmaceuticals”, Chem. Soc. Rev., 2014, 43, 260-290).

In some embodiments, R⁶ is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py₄pa; Hapy4pa-benzyl; H₄octapa; H₄octapa-benzyl; and TTHA; or a radionuclide complex thereof.

In some embodiments. R⁶ is:

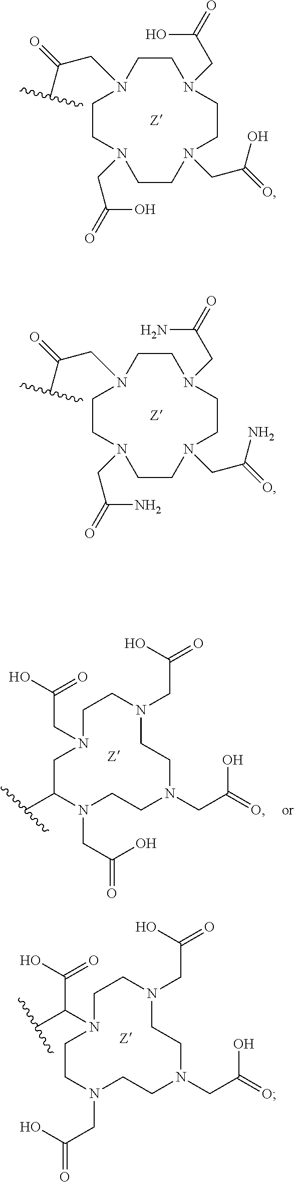

wherein Z′ is a diagnostic or therapeutic radionuclide.

In some embodiments, the radionuclide (Z′) is 111-indium (¹¹¹ In), 115-indium (¹¹⁵ In), 67-gallium (⁶⁷Ga), 68-gallium (⁶⁸Ga), 70-gallium (⁷⁰Ga), 225-actinium (²²⁵Ac), 175-lutetium (¹⁷⁵Lu) or 177-lutetium (¹⁷⁷Lu).

In some embodiments, the radionuclide (Z′) is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸ Au), 169-Erbium (¹⁶⁹Er), 165-dysprosium (¹⁶⁵Dy), or technetium-99m (^{99m}Tc).

### Emission Tomography

In some embodiments, R⁶ comprises a chelated radionuclide that is suitable for positron emission tomography (PET) analysis or single-photon emission computerized tomography (SPECT). In some embodiments, R⁶ comprises a chelated radionuclide that is suitable for single-photon emission computerized tomography (SPECT). In some embodiments, R⁶ comprises a chelated radionuclide that is suitable for positron emission tomography (PET) analysis. In some embodiments, R⁶ comprises a chelated radionuclide that is suitable for positron emission tomography imaging, positron emission tomography with computed tomography imaging, or positron emission tomography with magnetic resonance imaging (MRI).

In some embodiments, R⁶ is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py₄pa; H₄py₄pa-benzyl; H₄octapa; H₄octapa-benzyl; and TTHA; or a radionuclide complex thereof. In some embodiments, the radionuclide is copper-64 (6+Cu), gallium-68 (⁶⁸Ga), or technetium-99m (^{99m}Tc).

In some embodiments, a conjugate described herein is designed to have a prescribed elimination profile. The elimination profile can be designed by adjusting the sequence and length of the non-peptide ligand, the property of the linker, the type of radionuclide, etc. In some embodiments, the conjugate has an elimination half-life of about 5 minutes to about 12 hours. In some embodiments, the conjugate has an elimination half-life of about 10 minutes to about 8 hours. In some embodiments, the conjugate has an elimination half-life of at least about 15 minutes, at least about 30 minutes, at least about 1 hour, at least about 2 hours, at least about 3 hours, at least about 4 hours, at least about 5 hours, at least about 6 hours, at least about 8 hours. In some embodiments, the conjugate has an elimination half-life of at most about 15 minutes, at most about 30 minutes, at most about 1 hour, at most about 2 hours, at most about 3 hours, at most about 4 hours, at most about 5 hours, at most about 6 hours, or at most about 8 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 15 minutes to about 1 day. In some embodiments, the elimination half-life of the conjugate in a tumor is at least 1.1, at least 1.2, at least 1.3, at least 1.4, at least 1.5, at least 2.0, at least 2.5, at least 3.0, at least 4.0, or at least 5.0-fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the pharmaceutical in the living system. In some embodiments, the elimination half-life is measured as effective half-life, which is the half-life of a radiopharmaceutical in a living system taking into account the half-life of the radionuclide.

Response and toxicity prediction is essential for the rational implementation of cancer therapy. The biological effects of radionuclide therapy are mediated by a well-defined physical quantity, the absorbed dose (D), which is defined as the energy absorbed per unit mass of tissue.

Radiation dosimetry is the measurement, calculation and assessment of the ionizing radiation dose absorbed by an object, usually the human body, and may be thought of as the ability to perform the equivalent of a pharmacodynamic study in treated patients in real time. This applies both internally, due to ingested or inhaled radioactive substances, or externally due to irradiation by sources of radiation. Dosimetry analysis may be performed as part of patient treatment to calculate tumor versus normal organ absorbed dose and therefore the likelihood of treatment success.

A conjugate described herein can have a prescribed time-integrated activity coefficient (i.e., ã) in a tumor or non-tumor tissues of a subject. As used herein, ã represents the cumulative number of nuclear transformations occurring in a source tissue over a dose-integration period per unit administered activity. The a value of a conjugate can be tuned by modifications of the NPDC. The ã value can be determined using a method known in the art. In some embodiments, the ã value of the conjugate in a tumor is from about 10 minutes to about 1 day. The ã value of the conjugate in a tumor can be the same as the a value of the conjugate in a non-tumor tissue of the subject. The ã value of the conjugate in a tumor can be longer or shorter than the ã value of the conjugate in a non-tumor tissue of the subject. In some embodiments, the a value of the conjugate in a tumor is at least 1.1, at least 1.2, at least 1.3, at least 1.4, at least 1.5, at least 2.0, at least 2.5, at least 3.0, at least 4.0, or at least 5.0-fold of the a value of the conjugate in a non-tumor tissue of the subject.

A conjugate described herein can have an ã value in an organ of a subject. In some embodiments, the conjugate has an ã value in a kidney of the subject of at most 24 hours. In some embodiments, the ã value of the conjugate in a kidney of the subject is at most 18 hours, 15 hours, 12 hours, 10 hours, 8 hours, 6 hours, or 5 hours. In some embodiments, the a value of the conjugate in a kidney of the subject is about 30 minutes to about 24 hours. In some embodiments, the ã value of the conjugate in a kidney of the subject is about 2 to 24 hours. In some embodiments, the a value of the conjugate in a kidney of the subject is more than 24 hours. In some embodiments, the ã value of the conjugate in a liver of the subject is at most 24 hours. In some embodiments, the ã value of the conjugate in a liver of the subject is at most 18 hours, 15 hours, 12 hours, 10 hours, 8 hours, 6 hours, or 5 hours. In some embodiments, the a value of the conjugate in a liver of the subject is about 30 minutes to about 24 hours. In some embodiments, the ã value of the conjugate in a liver of the subject is about 2 to 24 hours. In some embodiments, the a value of the conjugate in a liver of the subject is more than 24 hours.

### Linkers

In some embodiments, the linker has a prescribed length thereby linking the follicle-stimulating hormone receptor (FSHR) targeting ligand and the chelating moiety or a radionuclide complex thereof (R⁶)_{w}hile allowing an appropriate distance therebetween.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, the cleavable linker comprises one or more of substituted or unsubstituted alkylene, substituted or unsubstituted cycloalkylene, substituted or unsubstituted heterocycloalkylene, substituted or unsubstituted arylene, and substituted or unsubstituted heteroarylene.

In some embodiments, the linker comprises a click chemistry residue. In some embodiments, the linker is attached to a non-peptide ligand, to a metal chelator or both via click chemistry. For example, in some embodiments, a non-peptide ligand comprises an azide group that reacts with an alkyne moiety of the linker. For another example, in some embodiments, a non-peptide ligand comprises an alkyne group that reacts with an azide of the linker. The metal chelator and the linker can be attached similarly. In some embodiments, the linker comprises an azide moiety, an alkyne moiety, or both. In some embodiments, the linker comprises a triazole moiety.

In some embodiments, L¹ is -L²-, -L³-, -L⁴-, -L⁵-, -L⁶-, -L⁷-, -L²-L³-, -L²-L⁴-, -L²-L⁷-L⁵-L⁶-L⁷-, or a combination thereof; L² is absent, substituted or unsubstituted C₁-C₂₀alkylene, substituted or unsubstituted C₁-C₂₀alkylene-NR¹³—, substituted or unsubstituted C₁-C₂₀alkylene-C(═O)—, substituted or unsubstituted C₁-C₂₀alkylene-C(═O)NH—, substituted or unsubstituted C₁-C₂₀alkylene-NR¹³C(═O)—, substituted or unsubstituted 2 to 20 membered heteroalkylene, (CH₂CH₂O), —, —(OCH₂CH₂), —, —(CH₂CH₂O)_{w}—CH₂CH₂—, —CH₂CH₂NR¹³—(CH₂CH₂O)_{w}—, (CH₂CH₂O)_{w}—CH₂CH₂NR¹³—, —CH₂CH₂NR¹³C(═O)—(CH₂CH₂O)_{w}, —(CH₂CH₂O)_{w}—CH₂CH₂NR¹³C(═O)—, —CH₂CH₂C(═O)NR¹³—(CH₂CH₂O)—, —CH₂CH₂NR¹³C(═O)CH₂—(OCH₂CH₂)_{w} or —(CH₂CH₂O)—CH₂CH₂C(═O)NH—; w is 1, 2, 3, 4, 5, or 6; z is 1, 2, 3, 4, 5, 6, 7, 8, 9, or 10; L³ is absent, a natural or unnatural amino acid or peptide that is formed from two or more independently selected natural and unnatural amino acids, wherein when two or more amino acids are present then the N atom of the amide linking the amino acids is optionally substituted with C₁-C₆ alkyl; L⁴ is absent, substituted or unsubstituted 2 to 10 membered heteroalkylene, —CH₂—(OCH₂CH₂)—, —(CH₂CH₂O)—CH₂CH₂—, —(CH₂CH₂O)_{w}CH₂CH₂NR¹⁴C(═O)(CH₂CH₂O)_{w}CH₂CH₂—, —(CH₂CH₂O)_{w}CH₂CH₂C(═O)NR¹+ (CH₂CH₂O)ᵥCH₂CH₂—, —C(—O)CH₂CH₂, —CH₂CH₂C(═O)—, or C₁-C₆ alkylene that is optionally substituted with 1 or 2 groups independently selected from —OH, —NR¹⁴₂, —CO₂R¹⁴, —O(CH₂CH₂O)ᵤ—CH₃, —NR¹+ (CH₂CH₂O)ᵤ—CH₃, —NR¹⁴C(═O)(CH₂CH₂O)ᵤ—CH₃, or —CH₂OCH₂CH₂CO₂R¹⁴; each instance of v is independently 1, 2, 3, 4, 5, or 6; u is 1, 2, 3, 4, 5, or 6; L⁵ is absent, —O—, —S—, —S(═O)—, —S(═O)₂, —NR¹⁵—, —CH(═NH)—, —CH(═N—NH)—, —CCH₃(═NH)—, —CCH₃ (═N—NH)—, —C(═O)NR¹⁵—, —NR¹⁵C(═O), —NR¹⁵C(═O)O—, —NR¹⁵C(═O)NR¹⁵—, or —OC(═O)NR¹⁵—; L⁶ is absent or -L⁸-L⁹-L¹⁰-; L⁸ is absent, —(CH₂)ᵥ—, —NR^{w}—, —NR^{w}—(CH₂)ᵥ—, —(CH₂)ₜ—C(═O)—, —C(═O)—(CH₂)—, —(CH₂)—NR^{w}—, —(CH₂)—NR^{w}C(═O)—, —(CH₂)ₜ—C(═O)NR^{w}—, —CH(NHR^{w})—(CH₂)ₜ—C(═O)—, —NR^{w}C(═O)—(CH₂)—, and —C(═O)NR^{w}—(CH₂)ᵥ—; t is 0, 1, 2, or 3; L¹⁰ is absent, —(CH₂)ᵣ—, —NR^{w}—, —NR^{w}—(CH₂)ᵣ—, —(CH₂)ᵣ—C(═O)—, —C(═O)—(CH₂)ᵣ—, —(CH₂), —NR^{w}—, —(CH₂)ᵣ—NR^{w}C(═O)—, —(CH₂)ᵣ—C(═O)NR^{w}—, —CH(NHR^{w})—(CH₂)—C(═O)—, —NR^{w}C(═O)—(CH₂)ᵣ—, and —C(═O)NR^{w}—(CH₂)ᵥ—; r is 0, 1, 2, or 3; each R^{w} is independently selected from H, C₁-C₆ alkyl, C₁-C₆ alkyl-CO₂H, —(CH₂CH₂O)ₛ—CH₃, —C(═O)—(CH₂CH₂O)ₛ—CH₃, or —(CH₂CH₂O)ₛ—CH₂CH₂CO₂H; s is 1, 2, 3, 4, 5, or 6; L¹ is substituted or unsubstituted cycloalkylene, substituted or unsubstituted heterocycloalkylene, substituted or unsubstituted arylene, or substituted or unsubstituted heteroarylene; each R¹³, R¹⁴, and R¹⁵ are independently selected from H or unsubstituted C₁-C₄ alkyl; and L⁷ is absent, —NH—, —N(CH₃)—, —O—NH—, or substituted or unsubstituted N-heterocycloalkylene, or —O—NH═(substituted or unsubstituted N-heterocycloalkylene).

In some embodiments, L¹ is -L²-, -L³-, -L⁴-, -L⁵-, -L⁶-, -L⁷-, -L²-L³-L²-L⁴-, -L²-L⁶--L²-L⁷-, -L⁴-L⁶-, - L⁴-L⁷-, -L⁶-L⁷-, -L²-L⁴-L⁷-, -L²-L⁵-L⁷-, -L²-L⁶-L⁷-, -L³-L⁴-L⁷-, -L³-L⁵-L⁷-, or -L²-L³-L⁴-L⁵-L⁶-L⁷-; or a combination thereof; L² is absent, substituted or unsubstituted C₁-C₂₀ alkylene, substituted or unsubstituted C₁-C₂₀ alkylene-NH—, substituted or unsubstituted C₁-C₂₀ alkylene-C(═O)—, substituted or unsubstituted C₁-C₂₀ alkylene-C(═O)NH—, substituted or unsubstituted C₁-C₂₀ alkylene-NHC(═O)—, substituted or unsubstituted 2 to 20 membered heteroalkylene, —(CH₂CH₂O), —, —(OCH₂CH₂), —(CH₂CH₂O)_{w}—CH₂CH₂—, —CH₂CH₂NH—(CH₂CH₂O)_{w}—, —(CH₂CH₂O)_{w}—CH₂CH₂NH—, —CH₂CH₂NHC(═O)—(CH₂CH₂O)_{w}, —(CH₂CH₂O)ₛ—CH₂CH₂NHC(═O)—, —CH₂CH₂C(═O)NH—(CH₂CH₂O)ₜ—, —CH₂CH₂NHC(═O)CH₂—(OCH₂CH₂)_{w} or (CH₂CH₂O)ₛ—CH₂CH₂C(═O)NH—; L³ is absent, a natural or unnatural amino acid or peptide that is formed from one or more independently selected natural and unnatural amino acids, wherein when two or more amino acids are present then the N atom of the amide linking the amino acids is optionally substituted with C₁-C₆ alkyl; L⁴ is —CH₂—(OCH₂CH₂)—, —(CH₂CH₂O)—CH₂CH₂—, (CH₂CH₂O)_{w}CH₂CH₂NHC(═O)(CH₂CH₂O)_{w}CH₂CH₂—, —(CH₂CH₂O)CH₂CH₂C(═O)NH(CH₂CH₂O)_{w}CH₂CH₂—, —C(═O)CH₂CH₂, —CH₂CH₂C(═O)—, or C₁-C₆ alkylene that is optionally substituted with 1 or 2 groups independently selected from —OH, —NH₂, —CO₂H, —O(CH₂CH₂O)ᵤ—CH₃, —NH(CH₂CH₂O)ᵤ—CH₃, —NHC(═O)(CH₂CH₂O)ᵤ—CH₃, and —CH₂OCH₂CH₂CO₂H; L⁵ is absent, —C(═O)NH—, or —NHC(—O)—; L⁶ is absent or -L⁸-L⁹-L¹⁰-; L⁸ is absent, —(CH₂)ₜ—, —NR^{w}—(CH₂)—, —(CH₂)ₜ—C(═O)—, —C(═O)—(CH₂)ᵥ—, —(CH₂), —NR^{w}—, —(CH₂), —NR^{w}C(═O)—, —(CH₂)—C(═O)NR^{w}—, —CH(NHR^{w})—(CH₂)ₜ—C(═O)—, —NR^{w}C(═O)—(CH₂)ᵥ—, and —C(═O)NR^{w}—(CH₂)ₜ—; L¹⁰ is absent or —(CH₂)ᵣ—; r is 0, 1, 2, or 3; each R^{w} is independently selected from H, C₁-C₆ alkyl, C₁-C₆ alkylCO₂H, —(CH₂CH₂O)ₛ—CH₃, —C(—O)—(CH₂CH₂O)ₛ—CH₃, or (CH₂CH₂O)ₛ—CH₂CH₂CO₂H; L¹ is substituted or unsubstituted cycloalkylene, substituted or unsubstituted heterocycloalkylene, substituted or unsubstituted arylene, or substituted or unsubstituted heteroarylene; and L¹ is absent, —NH—, —N(CH₃)—, —O—NH—, substituted or unsubstituted N-heterocycloalkylene, or —O—NH═(substituted or unsubstituted N-heterocycloalkylene).

In some embodiments, L¹ is -L²- or -L²-L⁷-In some embodiments, L¹ is -L²-. In some embodiments, L¹ is -L²-L⁷

In some embodiments, L¹ is -L³- or -L²-L³-In some embodiments, L¹ is -L³-. In some embodiments, L¹ is -L²-L³_

In some embodiments, L² is substituted or unsubstituted C₁-C₆ alkylene-C(═O)—and L³ is a peptide that is formed from one or more independently selected natural and unnatural amino acids.

In some embodiments, L² is absent. In some embodiments, L² is —CH₂— or —CH₂CH₂—. In some embodiments, L² is —CH₂—. In some embodiments, L² is substituted or unsubstituted C₁-C₂₀alkylene-NH—, substituted or unsubstituted C₁-C₂₀alkylene-C(═O)—, substituted or unsubstituted C₁-C₂₀alkylene-C(═O)NH—, or substituted or unsubstituted C₁-C₂₀alkylene-NHC(═O)—. In some embodiments, L² is —(CH₂CH₂O)_{w}—CH₂CH₂—, —(CH₂CH₂O)_{w}—CH₂CH₂NH—, —CH₂CH₂NHC(═O)—(CH₂CH₂O)_{w}—, —(CH₂CH₂O)_{w}—CH₂CH₂NHC(═O)—, —CH₂CH₂C(═O)NH—(CH₂CH₂O)_{w}—, or —CH₂CH₂NHC(═O)CH₂—(OCH₂CH₂)_{w}. In some embodiments, —(CH₂CH₂O)_{w}—CH₂CH₂— or —(CH₂CH₂O)_{w}—CH₂CH₂NH—. In some embodiments, L² is substituted or unsubstituted C₁-C₂₀alkylene-NH—. In some embodiments, L² is substituted or unsubstituted C₁-C₂₀alkylene-C(═O)—. In some embodiments, L² is substituted or unsubstituted C₁-C₂₀alkylene-C(—O)NH—. In some embodiments, L² is or substituted or unsubstituted C₁-C₂₀alkylene-NHC(═O)—. In some embodiments, L² is —(CH₂CH₂O)_{w}—CH₂CH₂—. In some embodiments, L² is —(CH₂CH₂O)_{w}—CH₂CH₂NH—. In some embodiments, L² is —CH₂CH₂NHC(—O)—(CH₂CH₂O) . . . . In some embodiments, L² is —(CH₂CH₂O)_{w}—CH₂CH₂NHC(═O)—. In some embodiments, L² is —CH₂CH₂C(═O)NH—(CH₂CH₂O)_{w}—. In some embodiments, L² is —CH₂CH₂NHC(═O)CH₂—(OCH₂CH₂)_{w}—.

In some embodiments, L³ is absent. In some embodiments, L³ is a natural amino acid, an unnatural amino acid, or peptide that is formed from two or more independently selected amino acids selected from the group consisting of alanine (Ala), arginine (Arg), asparagine (Asn), aspartate (Asp), cysteine (Cys), cysteic acid, glutamine (Gln), glutamate (Glu), glycine (Gly), leucine (Leu), lysine (Lys), methionine (Met), phenylalanine (Phe), proline (Pro), serine (Ser), sarcosine, tyrosine (Tyr), and valine (Val), wherein when two or more amino acids are present then the N atom of the amide linking the amino acids is optionally substituted with —CH₃. In some embodiments, the peptide is formed from one or more independently selected L-amino acids. In some embodiments, the peptide is formed from one or more independently selected D-amino acids. In some embodiments, the peptide is formed from one or more independently selected L-amino acids and one or more independently selected D-amino acids.

In some embodiments, L³ is cysteic acid. In some embodiments, L³ is aspartic acid. In some embodiments, L³ is glutamic acid. In some embodiments, L³ is sarcosine. In some embodiments, L³ is di-sarcosine. In some embodiments, L³ is penta-sarcosine.

In some embodiments, L³ is a peptide that is: Ala-Lys-Ser-Asn-Asn-Ser-Ala-NH₂, Ala-Ser-Asn-Lys-Asn-Ser-Ala-NH₂, Ala-Ser-Asn-Asn-Ser-Lys-Ala-NH₂, Ala-Arg-Arg-Lys-Glu-Glu-Glu-NH₂, or Ala-Glu-Ala-Lys-Glu-Ala-NH₂. In some embodiments, L³ is a peptide that is: Ala-Lys-Ser-Asn-Asn-Ser-Ala-NH₂. In some embodiments, L³ is a peptide that is: Ala-Ser-Asn-Lys-Asn-Ser-Ala-NH₂. In some embodiments, L³ is a peptide that is: Ala-Ser-Asn-Asn-Ser-Lys-Ala-NH₂. In some embodiments, L³ is a peptide that is: Ala-Arg-Arg-Lys-Glu-Glu-Glu-NH₂. In some embodiments, L³ is a peptide that is: Ala-Glu-Ala-Lys-Glu-Ala-NH₂.

In some embodiments, L⁴ is absent. In some embodiments, L⁴ is substituted or unsubstituted 2 to 10 membered heteroalkylene. In some embodiments, L⁴ is —CH₂—(OCH₂CH₂)—. In some embodiments, L⁴ is —(CH₂CH₂O), —CH₂CH₂—. In some embodiments, L⁴ is —(CH₂CH₂O)_{w}CH₂CH₂NHC(═O)(CH₂CH₂O)_{w}CH₂CH₂—, In some embodiments, L⁴ is —(CH₂CH₂O)ᵥCH₂CH₂C(═O)NH(CH₂CH₂O)_{w}CH₂CH₂—. In some embodiments, L⁴ is —C(═O)CH₂CH₂. In some embodiments, L⁴ is —CH₂CH₂C(═O)—. In some embodiments, L⁴ is C₁-C₆ alkylene that is optionally substituted with 1 or 2 groups independently selected from —OH, —NH₂, —CO₂H, —O(CH₂CH₂O)ᵤ—CH₃, —NH(CH₂CH₂O)ᵤ—CH₃, —NHC(═O)(CH₂CH₂O)ᵤ—CH₃, or —CH₂OCH₂CH₂CO₂H. In some embodiments, L⁴ is C₁-C₆ alkylene that is optionally substituted with 1 or 2 groups independently selected from —OH, —NH₂, —CO₂H, or —CH₂OCH₂CH₂CO₂H. In some embodiments, L⁴ is unsubstituted C₁-C₆ alkylene.

In some embodiments, L⁵ is absent. In some embodiments, L⁵ is —O—. In some embodiments, L⁵ is —S—. In some embodiments, L⁵ is —S(═O)—. In some embodiments, L⁵ is —S(═O)₂. In some embodiments, L⁵ is —NH—. In some embodiments, L⁵ is —CH(═NH)—. In some embodiments, L⁵ is —CH(═N—NH)—. In some embodiments, L⁵ is —CCH₃(═NH)—. In some embodiments, L⁵ is —CCH₃ (═N—NH)—. In some embodiment, L⁵ is —C(—O)NH— or —NHC(═O)—. In some embodiments, L⁵ is —C(—O)NH—. In some embodiments, L⁵ is —NHC(═O)—. In some embodiments, L⁵ is —NHC(—O)O—. In some embodiments, L⁵ is —NHC(═O)NH—. In some embodiments, L⁵ is —OC(═O)NH—.

In some embodiments, L¹ is a substituted or unsubstituted cycloalkylene. In some embodiments, L¹ is a substituted or unsubstituted C₄-C₈ cycloalkylene. In some embodiments, L¹ is a substituted or unsubstituted heterocycloalkylene, In some embodiments, L¹ is a substituted or unsubstituted 3 to 8 membered heterocycloalkylene. In some embodiments, L⁹ is azetidinylene, pyrrolidinylene, piperidinylene or piperazinylene. In some embodiments, L⁹ is a monosaccharide. In some embodiments, L⁹ is

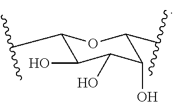 [SMILES: *C1OC(*)C(O)C(O)C1O]

In some embodiments, L⁹ is a 7 to 12 membered spirocyclic heterocycloalkylene. In some embodiments, L⁹ is

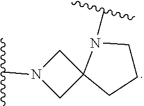 [SMILES: *N1CC2(CCCN2*)C1]

In some embodiments, L⁹ is

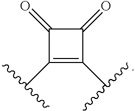 [SMILES: *c1c(*)c(=O)c1=O]

In some embodiments, L⁹ is a substituted or unsubstituted arylene. In some embodiments, L⁹ is phenylene. In some embodiments, L⁹ is

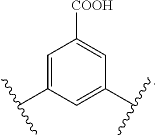 [SMILES: *c1cc(*)cc(C(=O)O)c1]

In some embodiments, L⁹ is a substituted or unsubstituted heteroarylene.

In some embodiments, L⁸ is absent. In some embodiments, L⁸ is —(CH₂)—, —(CH₂)ₜ—C(═O)NR^{w}—, or —CH(NHR^{w})—(CH₂)ₜ—C(═O)—. In some embodiments, L⁸ is —(CH₂)—. In some embodiments, L⁸ is —NR^{w}—. In some embodiments, L⁸ is —NR^{w}—(CH₂)ᵥ—. In some embodiments, L⁸ is —(CH₂)ₜ—C(═O)—. In some embodiments, L⁸ is —C(═O)—(CH₂)—. In some embodiments, L⁸ is —(CH₂)ₜ—NR^{w}—. In some embodiments, L⁸ is —(CH₂)ₜ—NR^{w}C(═O)—. In some embodiments, L⁸ is —(CH₂)ₜ—C(═O)NR^{w}—. In some embodiments, L⁸ is —CH(NHR^{w})—(CH₂)ᵣ—C(═O)—. In some embodiments, L⁸ is —NR^{w}C(—O)—(CH₂)—. In some embodiments, L⁸ is —C(═O)NR^{w}—(CH₂)ₜ—.

In some embodiments, L¹⁰ is absent, —(CH₂)ᵣ, —NR^{w}—(CH₂)ᵣ, or —C(—O)—(CH₂)ᵣ—. In some embodiments, L¹⁰ is absent or —(CH₂)ᵣ—. In some embodiments, L¹⁰ is absent. In some embodiments, L¹⁰ is —(CH₂)ᵣ—. In some embodiments, L¹⁰ is —NR^{w}—. In some embodiments, L¹⁰ is —NR^{w}—(CH₂)ᵣ—. In some embodiments, L¹⁰ is —(CH₂)ᵣ—C(═O)—. In some embodiments, L¹⁰ is —C(—O)—(CH₂) . . . . In some embodiments, L¹⁰ is —(CH₂)ᵣ—NR^{w}—. In some embodiments, L¹⁰ is —(CH₂)ᵣ—NR^{w}C(═O)—. In some embodiments, L¹⁰ is —(CH₂)—C(═O)NR^{w}—. In some embodiments, L¹⁰ is —CH(NHR^{w})—(CH₂)ᵣ—C(═O)—. In some embodiments, L¹⁰ is —NR^{w}C(═O)—(CH₂)ᵣ. In some embodiments, L¹⁰ is —C(═O)NR^{w}—(CH₂)ᵣ.

In some embodiments, L⁶ is absent. In some embodiments, L⁶ is -L⁸-L⁹-L¹⁰-.

In some embodiments, L⁷ is absent. In some embodiments, L⁷ is —NH—. In some embodiments, L⁷ is —N(CH₃)—. In some embodiments, L⁷ is —O—NH—. In some embodiments, L⁷ is substituted or unsubstituted N-heterocycloalkylene. In some embodiments, L⁷ is substituted or unsubstituted N-heterocycloalkylene, wherein the N-heterocycloalkylene is bonded to adjacent groups (e.g., -L⁶- or —R⁶) via a nitrogen atom of the N-heterocycloalkylene

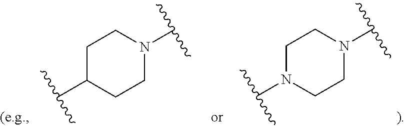 [SMILES: *C1CCN(*)CC1 ; *N1CCN(*)CC1]

In some embodiments, L⁷ is unsubstituted N-heterocycloalkylene. In some embodiments, L⁷ is

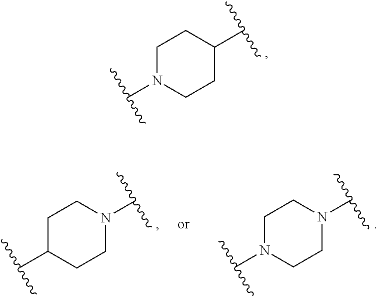 [SMILES: *C1CCN(*)CC1 ; *C1CCN(*)CC1 ; *N1CCN(*)CC1]

In some embodiments, L⁷ is

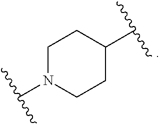 [SMILES: *C1CCN(*)CC1]

In some embodiments, L⁷ is

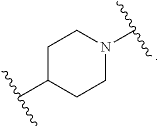 [SMILES: *C1CCN(*)CC1]

In some embodiments, L⁷ is

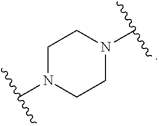 [SMILES: *N1CCN(*)CC1]

In some embodiments, L¹ is -L²-; L² is —(CH₂CH₂O)_{w}—CH₂CH₂NH; and w is 1, 2, 3, 4, 5, or 6. In some embodiments, L¹ is -L²; L² is —(CH₂CH₂O)_{w}—CH₂CH₂NH; and w is 1, 2, 3, 4, 5, or 6.

In some embodiments, L¹ is -L³-; and L³ is a natural amino acid, an unnatural amino acid, or peptide.

In some embodiments, L¹ is -L⁶-; L⁶ is -L⁸-L⁹-L¹⁰; L⁸ is —(CH₂)—C(═O)NR^{w}—; L¹ is substituted or unsubstituted heterocycloalkylene; L¹⁰ is absent; and t is 1, 2, or 3. In some embodiments, L¹ is azetidine, pyrrolidine, piperidine, or piperazine. In some embodiments, R^{w} is C₁-C₆ alkyl-CO₂H. In some embodiments, R^{w is —(CH}₂CH₂O)ₛ—CH₂CH₂CO₂H.

In some embodiments, L¹ is -L⁷-; and L⁷ is substituted or unsubstituted N-heterocycloalkylene.

In some embodiments, L¹ is -L²-L³-; L² is substituted or unsubstituted C₁-C₆ alkylene-NH—, substituted or unsubstituted C₁-C₆ alkylene-C(═O)—, substituted or unsubstituted C₁-C₆ alkylene-NH—, or —(CH₂CH₂O)_{w}—CH₂CH₂NH—; and L³ is a natural or unnatural amino acid or peptide. In some embodiments, L³ is a peptide.

In some embodiments, L¹ is -L²-L⁴-; L² is —(CH₂CH₂O)_{w}—CH₂CH₂NHC(═O)—; and L⁴ is unsubstituted C₁-C₆ alkylene.

In some embodiments, L¹ is -L²-L⁷; L² is substituted or unsubstituted C₁-C₆ alkylene-, substituted or unsubstituted C₁-C₆ alkylene-C(═O)NH—, —(CH₂CH₂O)—CH₂CH₂—; and L⁷ is —NH—, —O—NH—, or substituted or unsubstituted N-heterocycloalkylene.

In some embodiments, L¹ is -L²-L⁷; L² is —(CH₂CH₂O)_{w}—CH₂CH₂—; and L⁷ is —NH—.

In some embodiments, L¹ is -L⁴-L⁶-; L⁴ is C₁-C₆ alkylene that is optionally substituted with 1 or 2 groups independently selected from —OH or —NH₂; L⁶ is -L⁸-L⁹-L¹⁰-; L⁸ is —(CH₂)ₜ—C(═O)NR^{w}—; R^{w} is H; L¹ is substituted or unsubstituted heterocycloalkylene; and L¹⁰ is absent.

In some embodiments, L¹ is -L⁴-L⁷; L⁴ is C₁-C₆ alkylene that is optionally substituted with 1 or 2 groups independently selected from —OH or —NH₂; and L⁷ is —NH—.

In some embodiments, L¹ is -L⁶-L⁷-; L⁶ is -L⁸-L⁹-L¹⁰; L⁸ is absent, —CH(NHR^{w})—(CH₂)ₜ—C(═O)—, or —(CH₂)—C(═O)NR^{w}—; L¹ is substituted or unsubstituted heterocycloalkylene; L¹⁰ is absent, —(CH₂)ᵣ—, or —C(═O)—(CH₂)ᵣ—; and L¹ is —NH— or substituted or unsubstituted N-heterocycloalkylene. In some embodiments, R^{w is —C(═O)—(CH}₂CH₂O)ₛ—CH₃ or —(CH₂CH₂O)ₛ—CH₂CH₂CO₂H.

In some embodiments, L¹ is -L²-L⁴-L⁷, L² is substituted or unsubstituted C₁-C₆ alkylene-NHC(═O)—, —(CH₂CH₂O), —, or —CH₂CH₂NHC(═O)—(CH₂CH₂O)_{w}; L⁴ is —CH₂—(OCH₂CH₂)— or C₁-C₆ alkylene that is optionally substituted with 1 or 2 groups independently selected from —OH, —NH₂, or —CH₂OCH₂CH₂CO₂H; and L⁷ is —NH— or substituted or unsubstituted N-heterocycloalkylene.

In some embodiments, L¹ is -L²-L⁷-; L² is substituted or unsubstituted C₁-C₆ alkylene; and L⁷ is —O—N═ (substituted or unsubstituted N-heterocycloalkylene).

In some embodiments, L¹ is -L²-L⁶-L⁷; L² is substituted or unsubstituted C₁-C₆ alkylene-NH—, substituted or unsubstituted C₁-C₆ alkylene-NHC(═O)—, substituted or unsubstituted C₁-C₆ alkylene-C(═O)NH, or —(CH₂CH₂O)_{w}—CH₂CH₂NHC(═O)—; L⁶ is -L⁸-L⁹-L¹⁰-; L⁸ is absent or —(CH₂)ᵣ; L⁹ is substituted or unsubstituted heterocycloalkylene; L¹⁰ is absent, —(CH₂)ᵣ—, or —NR^{w}—(CH₂)—; and L⁷ is —NH—.

In some embodiments, L¹ is L³-L⁴-L⁷-; L³ is a peptide, wherein the N atom of the amide linking the amino acids is substituted with a —CH₃; L⁴ is —C(═O)CH₂CH₂—; and L⁷ is —NH—.

In some embodiments, L¹ is L⁴-L⁵-L⁷; L⁴ is a C₁-C₆ alkylene that is optionally substituted with 1 or 2 groups independently selected from —OH or —NH₂; L⁵ is —C(—O)NH—; and L⁷ is substituted or unsubstituted N-heterocycloalkylene. In some embodiments, R is —(CH₂CH₂O)ᵤ—CH₂CH₂N(L¹-R⁶)₂—. In some embodiments, R is —CH-(L¹R⁶)₂. In some embodiments, L¹ is L²; L² is —(CH₂CH₂O)_{w}—CH₂CH₂NH—; and w is 3. In some embodiments, L¹ is L²-L⁴-L⁷; L² is substituted or unsubstituted C₁-C₂₀alkylene-NHC(═O)—; L⁴ is C₁-C₆ alkylene; and L¹ is NH.

In some embodiments, Z is —C(═O)NH—; L¹ is -L²-; L² is —(CH₂CH₂O)_{w}—CH₂CH₂NH; and w is 2. In some embodiments, Z is —C(═O)NH—; L¹ is -L²-; L² is —(CH₂CH₂O)_{w}—CH₂CH₂NH; and w is 4. In some embodiments, Z is —C(═O)NH—; L¹ is -L²-; L² is —(CH₂CH₂O)_{w}—CH₂CH₂NH; and w is 6. In some embodiments, Z is —NHC(═O)—; L¹ is -L²-; L² is —(CH₂CH₂O)—CH₂CH₂NH; and w is 2. In some embodiments, Z is —NHC(═O)—; L¹ is -L²-; L² is —(CH₂CH₂O)_{w}—CH₂CH₂NH; and w is 4. In some embodiments, Z is —NHC(═O)—; L¹ is -L²-; L² is —(CH₂CH₂O)_{w}—CH₂CH₂NH; and w is 6. In some embodiments, Z is —NHC(═O)NH—; L¹ is -L²-; L² is —(CH₂CH₂O)_{w}—CH₂CH₂NH; and w is 2. In some embodiments, Z is —NHC(═O)NH—; L¹ is -L²-; L² is —(CH₂CH₂O)_{w}—CH₂CH₂NH; and w is 4. In some embodiments, Z is —NHC(—O)NH—; L¹ is -L²-; L² is —(CH₂CH₂O)_{w}—CH₂CH₂NH; and w is 6.

In some embodiments, Z is —C(—O)NH—; L¹ is -L²-L⁷-; L² is —(CH₂CH₂O)_{w}—CH₂CH₂—; and w is 2. In some embodiments, Z is —C(═O)NH—; L¹ is -L²-L⁷-; L² is —(CH₂CH₂O)_{w}—CH₂CH₂—; and w is 4. In some embodiments, Z is —C(═O)NH—; L¹ is -L²-L⁷-; L² is —(CH₂CH₂O)_{w}—CH₂CH₂—; and w is 6. In some embodiments, Z is —NHC(═O)—; L¹ is -L²-L⁷-; L² is —(CH₂CH₂O)_{w}—CH₂CH₂—; and w is 2. In some embodiments, Z is —NHC(═O)—; L¹ is -L²-L⁷-; L² is —(CH₂CH₂O)_{w}—CH₂CH₂—; and w is 4. In some embodiments, Z is —NHC(═O)—; L¹ is -L²-L⁷-; L² is —(CH₂CH₂O)_{w}—CH₂CH₂—; and w is 6. In some embodiments, Z is —NHC(═O)NH—; L¹ is -L²-L⁷-; L² is —(CH₂CH₂O)_{w}—CH₂CH₂—; and w is 2. In some embodiments, Z is —NHC(—O)NH—; L¹ is -L²-L⁷; L² is —(CH₂CH₂O)_{w}—CH₂CH₂—; and w is 4. In some embodiments, Z is —NHC(═O)NH—; L¹ is -L²-L⁷; L² is —(CH₂CH₂O)_{w}—CH₂CH₂—; and w is 6.

In some embodiments, Z is —NHC(═O)—; L¹ is -L²-L⁷-; L² is substituted or unsubstituted C₁-C₂₀alkylene-C(═O)NH—; L⁷ is N-heterocycloalkylene.

In some embodiments, Z is —NHC(═O)NH—; L¹ is -L²-L⁴-L⁷-; L² is substituted or unsubstituted C₁-C₂₀alkylene-NHC(═O)—; L⁴ is C₁-C₆ alkylene that is substituted with 2-OH; and L¹ is —NH—.

In some embodiments, Z is —NHC(═O)—; L¹ is -L⁶-; L¹ is -L⁸-L⁹-L¹⁰-; L⁸ is —(CH₂)ₜ—C(═O)NR^{w}—; R^{w is —(CH}₂CH₂O)ₛ—CH₂CH₂CO₂H; s is 2; L¹ is substituted or unsubstituted heterocycloalkylene; and L¹⁰ is absent.

In some embodiments, Z is —NHC(═O)NH—; L¹ is -L²-L⁴-L⁷; L² is substituted or unsubstituted C₁-C₂₀alkylene-NHC(═O)—; L⁴ is C₁-C₆ alkylene that is substituted with 1-NH₂; and L⁷ is —NH—.

In some embodiments, Z is —NHC(═O)—; L¹ is -L²-L⁴-L⁷-; L² is —(CH₂CH₂O)_{w}—CH₂CH₂NHC(═O)—; w is 2; L⁶ is -L⁸-L⁹-L¹⁰; L⁸ is absent; L¹ is substituted or unsubstituted heterocycloalkylene; L¹⁰ is(CH₂) r; r is 1; and L⁷ is —NH—.

In some embodiments, Z is —NHC(═O)—; L¹ is -L³-L⁴-L⁷-; L³ is a peptide wherein the N atom of the amide linking the amino acids is substituted with —CH₃; L⁴ is —C(═O)CH₂CH₂—; and L¹ is —NH—. In some embodiments, L³ is penta-sarcosine.

In some embodiments, Z is —NHC(—O)—; L¹ is -L²-L³-; L² is —(CH₂CH₂O)_{w}—CH₂CH₂NH—; w is 4; and L³ is an amino acid. In some embodiments, L³ is L-cysteic acid.

In some embodiments, L¹ is: —CH₂—, —CH₂CH₂—, —CH₂CH₂CH₂—, —CH₂CH₂CH₂CH₂—,

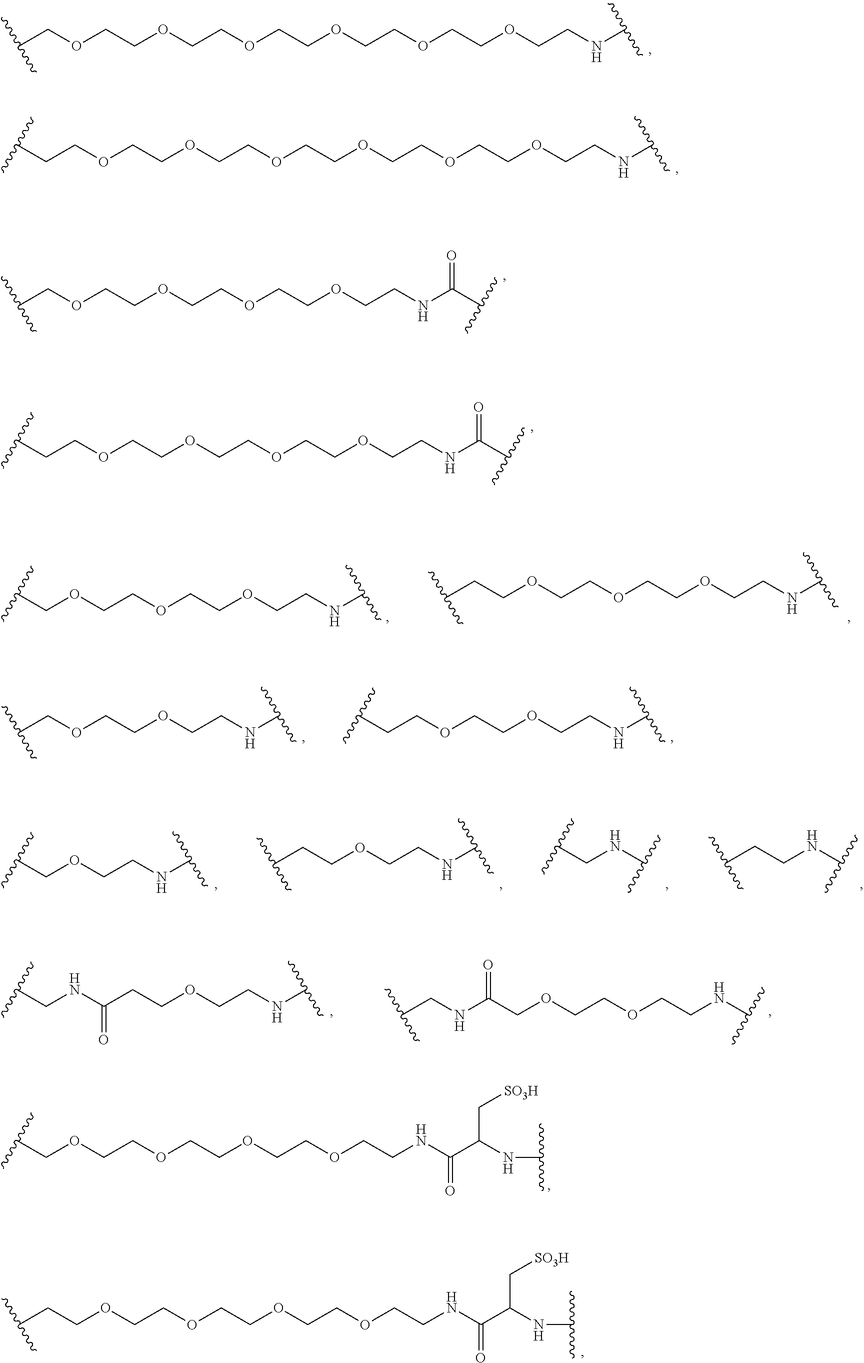

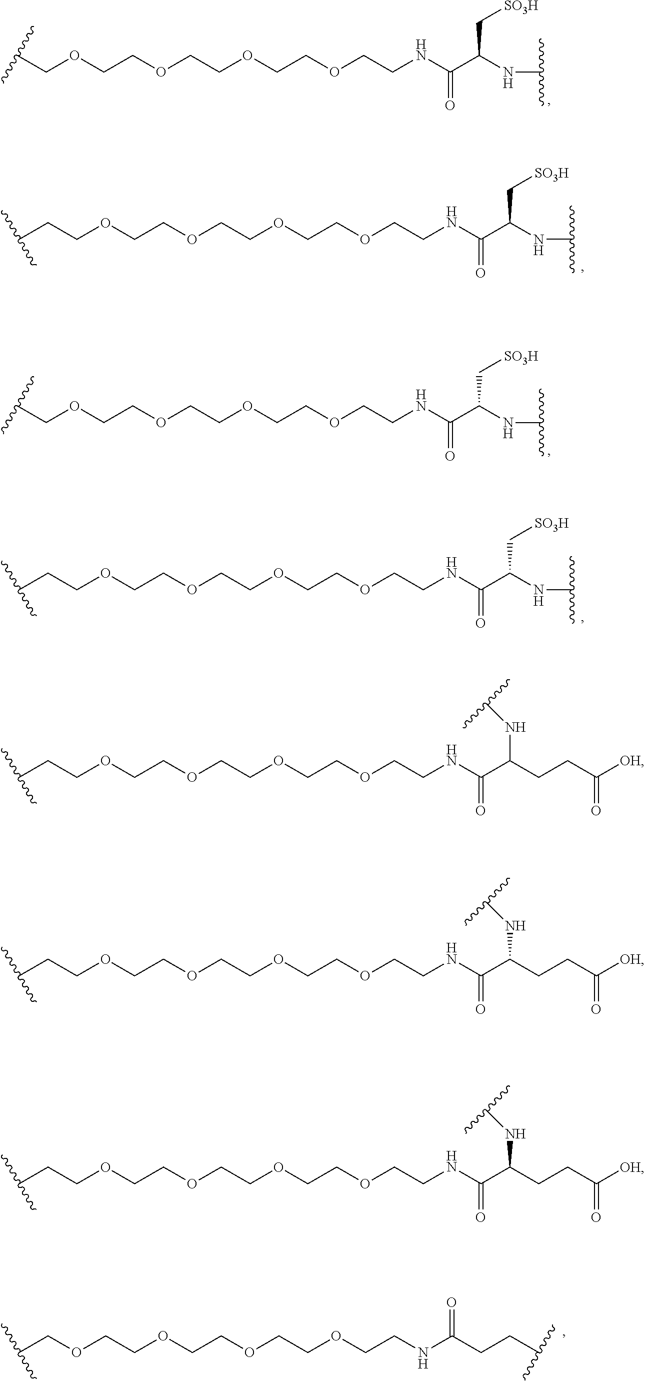

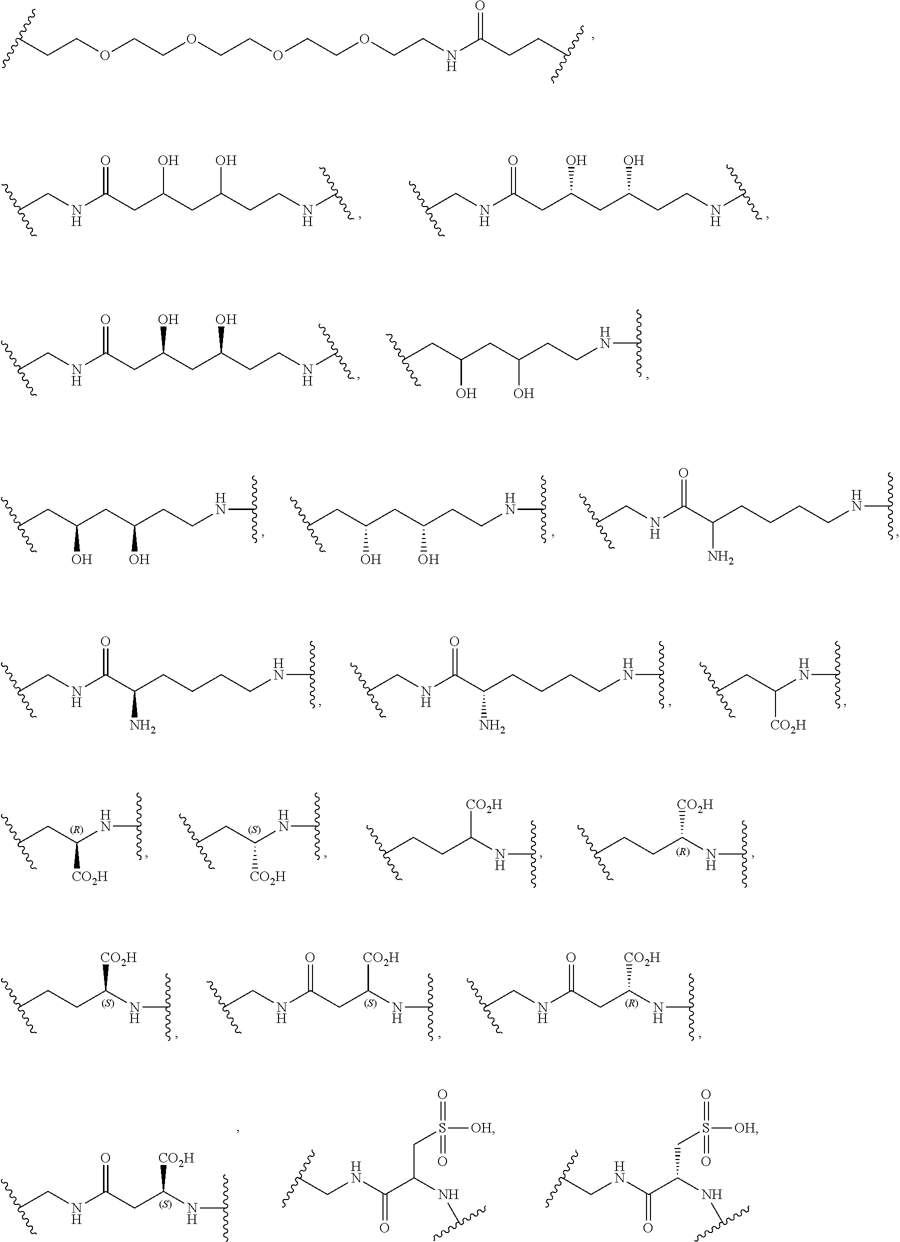

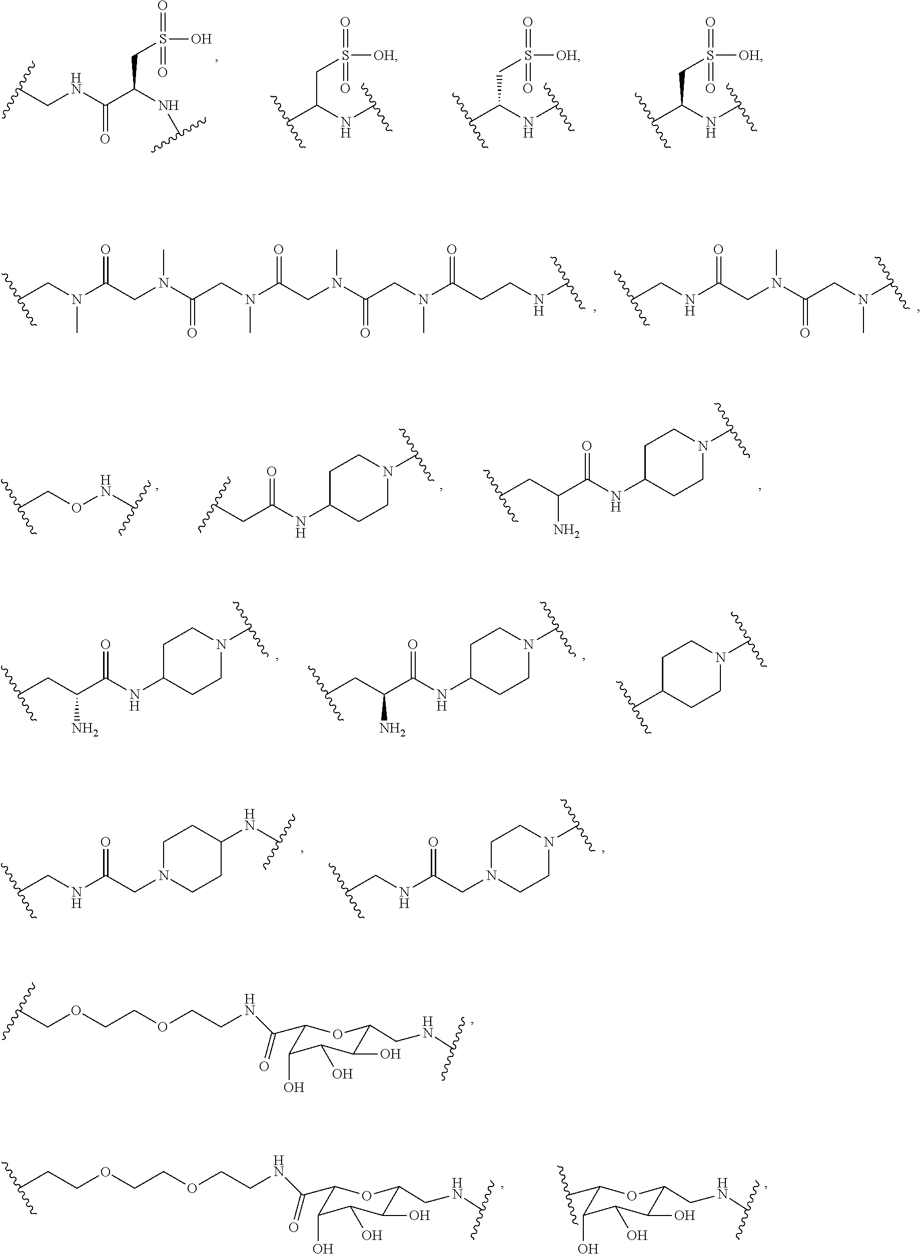

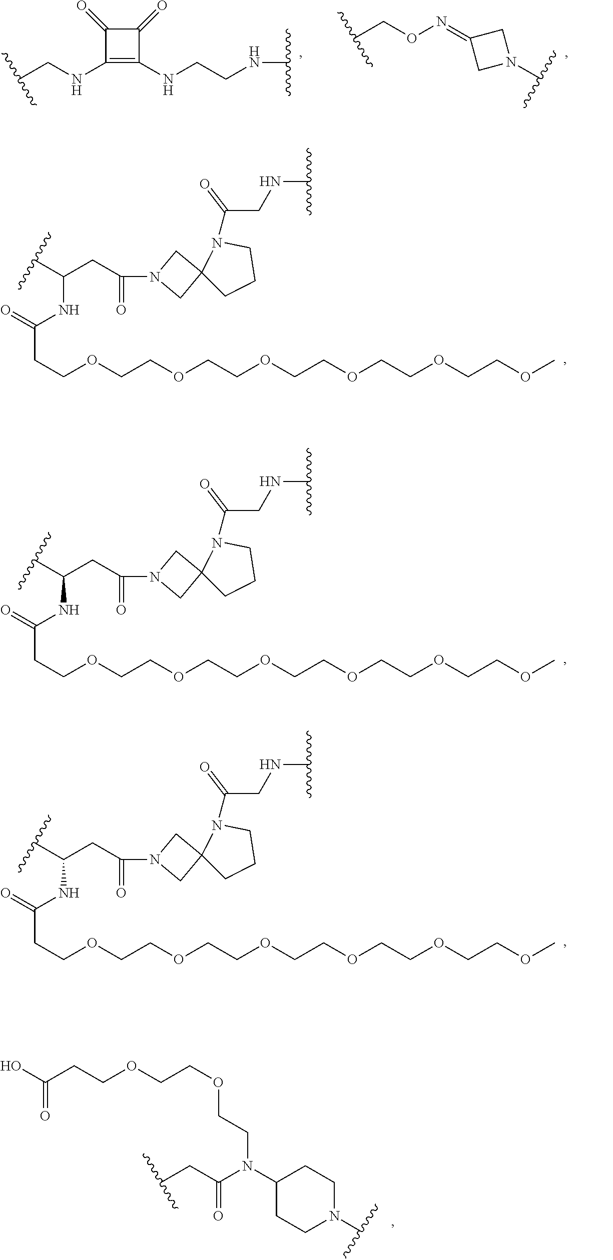 [SMILES: *CNc1c(NCCN*)c(=O)c1=O ; *CON=C1CN(*)C1 ; *NCC(=O)N1CCCC12CN(C(=O)CC(*)NC(=O)CCOCCOCCOCCOCCOCCOC)C2 ; *NCC(=O)N1CCCC12CN(C(=O)C[C@H](*)NC(=O)CCOCCOCCOCCOCCOCCOC)C2 ; *NCC(=O)N1CCCC12CN(C(=O)C[C@@H](*)NC(=O)CCOCCOCCOCCOCCOCCOC)C2 ; *CC(=O)N(CCOCCOCCC(=O)O)C1CCN(*)CC1]

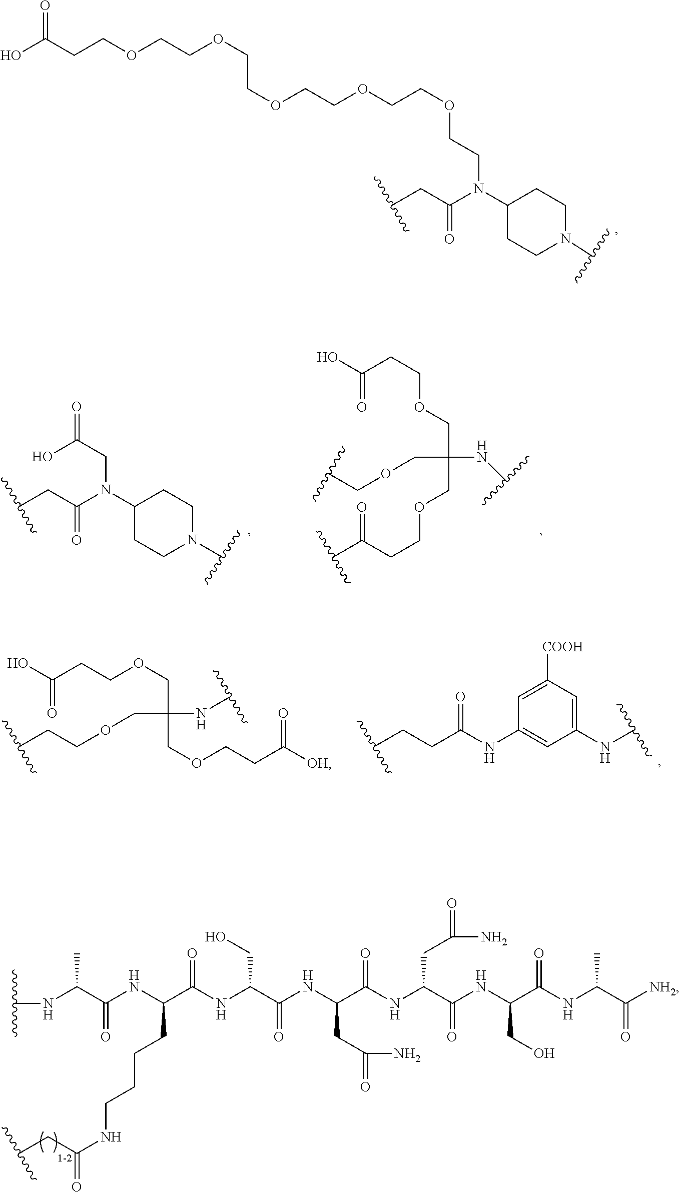

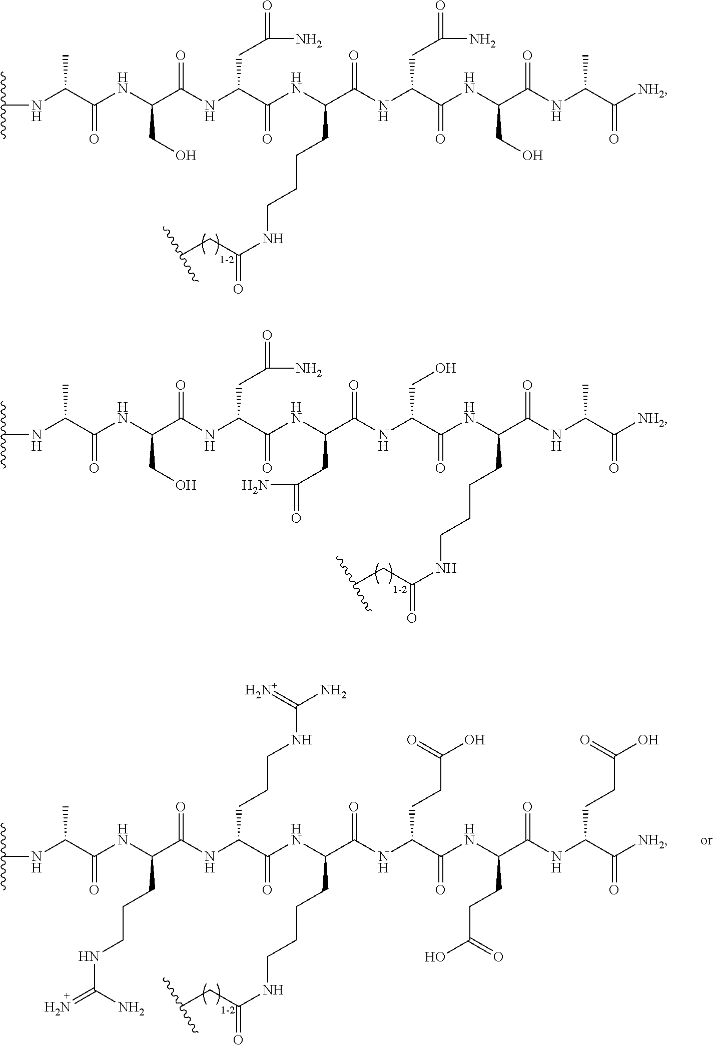

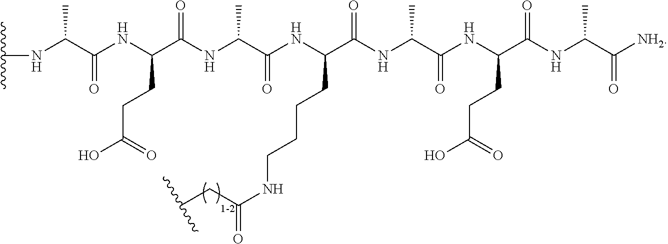

In some embodiments, L¹ is:

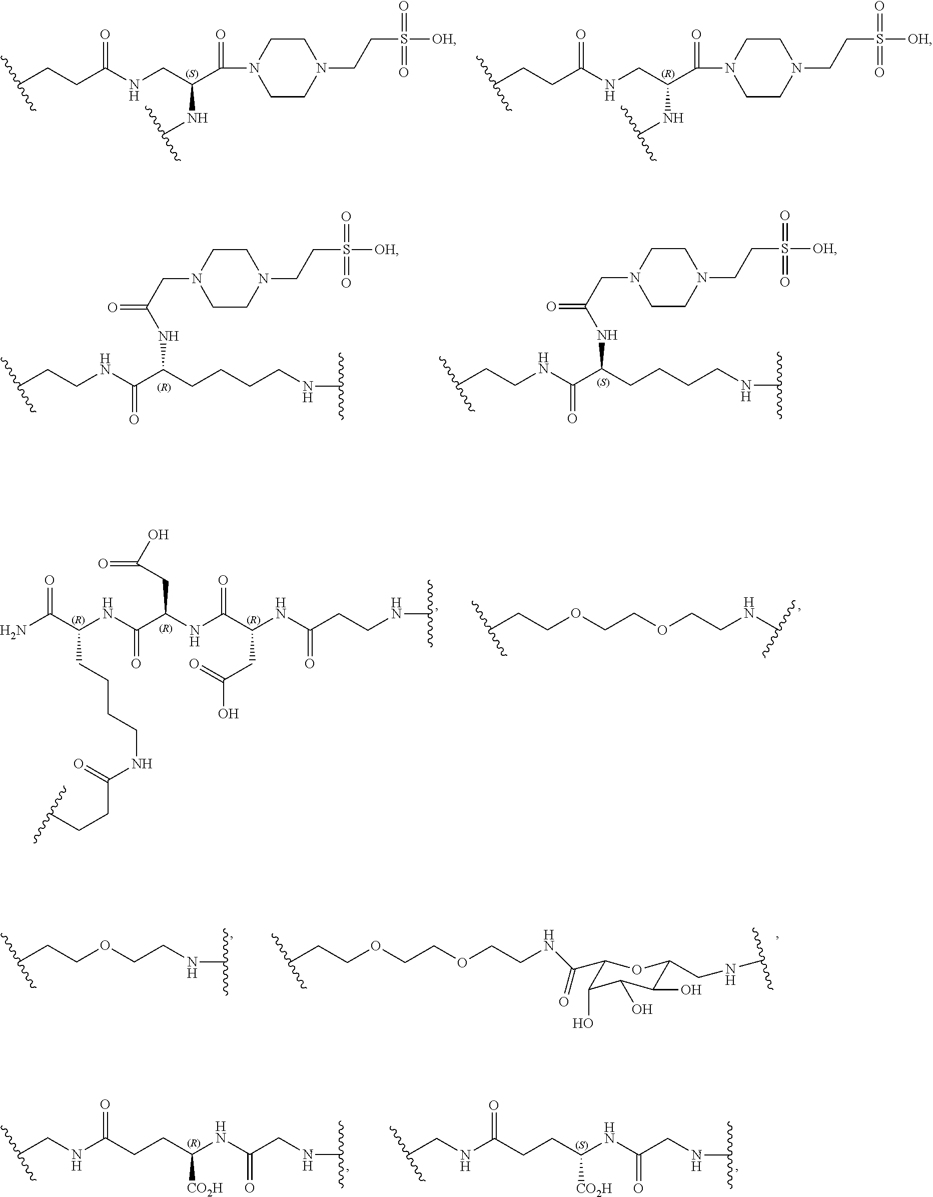

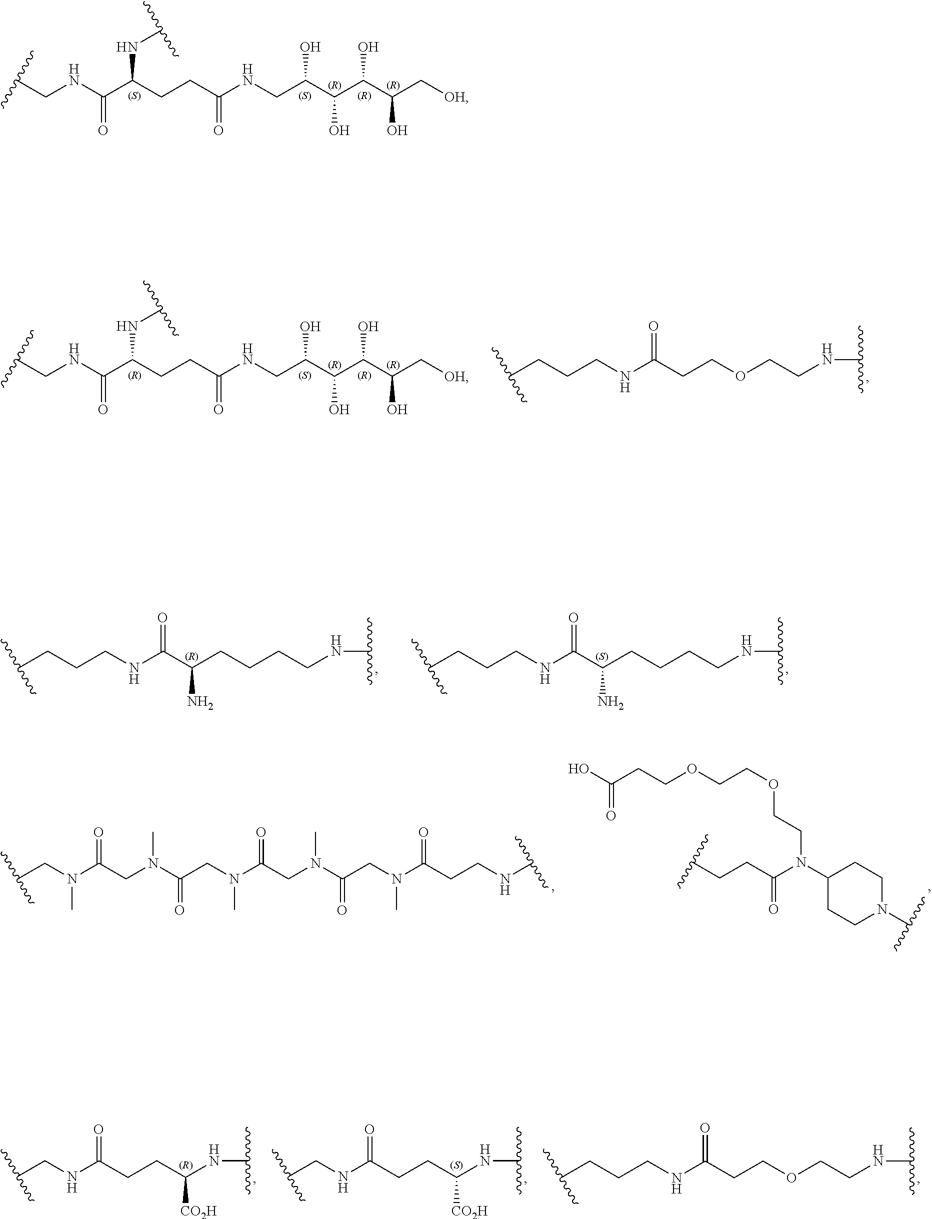

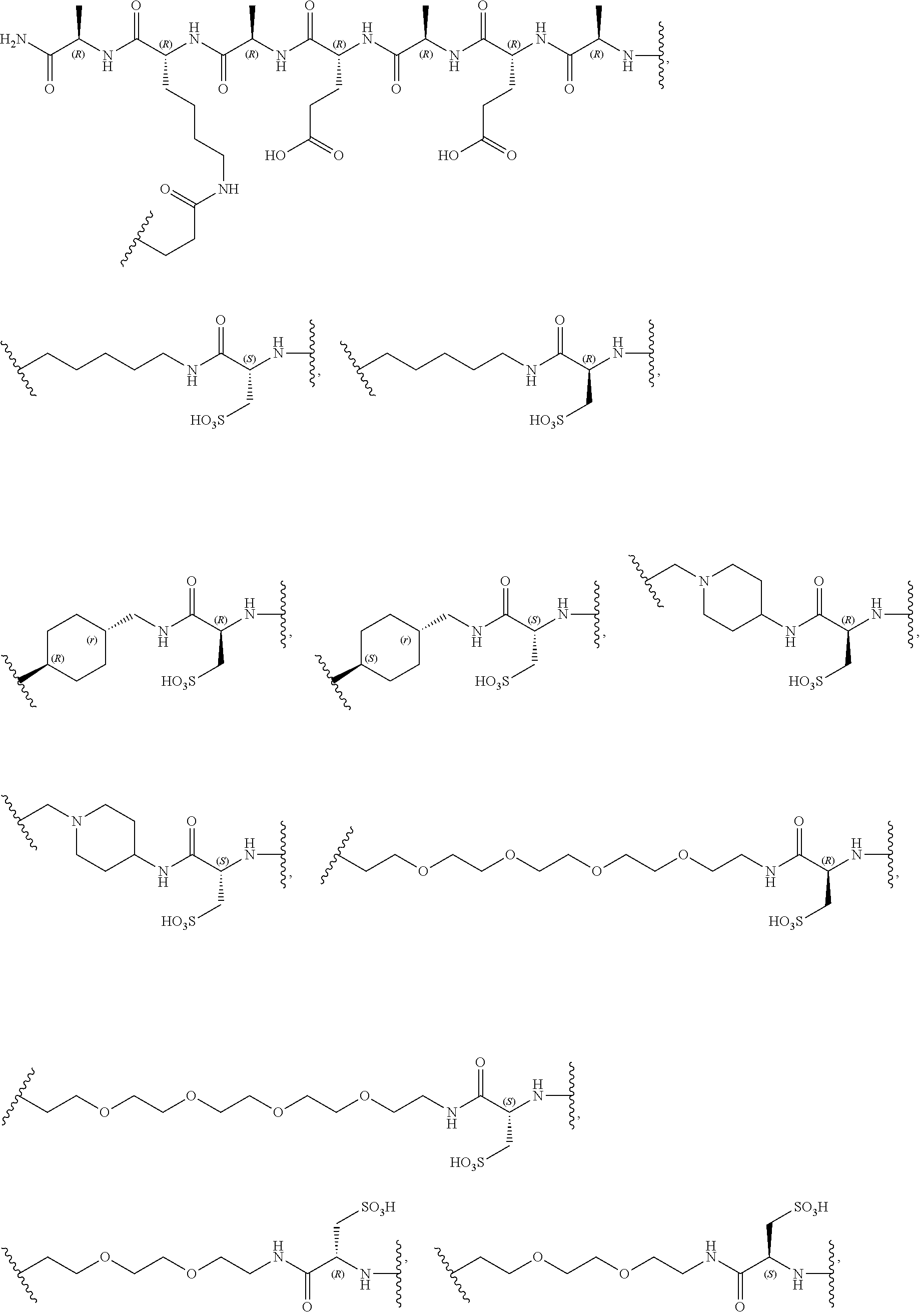

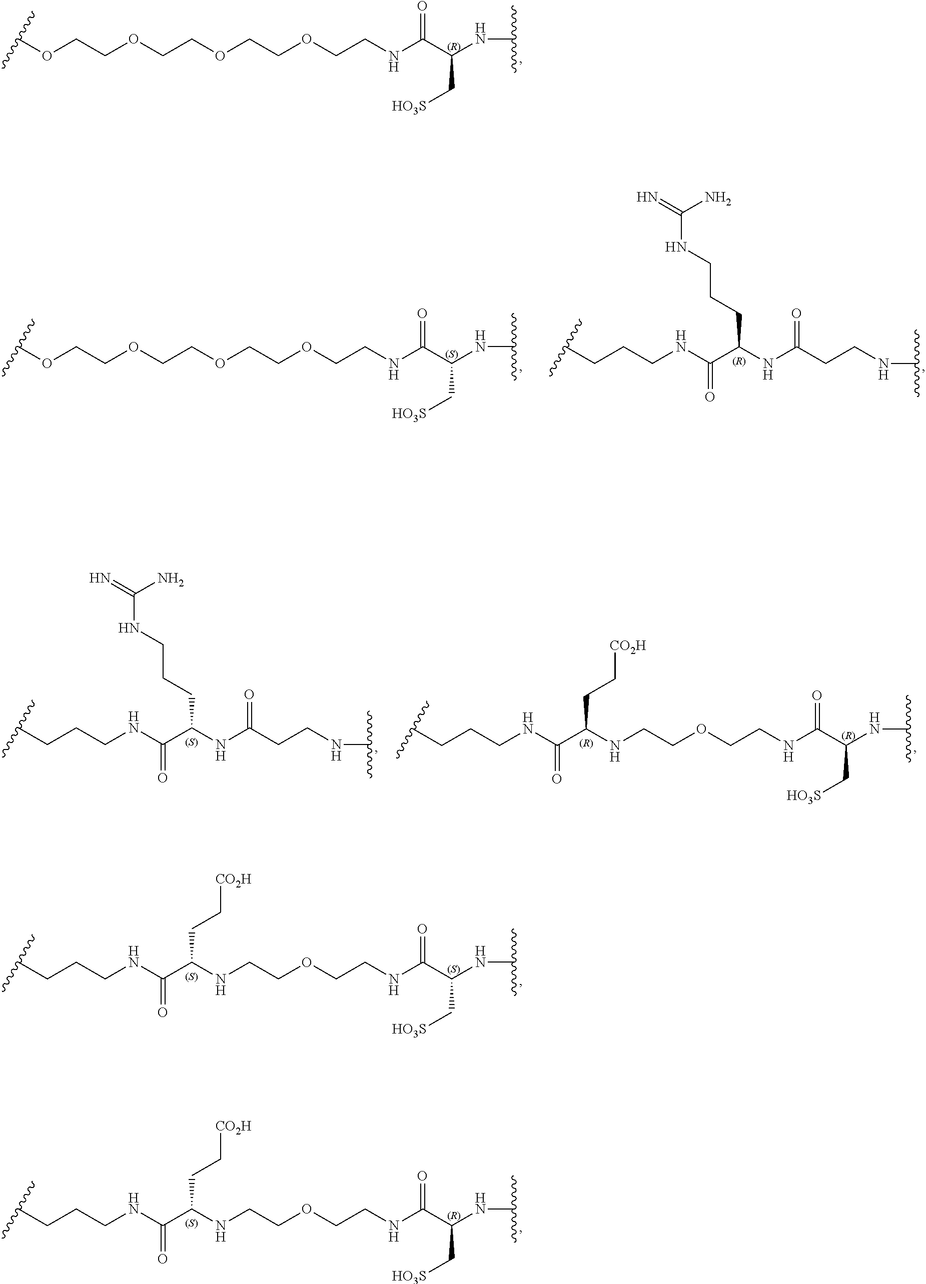

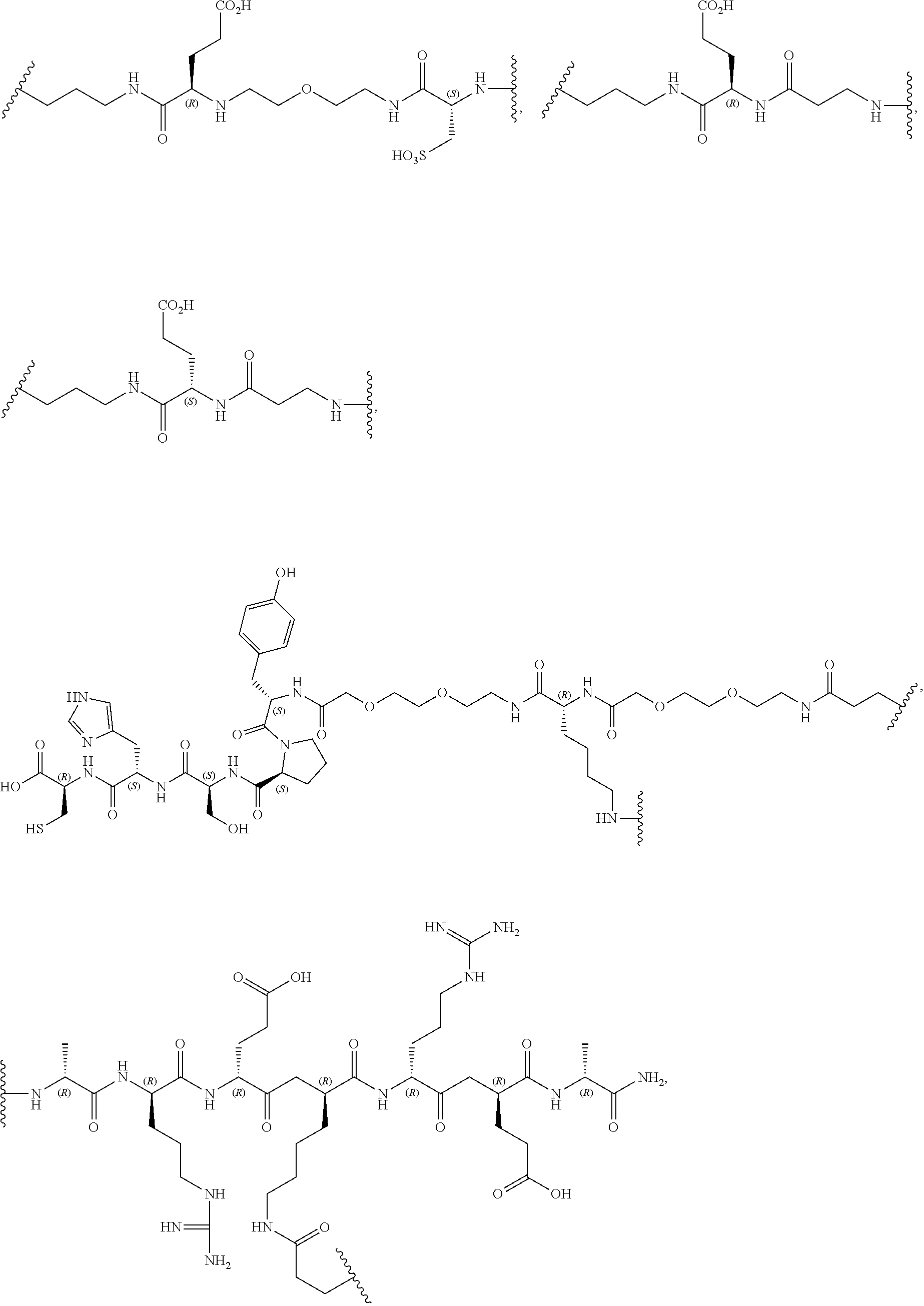

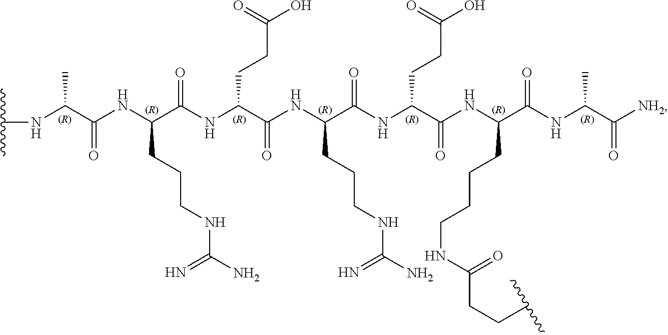

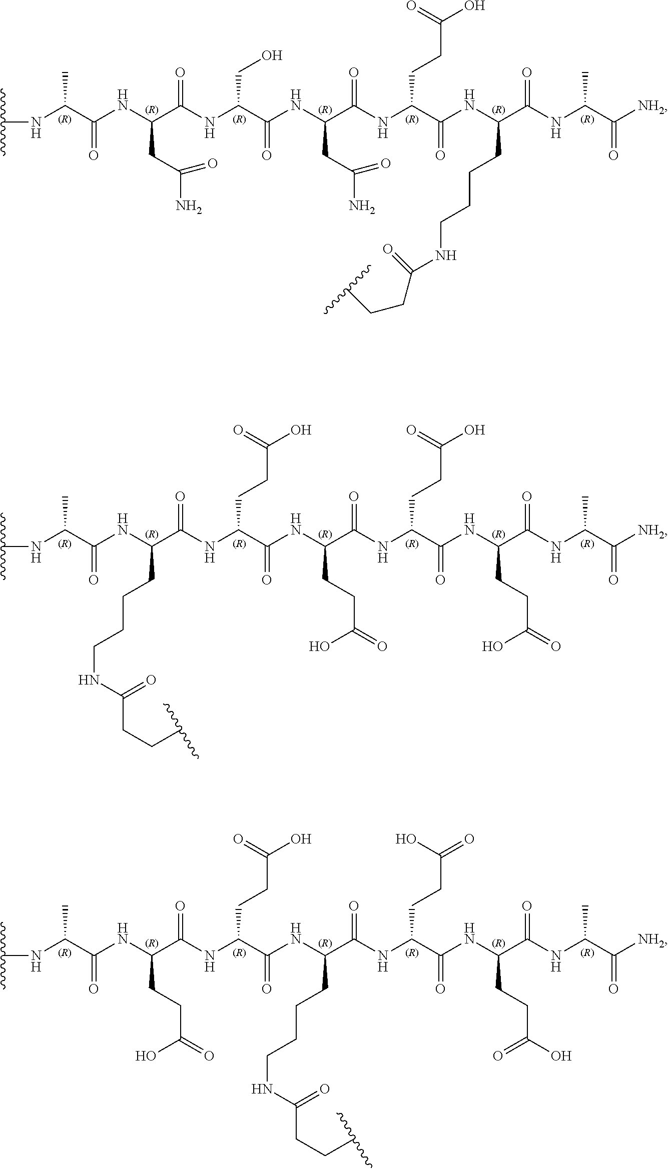

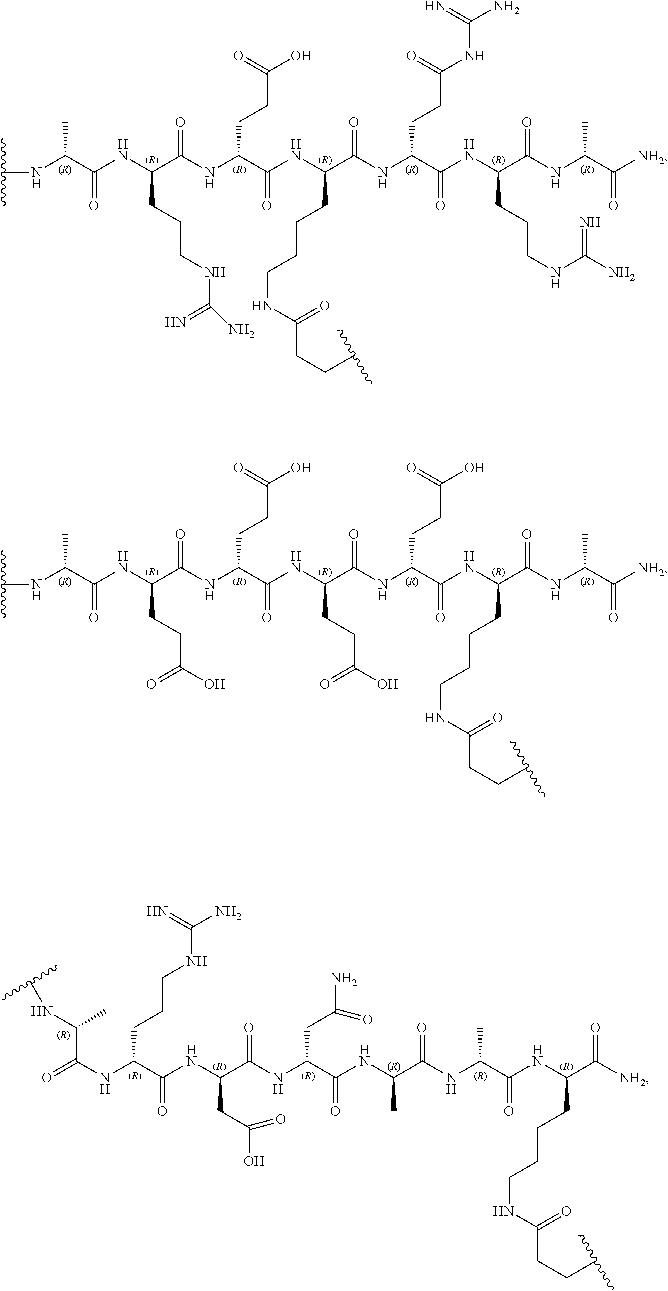

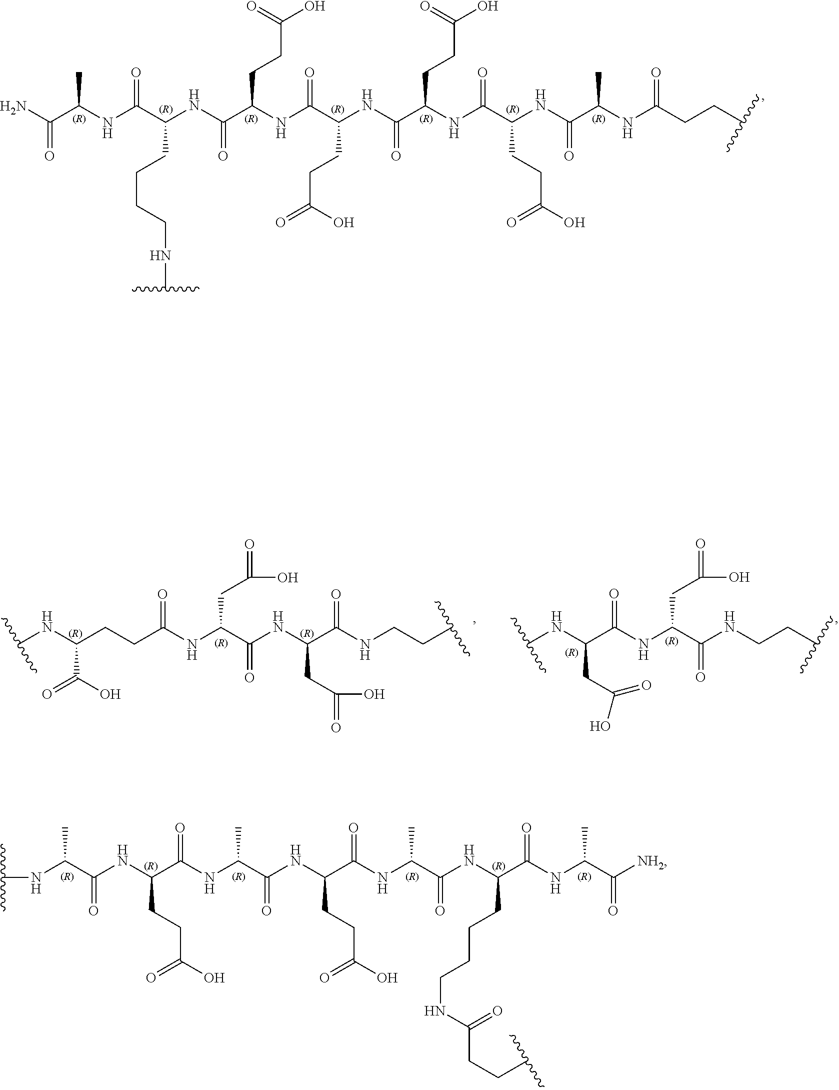

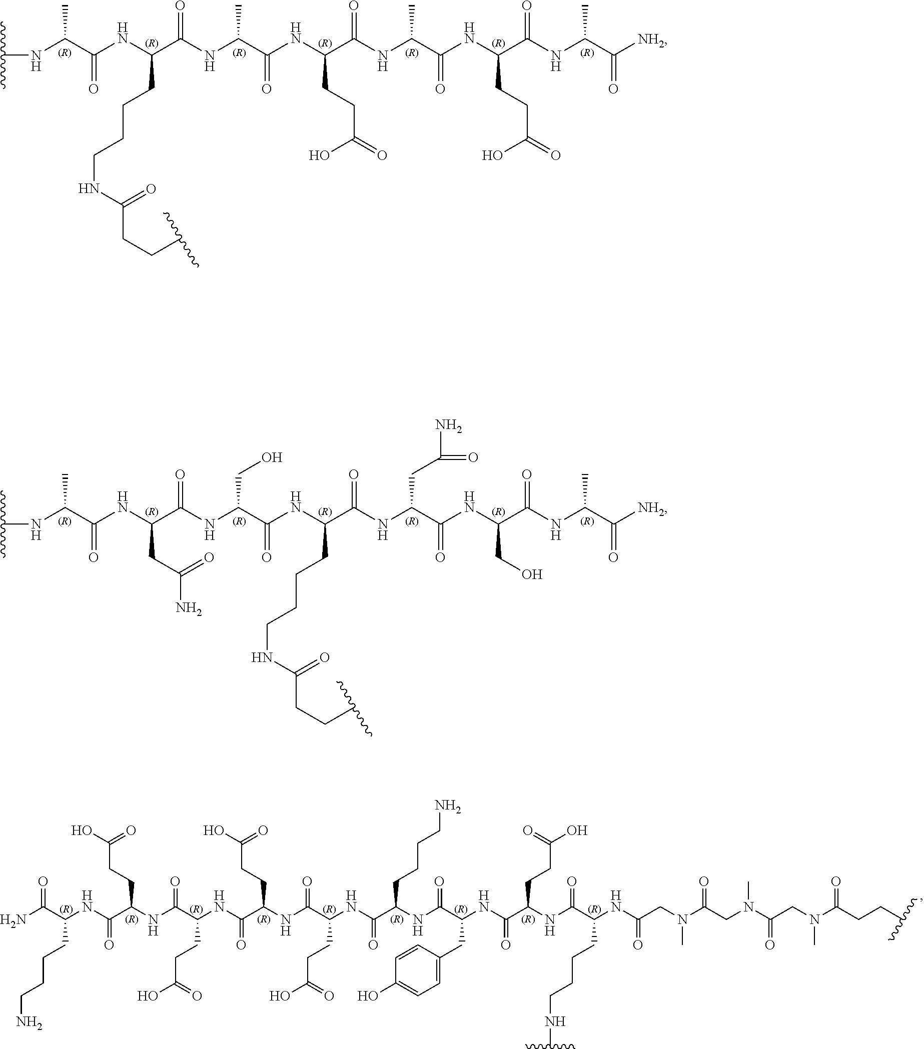

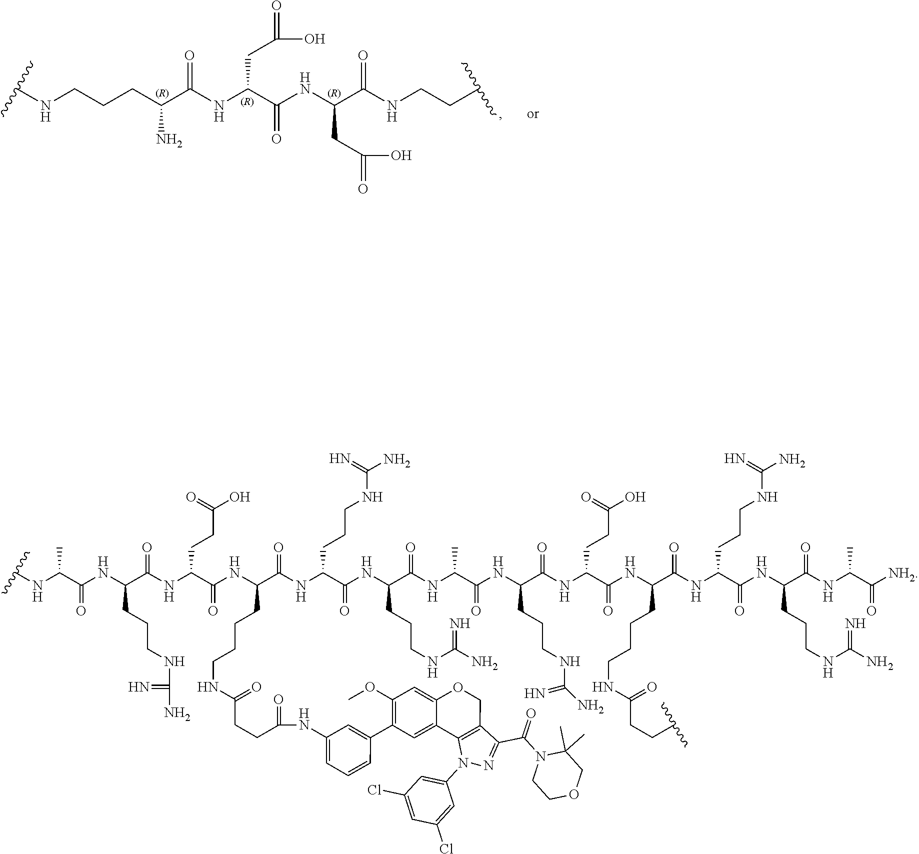

In some embodiments, L¹ is: —CH₂—. In some embodiments, L¹ is:

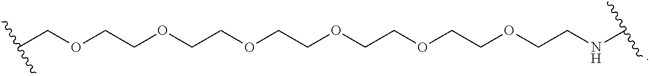 [SMILES: *COCCOCCOCCOCCOCCOCCN* ; "."]

In some embodiments, L¹ is:

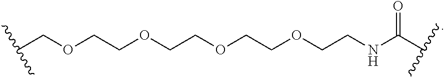 [SMILES: *COCCOCCOCCOCCNC(*)=O]

In some embodiments, L¹ is:

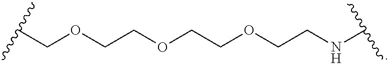 [SMILES: *COCCOCCOCCN*]

In some embodiments, L¹ is:

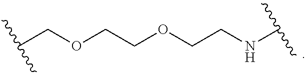 [SMILES: *COCCOCCN*]

In some embodiments, L¹ is:

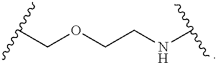 [SMILES: *COCCN*]

In some embodiments, L¹ is:

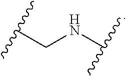 [SMILES: *CN*]

In some embodiments, L¹ is:

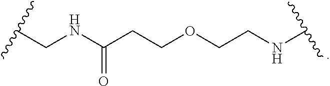

In some embodiments, L¹ is:

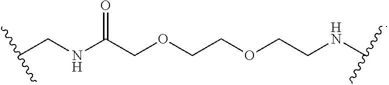 [SMILES: *CNC(=O)COCCOCCN*]

In some embodiments, L¹ is:

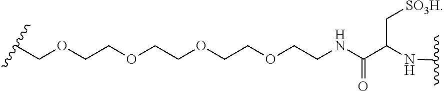 [SMILES: *COCCOCCOCCOCCNC(=O)C(CS(=O)(=O)O)N*]

In some embodiments, L¹ is:

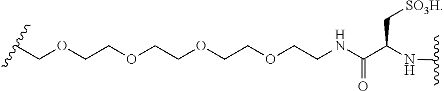 [SMILES: *COCCOCCOCCOCCNC(=O)[C@@H](CS(=O)(=O)O)N*]

In some embodiments, L¹ is:

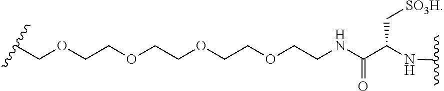 [SMILES: *COCCOCCOCCOCCNC(=O)[C@H](CS(=O)(=O)O)N*]

In some embodiments, L¹ is:

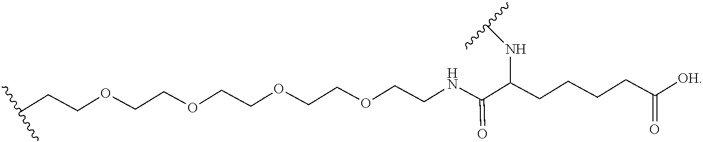 [SMILES: *CCOCCOCCOCCOCCNC(=O)C(CCCCC(=O)O)N*]

In some embodiments, L¹ is:

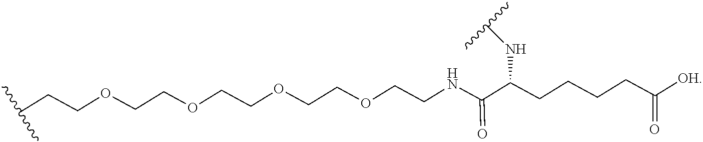 [SMILES: *CCOCCOCCOCCOCCNC(=O)[C@@H](CCCCC(=O)O)N*]

In some embodiments, L¹ is:

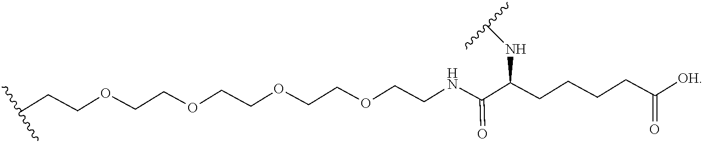 [SMILES: *CCOCCOCCOCCOCCNC(=O)[C@H](CCCCC(=O)O)N*]

In some embodiments, L¹ is:

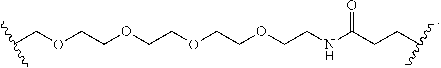 [SMILES: *CCC(=O)NCCOCCOCCOCCOC*]

In some embodiments, L¹ is:

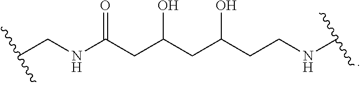 [SMILES: *CNC(=O)CC(O)CC(O)CCN*]

In some embodiments, L¹ is:

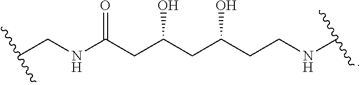 [SMILES: *CNC(=O)C[C@H](O)C[C@H](O)CCN*]

In some embodiments, L¹ is:

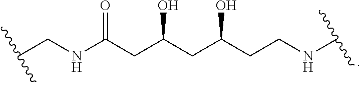 [SMILES: *CNC(=O)C[C@@H](O)C[C@@H](O)CCN*]

In some embodiments, L¹ is:

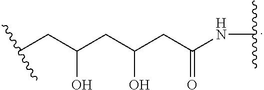 [SMILES: *CC(O)CC(O)CC(=O)N*]

In some embodiments, L¹ is:

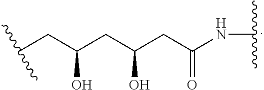 [SMILES: *C[C@H](O)C[C@H](O)CC(=O)N*]

In some embodiments, L¹ is:

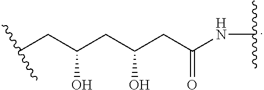 [SMILES: *C[C@@H](O)C[C@@H](O)CC(=O)N*]

In some embodiments, L¹ is:

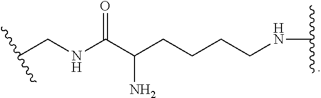 [SMILES: *CNC(=O)C(N)CCCCN*]

In some embodiments, L¹ is:

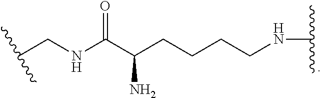 [SMILES: *CNC(=O)[C@H](N)CCCCN*]

In some embodiments, L¹ is:

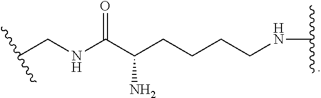 [SMILES: *CNC(=O)[C@@H](N)CCCCN*]

In some embodiments, L¹ is:

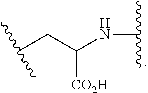 [SMILES: *CC(N*)C(=O)O]

In some embodiments, L¹ is:

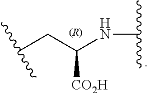

In some embodiments, L¹ is:

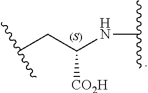

In some embodiments, L¹ is:

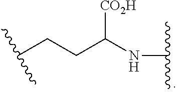 [SMILES: *CCC(N*)C(=O)O]

In some embodiments, L¹ is:

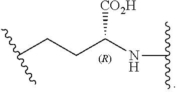

In some embodiments, L¹ is:

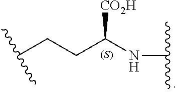

In some embodiments, L¹ is:

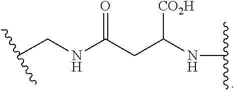 [SMILES: *CNC(=O)CC(N*)C(=O)O]

In some embodiments, L¹ is:

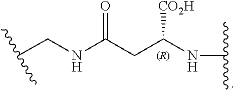

In some embodiments, L¹ is:

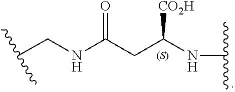

In some embodiments, L¹ is:

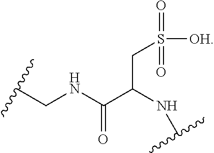 [SMILES: *CNC(=O)C(CS(=O)(=O)O)N*]

In some embodiments, L¹ is:

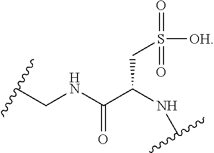 [SMILES: *CNC(=O)[C@H](CS(=O)(=O)O)N*]

In some embodiments, L¹ is:

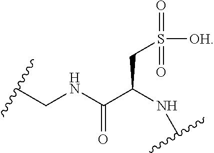 [SMILES: *CNC(=O)[C@@H](CS(=O)(=O)O)N*]

In some embodiments, L¹ is:

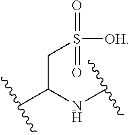 [SMILES: *NC(*)CS(=O)(=O)O]

In some embodiments, L¹ is:

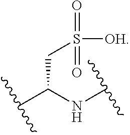 [SMILES: *N[C@H](*)CS(=O)(=O)O]

In some embodiments, L¹ is:

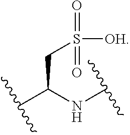 [SMILES: *N[C@@H](*)CS(=O)(=O)O]

In some embodiments, L¹ is:

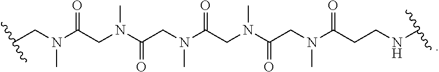 [SMILES: *CN(C)C(=O)CN(C)C(=O)CN(C)C(=O)CN(C)C(=O)CN(C)C(=O)CCN*]

In some embodiments, L¹ is:

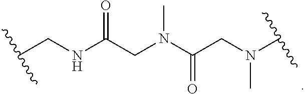 [SMILES: *CNC(=O)CN(C)C(=O)CN(*)C]

In some embodiments, L¹ is:

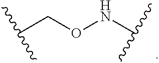 [SMILES: *CON*]

In some embodiments, L¹ is:

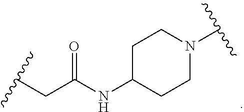 [SMILES: *CC(=O)NC1CCN(*)CC1]

In some embodiments, L¹ is:

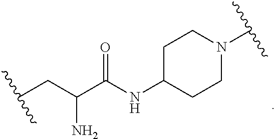 [SMILES: *CC(N)C(=O)NC1CCN(*)CC1]

In some embodiments, L¹ is:

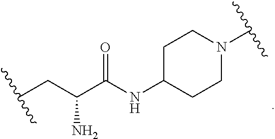 [SMILES: *C[C@@H](N)C(=O)NC1CCN(*)CC1]

In some embodiments, L¹ is:

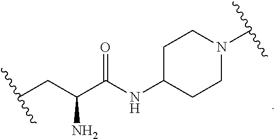 [SMILES: *C[C@H](N)C(=O)NC1CCN(*)CC1]

In some embodiments, L¹ is:

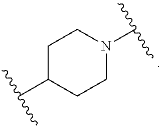 [SMILES: *C1CCN(*)CC1]

In some embodiments, L¹ is:

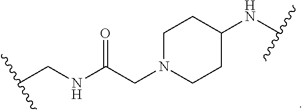 [SMILES: *CNC(=O)CN1CCC(N*)CC1]

In some embodiments, L¹ is:

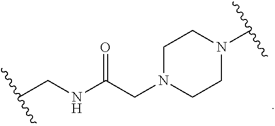 [SMILES: *CNC(=O)CN1CCN(*)CC1]

In some embodiments, L¹ is:

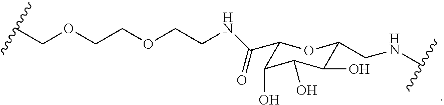 [SMILES: *COCCOCCNC(=O)C1OC(CN*)C(O)C(O)C1O]

In some embodiments, L¹ is:

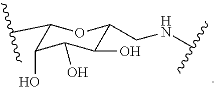 [SMILES: *NCC1OC(*)C(O)C(O)C1O]

In some embodiments, L¹ is:

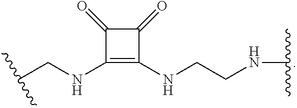 [SMILES: *CNc1c(NCCN*)c(=O)c1=O]

In some embodiments, L¹ is:

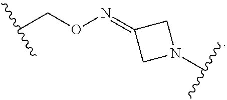 [SMILES: *CON=C1CN(*)C1]

In some embodiments, L¹ is:

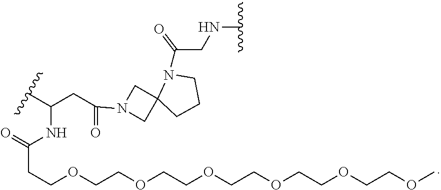 [SMILES: *NCC(=O)N1CCCC12CN(C(=O)CC(*)NC(=O)CCOCCOCCOCCOCCOCCOC)C2]

In some embodiments, L¹ is:

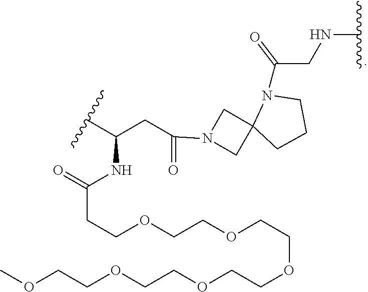 [SMILES: *NCC(=O)N1CCCC12CN(C(=O)C[C@H](*)NC(=O)CCOCCOCCOCCOCCOCCOC)C2]

In some embodiments, L¹ is:

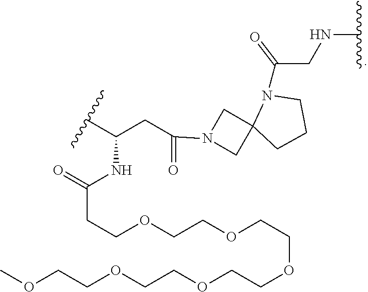 [SMILES: *NCC(=O)N1CCCC12CN(C(=O)C[C@@H](*)NC(=O)CCOCCOCCOCCOCCOCCOC)C2]

In some embodiments, L¹ is:

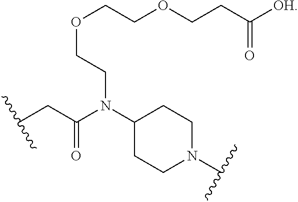 [SMILES: *CC(=O)N(CCOCCOCCC(=O)O)C1CCN(*)CC1]

In some embodiments, L¹ is:

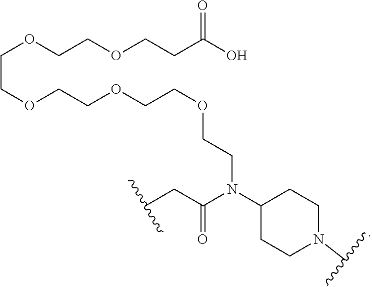 [SMILES: *CC(=O)N(CCOCCOCCOCCOCCOCCC(=O)O)C1CCN(*)CC1]

In some embodiments, L¹ is:

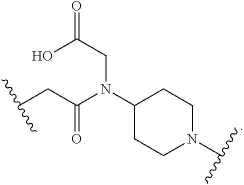 [SMILES: *CC(=O)N(CC(=O)O)C1CCN(*)CC1]

In some embodiments, L¹ is:

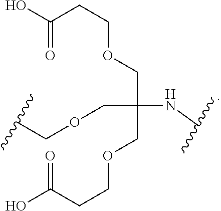 [SMILES: *COCC(COCCC(=O)O)(COCCC(=O)O)N*]

In some embodiments, L¹ is:

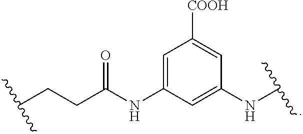 [SMILES: *CCC(=O)Nc1cc(N*)cc(C(=O)O)c1]

In some embodiments, L¹ is:

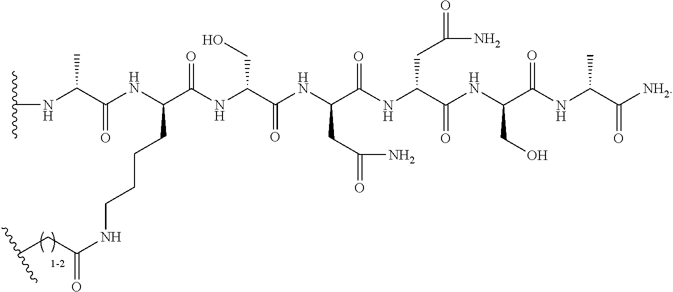

In some embodiments, L¹ is:

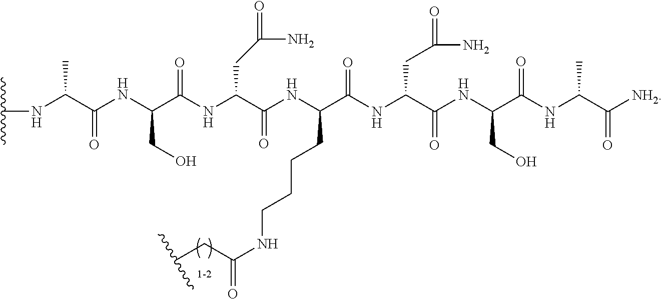

In some embodiments, L¹ is:

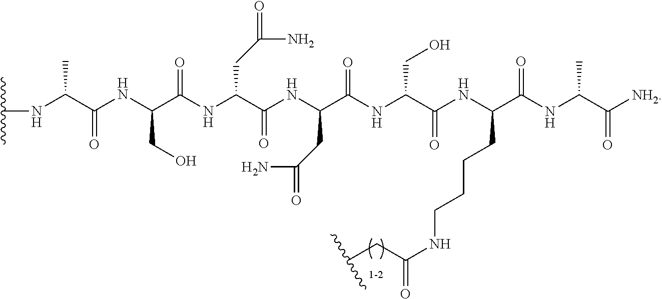

In some embodiments, L¹ is:

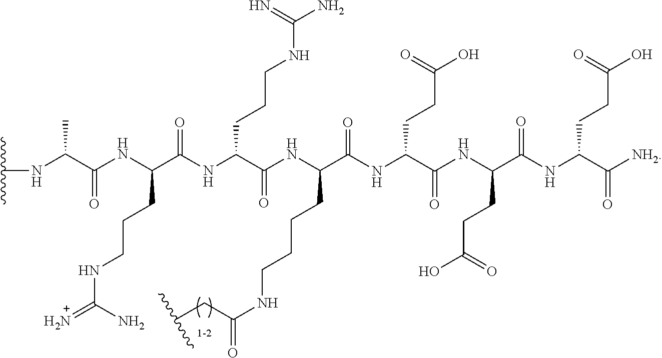

In some embodiments, L¹ is:

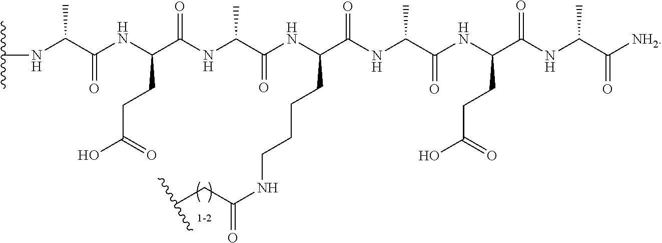

In some embodiments, L¹ is:

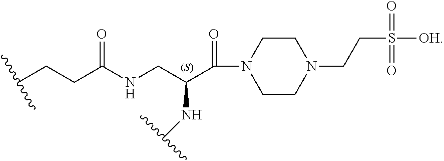

In some embodiments, L¹ is:

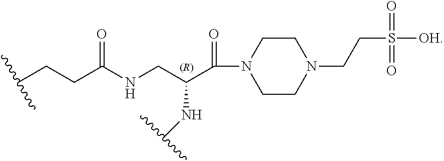

In some embodiments, L¹ is:

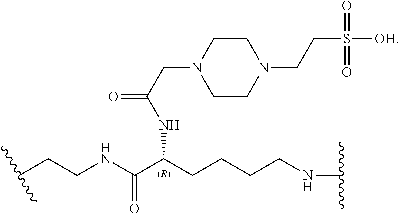

In some embodiments, L¹ is:

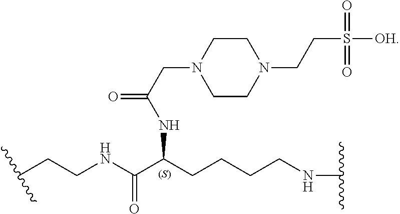

In some embodiments, L¹ is:

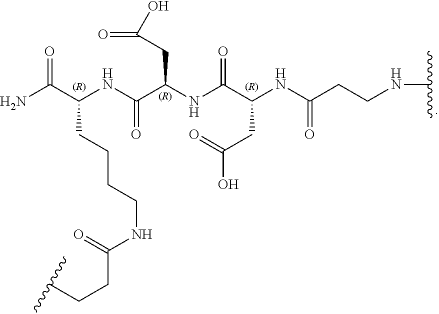

In some embodiments, L¹ is:

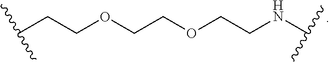 [SMILES: *CCOCCOCCN*]

In some embodiments, L¹ is:

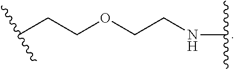 [SMILES: *CCOCCN*]

In some embodiments, L¹ is:

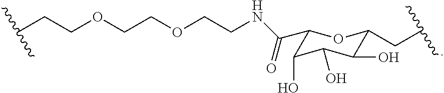 [SMILES: *CCOCCOCCNC(=O)C1OC(C*)C(O)C(O)C1O]

In some embodiments, L¹ is:

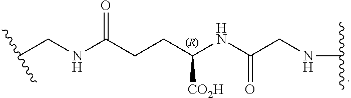

In some embodiments, L¹ is:

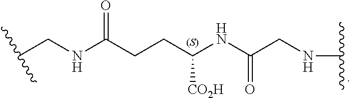

In some embodiments, L¹ is:

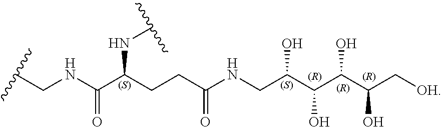

In some embodiments, L¹ is:

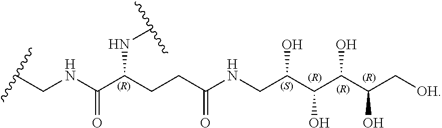

In some embodiments, L¹ is:

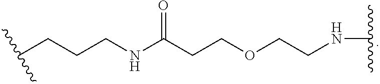 [SMILES: *CCCNC(=O)CCOCCN*]

In some embodiments, L¹ is:

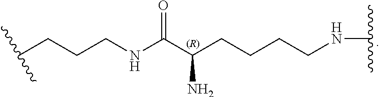

In some embodiments, L¹ is:

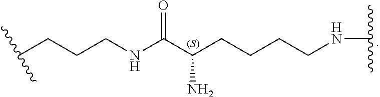

In some embodiments, L¹ is:

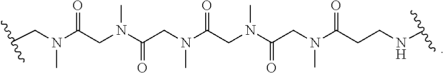 [SMILES: *CN(C)C(=O)CN(C)C(=O)CN(C)C(=O)CN(C)C(=O)CN(C)C(=O)CCN*]

In some embodiments, L¹ is:

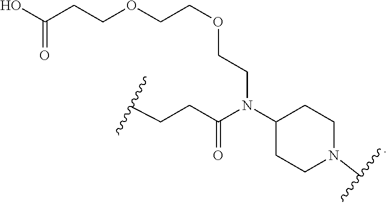 [SMILES: *CCC(=O)N(CCOCCOCCC(=O)O)C1CCN(*)CC1]

In some embodiments, L¹ is:

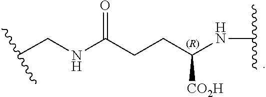

In some embodiments, L¹ is:

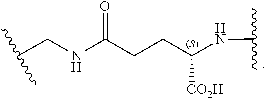

In some embodiments, L¹ is:

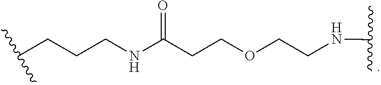 [SMILES: *CCCNC(=O)CCOCCN*]

In some embodiments, L¹ is:

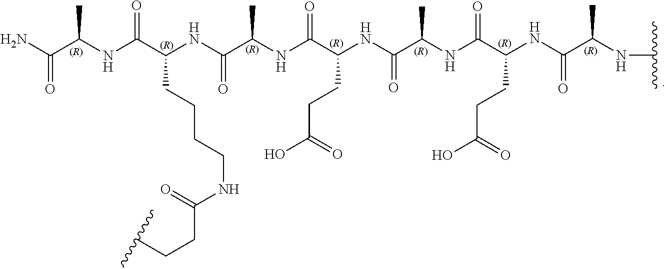

In some embodiments, L¹ is:

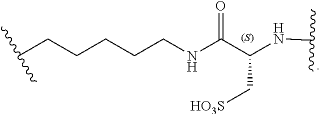

In some embodiments, L¹ is:

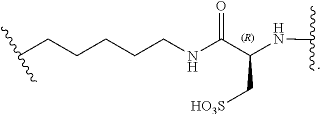

In some embodiments, L¹ is:

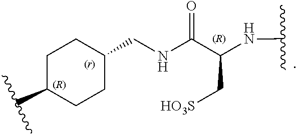

In some embodiments, L¹ is:

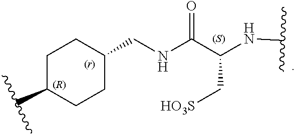

In some embodiments, L¹ is:

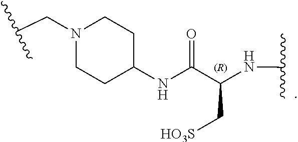

In some embodiments, L¹ is:

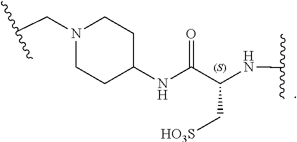

In some embodiments, L¹ is:

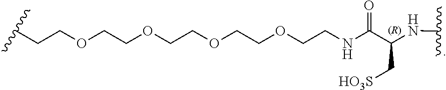

In some embodiments, L¹ is:

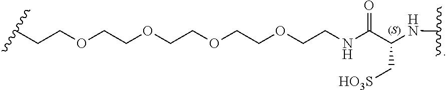

In some embodiments, L¹ is:

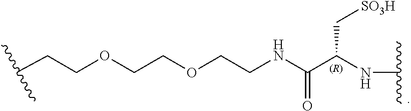

In some embodiments, L¹ is:

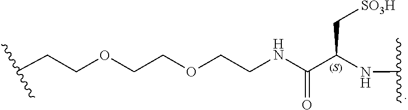

In some embodiments, L¹ is:

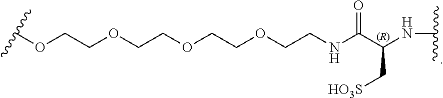

In some embodiments, L¹ is:

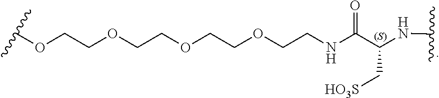

In some embodiments, L¹ is:

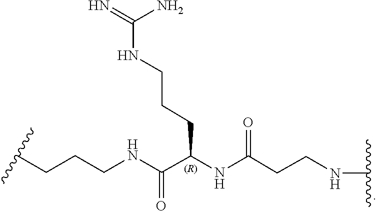

In some embodiments, L¹ is:

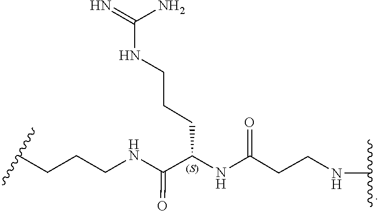

In some embodiments, L¹ is:

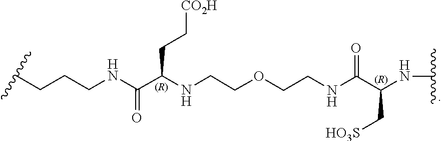

In some embodiments, L¹ is:

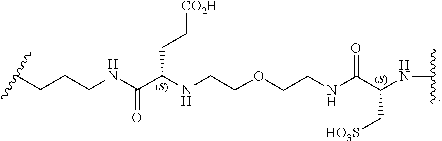

In some embodiments, L¹ is:

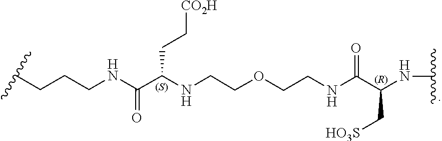

In some embodiments, L¹ is:

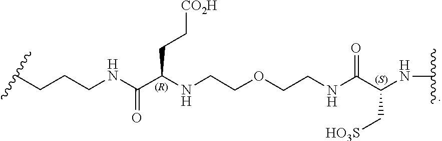

In some embodiments, L¹ is:

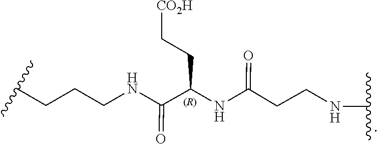

In some embodiments, L¹ is:

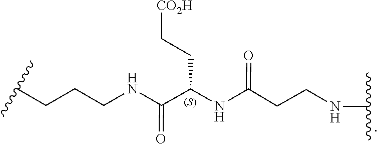

In some embodiments, L¹ is:

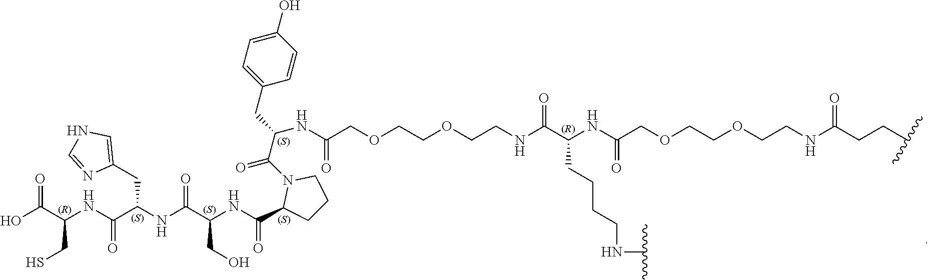

In some embodiments, L¹ is:

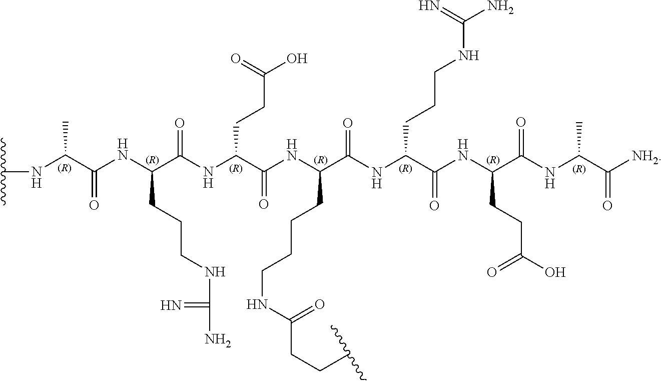

In some embodiments, L¹ is:

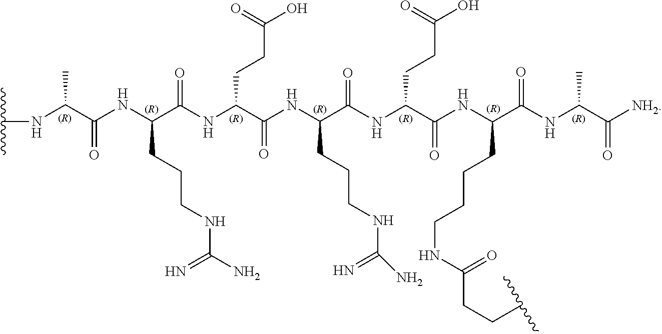

In some embodiments, L¹ is:

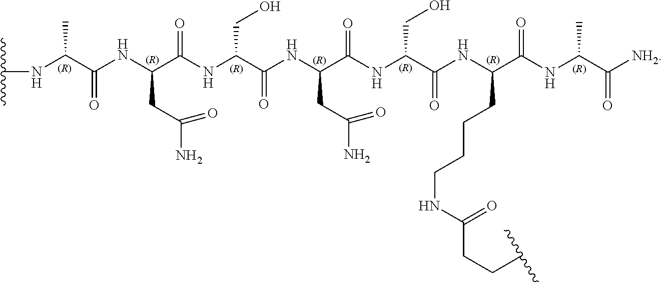

In some embodiments, L¹ is:

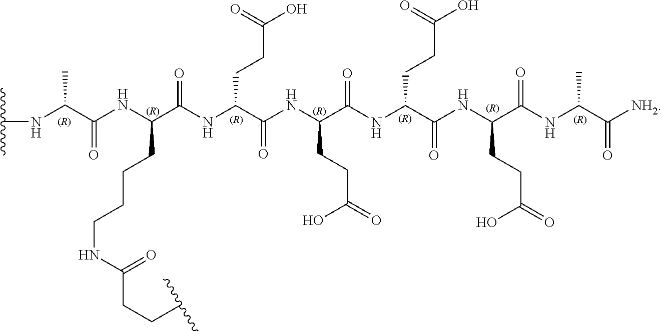

In some embodiments, L¹ is:

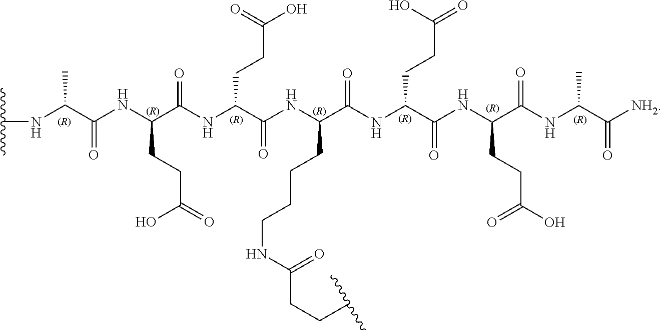

In some embodiments, L¹ is:

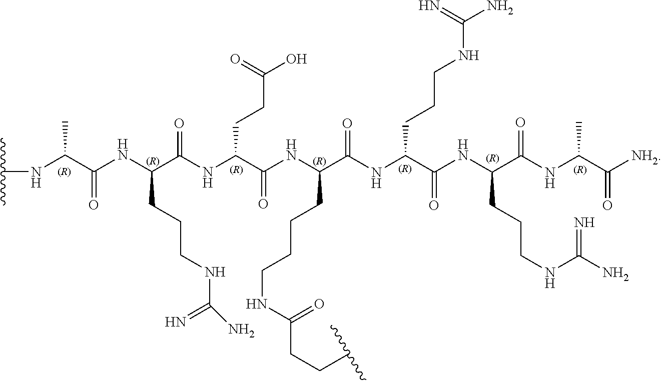

In some embodiments, L¹ is:

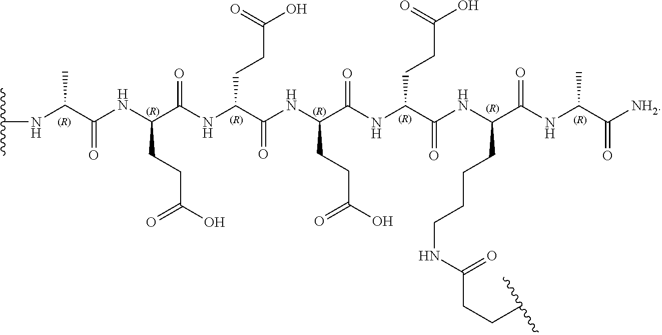

In some embodiments, L¹ is:

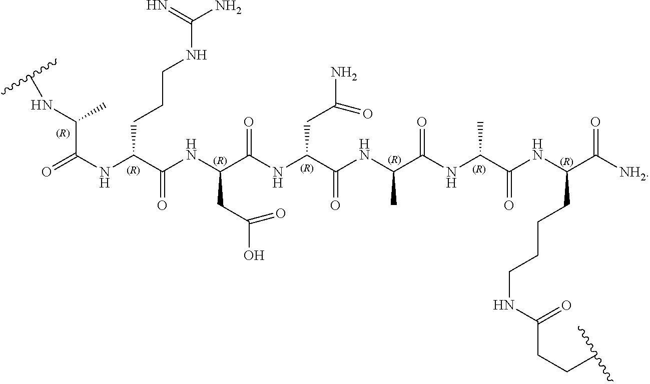

In some embodiments, L¹ is:

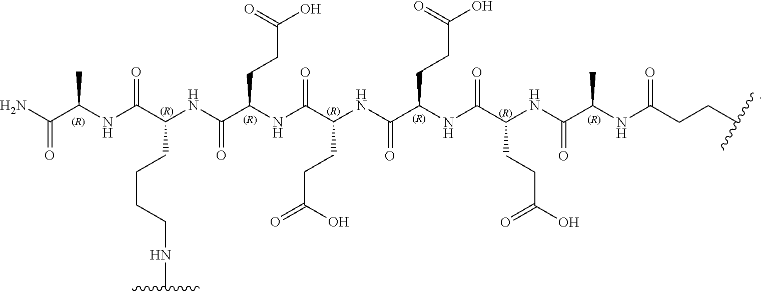

In some embodiments, L¹ is:

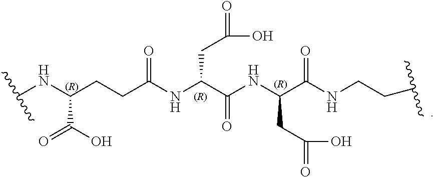

In some embodiments, L¹ is:

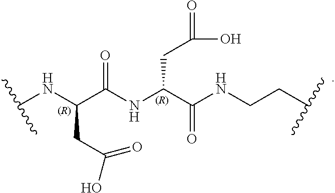

In some embodiments, L¹ is:

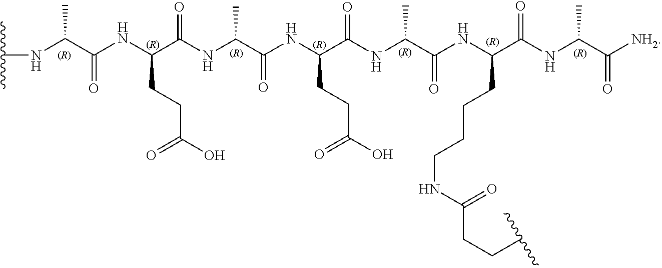

In some embodiments, L¹ is:

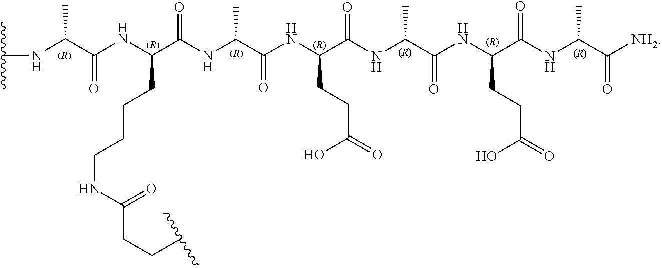

In some embodiments, L¹ is:

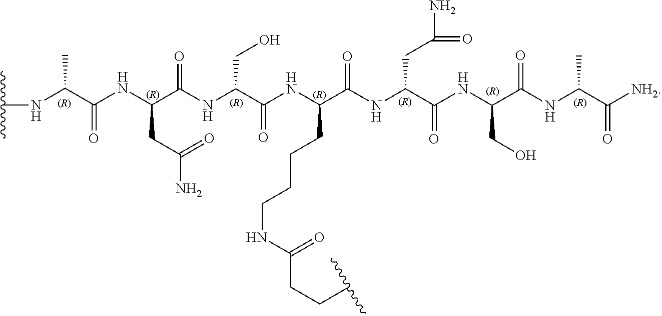

In some embodiments, L¹ is:

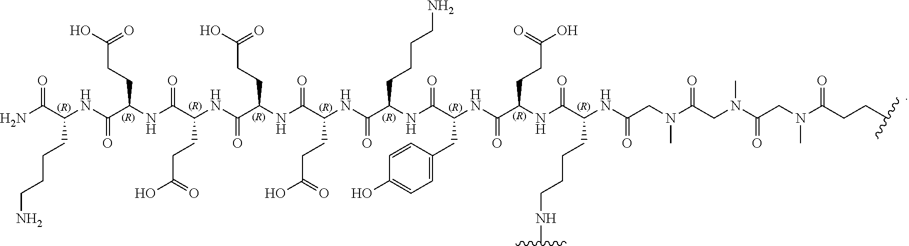

In some embodiments, L¹ is:

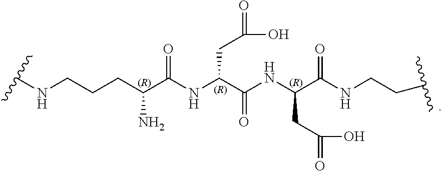

In some embodiments, L¹ is:

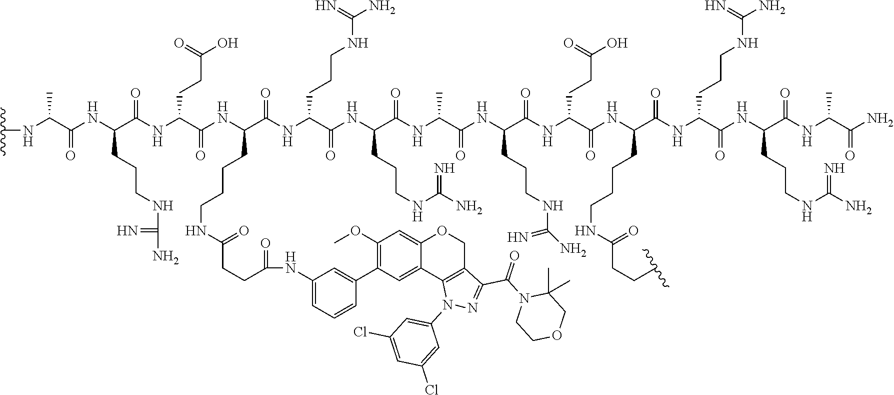 [SMILES: *CCC(=O)NCCCC[C@@H](NC(=O)[C@@H](CCC(=O)O)NC(=O)[C@@H](CCCNC(=N)N)NC(=O)[C@@H](C)NC(=O)[C@@H](CCCNC(=N)N)NC(=O)[C@@H](CCCNC(=N)N)NC(=O)[C@@H](CCCCNC(=O)CCC(=O)Nc1cccc(-c2cc3c(cc2OC)OCc2c(C(=O)N4CCOCC4(C)C)nn(-c4cc(Cl)cc(Cl)c4)c2-3)c1)NC(=O)[C@@H](CCC(=O)O)NC(=O)[C@@H](CCCNC(=N)N)NC(=O)[C@@H](C)N*)C(=O)N[C@H](CCCNC(=N)N)C(=O)N[C@H](CCCNC(=N)N)C(=O)N[C@H](C)C(N)=O]

In some embodiments, -L¹-R⁶ is: —CH₂-R⁶, —CH₂CH₂-R⁶,

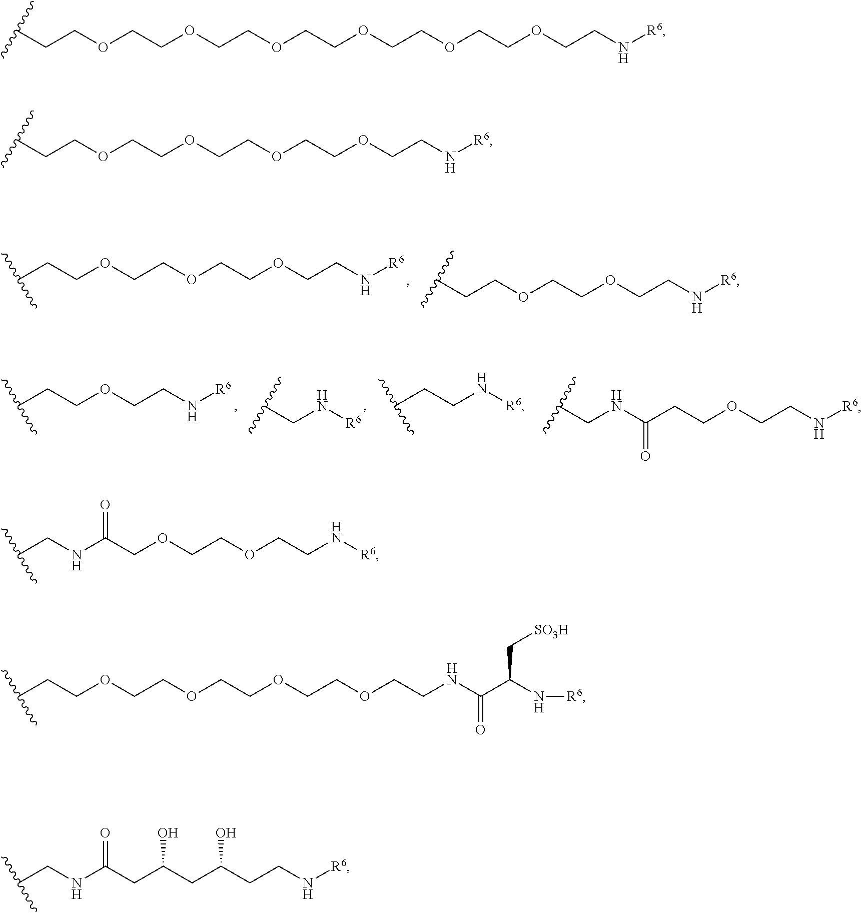

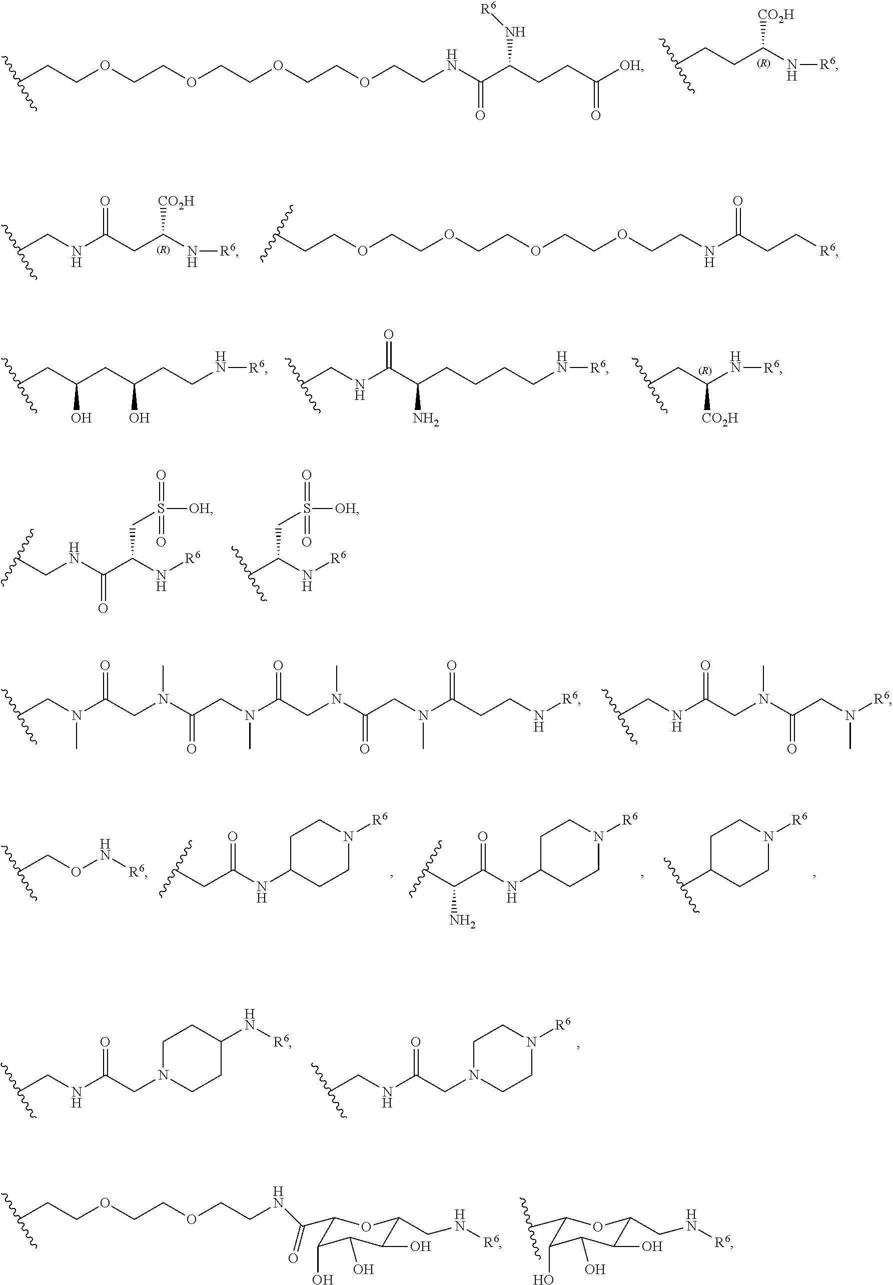

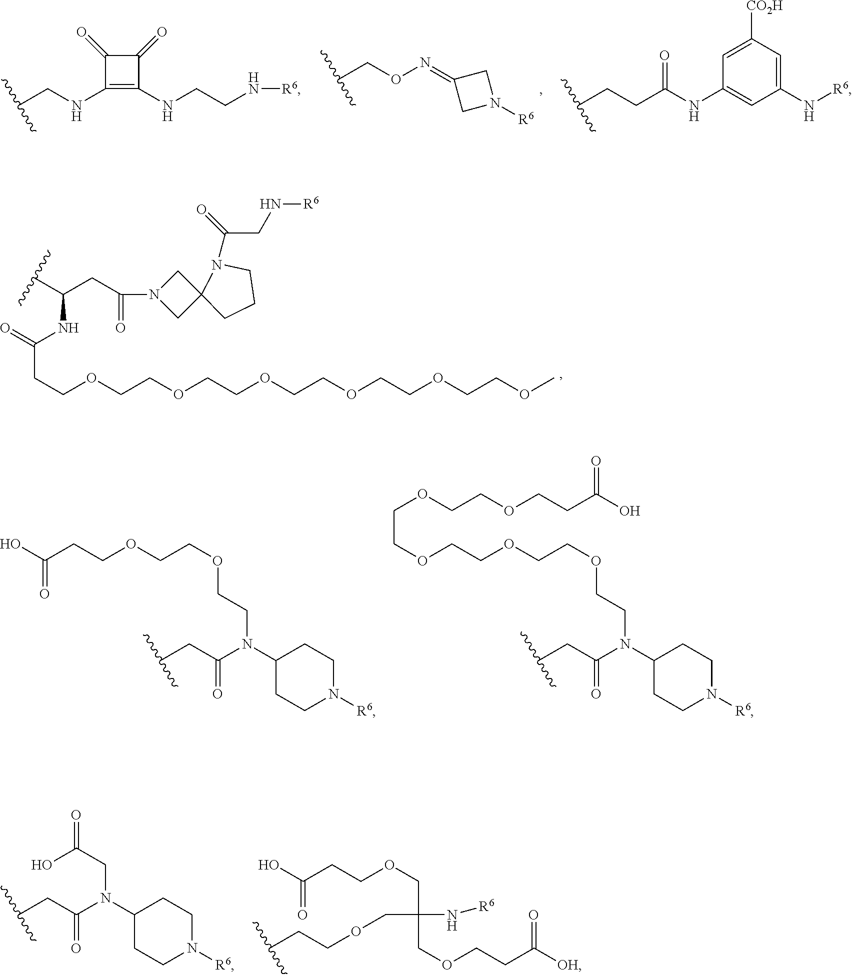

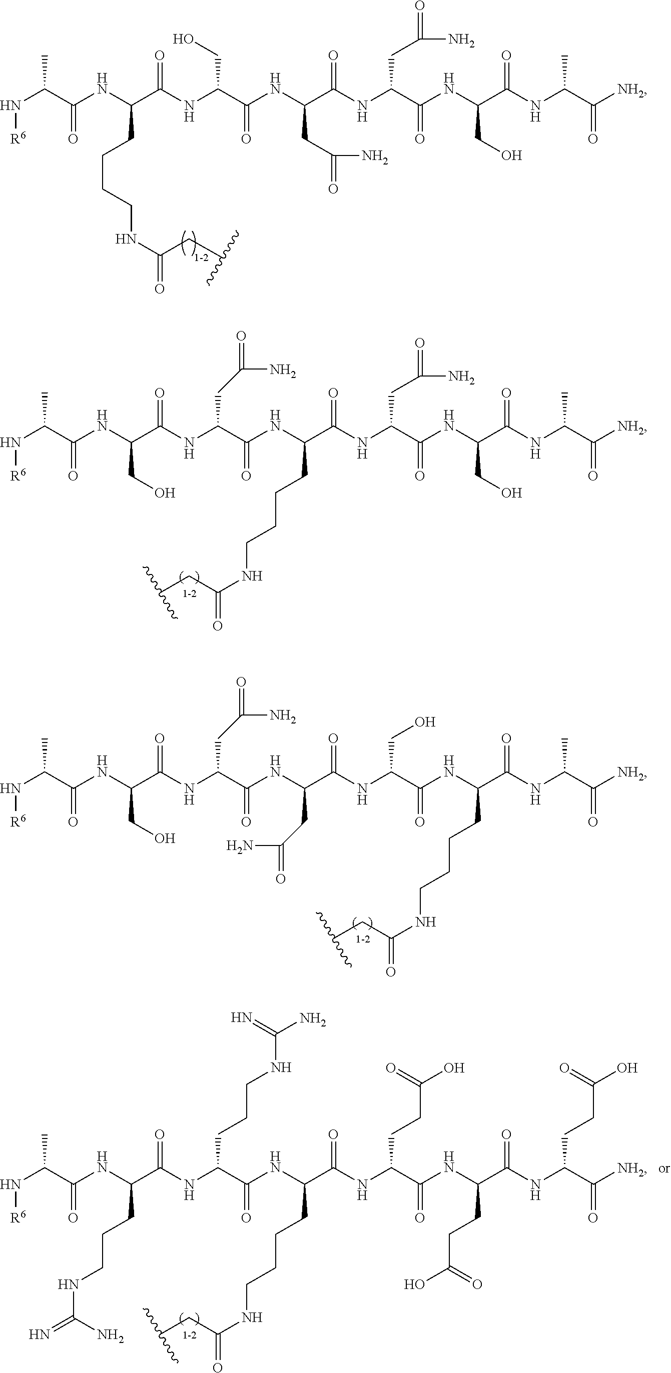

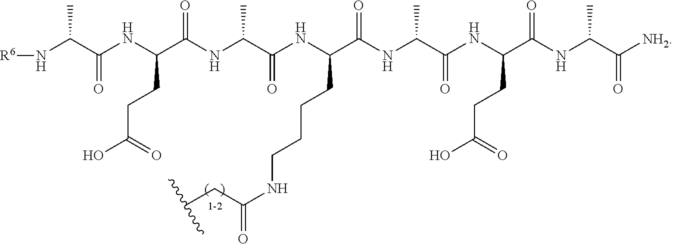

In some embodiments, R is:

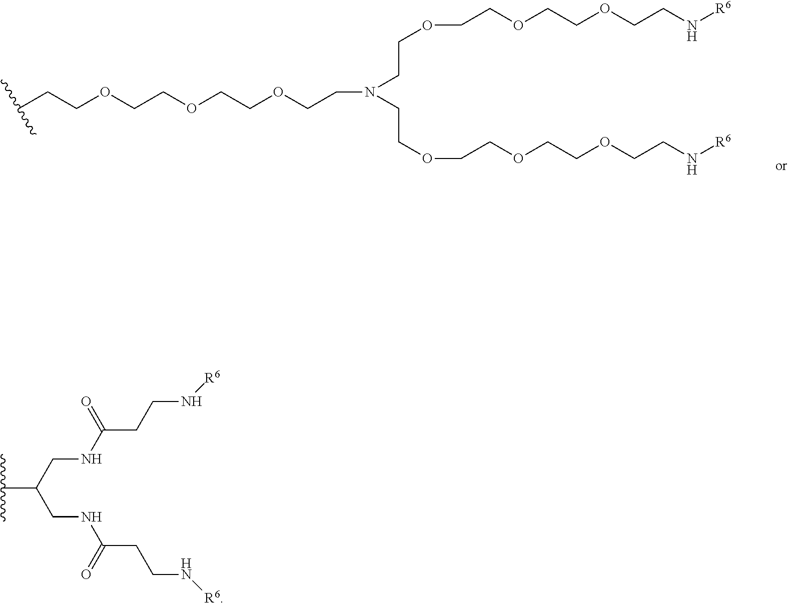

In some embodiments, R is:

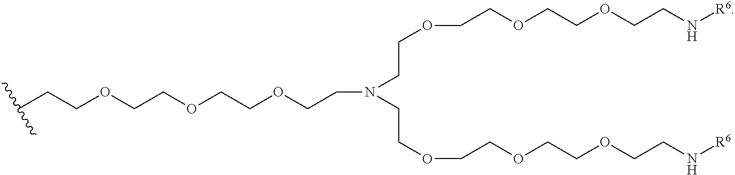

In some embodiments, R is:

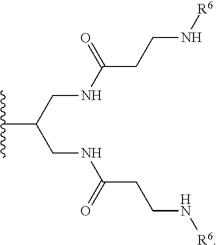

### Representative Linker and Chelating Moieties

In some embodiments, -L¹-R⁶ is:

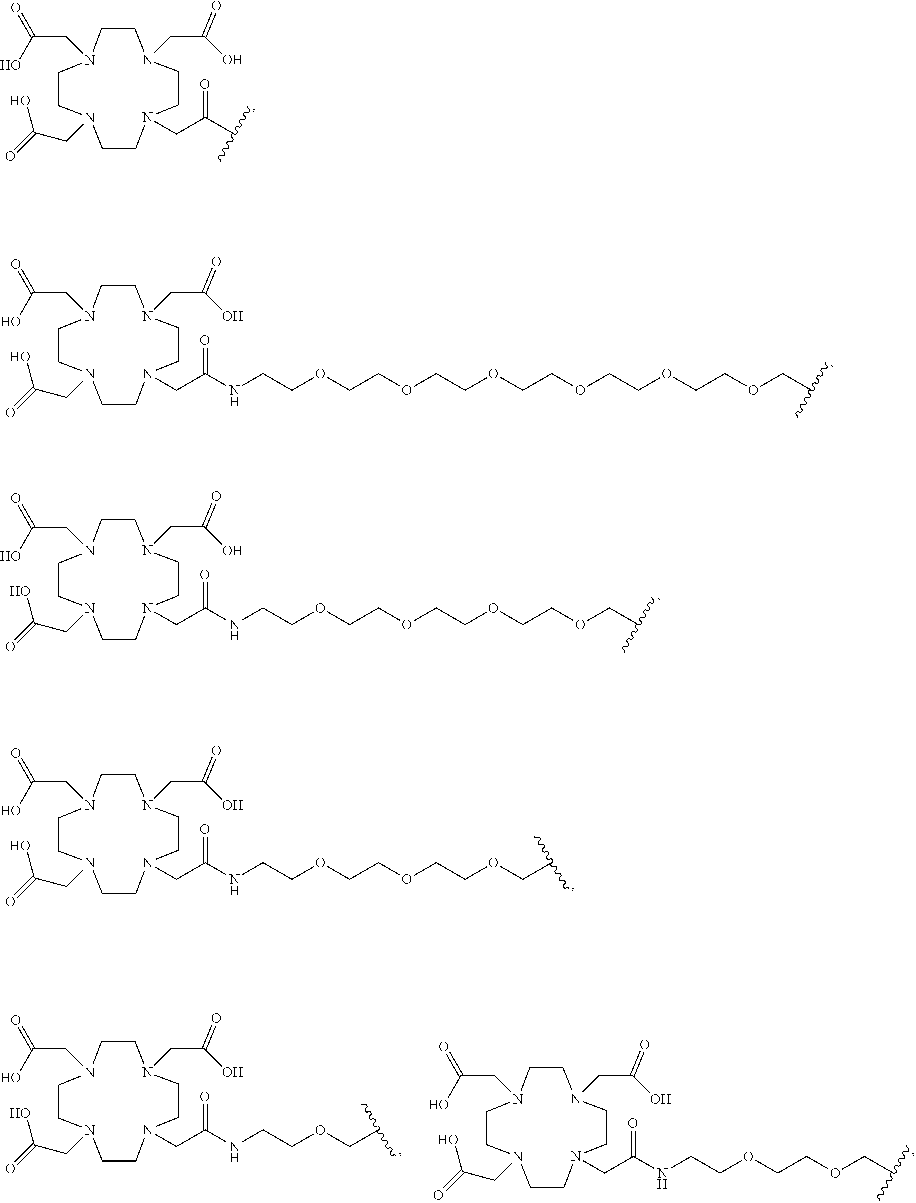 [SMILES: *C(=O)CN1CCN(CC(=O)O)CCN(CC(=O)O)CCN(CC(=O)O)CC1 ; *COCCOCCOCCOCCOCCOCCNC(=O)CN1CCN(CC(=O)O)CCN(CC(=O)O)CCN(CC(=O)O)CC1 ; *COCCOCCOCCOCCNC(=O)CN1CCN(CC(=O)O)CCN(CC(=O)O)CCN(CC(=O)O)CC1 ; *COCCOCCOCCNC(=O)CN1CCN(CC(=O)O)CCN(CC(=O)O)CCN(CC(=O)O)CC1 ; *COCCNC(=O)CN1CCN(CC(=O)O)CCN(CC(=O)O)CCN(CC(=O)O)CC1 ; *COCCOCCNC(=O)CN1CCN(CC(=O)O)CCN(CC(=O)O)CCN(CC(=O)O)CC1]

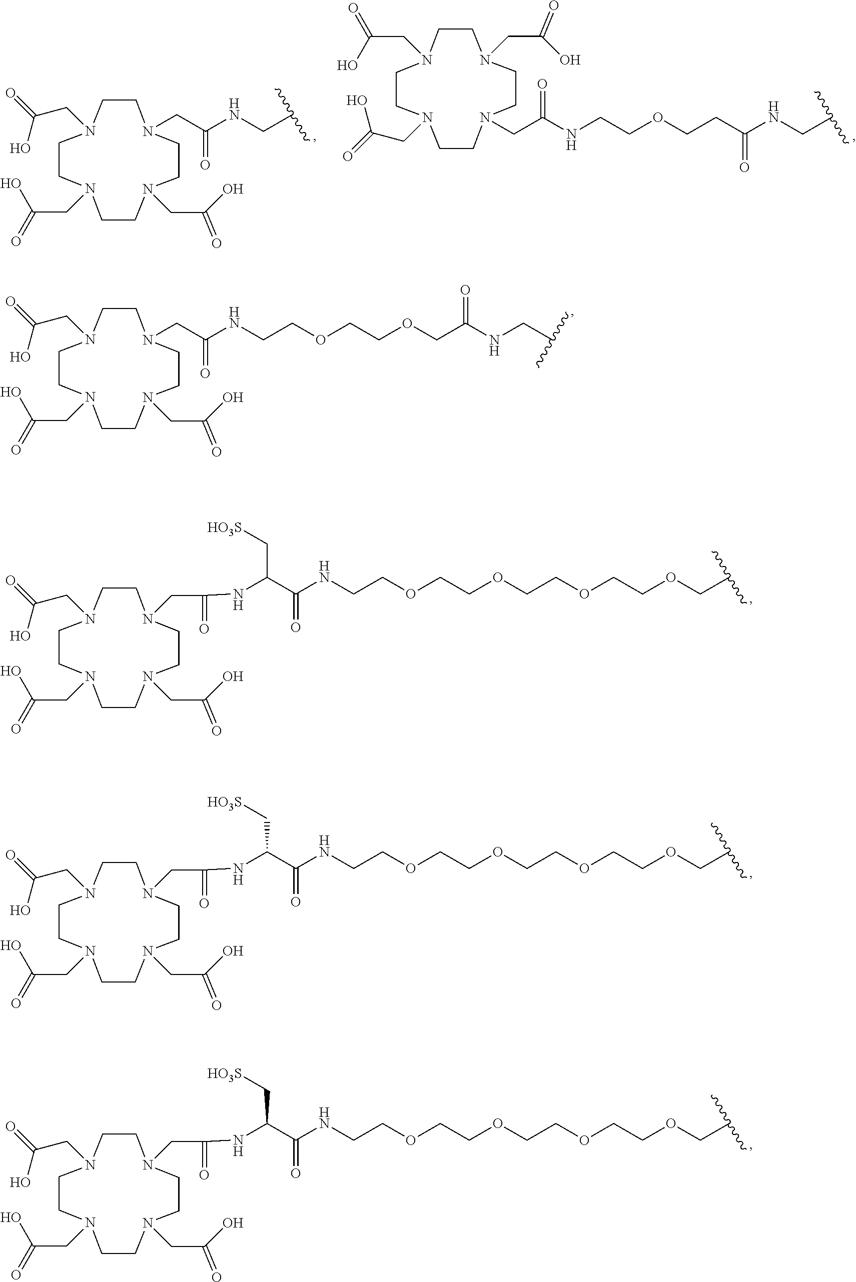 [SMILES: *CNC(=O)CN1CCN(CC(=O)O)CCN(CC(=O)O)CCN(CC(=O)O)CC1 ; *CNC(=O)CCOCCNC(=O)CN1CCN(CC(=O)O)CCN(CC(=O)O)CCN(CC(=O)O)CC1 ; *CNC(=O)COCCOCCNC(=O)CN1CCN(CC(=O)O)CCN(CC(=O)O)CCN(CC(=O)O)CC1 ; *COCCOCCOCCOCCNC(=O)C(CS(=O)(=O)O)NC(=O)CN1CCN(CC(=O)O)CCN(CC(=O)O)CCN(CC(=O)O)CC1 ; *COCCOCCOCCOCCNC(=O)[C@@H](CS(=O)(=O)O)NC(=O)CN1CCN(CC(=O)O)CCN(CC(=O)O)CCN(CC(=O)O)CC1 ; *COCCOCCOCCOCCNC(=O)[C@H](CS(=O)(=O)O)NC(=O)CN1CCN(CC(=O)O)CCN(CC(=O)O)CCN(CC(=O)O)CC1]

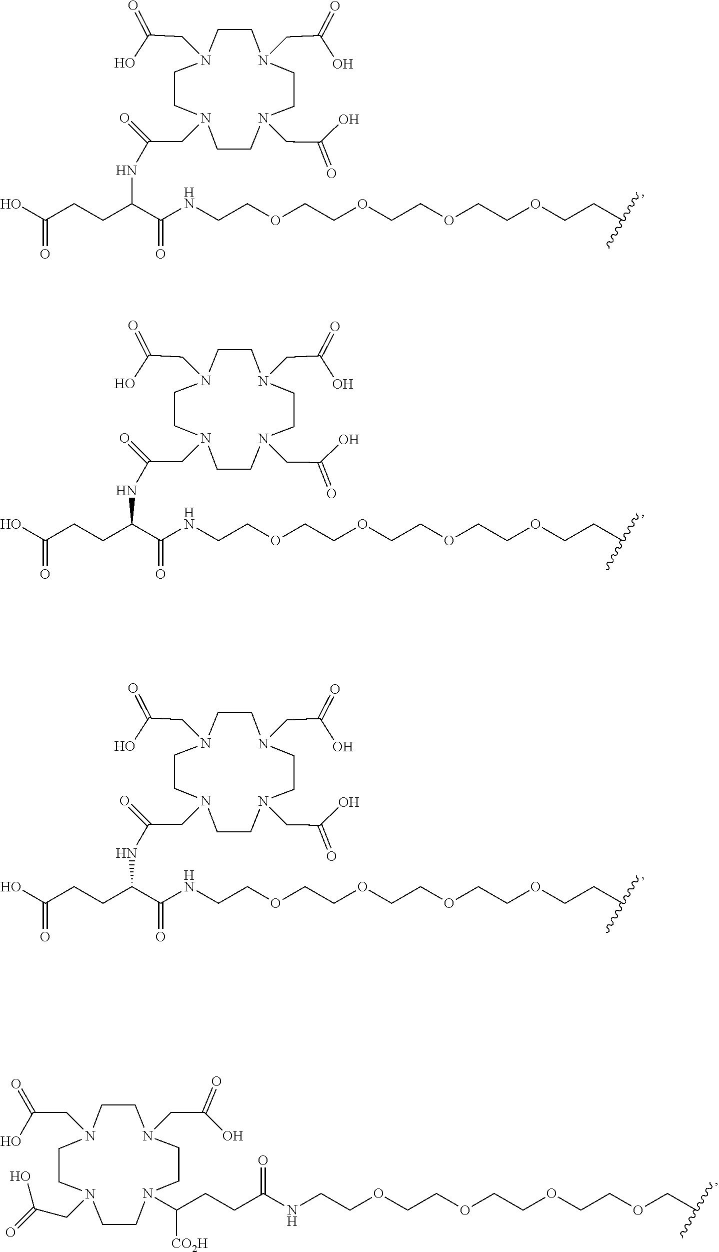 [SMILES: *CCOCCOCCOCCOCCNC(=O)C(CCC(=O)O)NC(=O)CN1CCN(CC(=O)O)CCN(CC(=O)O)CCN(CC(=O)O)CC1 ; *CCOCCOCCOCCOCCNC(=O)[C@@H](CCC(=O)O)NC(=O)CN1CCN(CC(=O)O)CCN(CC(=O)O)CCN(CC(=O)O)CC1 ; *CCOCCOCCOCCOCCNC(=O)[C@H](CCC(=O)O)NC(=O)CN1CCN(CC(=O)O)CCN(CC(=O)O)CCN(CC(=O)O)CC1 ; *COCCOCCOCCOCCNC(=O)CCC(C(=O)O)N1CCN(CC(=O)O)CCN(CC(=O)O)CCN(CC(=O)O)CC1]

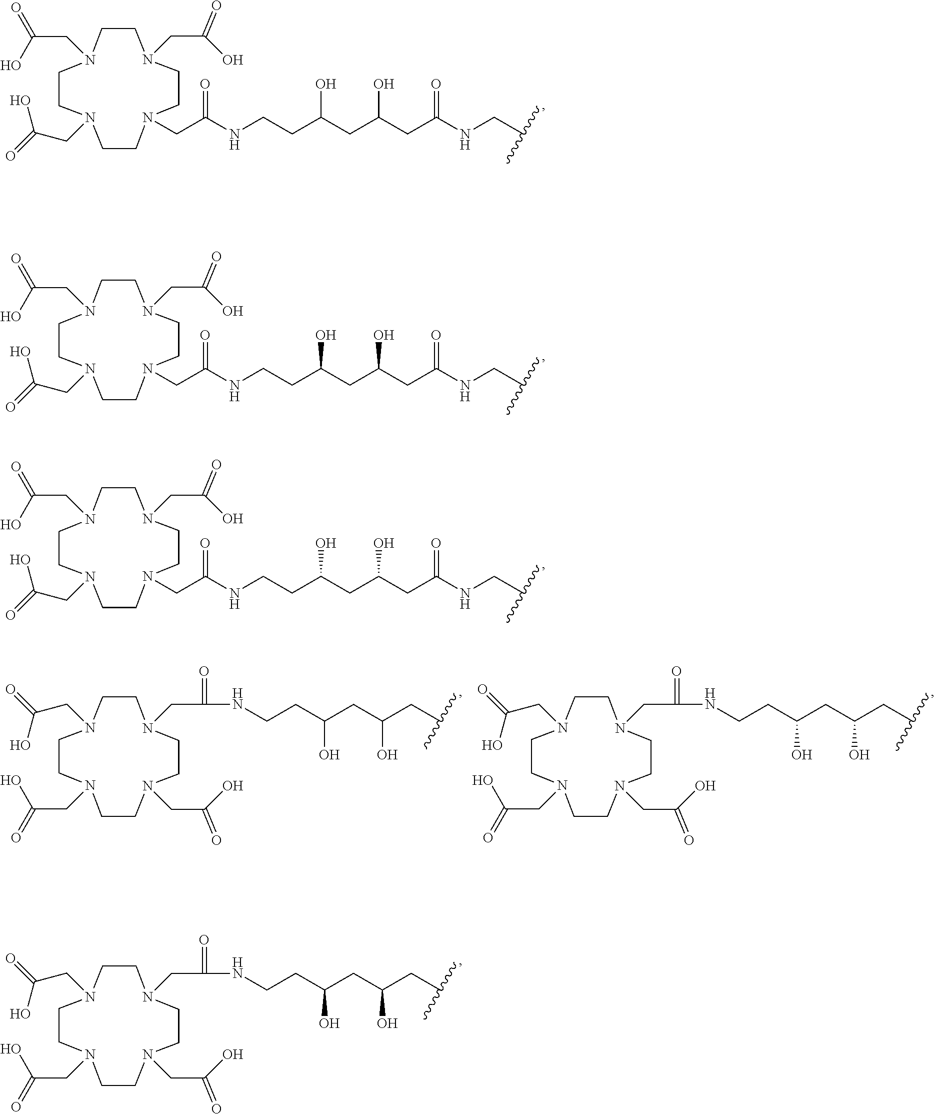 [SMILES: *CNC(=O)CC(O)CC(O)CCNC(=O)CN1CCN(CC(=O)O)CCN(CC(=O)O)CCN(CC(=O)O)CC1 ; *CNC(=O)C[C@H](O)C[C@H](O)CCNC(=O)CN1CCN(CC(=O)O)CCN(CC(=O)O)CCN(CC(=O)O)CC1 ; *CNC(=O)C[C@@H](O)C[C@@H](O)CCNC(=O)CN1CCN(CC(=O)O)CCN(CC(=O)O)CCN(CC(=O)O)CC1 ; *CC(O)CC(O)CCNC(=O)CN1CCN(CC(=O)O)CCN(CC(=O)O)CCN(CC(=O)O)CC1 ; *C[C@H](O)C[C@H](O)CCNC(=O)CN1CCN(CC(=O)O)CCN(CC(=O)O)CCN(CC(=O)O)CC1 ; *C[C@@H](O)C[C@@H](O)CCNC(=O)CN1CCN(CC(=O)O)CCN(CC(=O)O)CCN(CC(=O)O)CC1]

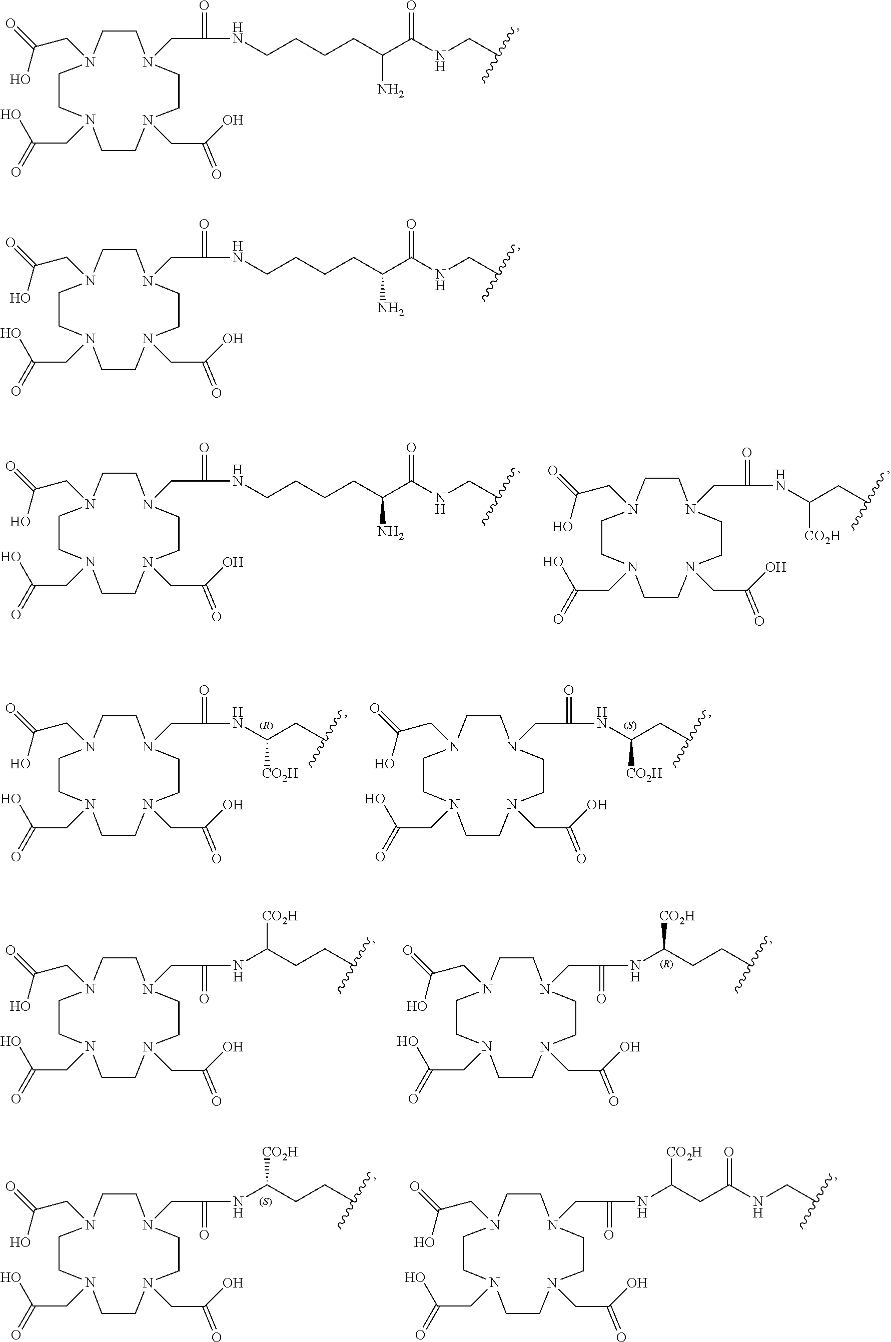

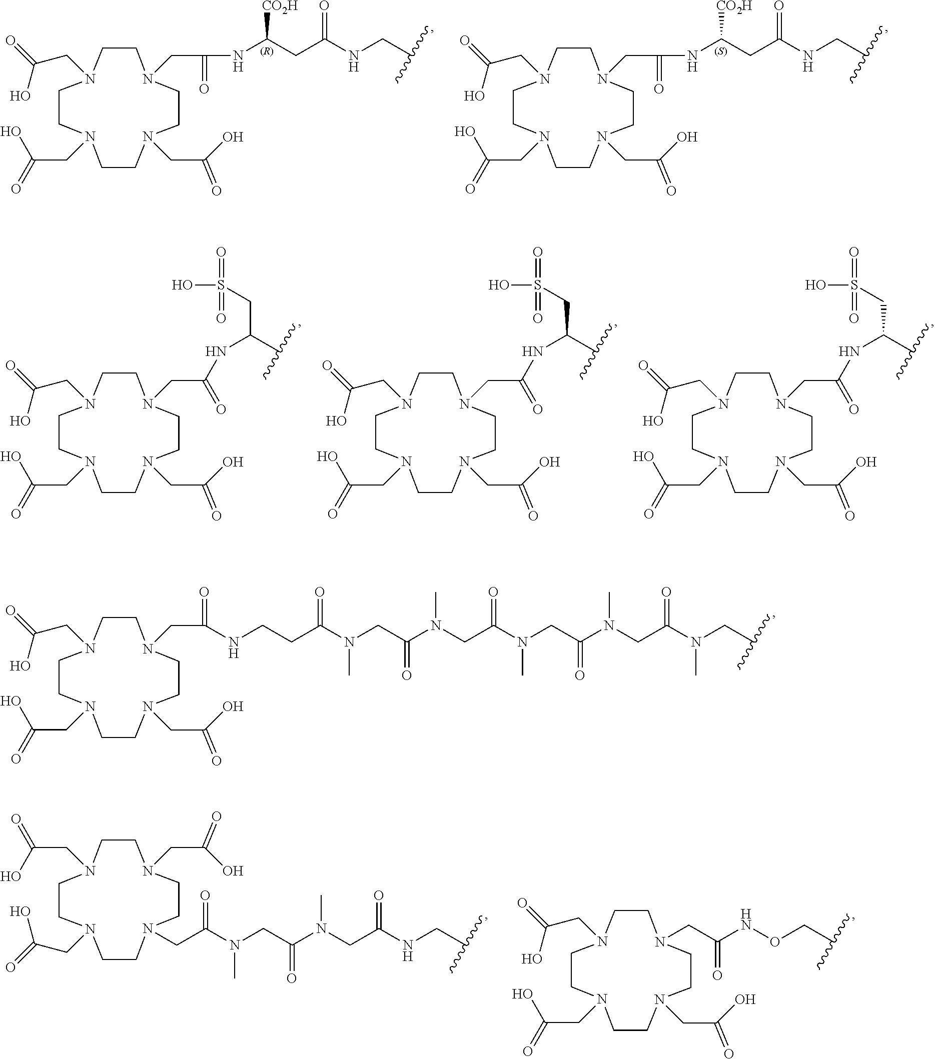

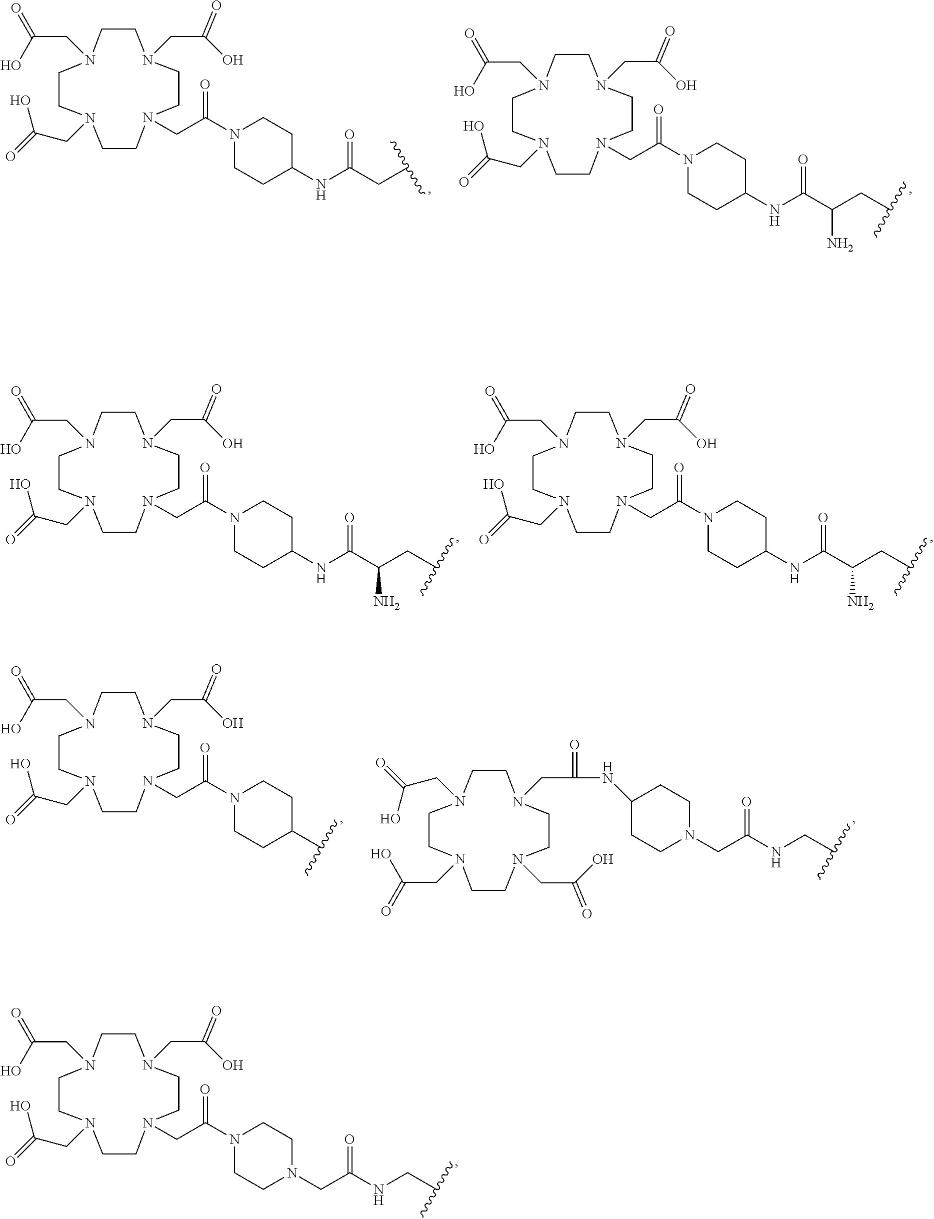

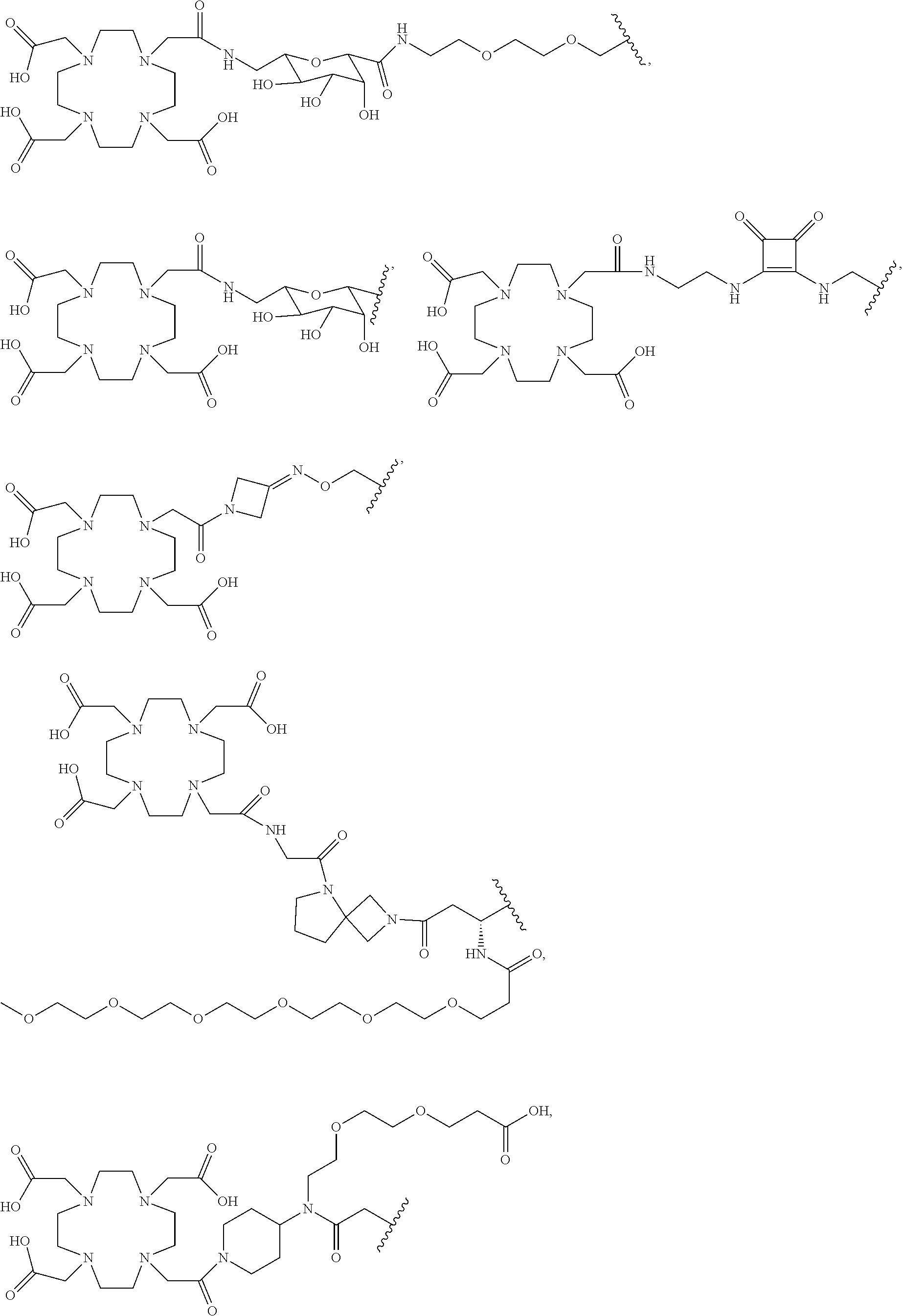 [SMILES: *COCCOCCNC(=O)C1OC(CNC(=O)CN2CCN(CC(=O)O)CCN(CC(=O)O)CCN(CC(=O)O)CC2)C(O)C(O)C1O ; *C1OC(CNC(=O)CN2CCN(CC(=O)O)CCN(CC(=O)O)CCN(CC(=O)O)CC2)C(O)C(O)C1O ; *CNc1c(NCCNC(=O)CN2CCN(CC(=O)O)CCN(CC(=O)O)CCN(CC(=O)O)CC2)c(=O)c1=O ; *CON=C1CN(C(=O)CN2CCN(CC(=O)O)CCN(CC(=O)O)CCN(CC(=O)O)CC2)C1 ; *[C@@H](CC(=O)N1CC2(CCCN2C(=O)CNC(=O)CN2CCN(CC(=O)O)CCN(CC(=O)O)CCN(CC(=O)O)CC2)C1)NC(=O)CCOCCOCCOCCOCCOCCOC ; *CC(=O)N(CCOCCOCCC(=O)O)C1CCN(C(=O)CN2CCN(CC(=O)O)CCN(CC(=O)O)CCN(CC(=O)O)CC2)CC1]

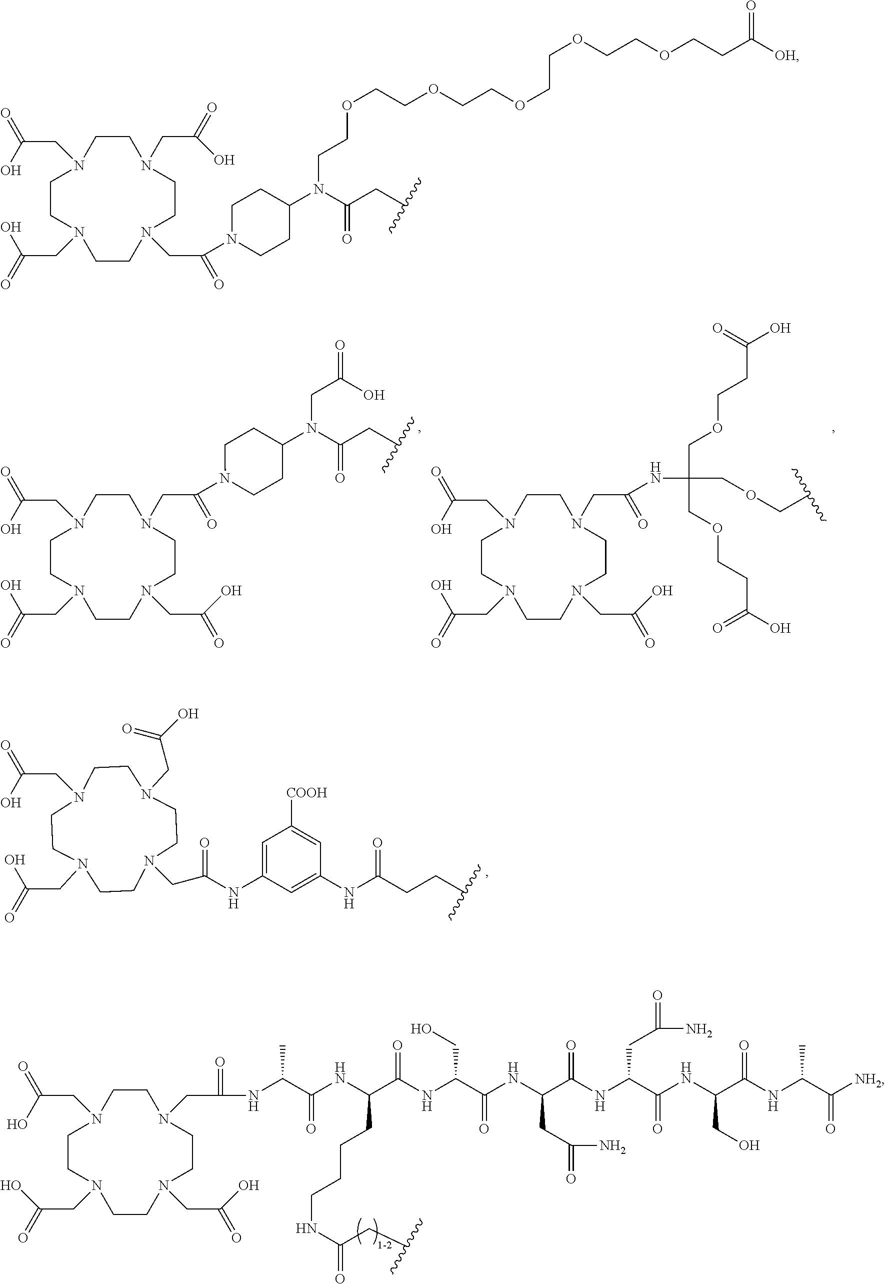

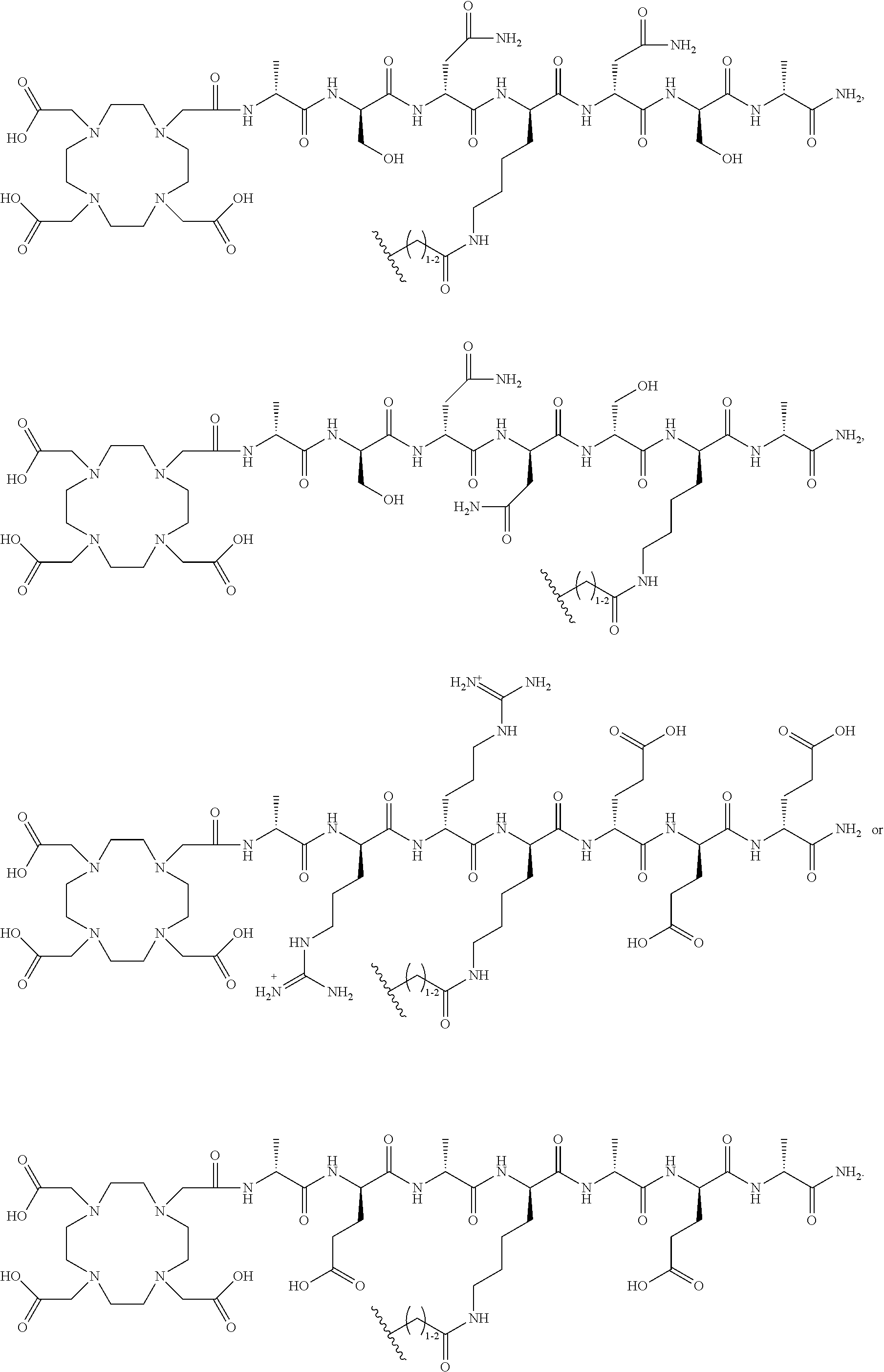

In some embodiments, -L¹-R⁶ is:

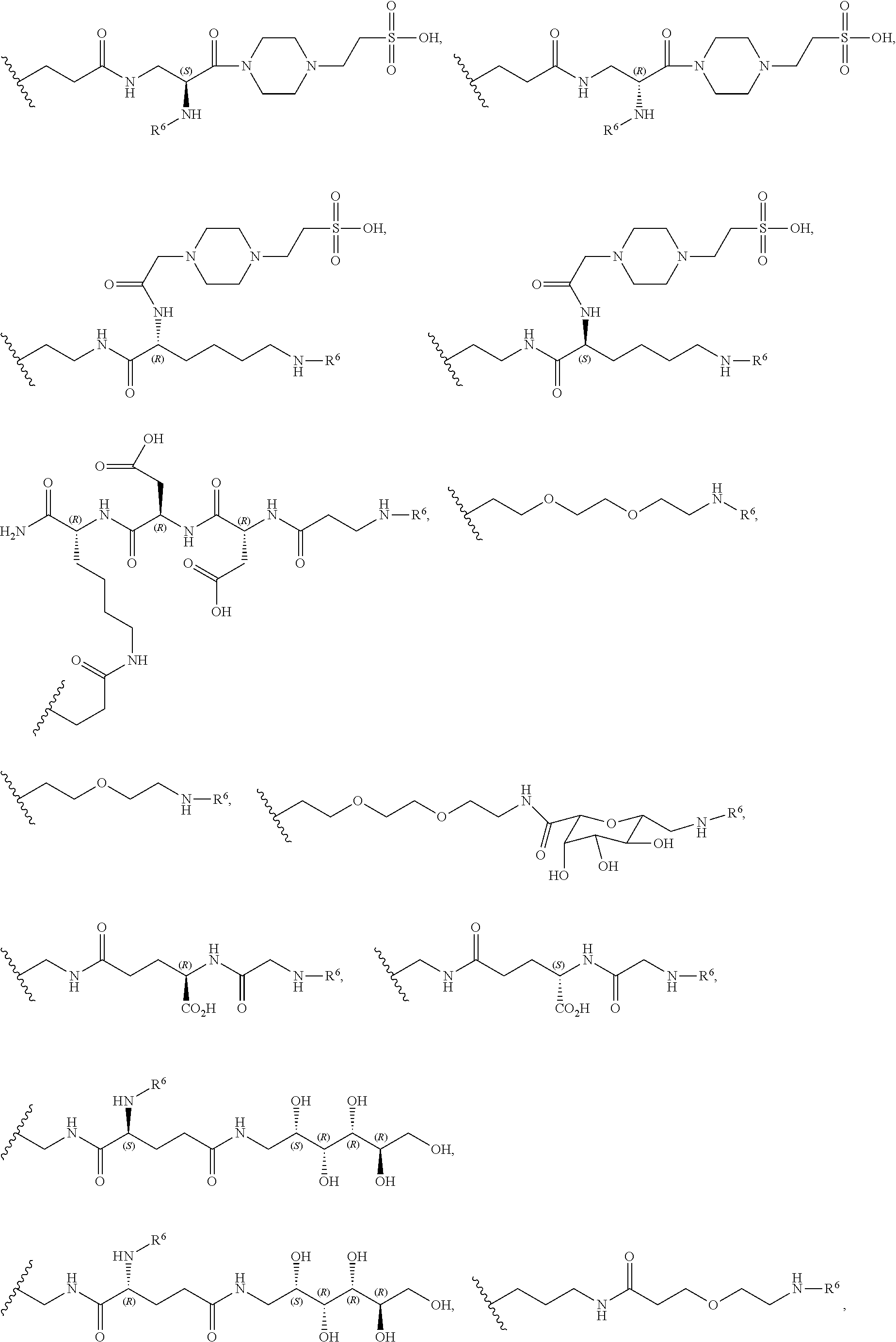

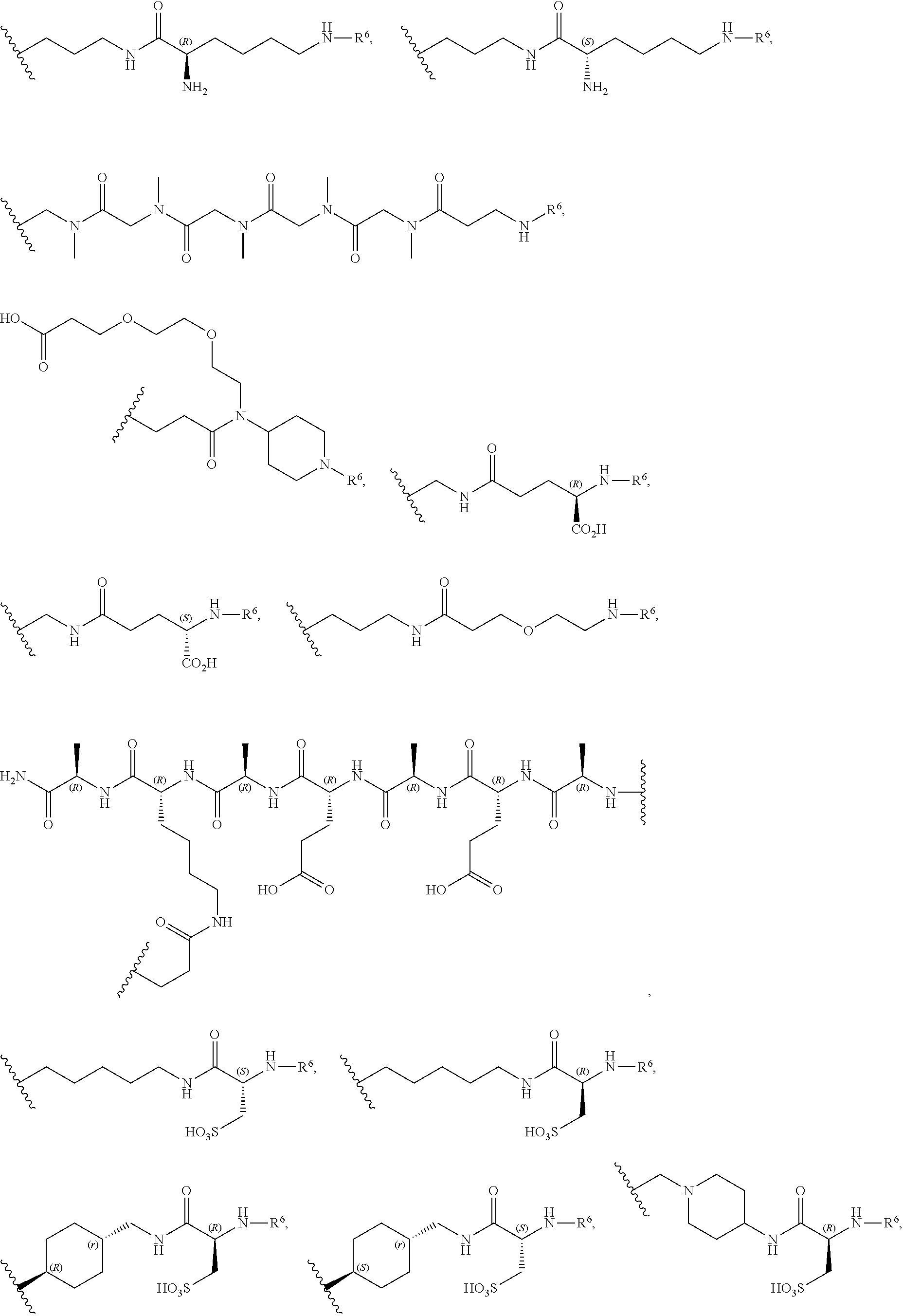

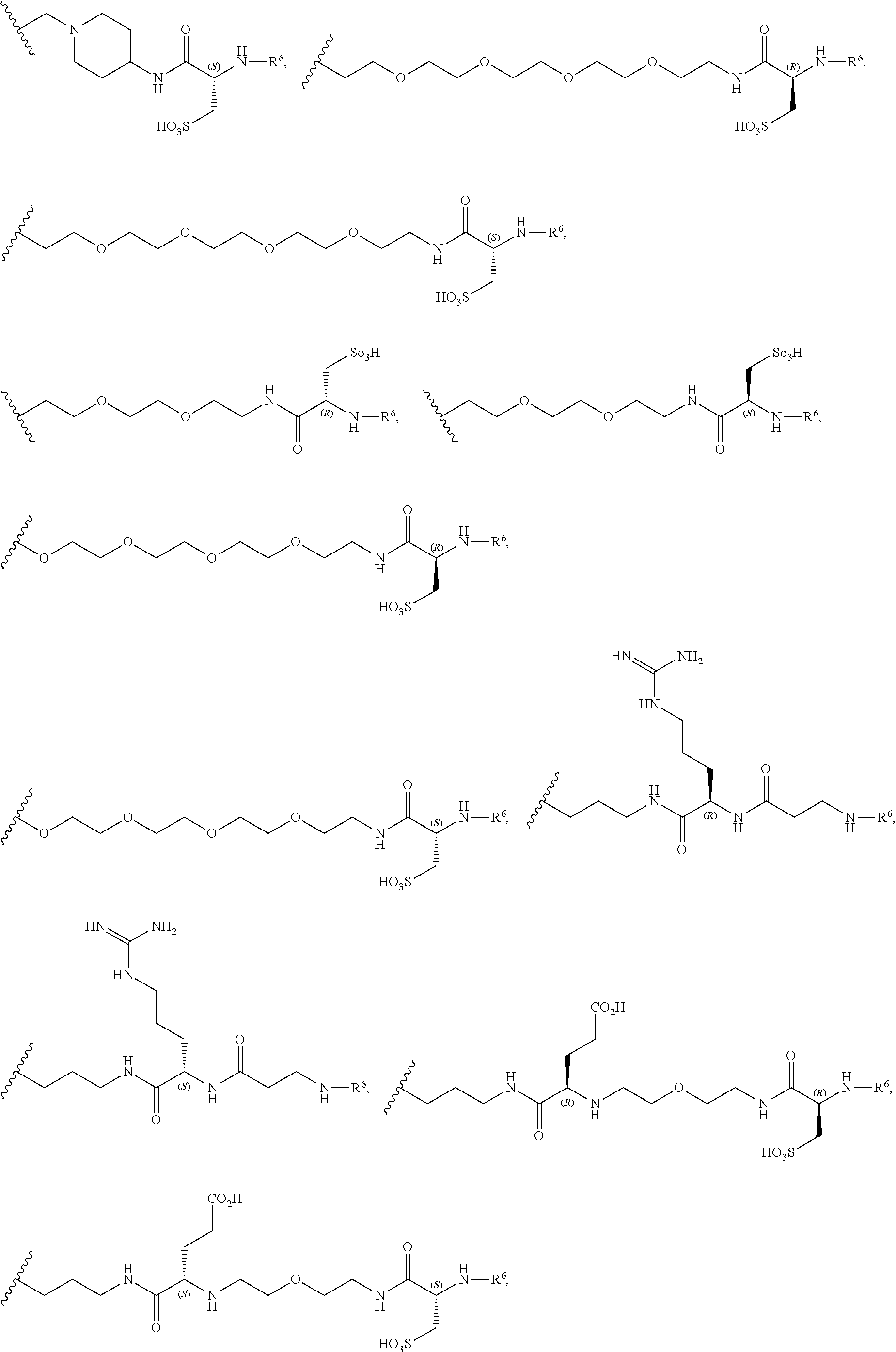

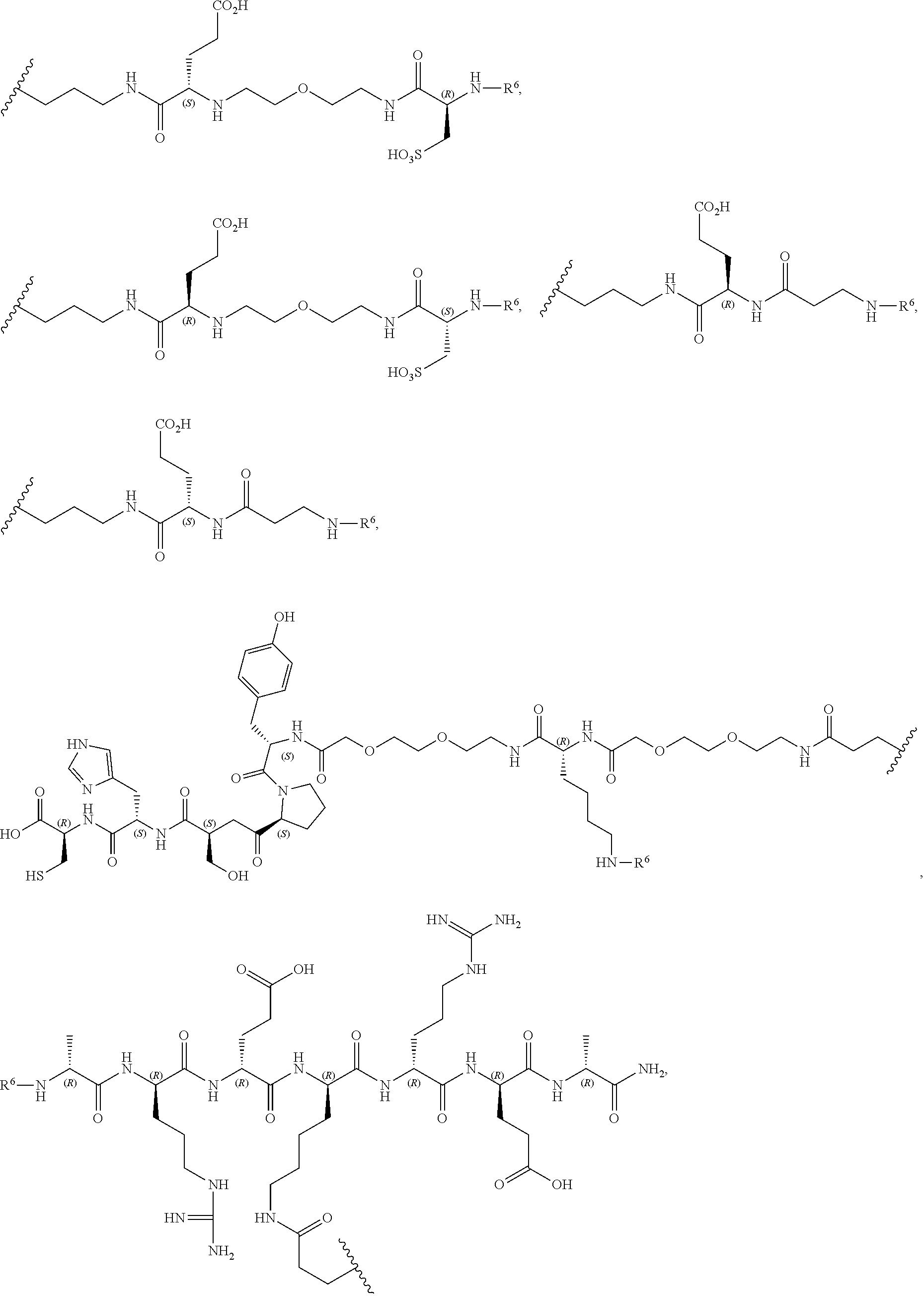

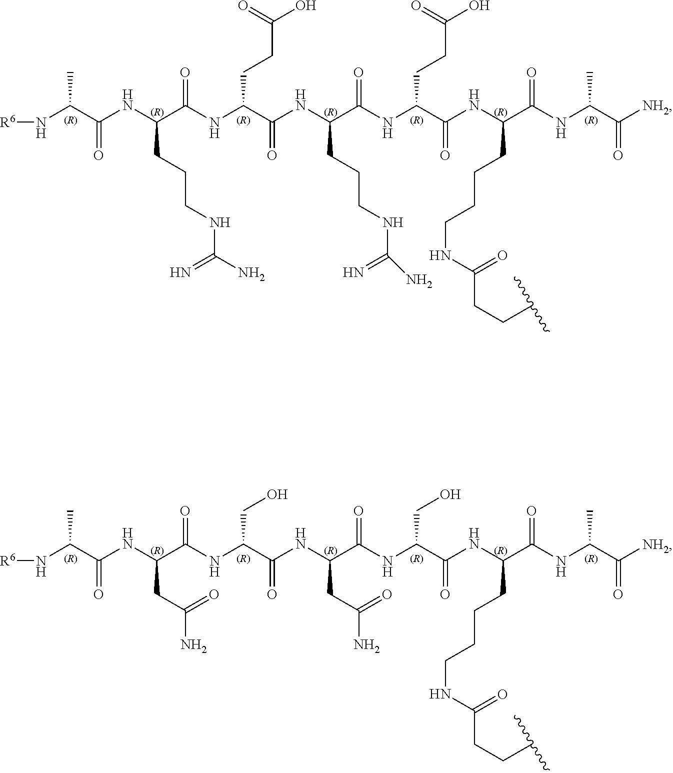

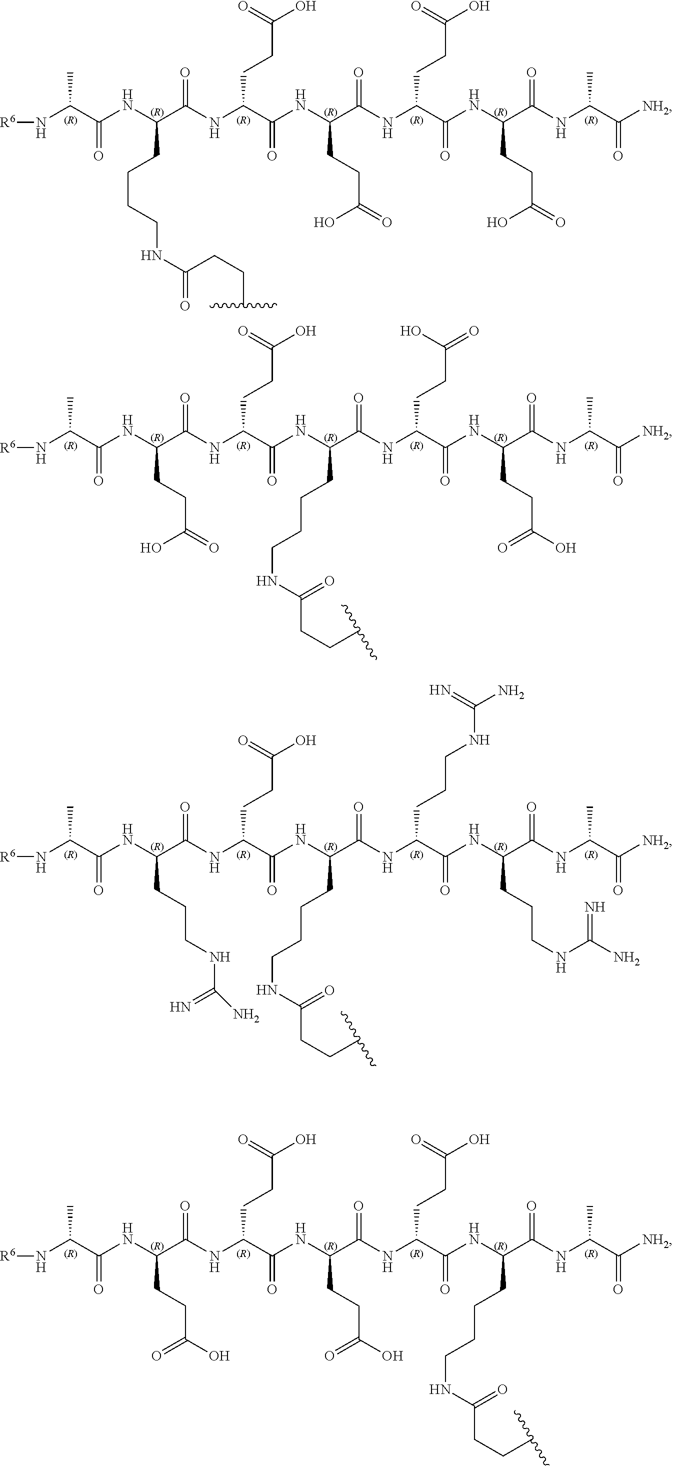

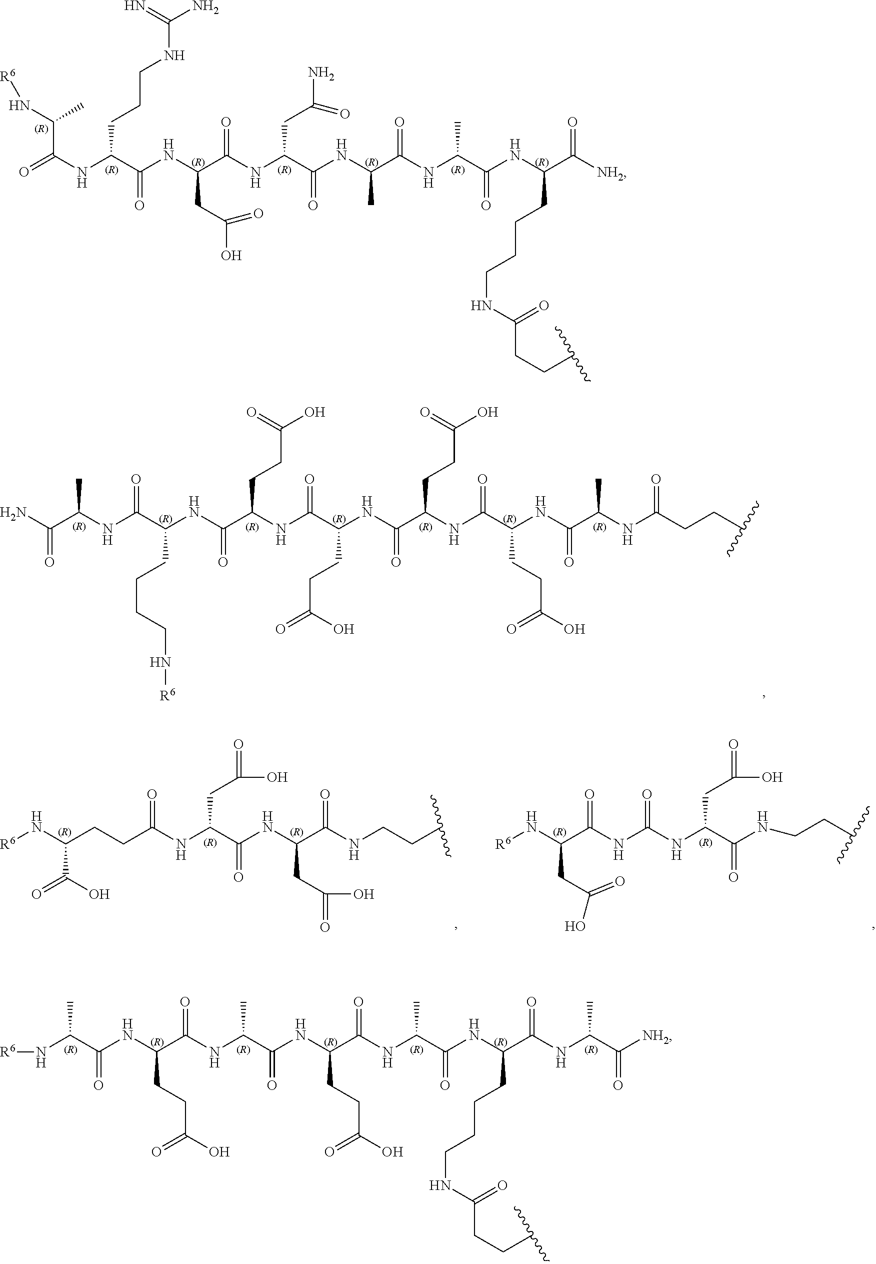

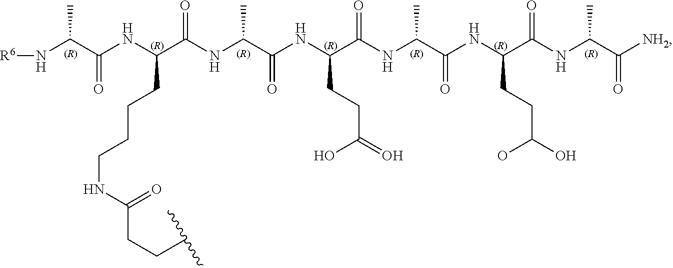

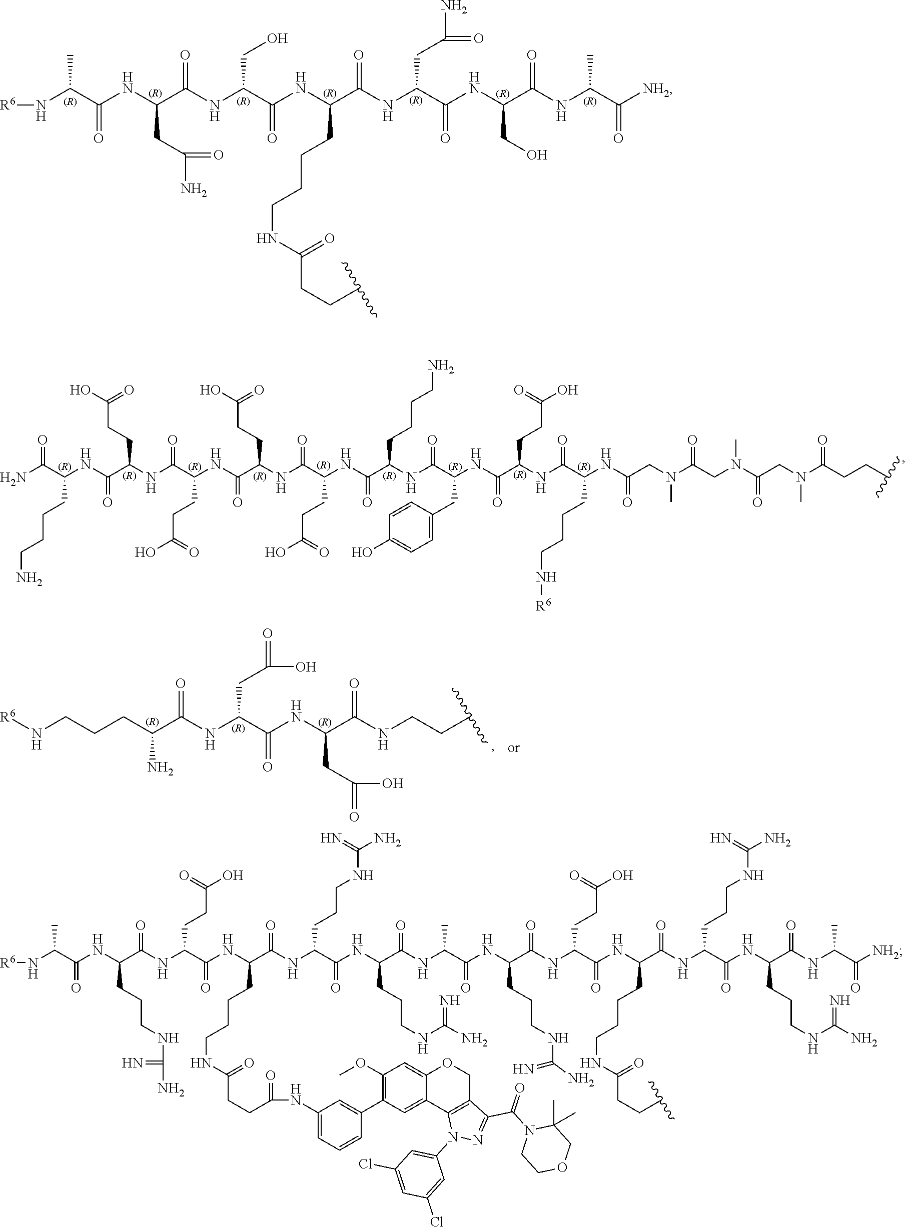

wherein R⁶ is

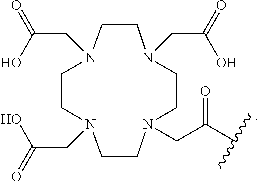 [SMILES: *C(=O)CN1CCN(CC(=O)O)CCN(CC(=O)O)CCN(CC(=O)O)CC1]

In some embodiments, R is:

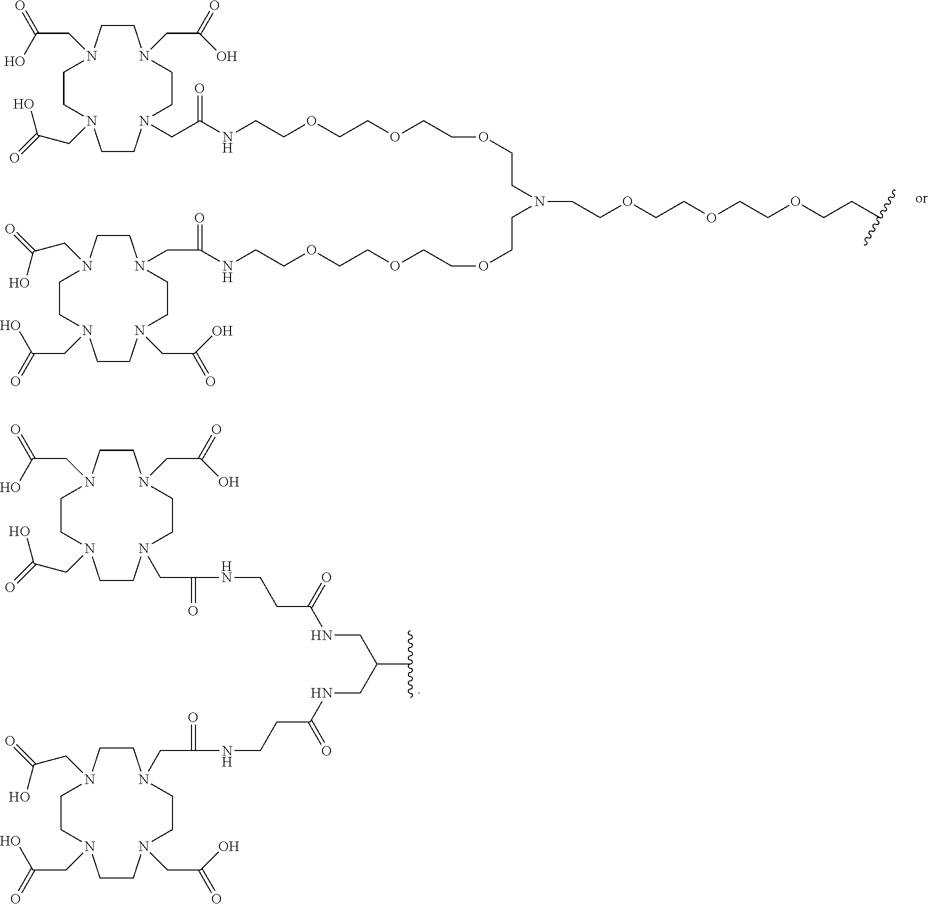 [SMILES: *CCOCCOCCOCCN(CCOCCOCCOCCNC(=O)CN1CCN(CC(=O)O)CCN(CC(=O)O)CCN(CC(=O)O)CC1)CCOCCOCCOCCNC(=O)CN1CCN(CC(=O)O)CCN(CC(=O)O)CCN(CC(=O)O)CC1 ; *C(CNC(=O)CCNC(=O)CN1CCN(CC(=O)O)CCN(CC(=O)O)CCN(CC(=O)O)CC1)CNC(=O)CCNC(=O)CN1CCN(CC(=O)O)CCN(CC(=O)O)CCN(CC(=O)O)CC1]

### Representative Compounds

In some embodiments, the compound of Formula (I) has one of the following structures, or a pharmaceutically acceptable salt thereof:

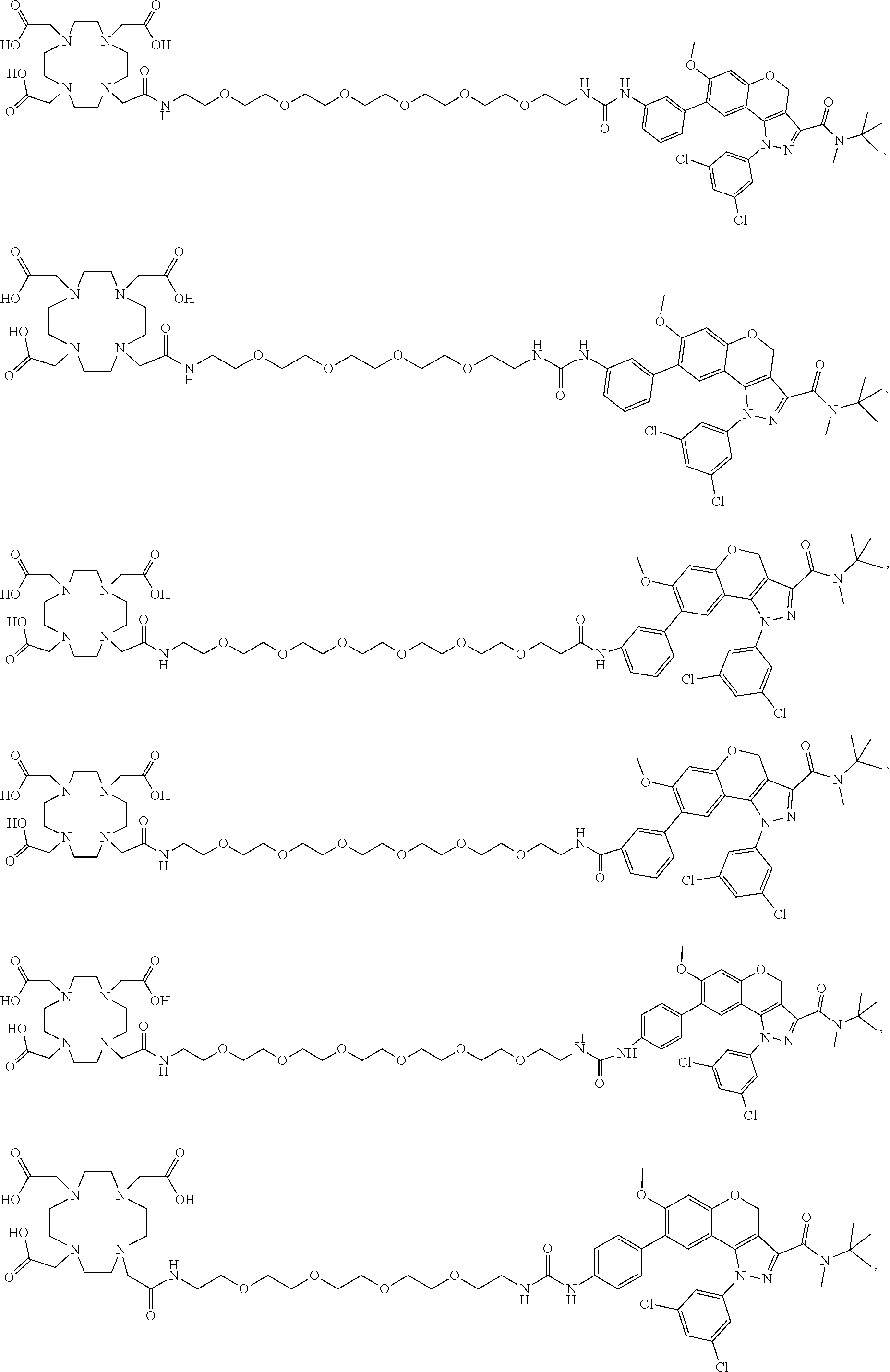

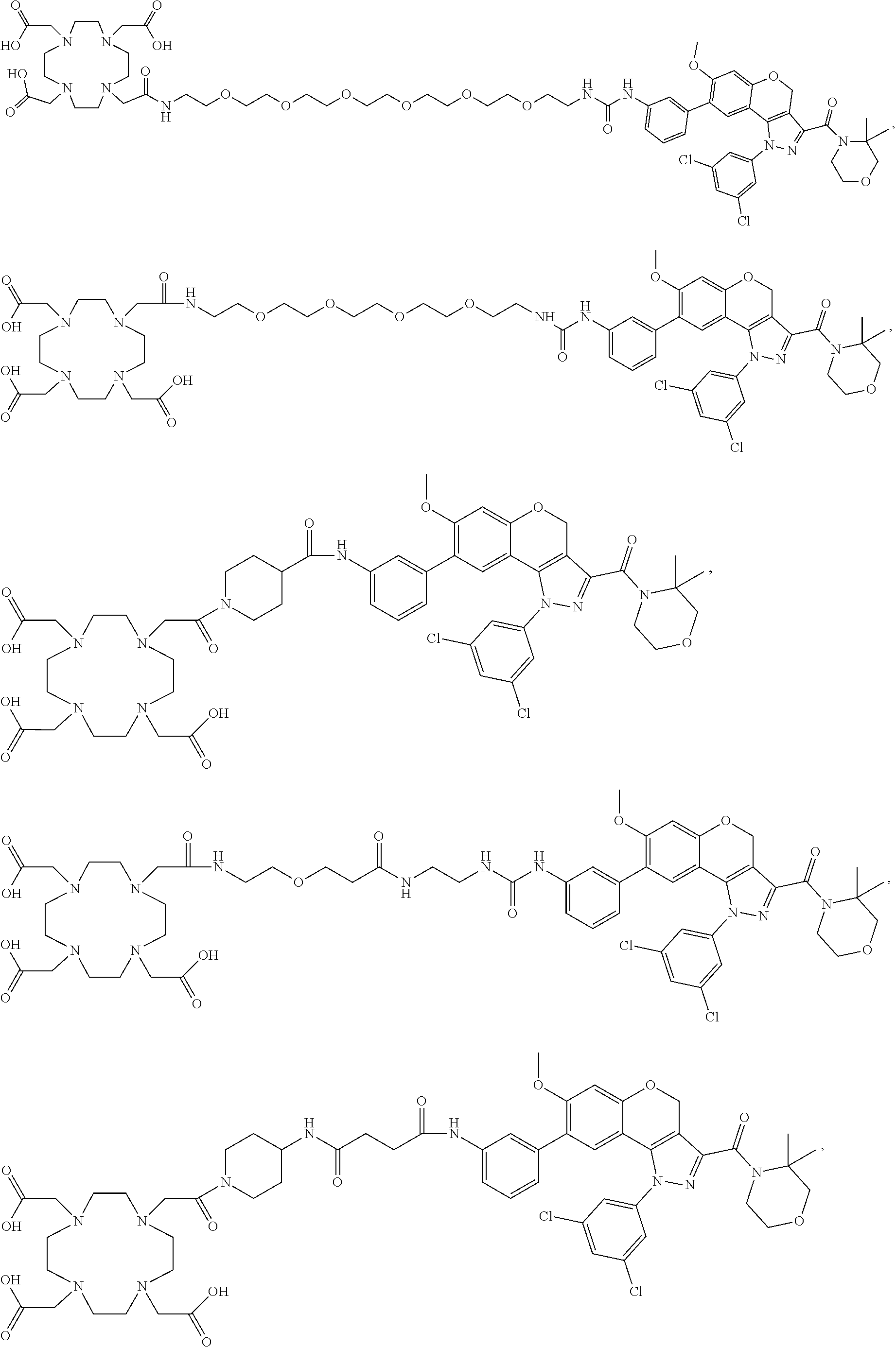

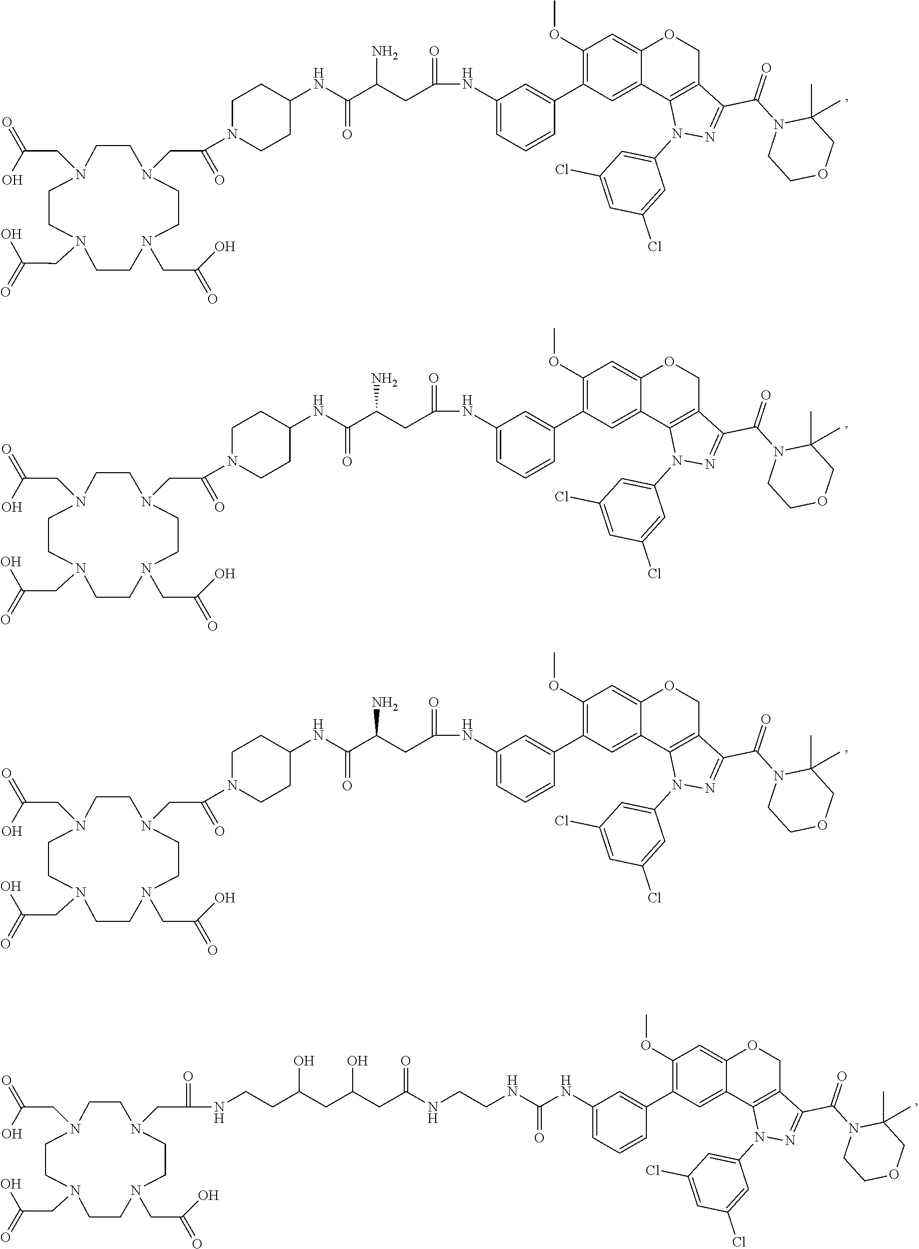

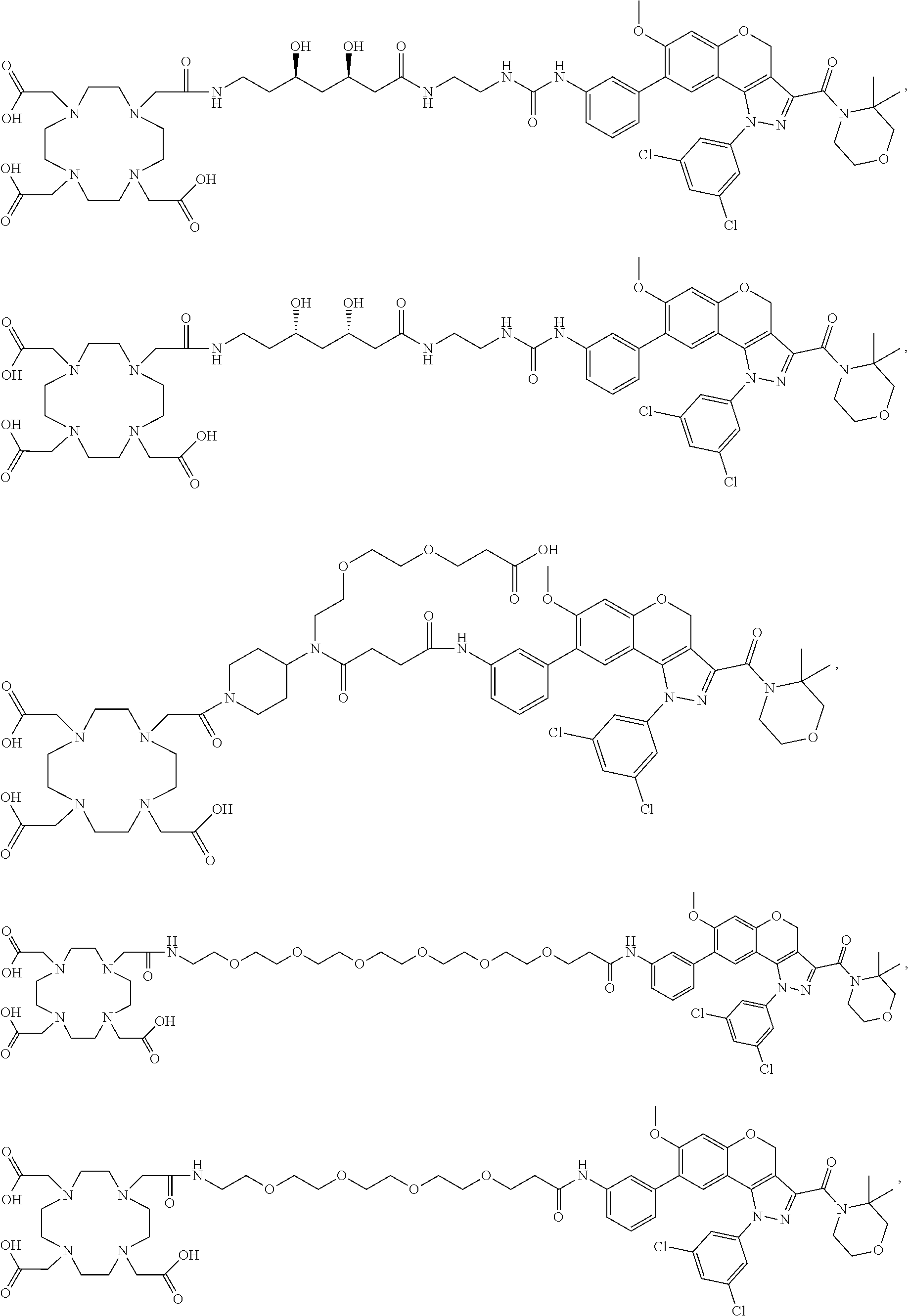

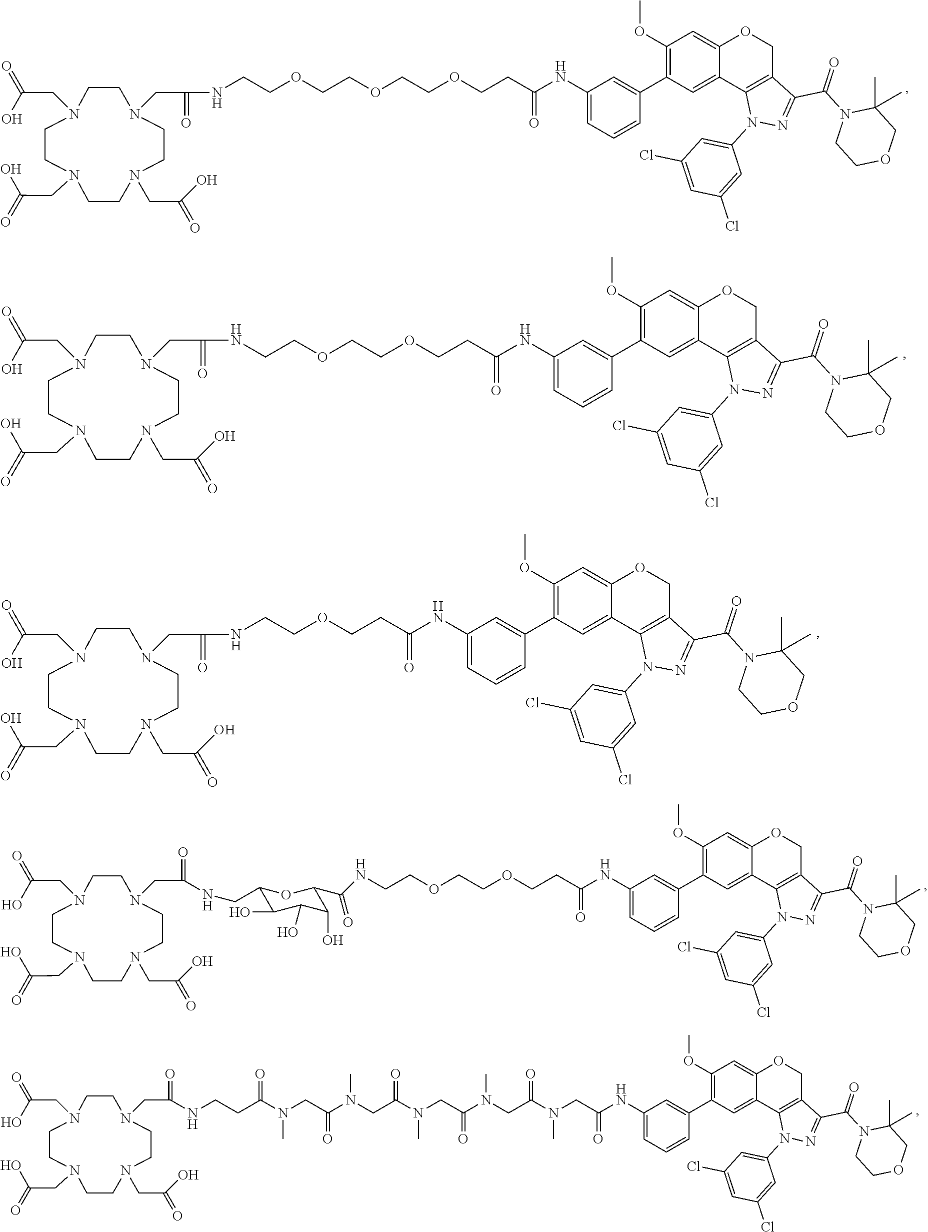

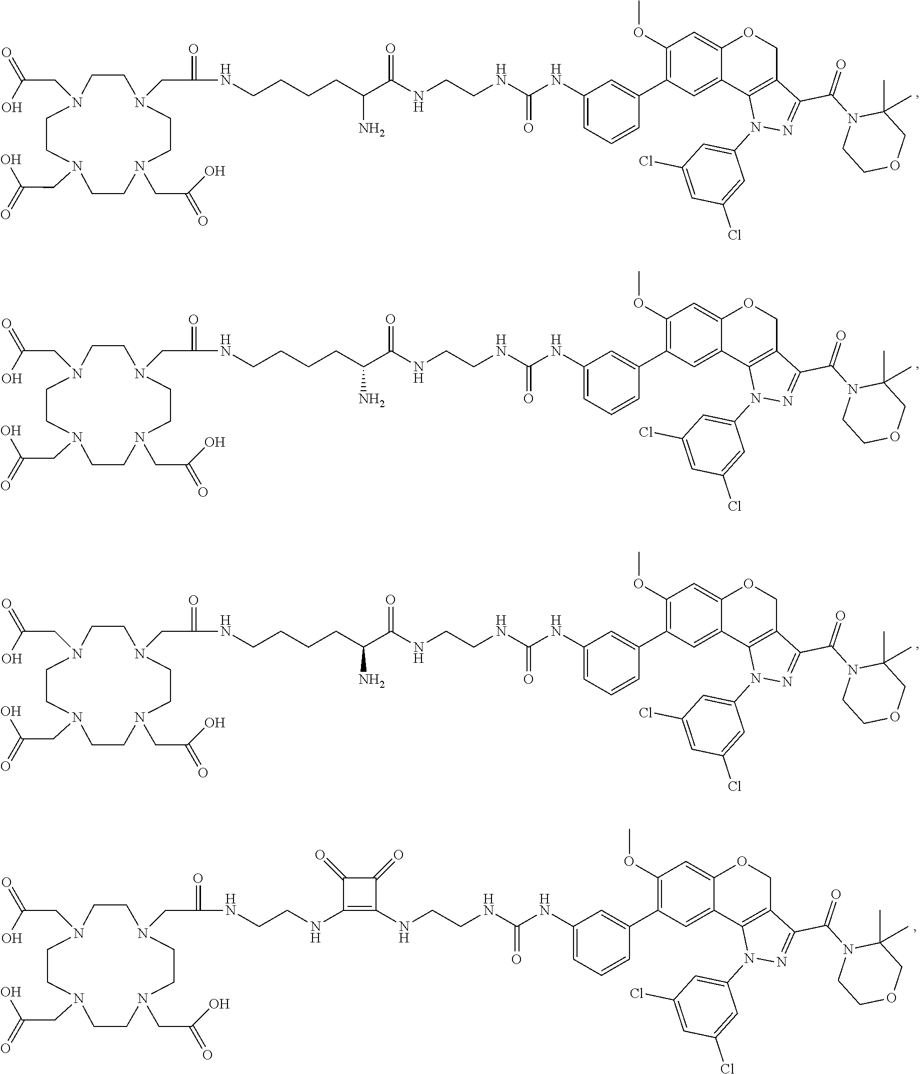

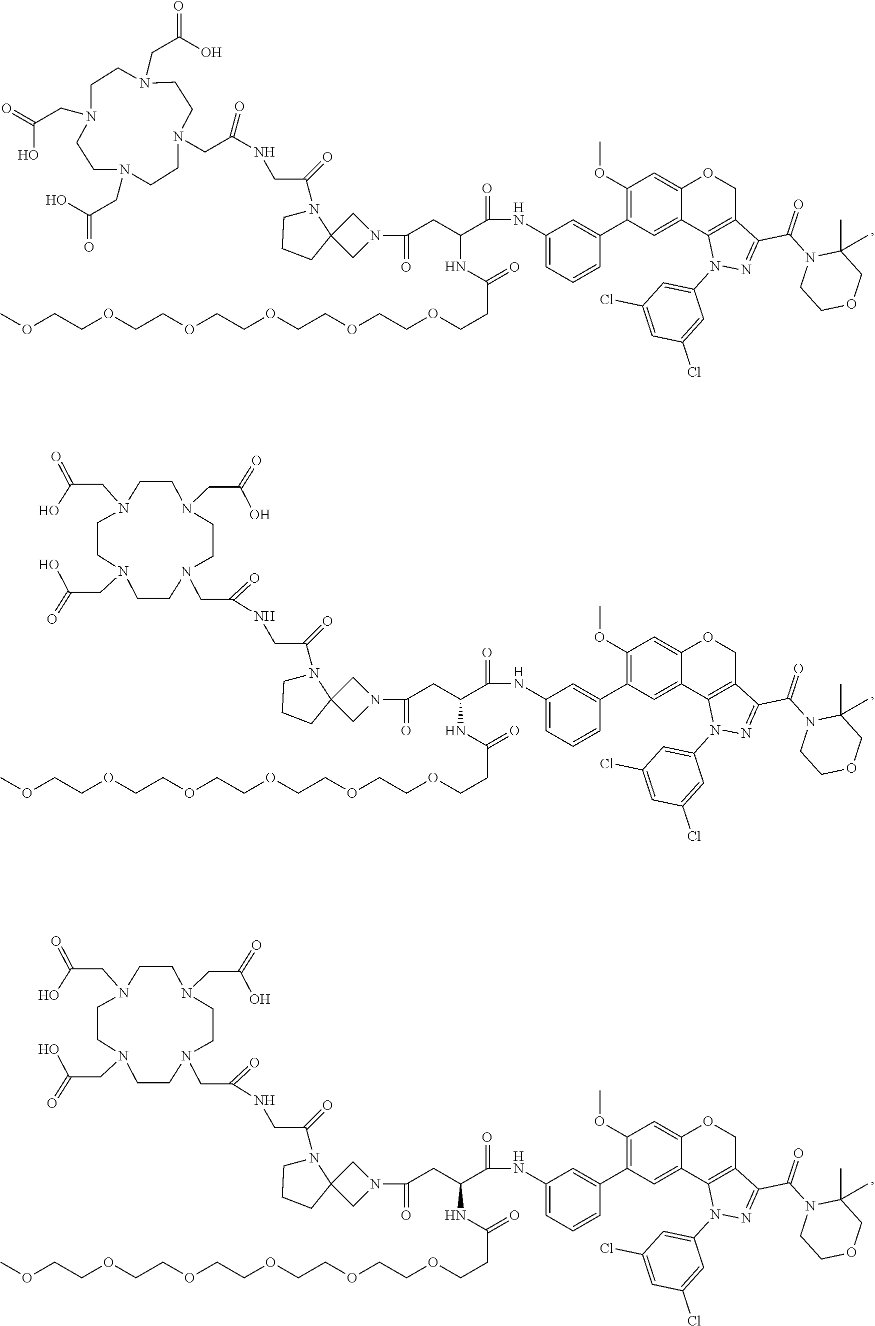

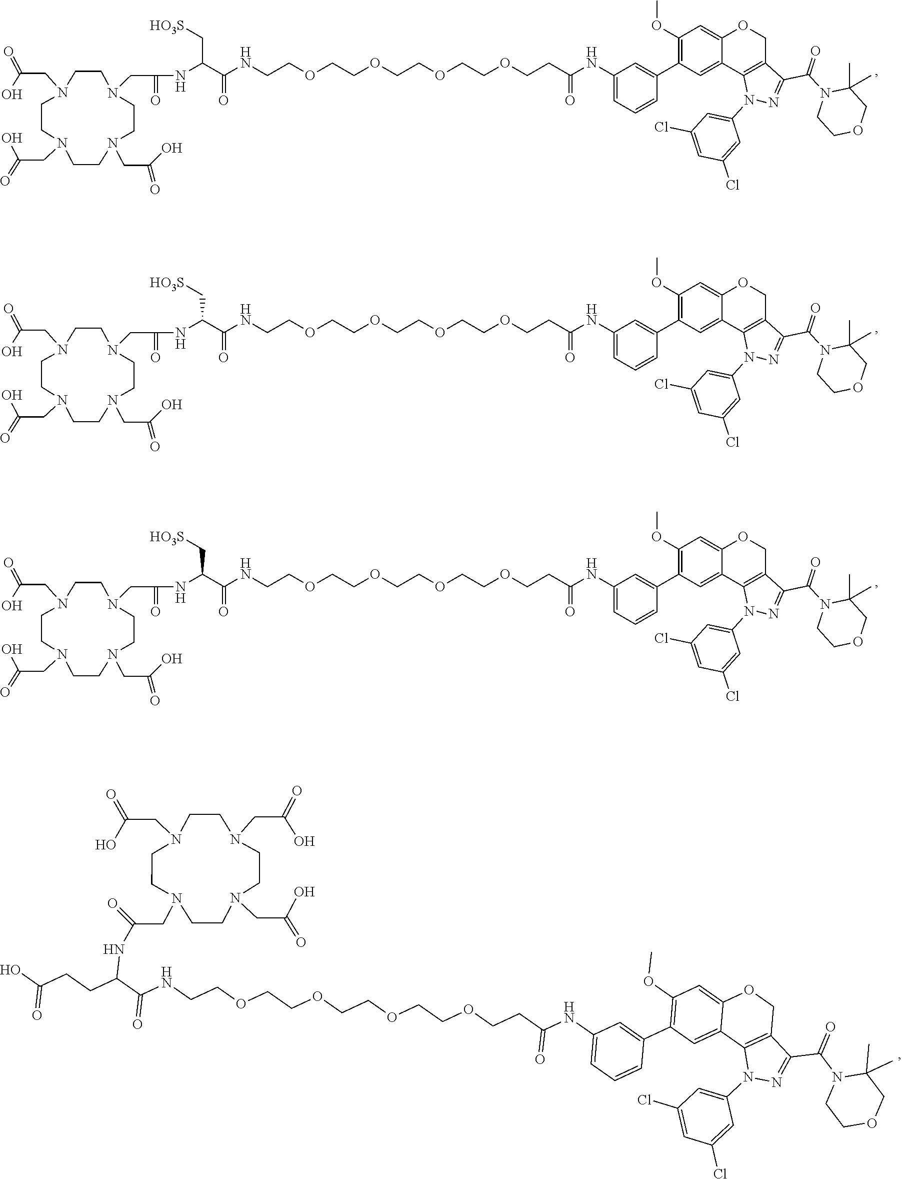

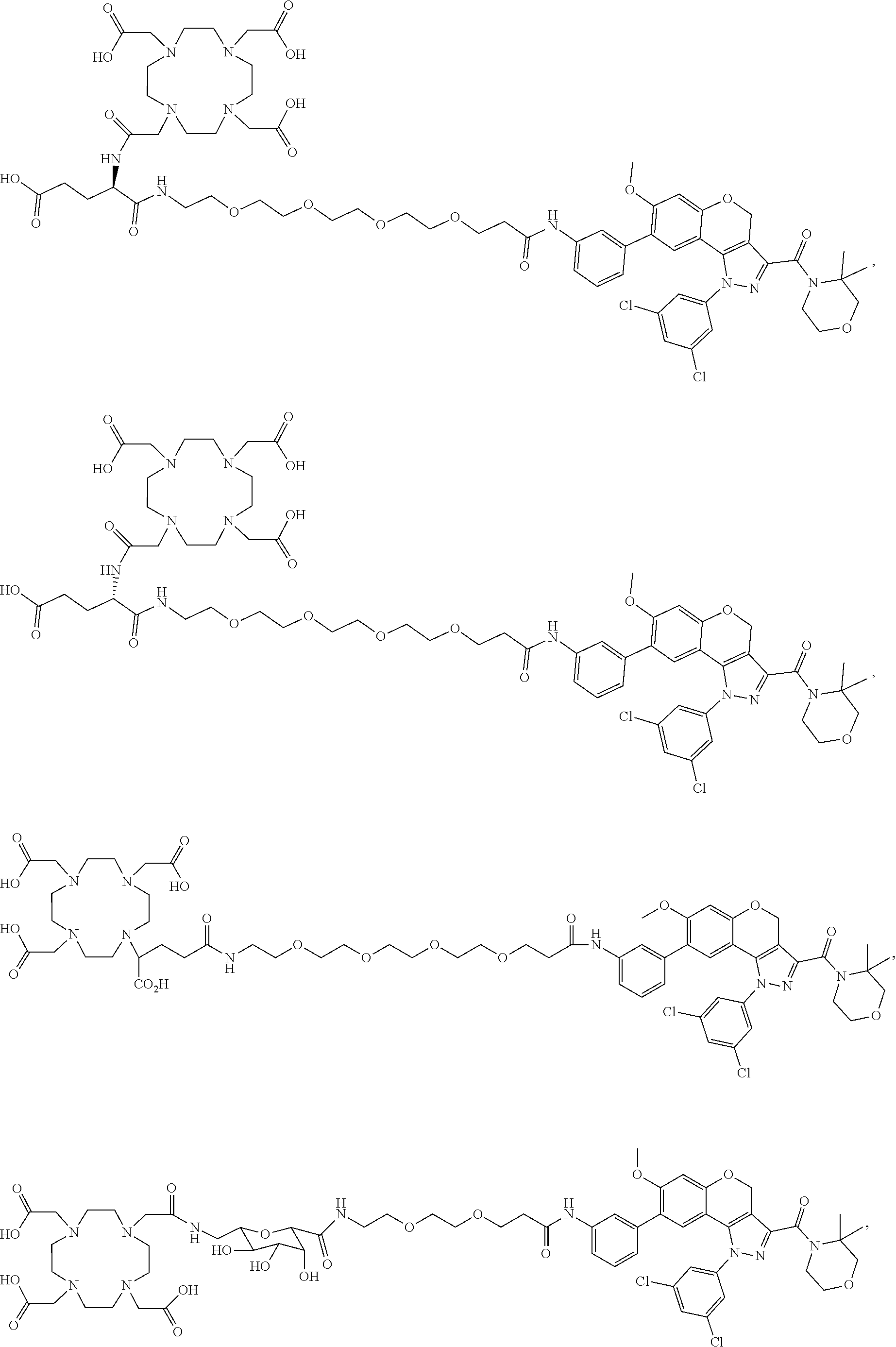

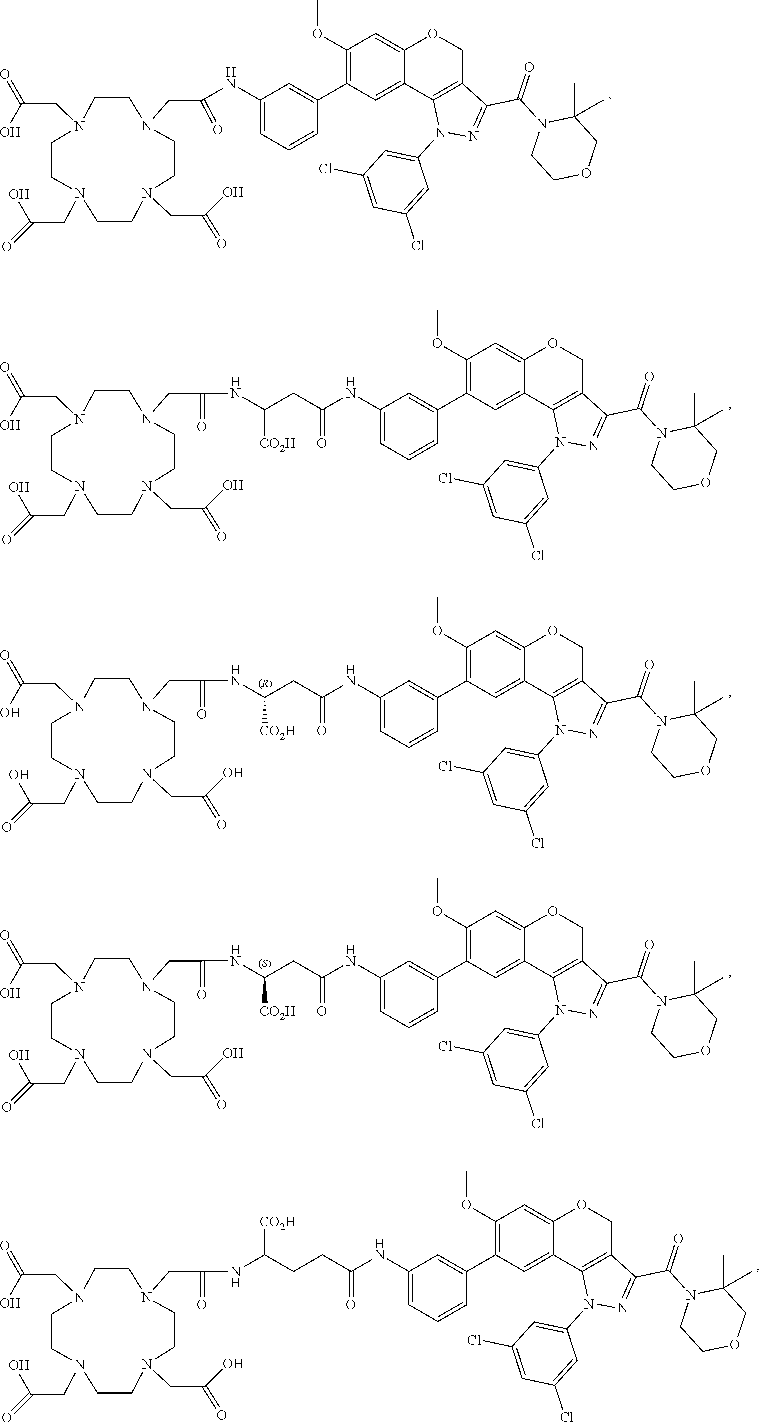

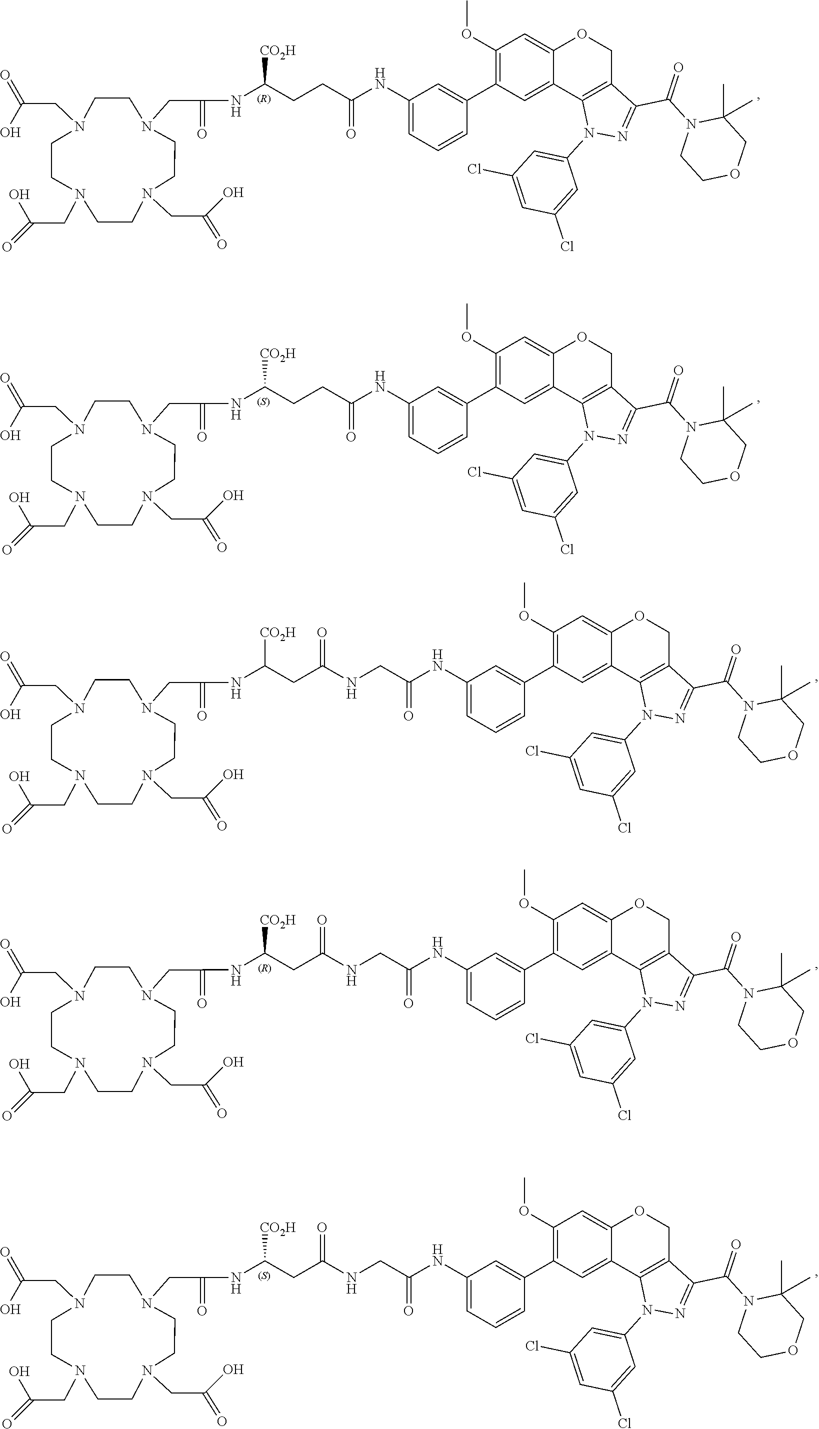

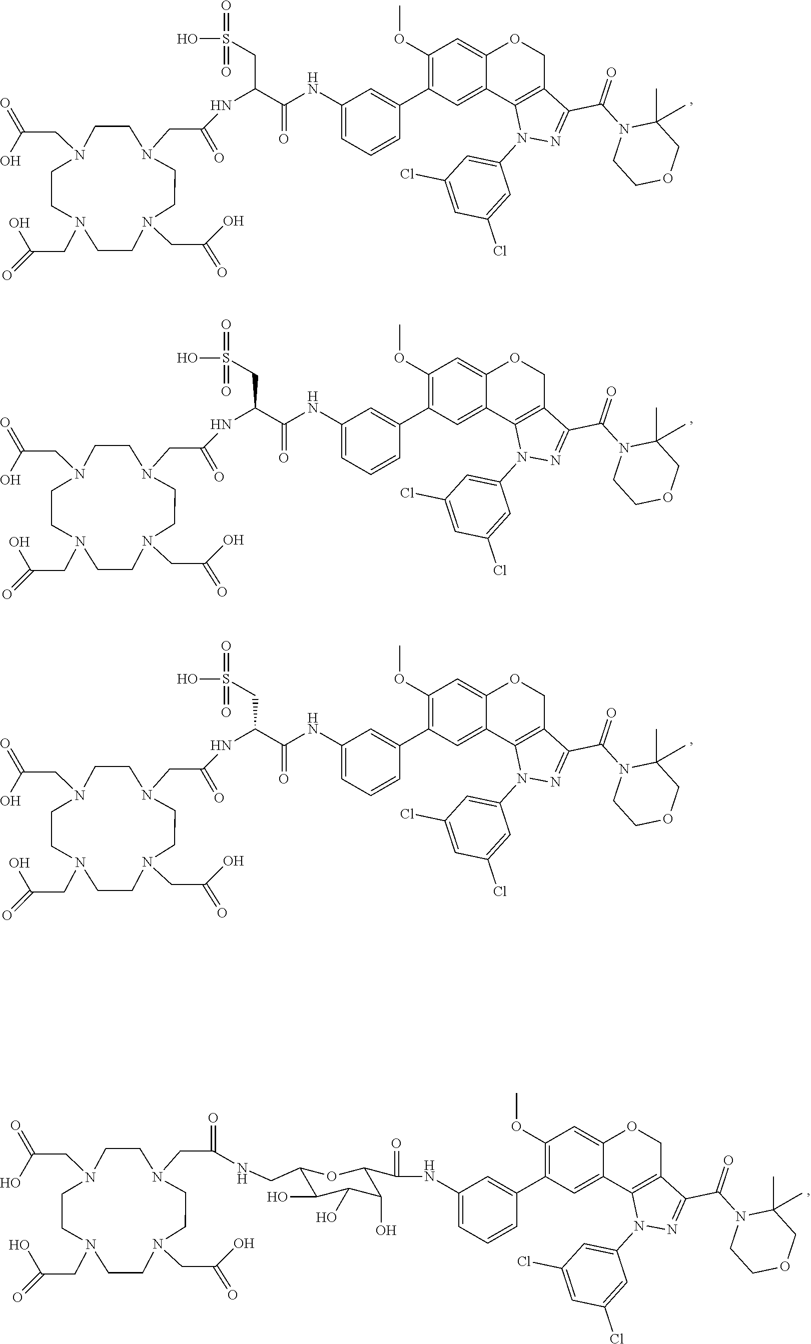

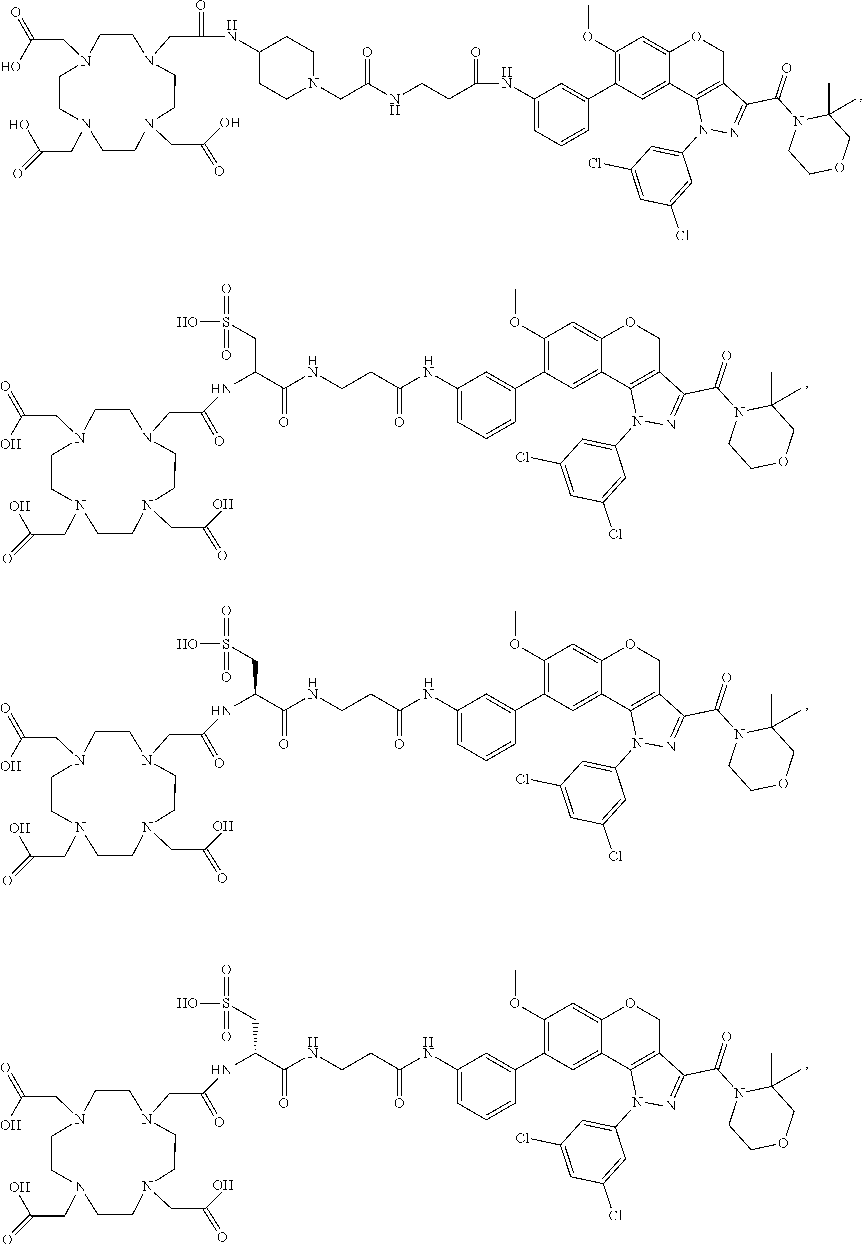

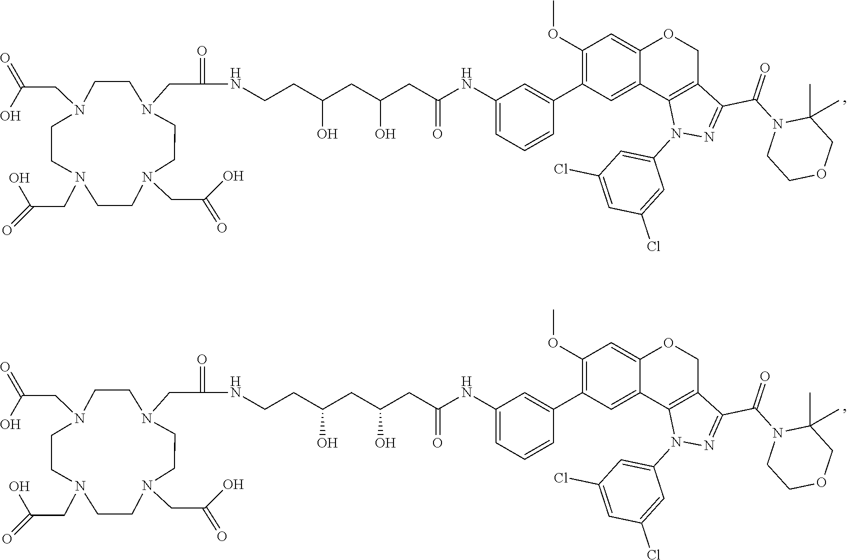 [SMILES: COc1cc2c(cc1-c1cccc(NC(=O)CC(O)CC(O)CCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2 ; COc1cc2c(cc1-c1cccc(NC(=O)C[C@H](O)C[C@H](O)CCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2]

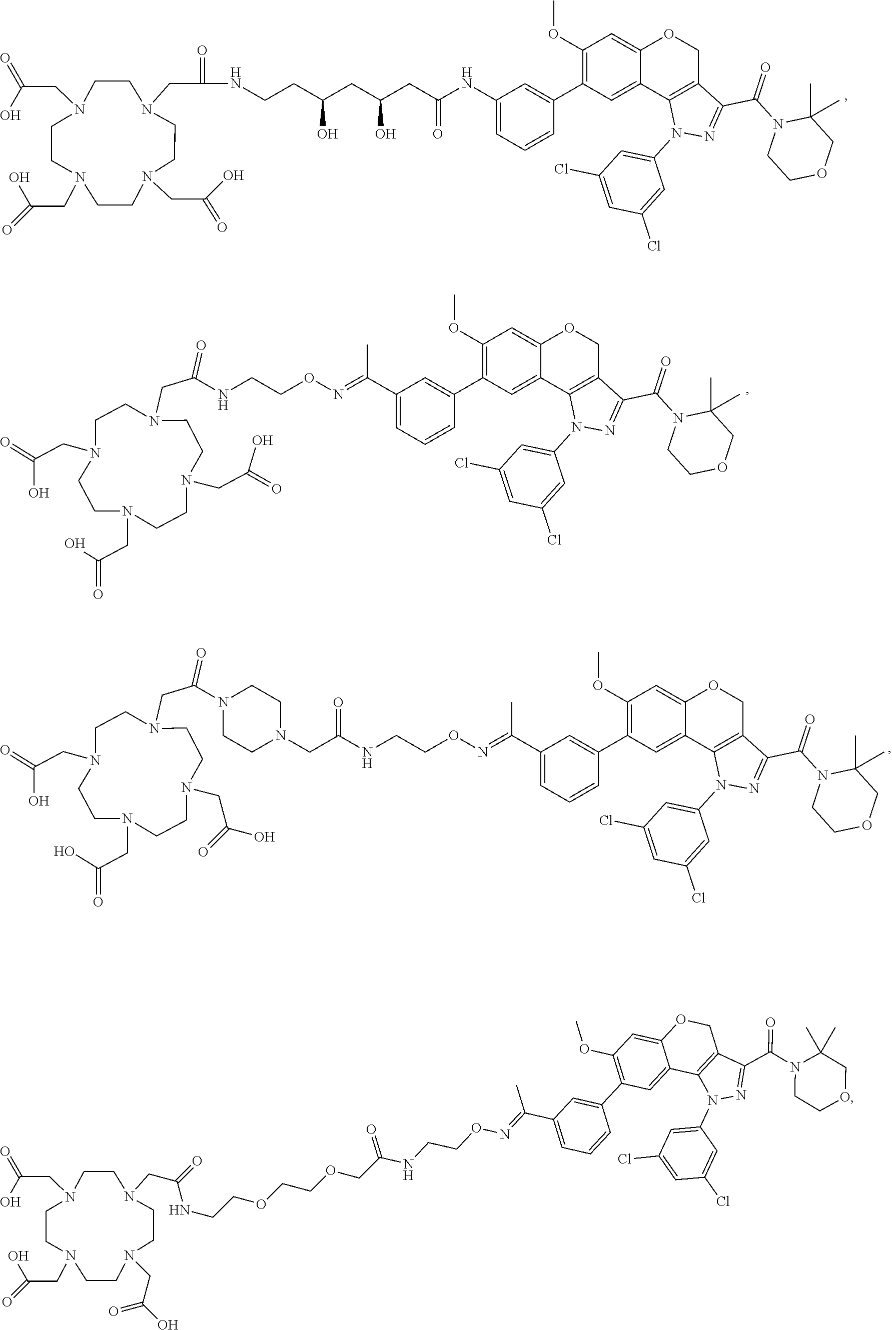

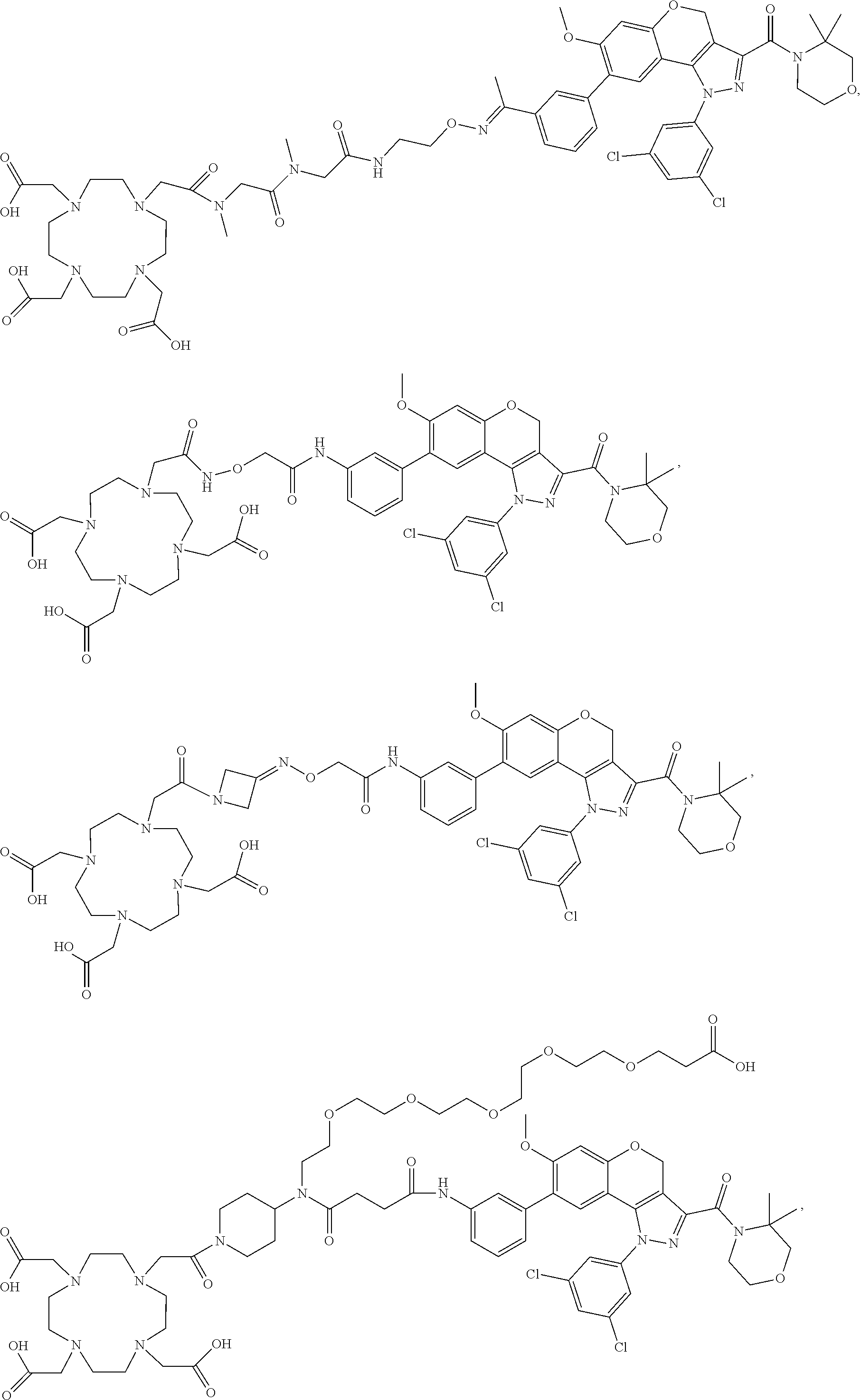

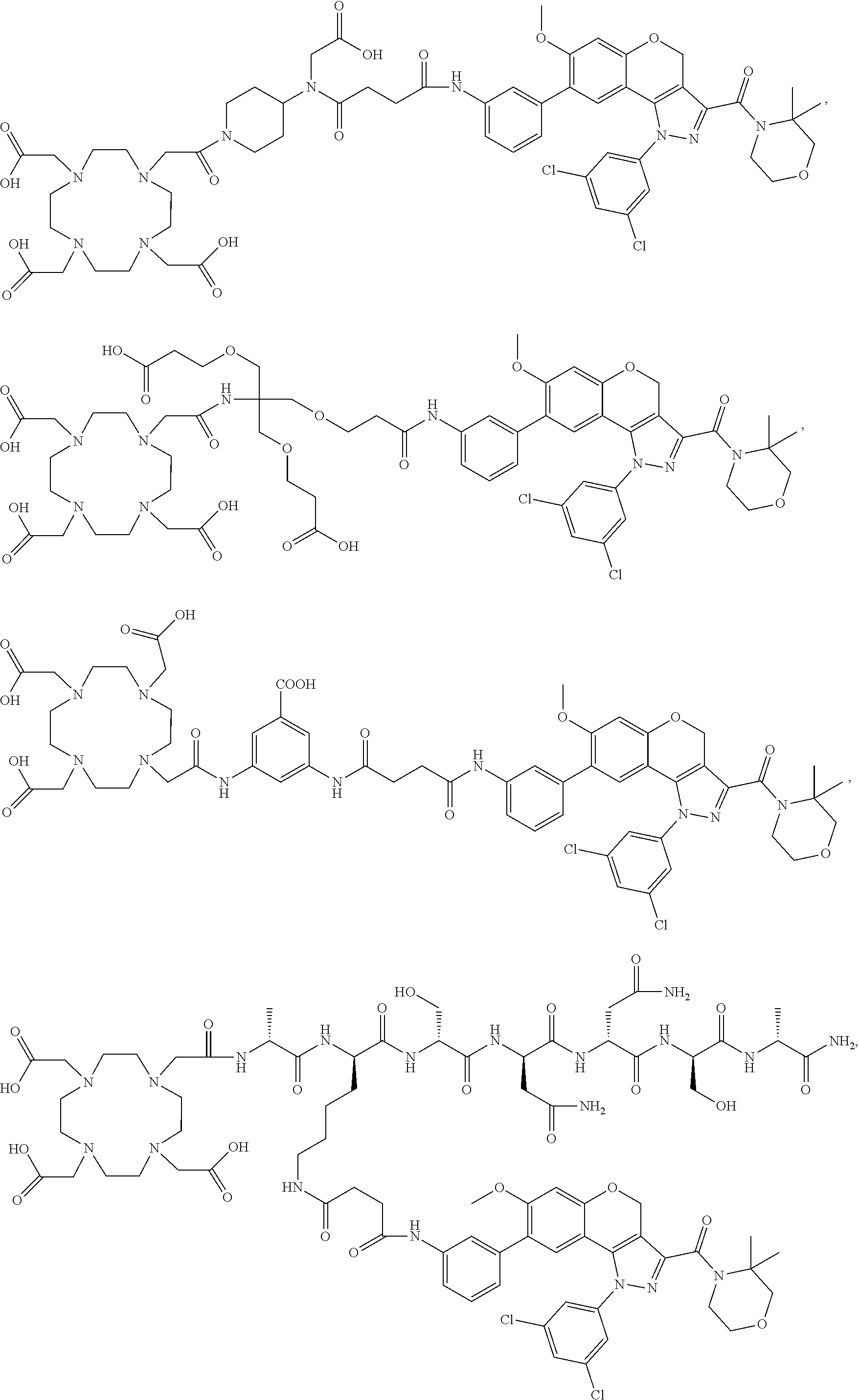

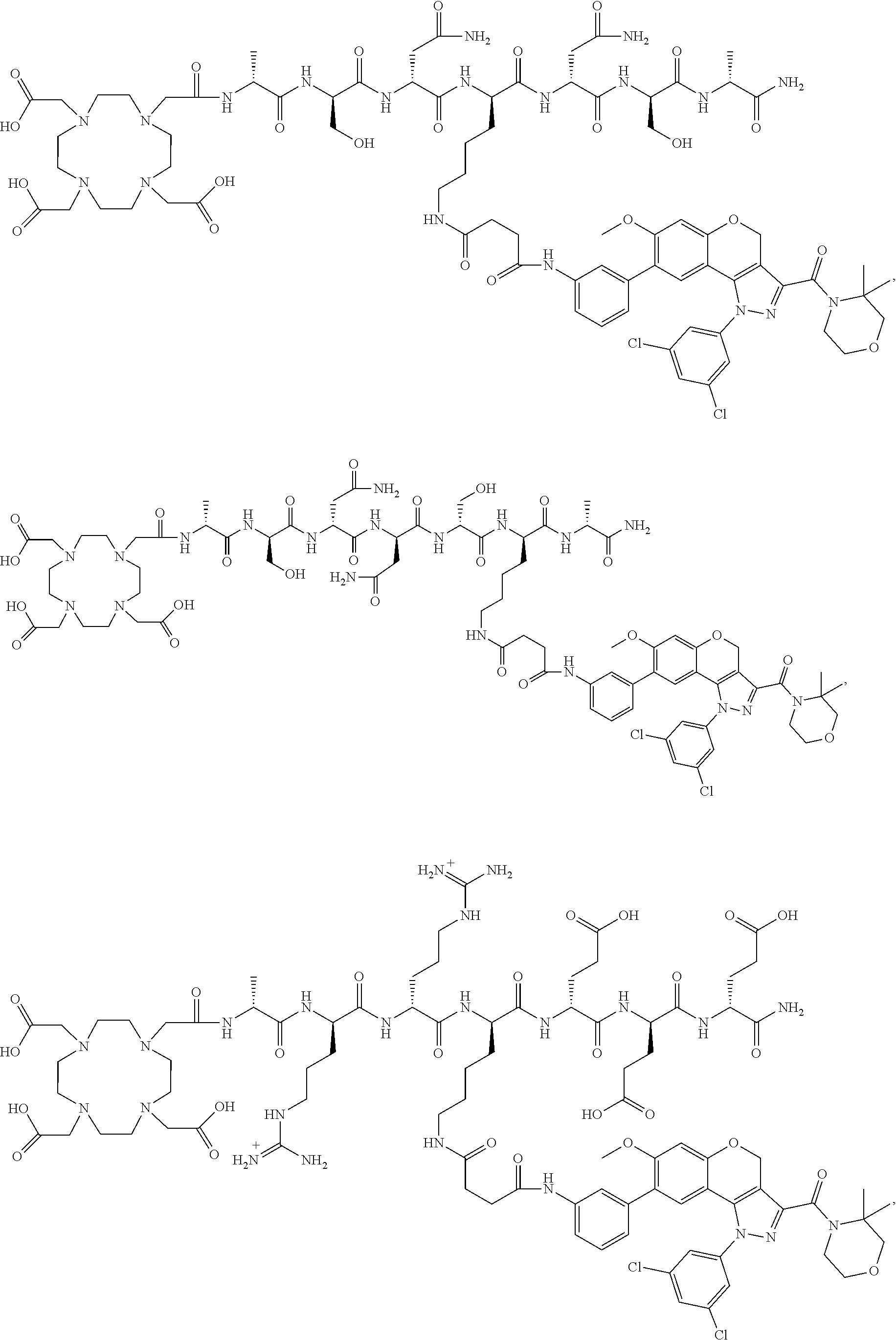

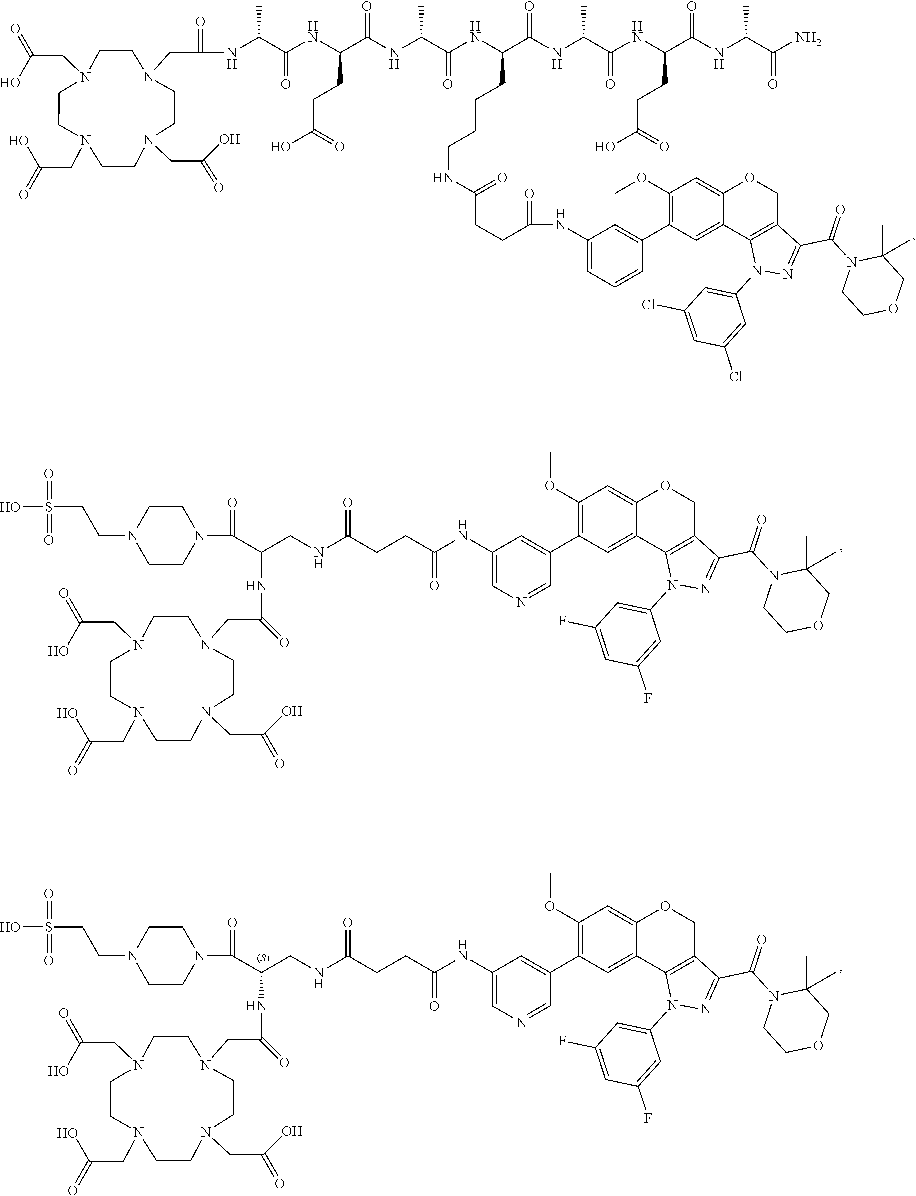

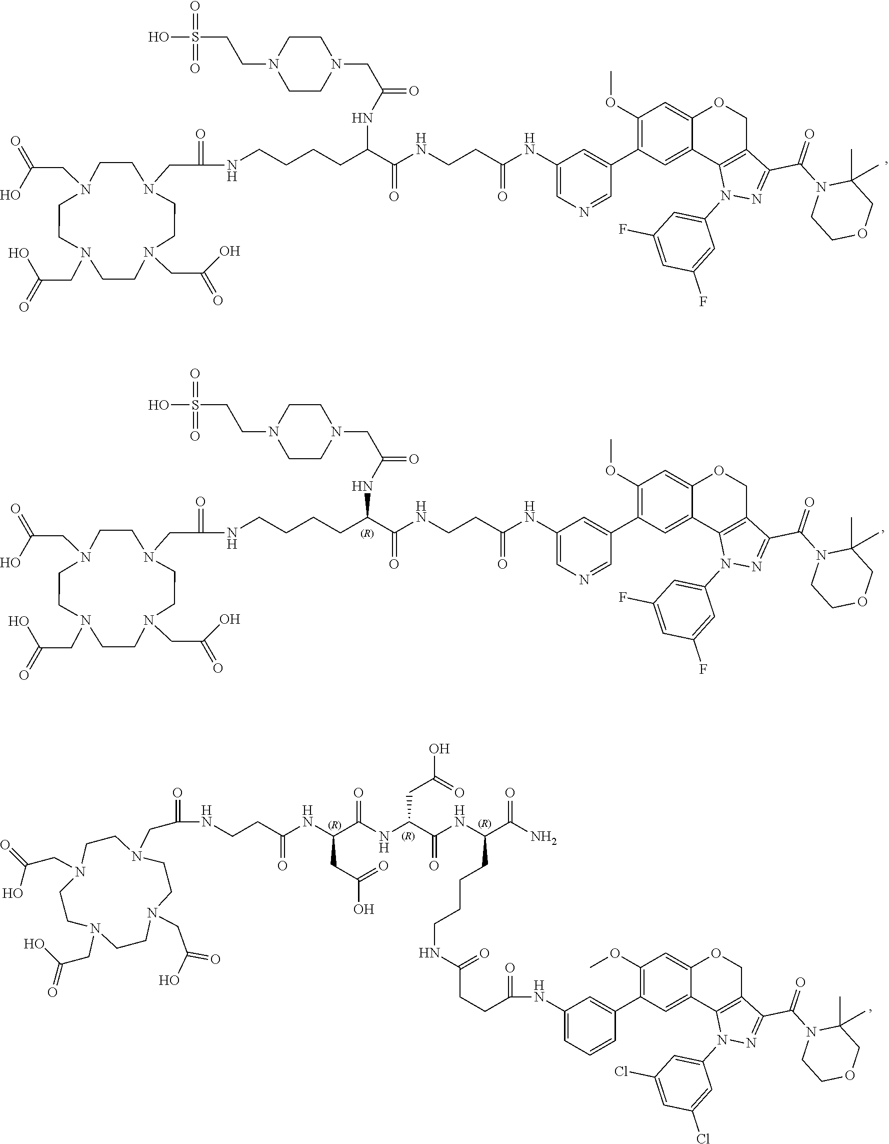

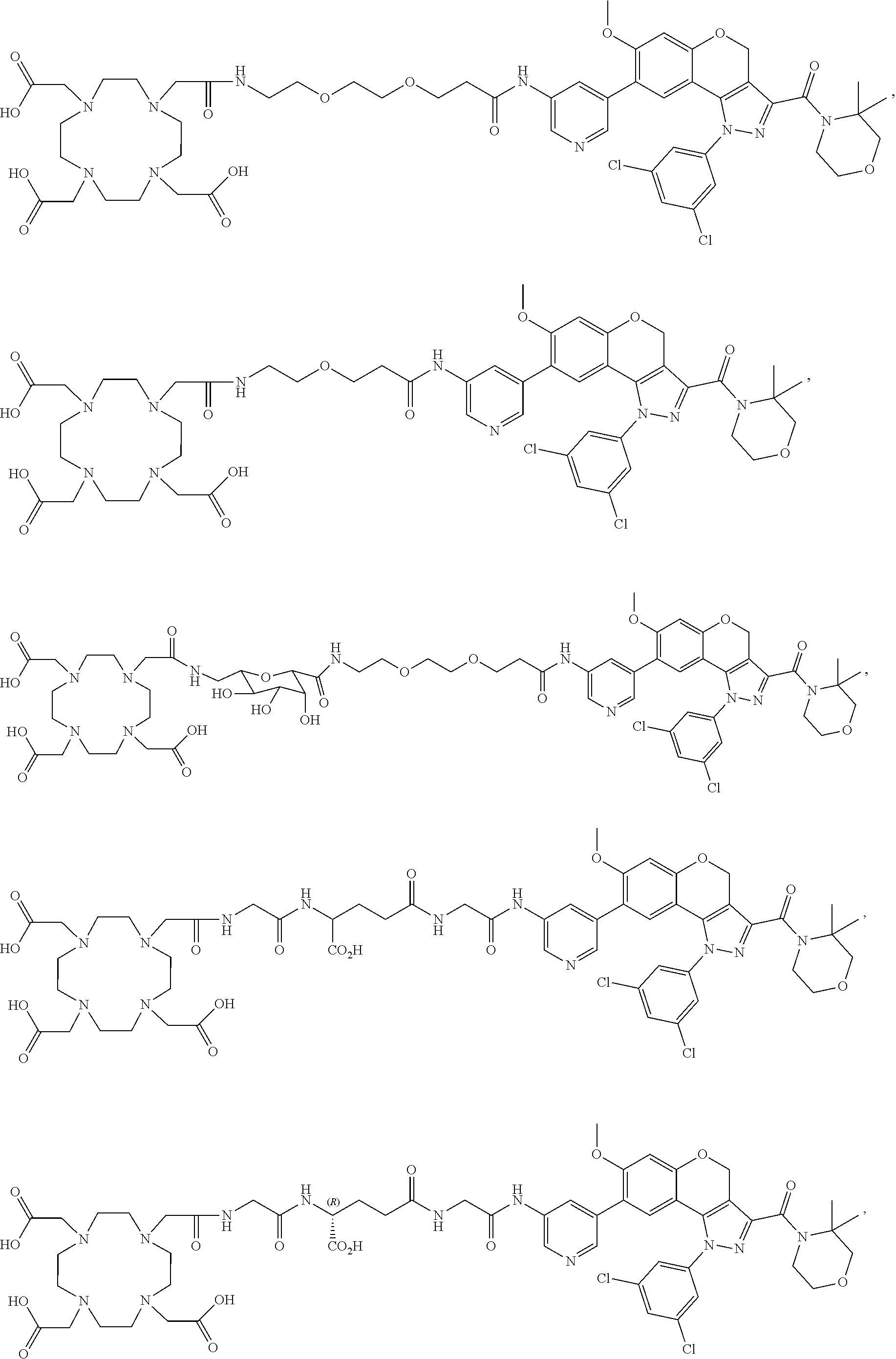

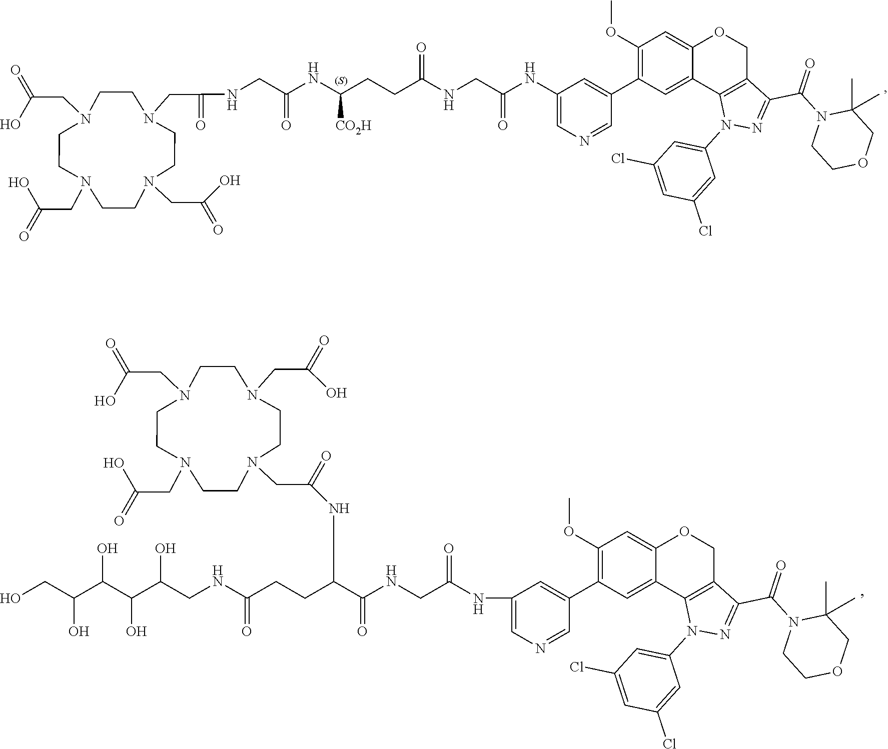

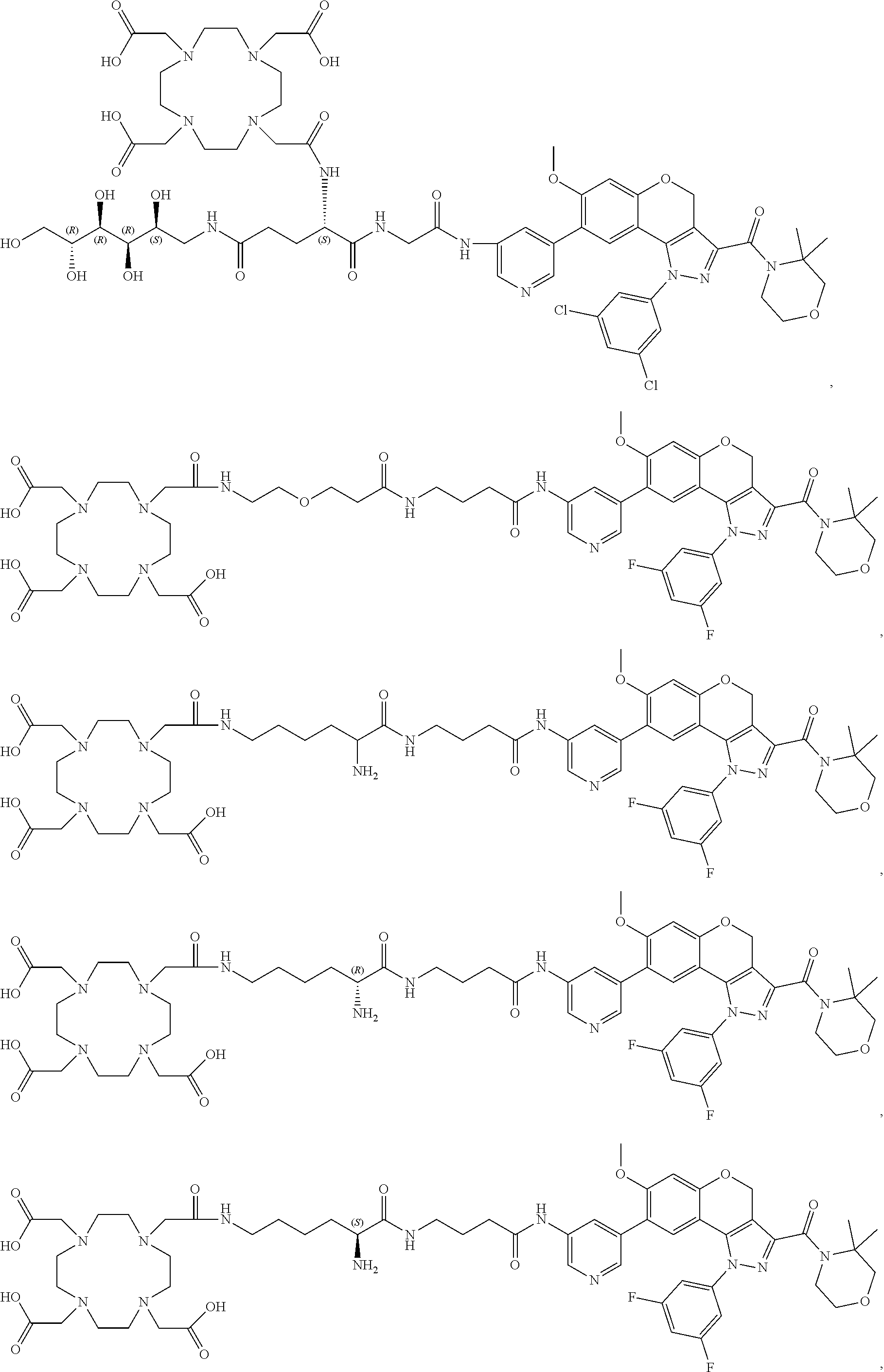

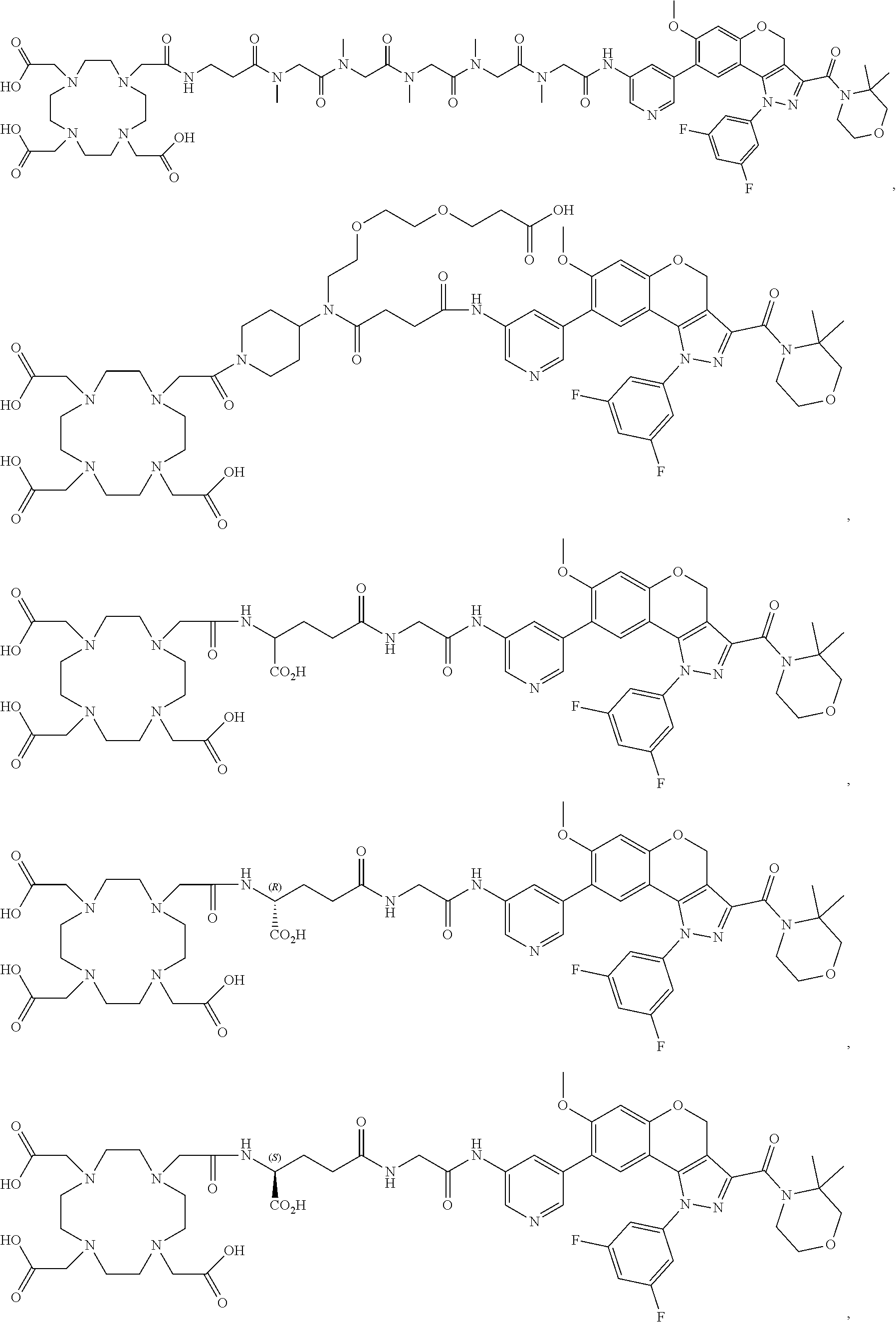

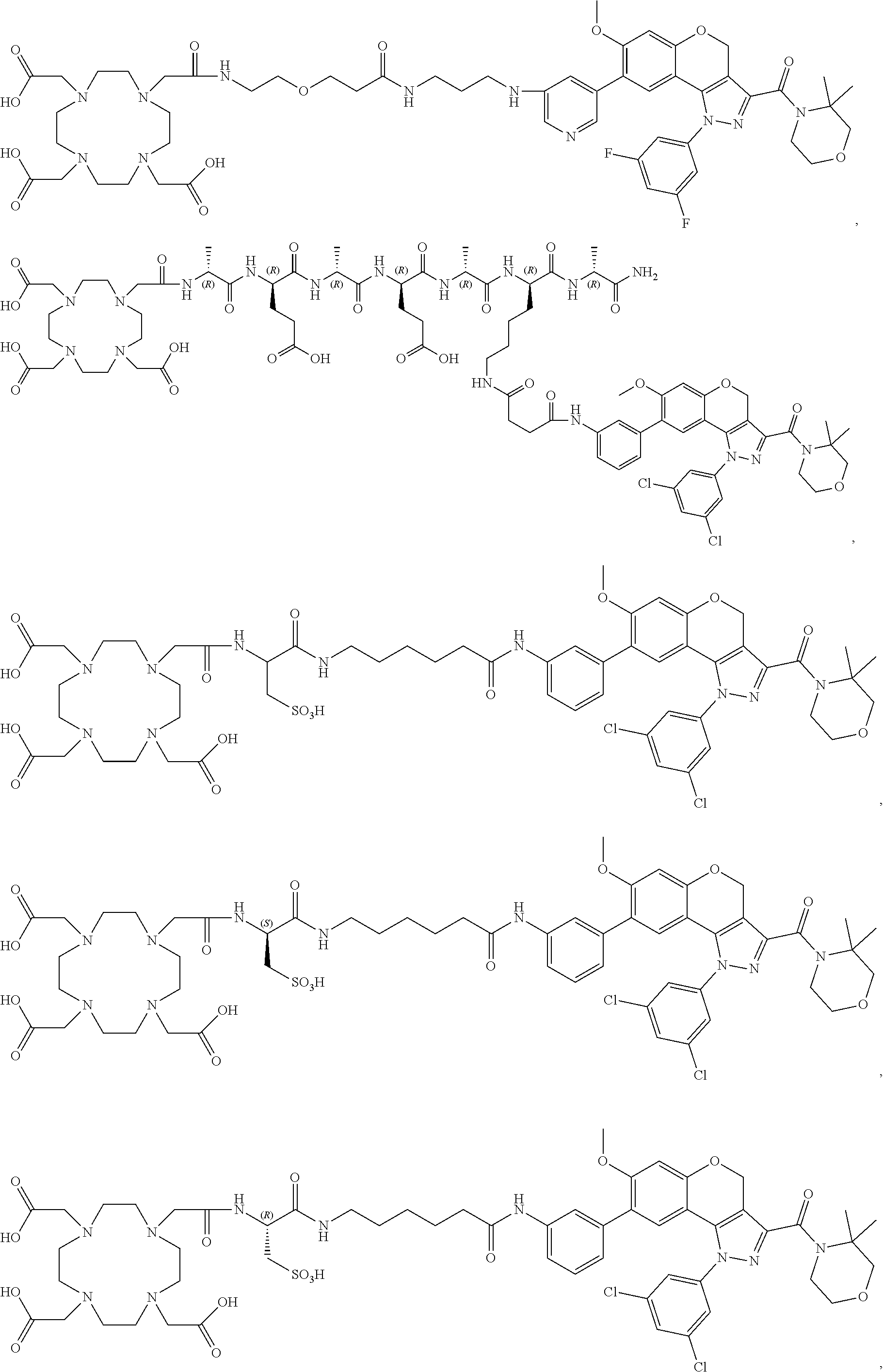

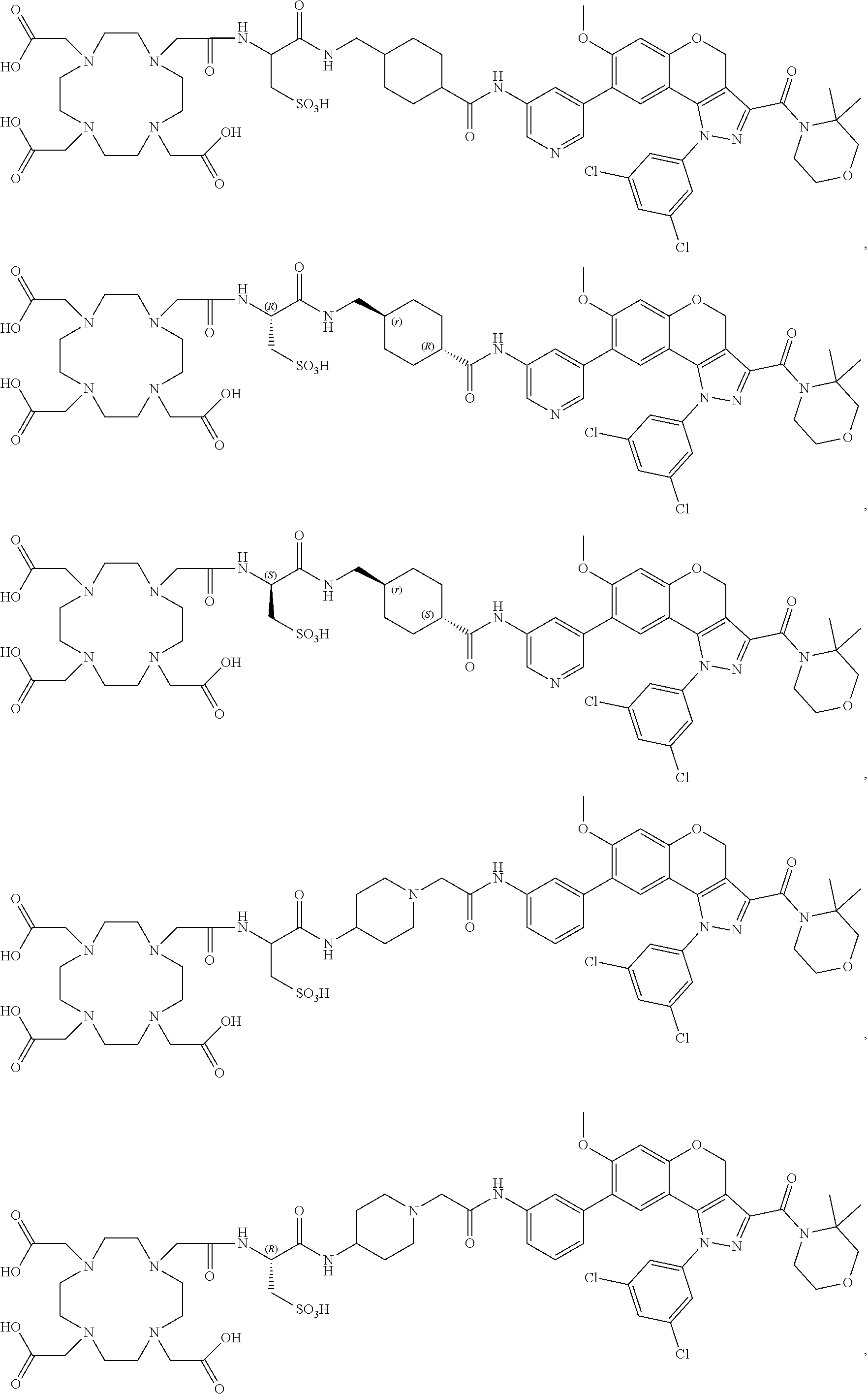

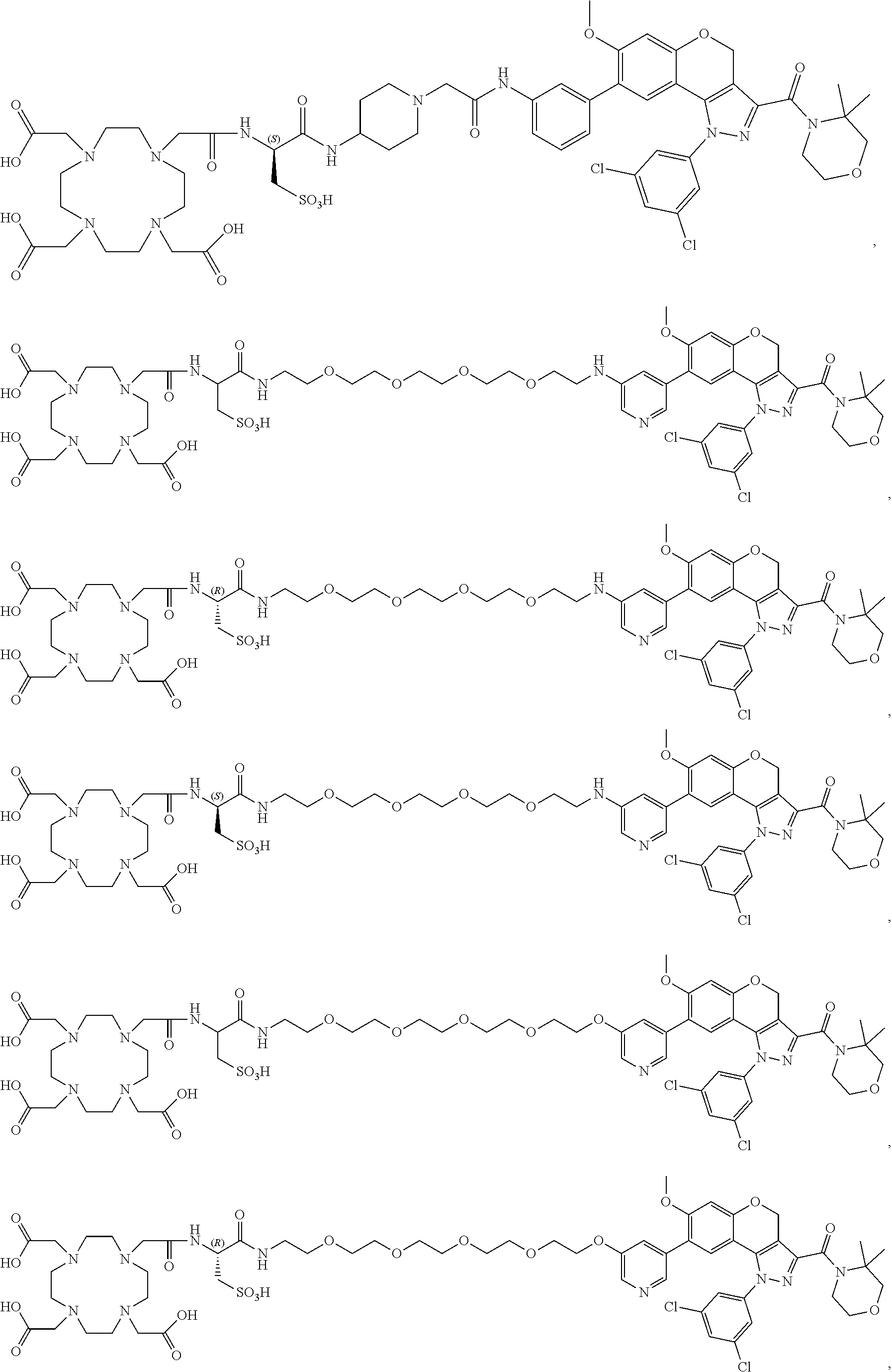

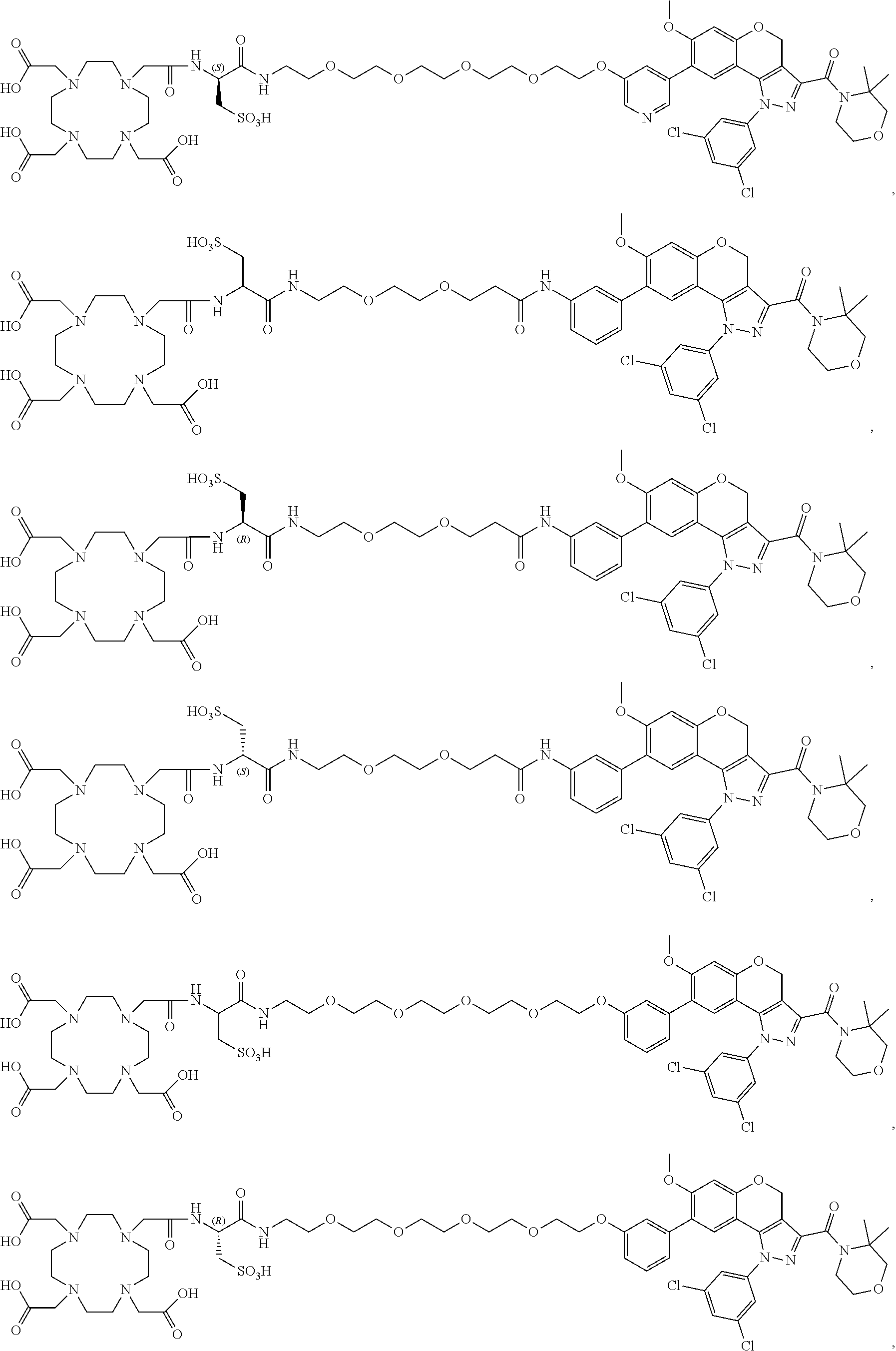

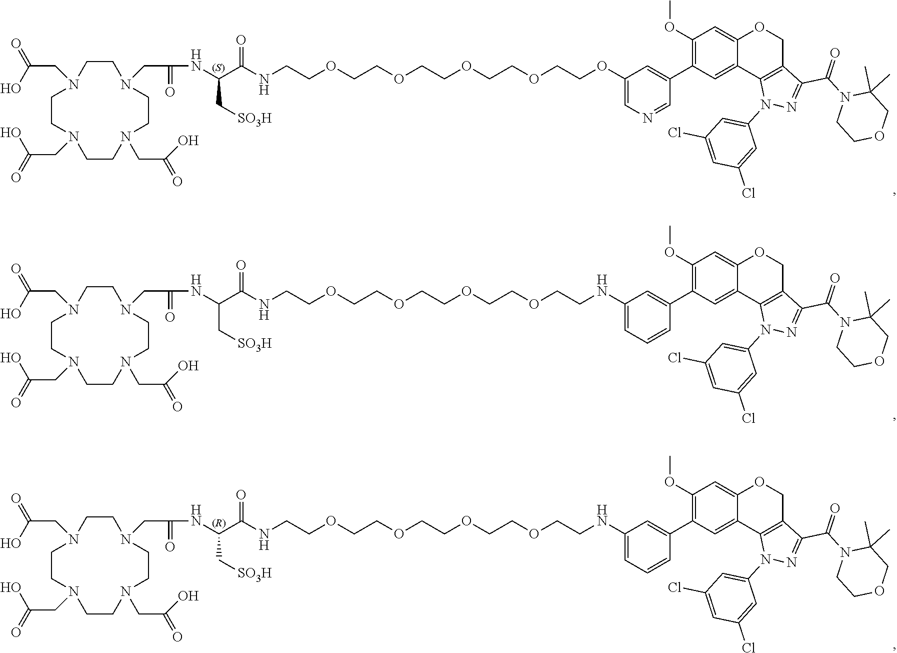

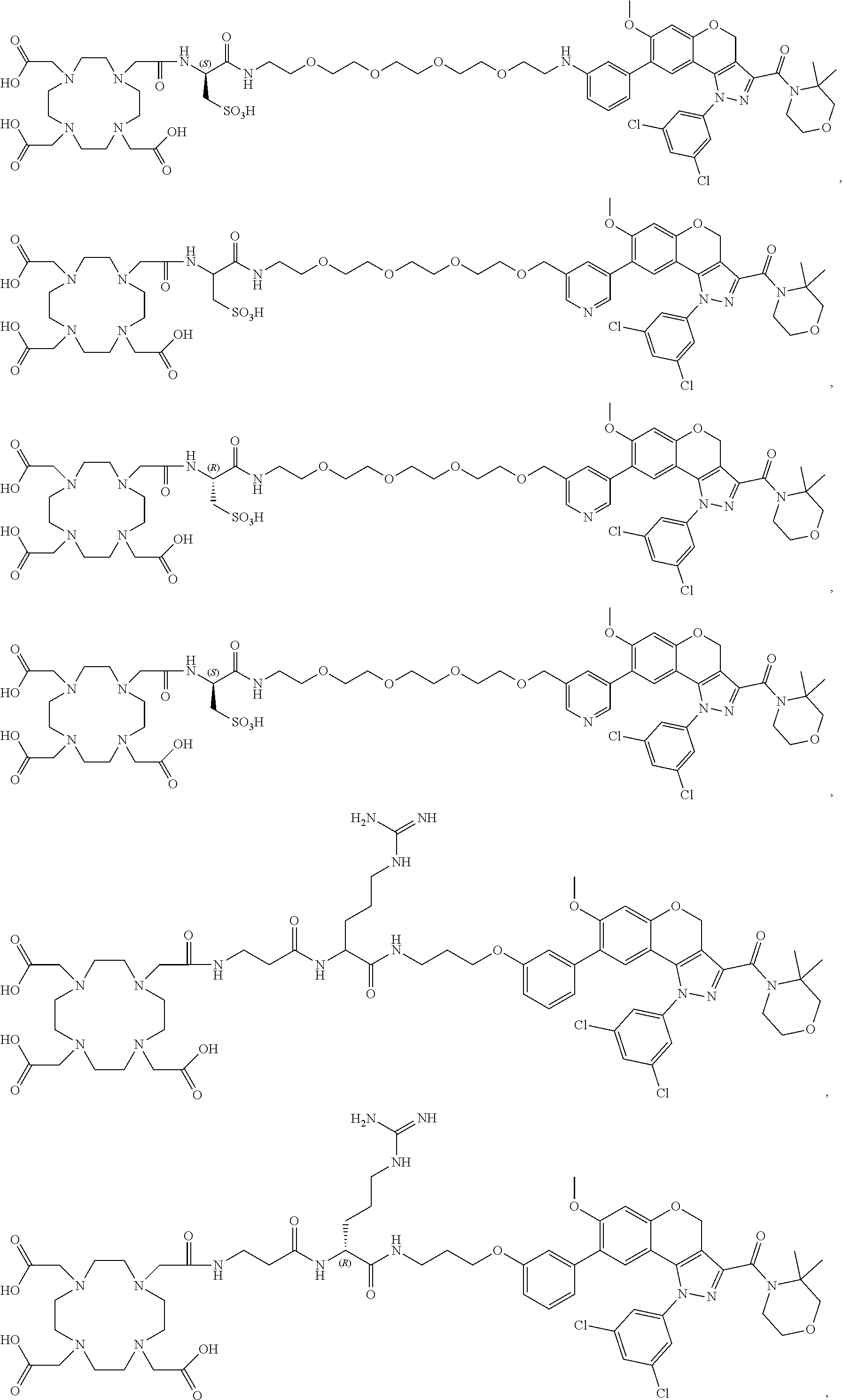

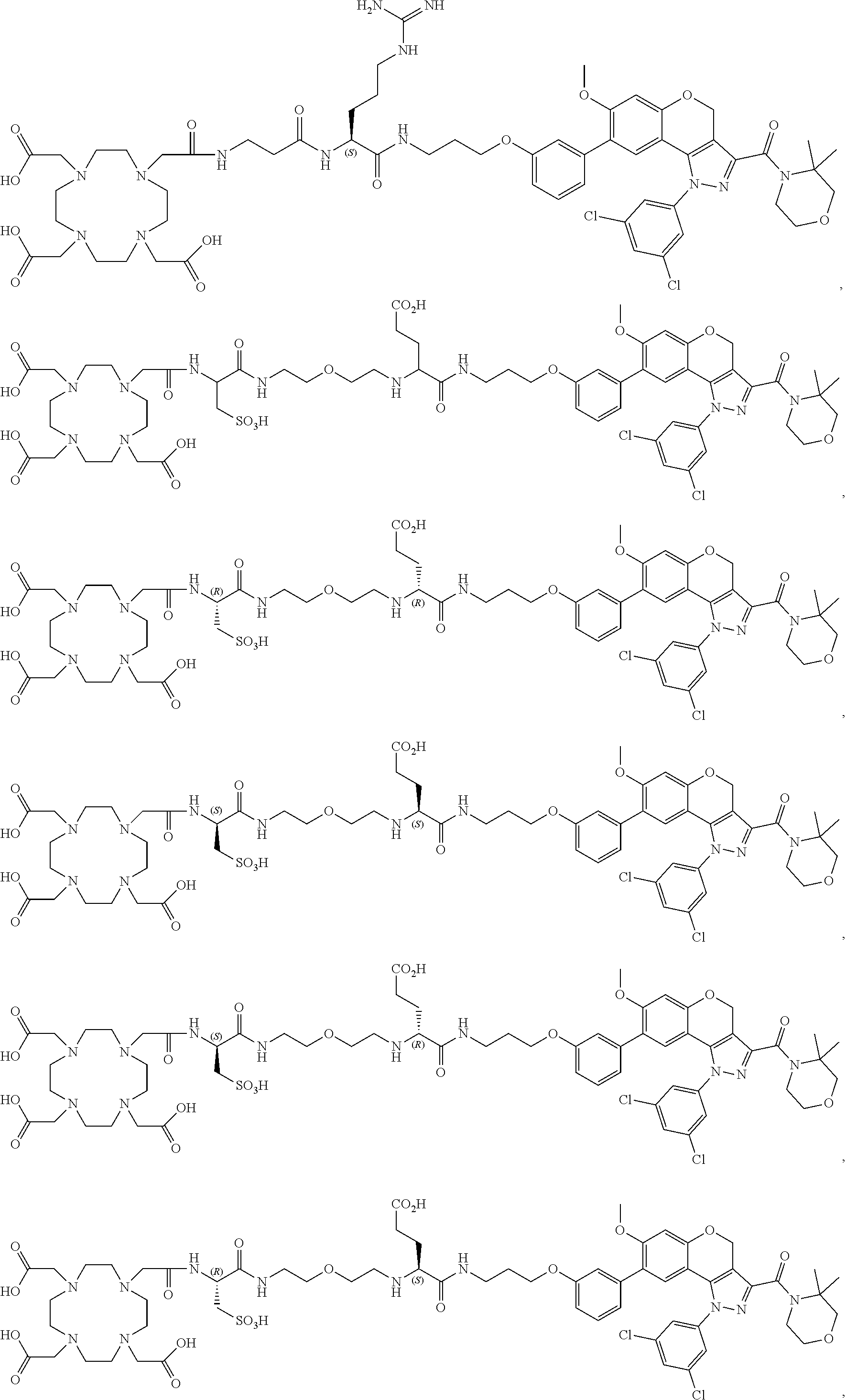

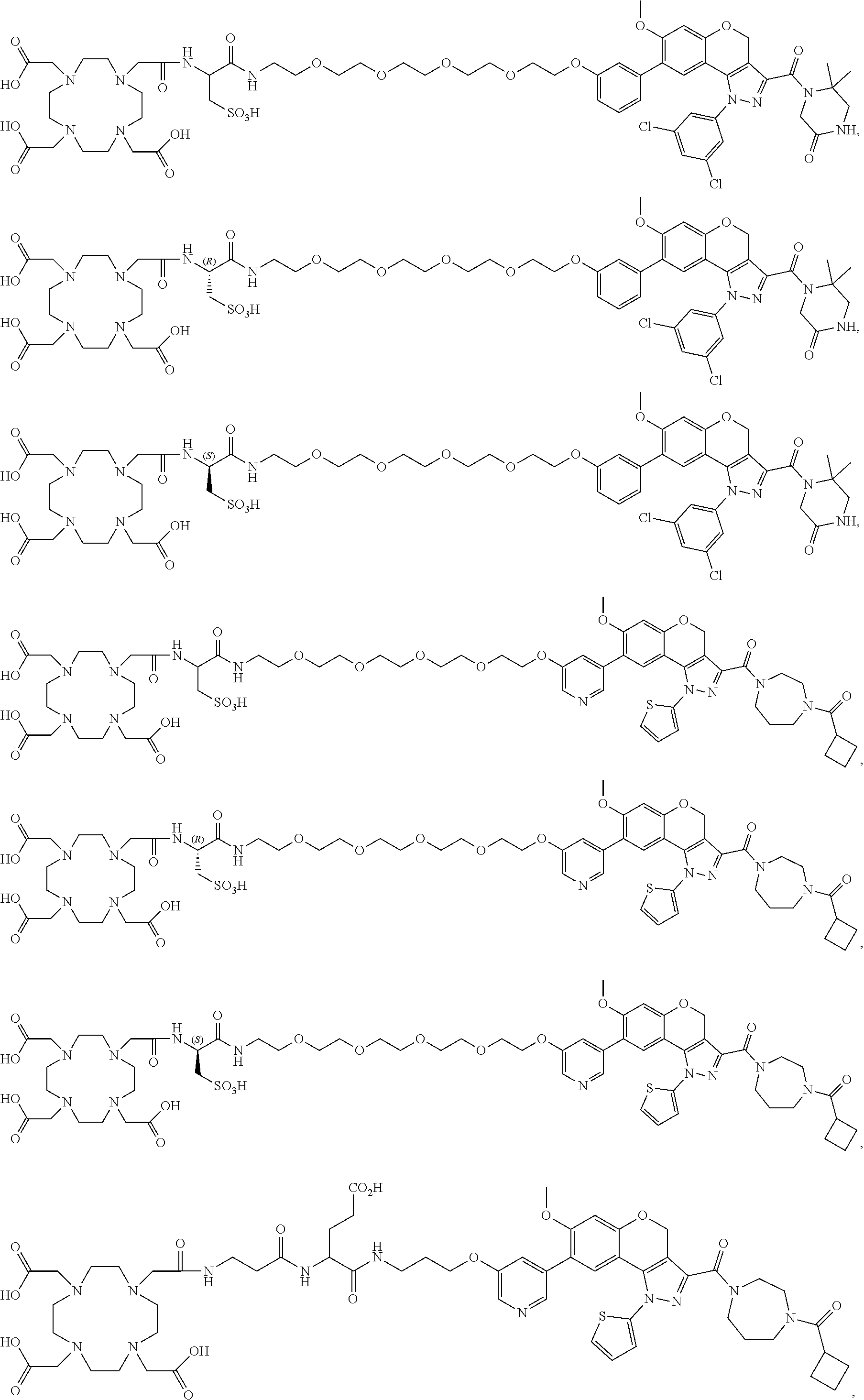

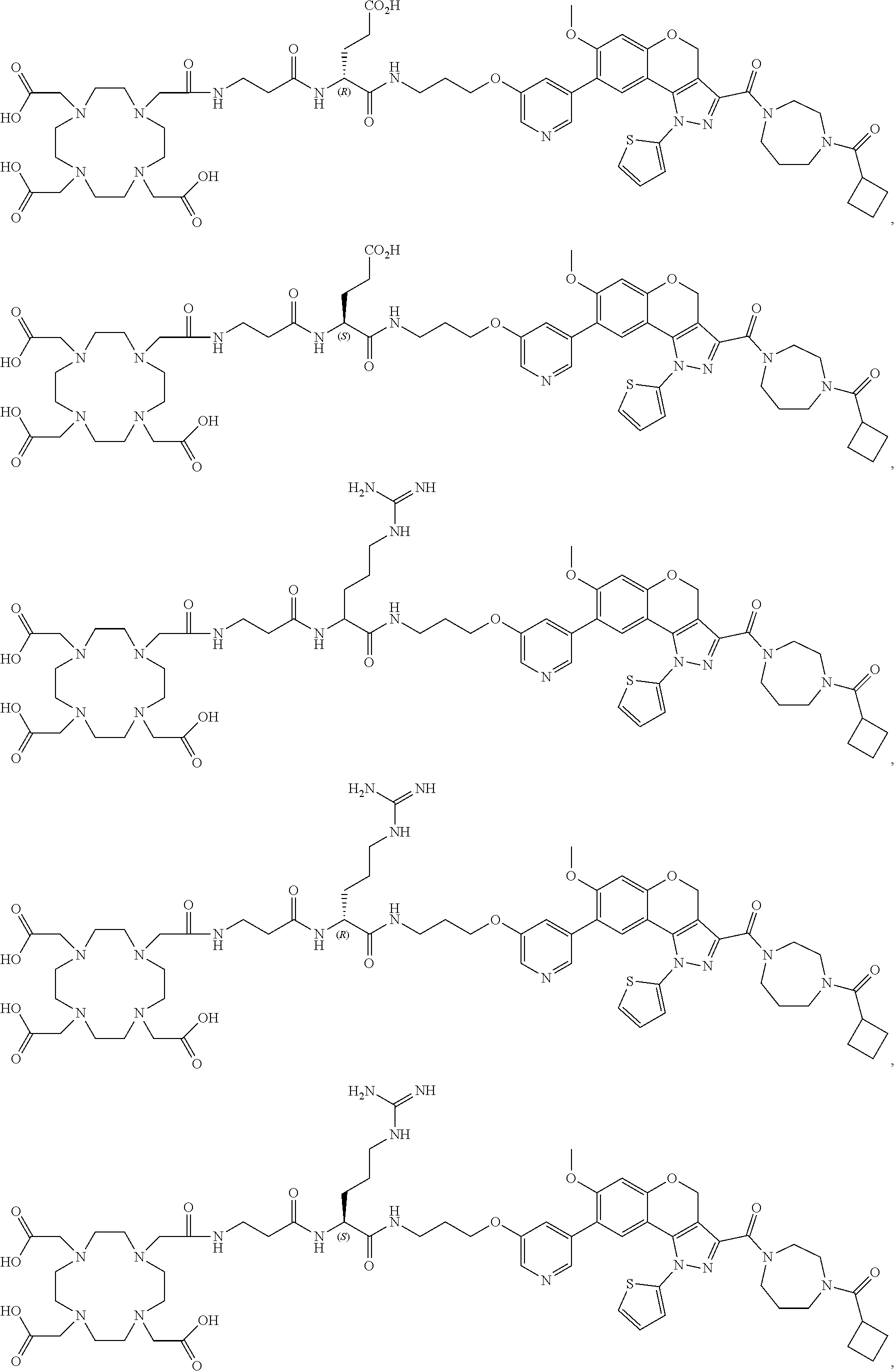

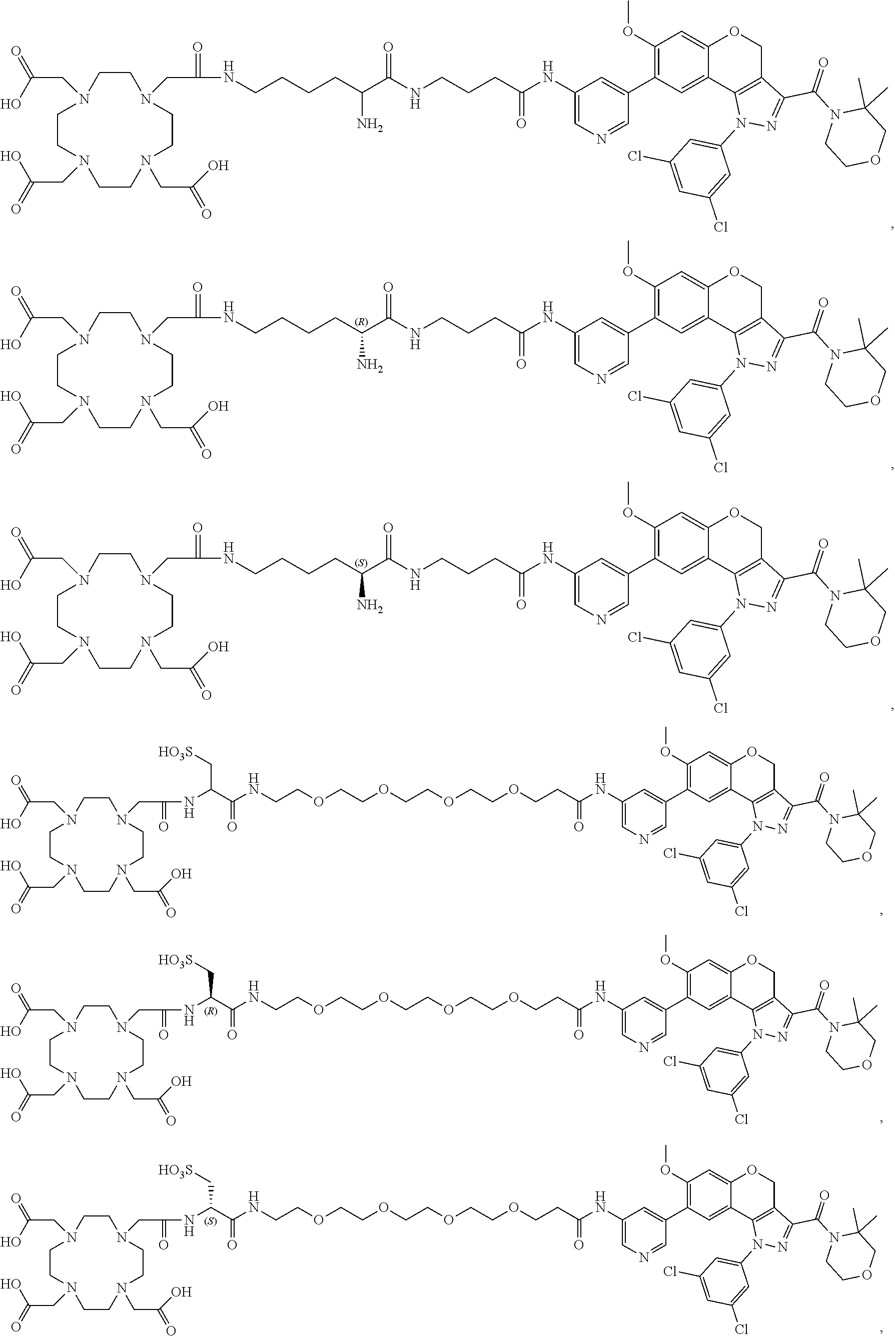

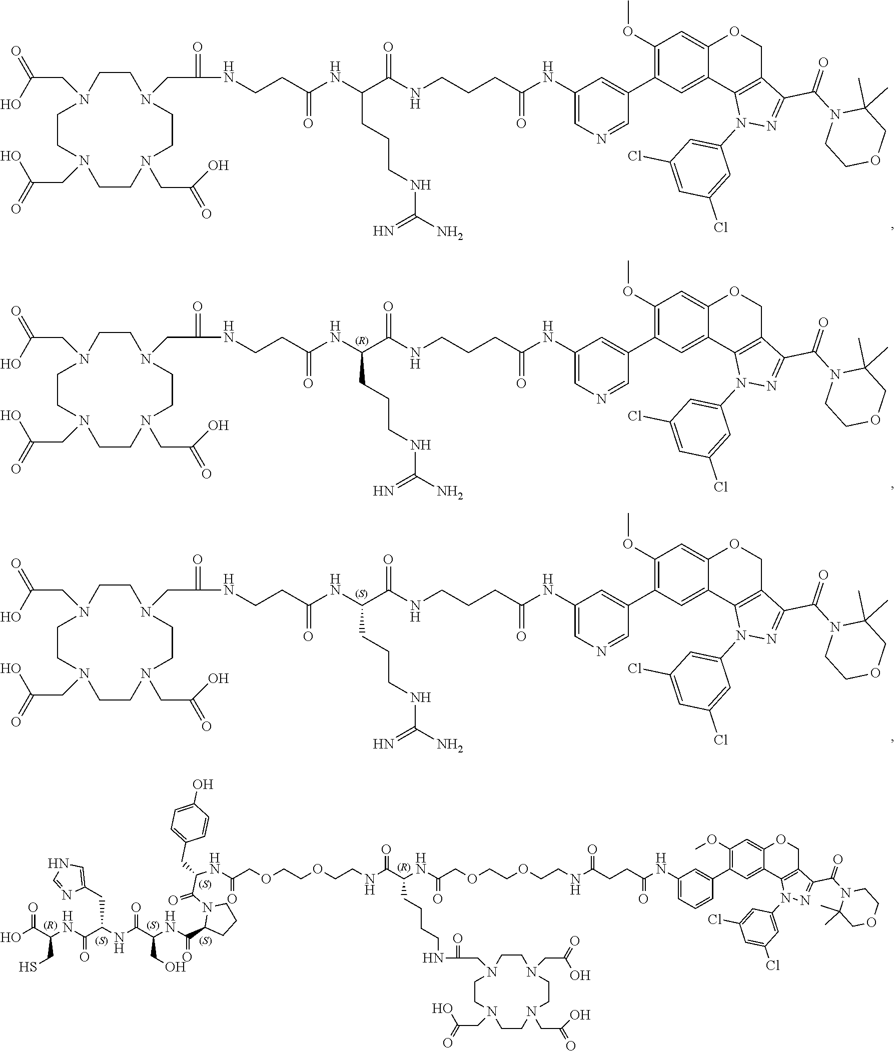

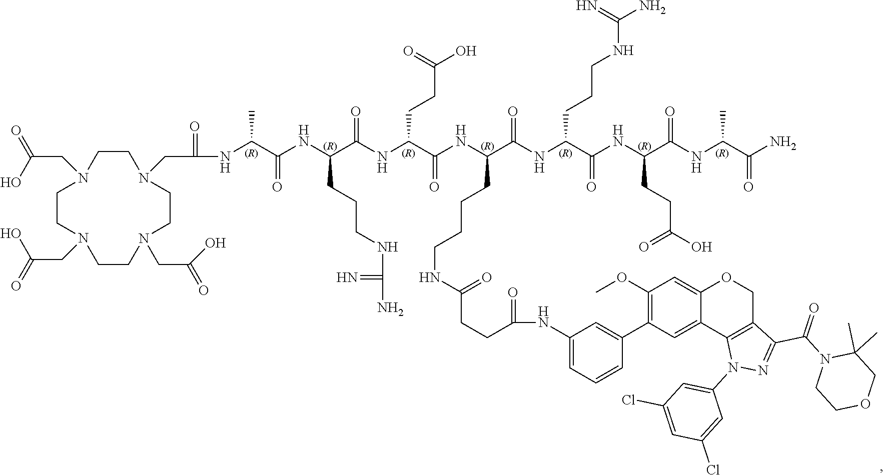

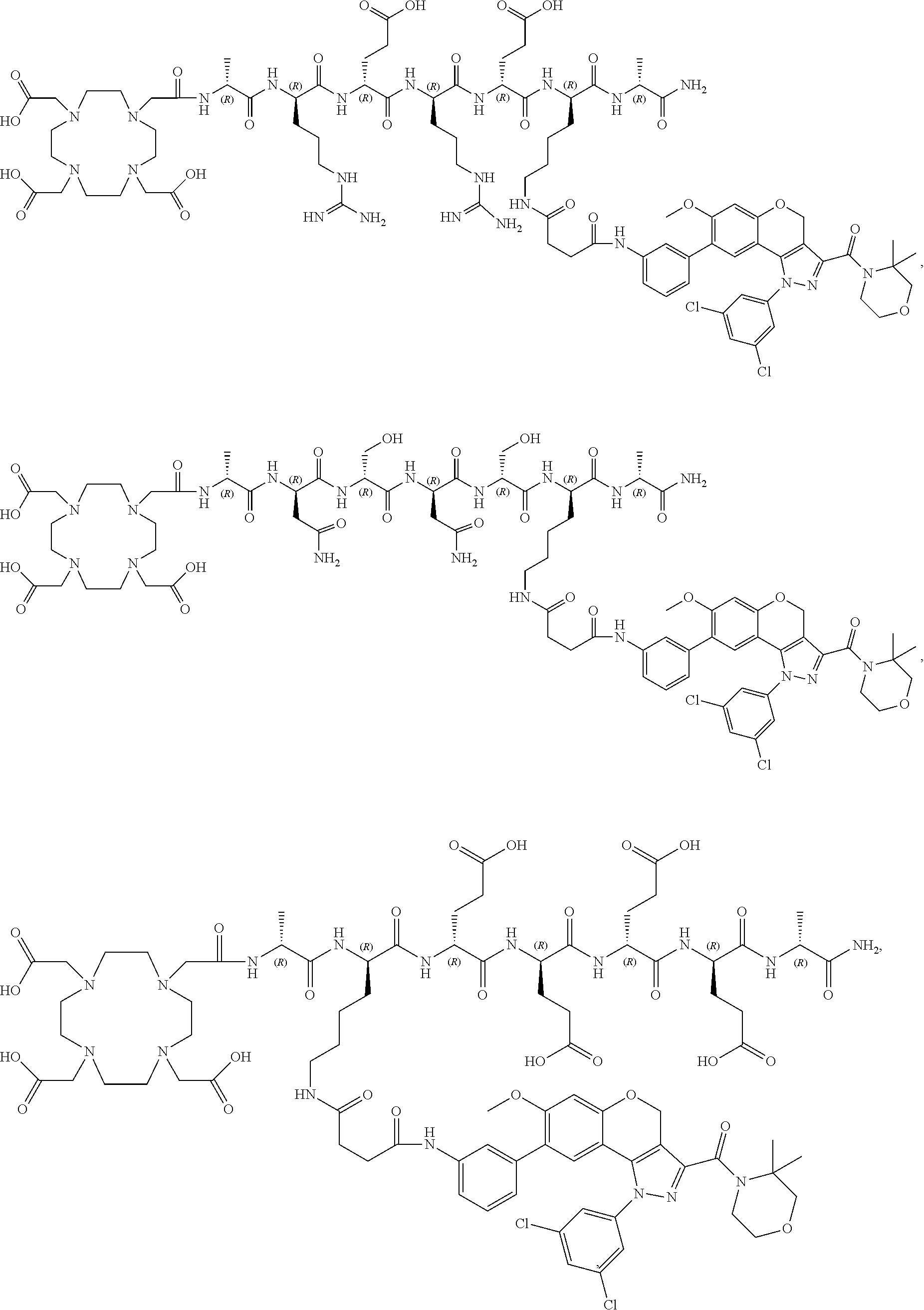

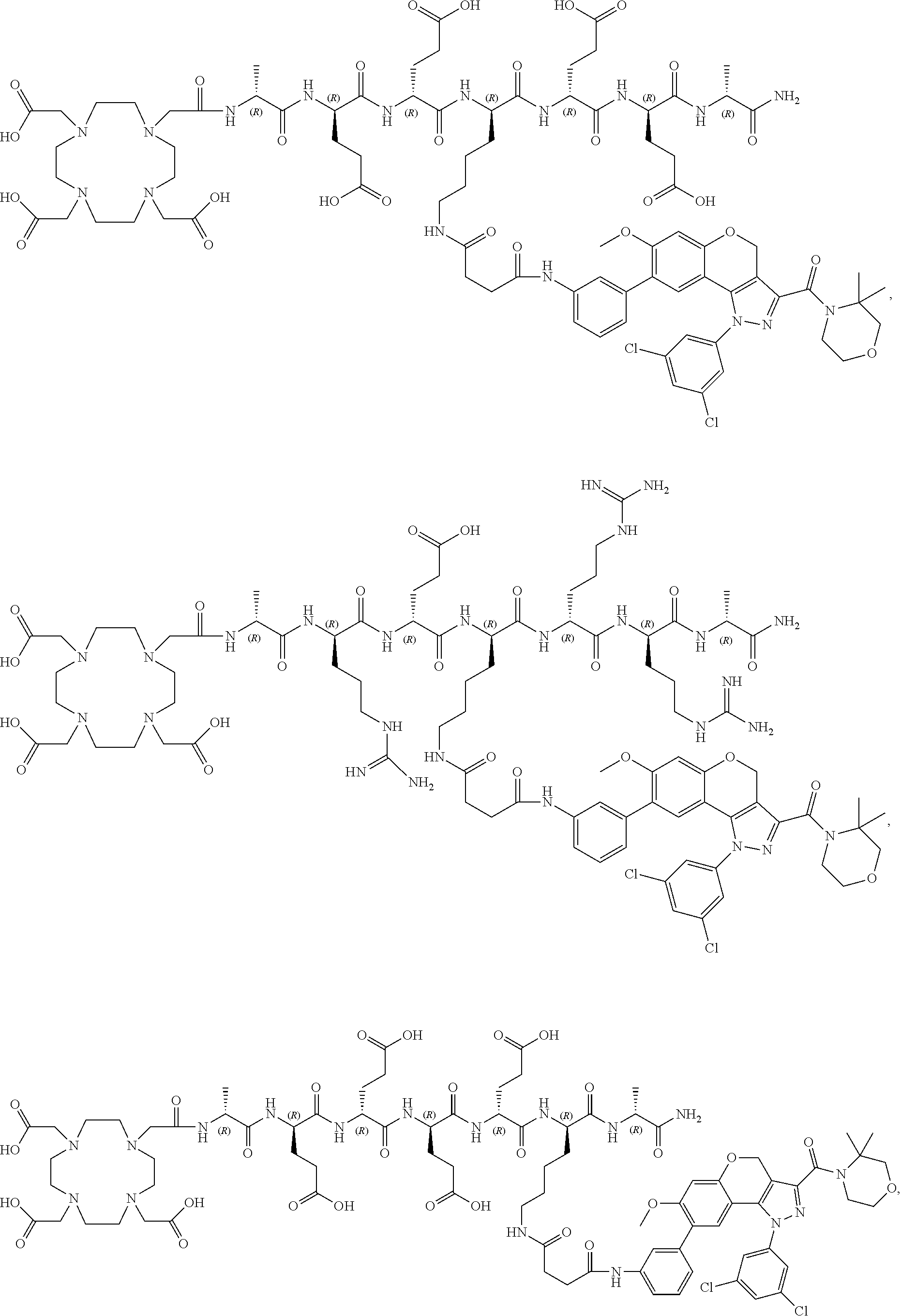

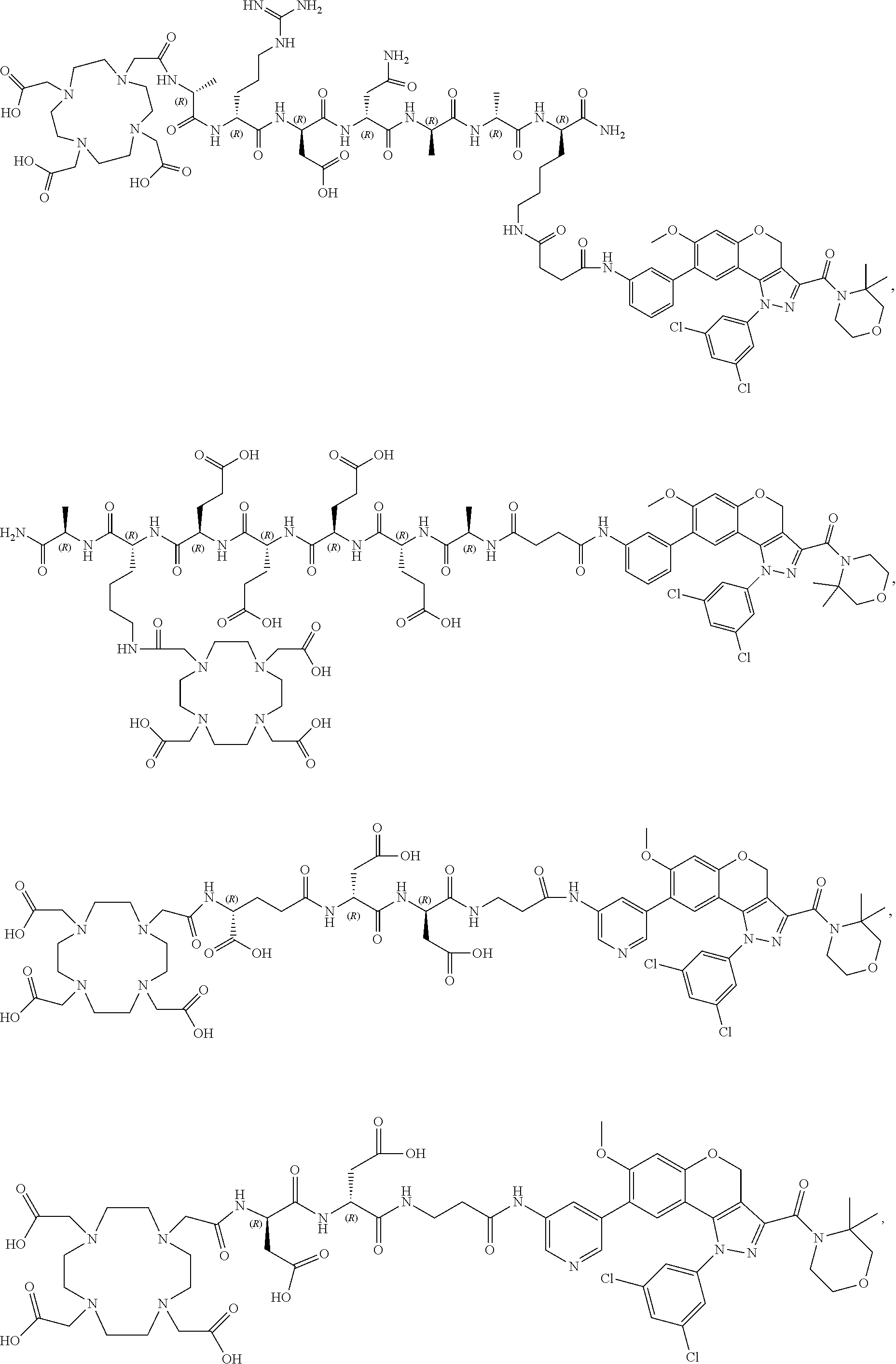

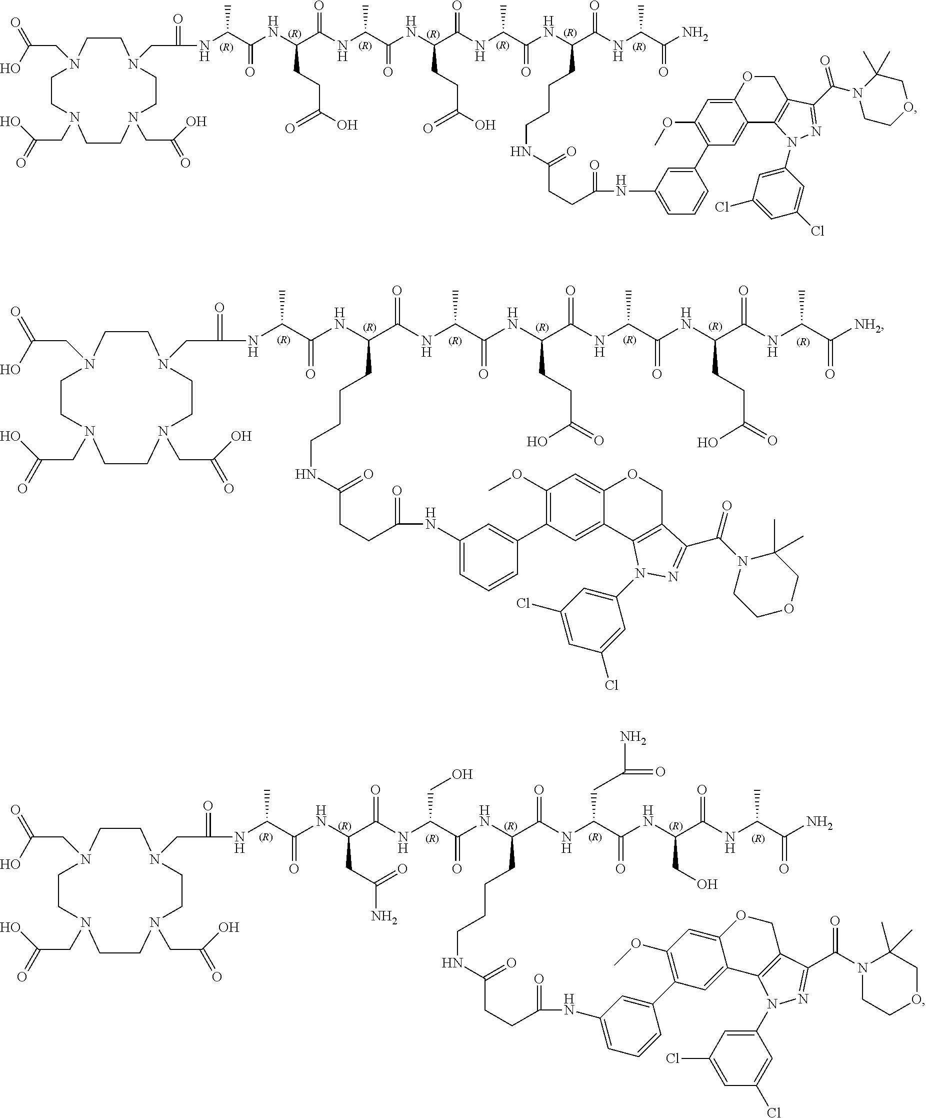

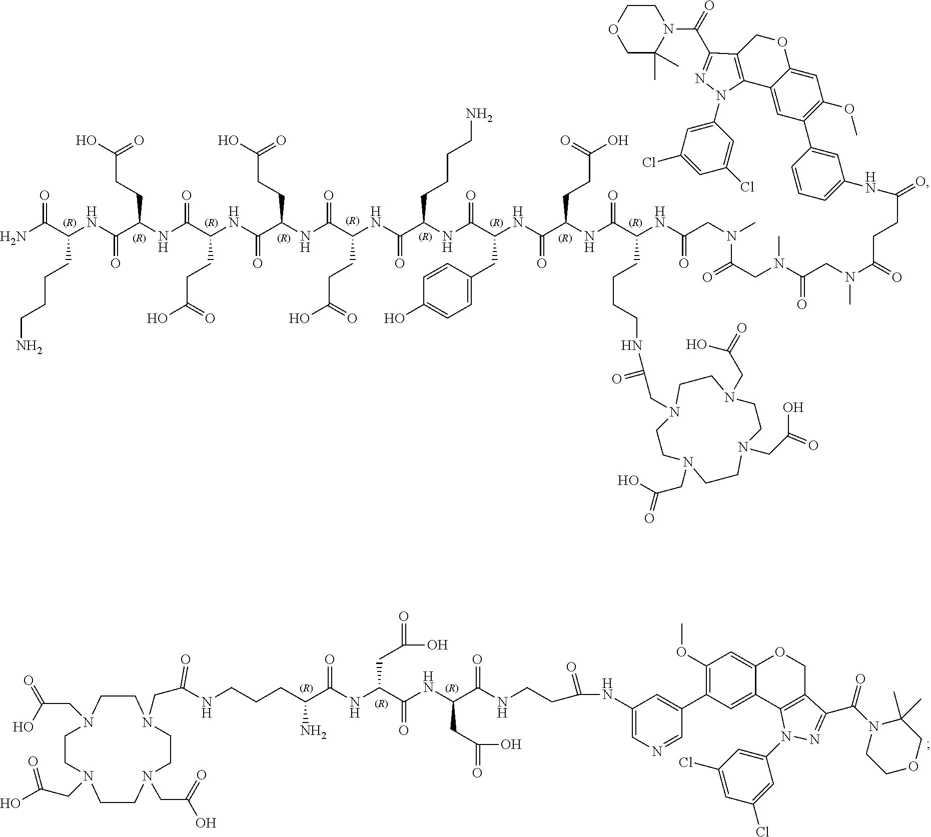

or radionuclide complex thereof.

In some embodiments, the compound of Formula (I) is compound 1, a pharmaceutically acceptable salt thereof, or radionuclide complex thereof. In some embodiments, the compound of Formula (I) is compound 2, a pharmaceutically acceptable salt thereof, or a radionuclide complex thereof. In some embodiments, the compound of Formula (I) is compound 3, a pharmaceutically acceptable salt thereof, or a radionuclide complex thereof. In some embodiments, the compound of Formula (I) is compound 4, a pharmaceutically acceptable salt thereof, or a radionuclide complex thereof. In some embodiments, the compound of Formula (I) is compound 5, a pharmaceutically acceptable salt thereof, or a radionuclide complex thereof. In some embodiments, the compound of Formula (I) is compound 6, a pharmaceutically acceptable salt thereof, or a radionuclide complex thereof. In some embodiments, the compound of Formula (I) is compound 7, a pharmaceutically acceptable salt thereof, or a radionuclide complex thereof.

In some embodiments, the compound of Formula (I) is compound 8, a pharmaceutically acceptable salt thereof, or a radionuclide complex thereof. In some embodiments, the compound of Formula (I) is compound 9, a pharmaceutically acceptable salt thereof, or a radionuclide complex thereof. In some embodiments, the compound of Formula (I) is compound 10, a pharmaceutically acceptable salt thereof, or a radionuclide complex thereof. In some embodiments, the compound of Formula (I) is compound 11, a pharmaceutically acceptable salt thereof, or a radionuclide complex thereof. In some embodiments, the compound of Formula (I) is compound 12, a pharmaceutically acceptable salt thereof, or a radionuclide complex thereof. In some embodiments, the compound of Formula (I) is compound 14, a pharmaceutically acceptable salt thereof, or a radionuclide complex thereof. In some embodiments, the compound of Formula (I) is compound 15, a pharmaceutically acceptable salt thereof, or a radionuclide complex thereof. In some embodiments, the compound of Formula (I) is compound 16, a pharmaceutically acceptable salt thereof, or a radionuclide complex thereof. In some embodiments, the compound of Formula (I) is compound 17, a pharmaceutically acceptable salt thereof, or a radionuclide complex thereof. In some embodiments, the compound of Formula (I) is compound 18, a pharmaceutically acceptable salt thereof, or a radionuclide complex thereof. In some embodiments, the compound of Formula (I) is compound 18, a pharmaceutically acceptable salt thereof, or a radionuclide complex thereof. In some embodiments, the compound of Formula (I) is compound 19, a pharmaceutically acceptable salt thereof, or a radionuclide complex thereof. In some embodiments, the compound of Formula (I) is compound 20, a pharmaceutically acceptable salt thereof, or a radionuclide complex thereof. In some embodiments, the compound of Formula (I) is compound 21, a pharmaceutically acceptable salt thereof, or a radionuclide complex thereof. In some embodiments, the compound of Formula (I) is compound 22, a pharmaceutically acceptable salt thereof, or a radionuclide complex thereof. In some embodiments, the compound of Formula (I) is compound 23, a pharmaceutically acceptable salt thereof, or a radionuclide complex thereof. In some embodiments, the compound of Formula (I) is compound 24, a pharmaceutically acceptable salt thereof, or a radionuclide complex thereof. In some embodiments, the compound of Formula (I) is compound 25, a pharmaceutically acceptable salt thereof, or a radionuclide complex thereof. In some embodiments, the compound of Formula (I) is compound 26, a pharmaceutically acceptable salt thereof, or a radionuclide complex thereof. In some embodiments, the compound of Formula (I) is compound 27, a pharmaceutically acceptable salt thereof, or a radionuclide complex thereof. In some embodiments, the compound of Formula (I) is compound 28, a pharmaceutically acceptable salt thereof, or a radionuclide complex thereof. In some embodiments, the compound of Formula (I) is compound 29, a pharmaceutically acceptable salt thereof, or a radionuclide complex thereof. In some embodiments, the compound of Formula (I) is compound 30, a pharmaceutically acceptable salt thereof, or a radionuclide complex thereof. In some embodiments, the compound of Formula (I) is compound 31, a pharmaceutically acceptable salt thereof, or a radionuclide complex thereof. In some embodiments, the compound of Formula (I) is compound 32, a pharmaceutically acceptable salt thereof, or a radionuclide complex thereof. In some embodiments, the compound of Formula (I) is compound 33, a pharmaceutically acceptable salt thereof, or a radionuclide complex thereof. In some embodiments, the compound of Formula (I) is compound 34, a pharmaceutically acceptable salt thereof, or a radionuclide complex thereof. In some embodiments, the compound of Formula (I) is compound 36, a pharmaceutically acceptable salt thereof, or a radionuclide complex thereof. In some embodiments, the compound of Formula (I) is compound 37, a pharmaceutically acceptable salt thereof, or a radionuclide complex thereof. In some embodiments, the compound of Formula (I) is compound 38, a pharmaceutically acceptable salt thereof, or a radionuclide complex thereof. In some embodiments, the compound of Formula (I) is compound 39, a pharmaceutically acceptable salt thereof, or a radionuclide complex thereof. In some embodiments, the compound of Formula (I) is compound 40, a pharmaceutically acceptable salt thereof, or a radionuclide complex thereof. In some embodiments, the compound of Formula (I) is compound 41, a pharmaceutically acceptable salt thereof, or a radionuclide complex thereof. In some embodiments, the compound of Formula (I) is compound 42, a pharmaceutically acceptable salt thereof, or a radionuclide complex thereof. In some embodiments, the compound of Formula (I) is compound 43, a pharmaceutically acceptable salt thereof, or a radionuclide complex thereof. In some embodiments, the compound of Formula (I) is compound 44, a pharmaceutically acceptable salt thereof, or a radionuclide complex thereof. In some embodiments, the compound of Formula (I) is compound 45, a pharmaceutically acceptable salt thereof, or a radionuclide complex thereof. In some embodiments, the compound of Formula (I) is compound 46, a pharmaceutically acceptable salt thereof, or a radionuclide complex thereof. In some embodiments, the compound of Formula (I) is compound 47, a pharmaceutically acceptable salt thereof, or a radionuclide complex thereof. In some embodiments, the compound of Formula (I) is compound 48, a pharmaceutically acceptable salt thereof, or a radionuclide complex thereof. In some embodiments, the compound of Formula (I) is compound 49, a pharmaceutically acceptable salt thereof, or a radionuclide complex thereof. In some embodiments, the compound of Formula (I) is compound 50, a pharmaceutically acceptable salt thereof, or a radionuclide complex thereof. In some embodiments, the compound of Formula (I) is compound 51, a pharmaceutically acceptable salt thereof, or a radionuclide complex thereof. In some embodiments, the compound of Formula (I) is compound 52, a pharmaceutically acceptable salt thereof, or a radionuclide complex thereof. In some embodiments, the compound of Formula (I) is compound 53, a pharmaceutically acceptable salt thereof, or a radionuclide complex thereof. In some embodiments, the compound of Formula (I) is compound 54, a pharmaceutically acceptable salt thereof, or a radionuclide complex thereof. In some embodiments, the compound of Formula (I) is compound 55, a pharmaceutically acceptable salt thereof, or a radionuclide complex thereof. In some embodiments, the compound of Formula (I) is compound 56, a pharmaceutically acceptable salt thereof, or a radionuclide complex thereof. In some embodiments, the compound of Formula (I) is compound 57, a pharmaceutically acceptable salt thereof, or a radionuclide complex thereof. In some embodiments, the compound of Formula (I) is compound 58, a pharmaceutically acceptable salt thereof, or a radionuclide complex thereof. In some embodiments, the compound of Formula (I) is compound 59, a pharmaceutically acceptable salt thereof, or a radionuclide complex thereof. In some embodiments, the compound of Formula (I) is compound 60, a pharmaceutically acceptable salt thereof, or a radionuclide complex thereof. In some embodiments, the compound of Formula (I) is compound 61, a pharmaceutically acceptable salt thereof, or a radionuclide complex thereof. In some embodiments, the compound of Formula (I) is compound 62, a pharmaceutically acceptable salt thereof, or a radionuclide complex thereof. In some embodiments, the compound of Formula (I) is compound 63, a pharmaceutically acceptable salt thereof, or a radionuclide complex thereof. In some embodiments, the compound of Formula (I) is compound 64, a pharmaceutically acceptable salt thereof, or a radionuclide complex thereof. In some embodiments, the compound of Formula (I) is compound 65, a pharmaceutically acceptable salt thereof, or a radionuclide complex thereof. In some embodiments, the compound of Formula (I) is compound 66, a pharmaceutically acceptable salt thereof, or a radionuclide complex thereof. In some embodiments, the compound of Formula (I) is compound 67, a pharmaceutically acceptable salt thereof, or a radionuclide complex thereof. In some embodiments, the compound of Formula (I) is compound 68, a pharmaceutically acceptable salt thereof, or a radionuclide complex thereof. In some embodiments, the compound of Formula (I) is compound 69, a pharmaceutically acceptable salt thereof, or a radionuclide complex thereof. In some embodiments, the compound of Formula (I) is compound 70, a pharmaceutically acceptable salt thereof, or a radionuclide complex thereof. In some embodiments, the compound of Formula (I) is compound 71, a pharmaceutically acceptable salt thereof, or a radionuclide complex thereof. In some embodiments, the compound of Formula (I) is compound 72, a pharmaceutically acceptable salt thereof, or a radionuclide complex thereof. In some embodiments, the compound of Formula (I) is compound 73, a pharmaceutically acceptable salt thereof, or a radionuclide complex thereof. In some embodiments, the compound of Formula (I) is compound 74, a pharmaceutically acceptable salt thereof, or a radionuclide complex thereof. In some embodiments, the compound of Formula (I) is compound 75, a pharmaceutically acceptable salt thereof, or a radionuclide complex thereof. In some embodiments, the compound of Formula (I) is compound 76, a pharmaceutically acceptable salt thereof, or a radionuclide complex thereof. In some embodiments, the compound of Formula (I) is compound 77, a pharmaceutically acceptable salt thereof, or a radionuclide complex thereof. In some embodiments, the compound of Formula (I) is compound 78, a pharmaceutically acceptable salt thereof, or a radionuclide complex thereof. In some embodiments, the compound of Formula (I) is compound 79, a pharmaceutically acceptable salt thereof, or a radionuclide complex thereof. In some embodiments, the compound of Formula (I) is compound 80, a pharmaceutically acceptable salt thereof, or a radionuclide complex thereof. In some embodiments, the compound of Formula (I) is compound 81, a pharmaceutically acceptable salt thereof, or a radionuclide complex thereof. In some embodiments, the compound of Formula (I) is compound 82, a pharmaceutically acceptable salt thereof, or a radionuclide complex thereof. In some embodiments, the compound of Formula (I) is compound 83, a pharmaceutically acceptable salt thereof, or a radionuclide complex thereof. In some embodiments, the compound of Formula (I) is compound 84, a pharmaceutically acceptable salt thereof, or a radionuclide complex thereof. In some embodiments, the compound of Formula (I) is compound 85, a pharmaceutically acceptable salt thereof, or a radionuclide complex thereof. In some embodiments, the compound of Formula (I) is compound 86, a pharmaceutically acceptable salt thereof, or a radionuclide complex thereof. In some embodiments, the compound of Formula (I) is compound 87, a pharmaceutically acceptable salt thereof, or a radionuclide complex thereof. In some embodiments, the compound of Formula (I) is compound 88, a pharmaceutically acceptable salt thereof, or a radionuclide complex thereof. In some embodiments, the compound of Formula (I) is compound 89, a pharmaceutically acceptable salt thereof, or a radionuclide complex thereof. In some embodiments, the compound of Formula (I) is compound 90, a pharmaceutically acceptable salt thereof, or a radionuclide complex thereof. In some embodiments, the compound of Formula (I) is compound 91, a pharmaceutically acceptable salt thereof, or a radionuclide complex thereof. In some embodiments, the compound of Formula (I) is compound 92, a pharmaceutically acceptable salt thereof, or a radionuclide complex thereof. In some embodiments, the compound of Formula (I) is compound 93, a pharmaceutically acceptable salt thereof, or a radionuclide complex thereof. In some embodiments, the compound of Formula (I) is compound 94, a pharmaceutically acceptable salt thereof, or a radionuclide complex thereof. In some embodiments, the compound of Formula (I) is compound 95, a pharmaceutically acceptable salt thereof, or a radionuclide complex thereof. In some embodiments, the compound of Formula (I) is compound 96, a pharmaceutically acceptable salt thereof, or a radionuclide complex thereof. In some embodiments, the compound of Formula (I) is compound 97, a pharmaceutically acceptable salt thereof, or a radionuclide complex thereof. In some embodiments, the compound of Formula (I) is compound 98, a pharmaceutically acceptable salt thereof, or a radionuclide complex thereof. In some embodiments, the compound of Formula (I) is compound 99, a pharmaceutically acceptable salt thereof, or a radionuclide complex thereof. In some embodiments, the compound of Formula (I) is compound 100, a pharmaceutically acceptable salt thereof, or a radionuclide complex thereof. In some embodiments, the compound of Formula (I) is compound 101, a pharmaceutically acceptable salt thereof, or a radionuclide complex thereof. In some embodiments, the compound of Formula (I) is compound 102, a pharmaceutically acceptable salt thereof, or a radionuclide complex thereof. In some embodiments, the compound of Formula (I) is compound 103, a pharmaceutically acceptable salt thereof, or a radionuclide complex thereof. In some embodiments, the compound of Formula (I) is compound 104, a pharmaceutically acceptable salt thereof, or a radionuclide complex thereof.

In some embodiments, the compound of Formula (B) has one of the following structures, or a pharmaceutically acceptable salt thereof:

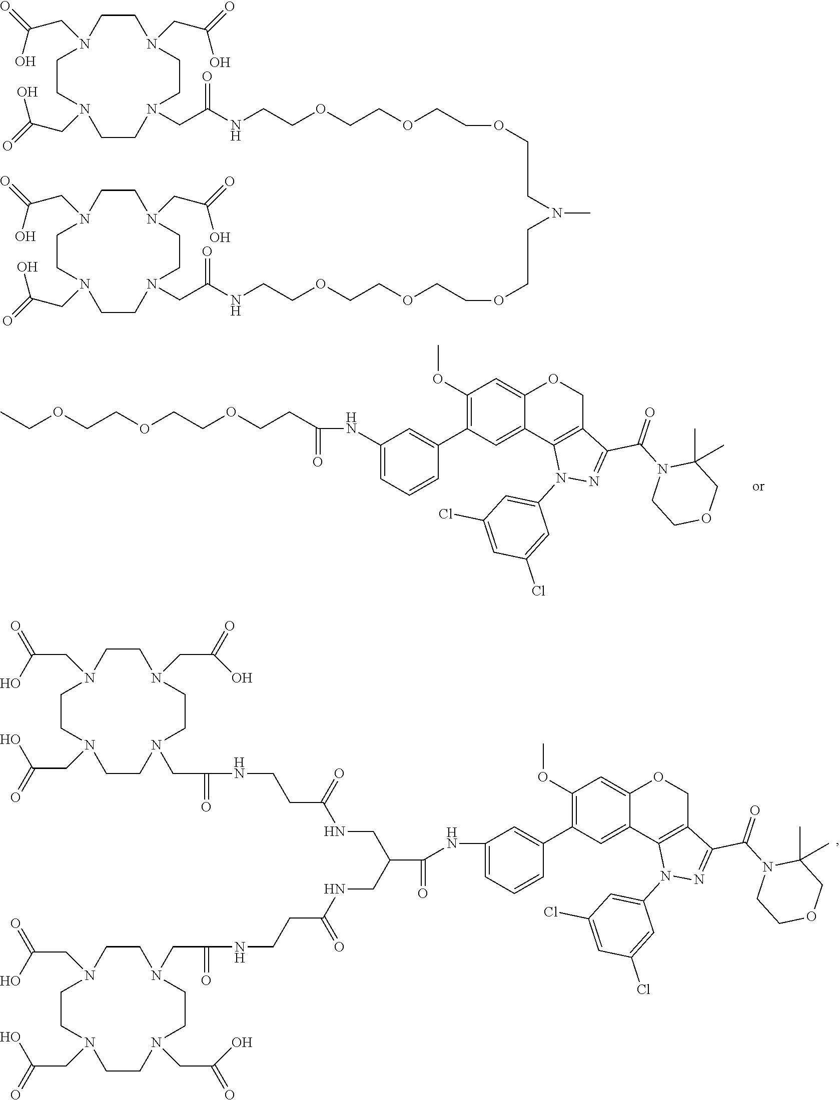

or radionuclide complex thereof.

In some embodiments, the compound of Formula (B) is compound 13, a pharmaceutically acceptable salt thereof, or radionuclide complex thereof. In some embodiments, the compound of Formula (B) is compound 35, a pharmaceutically acceptable salt thereof, or radionuclide complex thereof.

Any combination of the groups described above for the various variables is contemplated herein. Throughout the specification, groups and substituents thereof are chosen by one skilled in the field to provide stable moieties and compounds.

### Synthesis of Compounds

Compounds described herein are synthesized using standard synthetic techniques or using methods known in the art in combination with methods described herein.

Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I), Formula (B), or Formula (A), with an acid. In some embodiments, the compound of Formula (I), Formula (B), or Formula (A), (i.e., free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (-L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (-L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I), Formula (B), or Formula (A), is prepared as a chloride salt, sulfate salt, bromide salt, mesylate salt, maleate salt, citrate salt or phosphate salt.

In some embodiments, pharmaceutically acceptable salts are obtained by reacting a compound of Formula (I), Formula (B), or Formula (A), with a base. In some embodiments, the compound of Formula (I), Formula (B), or Formula (A), is acidic and is reacted with a base. In such situations, an acidic proton of the compound of Formula (I), Formula (B), or Formula (A), is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, sites on the organic radicals (e.g., alkyl groups, aromatic rings) of compounds of Formula (I), Formula (B), or Formula (A), are deuterated.

In some embodiments, the compounds of Formula (I), Formula (B), or Formula (A), possess one or more stereocenters and each stereocenter exists independently in either the R or S configuration. In some embodiments, the compound of Formula (I), Formula (B), or Formula (A), exists in the R configuration. In some embodiments, the compound of Formula (I), Formula (B), or Formula (A), exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I), Formula (B), or Formula (A), are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. See for example Design of Prodrugs, Bundgaard, A. Ed., Elsevier, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

A “metabolite” of a compound disclosed herein is a derivative of that compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to, delivery via parenteral routes (including injection or infusion, and subcutaneous).

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use.

### Methods of Treatment

In some embodiments, the methods comprise administering to a subject a therapeutically effective amount of a compound of Formula (I), Formula (B), or Formula (A), or a pharmaceutically acceptable salt or solvate thereof. In some embodiments, the compound of Formula (I), Formula (B), or Formula (A), or pharmaceutically acceptable salt or solvate thereof is administered in a pharmaceutical composition. In some embodiments, the subject has cancer. In some embodiments, the cancer is a solid tumor. In some embodiments, the subject has a noncancerous tumor. In some embodiments, the subject has an adenoma.

In some embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (I), Formula (B), or Formula (A), or a pharmaceutically acceptable salt or solvate thereof. In some embodiments, the compound of Formula (I) or pharmaceutically acceptable salt or solvate thereof releases a number of alpha particles by natural radioactive decay. In some embodiments, the released alpha particles are sufficient to kill the tumor cell. In some embodiments, the released alpha particles are sufficient to stop cell growth. In some embodiments, the tumor cell is a malignant tumor cell. In some embodiments, the tumor cell is a benign tumor cell. In some embodiments, the method comprises killing a tumor cell with a beta-particle emitting radionuclide. In some embodiments, the method comprises killing a tumor cell with an alpha-particle emitting radionuclide. In some embodiments, the method comprises killing a tumor cell with a gamma-particle emitting radionuclide.

In one aspect, provided herein are methods and compositions for treating cancers.

In one aspect, provided herein are methods and compositions for treating an adenoma.

In one aspect, provided herein are methods and compositions for treating a carcinoma.

In one aspect, provided herein is a method for identifying tissues or organs in a mammal that overexpress FSHR comprising: (i) administering to the mammal a compound of Formula (I), Formula (B), or Formula (A; and (ii) performing single-photon emission computerized tomography (SPECT) or positron emission tomography (PET) analysis on the mammal. In some embodiments, the method comprises: (i) administering to the mammal a compound of Formula (I), Formula (B), or Formula (A; and (ii) performing positron emission tomography (PET) analysis on the mammal.

In some embodiments, the mammal was diagnosed with cancer. In some embodiments, the tissues in the mammal that overexpress FSHR are tumors.

In some embodiments, compounds of Formula (I), Formula (B), or Formula (A), disclosed herein are used in a method for in vivo imaging of a subject. In some embodiments, the method includes the steps of:
(i) administering to the mammal a compound of Formula (I), Formula (B), or Formula (A);
(ii)_{w}aiting a sufficient amount of time to allow the compound of Formula (I), Formula (B), or Formula (A), to accumulate at a tissue or cell site to be imaged; and
(iii) imaging the cells or tissues with a non-invasive imaging technique.

In some embodiments, the non-invasive imaging technique is single-photon emission computerized tomography (SPECT) or positron emission tomography (PET) analysis. In some embodiments, the non-invasive imaging technique is single-photon emission computerized tomography (SPECT). In some embodiments, the non-invasive imaging technique is selected from positron emission tomography imaging, or positron emission tomography with computed tomography imaging, and positron emission tomography with magnetic resonance imaging.

### Methods of Dosing and Treatment Regimens

In one embodiment, compounds of Formula (I), Formula (B), or Formula (A), or a pharmaceutically acceptable salt thereof, are used in the preparation of medicaments for the treatment of tumors in a mammal. Methods for treating any of the diseases or conditions described herein in a mammal in need of such treatment, involves administration of pharmaceutical compositions that include at least one compound of Formula (I) or a pharmaceutically acceptable salt thereof, in therapeutically effective amounts to said mammal.

In certain embodiments, the compositions containing the compound(s) described herein are administered for diagnostic and/or therapeutic treatments.

The amount of a given agent that corresponds to such an amount varies depending upon factors such as the particular conjugate, specific cancer or tumor to be treated (and its severity), the identity (e.g., weight, sex) of the subject or host in need of treatment, but nevertheless is determined according to the particular circumstances surrounding the case, including, e.g., the specific conjugate being administered, the route of administration, the condition being treated, and the subject or host being treated. Optimal doses are generally determined using experimental models and/or clinical trials. The optimal dose depends upon the body mass, weight, or blood volume of the subject.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans.

The amount of a compound of Formula (I), Formula (B), or Formula (A), or pharmaceutically acceptable salts thereof and/or pharmaceutical compositions that are administered are sufficient to deliver a therapeutically effective dose to the particular subject. In some embodiments, dosages of a compound of Formula (I), Formula (B), or Formula (A), are between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of a compound of Formula (I), Formula (B), or Formula (A), or a pharmaceutically acceptable salt thereof described herein for methods of treating a disease as described herein is about 0.001 mg/kg to about 1 mg/kg body weight of the subject per dose. In some embodiments, the dose of a compound of Formula (I), Formula (B), or Formula (A), or a pharmaceutically acceptable salt thereof described herein for the described methods is about 0.001 mg to about 1000 mg per dose for the subject being treated. In some embodiments, a compound of Formula (I), Formula (B), or Formula (A), or a pharmaceutically acceptable salt thereof described herein is administered to a subject at a dosage of from about 0.01 mg to about 500 mg, from about 0.01 mg to about 100 mg, or from about 0.01 mg to about 50 mg.

In some embodiments, a compound of Formula (I), Formula (B), or Formula (A), or a pharmaceutically acceptable salt thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole.

In some embodiments, a compound of Formula (I), Formula (B), or Formula (A), or a pharmaceutically acceptable salt thereof described herein is administered to a subject at a dosage of about 0.01 Gbq to about 1000 Gbq, about 0.5 Gbq to about 100 Gbq, or about 1 Gbq to about 50 Gbq.

In some embodiments, the dose is administered once a day, 1 to 3 times a week, 1 to 4 times a month, or 1 to 12 times a year.

In any of the aforementioned aspects are further embodiments in which the effective amount of the compound of Formula (I), Formula (B), or Formula (A), or a pharmaceutically acceptable salt thereof, is: (a) systemically administered to the mammal; and/or (b) intravenously administered to the mammal; and/or (c) administered by injection to the mammal.

In certain instances, it is appropriate to administer at least one compound of Formula (I), Formula (B), or Formula (A), or a pharmaceutically acceptable salt thereof, in combination with one or more other therapeutic agents.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As discussed herein, “small molecule” is a low molecular weight organic compound, that has a molecular weight less than 1000 daltons and does not contain >3 consecutive amino acid moieties. In some embodiments, the molecular weight of the small molecule is ≤900 daltons. In some embodiments, the molecular weight of the small molecule is ≤800 daltons. In some embodiments, the molecular weight of the small molecule is ≤700 daltons. In some embodiments, the molecular weight of the small molecule is ≤600 daltons.

As used herein, C₁-Cₓ includes C₁-C₂, C₁-C₃ . . . . C₁-Cₓ. By way of example only, a group designated as “C₁-C₆” indicates that there are one to six carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e., a C₁-C₁₀ alkyl. Whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, the alkyl group is an “alkenyl” or “alkynyl” group.

An “alkylene” group refers to a divalent alkyl radical. Any of the above-mentioned monovalent alkyl groups may be an alkylene by abstraction of a second hydrogen atom from the alkyl. In some embodiments, an alkylene is a C₁-C₆ alkylene. In other embodiments, an alkylene is a C₁-C₄ alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—. In some embodiments, an alkylene is —CH₂CH₂—.

An “alkoxy” group refers to an (alkyl)O—group, where alkyl is as defined herein.

The term “alkenyl” refers to a type of alkyl group in which at least one carbon-carbon double bond is present. In one embodiment, an alkenyl group has the formula —C(R)—CR₂, wherein R refers to the remaining portions of the alkenyl group, which may be the same or different. In some embodiments, each R is independently H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include-CH—CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula-C═C—R, wherein R refers to the remaining portion of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include-C═CH, —C═CCH₃—C═CCH₂CH₃, —CH₂C═CH.

The term “heteroalkyl” refers to an alkyl group in which one or more skeletal atoms of the alkyl are selected from an atom other than carbon, e.g., oxygen, nitrogen (e.g., —NH—, —N (alkyl)-), sulfur, or combinations thereof. In some embodiments, the “heteroalkyl” group has 2 to 10 atoms in the backbone, which include a combination of carbon atoms and heteroatoms (e.g. N, O, S), i.e., a 2 to 10 membered heteroalkyl. In some embodiments, the heteroalkyl is attached to the rest of the molecule at a carbon atom of the heteroalkyl. In one embodiment, a heteroalkyl is a 2 to 8 membered heteroalkyl.

A “heteroalkylene” group refers to a divalent alkyl radical derived from heteroalkyl, as exemplified, but not limited by, —CH₂—CH₂—O—CH₂—CH₂—and —CH₂—O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula-C(═O)O—represents both-C(═O)O—and —OC(═O)—. Additionally, the formula-C(═O)NH-represents both-C(═O)NH—and —NHC(═O)—.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀ aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e., skeletal atoms) is a carbon atom. In some embodiments, cycloalkyls are spirocyclic or bridged cycloalkyls. In some embodiments, cycloalkyls are optionally fused with an aromatic ring, and the point of attachment is at a carbon that is not an aromatic ring carbon atom. Cycloalkyl groups include groups having from 3 to 12 ring atoms. In some embodiments, cycloalkyl groups are selected from cyclopropyl, cyclobutyl, cyclopentyl, cyclopentenyl, cyclohexyl, cyclohexenyl, cycloheptyl, cyclooctyl, spiro[2.2]pentyl, norbornyl and bicycle [1.1.1]pentyl. In some embodiments, a cycloalkyl is a C₃-C₆ cycloalkyl. In some embodiments, a cycloalkyl is a C₃-C₄ cycloalkyl. In some embodiments, a cycloalkyl is a C₅-C₆ cycloalkyl.

The term “halo” or, alternatively, “halogen” or “halide” means fluoro, chloro, bromo, or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆ fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 12 atoms in its ring system, and with the proviso that any ring does not contain two adjacent O or S atoms. Non-aromatic heterocyclic groups (also known as heterocycloalkyls) include rings having 3 to 12 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1 (2H)-onyl, 3,4-dihydroquinolin-2 (1H)-onyl, isoindoline-1,3-dithionyl, benzo[d]oxazol-2 (3H)-onyl, 1H-benzo[d]imidazol-2 (3H)-onyl, benzo[d]thiazol-2 (3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen, and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1˜4 N atoms in the ring. In some embodiments, a heteroaryl contains 0-4 N atoms, 0-1 O atoms, and 0-1 S atoms in the ring. In some embodiments, a heteroaryl contains 1˜4 N atoms, 0-1 O atoms, and 0-1 S atoms in the ring. In some embodiments, a heteroaryl contains 1 O atom. In some embodiments, a heteroaryl contains 1 S atom in the ring. In some embodiments, heteroaryl is a 5 to 10 membered heteroaryl. In some embodiments, a monocyclic heteroaryl is a 5 to 6 membered heteroaryl. In some embodiments, a monocyclic heteroaryl is a 5-membered heteroaryl. In some embodiments, a monocyclic heteroaryl is a 6-membered heteroaryl. In some embodiments, bicyclic heteroaryl is a 10 membered heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a 3 to 12 membered heterocycloalkyl. In another aspect, a heterocycloalkyl is a 5 to 10 membered heterocycloalkyl. In some embodiments, a heterocycloalkyl is a 5 membered heterocycloalkyl. In some embodiments, a heterocycloalkyl is a 6 membered heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1˜4 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of a larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH (alkyl), —N(alkyl)₂, —OH, —CO₂H, —CO₂alkyl, —C(═O)NH₂, —C(—O)NH (alkyl), —C(—O)N (alkyl)₂, —S(═O)₂NH₂, —S(—O)₂NH (alkyl), —S(═O)₂N (alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, -SC₁-C₄ alkyl, —S(═O) C₁-C₄ alkyl, and —S(═O)₂C₁-C₄ alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically. In some embodiments, “treating” as used herein, includes alleviating, abating or ameliorating at least one symptom of a disease or condition, preventing additional symptoms, arresting the development or progression of the disease or condition, causing regression of the disease or condition, or stopping at least one symptom of the disease or condition. For example, in some embodiments, treating comprises stopping the growth of, reducing the size of, reducing the abundance of, reducing the distribution within the mammal of, tumor cells expressing FSHR.

### Examples

The following examples are provided for illustrative purposes only and not to limit the scope of the claims provided herein.

### Abbreviations

AcOH: acetic acid; FA: formic acid; PivOH: pivalic acid; TFA: trifluoroacetic acid;
ACN or MeCN or CH₃CN: acetonitrile;
Boc₂O: di-tert-butyl carbonate;
H₂O: water; brine: saturated NaCl solution;
MeOH: methanol; EtOH: ethanol; 1-BuOH: 1-butanol;
DBU: 1,8-diazabicyclo[5.4.0]undec-7-ene;
DCC: N,N′-dicyclohexylcarbodiimide; EDCI: 1-Ethyl-3-(3-dimethylaminopropyl) carbodiimide;
DCM: dichloromethane; DCE: dichloroethane;
DIEA or DIPEA: N,N-diisopropylethylamine; TEA: triethylamine;
DMAP: 4-dimethylaminopyridine;
DMF: dimethylformamide; DMSO: dimethyl sulfoxide;
DOTA-tris(t-Bu) ester NHS ester: 2,2′,2″-(10-(2-((2,5-dioxopyrrolidin-1-yl)oxy)-2-oxoethyl)-1,4,7,10-tetraazacyclododecane-1,4,7-triyl)triacetic acid;
ELSD: evaporative light scattering detector;
Et₂O: diethyl ether; PE: petroleum ether; THF: tetrahydrofuran;
EtOAc or EA: ethyl acetate;
FDPP: Pentafluorophenyl diphenylphosphinate; HATU: 1-[Bis(dimethylamino)methylene]-1H-1,2,3-triazolo[4,5-b]pyridinium 3-oxide hexafluorophosphate; HBTU: N,N,N′,N′-Tetramethyl-O-(1H-benzotriazol-1-yl)uronium hexafluorophosphate; HOBt: hydroxybenzotriazole;
HCl: hydrochloric acid or hydrochloride;
InCl₃: indium trichloride; LuCl₃: lutetium (III) chloride;
K₂CO₃: potassium carbonate; Na₂CO₃: sodium carbonate;
LCMS: Liquid chromatography-mass spectrometry; MS: mass spectrometry;
LDA: lithium diisopropylamide;
MPLC: Medium pressure liquid chromatography;
NaOH: sodium hydroxide; Na₂SO₄: sodium sulfate; NaHSO₄: sodium hydrogen sulfate;
NBS: N-bromosuccinimide;
NH₄Cl: ammonium chloride;
NMI: N-methylimidazole;
NMM: N-methyl morpholine; NMP: N-methyl-2-pyrrolidone;
Pd(dppf)Cl₂:1,1′-Bis(di-t-butylphosphino) ferrocene palladium dichloride;
Pd(PPh₃)₄: Palladium-tetrakis(triphenylphosphine);
Prep-HPLC: preparative high-performance liquid chromatography;
PyBop: (Benzotriazol-1-yloxy)tripyrrolidinophosphonium hexafluorophosphate;
TCFH: chloro-N,N,N′,N′-tetramethylformamidinium hexafluorophosphate;
TIPS: triisopropylsilane; TMSI: trimethylsilyl iodide; PhSiH₃: phenylsilane;
rt: room temperature;
h or hr: hour; hrs: hours; min: minute;
mg: milligrams; kg: kilograms; mL or ml: milliliter;
Eq: equivalents; mmol: millimole; mol: moles;
UV: ultraviolet
General Analytical Methods:
Prep-HPLC with DAC: The crude product was purified by DAC-HPLC: Column, YMC-C18, 150-250 nm, 10 μm; Mobile phase, Water (0.05% TFA) and ACN (25% ACN up to 65% in 8 min); Total flow rate, 120 mL/min; Detector, UV 220 nm.

LC-MS analyses were carried out on a Shimadzu LCMS-2020 series equipped with a binary pump LC-20ADXR, micro vacuum degasser, standard auto sampler SIL-20AC XR, thermostatted column compartment CTO—20AC, variable wavelength detector SPD-M20A, and data were analyzed by Shimadzu LabSolutions standalone workstation software. HPLC solvents consisted of H₂O containing 0.05% ammonia (mobile phase A) and acetonitrile (mobile phase B). Conditions: An Ascentis Express C18 (2.6 μm, 3.0×50 mm) column was used with a flow rate of 1.2 mL/min. ¹H NMR spectra were recorded using a AVANCE III HD 300 MHz. Chemical shifts are reported in 8 (ppm) relative to TMS+Si (in DMSO—d6) as internal standard using Instrument model (Bruker TopSpin) unless otherwise noted.

### Synthesis of Compounds

### Example 1:2,2′,2″-(10-(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8,11,14,17,20-hexaoxa-2,23-diazapentacosan-25-yl)-1,4,7,10-tetraazacyclododecane-1,4,7-triyl)triacetic acid (Compound 1)

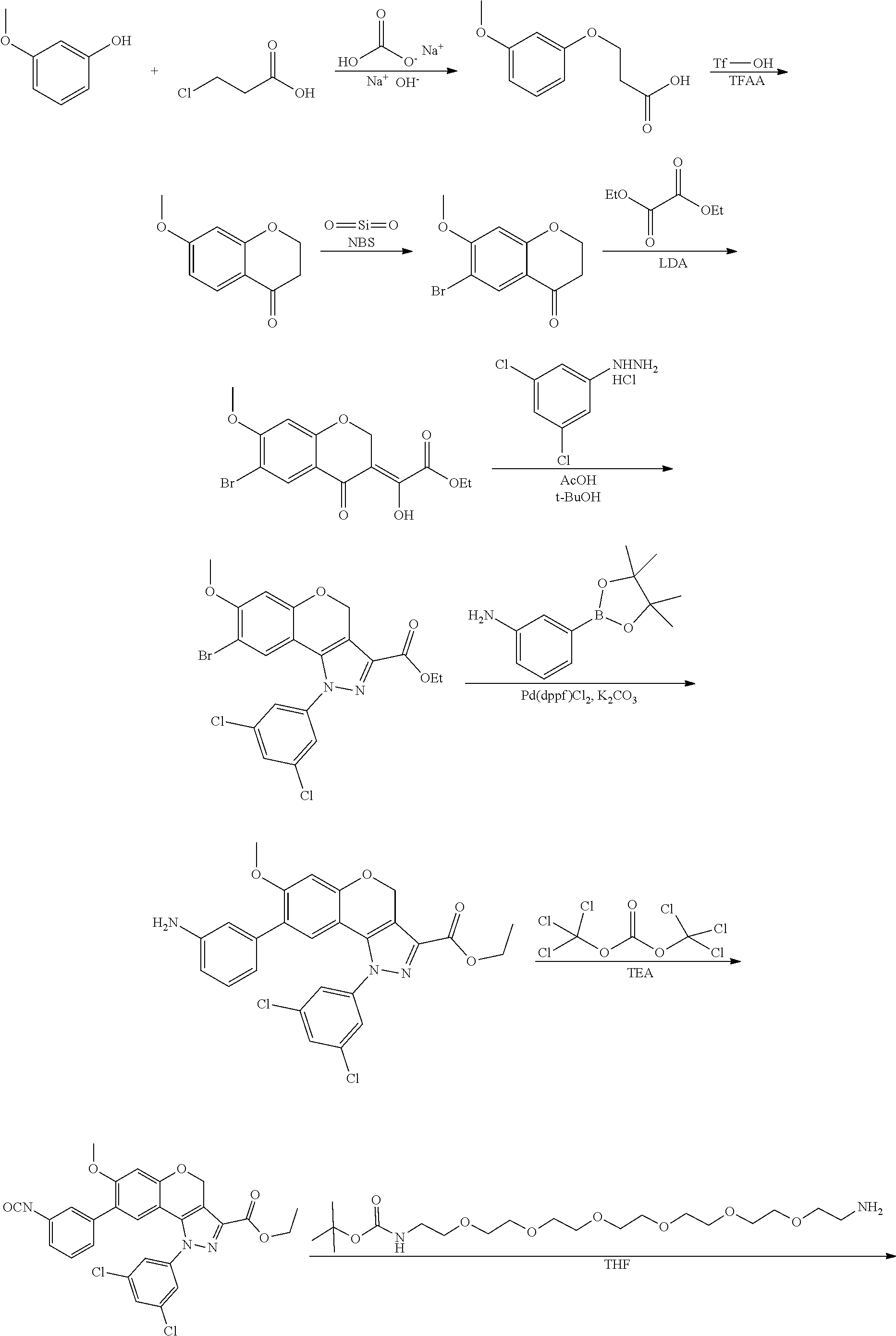

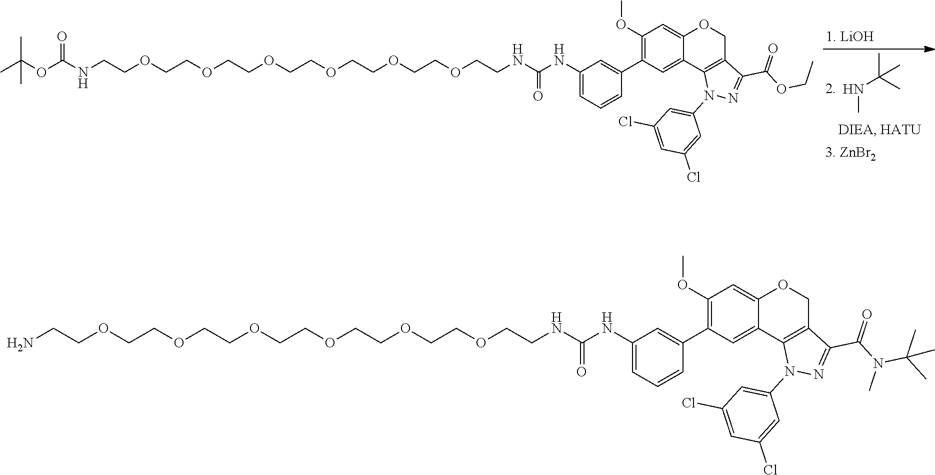

Step 1: Into a 500-mL round-bottom flask, was placed a mixture of 3-chloropropanoic acid (52 g, 1.2 Eq, 0.48 mol) in 100 ml water then a saturated solution of sodium hydrogen carbonate (81 g, 2.4 Eq, 0.96 mol)_{w} as added. The mixture was stirred at room temperature for 5 mins. 3-Methoxyphenol (50 g, 1 Eq, 0.40 mol)_{w} as added to a saturated solution of sodium hydroxide (39 g, 2.4 Eq, 0.98 mol), and the resulting mixture was added to the initial mixture. The reaction mixture was stirred at 100° C. for 3 hours. The mixture was adjusted to pH=5 using 2N HCl, extracted with EtOAc (200 mL×3), and the combined organic layers were washed with brine (150 mL), dried over anhydrous Na₂SO₄ and concentrated under reduced pressure. The crude product was purified by MPLC with the following conditions: Silica gel column 330 g, PE/EtOAc system, the ratio of EtOAc from 0% to 85% in 15 min, Flow rate: 40 mL/min; Wave Length: 254 nm. The collected fractions were concentrated under reduced pressure to give 3-(3-methoxyphenoxy) propanoic acid (12.5 g, 57 mmol, 14%, 90% Purity) as a yellow oil. MS: Calc'd for C₁₀H₁₂O₄:196.07, found [M+H]⁺: 197.2, 199.2.

Step 2: Into a 500-mL round bottom flask, was placed a mixture of 3-(3-methoxyphenoxy) propanoic acid (12.5 g, 1 Eq, 63.7 mmol), trifluoromethanesulfonic acid (19.1 g, 2.00 Eq, 127 mmol), 2,2,2-trifluoroacetic anhydride (16.1 g, 1.20 Eq, 76.7 mmol) and DCM (120 mL). The reaction mixture was stirred at 25° C. for 2 hours. The mixture was diluted with 100 mL of ice water, extracted with EtOAc (150 mL×3), and the combined organic layers were washed with water (100 mL×2), brine (50 mL), dried over anhydrous Na₂SO₄ and concentrated under reduced pressure. The crude product was purified by MPLC with the following conditions: Silica gel column 330 g, PE/EtOAc system, the ratio of EtOAc from 0% to 85% in 15 min, Flow rate: 40 mL/min; Wave Length: 254 nm. The collected fractions were concentrated under reduced pressure to provide 7-methoxychroman-4-one (7.7 g, 39 mmol, 61%, 90% Purity) as a yellow oil. MS: Calc'd for C₁₀H₁₀O₃:178.06, found [M+H]⁺: 179.2, 181.2.

Step 3: Into a 250-mL round bottom flask, was placed a mixture of 7-methoxychroman-4-one (8.0 g, 1 Eq, 45 mmol), silicon dioxide (800 mg, 0.30 Eq, 13.3 mmol), Et₂O(96 mL) and MeCN (32 mL). To the above mixture was added 1-bromopyrrolidine-2,5-dione (9.60 g, 1.2 Eq, 53.9 mmol). The reaction mixture was stirred at 25° C. for 3.5 hour. The crude product was purified by MPLC with the following conditions: Silica gel column 120 g, PE/EtOAc system, the ratio of EtOAc from 0% to 85% in 15 min, Flow rate: 80 mL/min; Wave Length: 254 nm. The collected fractions were concentrated under reduced pressure to provide 6-bromo-7-methoxychroman-4-one (9.5 g, 37 mmol, 82%) as a light yellow solid. MS: Calc'd for C₁₀H₉BrO₃:255.97, found [M+H]: 257.0, 259.0.

Step 4: Into a 500-mL 3-neck flask, under an inert atmosphere of nitrogen, was placed 6-bromo-7-methoxychroman-4-one (9.5 g, 1 Eq, 37 mmol) in THF (190 mL) at 25° C. The mixture was cooled to −78° C., then lithium diisopropylamide in THF (4.7 g, 22 mL, 2 molar, 1.2 Eq, 44 mmol)_{w} as added at −78° C. The mixture was stirred for 1 hour at −78° C. Then, diethyl oxalate (8.1 g, 1.5 Eq, 55 mmol) in THF (8 ml)_{w} as added dropwise to the reaction mixture at −78° C. The resulting mixture was stirred for 1 hour at 0° C. The resulting mixture was quenched by addition of a saturated solution of NH₄Cl (100 ml) and adjusted to pH=6 by the addition of saturated NaHSO₄. The solution was extracted with EtOAc (100 mL×3), and the combined organic layers were washed with brine (200 mL×2). A lot of solid precipitated in the organic layers, which was filtered, and the cake was washed using EA, then dried in vacuo. The solid resulted in ethyl (Z)-2-(6-bromo-7-methoxy-4-oxochroman-3-ylidene)-2-hydroxyacetate (8.0 g, 22 mmol, 61%) as a yellow solid. MS: Calc'd for C₁₄H₁₃BrO6:355.99, found [M+H]: 356.9, 358.9.

Step 5: To a solution of ethyl (Z)-2-(6-bromo-7-methoxy-4-oxochroman-3-ylidene)-2-hydroxyacetate (9.3 g, 1 Eq, 26 mmol) in t-BuOH (20 mL) and AcOH (300 mL)_{w} as added (3,5-dichlorophenyl) hydrazine hydrochloride (5.0 g, 0.90 Eq, 23 mmol) at RT under N₂. The mixture was stirred at 100° C. for 4 hours. Then the mixture was concentrated in vacuo. The residue was dissolved into EtOAc (800 mL), washed with water (200 mL), brine (100 mL), and a saturated solution of anhydrous sodium sulfate, then concentrated in vacuo. The residue was triturated with 200 mL PE to afford ethyl 8-bromo-1-(3,5-dichlorophenyl)-7-methoxy-1,4-dihydrochromeno[4,3-c]pyrazole-3-carboxylate (8.0 g, 14 mmol, 56%, 90% Purity) as an off-white solid, which was used in the next step without further purification. MS: Calc'd for C₂₀H₁₅BrCl₂N₂O₄:495.96, found [M+H]: 497.0, 499.0.

Step 6: Into a 100 mL round bottom flask, under an inert atmosphere of nitrogen, was a mixture of ethyl 8-bromo-1-(3,5-dichlorophenyl)-7-methoxy-1,4-dihydrochromeno[4,3-c]pyrazole-3-carboxylate (2.55 g, 1 Eq, 5.12 mmol), 3-(4,4,5,5-tetramethyl-1,3,2-dioxaborolan-2-yl)aniline (1.1 g, 0.98 Eq, 5.0 mmol), 1,1′-Bis(di-t-butylphosphino) ferrocene palladium dichloride (137 mg, 0.0411 Eq, 210 μmol), K₂CO₃ (2.12 g, 3.00 Eq, 15.3 mmol), 1,4-Dioxane (77 mL) and water (7.7 mL). The reaction mixture was stirred at 60° C. for 3 hours. The resulting mixture was extracted with DCM (50 mL×3), dried over anhydrous Na₂SO₄, concentrated and purified by MPLC with the following conditions: Silica gel column 40 g, PE/EtOAc system, the ratio of EtOAc from 0% to 85% in 15 min, Flow rate: 40 mL/min; Wave Length: 254 nm. This resulted in ethyl 8-(3-aminophenyl)-1-(3,5-dichlorophenyl)-7-methoxy-1,4-dihydrochromeno[4,3-c]pyrazole-3-carboxylate (1.74 g, 3.1 mmol, 60%, 90% Purity) as a yellow solid. MS: Calc'd for C₂₆H₂₁C₁₂N304:509.09, found [M+H]: 510.1, 512.1.

Step 7: Into a 40-mL vial, was placed a mixture of TEA (327 mg, 450 μL, 3.00 Eq, 3.23 mmol) and ethyl 8-(3-aminophenyl)-1-(3,5-dichlorophenyl)-7-methoxy-1,4-dihydrochromeno[4,3-c]pyrazole-3-carboxylate in THF (6 mL). A solution of triphosgene (256 mg, 0.801 Eq, 863 μmol) in THF (1 ml)_{w} as added dropwise at 0° C. The reaction mixture was stirred at 0° C. for 1 hour. The resulting mixture was used directly in the next step without any purification. MS: Calc'd for C₂₇H₁₉C₁₂N305:535.07, found [M+H]: 536.2, 538.2.

Step 8: Into an 8-mL vial, was placed a mixture of ethyl 1-(3,5-dichlorophenyl)-8-(3-isocyanatophenyl)-7-methoxy-1,4-dihydrochromeno[4,3-c]pyrazole-3-carboxylate (1 Eq) in THF (3 mL), then tert-butyl(20-amino-3,6,9, 12,15, 18-hexaoxaicosyl) carbamate (2 Eq)_{w} as added. The reaction mixture was stirred at 25° C. for 1 hour. The mixture was concentrated under reduced pressure. The mixture was directly purified by Prep-HPLC with the following conditions: Column, WelFlash™, C18 120 g, Spherical 20-40 μm; Mobile phase, Water (0.05% FA) and ACN (5% ACN to 5% ACN in 1 min, 5% ACN up to 98% in 6 min, 98% ACN to 98% in 2 min); Total flow rate, 70 mL/min; Detector, UV 220 nm. The collected fractions were dried by lyophilization to provide ethyl 1-(3,5-dichlorophenyl)-8-(3-(3-(2,2-dimethyl-4-oxo-3,8,11, 14, 17,20,23-heptaoxa-5-azapentacosan-25-yl)ureido)phenyl)-7-methoxy-1,4-dihydrochromeno[4,3-c]pyrazole-3-carboxylate (610 mg, 0.57 mmol, 90% Purity) as a colorless oil. MS: Calc'd for C₄₆H₅₉C₁₂N₅O₁₃:959.35, found [M+H]⁺: 960.2, 962.0.

Step 9: Into a 40-mL vial, was placed a mixture of ethyl 1-(3,5-dichlorophenyl)-8-(3-(3-(2,2-dimethyl-4-oxo-3,8,11,14,17,20,23-heptaoxa-5-azapentacosan-25-yl)ureido)phenyl)-7-methoxy-1,4-dihydrochromeno[4,3-c]pyrazole-3-carboxylate (610 mg, 1 Eq, 635 μmol), LiOH (152 mg, 10.0 Eq, 6.35 mmol), THF (6 mL) and water (2 mL). The reaction mixture was stirred at 25° C. for 5 hours. The reaction mixture was concentrated under reduced pressure to remove most of the THF, the residue was diluted with water (50 mL), then the pH value was adjusted to 6.0 by addition of a saturated NaHSO₄ solution. Filtration of the formed solid afforded 1-(3,5-dichlorophenyl)-8-(3-(3-(2,2-dimethyl-4-oxo-3,8,11,14, 17,20,23-heptaoxa-5-azapentacosan-25-yl)ureido)phenyl)-7-methoxy-1,4-dihydrochromeno[4,3-c]pyrazole-3-carboxylic acid (380 mg, 0.37 mmol, 58%, 90% Purity) as a light yellow solid, which was used directly in the next step without any purification. MS: Calc'd for C₄H₅₅C₁₂N₅O₁₃:931.32, found [M+H]⁺: 932.5, 934.5.

Step 10: Into a 40-mL vial, was placed a mixture of 1-(3,5-dichlorophenyl)-8-(3-(3-(2,2-dimethyl-4-oxo-3,8, 11,14, 17,20,23-heptaoxa-5-azapentacosan-25-yl)ureido)phenyl)-7-methoxy-1,4-dihydrochromeno[4,3-c]pyrazole-3-carboxylic acid (380 mg, 1 Eq, 407 μmol), HATU (186 mg, 1.20 Eq, 489 μmol), DIEA (158 mg, 213 μL, 3.00 Eq, 1.22 mmol) and DMF (4 mL). The reaction mixture was stirred at 25° C. for 10 minutes, then N,2-dimethylpropan-2-amine (71 mg, 2.0 Eq, 0.81 mmol)_{w} as added and the reaction mixture was stirred at 25° C. for an additional 1 hour. The mixture was directly purified by Prep-HPLC with the following conditions: Column, WelFlash™, C18 120 g, Spherical 20-40 μm; Mobile phase, Water (0.05% FA) and ACN (5% ACN to 5% ACN in 1 min, 5% ACN up to 98% in 6 min, 98% ACN to 98% in 2 min); Total flow rate, 70 mL/min; Detector, UV 220 nm. The collected fractions were dried by lyophilization to provide tert-butyl(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8,11,14,17,20-hexaoxa-2-azadocosan-22-yl) carbamate (260 mg, 0.23 mmol, 57%, 90% Purity) as a colorless oil. MS: Calc'd for C₄₉H₆₆C₁₂N6012:1000. 41, found [M+H]⁺: 1001.3, 1003.3.

Step 11: Into an 8-mL vial, was placed a mixture of tert-butyl(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8,11,14,17,20-hexaoxa-2-azadocosan-22-yl) carbamate (250 mg, 1 Eq, 250 μmol) and DCM (5 mL), to which was added zinc bromide (281 mg, 66.5 uL, 5.00 Eq, 1.25 mmol). The reaction mixture was stirred at 25° C. for 1.5 hour. The resulting mixture was concentrated, DMF was added (1 ml), then the mixture was purified by Prep-PLC with the following conditions: Column, WelFlash™, C18 120 g, Spherical 20-40 μm; Mobile phase, water (0.1% FA) and ACN (5% ACN to 5% ACN in 1 min, 10% ACN up to 98% in 6 min, 98% ACN to 98% in 1 min); Total flow rate, 70 mL/min; Detector, UV 220 nm. This resulted in 8-(3-(3-(20-amino-3,6,9,12, 15,18-hexaoxaicosyl)ureido)phenyl)-N-(tert-butyl)-1-(3,5-dichlorophenyl)-7-methoxy-N-methyl-1,4-dihydrochromeno[4,3-c]pyrazole-3-carboxamide 2,2,2-trifluoroacetate (250 mg, 0.22 mmol, 89%, 90% Purity) as colorless oil. MS: Calc'd for C₄H₅₈C₁₂N6010:900.36, found [M+H]: 901.4, 903.4.

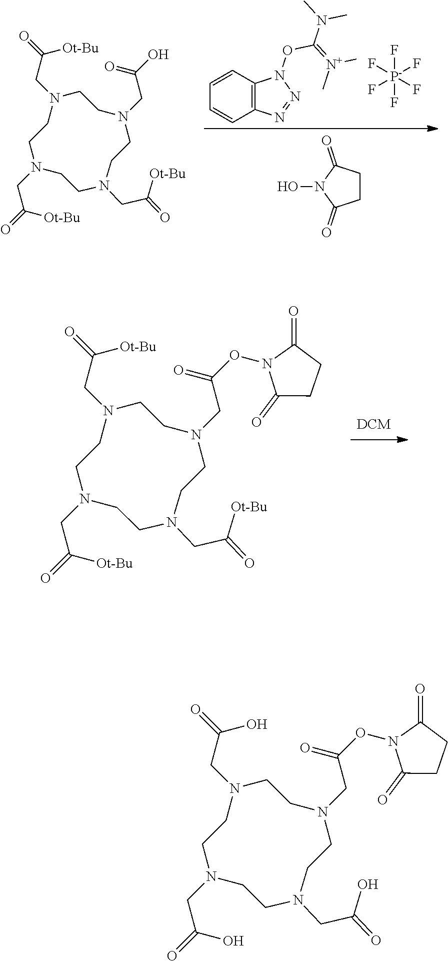 [SMILES: CC(C)(C)OC(=O)CN1CCN(CC(=O)O)CCN(CC(=O)OC(C)(C)C)CCN(CC(=O)OC(C)(C)C)CC1 ; CN(C)C(On1nnc2ccccc21)=[N+](C)C ; O=C1CCC(=O)N1O ; F[P-](F)(F)(F)(F)F ; CC(C)(C)OC(=O)CN1CCN(CC(=O)ON2C(=O)CCC2=O)CCN(CC(=O)OC(C)(C)C)CCN(CC(=O)OC(C)(C)C)CC1 ; O=C(O)CN1CCN(CC(=O)O)CCN(CC(=O)ON2C(=O)CCC2=O)CCN(CC(=O)O)CC1]

Step 12: Into a 40-mL vial, was placed a mixture of 2-(4,7,10-tris(2-(tert-butoxy)-2-oxoethyl)-1,4,7,10-tetraazacyclododecan-1-yl)acetic acid (5.0 g, 1 Eq, 8.7 mmol), 2-(1H-benzo[d][1,2,3]triazol-1-yl)-1,1,3,3-tetramethylisouronium hexafluorophosphate (V) (3.3 g, 1.0 Eq, 8.7 mmol) and ACN (20 mL). The reaction mixture was stirred at 25° C. for 60 minutes, then 1-hydroxypyrrolidine-2,5-dione (1.0 g, 1.0 Eq, 8.7 mmol)_{w} as added and the reaction mixture was stirred at 25° C. for an additional 12 hours. The mixture was concentrated under reduced pressure. The crude product was purified by MPLC with the following conditions: C18 120 g, ACN/H₂O system, the ratio of ACN from 0% to 85% in 20 min, Flow rate: 70 mL/min; Wave Length: 254 nm. The collected fractions were concentrated under reduced pressure to provide tri-tert-butyl 2,2′,2″-(10-(2-((2,5-dioxopyrrolidin-1-yl)oxy)-2-oxoethyl)-1,4,7,10-tetraazacyclododecane-1,4,7-triyl)triacetate (5.0 g, 4.5 mmol, 51%, 60% Purity) as a white solid. Calc'd for C₃₂H₅₅N₅O₁₀:669.39, found [M+H]⁺: 670.3.

Step 13: Into a 50-mL round-bottom flask, was placed a mixture of tri-tert-butyl 2,2′,2″-(10-(2-((2,5-dioxopyrrolidin-1-yl)oxy)-2-oxoethyl)-1,4,7,10-tetraazacyclododecane-1,4,7-triyl)triacetate (5.0 g, 1 Eq, 7.5 mmol) and DCM (20 mL), to which was added TFA (10 mL). The reaction mixture was stirred at 25° C. for 4 hours. The mixture was concentrated under reduced pressure. The crude product was precipitated using diethyl ether to provide 2,2′,2″-(10-(2-((2,5-dioxopyrrolidin-1-yl)oxy)-2-oxoethyl)-1,4,7,10-tetraazacyclododecane-1,4,7-triyl)triacetic acid (4.0 g, 6.8 mmol, 85% purity, 91% yield) as a white solid. Calc'd for C₂₀H₃₁N₅O₁₀:501.21, found [M+H]⁺: 502.4.

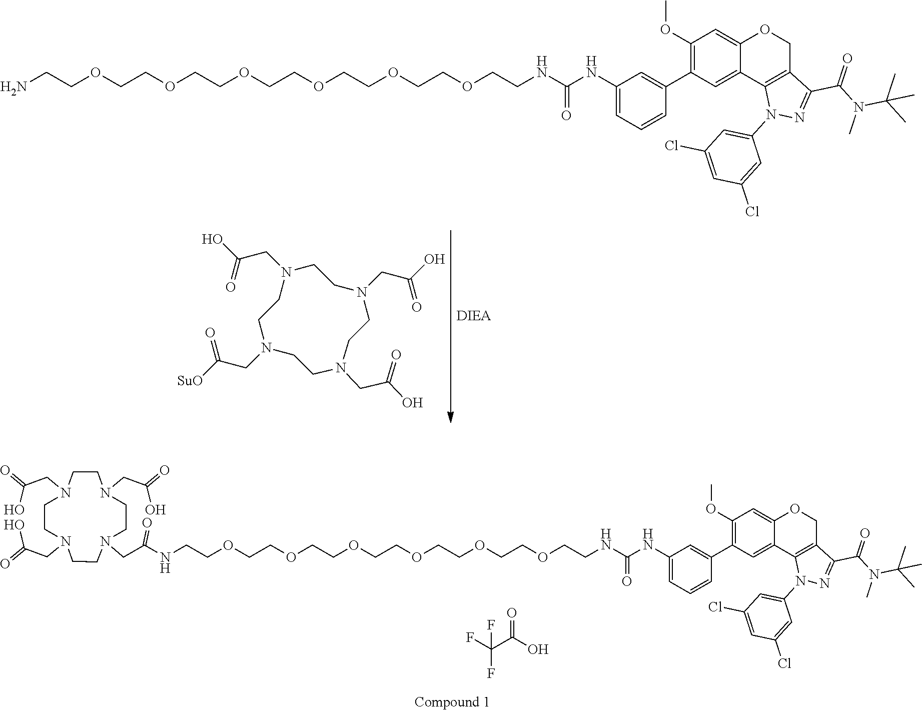

Step 14: Into a 25-mL round-bottom flask, was placed a mixture of 8-(3-(3-(20-amino-3,6,9,12,15, 18-hexaoxaicosyl)ureido)phenyl)-N-(tert-butyl)-1-(3,5-dichlorophenyl)-7-methoxy-N-methyl-1,4-dihydrochromeno[4,3-c]pyrazole-3-carboxamide (250 mg, 1 Eq, 277 μmol) in DMF (3 mL), then DIEA (180 mg, 243 μL, 5.02 Eq, 1.39 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (278 mg, 2.00 Eq, 554 μmol) were added. The resulting mixture was stirred at 25° C. for 1 hour. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8,11,14,17,20-hexaoxa-2,23-diazapentacosan-25-yl)-1,4,7,10-tetraazacyclododecane-1,4,7-triyl)triacetic acid--2,2,2-trifluoroacetic acid (160 mg, 0.11 mmol, 39% yield, 95% Purity) as a white solid. Calc'd for C₆₂H₈₅C₁₂F₃O₁₉:1286.54, found [M+H]⁺: 1287.5, 1289.5.

### Example 2:2,2′,2″-(10-(1-((3-(3-(tert-butyl(methyl) carbamoyl)-1-(3,5-dichlorophenyl)-7-methoxy-1,4-dihydrochromeno|4,3-c|pyrazol-8-yl)phenyl)amino)-1,18-dioxo-5,8,11,14-tetraoxa-2,17-diazanonadecan-19-yl)-1,4,7,10-tetraazacyclododecane-1,4,7-triyl)triacetic acid (Compound 2)

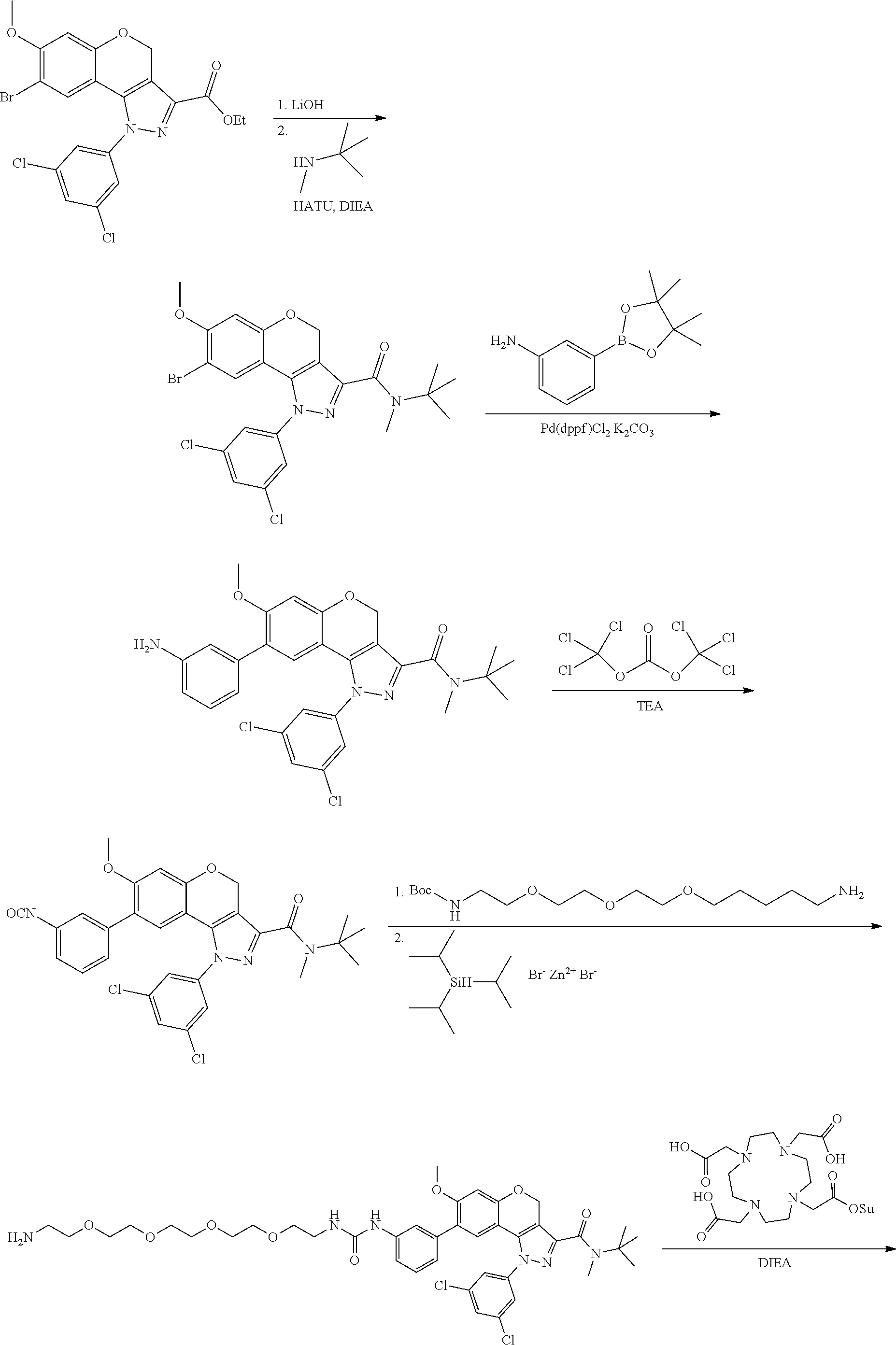

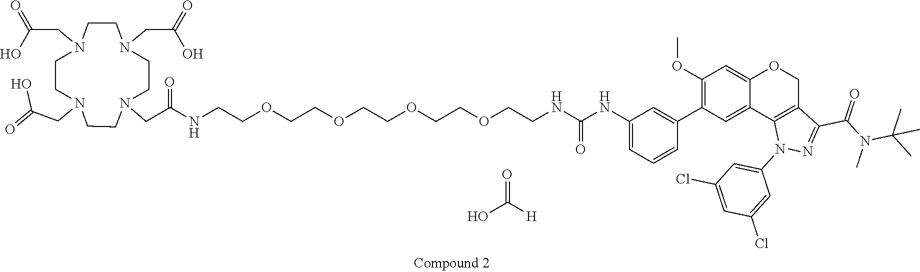 [SMILES: COc1cc2c(cc1-c1cccc(NC(=O)NCCOCCOCCOCCOCCNC(=O)CN3CCN(CC(=O)O)CCN(CC(=O)O)CCN(CC(=O)O)CC3)c1)-c1c(c(C(=O)N(C)C(C)(C)C)nn1-c1cc(Cl)cc(Cl)c1)CO2 ; O=CO]

Step 1: Into a 50-mL round-bottom flask, was placed ethyl 8-bromo-1-(3,5-dichlorophenyl)-7-methoxy-1,4-dihydrochromeno[4,3-c]pyrazole-3-carboxylate (2.0 g, 1 Eq, 4.0 mmol), EtOH (30 mL), LiOH (960 mg, 10 Eq, 40.1 mmol) and H₂O(10 mL). The reaction mixture was stirred at 20° C. for 16 hours. The reaction mixture was concentrated under reduced pressure to remove most of the EtOH, then the residue was diluted with water (30 mL), the pH value was adjusted to 6.0 by addition of a saturated NaHSO₄ solution. The precipitated solids were collected by filtration and washed with EA to provide 8-bromo-1-(3,5-dichlorophenyl)-7-methoxy-1,4-dihydrochromeno[4,3-c]pyrazole-3-carboxylic acid (1.8 g, 3.8 mmol, 95%) as a white solid. Calc'd for C₁₈H₁₁BrCl₂N₂O₄:467.93, found [M+H]⁺: 469.0, 471.0.

Step 2: Into a 100-mL round-bottom flask, was placed 8-bromo-1-(3,5-dichlorophenyl)-7-methoxy-1,4-dihydrochromeno[4,3-c]pyrazole-3-carboxylic acid (1.8 g, 1 Eq, 3.8 mmol), HATU (1.8 g, 1.2 Eq, 4.7 mmol), and DMF (20 mL). The reaction mixture was stirred at 20° C. for 10 minutes, then N,2-dimethylpropan-2-amine (0.55 g, 1.6 Eq, 6.3 mmol)_{w} as added and the reaction mixture was stirred at 20° C. for additional 2 hours. The mixture was diluted with 50 mL of water, extracted with EtOAc (50 mL×3), then the combined organic layers were washed with water (50 mL×2) and brine (50 mL), dried over anhydrous Na₂SO₄ and concentrated under reduced pressure. The crude product was purified by MPLC with the following conditions: Silica gel column 40 g, PE/EtOAc system, the ratio of EtOAc from 0% to 85% in 15 min, Flow rate: 50 mL/min; Wave Length: 254 nm. The collected fractions were concentrated under reduced pressure to provide 8-bromo-N-(tert-butyl)-1-(3,5-dichlorophenyl)-7-methoxy-N-methyl-1,4-dihydrochromeno[4,3-c]pyrazole-3-carboxamide (1.7 g, 3.2 mmol, 82%) as a white solid. Calc'd for C₂₃H₂₂BrCl₂N₃O₃:537.02, found [M+H]: 538.0, 540.0.

Step 3: Into a 100-mL round-bottom flask, purged and maintained under an inert atmosphere of nitrogen, was placed 8-bromo-N-(tert-butyl)-1-(3,5-dichlorophenyl)-7-methoxy-N-methyl-1,4-dihydrochromeno[4,3-c]pyrazole-3-carboxamide (1.7 g, 1 Eq, 3.2 mmol), 3-(4,4,5,5-tetramethyl-1,3,2-dioxaborolan-2-yl)aniline (0.70 g, 1.0 Eq, 3.2 mmol), K₂CO₃ (1.32 g, 3.0 Eq, 9.55 mmol), 1,4-Dioxane (20 mL), H₂O (2 mL), and 1,1′-Bis(di-t-butylphosphino) ferrocene palladium dichloride (0.2 g, 0.1 Eq, 0.3 mmol). The reaction mixture was stirred at 100° C. for 4 hours, then the reaction mixture was concentrated under reduced pressure. The crude product was purified by MPLC with the following conditions: Silica gel column 80 g, PE/EtOAc system, the ratio of EtOAc from 0% to 85% in 25 min, Flow rate: 55 mL/min; Wave Length: 254 nm. The collected fractions were concentrated under reduced pressure to provide 8-(3-aminophenyl)-N-(tert-butyl)-1-(3,5-dichlorophenyl)-7-methoxy-N-methyl-1,4-dihydrochromeno[4,3-c]pyrazole-3-carboxamide (1.2 g, 2.2 mmol, 69%) as a light yellow solid. Calc'd for C₂₉H₂₈C₁₂NO₃:550.15, found [M+H]: 551.2, 553.2.

Step 4: Into a 40-mL vial, was placed bis(trichloromethyl) carbonate (250 mg, 0.801 Eq, 843 μmol), THF (6 mL) and triethylamine (480 mg, 4.51 Eq, 4.74 mmol), to which was added the solution of 8-(3-aminophenyl)-N-(tert-butyl)-1-(3,5-dichlorophenyl)-7-methoxy-N-methyl-1,4-dihydrochromeno[4,3-c]pyrazole-3-carboxamide (580 mg, 1 Eq, 1.05 mmol) in THF (2 mL) dropwise at 0° C. The reaction mixture was stirred at 0° C. for 1 hour. There was 80% product in LCMS. The reaction solution was used to next step without purification. Calc'd for C₃₀H₂₆C₁₂N₄O₄:576.13, found [M+H]: 577.2, 579.2.

Step 5: Into a 40-mL vial, was placed N-(tert-butyl)-1-(3,5-dichlorophenyl)-8-(3-isocyanatophenyl)-7-methoxy-N-methyl-1,4-dihydrochromeno[4,3-c]pyrazole-3-carboxamide (600 mg, 1 Eq, 1.04 mmol) in THF (8 mL), to which was added a solution of tert-butyl(14-amino-3,6,9,12-tetraoxatetradecyl) carbamate (550 mg, 1.57 Eq, 1.63 mmol) in THF (2 mL) dropwise at 0° C. The reaction mixture was stirred at 20° C. for 1 hour. The crude mixture was concentrated under reduced pressure then purified by Prep-HPLC using the following conditions: Column, WelFlash™, C18 120 g, Spherical 20-40 μm; Mobile phase, Water (0.1% FA) and ACN (5% ACN to 5% ACN in 1 min, 25% ACN up to 90% in 8 min, 95% ACN to 95% in 3 min); Total flow rate, 70 mL/min; Detector, UV 220 nm. The collected fractions were concentrated under reduced pressure and dried by lyophilization to provide tert-butyl(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8,11,14-tetraoxa-2-azahexadecan-16-yl) carbamate (800 mg, 875 μmol, 84.2%) as a white solid. Calc'd for C₄₅H₅₈C₁₂N₆O₁₀:912.36, found [M+H]⁺: 913.2, 915.2.

Step 6: Into an 8-mL vial, was placed tert-butyl(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8, 11,14-tetraoxa-2-azahexadecan-16-yl) carbamate (800 mg, 1 Eq, 875 μmol), DCM (15 mL), tris(propan-2-yl) silane (800 mg, 1.04 mL, 5.77 Eq, 5.05 mmol), and zinc bromide (980 mg, 232 μL, 4.97 Eq, 4.35 mmol). The reaction mixture was stirred at 20° C. for 2 hours, then additional zinc bromide (980 mg, 232 μL, 4.97 Eq, 4.35 mmol)_{w} as added and the reaction mixture was stirred at 20° C. for an additional 2 hours. The mixture was concentrated under reduced pressure then 10 mL of DMF was added. The crude product was purified by Prep-HPLC with the following conditions: Column, WelFlash™, C18 120 g, Spherical 20-40 μm; Mobile phase, water (0.1% FA) and ACN (5% ACN to 5% ACN in 1 min, 25% ACN up to 80% in 8 min, 90% ACN to 90% in 3 min); Total flow rate, 70 mL/min; Detector, UV 220 nm. The collected fractions were concentrated under reduced pressure to remove most of the acetonitrile. The residue was diluted with water (20 mL), then the pH value was adjusted to 7.0 by addition of a saturated NaHCO₃ solution, then the product was extracted using EtOAc (50 mL×3). The combined organic layers were washed with brine (50 mL), dried over anhydrous Na₂SO₄ and concentrated under reduced pressure to provide 8-(3-(3-(14-amino-3,6,9,12-tetraoxatetradecyl)ureido)phenyl)-N-(tert-butyl)-1-(3,5-dichlorophenyl)-7-methoxy-N-methyl-1,4-dihydrochromeno[4,3-c]pyrazole-3-carboxamide (520 mg, 639 μmol, 73.0%) as a white solid. Calc'd for C₄₀H₅₀C₁₂N₆O₈:812.31, found [M+H]: 813.3, 815.3.

Step 7: Into an 8-mL vial, was placed 8-(3-(3-(14-amino-3,6,9,12-tetraoxatetradecyl)ureido)phenyl)-N-(tert-butyl)-1-(3,5-dichlorophenyl)-7-methoxy-N-methyl-1,4-dihydrochromeno[4,3-c]pyrazole-3-carboxamide (320 mg, 1 Eq, 393 μmol), DMF (3 mL), DIEA (250 mg, 337 μL, 4.92 Eq, 1.93 mmol), and 2,2′,2″-(10-(2-((2,5-dioxopyrrolidin-1-yl)oxy)-2-oxoethyl)-1,4,7,10-tetraazacyclododecane-1,4,7-triyl)triacetic acid (400 mg, 2.03 Eq, 798 μmol). The reaction mixture was stirred at 25° C. for 1 hour. The crude product was purified by Prep-HPLC with the following conditions: Column: SunFire prep OBD 19*150 mm 5 μm; Mobile Phase A: Water (0.1% FA); Mobile Phase B: ACN; Gradient: 20% B to 55% B in 10 min; Flow rate: 20 mL/min; Wave Length: 220 nm. The collected fractions were dried by lyophilization to provide 2,2′,2″-(10-(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1,18-dioxo-5,8,11, 14-tetraoxa-2, 17-diazanonadecan-19-yl)-1,4,7,10-tetraazacyclododecane-1,4,7-triyl)triacetic acid--formic acid (1/1) (220 mg, 177 μmol, 44.9%) as a white solid. Calc'd for C₄₀H₅₀C₁₂N₆O₈:1198.49, found [M+H−FA]⁺: 1199.8, 1201.8.

### Example 3:2,2′,2″-(10-(24-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-2,24-dioxo-6,9,12,15,18,21-hexaoxa-3-azatetracosyl)-1,4,7,10-tetraazacyclododecane-1,4,7-triyl)triacetic acid (Compound 3)

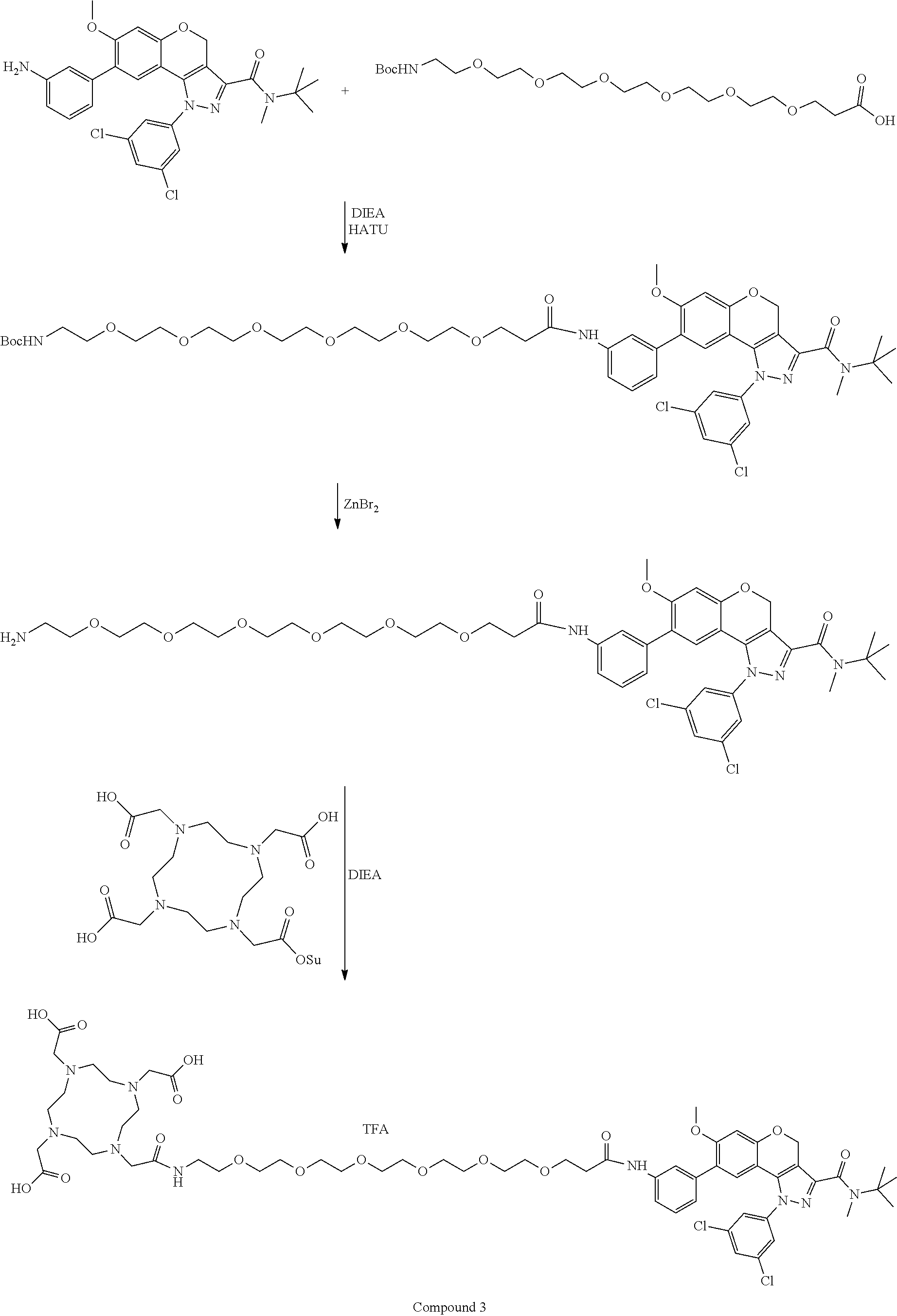

Step 1: Into a 40-mL vial, was placed a mixture of 2,2-dimethyl-4-oxo-3,8, 11, 14, 17,20,23-heptaoxa-5-azahexacosan-26-oic acid (200 mg, 1.52 Eq, 441 μmol), N-ethyl-N-isopropylpropan-2-amine (120 mg, 3.20 Eq, 928 μmol), 2-(3H-[1,2,3]triazolo[4,5-b]pyridin-3-yl)-1,1,3,3-tetramethylisouronium hexafluorophosphate (V) (150 mg, 1.36 Eq, 394 μmol), and DMF (1 mL). The reaction mixture was stirred at 20° C. for 10 minutes, then 8-(3-aminophenyl)-N-(tert-butyl)-1-(3,5-dichlorophenyl)-7-methoxy-N-methyl-1,4-dihydrochromeno[4,3-c]pyrazole-3-carboxamide (160 mg, 1 Eq, 290 μmol)_{w} as added and the reaction mixture was stirred at 25° C. for an additional 1 hour. The mixture was directly purified by MPLC with the following conditions: Column, WelFlash™, C18 120 g, Spherical 20-40 μm; Mobile phase, Water (0.05% TFA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide tert-butyl(21-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-21-oxo-3,6,9,12,15, 18-hexaoxahenicosyl) carbamate (180 mg, 182 μmol, 62.9%) as a yellow oil. Calc'd for C₄₉H₆₅Cl2N₅O₁₂:985.40, found [M+H]: 986.4, 988.3.

Step 2: Into a 50-mL round bottom flask, was placed a mixture of tert-butyl(21-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-21-oxo-3,6,9,12, 15, 18-hexaoxahenicosyl) carbamate (170 mg, 1 Eq, 172 μmol), zinc (II) bromide (400 mg, 10.3 Eq, 1.78 mmol), and DCM (4 mL). The reaction mixture was stirred at 25° C. for 16 hours then 5 ml of DMF was added. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm; to provide 8-(3-(1-amino-3,6,9,12, 15, 18-hexaoxahenicosan-21-amido)phenyl)-N-(tert-butyl)-1-(3,5-dichlorophenyl)-7-methoxy-N-methyl-1,4-dihydrochromeno[4,3-c]pyrazole-3-carboxamide (60 mg, 68 μmol, 39%) as a yellow oil. Calc'd for C₄₄H₅₇C₁₂N₅O₁₀:885.34, found [M+H]: 886.4, 888.4.

Step 3: Into a 2-mL vial, was placed a mixture of 8-(3-(1-amino-3,6,9,12, 15, 18-hexaoxahenicosan-21-amido)phenyl)-N-(tert-butyl)-1-(3,5-dichlorophenyl)-7-methoxy-N-methyl-1,4-dihydrochromeno[4,3-c]pyrazole-3-carboxamide (5 mg, 1 Eq, 6 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (5 mg, 2 Eq, 0.01 mmol), DIEA (3 mg, 4 μL, 4 Eq, 0.02 mmol) and DMF (0.01 mL). The reaction mixture was stirred at 60° C. for 1 hour. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide the product (9.9 mg, 7.1 μmol, 12%). Calc'd for C₆₂H₈₃C₁₂F3N₉O₁₈: 1368.52, found [M+H−TFA]⁺: 1272.5, 1274.5.

### Example 4:2.2′2″-(10-(1-3-(3-(ter-butyl(methyl) carbamoyl)-1-(3.5-dichlorophenyl)-7-methoxy-1.4-dihydrochromeno/4.3-clpyrazol-8-yl)phenyl)-1.24-dioxo-5.8.11.14.17.20-hexaoxa-2.23-diazapentacosan-25-yl)-1.4.7.10-tetraazacyclododecane-1.4.7-triyl)triacetic acid (Compound 4)

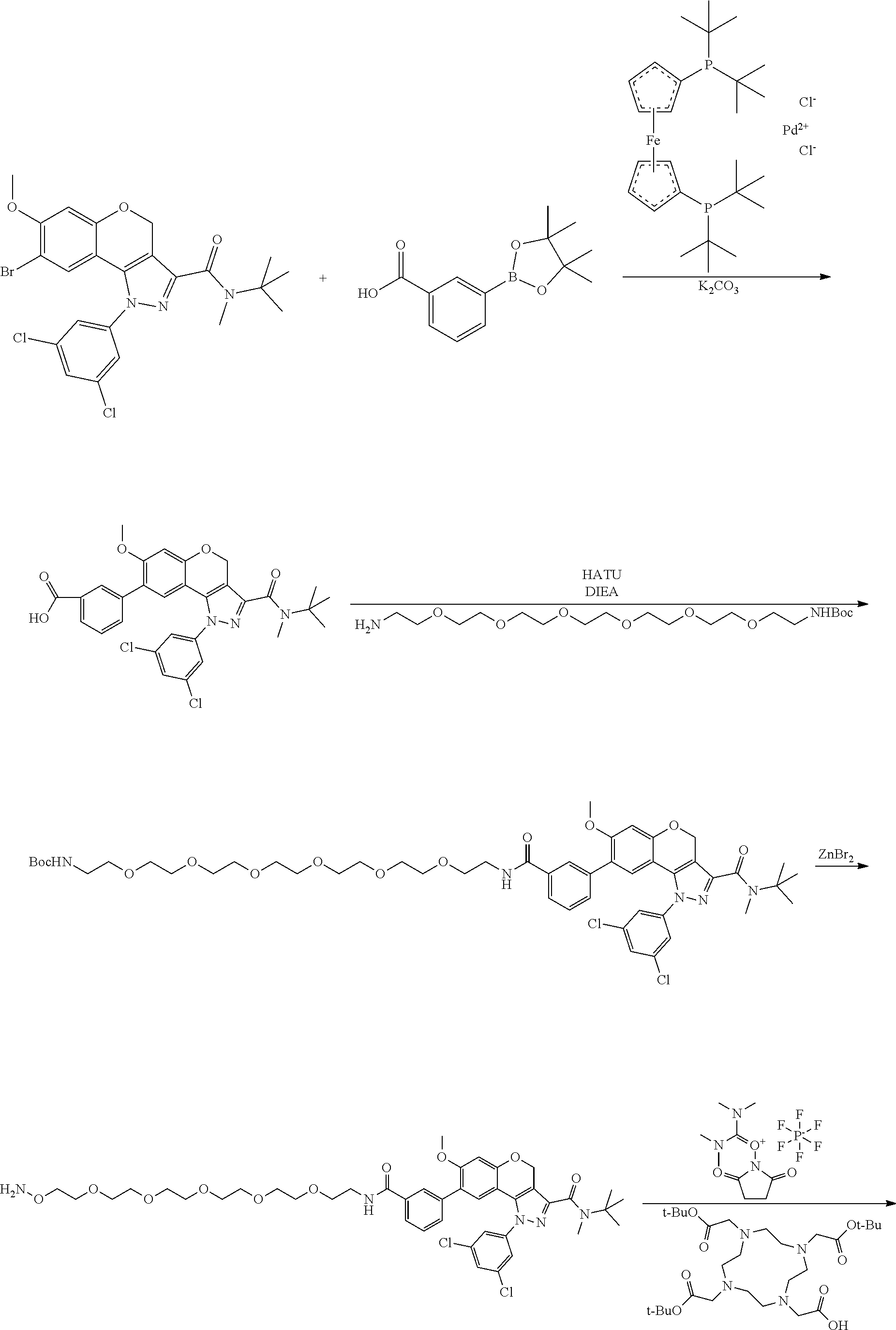

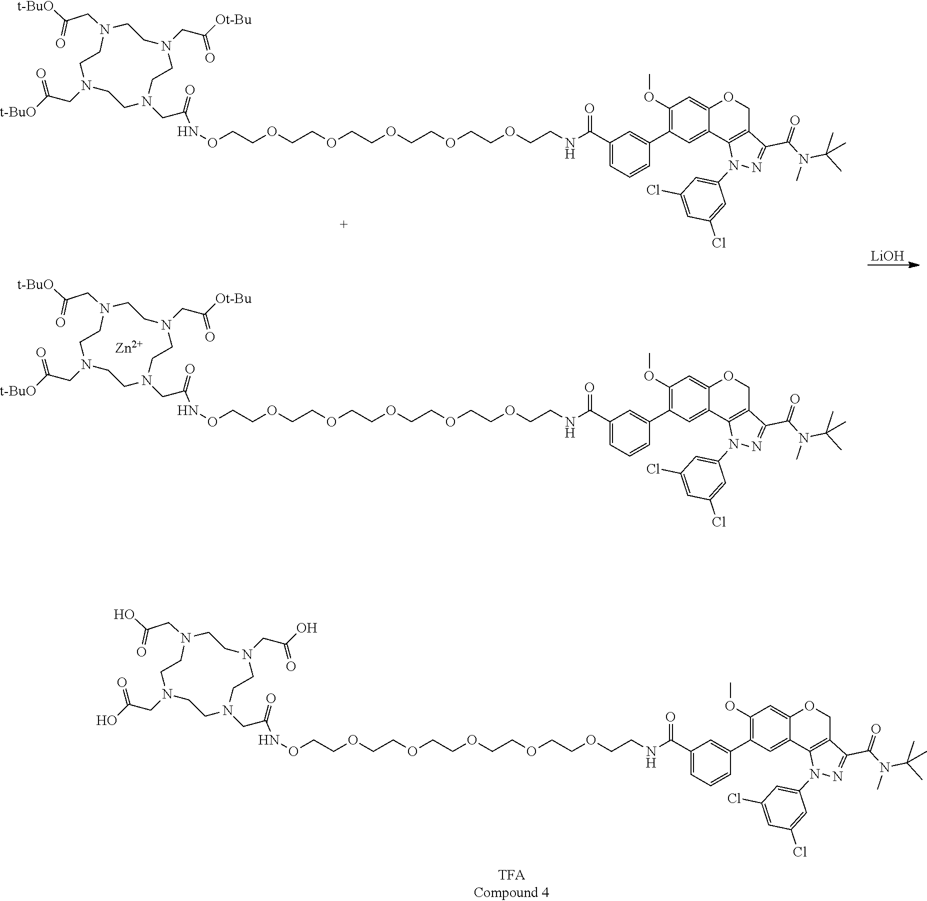

Step 1: Into a 40 mL vial, purged and maintained under an inert atmosphere of nitrogen, was placed a mixture of 8-bromo-N-(tert-butyl)-1-(3,5-dichlorophenyl)-7-methoxy-N-methyl-1,4-dihydrochromeno[4,3-c]pyrazole-3-carboxamide (from Example 2, Step 2; 350 mg, 1 Eq, 649 μmol), 3-(4,4,5,5-tetramethyl-1,3,2-dioxaborolan-2-yl)benzoic acid (160 mg, 0.994 Eq, 645 μmol), potassium carbonate (270 mg, 3.01 Eq, 1.95 mmol), 1,1′-Bis(di-t-butylphosphino) ferrocene palladium dichloride (20 mg, 1 Eq, 649 μmol), 1,4-dioxane (4 mL) and water (0.4 mL). The reaction mixture was stirred at 80° C. for 1 hour. The mixture was directly purified by MPLC using the following conditions: Column, WelFlash™, C18 120 g, Spherical 20-40 μm; Mobile phase, Water (0.05% TFA) and ACN (5% ACN to 5% ACN in 1 min, 30% ACN up to 98% in 6 min, 98% ACN to 98% in 3 min); Total flow rate, 70 mL/min; Detector, UV 220. Purification provided 3-(3-(tert-butyl(methyl) carbamoyl)-1-(3,5-dichlorophenyl)-7-methoxy-1,4-dihydrochromeno[4,3-c]pyrazol-8-yl)benzoic acid (212 mg, 365 μmol, 56.3%) as a yellow solid. Calc'd for C₃₀H₂₇C₁₂N₃O₅:579.13, found [M+H]⁺: 580.1, 582.1.

Step 2: Into an 8-mL vial, was placed a mixture of 3-(3-(tert-butyl(methyl) carbamoyl)-1-(3,5-dichlorophenyl)-7-methoxy-1,4-dihydrochromeno[4,3-c]pyrazol-8-yl)benzoic acid (200 mg, 1 Eq, 345 μmol), 2-(3H-[1,2,3]triazolo[4,5-b]pyridin-3-yl)-1,1,3,3-tetramethylisouronium hexafluorophosphate (V) (160 mg, 1.22 Eq, 421 μmol), DIEA (130 mg, 175 μL, 2.92 Eq, 1.01 mmol) and DMF (2 mL). The reaction mixture was stirred at 20° C. for 10 minutes, then tert-butyl (20-amino-3,6,9,12, 15, 18-hexaoxaicosyl) carbamate (200 mg, 1.37 Eq, 471 μmol)_{w} as added and the reaction mixture was stirred at 25° C. for an additional 1 hour. The mixture was directly purified by MPLC with the following conditions: Column, WelFlash™, C18 120 g, Spherical 20-40 μm; Mobile phase, Water (0.05% TFA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide tert-butyl(1-(3-(3-(tert-butyl(methyl) carbamoyl)-1-(3,5-dichlorophenyl)-7-methoxy-1,4-dihydrochromeno[4,3-c]pyrazol-8-yl)phenyl)-1-oxo-5,8, 11, 14, 17,20-hexaoxa-2-azadocosan-22-yl) carbamate (260 mg, 263 μmol, 76.5%) as a yellow oil. Calc'd for C₄₉H₆₅C₁₂N₅O₁₂:985.40, found [M+H]⁺: 986.3, 988.3.

Step 3: Into an 8-mL vial, was placed a mixture of tert-butyl(1-(3-(3-(tert-butyl(methyl) carbamoyl)-1-(3,5-dichlorophenyl)-7-methoxy-1,4-dihydrochromeno[4,3-c]pyrazol-8-yl)phenyl)-1-oxo-5,8,11, 14,17,20-hexaoxa-2-azadocosan-22-yl) carbamate (255 mg, 1 Eq, 258 μmol), zinc (II) bromide (580 mg, 9.97 Eq, 2.58 mmol), and DCM (5 mL). The reaction mixture was stirred at 25° C. for 5 hours. The reaction mixture was concentrated under reduced pressure to remove most of the DCM, then ACN (3 ml)_{w} as added. Solid ZnBr₂ was removed by filtration then the remaining residue was diluted with water (50 mL), extracted with DCM (50 mL×7), dried over anhydrous Na₂SO₄, and concentrated under reduced pressure to afford 8-(3-((20-amino-3,6,9, 12, 15, 18-hexaoxaicosyl) carbamoyl)phenyl)-N-(tert-butyl)-1-(3,5-dichlorophenyl)-7-methoxy-N-methyl-1,4-dihydrochromeno[4,3-c]pyrazole-3-carboxamide (180 mg, 203 μmol, 78.6%) as a light yellow solid, which was used directly in the next step without any purification. Calc'd for C₄₄H₅₇C₁₂N₅O₁₀:885.35, found [M+H]: 886.3, 888.3.

Step 4: Into an 8-mL vial, was placed a mixture of 2-(4,7,10-tris(2-(tert-butoxy)-2-oxoethyl)-1,4,7,10-tetraazacyclododecan-1-yl)acetic acid (170 mg, 1.55 Eq, 297 μmol), DIEA (80 mg, 0.11 mL, 3.2 Eq, 0.62 mmol), 2-(2,5-dioxopyrrolidin-1-yl)-1,1,3,3-tetramethyluronium hexafluorophosphate (V) (90 mg, 1.3 Eq, 0.25 mmol) and DMF (2 mL). The reaction mixture was stirred at 20° C. for 10 minutes, then 8-(3-((20-amino-3,6,9,12,15,18-hexaoxaicosyl) carbamoyl)phenyl)-N-(tert-butyl)-1-(3,5-dichlorophenyl)-7-methoxy-N-methyl-1,4-dihydrochromeno[4,3-c]pyrazole-3-carboxamide (170 mg, 1 Eq, 192 μmol)_{w} as added and the reaction mixture was stirred at 25° C. for an additional 1 hour. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tri-tert-butyl 2,2′,2″-(10-(1-(3-(3-(tert-butyl(methyl) carbamoyl)-1-(3,5-dichlorophenyl)-7-methoxy-1,4-dihydrochromeno[4,3-c]pyrazol-8-yl)phenyl)-1,24-dioxo-5,8,11,14, 17,20-hexaoxa-2,23-diazapentacosan-25-yl)-1,4,7,10-tetraazacyclododecane-1,4,7-triyl)triacetate, zinc (II) salt (80 mg, 53 μmol, 28%) as a light yellow solid. Product 2: Calc'd for C₇₂H₁₀₇C₁₂N₉O₁₇Zn₂:1503.64, found [(M+Zn2-)/2+H]: 753.2.

Step 5: Into an 8-mL vial, was placed a mixture of tri-tert-butyl 2,2′,2″-(10-(1-(3-(3-(tert-butyl(methyl) carbamoyl)-1-(3,5-dichlorophenyl)-7-methoxy-1,4-dihydrochromeno[4,3-c]pyrazol-8-yl)phenyl)-1,24-dioxo-5,8,11,14,17,20-hexaoxa-2,23-diazapentacosan-25-yl)-1,4,7,10-tetraazacyclododecane-1,4,7-triyl)triacetate, zinc (II) salt (70 mg, 1 Eq, 46 μmol), lithium hydroxide (33 mg, 30 Eq, 1.4 mmol), MeOH (0.3 mL) and water (0.3 mL). The reaction mixture was stirred at 80° C. for 4 hours. The reaction mixture was concentrated under reduced pressure to remove most of the MeOH, then the residue was diluted with water (50 mL). The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(1-(3-(3-(tert-butyl(methyl) carbamoyl)-1-(3,5-dichlorophenyl)-7-methoxy-1,4-dihydrochromeno[4,3-c]pyrazol-8-yl)phenyl)-1,24-dioxo-5,8,11,14, 17,20-hexaoxa-2,23-diazapentacosan-25-yl)-1,4,7,10-tetraazacyclododecane-1,4,7-triyl)triacetic acid--2,2,2-trifluoroacetic acid (1/1) (4.6 mg, 3.3 μmol, 7.1%) as a white solid. Calc'd for C₆₂H₈₄C₁₂F3N₉O₁₉:1385.52, found [(M+H−TFA]: 1272.7, 1274.7.

### Example 5:2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8,11,14,17,20-hexaoxa-2,23-diazapentacosan-25-yl)-1,4,7,10-tetraazacyclododecane-1,4,7-triyl)triacetic acid (Compound 5)

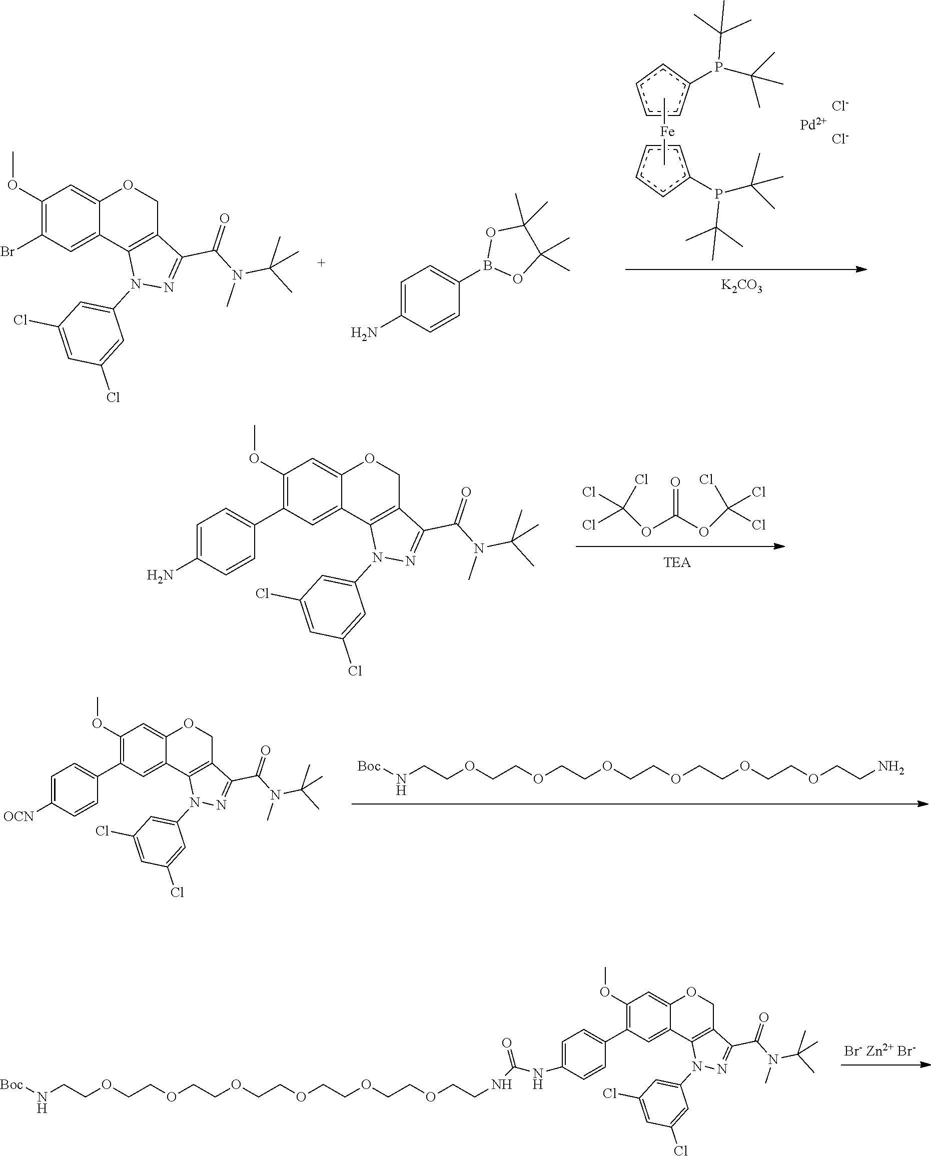

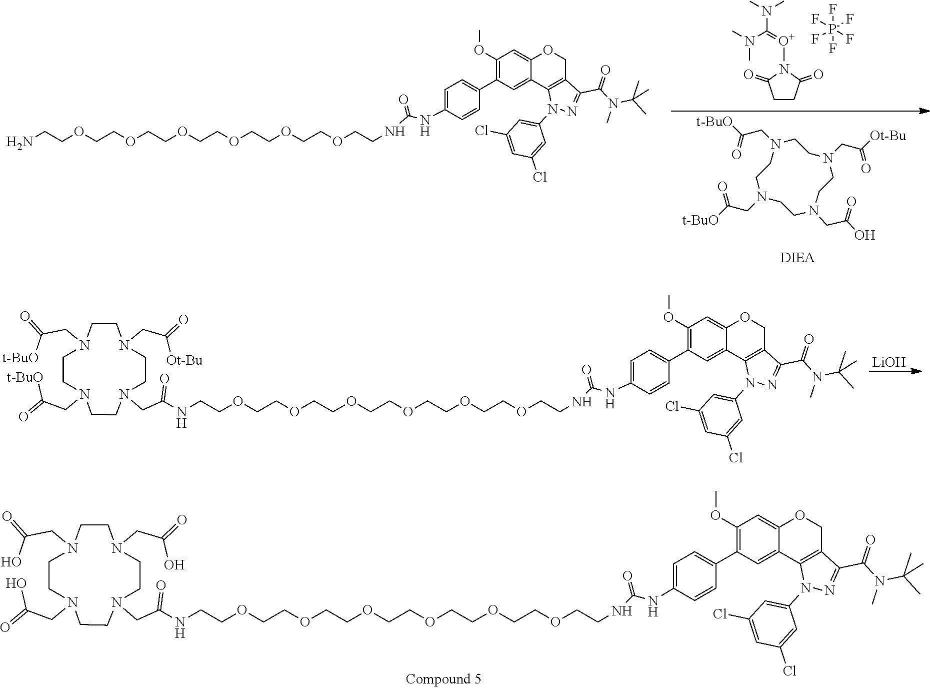

Step 1: Into a 40 mL vial, maintained under an inert atmosphere of nitrogen, was placed a mixture of 8-bromo-N-(tert-butyl)-1-(3,5-dichlorophenyl)-7-methoxy-N-methyl-1,4-dihydrochromeno[4,3-c]pyrazole-3-carboxamide (from Example 2, Step 2; 700 mg, 1 Eq, 1.30 mmol), 4-(4,4,5,5-tetramethyl-1,3,2-dioxaborolan-2-yl)aniline (256 mg, 0.900 Eq, 1.17 mmol), K₂CO₃ (538 mg, 3.00 Eq, 3.89 mmol), 1,1′-Bis(di-t-butylphosphino) ferrocene palladium dichloride (85 mg, 0.10 Eq, 0.13 mmol), 1,4-Dioxane (7 mL) and water (0.7 mL). The reaction mixture was stirred at 80° C. for 1 hour. The mixture was and concentrated and the crude product was purified by MPLC with the following conditions: Silica gel column 40 g, PE/EtOAc system, the ratio of EtOAc from 0% to 85% in 15 min, Flow rate: 40 mL/min; Wave Length: 254 nm. Purification provided 8-(4-aminophenyl)-N-(tert-butyl)-1-(3,5-dichlorophenyl)-7-methoxy-N-methyl-1,4-dihydrochromeno[4,3-c]pyrazole-3-carboxamide (600 mg, 1.09 mmol, 83.8%) as a yellow solid. Calc'd for C₂₉H₂₈C₁₂N₄O₃:550.15, found [M+H]⁺: 551.2, 553.2.

Step 2: Into a 40-mL vial, was placed a mixture of 8-(4-aminophenyl)-N-(tert-butyl)-1-(3,5-dichlorophenyl)-7-methoxy-N-methyl-1,4-dihydrochromeno[4,3-c]pyrazole-3-carboxamide (320 mg, 1 Eq, 580 μmol) and TEA (120 mg, 165 μL, 2.04 Eq, 1.19 mmol) in THF (3 mL), to which was added triphosgene (138 mg, 0.801 Eq, 465 μmol) in THF (1 ml) by dropwise addition at 0° C. The reaction mixture was stirred at 0° C. for 1 hour. The resulting mixture was used directly in the next step without any purification.

Step 3: Into an 8-mL vial, was placed a mixture of 8-(4-aminophenyl)-N-(tert-butyl)-1-(3,5-dichlorophenyl)-7-methoxy-N-methyl-1,4-dihydrochromeno[4,3-c]pyrazole-3-carboxamide (320 mg, 1 Eq, 580 μmol) and THF (1 mL), to which was added tert-butyl(20-amino-3,6,9, 12, 15, 18-hexaoxaicosyl) carbamate (176 mg, 1.50 Eq, 415 μmol). The reaction mixture was stirred at 25° C. for 1 hour. The mixture was concentrated under reduced pressure. The collected fractions were dried by lyophilization. This resulted to provide tert-butyl(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8, 11, 14, 17,20-hexaoxa-2-azadocosan-22-yl) carbamate (200 mg, 0.18 mmol, 65%, 90% Purity) as a colorless oil. Calc'd for C₄₉H₆₆C₁₂N₆O₁₂:1000.41, found [M+H]⁺: 1001.5, 1003.5.

Step 4: Into an 8-mL vial, was placed a mixture of tert-butyl(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8,11,14,17,20-hexaoxa-2-azadocosan-22-yl) carbamate (190 mg, 1 Eq, 190 μmol) in DCM (5 mL), to which was added zinc (II) bromide (427 mg, 10.0 Eq, 1.90 mmol). The reaction mixture was stirred at 25° C. for 1.5 hour. The resulting mixture was concentrated and MeCN was added, then the solution was sonicated for 2 min, then filtered and the filtrate was concentrated. The mixture was diluted with water (50 mL), extracted with EtOAc (50 mL×3), and the combined organic layers were washed with water (50 mL×2) and brine (50 mL), dried over anhydrous Na₂SO₄ and concentrated under reduced pressure to provide in 8-(4-(3-(20-amino-3,6,9,12, 15, 18-hexaoxaicosyl)ureido)phenyl)-N-(tert-butyl)-1-(3,5-dichlorophenyl)-7-methoxy-N-methyl-1,4-dihydrochromeno[4,3-c]pyrazole-3-carboxamide (160 mg, 177 μmol, 93.6%) as an off-white solid. Calc'd for C₄₄H₅₈C₁₂N₆O₁₀:900.36, found [M+H]⁺: 901.2, 903.2.

Step 5: Into an 8-mL vial, was placed a mixture of 2-(4,7,10-tris(2-(tert-butoxy)-2-oxoethyl)-1,4,7,10-tetraazacyclododecan-1-yl)acetic acid (80 mg, 0.84 Eq, 0.14 mmol) in DMF (0.5 mL), then N-ethyl-N-isopropylpropan-2-amine (160 mg, 7.44 Eq, 1.24 mmol) and 2-(2,5-dioxopyrrolidin-1-yl)-1,1,3,3-tetramethyluronium hexafluorophosphate (V) (48 mg, 0.80 Eq, 0.13 mmol)_{w} as added. The mixture was stirred at 25° C. for 10 mins. To the above mixture was added 8-(4-(3-(20-amino-3,6,9, 12, 15, 18-hexaoxaicosyl)ureido)phenyl)-N-(tert-butyl)-1-(3,5-dichlorophenyl)-7-methoxy-N-methyl-1,4-dihydrochromeno[4,3-c]pyrazole-3-carboxamide (160 mg, 177 μmol, 93.6%). The resulting mixture was stirred at 25° C. for 1 hour. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 5% ACN up to 98% in 6 min, 98% ACN to 98% in 3 min); Total flow rate, 70 mL/min; Detector, UV 220 nm. The collected fractions were dried by lyophilization to provide tri-tert-butyl 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8, 11, 14, 17,20-hexaoxa-2,23-diazapentacosan-25-yl)-1,4,7,10-tetraazacyclododecane-1,4,7-triyl)triacetate (150 mg, 93 μmol, 56%, 90% Purity) as a white solid. Calc'd for C₇₂H₁₀₈Cl₂N₁₀O₁₇:1454.73, found [M/2+H]: 728.3.

Step 6: Into an 8-mL vial, was placed a mixture of tri-tert-butyl 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8,11, 14, 17,20-hexaoxa-2,23-diazapentacosan-25-yl)-1,4,7,10-tetraazacyclododecane-1,4,7-triyl)triacetate (90 mg, 1 Eq, 62 μmol) in MeOH (2.4 mL) and H₂O(0.8 mL), then lithium hydroxide (30 mg, 20 Eq, 1.3 mmol)_{w} as added. The resulting mixture was stirred at 80° C. for 1 hour. The crude product was purified by Prep-HPLC with the following conditions: Column: SunFire prep OBD 19*150 mm 5 μm; Mobile Phase A: Water (0.1% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8, 11, 14, 17,20-hexaoxa-2,23-diazapentacosan-25-yl)-1,4,7,10-tetraazacyclododecane-1,4,7-triyl)triacetic acid--formic acid (1/1) (25.6 mg, 18 μmol, 29%, 95% Purity) as a white solid. Calc'd for C₆₁H₈₆C₁₂N₁₀O₁₉:1332. 54, found [M+H−FA]⁺: 1286.5, 1288.5.

### Example 6:2,2′,2″-(10-(1-((4-(3-(tert-butyl(methyl) carbamoyl)-1-(3,5-dichlorophenyl)-7-methoxy-1,4-dihydrochromeno|4,3-clpyrazol-8-yl)phenyl)amino)-1,18-dioxo-5,8,11,14-tetraoxa-2,17-diazanonadecan-19-yl)-1,4,7,10-tetraazacyclododecane-1,4,7-triyl)triacetic acid (Compound 6)

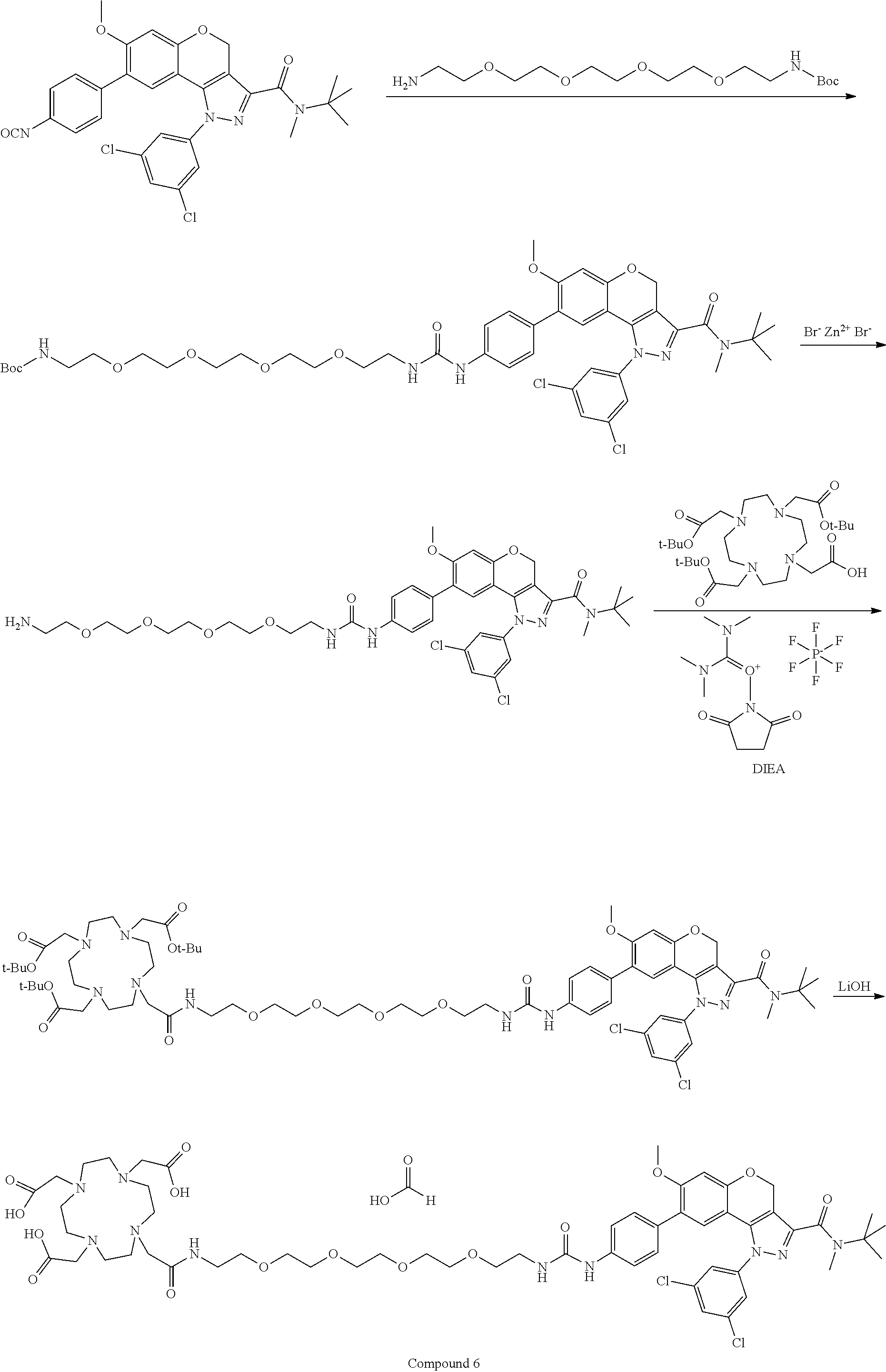

Step 1: Into an 8-mL vial, was placed a mixture of N-(tert-butyl)-1-(3,5-dichlorophenyl)-8-(4-isocyanatophenyl)-7-methoxy-N-methyl-1,4-dihydrochromeno[4,3-c]pyrazole-3-carboxamide (150 mg, 1 Eq, 260 μmol) and THF (1 mL), to which was added tert-butyl(14-amino-3,6,9,12-tetraoxatetradecyl) carbamate (175 mg, 2.00 Eq, 520 μmol). The reaction mixture was stirred at 25° C. for 1 hour. The mixture was concentrated under reduced pressure. The collected fractions were dried by lyophilization. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 5% ACN up to 98% in 6 min, 98% ACN to 98% in 2 min); Total flow rate, 70 mL/min; Detector, UV 220 nm. The collected fractions were dried by lyophilization to provide tert-butyl(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8, 11, 14-tetraoxa-2-azahexadecan-16-yl) carbamate (160 mg, 0.16 mmol, 61%, 90% Purity) as a white solid. Calc'd for C₄₅H₅₈C₁₂N₆O₁₀:912.36, found [M+H]: 913.4, 915.4.

Step 2: Into an 8-mL vial, was placed a mixture of tert-butyl(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8,11,14-tetraoxa-2-azahexadecan-16-yl) carbamate (160 mg, 1 Eq, 175 μmol) and DCM (2 mL), to which was added ZnBr2 (400 mg, 10.1 Eq, 1.78 mmol). The reaction mixture was stirred at 25° C. for 1.5 hours. The resulting mixture was concentrated and MeCN was added, the solution was sonicated for 2 min, filtered and the filtrate was concentrated. The mixture was diluted with water (50 mL) and extracted with EtOAc (50 mL×3), then the combined organic layers were washed with water (50 mL×2) and brine (50 mL), dried over anhydrous Na₂SO₄ and concentrated under reduced pressure to provide tert-butyl (1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1-oxo-5,8, 11, 14-tetraoxa-2-azahexadecan-16-yl) carbamate (160 mg, 0.16 mmol, 61%, 90% Purity) as a white solid. Calc'd for C₄₀H₅₀C₁₂N₆O₈:812.31, found [M+H]: 813.4, 815.4.

Step 3: Into an 8-mL vial, was placed a mixture of 2-(4,7,10-tris(2-(tert-butoxy)-2-oxoethyl)-1,4,7,10-tetraazacyclododecan-1-yl)acetic acid (100 mg, 1.09 Eq, 175 μmol) in DMF (2 mL), then N,N,N,N-tetramethyl-o-(n-succinimidyl)uronium hexafluorophosphate (70 mg, 1.2 Eq, 0.19 mmol) and DIEA (88 mg, 0.12 mL, 4.3 Eq, 0.68 mmol) were added. The mixture was stirred at 25° C. for 10 mins. To the above mixture was added 8-(4-(3-(14-amino-3,6,9,12-tetraoxatetradecyl)ureido)phenyl)-N-(tert-butyl)-1-(3,5-dichlorophenyl)-7-methoxy-N-methyl-1,4-dihydrochromeno[4,3-c]pyrazole-3-carboxamide (130 mg, 1 Eq, 160 μmol). The resulting mixture was stirred at 25° C. for 1 hour. The mixture was directly purified by MPLC using the following conditions: Column, WelFlash™, C18 120 g, Spherical 20-40 μm; Mobile phase, water (0.05% FA) and ACN (5% ACN to 5% ACN in 1 min, 5% ACN up to 98% in 6 min, 98% ACN to 98% in 3 min); Total flow rate, 70 mL/min; Detector, UV 220 nm. The collected fractions were dried by lyophilization to provide tri-tert-butyl 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,18-dioxo-5,8,11,14-tetraoxa-2, 17-diazanonadecan-19-yl)-1,4,7,10-tetraazacyclododecane-1,4,7-triyl)triacetate (130 mg, 85 μmol, 54%, 90% Purity) as a white solid. Calc'd for C₆₈H₁₀₀C₁₂N₁₀O₁₅:1366.67, found [M+H]⁺: 1367.3, 1369.3.

Step 4: Into an 8-mL vial, was placed a mixture of tri-tert-butyl 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,18-dioxo-5,8,11,14-tetraoxa-2,17-diazanonadecan-19-yl)-1,4,7,10-tetraazacyclododecane-1,4,7-triyl)triacetate (130 mg, 1 Eq, 95.0 μmol), MeOH (1.5 mL) and water (0.5 mL), then LiOH (23 mg, 10 Eq, 0.96 mmol)_{w} as added. The resulting mixture was stirred at 80° C. for 1 hour. The crude product was purified by Prep-HPLC using the following conditions: Column: SunFire prep OBD 19*150 mm 5 μm; Mobile Phase A: Water (0.1% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,18-dioxo-5,8,11, 14-tetraoxa-2, 17-diazanonadecan-19-yl)-1,4,7,10-tetraazacyclododecane-1,4,7-triyl)triacetic acid--formic acid (1/1) (22 mg, 17 μmol, 18%, 95% Purity) as a white solid. Calc'd for C₅₇H₇₈C₁₂N₁₀O₁₇:1244. 49, found [M+H−FA]⁺: 1199.6, 1201.6.

### Example 7:2,2′,2″-(10-(1-((3-(1-(3,5-dichlorophenyl)-3-(3,3-dimethylmorpholine-4-carbonyl)-7-methoxy-1,4-dihydrochromeno|4,3-c]pyrazol-8-yl)phenyl)amino)-1,24-dioxo-5,8,11,14,17,20-hexaoxa-2,23-diazapentacosan-25-yl)-1,4,7,10-tetraazacyclododecane-1,4,7-triyl)triacetic acid (Compound 7)

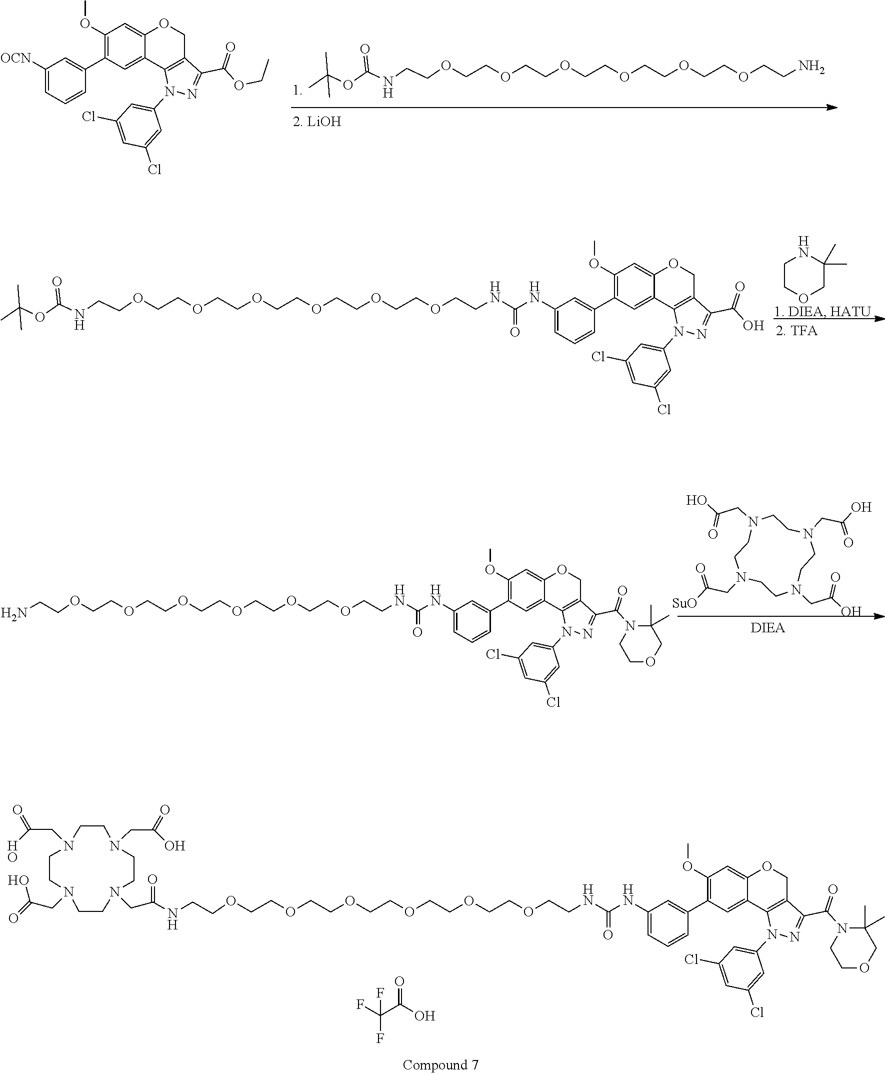

Step 1: Into a 40-mL vial, was placed a mixture of ethyl 1-(3,5-dichlorophenyl)-8-(3-isocyanatophenyl)-7-methoxy-1,4-dihydrochromeno[4,3-c]pyrazole-3-carboxylate (525 mg, 1 Eq, 979 μmol) and THF (5 mL), to which was added tert-butyl(20-amino-3,6,9,12,15,18-hexaoxaicosyl) carbamate (416 mg, 1.00 Eq, 980 μmol). The reaction mixture was stirred at 25° C. for 1 hour. The mixture was concentrated under reduced pressure. The mixture was directly purified by Prep-HPLC with the following conditions: Column, WelFlash™, C18 120 g, Spherical 20-40 μm; Mobile phase, Water (0.05% FA) and ACN (5% ACN to 5% ACN in 1 min, 5% ACN up to 98% in 6 min, 98% ACN to 98% in 2 min); Total flow rate, 70 mL/min; Detector, UV 220 nm. The collected fractions were dried by lyophilization to provide ethyl 1-(3,5-dichlorophenyl)-8-(3-(3-(2,2-dimethyl-4-oxo-3,8,11,14,17,20,23-heptaoxa-5-azapentacosan-25-yl)ureido)phenyl)-7-methoxy-1,4-dihydrochromeno[4,3-c]pyrazole-3-carboxylate (300 mg, 312 μmol, 31.9%) as a colorless oil. Calc'd for C₄₆H₅₉C₁₂N₅O₁₃:959.35, found [M+H]⁺: 960.2, 962.0.

Step 2: Into a 40-mL vial, was placed a mixture of ethyl 1-(3,5-dichlorophenyl)-8-(3-(3-(2,2-dimethyl-4-oxo-3,8,11,14,17,20,23-heptaoxa-5-azapentacosan-25-yl)ureido)phenyl)-7-methoxy-1,4-dihydrochromeno[4,3-c]pyrazole-3-carboxylate (300 mg, 1 Eq, 312 μmol), lithium hydroxide (23 mg, 3.1 Eq, 0.96 mmol), THF (1.5 mL) and water (0.3 mL). The reaction mixture was stirred at 25° C. for 1.5 hours. The reaction mixture was concentrated under reduced pressure to remove most of the THF, then the residue was diluted with water (1 mL). The pH value of the solution was adjusted to 6.0 by addition of a saturated NaHSO₄ solution. Precipitated solid was filtered to afford 1-(3,5-dichlorophenyl)-8-(3-(3-(2,2-dimethyl-4-oxo-3,8,11,14, 17,20,23-heptaoxa-5-azapentacosan-25-yl)ureido)phenyl)-7-methoxy-1,4-dihydrochromeno[4,3-c]pyrazole-3-carboxylic acid (200 mg, 0.19 mmol, 62%, 90% Purity) as a light yellow solid, which was used directly in the next step without any purification. Calc'd for C₄H₅₅C₁₂N₅O₁₃:931.32, found [M+H]⁺: 932.4, 934.4.

Step 3: Into an 8-mL vial, was placed a mixture of 1-(3,5-dichlorophenyl)-8-(3-(3-(2,2-dimethyl-4-oxo-3,8,11,14,17,20,23-heptaoxa-5-azapentacosan-25-yl)ureido)phenyl)-7-methoxy-1,4-dihydrochromeno[4,3-c]pyrazole-3-carboxylic acid (100 mg, 1 Eq, 107 μmol), 2-(3H-[1,2,3]triazolo[4,5-b]pyridin-3-yl)-1,1,3,3-tetramethylisouronium hexafluorophosphate (V) (50 mg, 1.2 Eq, 0.13 mmol), N-ethyl-N-isopropylpropan-2-amine (42 mg, 3.0 Eq, 0.32 mmol) and DMF (1 mL). The reaction mixture was stirred at 25° C. for 10 minutes, then 3,3-dimethylmorpholine (19 mg, 1.5 Eq, 0.16 mmol)_{w} as added and the reaction mixture was stirred at 25° C. for additional 1 hour. The mixture was directly purified by Prep-HPLC with the following conditions: Column, WelFlash™, C18 120 g, Spherical 20-40 μm; Mobile phase, Water (0.05% FA) and ACN (5% ACN to 5% ACN in 1 min, 5% ACN up to 98% in 6 min, 98% ACN to 98% in 2 min); Total flow rate, 70 mL/min; Detector, UV 220 nm. The collected fractions were concentrated to provide tert-butyl(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1-oxo-5,8,11, 14,17,20-hexaoxa-2-azadocosan-22-yl) carbamate (90 mg, 87 μmol, 82%) as a colorless oil. Calc'd for C₅₀H₆₆C₁₂N₆O₁₃:1028.41, found [M+H]⁺: 1029.4, 1031.4.

Step 4: Into an 8-mL vial, was placed a mixture of tert-butyl(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1-oxo-5,8, 11,14, 17,20-hexaoxa-2-azadocosan-22-yl) carbamate (82 mg, 1 Eq, 80 μmol), DCM (1 mL) and TFA (0.1 mL). The reaction mixture was stirred at 25° C. for 20 mins. The resulting mixture was concentrated and DMF (1 mL)_{w} as added, then the solution was purified by Prep-PLC with the following conditions: Column, WelFlash™, C18 120 g, Spherical 20-40 μm; Mobile phase, Water (0.1% FA) and ACN (5% ACN to 5% ACN in 1 min, 10% ACN up to 98% in 6 min, 98% ACN to 98% in 1 min); Total flow rate, 70 mL/min; Detector, UV 220 nm. Purification provided 1-(20-amino-3,6,9,12, 15, 18-hexaoxaicosyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (80 mg, 77 μmol, 97%, 90% Purity) as a colorless oil. Calc'd for C₄₅H₅₈C₁₂N₆O₁₁:928.35, found [M+H]⁺: 929.5, 931.5.

Step 5: Into a 25-mL round-bottom flask, was placed a mixture of 1-(20-amino-3,6,9,12,15, 18-hexaoxaicosyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (80 mg, 1 Eq, 86 μmol) in DMF (1 mL), then DIEA (56 mg, 75 μL, 5.0 Eq, 0.43 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (86 mg, 2.0 Eq, 0.17 mmol) were added. The resulting mixture was stirred at 25° C. for 1 hour. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,24-dioxo-5,8,11, 14, 17,20-hexaoxa-2,23-diazapentacosan-25-yl)-1,4,7,10-tetraazacyclododecane-1,4,7-triyl)triacetic acid--2,2,2-trifluoroacetic acid (1/1) (54.8 mg, 35.7 μmol, 42%, 93.3% Purity) as a white solid. Calc'd for C₆₃H₈₅C₁₂F3N₁₀O₂₀:1428.52, found [M+H−TFA]⁺: 1315.6, 1317.6.

### Example 8:2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,18-dioxo-5,8,11,14-tetraoxa-2,17-diazanonadecan-19-yl)-1,4,7,10-tetraazacyclododecane-1,4,7-triyl)triacetic acid (Compound 8)

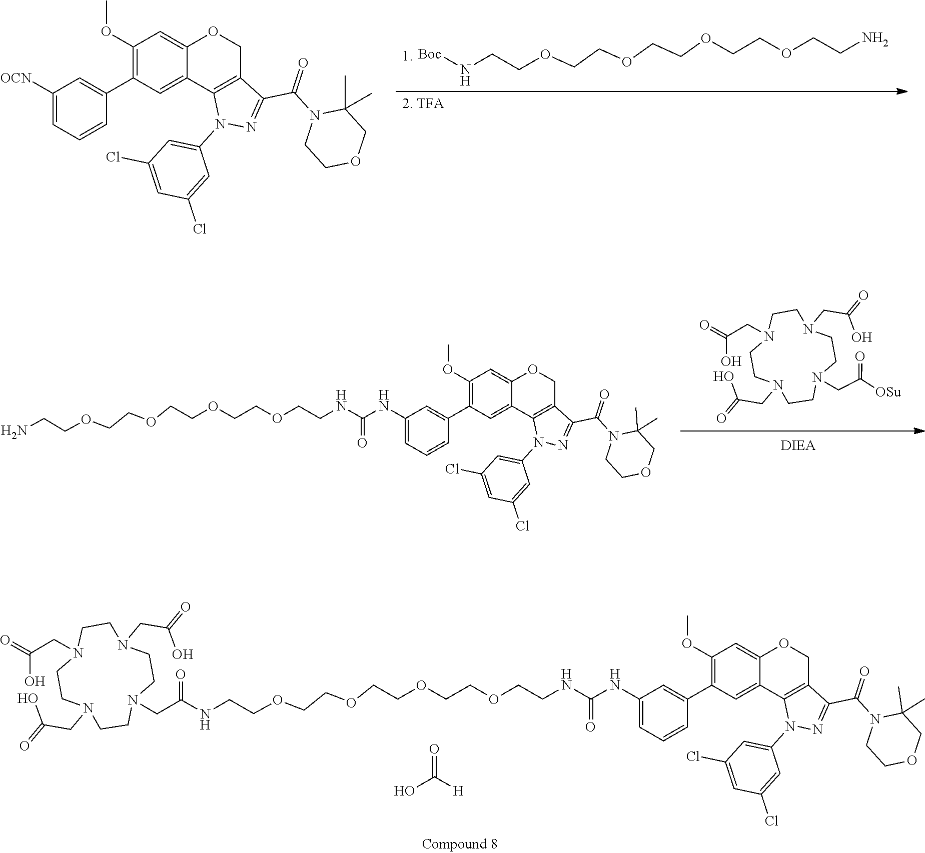

Step 1: Into a 40-mL vial, was placed a mixture of (1-(3,5-dichlorophenyl)-8-(3-isocyanatophenyl)-7-methoxy-1,4-dihydrochromeno[4,3-c]pyrazol-3-yl) (3,3-dimethylmorpholino) methanone (1 Eq) and THF (2.0 mL). The reaction mixture was stirred at 25° C. for 16 hours. The reaction mixture was filtered through a pad of celite, then the filtrate was concentrated and purified by MPLC using the following conditions: Column, C18 120 g; mobile phase, Water (0.1% FA) and ACN (30% ACN up to 98% in 8 min, 98% ACN to 98% in 3 min); Total flow rate, 70 mL/min; Detector, UV 220 nm. The collected fractions were concentrated under reduced pressure and dried to provide tert-butyl(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1-oxo-5,8,11,14-tetraoxa-2-azahexadecan-16-yl) carbamate (165 mg, 175 μmol) as a yellow oil. Calc'd for C₄₆H₅₈C₁₂N₆O₁₁:940.35, found [M+H]⁺: 941.4.

Step 2: Into an 8-mL vial, was placed a mixture of tert-butyl(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1-oxo-5,8, 11,14-tetraoxa-2-azahexadecan-16-yl) carbamate (155 mg, 1 Eq, 165 μmol) and DCM (1.0 mL), to which was added TFA (0.1 mL). The reaction mixture was stirred at 25° C. for 30 mins. The mixture was concentrated under reduced pressure to provide 1-(14-amino-3,6,9,12-tetraoxatetradecyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea 2,2,2-trifluoroacetate (76 mg, 80 μmol, 48%) as a yellow oil. Calc'd for C₄₃H₅₁C₁₂F₃N₆O₁₁:954.29, found [M+H−TFA]⁺: 841.2.

Step 3: Into an 8-mL vial, was placed a mixture of 1-(14-amino-3,6,9,12-tetraoxatetradecyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (76 mg, 1 Eq, 90 μmol), DIEA (76 mg, 0.10 mL, 6.5 Eq, 0.59 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (132 mg, 2.9 Eq, 263 μmol), and DMF (1.0 mL). The reaction mixture was stirred at 25° C. for 2 hours. The crude product was purified by Prep-HPLC using the following conditions: Column: SunFire prep OBD 19*150 mm 5 μm; Mobile Phase A: water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,18-dioxo-5,8,11, 14-tetraoxa-2, 17-diazanonadecan-19-yl)-1,4,7,10-tetraazacyclododecane-1,4,7-triyl)triacetic acid--formic acid (1/1) (59.2 mg, 46.5 μmol, 51%) as an off-white solid. Calc'd for C₅₈H₇₈C₁₂N₁₀O₁₈:1272.48, found [M+H−FA]⁺: 1227.6.

### Example 9:2,2′,2″-(10-(2-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl) piperidin-1-yl)-2-oxoethyl)-1,4,7,10-tetraazacyclododecane-1,4,7-triyl)triacetic acid (Compound 9)

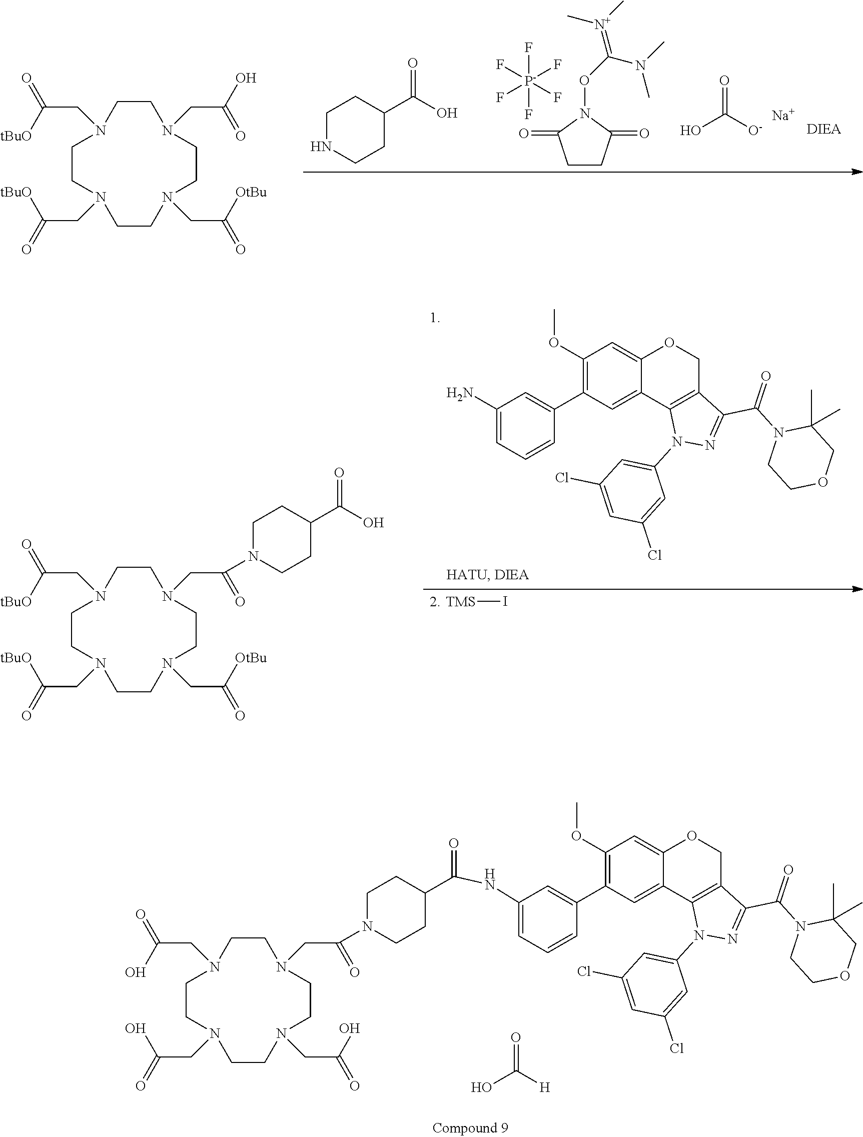

Step 1: Into a 40-mL vial, was placed a mixture of 2-(4,7,10-tris(2-(tert-butoxy)-2-oxoethyl)-1,4,7,10-tetraazacyclododecan-1-yl)acetic acid (1.2 g, 1.2 Eq, 2.1 mmol), 2-(2,5-dioxopyrrolidin-1-yl)-1,1,3,3-tetramethylisouronium hexafluorophosphate (V) (0.75 g, 1.2 Eq, 2.1 mmol), N-ethyl-N-isopropylpropan-2-amine (0.68 g, 3.0 Eq, 5.3 mmol) and THF (10 mL). The reaction mixture was stirred at 25° C. for 1 hour, then piperidine-4-carboxylic acid (0.45 g, 2.0 Eq, 3.5 mmol), sodium hydrogen carbonate (0.32 g, 2.2 Eq, 3.8 mmol), H₂O(2 mL) and THF (2 mL) were added. The resulting mixture was stirred at 25° C. for an additional 16 hours. The mixed solution was purified by Prep-HPLC using the following conditions: Column: SunFire prep OBD 19*150 mm 5 μm; Mobile Phase A: Water (0.05% NH₃H₂O); Mobile Phase B: ACN; Gradient: 25% B to 65% B in 8 min; Flow rate: 70 mL/min; Wave Length: 220 nm, using ELSD as the monitor method. The collected fractions were dried by lyophilization to provide 1-(2-(4,7,10-tris(2-(tert-butoxy)-2-oxoethyl)-1,4,7,10-tetraazacyclododecan-1-yl)acetyl) piperidine-4-carboxylic acid (525 mg, 768 μmol, 44%) as a white solid. Calc'd for C₃₄H₆₁N₅O₉:683.45, found [M+H]⁺: 684.5.

Step 2: Into an 8-mL vial, was placed a mixture of 1-(2-(4,7,10-tris(2-(tert-butoxy)-2-oxoethyl)-1,4,7,10-tetraazacyclododecan-1-yl)acetyl) piperidine-4-carboxylic acid (354 mg, 3.00 Eq, 518 μmol), HATU (210 mg, 3.20 Eq, 552 μmol), DIEA (134 mg, 181 μL, 6.01 Eq, 1.04 mmol) and DMF (1.0 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1 Eq, 173 μmol)_{w} as added and the reaction mixture was stirred at 25° C. for an additional 2 hours. The mixture was directly purified by MPLC with the following conditions: Column, WelFlash™, C18 120 g, Spherical 20-40 μm; Mobile phase, water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tri-tert-butyl 2,2′,2″-(10-(2-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl) piperidin-1-yl)-2-oxoethyl)-1,4,7,10-tetraazacyclododecane-1,4,7-triyl)triacetate (125 mg, 100 μmol, 58.2%) as a yellow oil. Calc'd for C₆₄H₈₇C₁₂N₉O₁₂:1243.58, found [M+H]⁺: 1244.7.

Step 3: Into an 8-mL vial, was placed a mixture of tri-tert-butyl 2,2′,2″-(10-(2-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl) piperidin-1-yl)-2-oxoethyl)-1,4,7,10-tetraazacyclododecane-1,4,7-triyl)triacetate (118 mg, 1 Eq, 94.8 μmol), iodotrimethylsilane solution (114 mg, 80.6 μL, 6.01 Eq, 570 μmol) and DCM (1.0 mL). The reaction mixture was stirred at 25° C. for 2 hours. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl) piperidin-1-yl)-2-oxoethyl)-1,4,7,10-tetraazacyclododecane-1,4,7-triyl)triacetic acid--formic acid (1/1) (19.4 mg, 17.3 μmol, 18.2%) as an off-white solid. Calc'd for C₅₃H₆₅C₁₂N₉O₁₄:1121.40, found [M+H−FA]⁺: 1076.5.

### Example 10:2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,6,13-trioxo-9-oxa-2,5,12-triazatetradecan-14-yl)-1,4,7,10-tetraazacyclododecane-1,4,7-triyl)triacetic acid (Compound 10)

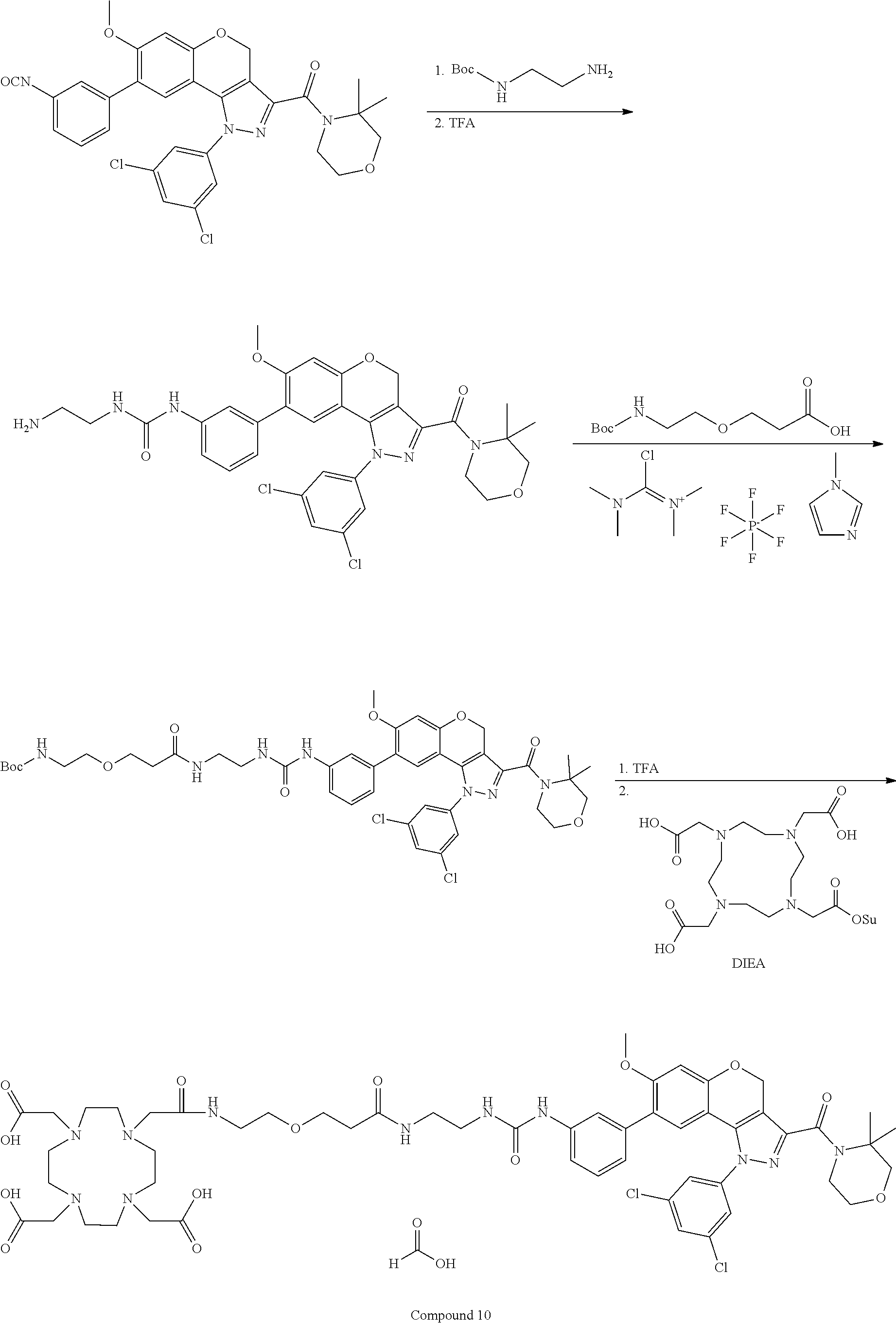

Step 1: Into a 40-mL vial, was placed a mixture of (1-(3,5-dichlorophenyl)-8-(3-isocyanatophenyl)-7-methoxy-1,4-dihydrochromeno[4,3-c]pyrazol-3-yl) (3,3-dimethylmorpholino) methanone (790 mg, 1 Eq, 1.30 mmol) and THF (8 mL), to which was added tert-butyl(2-aminoethyl) carbamate (420 mg, 2.01 eq, 2.62 mmol). The reaction mixture was stirred at 25° C. for 1 hour. The mixture was concentrated under reduced pressure. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide tert-butyl(2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl) carbamate (660 mg, 862 μmol, 66.1%) as a white solid. Calc'd for C₃₈H₄₂C₁₂N₆O₇:764.25, found [M+H−FA]⁺: 765.2, 767.2.

Step 2: Into a 40-mL round bottom flask, was placed a mixture of tert-butyl(2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl) carbamate (660 mg, 1 Eq, 862 μmol), DCM (8 mL), and TFA (0.4 mL). The reaction mixture was stirred at 25° C. for 1 hour. The collected fractions were concentrated under reduced pressure to provide 1-(2-aminoethyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (600 mg, 901 μmol, 105%) as a yellow oil. Calc'd for C₃₃H₄₄C₁₂N₆O₅:664.20, found [M+H]⁺: 665.2, 667.2.

Step 3: Into an 8-mL vial, was placed a mixture of 1-(2-aminoethyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (100 mg, 1 Eq, 150 μmol), 3-(2-((tert-butoxycarbonyl)amino) ethoxy) propanoic acid (85 mg, 2.4 Eq, 0.36 mmol), chloro-N,N,N′,N′-tetramethylformamidinium hexafluorophosphate (105 mg, 2.49 Eq, 374 μmol), 1-Methylimidazole (N—) (74 mg, 71 μL, 6.0 Eq, 0.90 mmol) and DMF (1 mL). The reaction mixture was stirred at 25° C. for 1 hour. The mixture was concentrated under reduced pressure. The crude product was purified by Prep-HPLC under the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide tert-butyl(2-(3-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3-oxopropoxy)ethyl) carbamate (63 mg, 72 μmol, 48%) as a white solid. Calc'd for C₄₃H₅₁C₁₂N—O9:879.31, found [M+H]⁺: 880.3, 882.3.

Step 4: Into an 8-mL vial, was placed a mixture of tert-butyl(2-(3-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3-oxopropoxy)ethyl) carbamate (65 mg, 1 Eq, 74 μmol) and DCM (2 mL), to which was added TFA (0.1 mL). The reaction mixture was stirred at 25° C. for 1 hour. The mixture was concentrated under reduced pressure. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 3-(2-aminoethoxy)-N-(2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl) propanamide (52 mg, 67 μmol, 90%) as a white solid. Calc'd for C₃₈H₄₃C₁₂N₇O₇:779.26, found [M+H]⁺: 780.3, 782.3.

Step 5: Into an 8-mL vial, was placed a mixture of 3-(2-aminoethoxy)-N-(2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl) propanamide (52 mg, 1 Eq, 67 μmol) and DIEA (75 mg, 0.10 mL, 8.7 Eq, 0.58 mmol), to which was added 2,2′,2″-(10-(2-((2,5-dioxopyrrolidin-1-yl)oxy)-2-oxoethyl)-1,4,7,10-tetraazacyclododecane-1,4,7-triyl)triacetic acid (75 mg, 2.2 Eq, 0.15 mmol). The reaction mixture was stirred at 25° C. for 3 hours. The mixture was concentrated under reduced pressure. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,6,13-trioxo-9-oxa-2,5,12-triazatetradecan-14-yl)-1,4,7,10-tetraazacyclododecane-1,4,7-triyl)triacetic acid--formic acid (1/1) (14.3 mg, 11.8 μmol, 18%) as a white solid. Calc'd for C₅₅H₇₁C₁₂N₁₁O₁₆:1211.45, found [M+H−FA]⁺: 1166.5, 1168.5.

### Example 11:2,2′,2″-(10-(2-(4-(4-((3-(1-(3,5-dichlorophenyl)-3-(3,3-dimethylmorpholine-4-carbonyl)-7-methoxy-1,4-dihydrochromeno|4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid (Compound 11)

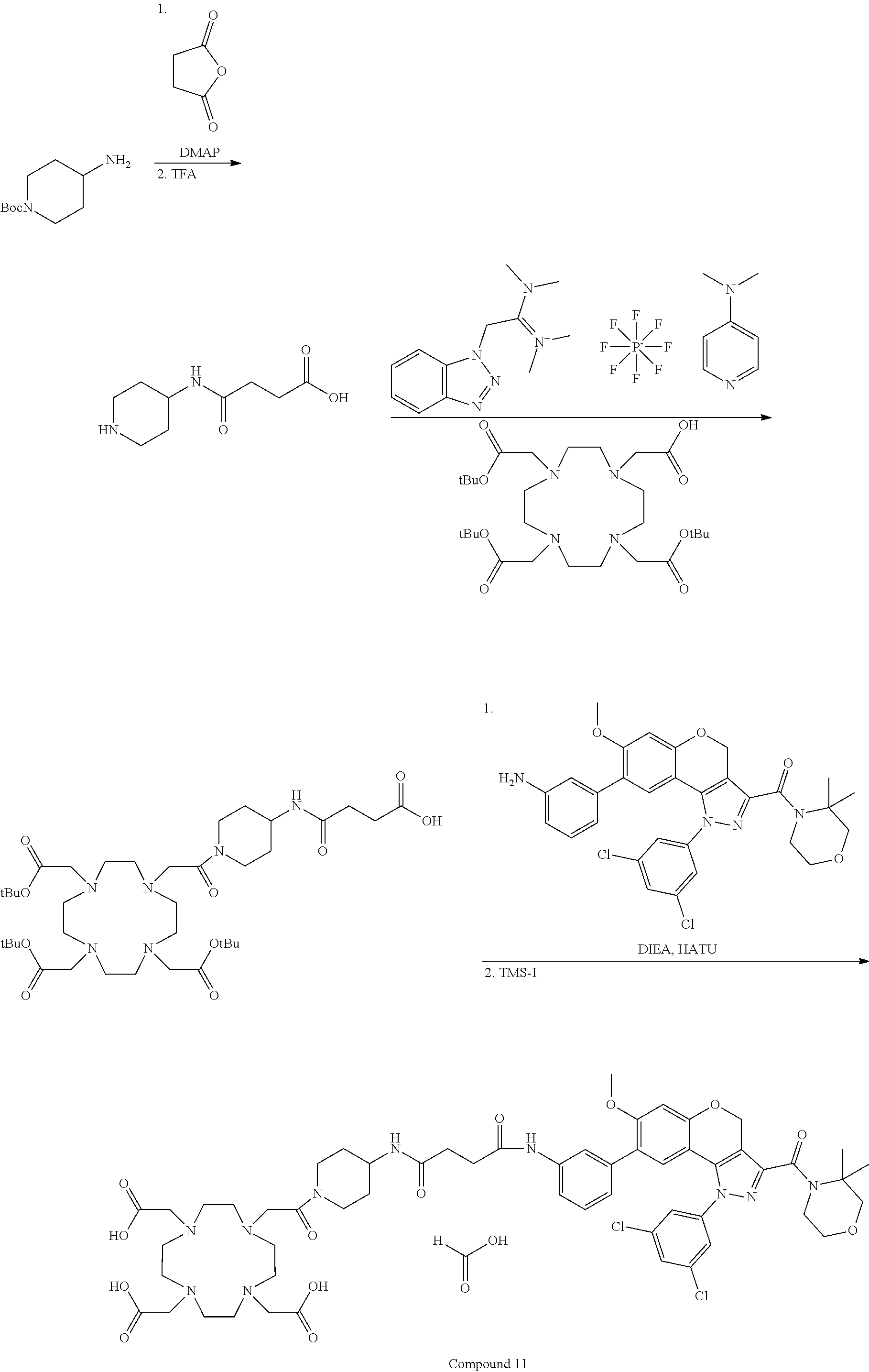

Step 1: Into a 250-mL vial, was placed a mixture of tert-butyl 4-aminopiperidine-1-carboxylate (10 g, 1 Eq, 50 mmol), dihydrofuran-2,5-dione (1.2 g, 0.24 Eq, 12 mmol), DMAP (0.30 g, 0.049 Eq, 2.5 mmol) and DCM (40 mL). The reaction mixture was stirred at 25° C. for 2 hours, then an aqueous solution of sodium bicarbonate (200 mL)_{w} as added. The water layer was extracted with EtOAc (200 ml×3), The pH of the water phase was adjusted to 3 by addition of a 2N HCl solution, then the water layer was extracted with EtOAc (200 ml×3). The combined organic layers were washed with brine (200 mL), dried over anhydrous Na₂SO₄ and concentrated under reduced pressure to provide 4-((1-(tert-butoxycarbonyl) piperidin-4-yl)amino)-4-oxobutanoic acid (8.0 g, 27 mmol, 53%) as a white solid. Calc'd for C₁₄H₂₄N₂O₅:300.16, found [M+H]⁺: 301.3.

Step 2: Into a 40-mL vial, was placed a mixture of 4-((1-(tert-butoxycarbonyl) piperidin-4-yl)amino)-4-oxobutanoic acid (1.0 g, 1 Eq, 3.3 mmol) and 4M HCl in dioxane (20 mL). The reaction mixture was stirred at 25° C. for 1 hour. NaHCO₃ aqueous solution was added to the mixed solution to adjust the pH to 9, and then concentrated under reduced pressure to provide 4-oxo-4-(piperidin-4-ylamino) butanoic acid (0.85 g, 3.4 mmol, 100%, 79% Purity) as a white solid. Calc'd for C₉H₁₆N₂O₃:300.11, found [M+H]⁺: 201.1.

Step 3: Into a 40-mL vial, was placed a mixture of 4-oxo-4-(piperidin-4-ylamino) butanoic acid (813 mg, 3.00 Eq, 4.06 mmol), 2-(4,7,10-tris(2-(tert-butoxy)-2-oxoethyl)-1,4,7,10-tetraazacyclododecan-1-yl)acetic acid (0.775 g, 1 Eq, 1.35 mmol), HBTU (616 mg, 1.20 Eq, 1.62 mmol), DMAP (10 mg, 0.060 Eq, 82 μmol) and DMF (10 mL). The reaction mixture was stirred at 25° C. for 4 hours. The mixed solution was purified by Prep-HPLC using the following conditions: Column: SunFire prep OBD 19*150 mm 5 μm; Mobile Phase A: Water (0.05% NH₃H₂O); Mobile Phase B: ACN; Gradient: 25% B to 65% B in 8 min; Flow rate: 70 mL/min; Wave Length: 220 nm, using ELSD as the monitoring method. The collected fractions were dried by lyophilization to provide 4-oxo-4-((1-(2-(4,7,10-tris(2-(tert-butoxy)-2-oxoethyl)-1,4,7,10-tetraazacyclododecan-1-yl)acetyl) piperidin-4-yl)amino) butanoic acid (490 mg, 649 μmol, 48.0%) as a white solid. Calc'd for C₃₇H₆₆N₆O₁₀:754.48, found [M+H]⁺: 755.5.

Step 4: Into an 8-mL vial, was placed a mixture of 4-oxo-4-((1-(2-(4,7,10-tris(2-(tert-butoxy)-2-oxoethyl)-1,4,7,10-tetraazacyclododecan-1-yl)acetyl) piperidin-4-yl)amino) butanoic acid (100 mg, 1 Eq, 132 μmol) and DMF (1 mL), to which was added HATU (80 mg, 1.6 Eq, 0.21 mmol) and DIEA (55 mg, 74 μL, 3.2 Eq, 0.43 mmol). The reaction mixture was stirred at 23° C. for 30 mins, then (8-(3-aminophenyl)-1-(3,5- dichlorophenyl)-7-methoxy-1,4-dihydrochromeno[4,3-c]pyrazol-3-yl) (3,3-dimethylmorpholino) methanone (70 mg, 0.91 Eq, 0.12 mmol)_{w} as added. The reaction mixture was stirred at 23° C. for 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 2 min, 30% ACN up to 98% in 10 min, 98% ACN to 98% in 1 min); Total flow rate, 70 mL/min; Detector, UV 220 nm. The collected fractions were concentrated to afford tri-tert-butyl 2,2′,2″-(10-(2-(4-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetate (150 mg, 114 μmol, 86.0%) as a yellow solid. Calc'd for C₆₇H₉₂C₁₂N₁₀O₁₃:1314.62, found [M+H]⁺: 1315.5, 1317.5.

Step 5: Into an 8-mL vial, was placed a mixture of tri-tert-butyl 2,2′,2″-(10-(2-(4-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetate (150 mg, 1 Eq, 114 μmol), TMSI (140 mg, 95.2 μL, 6.14 Eq, 700 μmol), and DCM (1.5 mL). The reaction mixture was stirred at 20° C. for 1 hour. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 10 min; Flow rate: 20 mL/min; Wave Length: 220 nm. The collected fractions were dried by lyophilization to provide 2,2′,2″-(10-(2-(4-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid--formic acid (1/1) (49.2 mg, 41.2 μmol, 36.2%) as a white solid. Calc'd for C₅₆H₇₀Cl₂N₁₀₀Is: 1192.44, found [M+H−FA]⁺: 1147.5, 1149.5.

### Example 12: (R)-2,2′,2″-(10-(2-(4-(2-amino-4-((3-(1-(3,5-dichlorophenyl)-3-(3,3-dimethylmorpholine-4-carbonyl)-7-methoxy-1,4-dihydrochromeno|4,3-clpyrazol-8-yl)phenyl)amino)-4-oxobutanamido) piperidin-1-yl)-2-oxoethyl)-1,4,7,10-tetraazacyclododecane-1,4,7-triyl)triacetic acid (Compound 12)

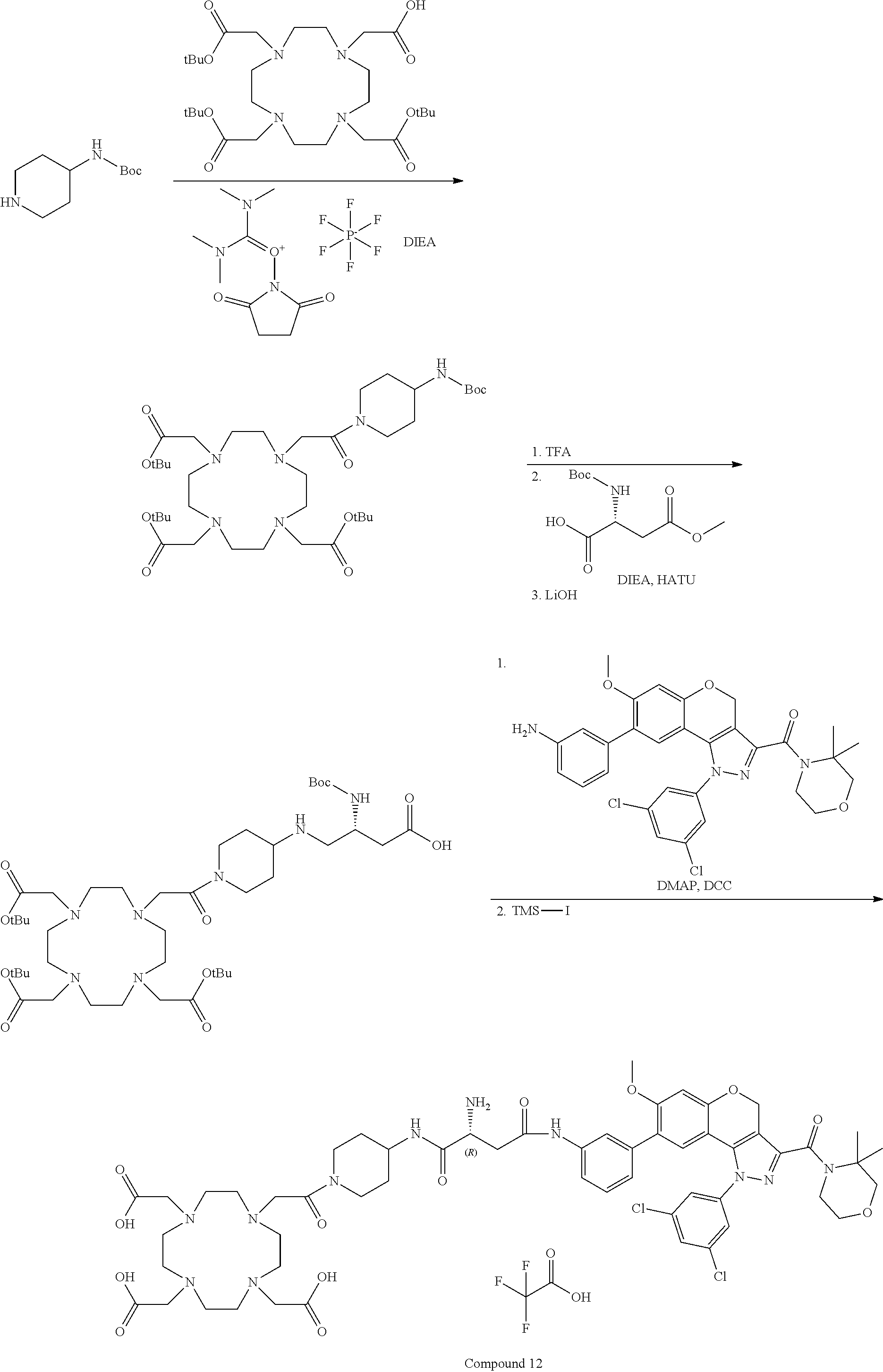

Step 1: Into a 250-mL round-bottom flask, was placed 2-(4,7,10-tris(2-(tert-butoxy)-2-oxoethyl)-1,4,7,10-tetraazacyclododecan-1-yl)acetic acid (2.0 g, 1 Eq, 3.5 mmol), DMF (20 mL), DIEA (1.8 g, 2.4 mL, 4.0 Eq, 14 mmol), and N,N,N,N-tetramethyl-o-(n-succinimidyl)uronium hexafluorophosphate (1.7 g, 1.4 Eq, 4.7 mmol). The reaction mixture was stirred at 25° C. for 5 minutes, then tert-butyl piperidin-4-ylcarbamate (1.1 g, 1.6 Eq, 5.5 mmol)_{w} as added and the reaction mixture was stirred at 25° C. for an additional 1 hour. The reaction mixture was purified by MPLC using the following conditions: Column, C18 120 g; mobile phase, Water (0.1% FA) and ACN (30% ACN up to 98% in 6 min, 98% ACN to 98% in 3 min); Total flow rate, 70 mL/min; Detector, UV 220 nm. The collected fractions were concentrated under reduced pressure and dried to provide tri-tert-butyl 2,2′,2″-(10-(2-(4-((tert-butoxycarbonyl)amino) piperidin-1-yl)-2-oxoethyl)-1,4,7,10-tetraazacyclododecane-1,4,7-triyl)triacetate (2.0 g, 2.6 mmol, 76%) as a yellow solid. Calc'd for C₃₈H₇₀N₆O₉:754.52, found [M+H]⁺: 755.5.

Step 2: Into a 100-mL round-bottom flask, was placed tri-tert-butyl 2,2′,2″-(10-(2-(4-((tert-butoxycarbonyl)amino) piperidin-1-yl)-2-oxoethyl)-1,4,7,10-tetraazacyclododecane-1,4,7-triyl)triacetate (1.5 g, 1 Eq, 2.0 mmol), DCM (15 mL) and TFA (3 mL). The mixture was stirred for 1 hour at 25° C. The reaction mixture was concentrated under reduced pressure to provide tri-tert-butyl 2,2′,2″-(10-(2-(4-aminopiperidin-1-yl)-2-oxoethyl)-1,4,7,10-tetraazacyclododecane-1,4,7-triyl)triacetate 2,2,2-trifluoroacetate (1.5 g, 1.6 mmol, 79%, 80% Purity) as a yellow oil. Calc'd for C₃₅H₆₃F3N₆O₉:768.46, found [M+H−TFA]⁺: 655.5. Step 3: Into a 100-mL round-bottom flask, was placed (R)-2-((tert-butoxycarbonyl)amino)-4-methoxy-4-oxobutanoic acid (0.53 g, 1.4 Eq, 2.1 mmol), DMF (15 mL) and DIEA (1.0 g, 1.3 mL, 5.0 Eq, 7.7 mmol), to which was added HATU (0.81 g, 1.4 Eq, 2.1 mmol). The mixture was stirred for 20 mins at 25° C., then tri-tert-butyl 2,2′,2″-(10-(2-(4-aminopiperidin-1-yl)-2-oxoethyl)-1,4,7,10-tetraazacyclododecane-1,4,7-triyl)triacetate 2,2,2-trifluoroacetate (1.5 g, 80% wt, 1 Eq, 1.6 mmol)_{w} as added. The reaction mixture was stirred at 25° C. for an additional 1 hour. The reaction mixture was purified by MPLC using the following conditions: Column, C18 120 g; mobile phase, Water (0.1% FA) and ACN (30% ACN up to 98% in 6 min, 98% ACN to 98% in 3 min); Total flow rate, 70 mL/min; Detector, UV 220 nm. The collected fractions were concentrated under reduced pressure and dried to provide tri-tert-butyl 2,2′,2″-(10-(2-(4-(2-((tert-butoxycarbonyl)amino)-4-methoxy-4-oxobutanamido) piperidin-1-yl)-2-oxoethyl)-1,4, 7, 10-tetraazacyclododecane-1,4,7-triyl) (R)-triacetate (880 mg, 995 μmol, 64%) as a yellow solid. Calc'd for C₄₃H₇₇N₇O₁₂:883.56, found [M+H]: 884.6.

Step 4: Into a 40-mL vial, was placed a mixture of tri-tert-butyl 2,2′,2″-(10-(2-(4-(2-((tert-butoxycarbonyl)amino)-4-methoxy-4-oxobutanamido) piperidin-1-yl)-2-oxoethyl)-1,4,7,10-tetraazacyclododecane-1,4,7-triyl) (R)-triacetate (880 mg, 1 Eq, 995 μmol), MeOH (10 mL), LiOH (50 mg, 2.1 Eq, 2.1 mmol), and H₂O(2 mL). The reaction mixture was stirred at 20° C. for 2 hours. The reaction mixture was concentrated under reduced pressure to remove most of the MeOH, the residue was diluted with water (30 mL), and the pH value was adjusted to 6.0 by addition of a saturated NaHSO₄ solution. The solution was extracted with DCM (50 mL×3), dried over anhydrous Na₂SO₄, and concentrated under reduced pressure to afford (R)-3-((tert-butoxycarbonyl)amino)-4-oxo-4-((1-(2-(4,7,10-tris(2-(tert-butoxy)-2-oxoethyl)-1,4,7,10-tetraazacyclododecan-1-yl)acetyl) piperidin-4-yl)amino) butanoic acid (700 mg, 805 μmol, 80.8%) as a light yellow solid, which was used directly in the next step without any purification. Calc'd for C₄₂H₇₅N₇O₁₂:869.55, found [M+H]⁺: 870.5.

Step 5: Into an 8-mL vial, was placed a mixture of (R)-3-((tert-butoxycarbonyl)amino)-4-oxo-4-((1-(2-(4,7,10-tris(2-(tert-butoxy)-2-oxoethyl)-1,4,7,10-tetraazacyclododecan-1-yl)acetyl) piperidin-4-yl)amino) butanoic acid (100 mg, 1 Eq, 115 μmol) in DCM (1 mL), to which was added (8-(3-aminophenyl)-1-(3,5-dichlorophenyl)-7-methoxy-1,4-dihydrochromeno[4,3-c]pyrazol-3-yl) (3,3-dimethylmorpholino) methanone (67 mg, 1.0 Eq, 0.12 mmol) and DMAP (7 mg, 0.5 Eq, 0.06 mmol). The reaction was stirred at 0° C., then DCC (36 mg, 1.5 Eq, 0.17 mmol)_{w} as added. The resulting mixture was stirred at 0° C. to 25° C. for 2 hours. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide tri-tert-butyl 2,2′,2″-(10-(2-(4-(2-((tert-butoxycarbonyl)amino)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl) (R)-triacetate (60 mg, 42 μmol, 36%) as a light yellow oil. Calc'd for C₇₂H₁₀₁C₁₂N₁₁O₁₅:1429.69, found [M+H]: 1430.9, 1432.9.

Step 6: Into an 8-mL vial, was placed a mixture of tri-tert-butyl 2,2′,2″-(10-(2-(4-(2-((tert-butoxycarbonyl)amino)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl) (R)-triacetate (60 mg, 1 Eq, 42 μmol) in DCM (1 mL). The reaction was cooled 0° C. with stirring, then iodotrimethylsilane (50 mg, 6.0 Eq, 0.25 mmol)_{w} as added. The resulting mixture was stirred at 0° C. to 25° C. for 1 hour. To the reaction mixture was added a IN aqueous solution of NaOH (0.25 mL) and a saturated solution of NaHCO₃ at 0° C. The resulting mixture was concentrated and purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(2-(4-(2-amino-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid--2,2,2-trifluoroacetic acid (1/1) (6.8 mg, 5.1 μmol, 12%, 96% Purity) as a white solid. Calc'd for C₅₇H₇₀Cl₂F₃N₁₁O₁₅:1275.44, found [M+H−TFA]⁺: 1162.4, 1164.4.

### Example 13: (Compound 13)

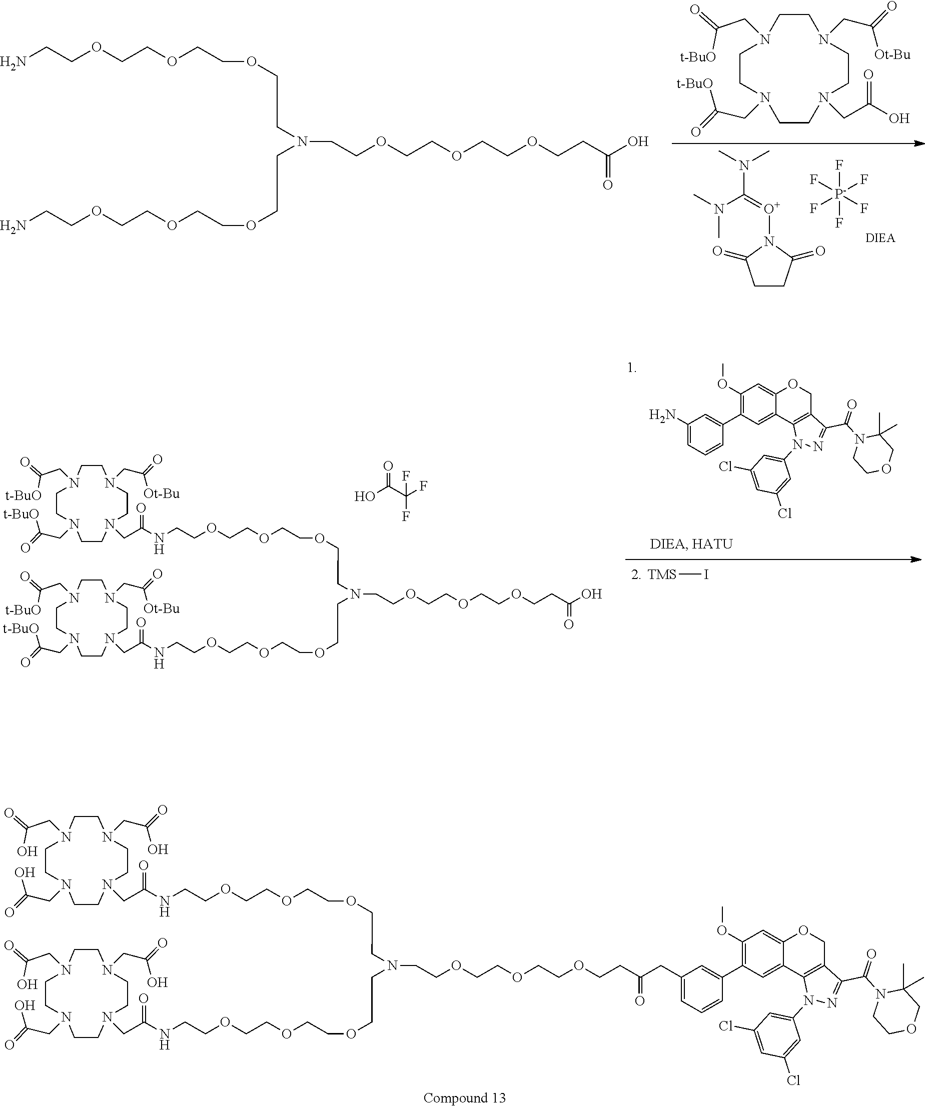

Step 1: Into a 40-mL vial, was placed a mixture of 2-(4,7,10-tris(2-(tert-butoxy)-2-oxoethyl)-1,4,7,10-tetraazacyclododecan-1-yl)acetic acid (1.15 g, 2.30 Eq, 2.01 mmol), N-ethyl-N-isopropylpropan-2-amine (565 mg, 5.00 Eq, 4.37 mmol), 2-(2,5-dioxopyrrolidin-1-yl)-1,1,3,3-tetramethyluronium hexafluorophosphate (V) (785 mg, 2.50 Eq, 2.19 mmol) and DMF (5 mL). The mixture was stirred at 25° C. for 1 hour, then 1-amino-12-(2-(2-(2-(2-aminoethoxy)ethoxy)ethoxy)ethyl)-3,6,9, 15,18,21-hexaoxa-12-azatetracosan-24-oic acid (500 mg, 1 Eq, 875 μmol)_{w} as added. The reaction mixture was stirred at 25° C. for 1 hour. The mixture was concentrated under reduced pressure and purified by Prep-HPLC using the following conditions: Column, SunFire Prep C18 OBD Column, 19*150 mm, 5 um; mobile phase, Water (0.1% TFA) and ACN (20% ACN up to 65% in 7 min); Total flow rate, 20 mL/min; Detector, UV 220 nm. Pure fractions were dried by lyophilization to provide 2,2,2-trifluoroacetic acid--2-oxo-15-(2-oxo-1-(4,7,10-tris(2-(tert-butoxy)-2-oxoethyl)-1,4,7,10-tetraazacyclododecan-1-yl)-6,9, 12-trioxa-3-azatetradecan-14-yl)-1-(4,7,10-tris(2-(tert-butoxy)-2-oxoethyl)-1,4,7,10-tetraazacyclododecan-1-yl)-6,9, 12, 18,21,24-hexaoxa-3, 15-diazaheptacosan-27-oic acid (1/1) (750 mg, 0.41 mmol, 47%, 98% Purity) as a colorless oil. Calc'd for C₈₃H₁₅₄F₃N₁₁O₂₇:1794.10, found [M+H−TFA]⁺: 1681.2.

Step 2: Into a 40-mL vial, was placed a mixture of 2-oxo-15-(2-oxo-1-(4,7,10-tris(2-(tert-butoxy)-2-oxoethyl)-1,4,7,10-tetraazacyclododecan-1-yl)-6,9, 12-trioxa-3-azatetradecan-14-yl)-1-(4,7,10-tris(2-(tert-butoxy)-2-oxoethyl)-1,4,7,10-tetraazacyclododecan-1-yl)-6,9,12, 18,21,24-hexaoxa-3, 15-diazaheptacosan-27-oic acid (300 mg, 3 Eq, 178 μmol) in DMF (3 mL), then HATU (82 mg, 3.6 Eq, 0.22 mmol) and DIEA (46 mg, 62 μL, 6.0 Eq, 0.36 mmol) were added. The mixture was stirred at 25° C. for 10 mins. To the above mixture was added (8-(3-aminophenyl)-1-(3,5-dichlorophenyl)-7-methoxy-1,4-dihydrochromeno[4,3-c]pyrazol-3-yl) (3,3-dimethylmorpholino) methanone (35 mg, 1.0 Eq, 60 μmol). The resulting mixture was stirred at 25° C. for 2 hours. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide the product (140 mg, 56 μmol, 94%, 90% Purity) as a light yellow oil. Calc'd for C₁₁₁H₁₇₉C₁₂N₁₅O₂₈:2240.24, found [M/2+H]⁺: 1121.9, 1123.9.

Step 3: Into an 8-mL vial, was placed the product from Step 2 (70 mg, 1 Eq, 31 μmol) and DCM (1 mL), to which was added iodotrimethylsilane (120 mg, 19 Eq, 600 μmol). The reaction mixture was stirred at 0° C. to 25° C. for 2 hours. The mixture was concentrated under reduced pressure. The crude product was purified by Prep-HPLC using the following conditions: Column: SunFire prep OBD 19*150 mm 5 μm; Mobile Phase A: Water (0.05% TFA); Mobile

Phase B: ACN; Gradient: 25% B to 65% B in 8 min; Flow rate: 20 mL/min; Wave Length: 220 nm. The collected fractions were dried by lyophilization to provide the title compound (14.6 mg, 5.83 μmol, 19%, 94.1% Purity) as an off-white solid. Calc'd for C₈₉H₁₃₂C₁₂F3N₁₅O₃₀:2017.86, found [M+H−TFA]⁺: 1904.8, 1906.8.

### Example 14:2,2′,2″-(10-(2-(((3R,5R)-7-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,5-dihydroxy-7-oxoheptyl)amino)-2-oxoethyl)-1,4,7,10-tetraazacyclododecane-1,4,7-triyl)triacetic acid (Compound 14)

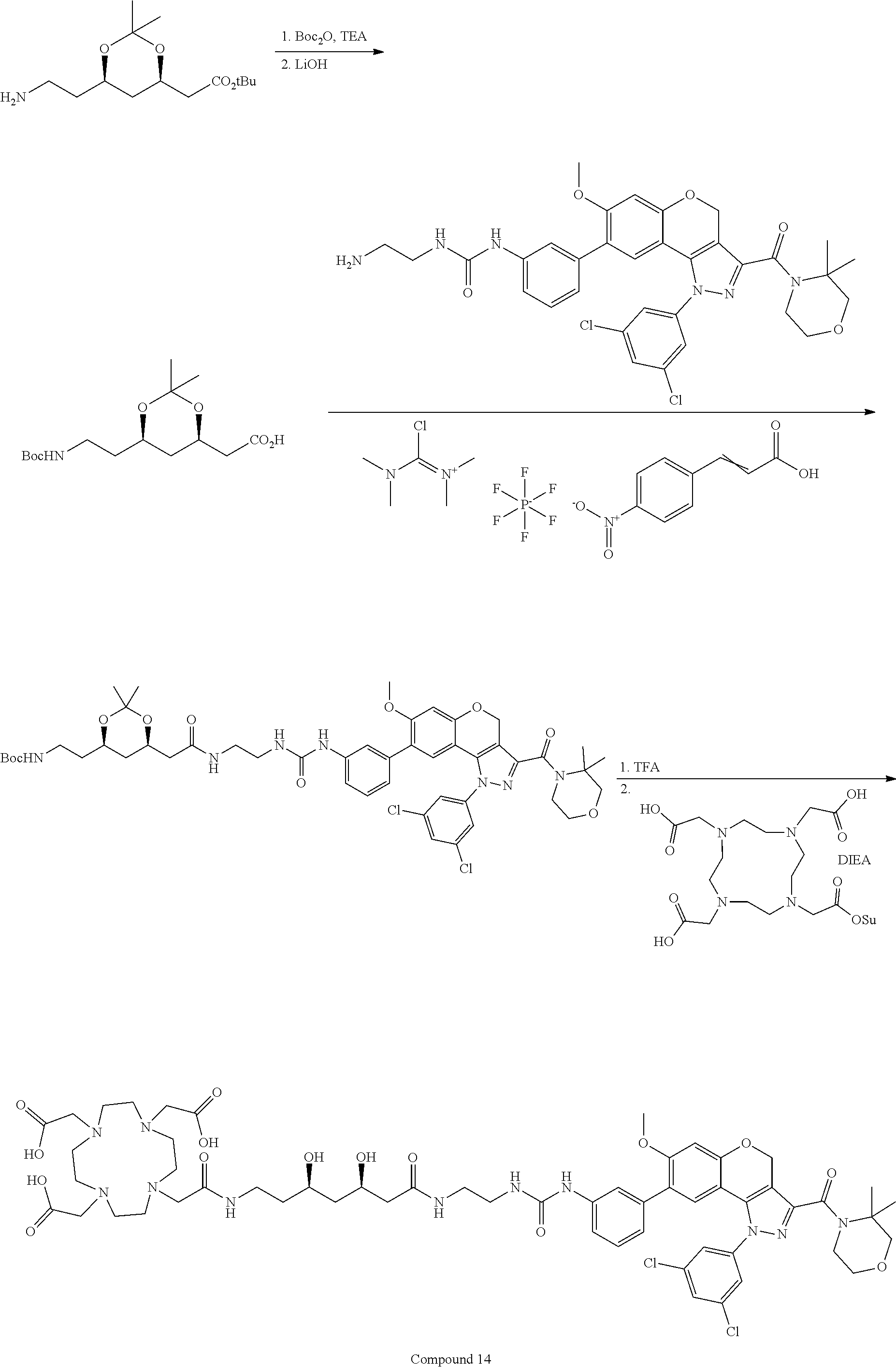

Step 1: Into a 100-mL round bottom flask, was placed a mixture of tert-butyl 2-((4R,6R)-6-(2-aminoethyl)-2,2-dimethyl-1,3-dioxan-4-yl)acetate (2 g, 1 Eq, 7 mmol), TEA (2 g, 3 mL, 3 Eq, 0.02 mol), Boc₂O(2 g, 3 mL, 1.5 Eq, 0.01 mol) and DCM (20 mL). The reaction mixture was stirred at 25° C. for 1 hour. The mixture was purified by MPLC using the following conditions: Silica gel column 40 g, PE/EtOAc system, the ratio of EtOAc from 0% to 85% in 15 min, Flow rate: 40 mL/min; Wave Length: 254 nm. The collected fractions were concentrated under reduced pressure to provide tert-butyl 2-((4R,6R)-6-(2-((tert-butoxycarbonyl)amino)ethyl)-2,2-dimethyl-1,3-dioxan-4-yl)acetate (2.34 g, 6.27 mmol, 90%) as a yellow oil. Calc'd for C₁₉H₃₅NO₆:373.25, found [M+H]⁺: 374.2.

Step 2: Into a 40-mL round bottom flask, was placed a mixture of tert-butyl 2-((4R,6R)-6-(2-((tert-butoxycarbonyl)amino)ethyl)-2,2-dimethyl-1,3-dioxan-4-yl)acetate (2.34 g, 1 Eq, 6.27 mmol), LiOH (3.00 g, 20 Eq, 125 mmol), H₂O(2 g, 2 mL, 2e+1 Eq, 0.1 mol) and MeOH (6 mL). The reaction mixture was stirred at 25° C. for 16 hours. The mixture was diluted with water (100 mL), extracted with EtOAc (50 mL×3), then the combined organic layers were washed with water (50 mL×2), brine (50 mL), dried over anhydrous Na₂SO₄ and concentrated under reduced pressure to provide 2-((4R,6R)-6-(2-((tert-butoxycarbonyl)amino)ethyl)-2,2-dimethyl-1,3-dioxan-4-yl)acetic acid (1.37 g, 4.32 mmol, 68.9%) as a yellow oil. Calc'd for C₁₅H₂₇NO₆:317.18, found [M+H]⁺: 318.2.

Step 3: Into an 8-mL vial, was placed a mixture of 2-((4R,6R)-6-(2-((tert-butoxycarbonyl)amino)ethyl)-2,2-dimethyl-1,3-dioxan-4-yl)acetic acid (175 mg, 2.45 Eq, 551 μmol), chloro-N,N,N′,N′-tetramethylformamidinium hexafluorophosphate (190 mg, 3.00 Eq, 677 μmol), 1-methylimidazole (N—) (92 mg, 89 μL, 5.0 Eq, 1.1 mmol) and DMF (1.5 mL). The reaction mixture was stirred at 20° C. for 10 minutes, then 1-(2-aminoethyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (See Example 10, Step 2, 150 mg, 1 Eq, 225 μmol)_{w} as added and the reaction mixture was stirred at 25° C. for an additional 1 hour. The crude mixture was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide tert-butyl(2-((4R,6R)-6-(2-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-2-oxoethyl)-2,2-dimethyl-1,3-dioxan-4-yl)ethyl) carbamate (60 mg, 62 μmol, 28%) as a white solid. Calc'd for C₄₈H₅₉C₁₂N₇O₁₀:963.37, found [M+H]⁺: 964.4, 966.4.

Step 4: Into a 40-mL round bottom flask, was placed a mixture of tert-butyl(2-((4R,6R)-6-(2-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-2-oxoethyl)-2,2-dimethyl-1,3-dioxan-4-yl)ethyl) carbamate (60 mg, 1 Eq, 62 μmol), TFA (0.1 mL), and DCM (2 mL). The reaction mixture was stirred at 25° C. for 20 min. This resulted in (3R,5R)-7-amino-N-(2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)-3,5-dihydroxyheptanamide (43 mg, 52 μmol, 84%) as a yellow oil. Calc'd for C₄₀H₄₇C₁₂N—O8:823.29, found [M+H]⁺: 824.3, 826.3.

Step 5: Into an 8-mL vial, was placed a mixture of (3R,5R)-7-amino-N-(2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)-3,5-dihydroxyheptanamide (65 mg, 1 Eq, 79 μmol), N-ethyl-N-isopropylpropan-2-amine (80 mg, 7.9 Eq, 0.62 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (80 mg, 2.0 Eq, 0.16 mmol), and DMF (1 mL). The reaction mixture was stirred at 25° C. for 3 hours. The mixture was concentrated under reduced pressure. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-(((3R,5R)-7-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,5-dihydroxy-7-oxoheptyl)amino)-2-oxoethyl)-1,4,7,10-tetraazacyclododecane-1,4,7-triyl)triacetic acid (18.9 mg, 15.6 μmol, 20%) as a white solid. Calc'd for C₅₆H₇₃C₁₂N₁₁O₁₅:1209.47, found [M+H]⁺: 1210.5, 1212.5.

### Example 15:2,2′,2″-(10-(2-(4-(N-(2-(2-(2-carboxyethoxy)ethoxy)ethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid (Compound 15)

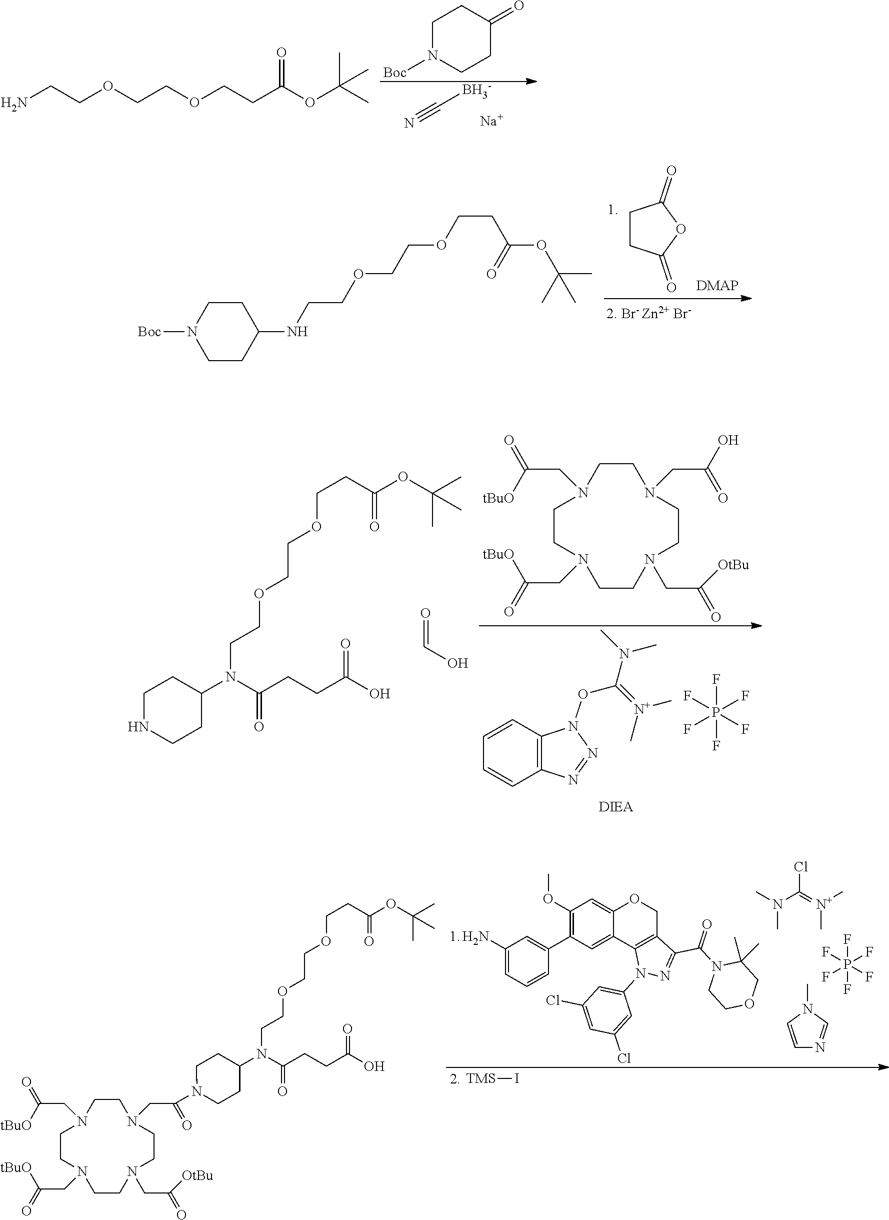

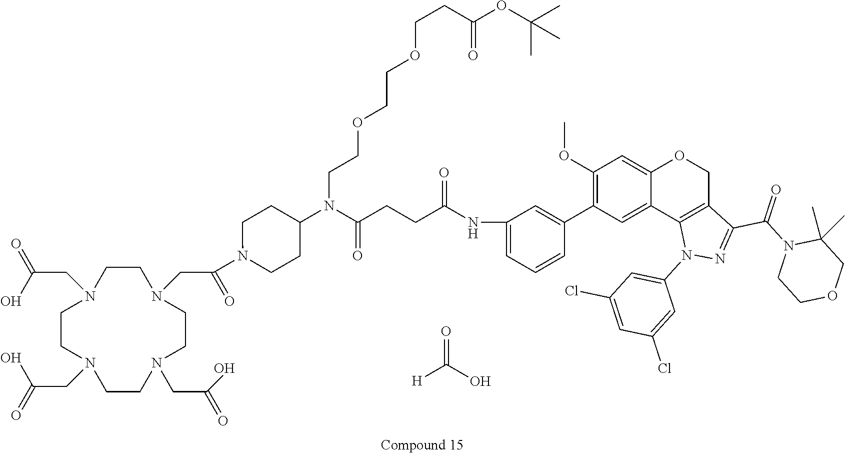 [SMILES: COc1cc2c(cc1-c1cccc(NC(=O)CCC(=O)N(CCOCCOCCC(=O)OC(C)(C)C)C3CCN(C(=O)CN4CCN(CC(=O)O)CCN(CC(=O)O)CCN(CC(=O)O)CC4)CC3)c1)-c1c(c(C(=O)N3CCOCC3(C)C)nn1-c1cc(Cl)cc(Cl)c1)CO2 ; O=CO]

Step 1: Into a 500-mL round bottom flask, was placed a mixture of tert-butyl 4-oxopiperidine-1-carboxylate (2.3 g, 1 Eq, 12 mmol), tert-butyl 3-(2-(2-aminoethoxy)ethoxy) propanoate (3.5 g, 1.3 Eq, 15 mmol) and DCE (25 mL). The reaction mixture was stirred at 20° C. for 50 minutes, then sodium cyanoborohydride (2.15 g, 2.02 mL, 3.0 Eq, 34.2 mmol)_{w} as added and the reaction mixture was stirred at 25° C. for an additional 3 days. The crude product was purified by MPLC with the following conditions: Silica gel column 120 g, PE/EtOAc system, the ratio of EtOAc from 0% to 85% in 15 min, Flow rate: 90 mL/min; Wave Length: 254 nm. The collected fractions were concentrated under reduced pressure to provide tert-butyl 4-((2-(2-(3-(tert-butoxy)-3-oxopropoxy)ethoxy)ethyl)amino) piperidine-1-carboxylate (2.2 g, 5.3 mmol, 46%) as a yellow oil. Calc'd for C₂₁H₄₀N₂O₆:416.29, found [M+H]⁺: 417.5.

Step 2: Into a 40-mL vial, was placed a mixture of tert-butyl 4-((2-(2-(3-(tert-butoxy)-3-oxopropoxy)ethoxy)ethyl)amino) piperidine-1-carboxylate (1.0 g, 1 Eq, 2.4 mmol), dihydrofuran-2,5-dione (0.5 g, 2 Eq, 5 mmol), DMAP (0.9 g, 3 Eq, 7 mmol) and CICH2CH₂Cl (15 mL). The reaction mixture was stirred at 25° C. for 24 hours. The mixture was diluted with 100 ml of water, extracted with EtOAc (150 mL×3), then the combined organic layers were washed with water (150 mL×2), brine (150 mL), dried over anhydrous Na₂SO₄ and concentrated under reduced pressure. The crude product was purified by MPLC with the following conditions: Silica gel column 80 g, PE/EtOAc system, the ratio of EtOAc from 0% to 85% in 15 min, Flow rate: 70 mL/min; Wave Length: 254 nm. The collected fractions were concentrated under reduced pressure to provide 13-(1-(tert-butoxycarbonyl) piperidin-4-yl)-2,2-dimethyl-4,14-dioxo-3,7, 10-trioxa-13-azaheptadecan-17-oic acid (920 mg, 1.78 mmol, 74%) as a yellow oil. Calc'd for C₂₅H₄₄N₂O₉:516.30, found [M+H]⁺: 517.5.

Step 3: Into a 40-mL vial, was placed a mixture of 13-(1-(tert-butoxycarbonyl) piperidin-4-yl)-2,2-dimethyl-4,14-dioxo-3,7, 10-trioxa-13-azaheptadecan-17-oic acid (1.0 g, 1 Eq, 1.9 mmol), ZnBr₂ (2 g, 5 Eq, 9 mmol) and DCM (10 mL). The reaction mixture was stirred at 25° C. for 16 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide 2,2-dimethyl-4, 14-dioxo-13-(piperidin-4-yl)-3,7,10-trioxa-13-azaheptadecan-17-oic acid--formic acid (1/1) (440 mg, 951 μmol, 49%) as a yellow oil. Calc'd for C₂₁H₃₈N₂O₉:462.57, found [M+H−FA]: 417.5.

Step 4: Into a 40-mL vial, was placed a mixture of 2-(4,7,10-tris(2-(tert-butoxy)-2-oxoethyl)-1,4,7,10-tetraazacyclododecan-1-yl)acetic acid (817 mg, 1.50 Eq, 1.43 mmol), HBTU (722 mg, 2.00 Eq, 1.90 mmol), DIEA (615 mg, 829 μL, 5.00 Eq, 4.76 mmol) and DMF (5 mL). The reaction mixture was stirred at 25° C. for 10 minutes, then 2,2-dimethyl-4, 14-dioxo-13-(piperidin-4-yl)-3,7, 10-trioxa-13-azaheptadecan-17-oic acid--formic acid (1/1) (440 mg, 1 Eq, 951 μmol), was added and the reaction mixture was stirred at 25° C. for an additional 1 hour. The crude product was purified by Prep-HPLC with the following conditions: Column: SunFire prep OBD 19*150 mm 5 μm; Mobile Phase A: Water (0.05% NH₃.H₂O); Mobile Phase B: ACN; Gradient: 25% B to 65% B in 8 min; Flow rate: 20 mL/min; Wave Length: 220 nm. The collected fractions were dried by lyophilization to provide 2,2-dimethyl-4, 14-dioxo-13-(1-(2-(4,7,10-tris(2-(tert-butoxy)-2-oxoethyl)-1,4,7,10-tetraazacyclododecan-1-yl)acetyl) piperidin-4-yl)-3,7,10-trioxa-13-azaheptadecan-17-oic acid (250 mg, 257 μmol, 27.1%) as a yellow oil. Calc'd for C₄₈H₈₆N₆O₁₄:970.60, found [M+H]: 972.2.

Step 5: Into an 8-mL vial, was placed a mixture of 2,2-dimethyl-4, 14-dioxo-13-(1-(2-(4,7,10-tris(2-(tert-butoxy)-2-oxoethyl)-1,4,7,10-tetraazacyclododecan-1-yl)acetyl) piperidin-4-yl)-3,7,10-trioxa-13-azaheptadecan-17-oic acid (120 mg, 1 Eq, 124 μmol), 1-methyl-1H-imidazole (30.4 mg, 3.00 Eq, 370 μmol), N-(chloro (dimethylamino)methylene)-N-methylmethanaminium hexafluorophosphate (V) (69.3 mg, 2.00 Eq, 247 μmol) and DCM (1.5 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (107 mg, 1.49 Eq, 185 μmol)_{w} as added and the reaction mixture was stirred at 25° C. for an additional 1 hour. The mixture was directly purified by MPLC with the following conditions: Column, WelFlash™, C18 120 g, Spherical 20-40 μm; Mobile phase, water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide tri-tert-butyl 2,2′,2″-(10-(2-(4-(N-(2-(2-(3-(tert-butoxy)-3-oxopropoxy)ethoxy)ethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetate formate (95 mg, 60 μmol, 49%) as a yellow oil. Calc'd for C₇₉H₁₁₄C₁₂N₁₀O₁₉:1576.76, found [M+H−FA]: 1533.7.

Step 6: Into an 8-mL vial, was placed a mixture of tri-tert-butyl 2,2′,2″-(10-(2-(4-(N-(2-(2-(3-(tert-butoxy)-3-oxopropoxy)ethoxy)ethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetate formate (60 mg, 1 Eq, 38 μmol), and ACN (0.5 mL), to which was added TMS-I (63 mg, 43 μL, 8.3 Eq, 0.31 mmol). The reaction mixture was stirred at 25° C. for 1 hour. The mixture was concentrated under reduced pressure. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-(4-(N-(2-(2-(2-carboxyethoxy)ethoxy)ethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid--formic acid (1/1) (20.2 mg, 14.9 μmol, 39%) as a white solid. Calc'd for C₆₃H₈₂C₁₂N₁₀O₁₉:1352.51, found [M+H−FA]]: 1307.6.

### Example 16:2,2′,2″-(10-(24-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,24-dioxo-6,9,12,15,18,21-hexaoxa-3-azatetracosyl)-1,4,7,10-tetraazacyclododecane-1,4,7-triyl)triacetic acid (Compound 16)

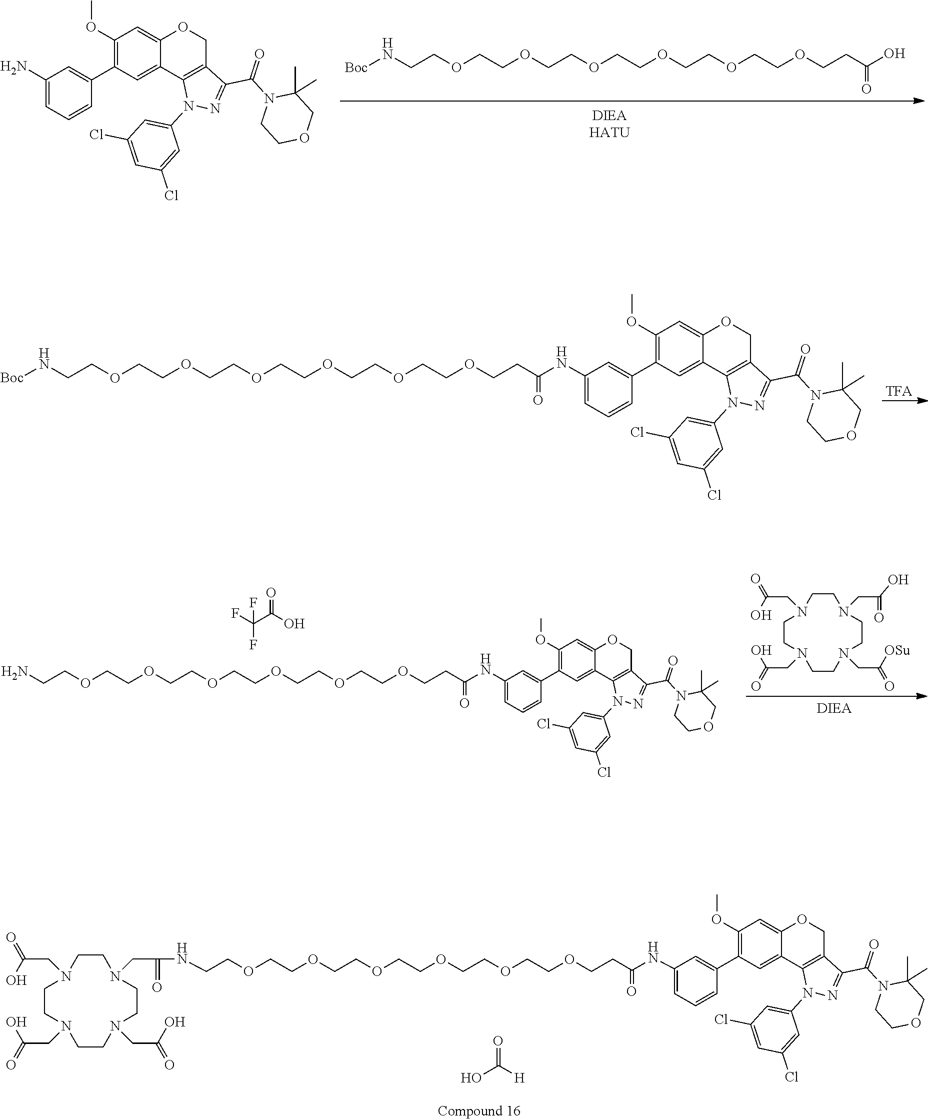

Step 1: Into an 8-mL vial, was placed a mixture of 2,2-dimethyl-4-oxo-3,8,11,14,17,20,23-heptaoxa-5-azahexacosan-26-oic acid (95 mg, 1.2 Eq, 0.21 mmol), HATU (146 mg, 2.23 Eq, 384 μmol), DIEA (68 mg, 92 μL, 3.0 Eq, 0.53 mmol) and DMF (1 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1 Eq, 173 μmol)_{w} as added and the reaction mixture was stirred at 25° C. for an additional 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(21-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-21-oxo-3,6,9,12,15,18-hexaoxahenicosyl) carbamate (115 mg, 113 μmol, 65.7%) as a yellow oil. Calc'd for C₅₀H₆₅C₁₂N₅O₁₃:1013.40, found [M+H]⁺: 1014.2.

Step 2: Into an 8-mL vial, was placed a mixture of tert-butyl(21-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-21-oxo-3,6,9,12, 15, 18-hexaoxahenicosyl) carbamate (105 mg, 1 Eq, 103 μmol) and DCM (1.0 mL), to which was added TFA (0.1 mL). The reaction mixture was stirred at 25° C. for 30 min. The mixture was concentrated under reduced pressure to provide 1-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,6,9, 12, 15, 18-hexaoxahenicosan-21-amide 2,2,2-trifluoroacetate as a yellow oil. Calc'd for C₄₇H₅₈C₁₂F₃N₅O₁₃:1027.34, found [M+H−TFA]⁺: 914.2.

Step 3: Into a 100-mL vial, was placed a mixture of 1-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,6,9,12,15,18-hexaoxahenicosan-21-amide (52 mg, 1 Eq, 57 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (88 mg, 3.1 Eq, 0.18 mmol), DIEA (46 mg, 62 μL, 6.3 Eq, 0.36 mmol), and DMF (1.0 mL). The reaction mixture was stirred at 25° C. for 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(24-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,24-dioxo-6,9,12,15,18,21-hexaoxa-3-azatetracosyl)-1,4,7,10-tetraazacyclododecane-1,4,7-triyl)triacetic acid--formic acid (1/1) (34.7 mg, 25.8 μmol, 45%) as an off-white solid. Calc'd for C₆₂H₈₅C₁₂N₉O₂₀:1345.53, found [M+H−FA]]: 1300.8.

### Example 17:2,2′,2″-(10-(18-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-2,18-dioxo-6,9,12,15-tetraoxa-3-azaoctadecyl)-1,4,7,10-tetraazacyclododecane-1,4,7-triyl)triacetic acid (Compound 17)

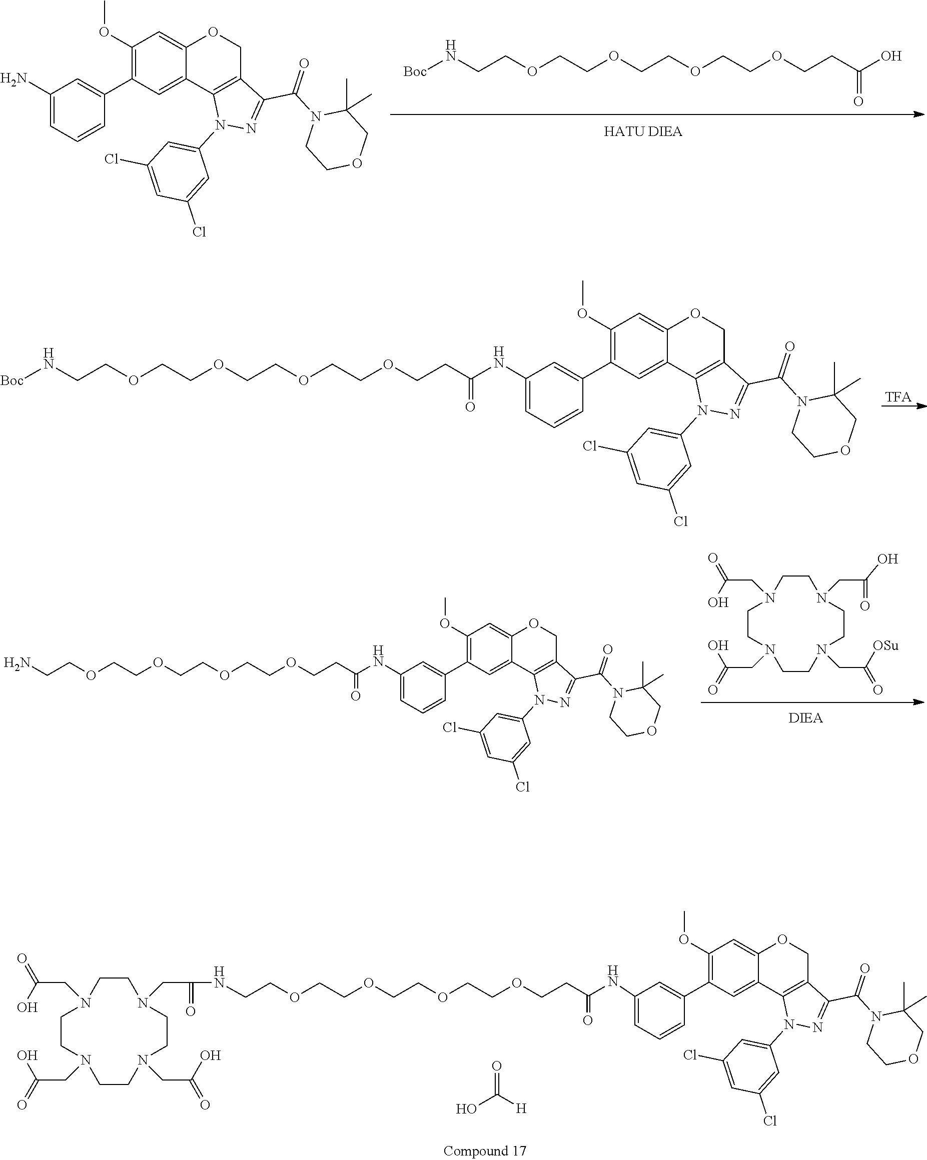

Step 1: Into a 40-mL vial, was placed a mixture of 2,2-dimethyl-4-oxo-3,8,11,14,17-pentaoxa-5-azaicosan-20-oic acid (126 mg, 2.00 Eq, 345 μmol), HATU (144 mg, 2.19 Eq, 379 μmol), DIEA (134 mg, 181 μL, 6.01 Eq, 1.04 mmol) and DMF (1.0 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1 Eq, 173 μmol)_{w} as added and the reaction mixture was stirred at 25° C. for an additional 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(15-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-15-oxo-3,6,9,12-tetraoxapentadecyl) carbamate (135 mg, 146 μmol, 84.4%) as a yellow oil. Calc'd for C₄₆H₅₇Cl₂N₅O₁₁: 925.34, found [M+H]⁺: 926.2.

Step 2: Into an 8-mL vial, was placed a mixture of tert-butyl(15-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-15-oxo-3,6,9,12-tetraoxapentadecyl) carbamate (125 mg, 1 Eq, 135 μmol) and DCM (2.0 mL), to which was added TFA (0.1 mL). The reaction mixture was stirred at 25° C. for 40 min. The mixture was diluted with water (2 mL), extracted with EtOAc (2 mL×3), then the combined organic layers were washed with water (2 mL×2) and brine (2 mL), dried over anhydrous Na₂SO₄ and concentrated under reduced pressure to provide 1-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,6,9,12-tetraoxapentadecan-15-amide (110 mg, 133 μmol, 98.7%) as a white solid. Calc'd for C₄₁H₄₉C₁₂N₅O₉:825.29, found [M+H]: 826.3. Step 3: Into an 8-mL vial, was placed a mixture of 2,2′,2″-(10-(2-((2,5-dioxopyrrolidin-1-yl)oxy)-2-oxoethyl)-1,4, 7,10-tetraazacyclododecane-1,4,7-triyl)triacetic acid (206 mg, 3.09 Eq, 411 μmol), DIEA (103 mg, 139 μL, 5.99 Eq, 797 μmol) and DMF (1.0 mL). The reaction mixture was stirred at 20° C. for 10 minutes, then 1-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,6,9,12-tetraoxapentadecan-15-amide (110 mg, 1 Eq, 133 μmol)_{w} as added and the reaction mixture was stirred at 25° C. for an additional 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(18-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,18-dioxo-6,9,12, 15-tetraoxa-3-azaoctadecyl)-1,4,7,10-tetraazacyclododecane-1,4,7-triyl)triacetic acid--formic acid (1/1) (37.8 mg, 30.0 μmol, 22.6%) as an off-white solid. Calc'd for C₅₈H₇₇C₁₂N₉O₁₈:1257.48, found [M+H−FA]⁺: 1212.6.

### Example 18:2,2′,2″-(10-(15-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-2,15-dioxo-6,9,12-trioxa-3-azapentadecyl)-1,4,7,10-tetraazacyclododecane-1,4,7-triyl)triacetic acid (Compound 18)

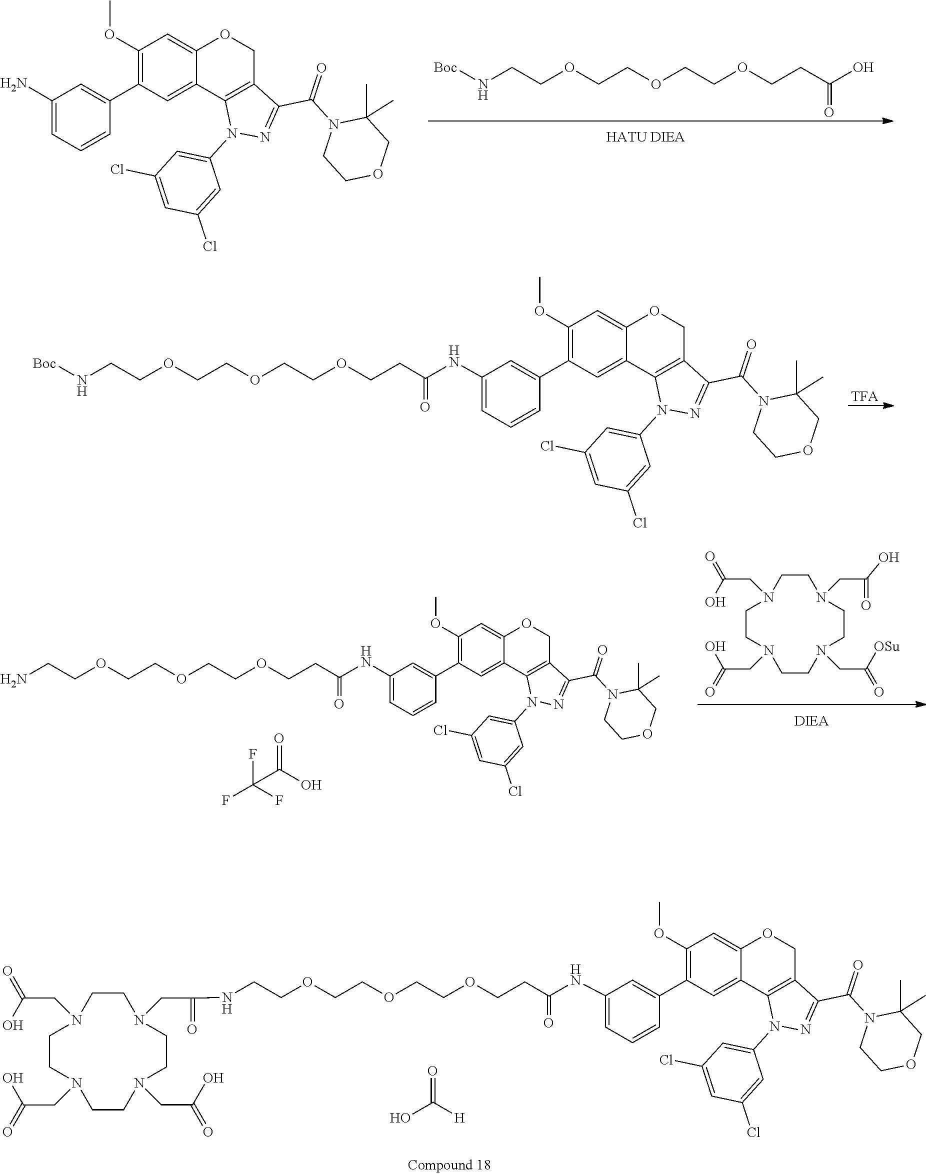

Step 1: Into an 8-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1 Eq, 173 μmol), HATU (102 mg, 1.55 Eq, 268 μmol), DIEA (76.8 mg, 104 μL, 3.44 Eq, 594 μmol) and DMF (1.0 mL). The reaction mixture was stirred at 20° C. for 10 minutes, then 2,2-dimethyl-4-oxo-3,8,11,14-tetraoxa-5-azaheptadecan-17-oic acid (112 mg, 2.02 Eq, 349 μmol)_{w} as added and the reaction mixture was stirred at 25° C. for an additional 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(2-(2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethoxy)ethyl) carbamate (105 mg, 119 μmol, 68.9%) as a yellow oil. Calc'd for C₄₄H₅₃C₁₂N₅O₁₀:881.32, found [M+H]: 882.2.

Step 2: Into an 8-mL vial, was placed a mixture of tert-butyl(2-(2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethoxy)ethyl) carbamate (95 mg, 1 Eq, 0.11 mmol) and DCM (2.0 mL), to which was added TFA (0.1 mL). The mixture was concentrated under reduced pressure to provide 3-(2-(2-(2-aminoethoxy)ethoxy)ethoxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide 2,2,2-trifluoroacetate (63 mg, 70 μmol, 65%) as a yellow oil. Calc'd for C₄₁H₄₆C₁₂F3N₅O₁₀:895.26, found [M+H−TFA]⁺: 782.1.

Step 3: Into an 8-mL vial, was placed a mixture of 3-(2-(2-(2-aminoethoxy)ethoxy)ethoxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (58 mg, 1 Eq, 74 μmol), DIEA (67 mg, 90 μL, 7.0 Eq, 0.52 mmol) and DMF (1.0 mL). The reaction mixture was stirred at 25° C. for 2 hours. The mixture was concentrated under reduced pressure. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(15-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,15-dioxo-6,9,12-trioxa-3-azapentadecyl)-1,4,7,10-tetraazacyclododecane-1,4,7-triyl)triacetic acid--formic acid (1/1) (35.0 mg, 28.8 μmol, 39%) as an off-white solid. Calc'd for C₅₆H₇₃C₁₂N₉O₁₇:1213.45, found [M+H−FA]⁺: 1168.5.

### Example 19:2,2′,2″-(10-(2-((2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl)amino)-2-oxoethyl)-1,4,7,10-tetraazacyclododecane-1,4,7-triyl)triacetic acid (Compound 19)

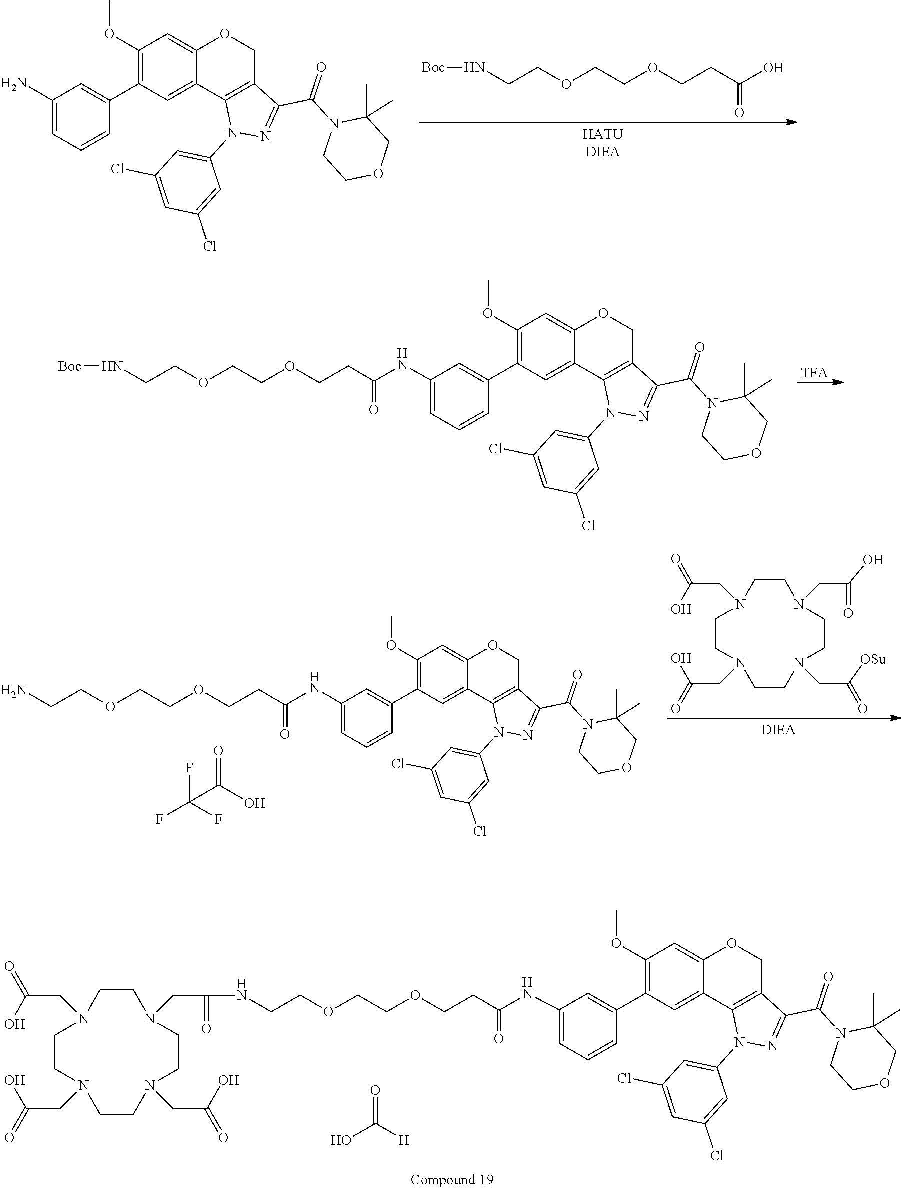

Step 1: Into an 8-mL vial, was placed a mixture of 2,2-dimethyl-4-oxo-3,8,11-trioxa-5-azatetradecan-14-oic acid (98.6 mg, 2.06 Eq, 356 μmol), HATU (145 mg, 2.21 Eq, 381 μmol), DIEA (69 mg, 93 μL, 3.1 Eq, 0.53 mmol) and DMF (1.0 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1 Eq, 173 μmol)_{w} as added and the reaction mixture was stirred at 25° C. for an additional 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamate (113 mg, 135 μmol, 78.1%) as a yellow oil. Calc'd for C₄₂H₄₉C₁₂N₅O₉:837.29, found [M+H]: 838.2.

Step 2: Into an 8-mL vial, was placed a mixture of tert-butyl(2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamate (103 mg, 1 Eq, 123 μmol) and DCM (1.0 mL), to which was added TFA (0.1 mL). The reaction mixture was stirred at 25° C. for 30 min. The mixture was concentrated under reduced pressure to provide 3-(2-(2-aminoethoxy)ethoxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide 2,2,2-trifluoroacetate (63 mg, 74 μmol, 60%) as a yellow oil. Calc'd for C₃₉H₄₂C₁₂N₅O₉:851.23, found [M+H−TFA]⁺: 738.1.

Step 3: Into an 8-mL vial, was placed a mixture of 3-(2-(2-aminoethoxy)ethoxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (63 mg, 1 Eq, 85 μmol), DIEA (68 mg, 92 μL, 6.2 Eq, 0.53 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (140 mg, 3.3 Eq, 279 μmol) and DMF (1.0 mL). The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-((2-(2-(3-((3-(1-(3-chlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl)amino)-2-oxoethyl)-1,4,7,10-tetraazacyclododecane-1,4,7-triyl)triacetic acid--formic acid (1/1) (53.0 mg, 46.6 μmol, 55%) as an off-white solid. Calc'd for C₅₄H₆₉ClN₉O₁₆:1169.42, found [M+H−FA]: 1124.5.

### Example 20:2,2′,2″-(10-(2-((2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethyl)amino)-2-oxoethyl)-1,4,7,10-tetraazacyclododecane-1,4,7-triyl)triacetic acid (Compound 20)

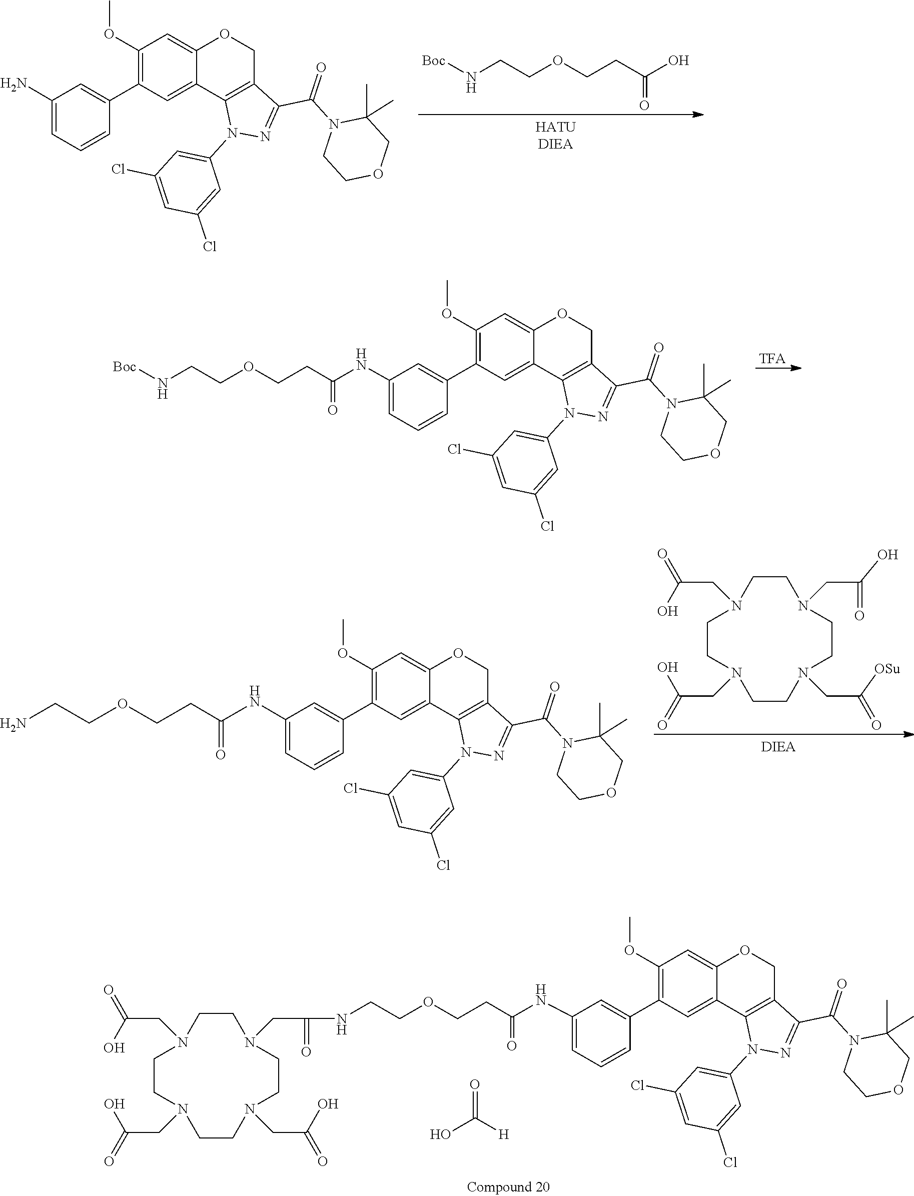

Step 1: Into an 8-mL vial, was placed a mixture of 3-(2-((tert-butoxycarbonyl)amino) ethoxy) propanoic acid (88.6 mg, 2.2 Eq, 380 μmol), HATU (131 mg, 2.0 Eq, 345 μmol), DIEA (134 mg, 180 μL, 6.0 Eq, 1.04 mmol) and DMF (1.0 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1 Eq, 173 μmol)_{w} as added and the reaction mixture was stirred at 25° C. for an additional 2 hours. The mixture was directly purified by MPLC with the following conditions: Column, WelFlash™, C18 120 g, Spherical 20-40 μm; Mobile phase, water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethyl) carbamate (118 mg, 148 μmol, 86.0%) as a yellow oil. Calc'd for C₄₀H₄₅Cl2N₅Ox: 793.27, found [M+H]: 794.2.

Step 2: Into an 8-mL vial, was placed a mixture of tert-butyl(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethyl) carbamate (108 mg, 1 Eq, 136 μmol) and DCM (2.0 mL), to which was added TFA (0.1 mL). The reaction mixture was stirred at 25° C. for 40 mins. The mixture was diluted with water (2 mL), extracted with EtOAc (50 mL×3), then the combined organic layers were washed with water (5 mL×2) and brine (2 mL), dried over anhydrous Na₂SO₄ and concentrated under reduced pressure. Purification provided 3-(2-aminoethoxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (75 mg, 0.11 mmol, 79%) as a white solid. Calc'd for C₃₅H₃₇C₁₂N₅O₆:693.21, found [M+H]: 694.2.

Step 3: Into an 8-mL vial, was placed a mixture of 2,2′,2″-(10-(2-((2,5-dioxopyrrolidin-1-yl)oxy)-2-oxoethyl)-1,4,7,10-tetraazacyclododecane-1,4,7-triyl)triacetic acid (0.15 g, 3.0 Eq, 0.30 mmol), DIEA (78 mg, 0.11 mL, 6.0 Eq, 0.60 mmol) and DMF (1.0 mL). The reaction mixture was stirred at 20° C. for 10 minutes, then 3-(2-aminoethoxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (70 mg, 1 Eq, 0.10 mmol)_{w} as added and the reaction mixture was stirred at 25° C. for an additional 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-((2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethyl)amino)-2-oxoethyl)-1,4,7,10-tetraazacyclododecane-1,4,7-triyl)triacetic acid--formic acid (1/1) as an off-white solid. Calc'd for C₅₂H₆₅C₁₂N₉O₁₅:1125.39, found [M+H−FA]⁺: 1080.5.

### Example 21:2,2′,2″-(10-(2-((((2S,3R,4R,5R,6S)-6-((2-(2-(3-((3-(1-(3,5-dichlorophenyl)-3-(3,3-dimethylmorpholine-4-carbonyl)-7-methoxy-1,4-dihydrochromeno|4,3-clpyrazol-8-yl)phenyl)amino)-3-oxopropoxy)ethoxy)ethyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid (Compound 21)

### Synthesis of Intermediate-A

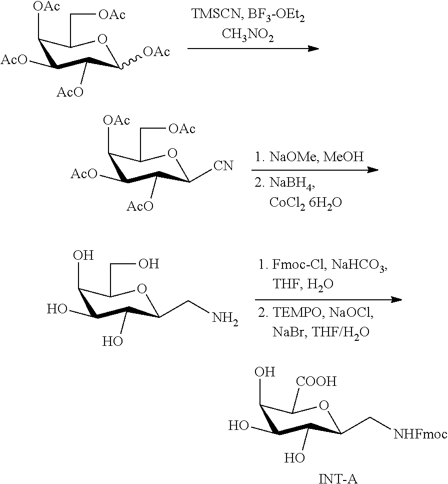

Step 1: Synthesis of (2R,3S,4R,5S,6S)-2-(acetoxymethyl)-6-cyanotetrahydro-2H-pyran-3,4,5-triyl triacetate. To a stirred solution of (3R,4S,5S,6R)-6-(acetoxymethyl)tetrahydro-2H-pyran-2,3,4,5-tetrayl tetraacetate (5.0 g, 11.47 mmol, 1.0 eq.) in nitromethane (25 mL) at 0° C. was added TMSCN (2.15 g, 22.1 mmol, 1.7 eq) and BF3·OEt2 (0.65 g, 4.66 mmol, 0.36 eq) and the resultant reaction mixture was stirred at room temperature for 1 h. Then, TMSCN (2.15 g, 22.1 mmol, 1.7 eq) and BF3.OEt2 (0.65 g, 4.66 mmol, 0.36 eq) were added and the reaction mixture was stirred at room temperature for 3 h. Upon completion, the reaction mixture was concentrated under vacuum to get a crude product, which was dissolved in EtOAc and washed with a saturated solution of NaHCO₃, water and brine. The combined organic layer was dried over anhydrous Na₂SO₄ filtered and concentrated in vacuo to afford title (2R,3S,4R,5S,6S)-2-(acetoxymethyl)-6-cyanotetrahydro-2H-pyran-3,4,5-triyl triacetate as a brown solid (3.0 g, 65%).

Step 2: Synthesis of (2S,3R,4R,5R,6R)-3,4,5-trihydroxy-6-(hydroxymethyl)tetrahydro-2H-pyran-2-carbonitrile (3). To a stirred solution of (2R,3S,4R,5S,6S)-2-(acetoxymethyl)-6-cyanotetrahydro-2H-pyran-3,4,5-triyl triacetate (11.0 g, 30.81 mmol, 1.0 eq.) in methanol (1300 mL) at −10° C. was added NaOMe (1M in MeOH, 4.4 mL) and the resultant reaction mixture was stirred at −10° C. for 1 h. Upon completion of the reaction, the reaction mixture was acidified with amberlite IR-120 (H+resin) to pH˜6-7 at 0° C. The reaction mixture was filtered and the filtrate was concentrated in vacuo to afford 2S,3R,4R,5R,6R)-3,4,5-trihydroxy-6-(hydroxymethyl)tetrahydro-2H-pyran-2-carbonitrile as a brown liquid (5.5 g, 94%).

Step 3: Synthesis of (2S,3R,4R,5R,6R)-2-(aminomethyl)-6-(hydroxymethyl)tetrahydro-2H-pyran-3,4,5-triol. To a stirred solution of 2S,3R,4R,5R,6R)-3,4,5-trihydroxy-6-(hydroxymethyl)tetrahydro-2H-pyran-2-carbonitrile (4 g, 21.16 mmol, 1.0 eq.) in THF and (32 mL)_{w}ater (16 mL) at 0° C. was added NaBH₄ (1.6 g, 42.35 mmol, 2.0 eq). The resultant reaction mixture was stirred at room temperature for 1 h. Then, additional NaBH4 (1.6 g, 42.35 mmol, 2.0 eq)_{w} as added and the reaction mixture was stirred at room temperature for 2 h. Upon completion, the reaction mixture was quenched with 28% NH₄OH and filtered. The filtrate was concentrated in vacuo to get a crude product. The obtained crude compound was purified by column chromatography over Dowex (H″ ion exchange resin) with a gradient elution of 0-5% NH₄OH to afford (2S,3R,4R,5R,6R)-2-(aminomethyl)-6-(hydroxymethyl)tetrahydro-2H-pyran-3,4,5-triol as a pale-yellow solid (1.65 g, 40% yield). Calc'd for C₇H₁₅O₅:193.1, found [M+H]: 194.

Step 4: Synthesis of (9H-fluoren-9-yl)methyl (((2S,3R,4R,5R,6R)-3,4,5-trihydroxy-6-(hydroxymethyl)tetrahydro-2H-pyran-2-yl)methyl) carbamate. To a stirred solution of (2S,3R,4R,5R,6R)-2-(aminomethyl)-6-(hydroxymethyl)tetrahydro-2H-pyran-3,4,5-triol (3.0 g, 15.54 mmol, 1.0 eq) in THF (60 mL) and 10% NaHCO₃ at 0° C. was added a solution of Fmoc-C₁ (4.0 g, 15.54 mmol, 1.0 eq). The resultant reaction mixture was stirred at room temperature for 3 h. Upon completion, the reaction mixture was diluted with water and acidified with conc. HCl, (pH˜3 to 4) then concentrated to get a crude product. The crude compound was purified by reverse phase column chromatography (Combi flash) with a gradient elution of 0-50% acetonitrile in water to afford (9H-fluoren-9-yl)methyl (((2S,3R,4R,5R,6R)-3,4,5-trihydroxy-6-(hydroxymethyl)tetrahydro-2H-pyran-2-yl)methyl) carbamate as an off-white solid (1.0 g, 15.5% yield). Calc'd for C₂₂H₂₅NO₇:415.16, found [M+H]: 416.

Step 5: Synthesis of (2S,3R,4R,5R,6S)-6-(((((9H-fluoren-9-yl) methoxy) carbonyl)amino)methyl)-3,4,5-trihydroxytetrahydro-2H-pyran-2-carboxylic acid (INT-A). To a stirred solution of (9H-fluoren-9-yl)methyl (((2S,3R,4R,5R,6R)-3,4,5-trihydroxy-6-(hydroxymethyl)tetrahydro-2H-pyran-2-yl)methyl) carbamate (2 g, 1.22 mmol, 1.0 eq) in THF (40 mL) and water (40 mL) at 0° C. was added NaHCO₃ (2.4 g, 29 mmol, 6.0 eq), TEMPO(225 mg, 1.44 mmol, 0.3 eq) and NaBr (240 mg, 2.44 mmol, 0.5 eq) followed by NaOCI (10-12%) (12 mL, 14.52 mmol, 3.0 eq). The resultant reaction mixture was stirred at room temperature for 3 h. Upon completion, the reaction mixture was concentrated and extracted with Et₂O(2×150 mL), then the aqueous layer was acidified with conc. HCl to pH 2-3 and extracted with ethyl acetate (2×200 mL). The combined organic layer was washed with brine (10 mL), dried over anhydrous Na₂SO₄ and concentrated under reduced pressure to provide the crude compound. The crude compound was purified by reverse phase column chromatography using 0-70% gradient elution of acetonitrile to afford the title compound INT-A as an off-white solid (1.3 g, 63% yield). Calc'd for C₂₂H₂₃NO₈:429.14, found [M+H]: 430.

### Synthesis of Compound 21

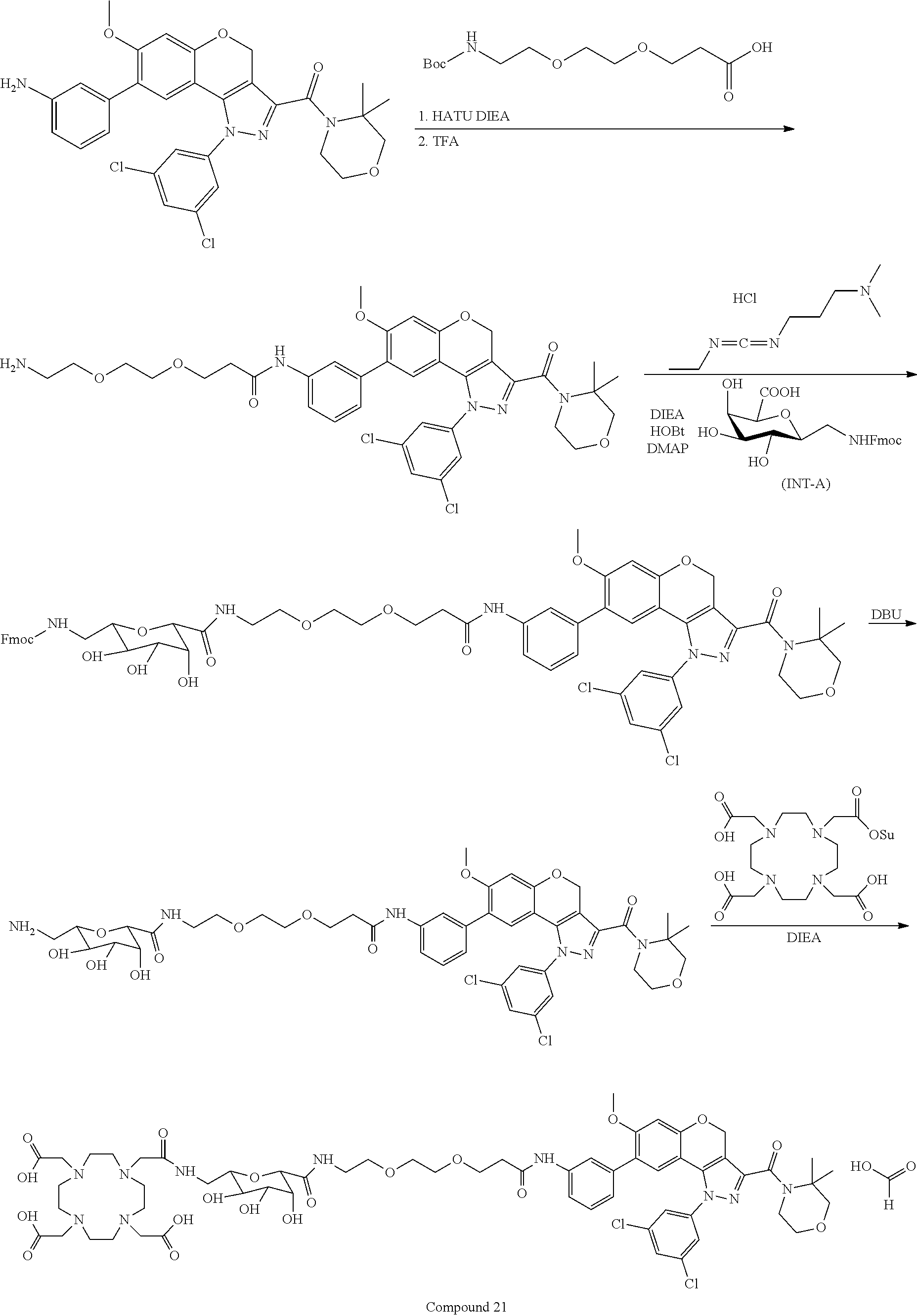

Step 1: Into an 8-mL vial, was placed a mixture of 2,2-dimethyl-4-oxo-3,8,11-trioxa-5-azatetradecan-14-oic acid (110 mg, 1.15 Eq, 397 μmol) and DMF (2 mL), to which was added HATU (190 mg, 1.45 Eq, 500 μmol) and DIEA (140 mg, 189 μL, 3.14 Eq, 1.08 mmol). The reaction mixture was stirred at 25° C. for 20 mins, then (8-(3-aminophenyl)-1-(3,5-dichlorophenyl)-7-methoxy-1,4-dihydrochromeno[4,3-c]pyrazol-3-yl) (3,3-dimethylmorpholino) methanone (200 mg, 1 Eq, 345 μmol)_{w} as added. The reaction mixture was stirred at 25° C. for 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 9 min, 98% ACN to 98% in 1 min); Total flow rate, 70 mL/min; Detector, UV 220 nm. The collected fractions were concentrated to afford tert-butyl(2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamate (220 mg, 262 μmol, 76.0%) as a yellow solid. Calc'd for C₄₂H₄₉C₁₂N₅O₉:837.29, found [M+H]: 838.3, 840.3.

Step 2: Into a 8 mL flask were added a mixture of tert-butyl(2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamate (200 mg, 1 Eq, 238 μmol), DCM (2 mL) and TFA (0.1 mL). The mixture was stirred for 0.5 hour at 24° C. The collected fractions were concentrated under reduced pressure to provide 3-(2-(2-aminoethoxy)ethoxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (250 mg, 0.17 mmol, 71%, 50% Purity) as brown solid. Calc'd for C₃₉H₄₂C₁₂F3N₅O₉:851.23, found [M+H−TFA]: 738.3, 740.3.

Step 3: Into an 8-mL vial, was placed a mixture of (2S,3R,4R,5R,6S)-6-(((((9H-fluoren-9-yl) methoxy) carbonyl)amino)methyl)-3,4,5-trihydroxytetrahydro-2H-pyran-2-carboxylic acid (INT-A, 150 mg, 1.29 Eq, 349 μmol) and DMF (2 mL), to which was added 3-(((ethylimino)methylene)amino)-N,N-dimethylpropan-1-amine hydrochloride (65 mg, 1.3 Eq, 0.34 mmol), N-ethyl-N-isopropylpropan-2-amine (110 mg, 3.14 Eq, 851 μmol), 1H-benzo[d][1,2,3]triazol-1-ol hydrate (50 mg, 1.2 Eq, 0.33 mmol), and N,N-dimethylpyridin-4-amine (20 mg, 0.60 Eq, 0.16 mmol). The reaction mixture was stirred at 26° C. for 20 mins, then 3-(2-(2-aminoethoxy)ethoxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (200 mg, 1 Eq, 271 μmol)_{w} as added. The reaction mixture was stirred at 26° C. for 3 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated to afford (9H-fluoren-9-yl)methyl (((2S,3R,4R,5R,6S)-6-((2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamoyl)-3,4,5-trihydroxytetrahydro-2H-pyran-2-yl)methyl) carbamate (250 mg, 87 μmol, 32%, 40% Purity) as a yellow solid. Calc'd for C₅₉H₆₂C₁₂N₆O₁₄:1148.37, found [M+H]⁺: 1149.3, 1151.3.

Step 4: Into an 8-mL vial, was placed a mixture of (9H-fluoren-9-yl)methyl (((2S,3R,4R,5R,6S)-6-((2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamoyl)-3,4,5-trihydroxytetrahydro-2H-pyran-2-yl)methyl) carbamate (230 mg, 40% Wt, 1 Eq, 80.0 μmol) and DMF (2 mL), to which was added 2,3,4,6,7,8,9,10-octahydropyrimido[1,2-a]azepine (65 mg, 5.3 Eq, 0.43 mmol). The reaction mixture was stirred at 27° C. for 3 hours. The crude product was purified by Prep-HPLC with the following conditions: Column: SunFire prep OBD 19*150 mm 5 μm; Mobile Phase A: Water (0.1% TFA); Mobile Phase B: ACN; Gradient: 25% B to 65% B in 10 min; Flow rate: 20 mL/min; Wave Length: 220 nm. The collected fractions were dried by lyophilization to afford (2S,3R,4R,5R,6S)-6-(aminomethyl)-N-(2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl)-3,4,5-trihydroxytetrahydro-2H-pyran-2-carboxamide (60 mg, 65 μmol, 81%) as a white solid. Calc'd for C₄₄H₅₂C₁₂N₆O₁₂:926.30, found [M+H]: 927.3, 929.3.

Step 5: Into an 8-mL vial, was placed a mixture of (2S,3R,4R,5R,6S)-6-(aminomethyl)-N-(2-(2-(3-((3-(1-(3,5-dichlorophenyl)-3-(3,3- dimethylmorpholine-4-carbonyl)-7-methoxy-1,4-dihydrochromeno[4,3-c]pyrazol-8-yl)phenyl)amino)-3-oxopropoxy)ethoxy)ethyl)-3,4,5-trihydroxytetrahydro-2H-pyran-2-carboxamide (60 mg, 1 Eq, 65 μmol) and DMF (1 mL), to which was added 2,2′,2″-(10-(2-((2,5-dioxopyrrolidin-1-yl)oxy)-2-oxoethyl)-1,4,7,10-tetraazacyclododecane-1,4,7-triyl)triacetic acid (95 mg, 2.9 Eq, 0.19 mmol), DIEA (45 mg, 61 μL, 5.4 Eq, 0.35 mmol). The reaction mixture was stirred at 24° C. for 16 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 10 min; Flow rate: 20 mL/min; Wave Length: 220 nm. The collected fractions were dried by lyophilization to provide 2,2′,2″-(10-(2-((((2S,3R,4R,5R,6S)-6-((2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid--formic acid (1/1) (51.4 mg, 37.8 μmol, 58%) as a white solid. Calc'd for C₆₁H₈₀C₁₂N₁₀O₂₁:1358.49, found [M+H−FA]⁺: 1313.6, 1315.6.

### Example 22:2,2′,2″-(10-(1-((3-(1-(3,5-dichlorophenyl)-3-(3,3-dimethylmorpholine-4-carbonyl)-7-methoxy-1,4-dihydrochromeno|4,3-c]pyrazol-8-yl)phenyl)amino)-3,6,9,12,15-pentamethyl-1,4,7,10,13,16,20-heptaoxo-3,6,9,12,15,19-hexaazahenicosan-21-yl)-1,4,7,10-tetraazacyclododecane-1,4,7-triyl)triacetic acid (Compound 22)

### Synthesis of 1-(9H-fluoren-9-yl)-8,11,14,17,20-pentamethyl-3,7,10,13,16,19-hexaoxo-2-oxa-4,8,11,14,17.20-hexaazadocosan-22-oic acid (Intermediate-B)

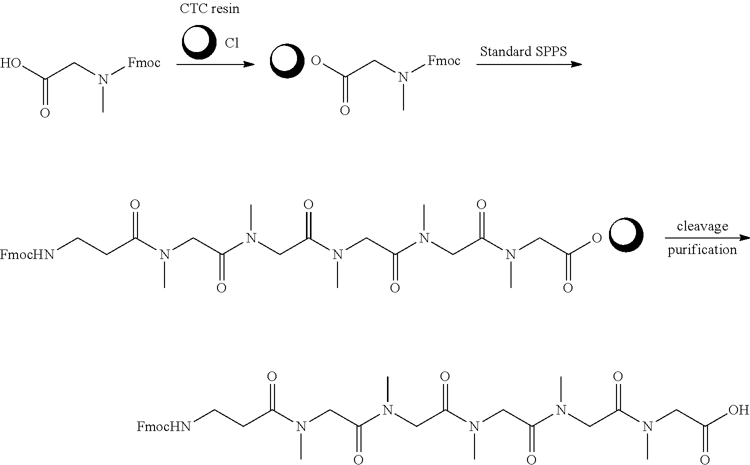

Step 1:15 g of Fmoc-Sar-OH was attached onto 36 g of CTC resin (1.1 mmol/g resin) [General procedure step 1].

Step 2: Elongation was finished using standard solid phase peptide synthesis(SPPS) [General procedure step 3 and 4].

Step 3: The peptide was cleaved from the resin following General procedure step 6B.

Step 4: The residue was purified by reversed-phase flash chromatography with the following conditions: column, C18; mobile phase, MeCN in Water, 28% to 45% gradient in 20 min; detector, UV 254 nm. The collected fractions were concentrated under reduced pressure to afford product as a white solid (5.1183 g, overall yield 15.74%, 98.5% purity). Calc'd for C₇₈H₁₀₅C₁₂N₁₇O₂₄:666.3, found [M+H]⁺: 667.2.

### Synthesis of Compound 22

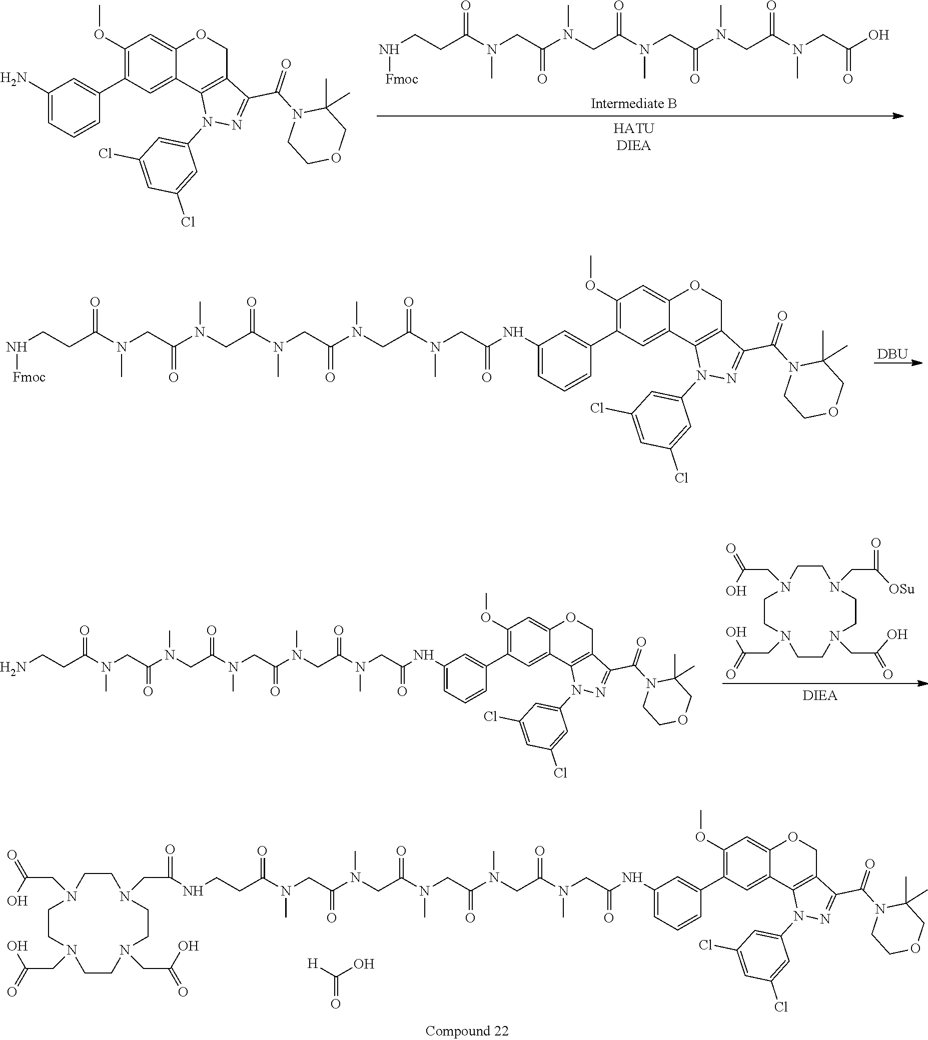

Step 1: Into an 8-mL vial, was placed a mixture of 1-(9H-fluoren-9-yl)-8,11,14,17,20-pentamethyl-3,7,10,13,16, 19-hexaoxo-2-oxa-4,8,11,14, 17,20-hexaazadocosan-22-oic acid (Intermediate-B, 140 mg, 1.22 Eq, 210 μmol), HATU (100 mg, 1.52 Eq, 263 μmol), DIEA (70 mg, 94 μL, 3.1 Eq, 0.54 mmol) and DMF (1 mL). The reaction mixture was stirred at 28° C. for 10 mins, then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1 Eq, 173 μmol)_{w} as added. The reaction mixture was stirred at 28° C. for 1 hour. The collected fractions were concentrated under reduced pressure to provide (9H-fluoren-9-yl)methyl (1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,6,9,12,15-pentamethyl-1,4,7,10,13, 16-hexaoxo-3,6,9,12,15-pentaazaoctadecan-18-yl) carbamate (160 mg, 78 μmol, 45%, 60% Purity) as a yellow solid. Calc'd for C₆₃H₆₈C₁₂N₁₀O₁₂:1226.44, found [M+H]⁺: 1227.4, 1229.4.

Step 2: Into an 8-mL vial, was placed a mixture of (9H-fluoren-9-yl)methyl (1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,6,9,12, 15-pentamethyl-1,4,7, 10, 13, 16-hexaoxo-3,6,9, 12, 15-pentaazaoctadecan-18-yl) carbamate (160 mg, 1 Eq, 130 μmol) and DMF (2 mL), to which was added 2,3,4,6,7,8,9,10-octahydropyrimido[1,2-a]azepine (150 mg, 7.56 Eq, 985 μmol). The reaction mixture was stirred at 50° C. for 3 hours. The crude product was purified by Prep-HPLC using the following conditions: Column: SunFire prep OBD 19*150 mm 5 μm; Mobile Phase A: Water (0.1% TFA); Mobile Phase B: ACN; Gradient: 25% B to 65% B in 10 min; Flow rate: 20 mL/min; Wave Length: 220 nm. The collected fractions were dried by lyophilization. Purification provided 3-amino-N-(14-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,6,9,12-tetramethyl-2,5,8,11, 14-pentaoxo-3,6,9,12-tetraazatetradecyl)-N-methylpropanamide (80 mg, 80 μmol, 61%) as a white solid. [M+H]=1005.3, 1007.3. Calc'd for C₄₈H₅₈C₁₂N₁₀O₁₀:1004.37, found [M+H]: 1005.3, 1007.3.

Step 3: Into an 8-mL vial, was placed a mixture of 3-amino-N-(14-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,6,9,12-tetramethyl-2,5,8,11,14-pentaoxo-3,6,9,12-tetraazatetradecyl)-N-methylpropanamide (75 mg, 1 Eq, 75 μmol) and DMF (1 mL), to which was added N-ethyl-N-isopropylpropan-2-amine (70 mg, 7.3 Eq, 0.54 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (80 mg, 2.1 Eq, 0.16 mmol). The reaction mixture was stirred at 28° C. for 2 hours. The crude product was purified by Prep-HPLC using the following conditions: Column: SunFire prep OBD 19*150 mm 5 μm; Mobile Phase A: Water (0.1% FA); Mobile Phase B: ACN; Gradient: 25% B to 65% B in 10 min; Flow rate: 20 mL/min; Wave Length: 220 nm. The collected fractions were dried by lyophilization to provide 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,6,9, 12, 15-pentamethyl-1,4,7,10, 13, 16,20-heptaoxo-3,6,9,12, 15, 19-hexaazahenicosan-21-yl)-1,4, 7, 10-tetraazacyclododecane-1,4,7-triyl)triacetic acid--formic acid (1/1) (64.3 mg, 44.7 μmol, 60%) as a white solid. Calc'd for C₆₅H₈₆C₁₂N₁₄O₁₉:1436.38, found [M+H−FA]: 1391.8, 1393.8.

### Example 23: (R)-2,2′,2″-(10-(2-((5-amino-6-((2-(3-(3-(1-(3,5-dichlorophenyl)-3-(3,3-dimethylmorpholine-4-carbonyl)-7-methoxy-1,4-dihydrochromeno|4,3-c]pyrazol-8-yl)phenyl)ureido)ethyl)amino)-6-oxohexyl)amino)-2-oxoethyl)-1,4,7,10-tetraazacyclododecane-1,4,7-triyl)triacetic acid (Compound 23)

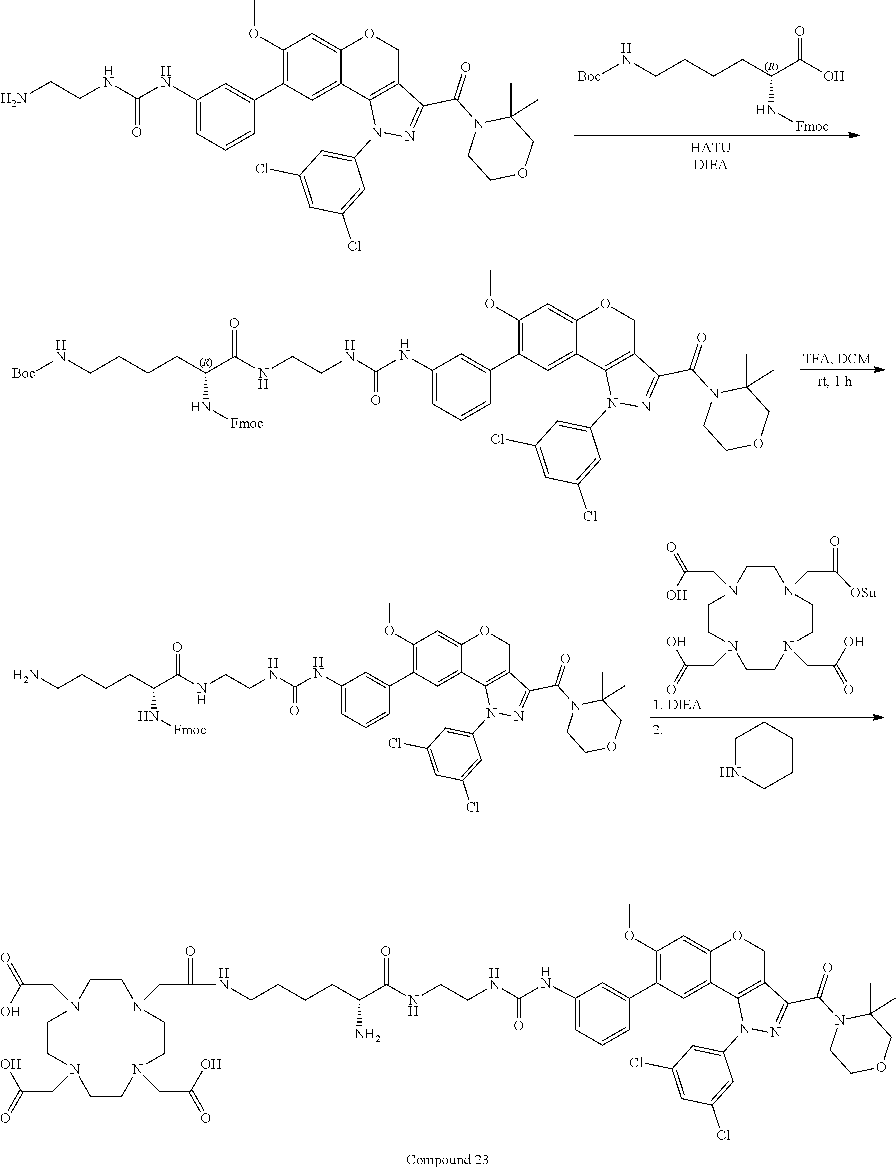

Step 1: Into a 8-mL vial, was placed a mixture of N₂-(((9H-fluoren-9-yl) methoxy) carbonyl)-N₆-(tert-butoxycarbonyl)-D-lysine (85 mg, 1.2 Eq, 0.18 mmol), HATU (98 mg, 1.7 Eq, 0.26 mmol), DIEA (60 mg, 81 μL, 3.1 Eq, 0.46 mmol) and DMF (1.0 mL). The reaction mixture was stirred at 20° C. for 10 minutes, then 1-(2-aminoethyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (100 mg, 1 Eq, 150 μmol)_{w} as added and the reaction mixture was stirred at 25° C. for an additional 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided (9H-fluoren-9-yl)methyl tert-butyl(6-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-6-oxohexane-1,5-diyl) (R)-dicarbamate (90 mg, 81 μmol, 54%) as a yellow oil. Calc'd for C₅₉H₆₄Cl2N&O10:1114.41, found [M+H]: 1115.3.

Step 2: Into an 8-mL vial, was placed a mixture of (9H-fluoren-9-yl)methyl tert-butyl (6-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-6-oxohexane-1,5-diyl) (R)-dicarbamate (90 mg, 1 Eq, 81 μmol) and DCM (4 mL), to which was added TFA (0.2 mL). The mixture was concentrated under reduced pressure to provide (9H-fluoren-9-yl)methyl (R)-(6-amino-1-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-1-oxohexan-2-yl) carbamate (65 mg, 64 μmol, 79%) as a yellow oil. Calc'd for C₅₄H₅₆Cl2N&Ox: 1014.36, found [M+H]: 1015.3.

Step 3: Into an 8-mL vial, was placed a mixture of (9H-fluoren-9-yl)methyl (R)-(6-amino-1-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-1-oxohexan-2-yl) carbamate (65 mg, 1 Eq, 64 μmol), DIEA (60 mg, 81 μL, 7.3 Eq, 0.46 mmol) and DMF (1.0 mL). The resulting mixture was used directly for the next step without any purification. Calc'd for C₇₀H₈₂C₁₂N₁₂O₁₅:1400.54, found [M+H]: 1401.4.

Step 4: Into an 8-mL vial, was placed a mixture of (R)-2,2′,2″-(10-(2-((5-((((9H-fluoren-9-yl) methoxy) carbonyl)amino)-6-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-6-oxohexyl)amino)-2-oxoethyl)-1,4,7,10-tetraazacyclododecane-1,4,7-triyl)triacetic acid (1 Eq), piperidine (82 mg, 95 μL, 1 Eq, 0.96 mmol) and DMF (1.0 mL). The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(2-((5-amino-6-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-6-oxohexyl)amino)-2-oxoethyl)-1,4,7,10-tetraazacyclododecane-1,4,7-triyl)triacetic acid as a white solid. Calc'd for C₅₅H₇₂C₁₂N₁₂O₁₃:1178.47, found [M+H]: 1179.8.

### Example 24:2,2′,2″-(10-(2-((2-((2-((2-(3-(3-(1-(3,5-dichlorophenyl)-3-(3,3-dimethylmorpholine-4-carbonyl)-7-methoxy-1,4- dihydrochromeno|4,3-c|pyrazol-8-yl)phenyl)ureido)ethyl)amino)-3,4-dioxocyclobut-1-en-1-yl)amino)ethyl)amino)-2-oxoethyl)-1,4,7,10-tetraazacyclododecane-1,4,7-triyl)triacetic acid (Compound 24)

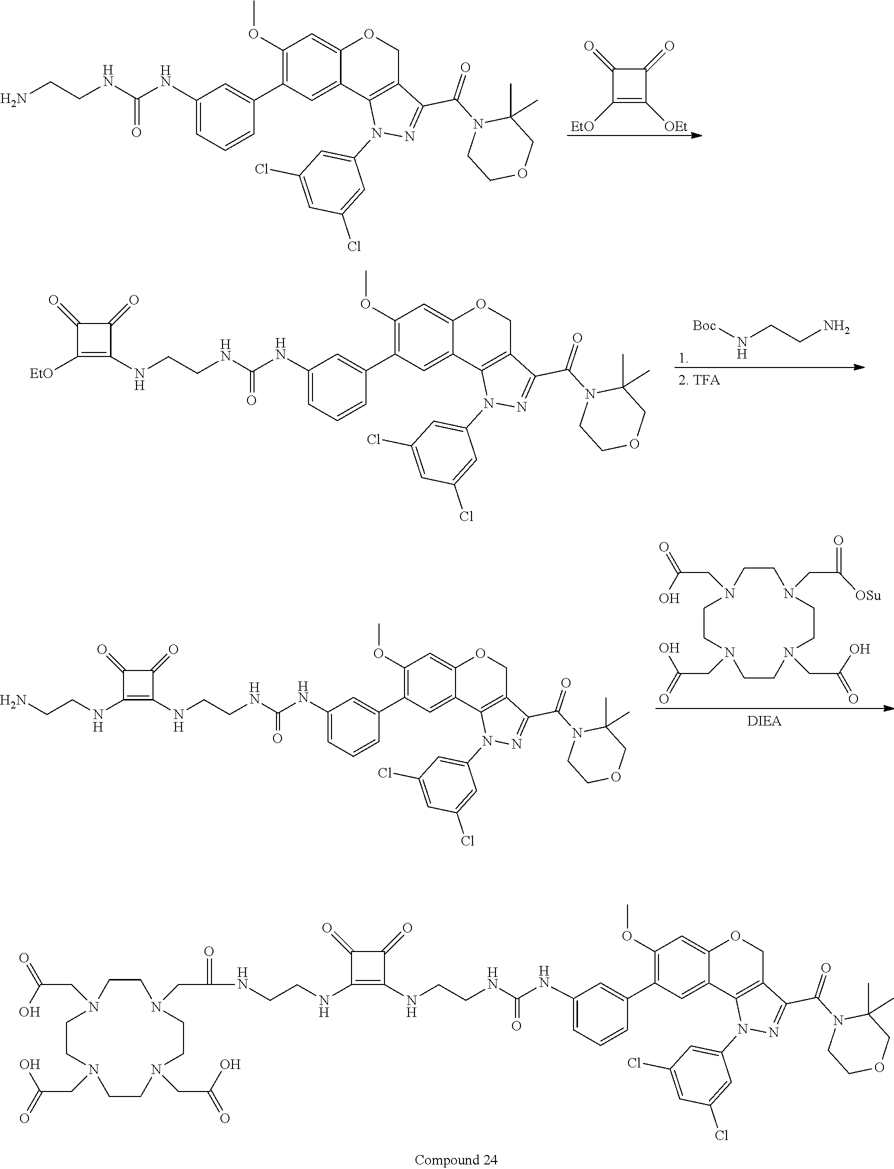

Step 1: Into an 8-mL vial, was placed a mixture of 1-(2-aminoethyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (100 mg, 1 Eq, 150 μmol), 3,4-diethoxycyclobut-3-ene-1,2-dione (53 mg, 2.1 Eq, 0.31 mmol) and PBS (1.0 mL). The reaction mixture was stirred at 25° C. for 16 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided 1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3-(2-((2-ethoxy-3,4-dioxocyclobut-1-en-1-yl)amino)ethyl)urea (60 mg, 76 μmol, 51%) as a white solid. Calc'd for C₃₉H₃₈C₁₂N₆Og: 788.21, found [M+H]⁺: 789.2.

Step 2: Into an 8-mL vial, was placed a mixture of 1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3-(2-((2-ethoxy-3,4-dioxocyclobut-1-en-1-yl)amino)ethyl)urea (80 mg, 1 Eq, 0.10 mmol), tert-butyl(2-aminoethyl) carbamate (130 mg, 8.0 Eq, 811 μmol) and PBS (1.0 mL). The reaction mixture was stirred at 25° C. for 16 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(2-((2-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,4-dioxocyclobut-1-en-1-yl)amino)ethyl) carbamate (62 mg, 69 μmol, 68%) as a white solid. Calc'd for C₄₄H₄₈C₁₂N&O9:902.29, found [M+H]: 903.20.

Step 3: Into an 8-mL vial, was placed a mixture of tert-butyl(2-((2-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,4-dioxocyclobut-1-en-1-yl)amino)ethyl) carbamate (62 mg, 1 Eq, 69 μmol) and DCM (3.0 mL), to which was added TFA (0.15 mL). The reaction mixture was stirred at 25° C. for 16 hours. The mixture was concentrated under reduced pressure to provide 1-(2-((2-((2-aminoethyl)amino)-3,4-dioxocyclobut-1-en-1-yl)amino)ethyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (46 mg, 57 μmol, 83%) as a white solid. Calc'd for C₄₄H₄₈C₁₂N&O9:802.24, found [M+H]: 803.20.

Step 4: Into an 8-mL vial, was placed a mixture of 1-(2-((2-((2-aminoethyl)amino)-3,4-dioxocyclobut-1-en-1-yl)amino)ethyl)-3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)urea (46 mg, 1 Eq, 57 μmol), N-ethyl-N-isopropylpropan-2-amine, 2,2′,2″-(10-(2-((2,5-dioxopyrrolidin-1-yl)oxy)-2-oxoethyl)-1,4,7,10-tetraazacyclododecane-1,4,7-triyl)triacetic acid (60 mg, 2.1 Eq, 0.12 mmol) and DMF (1.0 mL). The reaction mixture was stirred at 25° C. for 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to 2,2′,2″-(10-(2-((2-((2-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,4-dioxocyclobut-1-en-1-yl)amino)ethyl)amino)-2-oxoethyl)-1,4,7,10-tetraazacyclododecane-1,4,7-triyl)triacetic acid (16.3 mg, 13.7 μmol, 24%) as an off-white solid. Calc'd for C₅₅H₆₆C₁₂N₁₂O₁₄:1188.42, found [M+H]⁺: 1189.5.

### Example 25: (R)-2,2′,2″-(10-(2-((2-(2-(22-((3-(1-(3,5-dichlorophenyl)-3-(3,3-dimethylmorpholine-4-carbonyl)-7-methoxy-1,4- dihydrochromeno[4,3-c]pyrazol-8-yl)phenyl) carbamoyl)-20-oxo-2,5,8,11,14,17-hexaoxa-21-azatetracosan-24-oyl)-2,5-diazaspiro[3.4]octan-5-yl)-2-oxoethyl)amino)-2-oxoethyl)-1,4,7,10-tetraazacyclododecane-1,4,7-triyl)triacetic acid (Compound 25)

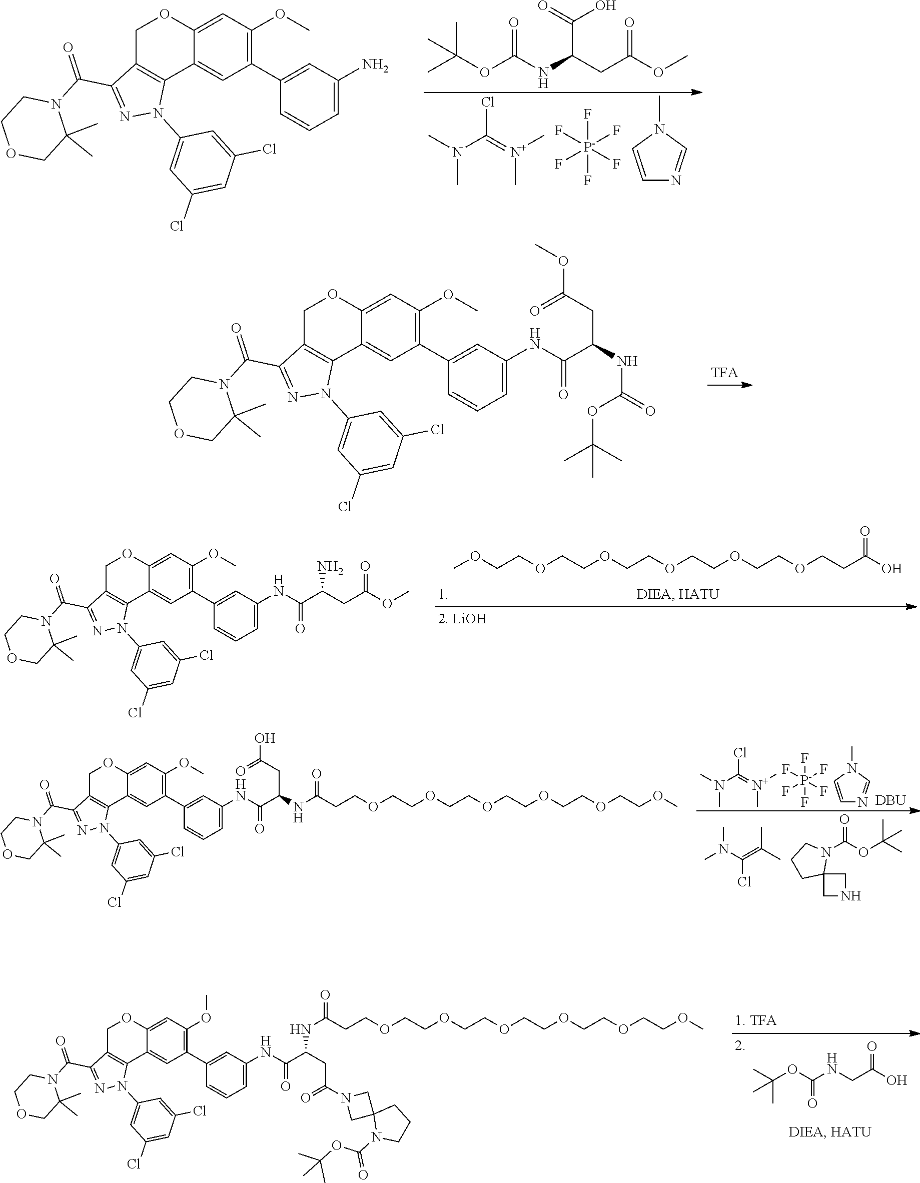

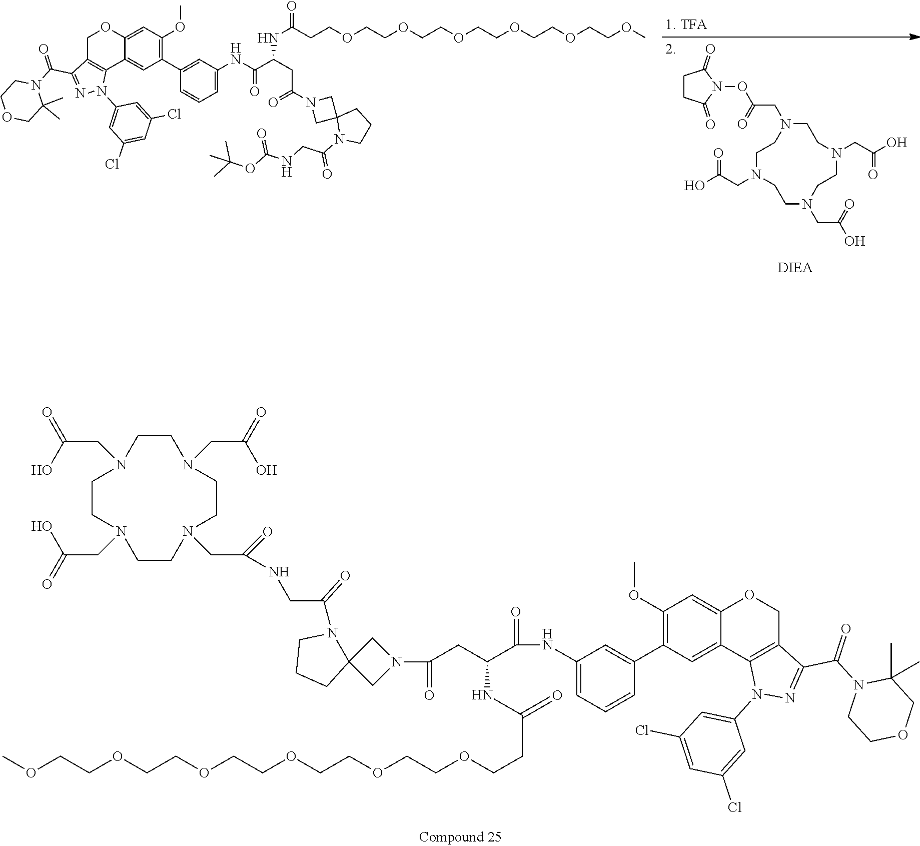

Step 1: Into an 8-mL vial, was placed a mixture of (R)-2-((tert-butoxycarbonyl)amino)-4-methoxy-4-oxobutanoic acid (21.3 mg, 1 Eq, 86.1 μmol), chloro-N,N,N′,N′-tetramethylformamidinium hexafluorophosphate (58.1 mg, 2.40 Eq, 207 μmol), 1-Methylimidazole (N—) (42.5 mg, 41.1 μL, 6.01 Eq, 518 μmol) and DMF (1 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 2.00 Eq, 173 μmol)_{w} as added and the reaction mixture was stirred at 25° C. for an additional 1 hour. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide methyl (R)-3-((tert-butoxycarbonyl)amino)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoate (117 mg, 145 μmol, 168%) as a yellow oil. Calc'd for C₄₀H₄₃C₁₂N₅O₉:807.24, found [M+H]⁺: 808.2, 810.2. Step 2: Into an 8-mL vial, was placed a mixture of methyl (R)-3-((tert-butoxycarbonyl)amino)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoate (115 mg, 1 Eq, 142 μmol) and DCM (6 mL), to which was added TFA (0.3 mL). The reaction mixture was stirred at 25° C. for 1 hour. The mixture was concentrated under reduced pressure to provide methyl (R)-3-amino-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoate (108 mg, 152 μmol, 107%) as a solid. Calc'd for C₃₅H₃₅Cl2N_{507:707.19}, found [M+H]: 708.2, 710.2.

Step 3: Into an 8-mL vial, was placed a mixture of 2,5,8,11, 14, 17-hexaoxaicosan-20-oic acid (98.9 mg, 1.00 Eq, 305 μmol), DIEA (118 mg, 159 μL, 2.99 Eq, 913 μmol), HATU (139 mg, 1.20 Eq, 366 μmol) and DMF (1.5 mL). The reaction mixture was stirred at 20° C. for 10 minutes, then methyl (R)-3-amino-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoate (108 mg, 0.5 Eq, 152 μmol)_{w} as added and the reaction mixture was stirred at 25° C. for an additional 1 hour. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide methyl (R)-22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8,11,14,17-hexaoxa-21-azatetracosan-24-oate (90 mg, 89 μmol, 29%) as a yellow solid. Calc'd for C₄₉H₆₁C₁₂N₅O₁₄:1013.36, found [M+H]: 1014.5, 1016.5, [M+Na]=1036.5, 1038.5.

Step 4: Into an 8-mL vial, was placed a mixture of methyl (R)-22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8, 11, 14,17-hexaoxa-21-azatetracosan-24-oate (90 mg, 1 Eq, 89 μmol), LiOH (11 mg, 5.2 Eq, 0.46 mmol), THF (1 mL) and H₂O(0.2 mL). The reaction mixture was stirred at 25° C. for 1 hour. The reaction mixture was concentrated under reduced pressure to afford (R)-22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8, 11, 14, 17-hexaoxa-21-azatetracosan-24-oic acid (90 mg, 90 μmol, 100%) as a light yellow solid, which was used directly for next step without any purification.

Step 5: Into an 8-mL vial, was placed a mixture of (R)-22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8,11,14,17-hexaoxa-21-azatetracosan-24-oic acid (90 mg, 1 Eq, 90 μmol), TCFH (30 mg, 1.2 Eq, 0.11 mmol), NMI (22 mg, 3.0 Eq, 0.27 mmol) and DMF (0.7 mL). The reaction mixture was stirred at 20° C. for 10 minutes, then tert-butyl 2,5-diazaspiro[3.4]octane-5-carboxylate (38 mg, 2.0 Eq, 0.18 mmol)_{w} as added and the reaction mixture was stirred at 25° C. for an additional 1 hour. Finally, DBU (27 mg, 27 μL, 2.0 Eq, 0.18 mmol)_{w} as added and the reaction mixture was stirred at 25° C. for an additional 1 hour. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide tert-butyl(R)-2-(22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8, 11, 14, 17-hexaoxa-21-azatetracosan-24-oyl)-2,5-diazaspiro[3.4]octane-5-carboxylate (70 mg, 59 μmol, 65%) as a yellow solid. Calc'd for C₅₉H₇₇C₁₂N₇O₁₅:1195.20, found [M+H]⁺: 1194.4, 1196.4.

Step 6: Into an 8-mL vial, was placed a mixture of tert-butyl(R)-2-(22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8,11,14,17-hexaoxa-21-azatetracosan-24-oyl)-2,5-diazaspiro[3.4]octane-5-carboxylate (60 mg, 1 Eq, 50 μmol) and DCM (3 mL), to which was added TFA (0.2 mL). The reaction mixture was stirred at 25° C. for 1 hour. The mixture was concentrated under reduced pressure to provide (R)—N-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,4-dioxo-4-(2,5-diazaspiro[3.4]octan-2-yl) butan-2-yl)-2,5,8,11, 14,17-hexaoxaicosan-20-amide (55 mg, 50 μmol, 100%) as a white solid, which was used directly for next step without any purification. Calc'd for C₅₄H₆₉C₁₂N₇O₁₃:1093.43, found [M+H]: 1094.4, 1096.4.

Step 7: Into an 8-mL vial, was placed a mixture of (tert-butoxycarbonyl)glycine (16 mg, 1.0 Eq, 91 μmol), HATU (21 mg, 0.60 Eq, 55 μmol), DIEA (18 mg, 24 μL, 1.5 Eq, 0.14 mmol) and DMF (0.9 mL). The reaction mixture was stirred at 20° C. for 10 minutes, then (R)—N-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,4-dioxo-4-(2,5-diazaspiro[3.4]octan-2-yl) butan-2-yl)-2,5,8,11, 14, 17-hexaoxaicosan-20-amide (50 mg, 0.5 Eq, 46 μmol)_{w} as added and the reaction mixture was stirred at 25° C. for an additional 1 hour. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were combined to provide tert-butyl(R)-(2-(2-(22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8, 11, 14, 17-hexaoxa-21-azatetracosan-24-oyl)-2,5-diazaspiro[3.4]octan-5-yl)-2-oxoethyl) carbamate (26 mg, 21 μmol, 23%) as a yellow solid. Calc'd for C₆₁H₈₀C₁₂N₈O₁₆:1250. 51, found [M+H]⁺: 1251.5, 1253.5.

Step 8: Into an 8-mL vial, was placed a mixture of tert-butyl(R)-(2-(2-(22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8,11,14,17-hexaoxa-21-azatetracosan-24-oyl)-2,5-diazaspiro[3.4]octan-5-yl)-2-oxoethyl) carbamate (26 mg, 1 Eq, 21 μmol) and DCM (2 mL), to which was added TFA (0.1 mL). The reaction mixture was stirred at 25° C. for 1 hour. The mixture was concentrated under reduced pressure to provide (R)—N-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-(5-glycyl-2,5-diazaspiro[3.4]octan-2-yl)-1,4-dioxobutan-2-yl)-2,5,8, 11, 14, 17-hexaoxaicosan-20-amide (26 mg, 23 μmol, 110%) as a white solid, which was used directly in the next step without any purification. Calc'd for C₅₆H₇₂C₁₂N&O14:1150.45, found [M+H]: 1151.4, 1153.4.

Step 9: Into an 8-mL round bottom flask, was placed a mixture of (R)—N-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-(5-glycyl-2,5-diazaspiro[3.4]octan-2-yl)-1,4-dioxobutan-2-yl)-2,5,8, 11, 14, 17-hexaoxaicosan-20-amide (26 mg, 1 Eq, 23 μmol), DIEA (8.8 mg, 12 μL, 3.0 Eq, 68 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (23 mg, 2.0 Eq, 46 μmol) and DMF (0.5 mL). The reaction mixture was stirred at 25° C. for 1 hour. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(2-((2-(2-(22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-20-oxo-2,5,8,11,14,17-hexaoxa-21-azatetracosan-24-oyl)-2,5-diazaspiro[3.4]octan-5-yl)-2-oxoethyl)amino)-2-oxoethyl)-1,4,7,10-tetraazacyclododecane-1,4,7-triyl)triacetic acid--2,2,2-trifluoroacetic acid (1/1) (5.8 mg, 3.5 μmol, 16%) as a white solid. Calc'd for C₇₄H₉₉C₁₂F3N₁₂O₂₃:1650.63, found [M+H−TFA]: 1537.9, 1538.9.

### Example 26: (S)-2,2′,2″-(10-(21-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,5,21-trioxo-4-(sulfomethyl)-9,12,15,18-tetraoxa-3,6-diazahenicosyl)-1,4,7,10-tetraazacyclododecane-1,4,7-triyl)triacetic acid (Compound 26)

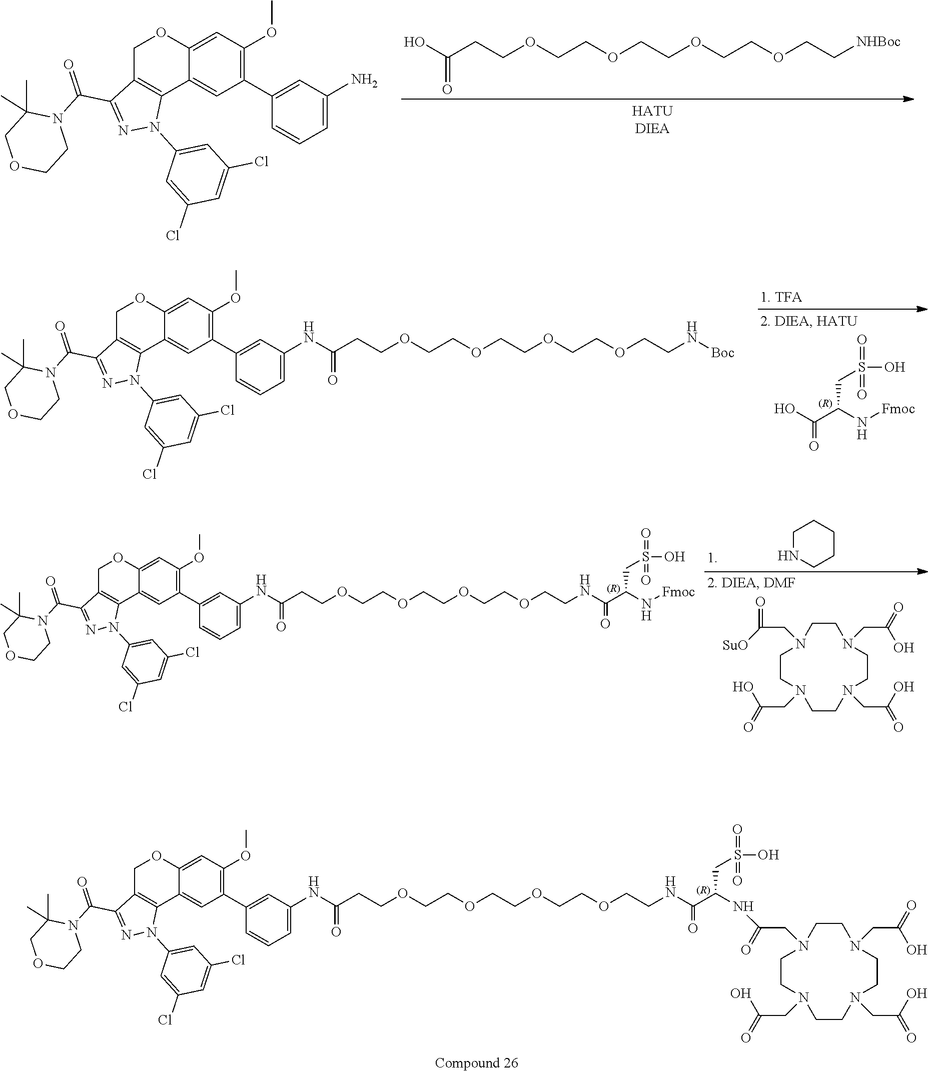

Step 1: Into a 40-mL vial, was placed a mixture of 2,2-dimethyl-4-oxo-3,8, 11,14,17-pentaoxa-5-azaicosan-20-oic acid (64 mg, 1.0 Eq, 0.18 mmol), HATU (79 mg, 1.2 Eq, 0.21 mmol), DIEA (68 mg, 92 μL, 3.0 Eq, 0.53 mmol) and DMF (1.0 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1 Eq, 173 μmol)_{w} as added and the reaction mixture was stirred at 25° C. for an additional 2 hours. The mixture was directly purified by MPLC with the following conditions: Column, WelFlash™, C18 120 g, Spherical 20-40 μm; Mobile phase, Water (0.05% FA) and ACN (5% ACN to 5%

ACN in 1 min, 30% ACN up to 98% in 6 min, 98% ACN to 98% in 3 min); Total flow rate, 70 mL/min; Detector, UV 220 nm. Purification provided tert-butyl(15-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-15-oxo-3,6,9,12-tetraoxapentadecyl) carbamate (105 mg, 113 μmol, 65.6%) as a yellow oil. Calc'd for C₄₆H₅₇C₁₂N₅O₁₁:925.34, found [M+H]: 926.2.

Step 2: Into an 8-mL vial, was placed a mixture of tert-butyl(15-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-15-oxo-3,6,9,12-tetraoxapentadecyl) carbamate (105 mg, 1 Eq, 113 μmol) and DCM (3.0 mL), to which was added TFA (0.15 mL). The reaction mixture was stirred at 25° C. for 1 hour. The mixture was concentrated under reduced pressure to provide 1-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,6,9,12-tetraoxapentadecan-15-amide (82 mg, 99 μmol, 88%) as a white solid. Calc'd for C₄₁H₄₉C₁₂N₅O₉:825.29, found [M+H]⁺: 826.3. Step 3: Into a 40-mL vial, was placed a mixture of (((9H-fluoren-9-yl) methoxy) carbonyl) (sulfo)-D-alanine (32 mg, 1.0 Eq, 82 μmol), DIEA (75 mg, 0.10 mL, 7.4 Eq, 0.58 mmol), HATU (38 mg, 1.3 Eq, 0.10 mmol) and DMF (1.0 mL). The reaction mixture was stirred at 20° C. for 10 minutes, then 1-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,6,9,12-tetraoxapentadecan-15-amide was added and the reaction mixture was stirred at 25° C. for an additional 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided (R)-2-((((9H-fluoren-9-yl) methoxy) carbonyl)amino)-19-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3, 19-dioxo-7, 10,13, 16-tetraoxa-4-azanonadecane-1-sulfonic acid (70 mg, 58 μmol, 74%) as a yellow oil. Calc'd for C₅₉H₆₄C₁₂N₆₀₁₅S: 1198.35, found [M+H]: 1199.5.

Step 4: Into an 8-mL vial, was placed a mixture of (R)-2-((((9H-fluoren-9-yl) methoxy) carbonyl)amino)-19-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,19-dioxo-7,10, 13, 16-tetraoxa-4-azanonadecane-1-sulfonic acid, piperidine (50 mg, 58 μL, 11 Eq, 0.59 mmol) and DMF (1.0 mL). The reaction mixture was stirred at 25° C. for 2 hours. The mixture was concentrated under reduced pressure. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide (R)-2-amino-19-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3, 19-dioxo-7, 10, 13, 16-tetraoxa-4-azanonadecane-1-sulfonic acid (50 mg, 51 μmol, 94%) as a white solid. Calc'd for C₄H₅₄C₁₂N₆₀₁₃S: 976.28, found [M+H]⁺: 977.3.

Step 5: Into a 40-mL vial, was placed a mixture of (R)-2-amino-19-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,19-dioxo-7, 10,13, 16-tetraoxa-4-azanonadecane-1-sulfonic acid (45 mg, 1 Eq, 46 μmol), DIEA (38 mg, 51 μL, 6.4 Eq, 0.29 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (48 mg, 2.1 Eq, 96 μmol) and DMF (1 mL). The reaction mixture was stirred at 25° C. for an additional 2 hours. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(21-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,5,21-trioxo-4-(sulfomethyl)-9,12,15, 18-tetraoxa-3,6-diazahenicosyl)-1,4,7,10-tetraazacyclododecane-1,4,7-triyl)triacetic acid (23.2 mg, 17.0 mmol, 37%) as a yellow oil. Calc'd for C₆₀H₈₀C₁₂N₁₀₀₂₀S: 1362.47, found [M+H]]: 1363.60.

### Example 27: (R)-2,2′,2″-(10-(4-(2-carboxyethyl)-21-((3-(1-(3,5-dichlorophenyl)-3-(3,3-dimethylmorpholine-4-carbonyl)-7-methoxy-1,4- dihydrochromeno|4,3-clpyrazol-8-yl)phenyl)amino)-2,5,21-trioxo-9,12,15,18-tetraoxa-3,6-diazahenicosyl)-1,4,7,10-tetraazacyclododecane-1,4,7-triyl)triacetic acid (Compound 27)

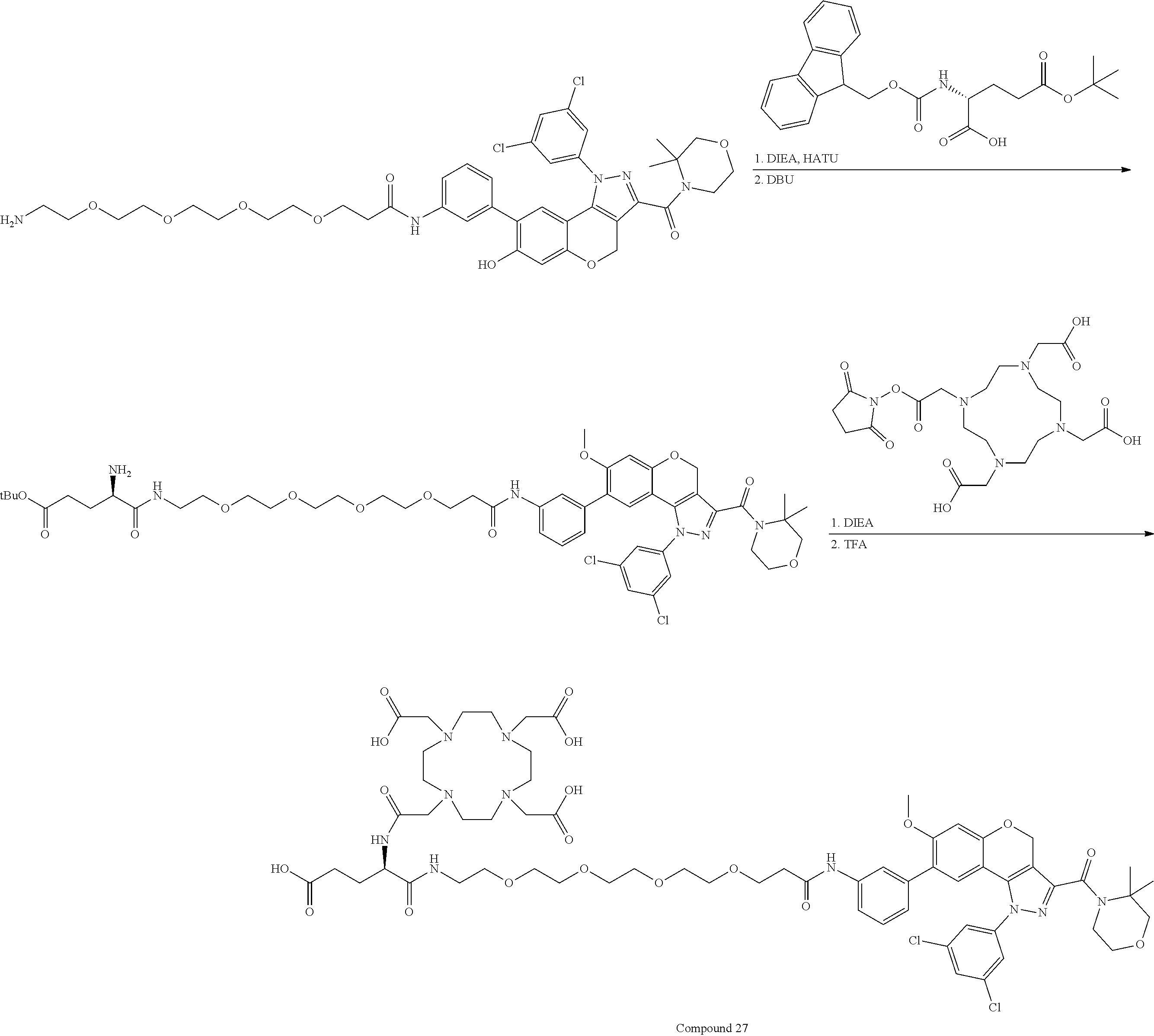

Step 1: Into a 40-mL vial, was placed a mixture of (R)-2-((((9H-fluoren-9-yl) methoxy) carbonyl)amino)-5-(tert-butoxy)-5-oxopentanoic acid (25 mg, 1.4 Eq, 59 μmol)₂-(3H-[1,2,3]triazolo[4,5-b]pyridin-3-yl)-1,1,3,3-tetramethylisouronium hexafluorophosphate (V) (from Example 21, Step 2; 30 mg, 1.8 Eq, 79 μmol), N-ethyl-N-isopropylpropan-2-amine (25 mg, 4.5 Eq, 0.19 mmol) and DMF (1 mL). The reaction mixture was stirred at 20° C. for 10 minutes, then 1-amino-N-(3-(1-(3,5-dichlorophenyl)-3-(3,3-dimethylmorpholine-4-carbonyl)-7-hydroxy-1,4-dihydrochromeno[4,3-c]pyrazol-8-yl)phenyl)-3,6,9,12-tetraoxapentadecan-15-amide (35 mg, 1 Eq, 43 μmol), was added and the reaction mixture was stirred at 25° C. for an additional 1 hour. The crude product tert-butyl(R)-18-((((9H-fluoren-9-yl) methoxy) carbonyl)amino)-1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,17-dioxo-4,7,10, 13-tetraoxa-16-azahenicosan-21-oate was used directly in the next step without any purification. Calc'd for C₆₅H₇₄C₁₂N₆O₁₄:1232.46, found [M+H]⁺: 1233.4, 1235.4.

Step 2: Into the 8-mL vial was place a mixture of tert-butyl(R)-18-((((9H-fluoren-9-yl) methoxy) carbonyl)amino)-1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,17-dioxo-4,7,10, 13-tetraoxa-16-azahenicosan-21-oate and DBU (10 mg, 9.9 μL, 2.3 Eq, 66 μmol). The reaction mixture was stirred at 25° C. for 1 hour. The mixture was concentrated under reduced pressure. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide tert-butyl(R)-18-amino-1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,17-dioxo-4,7, 10, 13-tetraoxa-16-azahenicosan-21-oate (11 mg, 11 μmol, 38%) as a white solid. Calc'd for C₅₀H₆₄C₁₂N₆O₁₂:1010. 39, found [M+H]⁺: 1011.3,1013.3.

Step 3: Into a 2-mL vial, was placed a mixture of tert-butyl(R)-18-amino-1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,17-dioxo-4,7, 10, 13-tetraoxa-16-azahenicosan-21-oate (10 mg, 1 Eq, 9.9 μmol), DIEA (5 mg, 7 μL, 4 Eq, 0.04 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (11 mg, 2.2 Eq, 22 μmol) and DMF (1 mL). The reaction mixture was stirred at 25° C. for 1 hour. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization. Calc'd for C₆₆H₉₀C₁₂N₁₀O₁₉:1396.58, found [M+H]: 1397.4, 1399.4.

Step 4: Into an 8-mL vial, was placed a mixture of (R)-2,2′,2″-(10-(4-(3-(tert-butoxy)-3-oxopropyl)-21-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,5,21-trioxo-9, 12, 15, 18-tetraoxa-3,6-diazahenicosyl)-1,4,7,10-tetraazacyclododecane-1,4,7-triyl)triacetic acid (10 mg, 1 Eq, 7.2 μmol) and DCM (0.15 mL), to which was added TFA (0.01 mL). The reaction mixture was stirred at 25° C. for 1 hour. The mixture was concentrated under reduced pressure. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(4-(2-carboxyethyl)-21-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,5,21-trioxo-9, 12,15,18-tetraoxa-3,6-diazahenicosyl)-1,4,7,10-tetraazacyclododecane-1,4,7-triyl)triacetic acid--2,2,2-trifluoroacetic acid (1/1) (3.3 mg, 2.3 μmol, 32%) as a white solid. Calc'd for C₆₄H₈₃C₁₂F3N₁₀O₂₁:1454.51, found [M+H−TFA]⁺: 1341.6, 1343.6.

### Example 28:2,2′,2″-(10-(20-carboxy-1-((3-(1-(3,5-dichlorophenyl)-3-(3,3-dimethylmorpholine-4-carbonyl)-7-methoxy-1,4-dihydrochromeno|4,3- c]pyrazol-8-yl)phenyl)amino)-1,17-dioxo-4,7,10,13-tetraoxa-16-azaicosan-20-yl)-1,4,7,10-tetraazacyclododecane-1,4,7-triyl)triacetic acid (Compound 28)

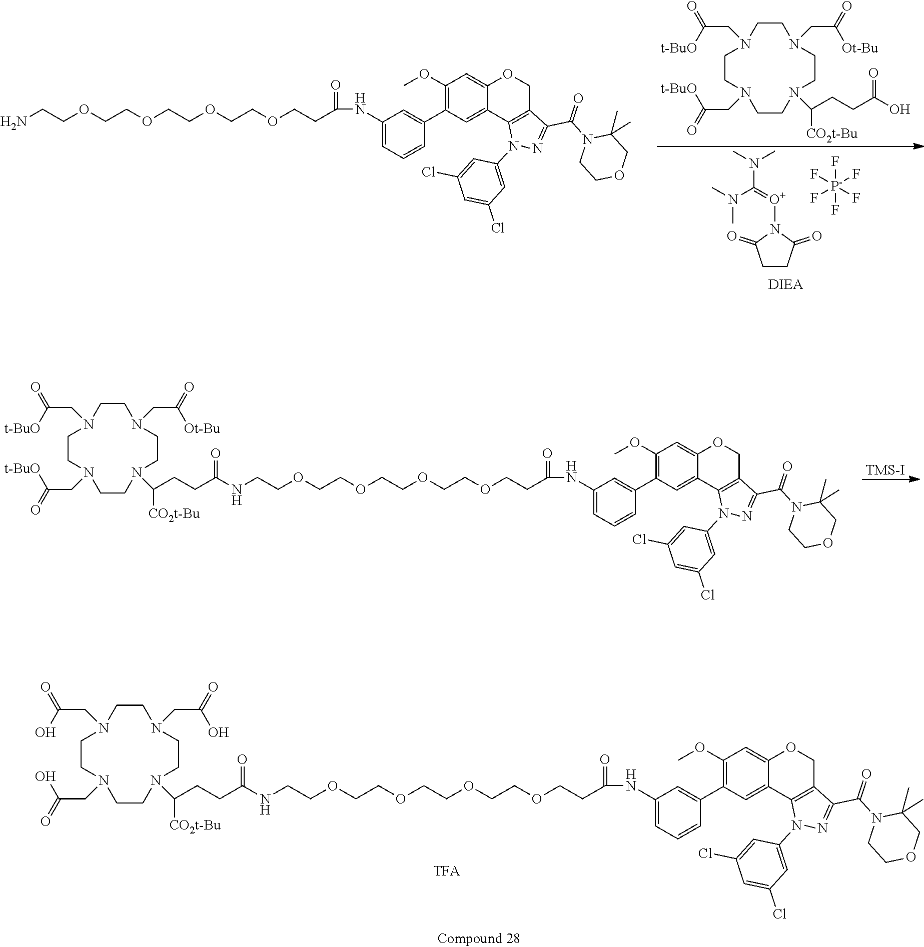

Step 1: Into a 40-mL vial, was placed a mixture of 5-(tert-butoxy)-5-oxo-4-(4,7,10-tris(2-(tert-butoxy)-2-oxoethyl)-1,4,7,10-tetraazacyclododecan-1-yl) pentanoic acid (from Example 29, Step 2; 100 mg, 1.18 Eq, 143 μmol), 2-(2,5-dioxopyrrolidin-1-yl)-1,1,3,3-tetramethyluronium hexafluorophosphate (V) (62 mg, 1.4 Eq, 0.17 mmol), DIEA (70 mg, 94 μL, 4.5 Eq, 0.54 mmol) and DMF (1 mL). The reaction mixture was stirred at 20° C. for 10 minutes, then 5-(tert-butoxy)-5-oxo-4-(4,7,10-tris(2-(tert-butoxy)-2-oxoethyl)-1,4,7,10-tetraazacyclododecan-1-yl) pentanoic acid (100 mg, 1.18 Eq, 143 μmol), was added and the reaction mixture was stirred at 25° C. for an additional 1 hour. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide tri-tert-butyl 2,2′,2″-(10-(24-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,2-dimethyl-4,8,24-trioxo-3,12, 15, 18,21-pentaoxa-9-azatetracosan-5-yl)-1,4,7,10-tetraazacyclododecane-1,4,7-triyl)triacetate (70 mg, 46 μmol, 38%) as a white solid. Calc'd for C₇₆HinC12N₉O₁₈:1507.74, found [M+H]⁺: 1508.9, 1510.9.

Step 2: Into an 8-mL vial, was placed a mixture of tri-tert-butyl 2,2′,2″-(10-(24-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,2-dimethyl-4,8,24-trioxo-3, 12,15, 18,21-pentaoxa-9-azatetracosan-5-yl)-1,4,7,10-tetraazacyclododecane-1,4,7-triyl)triacetate (70 mg, 1 Eq, 46 μmol) and DCM (1 mL), to which was added DCM (1 mL). The reaction mixture was stirred at 25° C. for 1 hour. The mixture was concentrated under reduced pressure then 2 mL water was added followed by IN NaOH to adjust the solution to pH=7. Additional DMF (3 ml)_{w} as added. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(22-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,6,22-trioxo-2, 10, 13, 16, 19-pentaoxa-7-azadocosan-3-yl)-1,4, 7, 10-tetraazacyclododecane-1,4,7-triyl)triacetic acid--2,2,2-trifluoroacetic acid (1/1) (49.5 mg, 35.4 μmol, 76%) as a white solid. Calc'd for C₆₀H₇₉C₁₂N₉O₁₈:1283.49, found [M+H]⁺: 1284.6, 1286.6.

### Example 29:2,2′,2″-(10-(2-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-2-oxoethyl)-1,4,7,10-tetraazacyclododecane-1,4,7-triyl)triacetic acid (Compound 29)

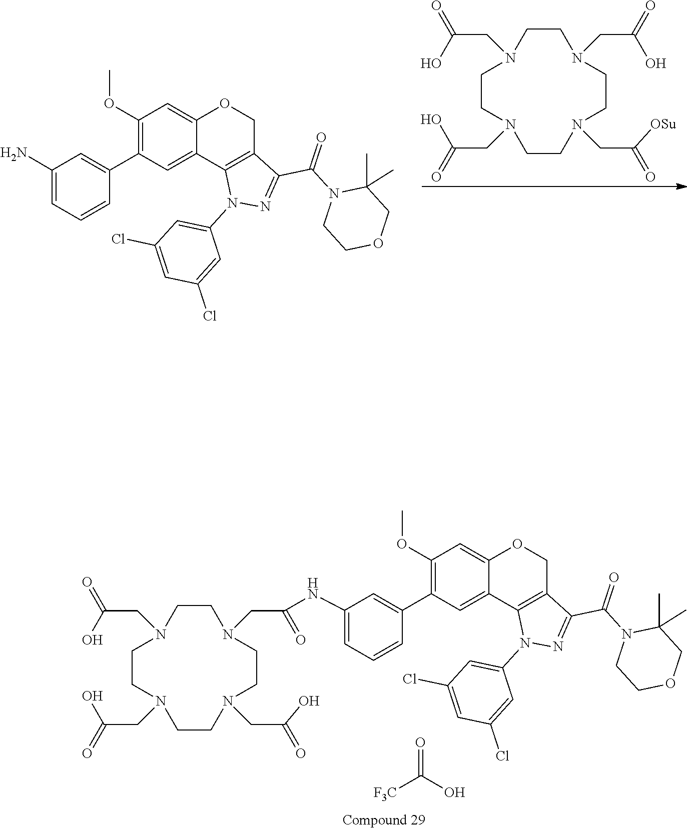

Step 1: Into an 8-mL vial was placed (8-(3-aminophenyl)-1-(3,5-dichlorophenyl)-7-methoxy-1,4-dihydrochromeno[4,3-c]pyrazol-3-yl) (3,3-dimethylmorpholino) methanone (50 mg, 1 Eq, 86 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (87 mg, 2.0 Eq, 0.17 mmol) and pyridine (0.5 mL). The mixture was stirred for 2 hours at 120° C. The reaction mixture was purified by MPLC using the following conditions: Column, C18 120 g; mobile phase, Water (0.05% TFA) and ACN (12% ACN up to 50% in 6 min); Total flow rate, 70 mL/min; Detector, UV 220 nm. The collected fractions were concentrated under reduced pressure and dried to provide the title compound as an off-white solid. Calc'd for C₄₈H₅₅Cl2F3N₈O₁₃:1078.32, found [M+H−TFA]⁺: 965.5.

### Example 30: (R)-2,2′,2″-(10-(2-((1-carboxy-3-((3-(1-(3,5-dichlorophenyl)-3-(3,3-dimethylmorpholine-4-carbonyl)-7-methoxy-1,4- dihydrochromeno|4,3-c]pyrazol-8-yl)phenyl)amino)-3-oxopropyl)amino)-2-oxoethyl)-1,4,7,10-tetraazacyclododecane-1,4,7-triyl)triacetic acid (Compound 30)

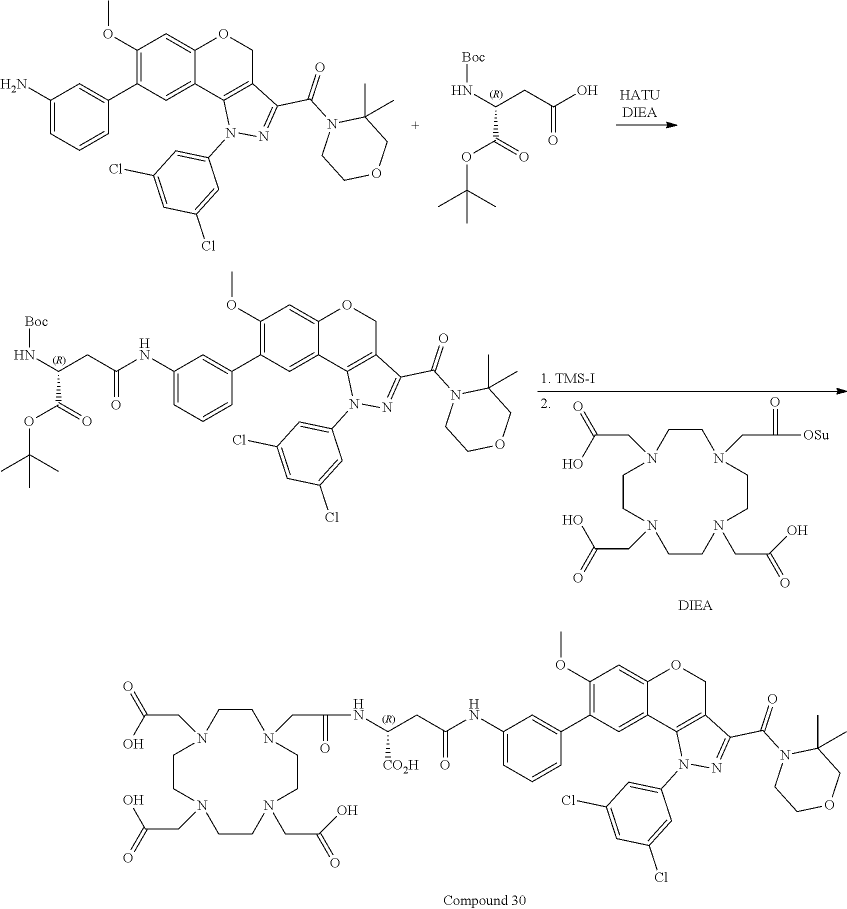

Step 1: Into a 8-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (150 mg, 1 Eq, 259 μmol), (R)-4-(tert-butoxy)-3-((tert-butoxycarbonyl)amino)-4-oxobutanoic acid (115 mg, 1.54 Eq, 397 μmol), DIEA (100 mg, 135 μL, 2.99 Eq, 774 μmol) and DMF (2 mL). The reaction mixture was stirred at 20° C. for 10 minutes, then HATU (120 mg, 1.22 Eq, 316 μmol)_{w} as added and the reaction mixture was stirred at 25° C. for an additional 1 hour. The mixture was diluted with water (50 mL), extracted with EtOAc (50 mL×3), then the combined organic layers were washed with water (50 mL×2) and brine (50 mL), dried over anhydrous Na₂SO₄ and concentrated under reduced pressure. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 95% B in 8 min; Flow rate: 20 mL/min; Wave Length: 220 nm. The collected fractions were dried by lyophilization to provide tert-butyl N₂-(tert-butoxycarbonyl)-N₄-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-D-asparaginate (220 mg, 249 μmol, 96.3%, 96.4% Purity) as an off-white solid. Calc'd for C₄₃H₄₉C₁₂N₅O₉:849.29, found [M+H]⁺: 850.1.

Step 2: Into a 8-mL vial, was placed a mixture of tert-butyl N₂-(tert-butoxycarbonyl)-N₄-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-D-asparaginate (200 mg, 1 Eq, 235 μmol) and TMS-I (150 mg, 102 μL, 3.19 Eq, 750 μmol), to which was added DCM (2 mL). The reaction mixture was stirred at 25° C. for 2 hours. The mixture was concentrated under reduced pressure. The crude product was purified by Prep-HPLC with the following conditions:

Column: SunFire prep OBD 19*150 mm 5 μm; Mobile Phase A: Water (0.05% FA); Mobile Phase B: ACN; Gradient: 25% B to 98% B in 10 min; Flow rate: 20 mL/min; Wave Length: 220 nm. The collected fractions were dried by lyophilization to provide N₄-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-D-asparagine (120 mg, 0.16 mmol, 66%, 90% Purity) as a white solid. Calc'd for C₃₄H₃₃Cl2N₅O₇:693.18, found [M+H]: 693.7.

Step 3: Into a 8-mL vial, was placed a mixture of N₄-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-D-asparagine (110 mg, 1 Eq, 158 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (240 mg, 3.02 Eq, 479 μmol), DIEA (180 mg, 243 μL, 8.79 Eq, 1.39 mmol) and DMF (1.5 mL). The reaction mixture was stirred at 25° C. for an additional 1 hour. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(2-((1-carboxy-3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-2-oxoethyl)-1,4,7,10-tetraazacyclododecane-1,4,7-triyl)triacetic acid (49.4 mg, 44.4 μmol, 28.0%, 97.2% Purity) as a white solid. Calc'd for C₅₀H₅₉C₁₂N₉O₁₄:1079.36, found [M+H]⁺: 1080.5.

### Example 31: (R)-2,2′,2″-(10-(2-((1-carboxy-4-((3-(1-(3,5-dichlorophenyl)-3-(3,3-dimethylmorpholine-4-carbonyl)-7-methoxy-1,4- dihydrochromeno|4,3-c]pyrazol-8-yl)phenyl)amino)-4-oxobutyl)amino)-2-oxoethyl)-1,4,7,10-tetraazacyclododecane-1,4,7-triyl)triacetic acid (Compound 31)

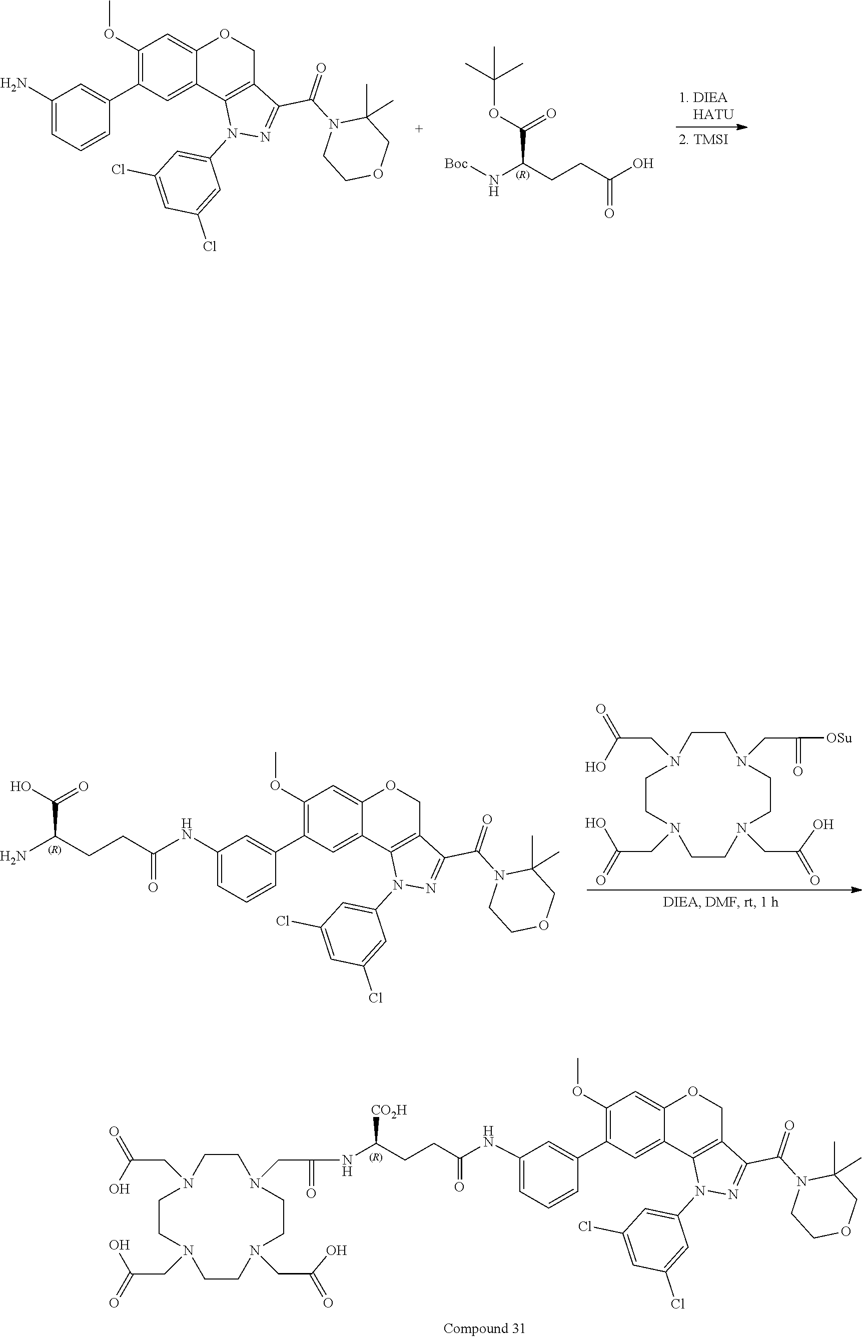

Step 1: Into a 8-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (130 mg, 1 Eq, 224 μmol), (R)-5-(tert-butoxy)-4-((tert-butoxycarbonyl)amino)-5-oxopentanoic acid (85 mg, 1.2 Eq, 0.28 mmol), DIEA (100 mg, 135 μL, 3.45 Eq, 774 μmol) and DMF (1.5 mL). The reaction mixture was stirred at 20° C. for 10 minutes, then HATU (110 mg, 1.29 Eq, 289 μmol)_{w} as added and the reaction mixture was stirred at 25° C. for an additional 1 hour. The mixture was purified by Prep-HPLC using the following conditions: Column: SunFire prep OBD 19*150 mm 5 μm; Mobile Phase A: Water (0.05% FA); Mobile Phase B: ACN; Gradient: 25% B to 95% B in 8 min; Flow rate: 60 mL/min; Wave Length: 220 nm. The collected fractions were dried by lyophilization to provide tert-butyl N₂-(tert-butoxycarbonyl)-N₅-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-D-glutaminate (200 mg, 0.21 mmol, 93%, 90% Purity) as an off-white solid. Calc'd for C₄H₅₁C₁₂N₅O₉:863.31, found [M+H]⁺: 864.3.

Step 2: Into an 8-mL vial, was placed a mixture of tert-butyl N₂-(tert-butoxycarbonyl)-N₅-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-D-glutaminate (200 mg, 1 Eq, 231 μmol) and TMS-I (140 mg, 95.2 μL, 3.03 Eq, 700 μmol), to which was added DCM (2 mL). The reaction mixture was stirred at 25° C. for 1 hour. The mixture was concentrated under reduced pressure. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 98% B in 10 min; Flow rate: 20 mL/min; Wave Length: 220 nm. The collected fractions were dried by lyophilization to provide N₅-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-D-glutamine (110 mg, 111 mmol, 48.1%, 71.6% Purity) as a white solid. Calc'd for C₃₅H₃₅Cl2N₅O₇:707.19, found [M+H]⁺: 708.1.

Step 3: Into an 8-mL vial, was placed a mixture of N₅-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-D-glutamine (110 mg, 1 Eq, 155 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (160 mg, 2.06 Eq, 319 μmol), DIEA (70 mg, 94 μL, 3.5 Eq, 0.54 mmol) and DMF (1.5 mL). The reaction mixture was stirred at 25° C. for 1 hour. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(2-((1-carboxy-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutyl)amino)-2-oxoethyl)-1,4,7,10-tetraazacyclododecane-1,4,7-triyl)triacetic acid (55.8 mg, 49.4 μmol, 31.8%, 96.9% Purity) as a white solid. Calc'd for C₅₁H₆₁C₁₂N₉O₁₄:1093.37, found [M+H]⁺: 1094.5.

### Example 32: (R)-2,2′,2″-(10-(2-((1-carboxy-3-((2-((3-(1-(3,5-dichlorophenyl)-3-(3,3-dimethylmorpholine-4-carbonyl)-7-methoxy-1,4- dihydrochromeno|4,3-clpyrazol-8-yl)phenyl)amino)-2-oxoethyl)amino)-3-oxopropyl)amino)-2-oxoethyl)-1,4,7,10-tetraazacyclododecane-1,4,7-triyl)triacetic acid (Compound 32)

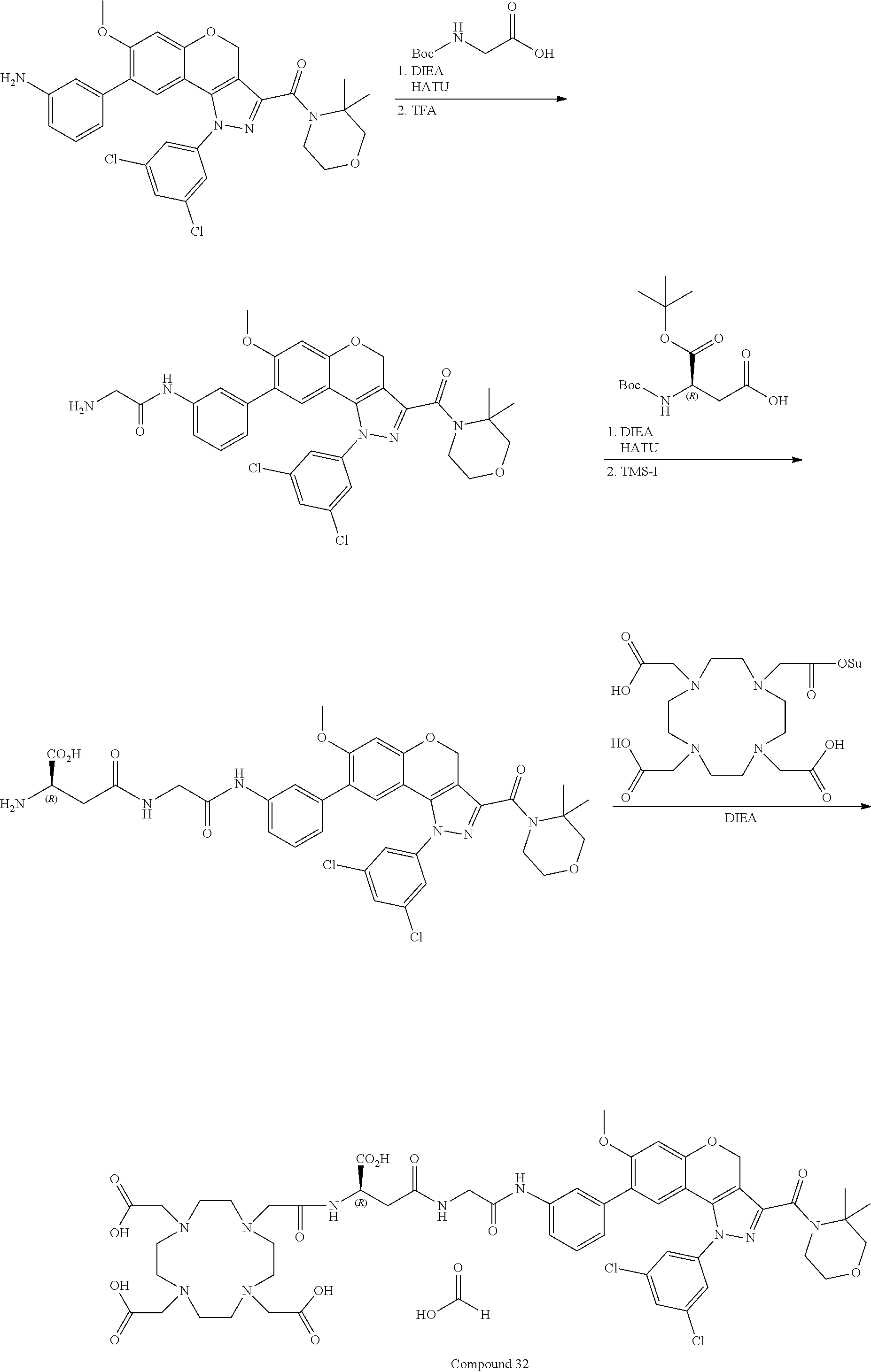

Step 1: Into an 8-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (250 mg, 1 Eq, 431 μmol), (tert-butoxycarbonyl)glycine (90 mg, 1.2 Eq, 0.51 mmol), DIEA (170 mg, 229 μL, 3.05 Eq, 1.32 mmol) and DMF (2.5 mL). The reaction mixture was stirred at 20° C. for 10 minutes, then HATU (200 mg, 1.22 Eq, 526 μmol)_{w} as added and the reaction mixture was stirred at 25° C. for an additional 1 hour. The mixture was purified by Prep-HPLC using the following conditions: Column: SunFire prep OBD 19*150 mm 5 μm; Mobile Phase A: Water (0.05% FA); Mobile Phase B: ACN; Gradient: 25% B to 95% B in 8 min; Flow rate: 60 mL/min; Wave Length: 220 nm. The collected fractions were dried by lyophilization to provide tert-butyl(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) carbamate (320 mg, 0.26 mmol, 60%, 60% Purity) as an off-white solid. Calc'd for C₃₇H₃₉C₁₂N₅O₇:735.22, found [M+H]: 736.1.

Step 2: Into an 8-mL vial, was placed a mixture of tert-butyl(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) carbamate (320 mg, 1 Eq, 434 μmol) and TFA (0.2 mL), to which was added DCM (3.2 mL). The reaction mixture was stirred at 25° C. for 1 hour. The mixture was concentrated under reduced pressure. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 98% B in 10 min; Flow rate: 20 mL/min; Wave Length: 220 nm. The collected fractions were dried by lyophilization to provide 2-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (280 mg, 0.35 mmol, 81%, 80% Purity) as a white solid. Calc'd for C₃₂H₃₁C₁₂N₅O₅:635.17, found [M+H]: 636.1. Step 3: Into an 8-mL vial, was placed a mixture of 2-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (280 mg, 1 Eq, 440 μmol), (R)-4-(tert-butoxy)-3-((tert-butoxycarbonyl)amino)-4-oxobutanoic acid (180 mg, 1.41 Eq, 622 μmol), DIEA (175 mg, 236 μL, 3.08 Eq, 1.35 mmol) and DMF (3 mL). The reaction mixture was stirred at 20° C. for 10 minutes, then HATU (200 mg, 1.20 Eq, 526 μmol)_{w} as added and the reaction mixture was stirred at 25° C. for an additional 1 hour. The mixture was purified by Prep-HPLC using the following conditions: Column: SunFire prep OBD 19*150 mm 5 μm; Mobile Phase A: Water (0.05% FA); Mobile Phase B: ACN; Gradient: 25% B to 95% B in 8 min; Flow rate: 60 mL/min; Wave Length: 220 nm. The collected fractions were dried by lyophilization to provide tert-butyl N₂-(tert-butoxycarbonyl)-N₄-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl)-D-asparaginate (110 mg, 0.11 mmol, 25%, 90% Purity) as an off-white solid. Calc'd for C₄₅H₅₂C₁₂N₆O₁₀:906.31, found [M+H]: 907.2.

Step 4: Into an 8-mL vial, was placed a mixture of tert-butyl N₂-(tert-butoxycarbonyl)-N₄-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl)-D-asparaginate (105 mg, 1 Eq, 116 μmol) and TMS-I (70 mg, 48 μL, 3.0 Eq, 0.35 mmol), to which was added DCM (1 mL). The reaction mixture was stirred at 25° C. for 1 hour. The mixture was concentrated under reduced pressure. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 98% B in 10 min; Flow rate: 20 mL/min; Wave Length: 220 nm. The collected fractions were dried by lyophilization to provide N₄-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl)-D-asparagine (65 mg, 78 μmol, 67%, 90% Purity) as a white solid. Calc'd for C₃₆H₃₆C₁₂N₆O₈:750.20, found [M+H]: 751.4.

Step 5: Into an 8-mL vial, was placed a mixture of N₄-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl)-D-asparagine (65 mg, 1 Eq, 86 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (90 mg, 2.1 Eq, 0.18 mmol), DIEA (60 mg, 81 μL, 5.4 Eq, 0.46 mmol) and DMF (0.7 mL). The reaction mixture was stirred at 25° C. for 2 hours. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(2-((1-carboxy-3-((2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl)amino)-3-oxopropyl)amino)-2-oxoethyl)-1,4,7,10-tetraazacyclododecane-1,4,7-triyl)triacetic acid (38.8 mg, 32.0 μmol, 37%, 93.9% Purity) as a white solid. Calc'd for C₅₂H₆₂C₁₂N₁₀O₁₅:1136.37, found [M+H]⁺: 1137.6.

### Example 33: (R)-2,2′,2″-(10-(2-((1-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-1-oxo-3-sulfopropan-2-yl)amino)-2-oxoethyl)-1,4,7,10-tetraazacyclododecane-1,4,7-triyl)triacetic acid (Compound 33)

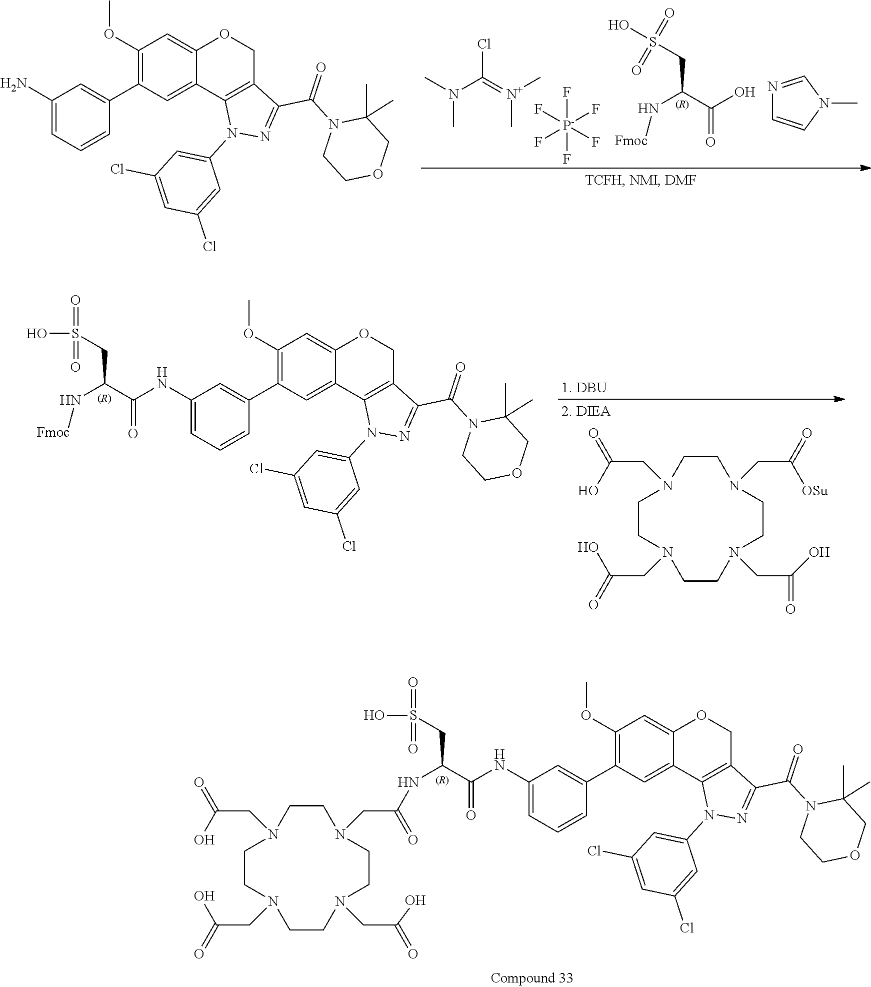

Step 1: Into a 40-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (150 mg, 1 Eq, 259 μmol), (((9H-fluoren-9-yl) methoxy) carbonyl) (sulfo)-D-alanine (304 mg, 3.00 Eq, 777 μmol), chloro-N,N,N′,N′-tetramethylformamidinium hexafluorophosphate (225 mg, 3.1 Eq, 802 μmol), 1-methyl-1H-imidazole (106 mg, 5.0 Eq, 1.29 mmol) and DMF (1.5 mL). The reaction mixture was stirred at 25° C. for 2 hours. The mixture was directly purified by MPLC using the following conditions: Column, WelFlash™, C18 120 g, Spherical 20-40 μm; Mobile phase, Water (0.05% NH₃ H₂O) and ACN (5% ACN to 5% ACN in 1 min, 30% ACN up to 98% in 6 min, 98% ACN to 98% in 3 min); Total flow rate, 70 mL/min; Detector, UV 220 nm. Purification provided (R)-2-((((9H-fluoren-9-yl) methoxy) carbonyl)amino)-3-((3-(1-(3,5-dichlorophenyl)-3-(3,3-dimethylmorpholine-4-carbonyl)-7-methoxy-1,4-dihydrochromeno[4,3-c]pyrazol-8-y1)phenyl)amino)-3-oxopropane-1-sulfonic acid (105 mg, 110 μmol, 42.6%) as a yellow oil. Calc'd for C₄₈H₄₃C₁₂N₅O₁₀S: 951.21, found [M+H]⁺: 952.1.

Step 2: Into an 8-mL vial, was placed a mixture of (R)-2-((((9H-fluoren-9-yl) methoxy) carbonyl)amino)-3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropane-1-sulfonic acid (105 mg, 1 Eq, 110 μmol) and DBU (98 mg, 97 μL, 5.8 Eq, 0.64 mmol), to which was added DMF (1 mL). The reaction mixture was stirred at 25° C. for 1 hour. The mixture was concentrated under reduced pressure to provide (R)-2-amino-3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropane-1-sulfonic acid (45 mg, 62 μmol, 56%) as a white solid. Calc'd for C₃₃H₃₃Cl2N₅O&S: 729.14, found [M+H]: 730.2.

Step 3: Into an 8-mL vial, was placed a mixture of (R)-2-amino-3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropane-1-sulfonic acid (45 mg, 1 Eq, 62 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (93 mg, 3.0 Eq, 0.19 mmol), DIEA (47 mg, 63 μL, 5.9 Eq, 0.36 mmol) and DMF (0.45 mL). The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(2-((1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1-oxo-3-sulfopropan-2-yl)amino)-2-oxoethyl)-1,4,7,10-tetraazacyclododecane-1,4,7-triyl)triacetic acid (23.5 mg, 21.0 μmol, 34%) as a yellow oil. Calc'd for C₄₉H₅₉C₁₂N₉O₁₅S: 1115.32, found [M+H]⁺: 1116.5.

### Example 34:2,2′,2″-(10-(2-((((2S,3R,4R,5R,6S)-6-((3-(1-(3,5-dichlorophenyl)-3-(3,3dimethylmorpholine-4-carbonyl)-7-methoxy-1,4-dihydrochromeno|4,3-c]pyrazol-8-yl)phenyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid (Compound 34)

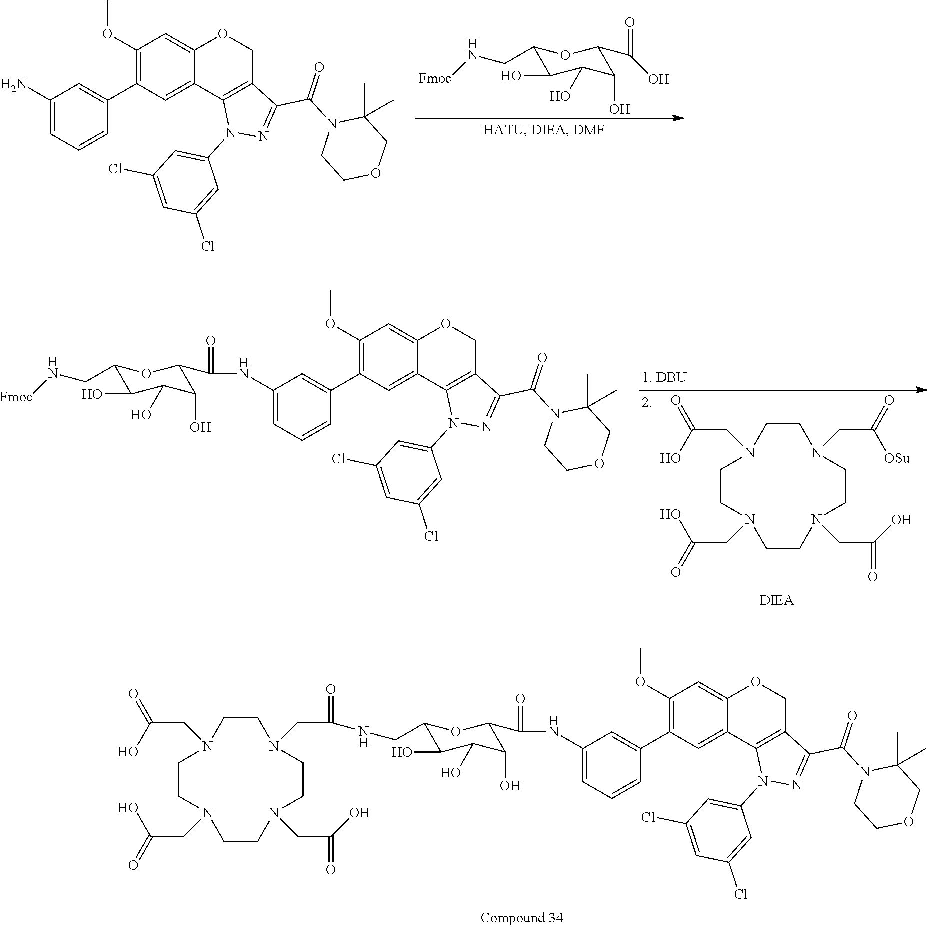

Step 1: Into a 40-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (150 mg, 1 Eq, 259 μmol), (2S,3R,4R,5R,6S)-6-(((((9H-fluoren-9-yl) methoxy) carbonyl)amino)methyl)-3,4,5-trihydroxytetrahydro-2H-pyran-2-carboxylic acid (145 mg, 1.30 Eq, 338 μmol), HATU (149 mg, 1.51 Eq, 392 μmol), DIEA (100 mg, 135 μL, 2.99 Eq, 774 μmol) and DMF (1.5 mL). The reaction mixture was stirred at 25° C. for 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided (9H-fluoren-9-yl)methyl (((2S,3R,4R,5R,6S)-6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-3,4,5-trihydroxytetrahydro-2H-pyran-2-yl)methyl) carbamate (122 mg, 123 μmol, 47.6%) as a yellow oil. Calc'd for C₅₂H₄₉C₁₂N₅O₁₁:989.28, found [M+H]⁺: 990.2.

Step 2: Into an 8-mL vial, was placed a mixture of (9H-fluoren-9-yl)methyl (((2S,3R,4R,5R,6S)-6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-3,4,5-trihydroxytetrahydro-2H-pyran-2-yl)methyl) carbamate (117 mg, 1 Eq, 118 μmol) and DBU (108 mg, 107 μL, 6.01 Eq, 709 μmol), to which was added DMF (1.2 mL). The reaction mixture was stirred at 25° C. for 1 hour. The mixture was concentrated under reduced pressure to provide (2S,3R,4R,5R,6S)-6-(aminomethyl)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,4,5-trihydroxytetrahydro-2H-pyran-2-carboxamide (130 mg, 73 μmol, 62%, 43% Purity) as a white solid. Calc'd for C₃₇H₃₉C₁₂N₅O₉:767.21, found [M+H]⁺: 768.2.

Step 3: Into a 40-mL vial, was placed a mixture of (2S,3R,4R,5R,6S)-6-(aminomethyl)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,4,5-trihydroxytetrahydro-2H-pyran-2-carboxamide (130 mg, 43% Wt, 1 Eq, 72.7 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (73 mg, 2.0 Eq, 0.15 mmol), DIEA (38 mg, 51 μL, 4.0 Eq, 0.29 mmol) and DMF (1.3 mL). The reaction mixture was stirred at 25° C. for 2 hours. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-((((2S,3R,4R,5R,6S)-6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid (42.8 mg, 37.1 μmol, 51.0%) as a white solid. Calc'd for C₅₃H₆₅C₁₂N₉O16:1153.39, found [M+H]⁺: 1154.6.

### Example 35: (Compound 35)

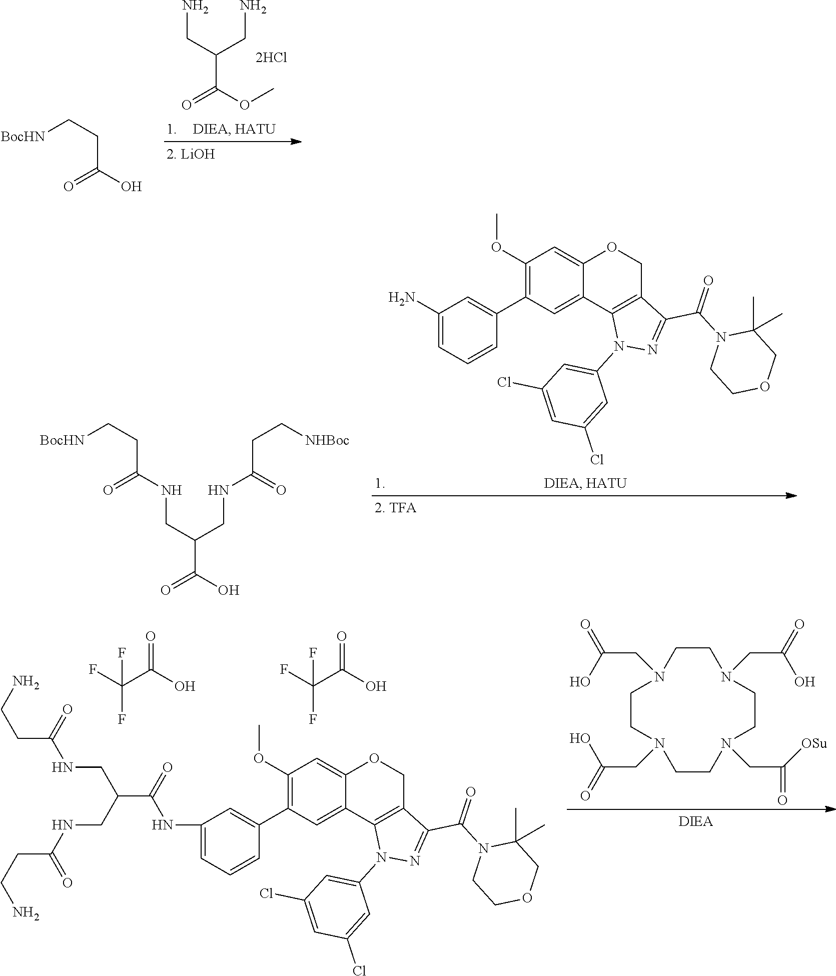

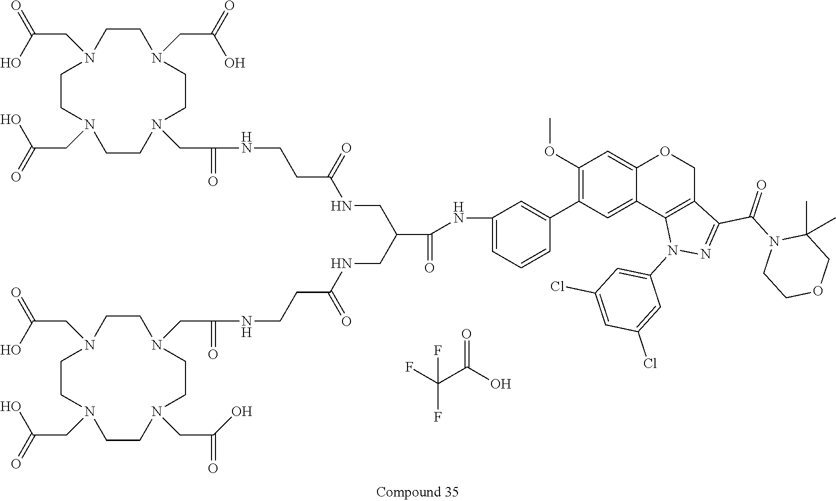 [SMILES: COc1cc2c(cc1-c1cccc(NC(=O)C(CNC(=O)CCNC(=O)CN3CCN(CC(=O)O)CCN(CC(=O)O)CCN(CC(=O)O)CC3)CNC(=O)CCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2 ; O=C(O)C(F)(F)F]

Step 1: Into a 40-mL vial, was placed a mixture of 3-((tert-butoxycarbonyl)amino) propanoic acid (500 mg, 0.998 Eq, 2.64 mmol) in DMF (5 mL), then HATU (4.53 g, 4.50 Eq, 11.9 mmol) and DIEA (1.71 g, 2.30 mL, 5.00 Eq, 13.2 mmol) were added. The mixture was stirred at 25° C. for 10 mins then methyl 3-amino-2-(aminomethyl) propanoate (350 mg, 1 Eq, 2.65 mmol)_{w} as added. The resulting mixture was stirred at 25° C. for 1 hour. The mixture was directly purified by Prep-HPLC using the following conditions: C18 120 g, Spherical 20-40 μm; Mobile phase, Water (0.05% TFA) and ACN (5% ACN to 5% ACN in 1 min, 5% ACN up to 98% in 6 min, 98% ACN to 98% in 3 min); Total flow rate, 70 mL/min; Detector, UV 220 nm. The collected fractions were dried by lyophilization to provide methyl 3-(3-((tert-butoxycarbonyl)amino) propanamido)-2-((3-((tert-butoxycarbonyl)amino) propanamido)methyl) propanoate (700 mg, 1.3 mmol, 50%, 90% Purity) as a colorless oil. Calc'd for C₂₁H₃₈N₄O₈:474.27, found [M+H]: 475.5.

Step 2: Into a 40-mL vial, was placed a mixture of methyl 3-(3-((tert-butoxycarbonyl)amino) propanamido)-2-((3-((tert-butoxycarbonyl)amino) propanamido)methyl) propanoate (700 mg, 1 Eq, 1.48 mmol), LiOH (177 mg, 5.01 Eq, 7.39 mmol), MeOH (9 mL) and water (1.8 mL). The reaction mixture was stirred at 25° C. for 1 hour. The reaction mixture was concentrated under reduced pressure to remove most of the MeOH, then the residue was diluted with water (50 mL). The pH value was adjusted to 6.0 by addition of a saturated NaHSO+solution, then the aqueous solution was extracted with DCM (50 mL×3), dried over anhydrous Na₂SO₄, concentrated under reduced pressure, then lyophilized to afford the title product (600 mg, 1.2 mmol, 84%, 95% purity) as an off-white solid, which was used directly for next step without any purification. Calc'd for C₂₀H₃₆N₄O₈:460. 25, found [M+H]]: 461.3.

Step 3: Into an 8-mL vial, was placed a mixture of 3-(3-((tert-butoxycarbonyl)amino) propanamido)-2-((3-((tert-butoxycarbonyl)amino) propanamido)methyl) propanoic acid (67 mg, 1 Eq, 0.15 mmol), HATU (66 mg, 1.2 Eq, 0.17 mmol) and DIEA (56 mg, 75 μL, 3.0 Eq, 0.43 mmol) in DMF (1 mL). The reaction mixture was stirred at 25° C. for 10 mins then (8-(3-aminophenyl)-1-(3,5-dichlorophenyl)-7-methoxy-1,4-dihydrochromeno[4,3-c]pyrazol-3-yl) (3,3-dimethylmorpholino) methanone (100 mg, 1.2 Eq, 173 μmol)_{w} as added. The mixture was stirred at 25° C. for 1 hour. The crude product was purified by Prep-HPLC under the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide di-tert-butyl(((2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl) propane-1,3-diyl)bis(azanediyl))bis(3-oxopropane-3, 1-diyl))dicarbamate (100 mg, 92 μmol, 63%, 94% purity) as a white solid. Calc'd for C₅₀H₆₂C₁₂N&On: 1020.39, found [M+H]: 1021.3, 1023.3.

Step 4: Into an 8-mL vial, was placed a mixture of di-tert-butyl(((2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl) propane-1,3-diyl)bis(azanediyl))bis(3-oxopropane-3,1-diyl))dicarbamate (100 mg, 1 Eq, 97.8 μmol) and DCM (1.5 mL), to which was added TFA (0.01 mL). The reaction mixture was stirred at 25° C. for 40 min. The mixture was concentrated under reduced pressure to provide N,N′-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl) propane-1,3-diyl)bis(3-aminopropanamide)bis(2,2,2-trifluoroacetate) (100 mg, 87 μmol, 89%, 91% Purity) as a light yellow oil. Calc'd for C₄₄H₄₈C₁₂F6N&On: 1048.27, found [M+H−2TFA]⁺: 821.3, 823.3.

Step 5: Into an 8-mL vial, was placed a mixture of N,N′-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl) propane-1,3-diyl)bis(3-aminopropanamide)bis(2,2,2-trifluoroacetate) (100 mg, 1 Eq, 95.3 μmol) and DMF (1 mL), to which was added DIEA (80 mg, 0.11 mL, 6.5 Eq, 0.62 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (200 mg, 4.19 Eq, 399 μmol). The reaction mixture was stirred at 25° C. for 2 hours. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 10 min; Flow rate: 20 mL/min; Wave Length: 220 nm. The collected fractions were dried by lyophilization to provide the title compound (100 mg, 56 μmol, 58%, 95% purity) as a white solid. Calc'd for C₇₄H₉₉C₁₂F3N₁₆O23:1706.64, found [M+H−TFA]⁺: 1594.0, 1595.6.

### Example 36:2,2′,2″-(10-(2-((1-(2-((3-((3-(1-(3,5-dichlorophenyl)-3-(3,3dimethylmorpholine-4-carbonyl)-7-methoxy-1,4-dihydrochromeno|4,3-clpyrazol-8-yl)phenyl)amino)-3-oxopropyl)amino)-2-oxoethyl) piperidin-4-yl)amino)-2-oxoethyl)-1,4,7,10-tetraazacyclododecane-1,4,7-triyl)triacetic acid (Compound 36)

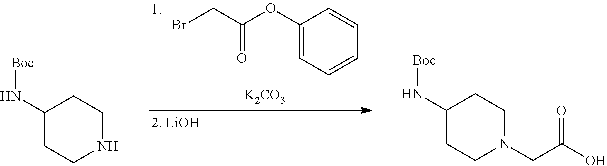 [SMILES: CC(C)(C)OC(=O)NC1CCNCC1 ; O=C(CBr)Oc1ccccc1 ; CC(C)(C)OC(=O)NC1CCN(CC(=O)O)CC1]

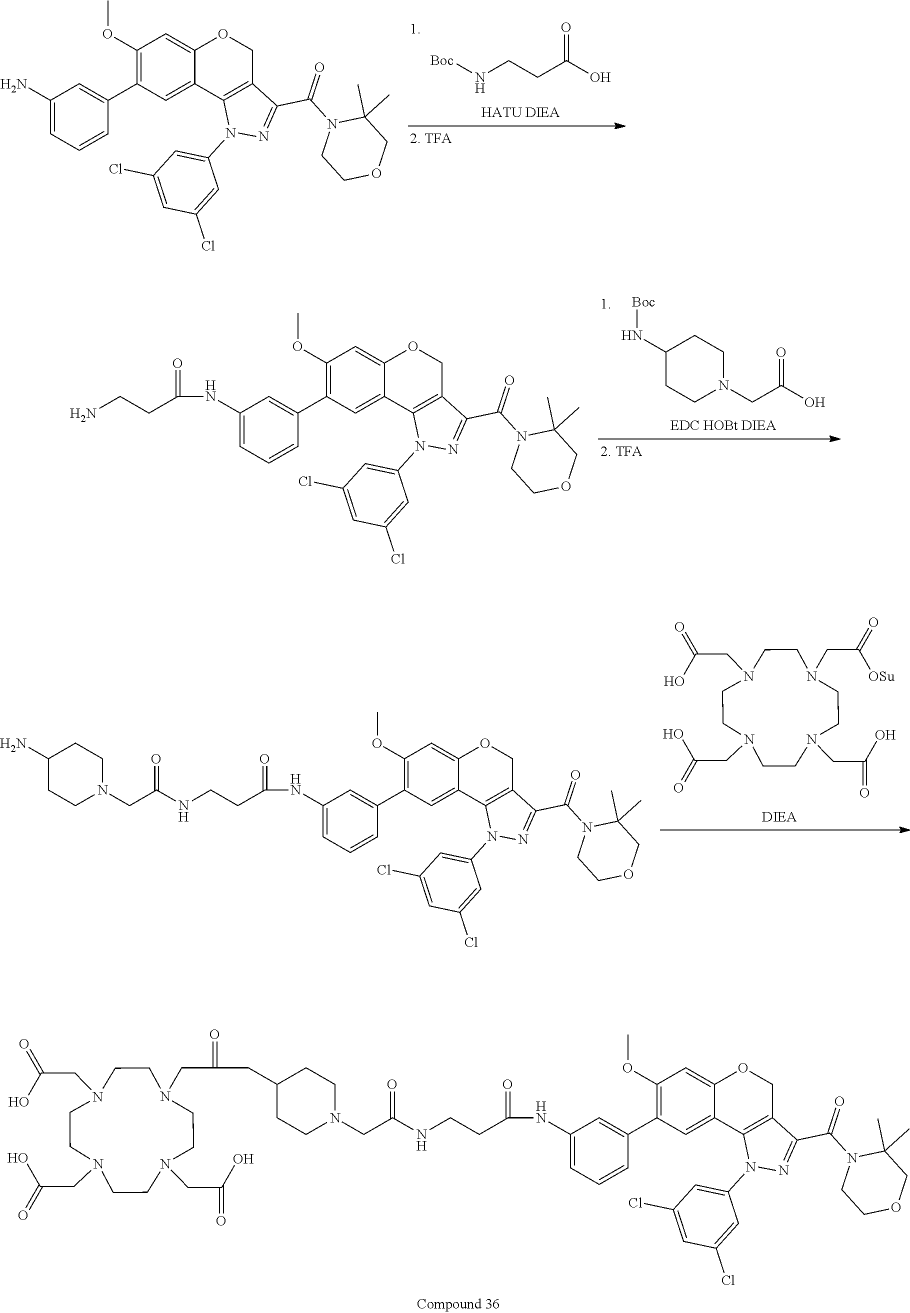

Step 1: Into a 100-mL round bottom flask, was placed a mixture of tert-butyl piperidin-4-ylcarbamate (1.0 g, 1.2 Eq, 5.0 mmol), phenyl 2-bromoacetate (1.8 g, 2.0 Eq, 8.4 mmol), K₂CO₃ (0.58 g, 1.0 Eq, 4.2 mmol) and ACN (10 mL). The reaction mixture was stirred at 25° C. for 4 hours. The crude product was purified by MPLC using the following conditions: Silica gel column 40 g, PE/EtOAc system, the ratio of EtOAc from 0% to 85% in 15 min, Flow rate: 40 mL/min; Wave Length: 254 nm. This resulted in phenyl 2-(4-((tert-butoxycarbonyl)amino) piperidin-1-yl)acetate (1.3 g, 3.9 mmol, 93%) as a white solid. [M+H]-335.2. Calc'd for C18H₂₆N₂O₄:334.19, found [M+H]⁺: 335.2.

Step 2: Into a 40-mL vial, was placed a mixture of phenyl 2-(4-((tert-butoxycarbonyl)amino) piperidin-1-yl)acetate (1.3 g, 1 Eq, 3.9 mmol), LiOH (0.47 g, 5.0 Eq, 20 mmol), MeOH (9 mL) and H₂O(3 mL). The reaction mixture was stirred at 25° C. for 3 hours. The reaction mixture was concentrated under reduced pressure to remove most of the MeOH, the residue was diluted with water (50 mL), the pH value was adjusted to 6.0 by addition of a saturated NaHSO₄ solution, extracted with DCM (50 mL×3), dried over anhydrous Na₂SO₄, and concentrated under reduced pressure to afford 2-(4-((tert-butoxycarbonyl)amino) piperidin-1-yl)acetic acid (800 mg, 3.10 mmol, 80%) as a light yellow oil, which was used directly for next step without any purification. Calc'd for C₁₂H₂₂N₂O₄:258.16, found [M+H]: 259.1.

Step 3: Into a 40-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (400 mg, 1 Eq, 690 μmol), HATU (394 mg, 1.50 Eq, 1.04 mmol), DIEA (268 mg, 361 μL, 3.00 Eq, 2.07 mmol) and DMF (4 mL). The reaction mixture was stirred at 20° C. for 10 minutes, then 3-((tert-butoxycarbonyl)amino) propanoic acid (170 mg, 1.30 Eq, 898 μmol)_{w} as added and the reaction mixture was stirred at 25° C. for an additional 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl) carbamate (387 mg, 516 μmol, 74.7%) as a yellow oil. Calc'd for C₃₈H₄₁C₁₂N₅O₇:749.24, found [M+H]: 750.2.

Step 4: Into a 40-mL vial, was placed a mixture of tert-butyl(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl) carbamate (380 mg, 1 Eq, 506 μmol) and DCM (20 mL), to which was added TFA (1 mL). The reaction mixture was stirred at 25° C. for 40 mins. The mixture were concentrated under reduced pressure to provide 3-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide as a white solid. Calc'd for C₃₃H₃₃C₁₂N_{505:649.19}, found [M+H]: 651.1.

Step 5: Into a 40-mL vial, was placed a mixture of 2-(4-((tert-butoxycarbonyl)amino) piperidin-1-yl)acetic acid (from Step 2; 178 mg, 2.99 Eq, 689 μmol), HOBt (112 mg, 3.17 Eq, 731 μmol), EDC (141 mg, 3.19 Eq, 736 μmol), DIEA (149 mg, 201 μL, 5.00 Eq, 1.15 mmol) and DMF (1.5 mL). The reaction mixture was stirred at 25° C. for 10 minutes, then 3-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (150 mg, 1 Eq, 231 μmol)_{w} as added and the reaction mixture was stirred at 25° C. for an additional 2 hours. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide tert-butyl(1-(2-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-2-oxoethyl) piperidin-4-yl) carbamate (100 mg, 112 μmol, 48.7%) as a yellow oil. Calc'd for C₄₅H₅₃C₁₂N₇O₈:889.33, found [M+H]: 890.3.

Step 6: Into a 40-mL vial, was placed a mixture of tert-butyl(1-(2-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-2-oxoethyl) piperidin-4-yl) carbamate (100 mg, 1 Eq, 112 μmol) and DCM (5 mL), to which was added TFA (0.25 mL). The reaction mixture was stirred at 25° C. for 40 mins. The mixture was concentrated under reduced pressure to provide 3-(2-(4-aminopiperidin-1-yl)acetamido)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (100 mg, 63 μmol, 56%, 50% Purity) as a white solid. Calc'd for C₄₀H₄₅C₁₂N₇O₆:789.28, found [M+H]: 790.2.

Step 7: Into a 40-mL vial, was placed a mixture of 3-(2-(4-aminopiperidin-1-yl)acetamido)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (100 mg, 50% Wt, 1 Eq, 63.2 μmol), DIEA (48.9 mg, 65.9 μL, 5.98 Eq, 378 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (64.1 mg, 2.02 Eq, 128 μmol) and DMF (1 mL). The reaction mixture was stirred at 25° C. for 2 hours. Purification provided 2,2′,2″-(10-(2-((1-(2-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-2-oxoethyl) piperidin-4-yl)amino)-2-oxoethyl)-1,4,7,10-tetraazacyclododecane-1,4,7-triyl)triacetic acid (31.9 mg, 27.1 μmol, 42.9%) as a yellow oil. Calc'd for C₅₆H₇₁C₁₂N₁₁O₁₃:1175.46, found [M+H]: 1176.6, 1178.5.

### Example 37: (R)-2,2′,2″-(10-(2-((1-((3-((3-(1-(3,5-dichlorophenyl)-3-(3,3dimethylmorpholine-4-carbonyl)-7-methoxy-1,4-dihydrochromeno|4,3-clpyrazol-8-yl)phenyl)amino)-3-oxopropyl)amino)-1-oxo-3-sulfopropan-2-yl)amino)-2-oxoethyl)-1,4,7,10-tetraazacyclododecane-1,4,7-triyl)triacetic acid (Compound 37)

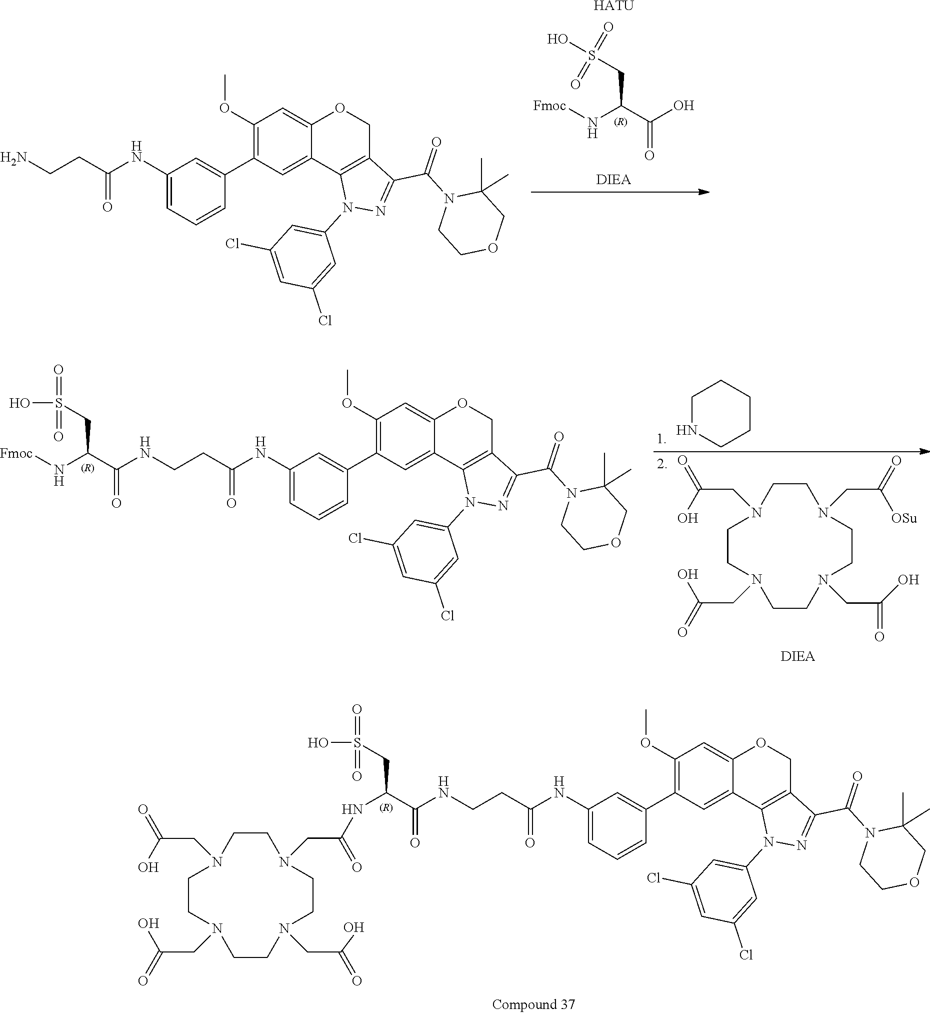

Step 1: Into an 8-mL vial, was placed a mixture of (((9H-fluoren-9-yl) methoxy) carbonyl) (sulfo)-D-alanine (109 mg, 1.21 Eq, 278 μmol), HATU (175 mg, 2.00 Eq, 460 μmol), DIEA (180 mg, 243 μL, 6.04 Eq, 1.39 mmol) and DMF (1.5 mL). The reaction mixture was stirred at 20° C. for 10 minutes, then 3-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) propanamide (150 mg, 1 Eq, 231 μmol)_{w} as added and the reaction mixture was stirred at 25° C. for an additional 2 hours. The mixture was directly purified by MPLC with the following conditions: Column, WelFlash™, C18 120 g, Spherical 20-40 μm; Mobile phase, Water (0.05% NH₃.H₂O) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide (R)-2-((((9H-fluoren-9-yl) methoxy) carbonyl)amino)-3-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-3-oxopropane-1-sulfonic acid (90 mg, 88 μmol, 38%) as a yellow oil. Calc'd for C₅₁H₄₈C₁₂N₆OnS: 1022.25, found [M+H]⁺: 1023.1.

Step 2: Into an 8-mL vial, was placed a mixture of (R)-2-((((9H-fluoren-9-yl) methoxy) carbonyl)amino)-3-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-3-oxopropane-1-sulfonic acid (80 mg, 1 Eq, 78 μmol), piperidine (75 mg, 87 μL, 11 Eq, 0.88 mmol) and DMF (1.0 mL). The reaction mixture was stirred at 25° C. for an additional 2 hours. The crude product was purified by Prep-HPLC using the following conditions: Column: SunFire prep OBD 19*150 mm 5 μm; Mobile Phase A: Water (0.05% NH₃.H₂O); Mobile Phase B: ACN; Gradient: 25% B to 65% B in 8 min; Flow rate: 20 mL/min; Wave Length: 220 nm. The collected fractions were dried by lyophilization to provide (R)-2-amino-3-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-3-oxopropane-1-sulfonic acid (40 mg, 50 μmol, 64%) as an off-white solid. Calc'd for C₃₆H₃₈C₁₂N₆OS: 800.18, found [M+H]: 801.2.

Step 3: Into an 8-mL vial, was placed a mixture of (R)-2-amino-3-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-3-oxopropane-1-sulfonic acid (45 mg, 1 Eq, 56 μmol), DIEA (45 mg, 61 μL, 6.2 Eq, 0.35 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (85 mg, 3.0 Eq, 0.17 mmol) and DMF (1.0 mL). The reaction mixture was stirred at 25° C. for an additional 2 hours. The crude product was purified by Prep-HPLC using the following conditions: Column: SunFire prep OBD 19*150 mm 5 μm; Mobile Phase A: Water (0.05% NH₃.H₂O); Mobile Phase B: ACN; Gradient: 25% B to 65% B in 8 min; Flow rate: 20 mL/min; Wave Length: 220 nm. The collected fractions were dried by lyophilization to provide (R)-2,2′,2″-(10-(2-((1-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropyl)amino)-1-oxo-3-sulfopropan-2-yl)amino)-2-oxoethyl)-1,4,7,10-tetraazacyclododecane-1,4,7-triyl)triacetic acid (21.9 mg, 18.4 μmol, 33%) as an off-white solid. Calc'd for C₅₂H₆₄C₁₂N₁₀O₁₆S: 1186.36, found [M+H]: 1187.6.

### Example 38:2,2′,2″-(10-(2-(((3R,5R)-7-((3-(1-(3,5-dichlorophenyl)-3-(3,3dimethylmorpholine-4-carbonyl)-7-methoxy-1,4-dihydrochromeno|4,3-c]pyrazol-8-yl)phenyl)amino)-3,5-dihydroxy-7-oxoheptyl)amino)-2-oxoethyl)-1,4,7,10-tetraazacyclododecane-1,4,7-triyl)triacetic acid (Compound 38)

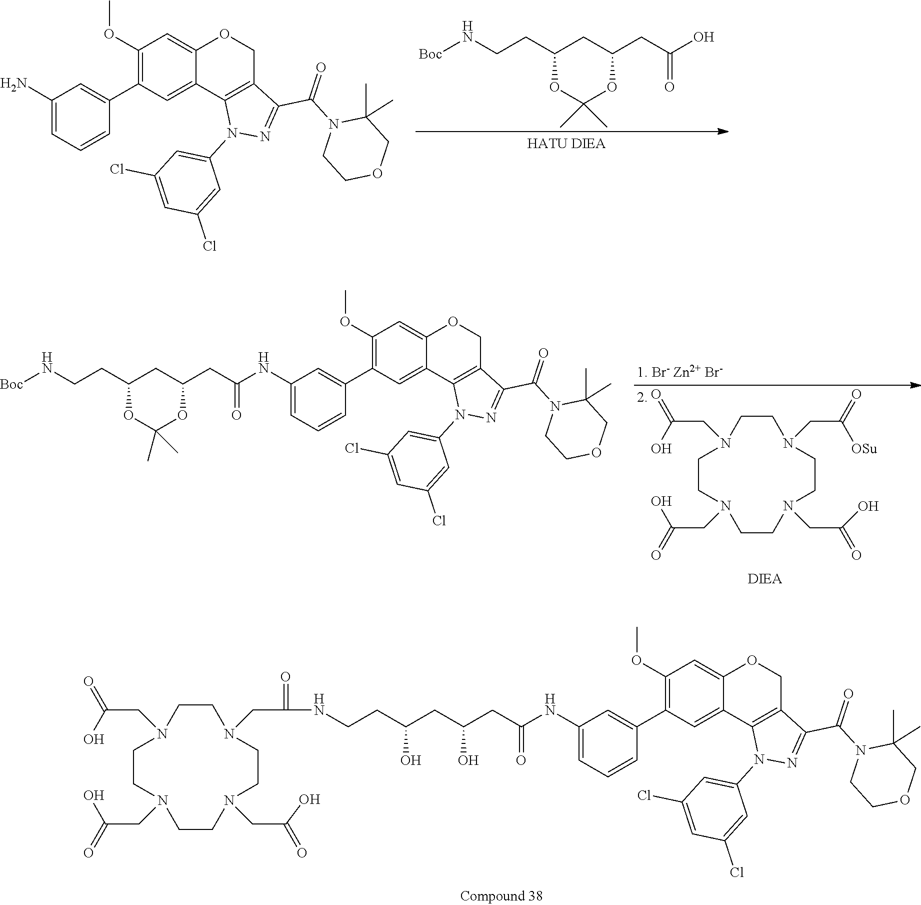

Step 1: Into a 40-mL vial, was placed a mixture of 2-((4R,6R)-6-(2-((tert-butoxycarbonyl)amino)ethyl)-2,2-dimethyl-1,3-dioxan-4-yl)acetic acid (120 mg, 1.46 Eq, 378 μmol), HATU (129 mg, 1.31 Eq, 339 μmol), DIEA (208 mg, 280 μL, 6.22 Eq, 1.61 mmol) and DMF (1.5 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (150 mg, 1 Eq, 259 μmol)_{w} as added and the reaction mixture was stirred at 25° C. for an additional 2 hours. The mixture was directly purified by MPLC using the following conditions: Column, WelFlash™, C18 120 g, Spherical 20-40 μm; Mobile phase, water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(2-((4R,6R)-6-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl)-2,2-dimethyl-1,3-dioxan-4-yl)ethyl) carbamate (116 mg, 132 μmol, 51.0%) as an off-white solid. Calc'd for C₄₅H₅₃Cl2N₅O₉:877.32, found [M+H]⁺: 878.3.

Step 2: Into a 8-mL vial, was placed a mixture of tert-butyl(2-((4R,6R)-6-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl)-2,2-dimethyl-1,3-dioxan-4-yl)ethyl) carbamate (100 mg, 1 Eq, 114 μmol), zinc bromide (56 mg, 13 μL, 2.2 Eq, 0.25 mmol) and DCM (1.0 mL). The reaction mixture was stirred at 25° C. for 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide (3R,5R)-7-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,5-dihydroxyheptanamide (55 mg, 74 μmol, 65%) as an off-white solid. Calc'd for C₃₇H₄₁C₁₂N₅O₇:737.24, found [M+H]: 738.2.

Step 3: Into an 8-mL vial, was placed a mixture of (3R,5R)-7-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-3,5-dihydroxyheptanamide (55 mg, 1 Eq, 74 μmol), DIEA (62 mg, 84 μL, 6.4 Eq, 0.48 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (85 mg, 2.3 Eq, 0.17 mmol) and DMF (1.0 mL). The reaction mixture was stirred at 25° C. for an additional 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-(((3R,5R)-7-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,5-dihydroxy-7-oxoheptyl)amino)-2-oxoethyl)-1,4,7,10-tetraazacyclododecane-1,4,7-triyl)triacetic acid (45.2 mg, 40.2 μmol, 54%) as an off-white solid. Calc'd for C₅₃H₆₇C₁₂N₉O₁₄:1123.42, found [M+H]: 1124.4.

### Example 39: (E)-2,2′,2″-(10-(2-((2-(((3-(1-(3,5-dichlorophenyl)-3-(3,3-dimethylmorpholine-4-carbonyl)-7-methoxy-1,4-dihydrochromeno|4,3-c]pyrazol-8-yl)benzylidene)amino) oxy)ethyl)amino)-2-oxoethyl)-1,4,7,10-tetraazacyclododecane-1,4,7-triyl)triacetic acid (Compound 39)

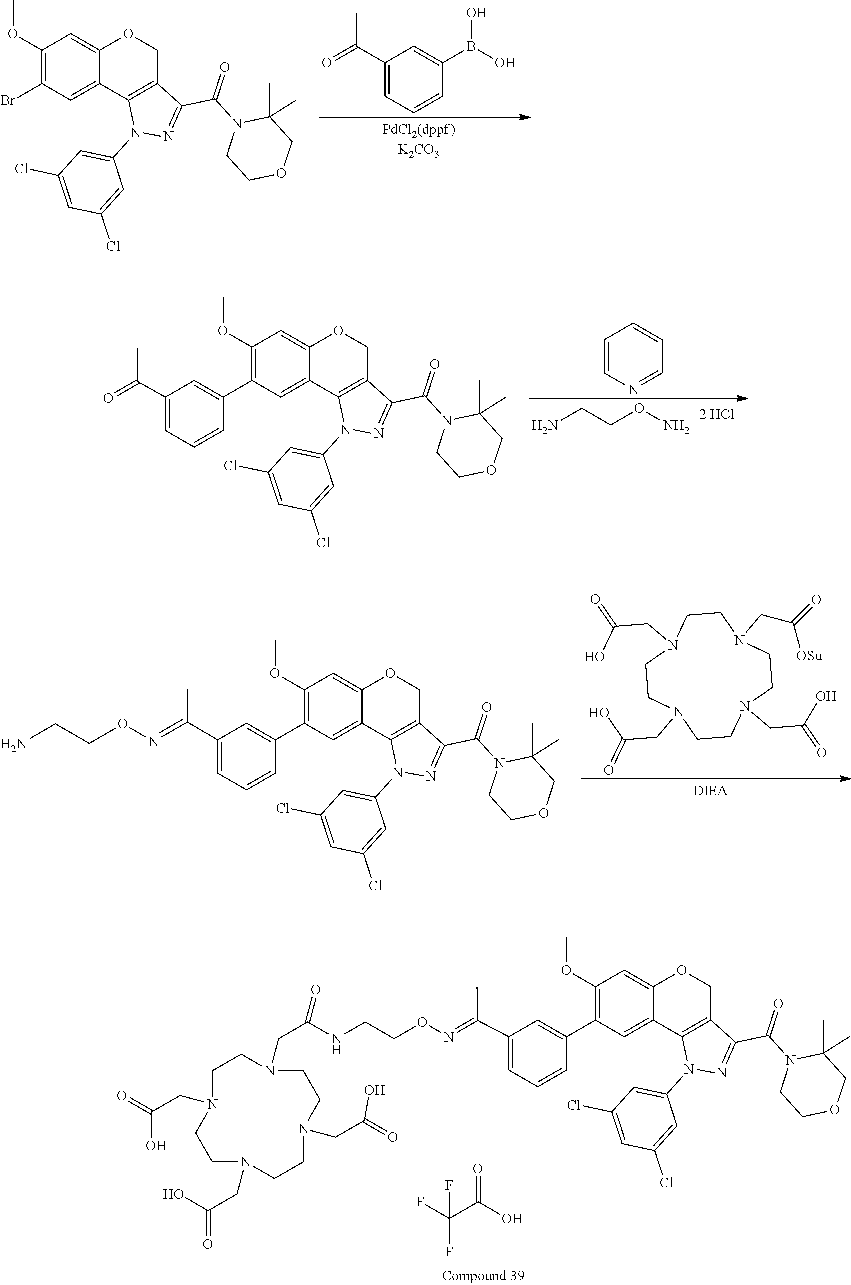

Step 1: Into a 40-mL vial, was placed a mixture of (8-bromo-1-(3,5-dichlorophenyl)-7-methoxy-1,4-dihydrochromeno[4,3-c]pyrazol-3-yl) (3,3-dimethylmorpholino) methanone (1.5 g, 1 Eq, 2.6 mmol), (3-acetylphenyl) boronic acid (480 mg, 1.1 Eq, 2.93 mmol), K₂CO₃ (1.1 g, 3.0 Eq, 8.0 mmol), PdCl2 (dppf) (97 mg, 0.050 Eq, 0.13 mmol), 1,4-dioxane (15 mL) and water (1.5 mL) and the reaction mixture was stirred at 100° C. for 0.5 hour under N₂. The mixture was concentrated under reduced pressure. The crude product was purified by MPLC with the following conditions: Silica gel column 40 g, PE/EtOAc system, the ratio of EtOAc from 0% to 30% in 30 min, Flow rate: 40 mL/min; Wave Length: 254 nm. The collected fractions were concentrated to provide 1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) ethan-1-one (1.26 g, 1.9 mmol, 71%, 90% Purity) as a white solid. Calc'd for C₃₂H₂₉C₁₂N₃O₅:605.15, found [M+H]⁺: 606.2, 608.2.

Step 2: Into a 40-mL vial, was placed a mixture of 1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) ethan-1-one (1.1 g, 1 Eq, 1.8 mmol), 2-(aminooxy)ethan-1-amine dihydrochloride (410 mg, 1.5 Eq, 2.75 mmol), pyridine (43 mg, 44 μL, 0.30 Eq, 0.54 mmol) and EtOH (11 mL). The reaction mixture was stirred at 90° C. for 3 hours. To the mixture was added PE (50 ml) and the mixture was filtered to provide (E)-(8-(3-(1-((2-aminoethoxy)imino) ethyl)phenyl)-1-(3,5-dichlorophenyl)-7-methoxy-1,4-dihydrochromeno[4,3-c]pyrazol-3-yl) (3,3-dimethylmorpholino) methanone (800 mg, 0.96 mmol, 53%, 80% Purity) as a white solid. Calc'd for C₃₄H₃₅C₁₂N₂O₅:663.20, found [M+H]: 664.4, 666.4.

Step 3: Into an 8-mL vial, was placed a mixture of (E)-(8-(3-(1-((2-aminoethoxy)imino) ethyl)phenyl)-1-(3,5-dichlorophenyl)-7-methoxy-1,4-dihydrochromeno[4,3-c]pyrazol-3-yl) (3,3-dimethylmorpholino) methanone (60 mg, 1 Eq, 90 μmol) in DMF (1 mL), then DIEA (35 mg, 47 μL, 3.0 Eq, 0.27 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (91 mg, 2.0 Eq, 0.18 mmol) were added. The resulting mixture was stirred at 25° C. for 2 hours. The crude product was purified by Prep-HPLC with the following conditions: Column: SunFire prep OBD 30*150 mm 5 μm; Mobile Phase A: Water (0.1% TFA); Mobile Phase B: ACN; Gradient: 10% B to 25% B 25% in 8 min, 25% B to 25% B in 1 min; Flow rate: 60 mL/min; Wave Length: 220 nm to afford two isomers, the front peak fractions were dried by lyophilization to afford (E)-2,2′,2″-(10-(2-((2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)amino)-2-oxoethyl)-1,4,7,10-tetraazacyclododecane-1,4,7-triyl)triacetic acid--2,2,2-trifluoroacetic acid (1/1) (59.8 mg, 50 μmol, 55%, 97% Purity) as a white solid. Calc'd for C₅₂H₆₂C₁₂F3N₉O₁₄:1163. 37, found [M+H]⁺: 1050.7, 1052.7.

### Example 40: (E)-2,2′,2″-(10-(2-(4-(2-((2-(((3-(1-(3,5-dichlorophenyl)-3-(3,3dimethylmorpholine-4-carbonyl)-7-methoxy-1,4-dihydrochromeno|4,3-c]pyrazol-8-yl)benzylidene)amino) oxy)ethyl)amino)-2-oxoethyl) piperazin-1-yl)-2-oxoethyl)-1,4,7,10-tetraazacyclododecane-1,4,7-triyl)triacetic acid (Compound 40)

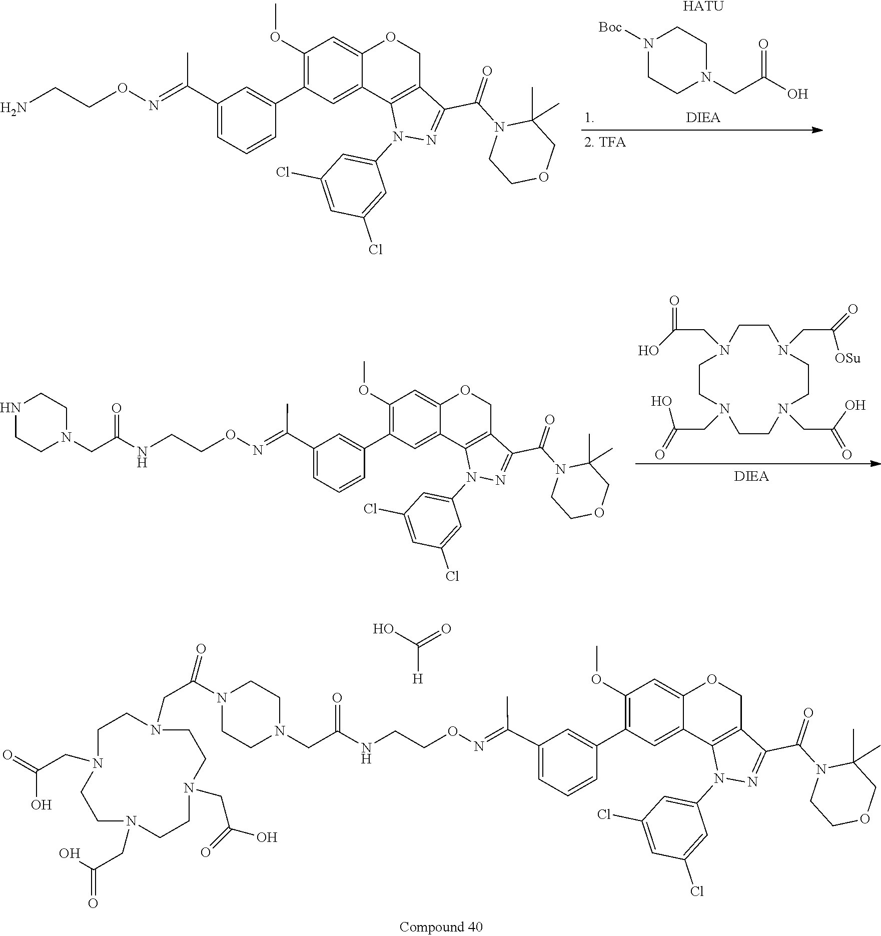

Step 1: Into an 8-mL vial, was placed a mixture of (E)-(8-(3-(1-((2-aminoethoxy)imino) ethyl)phenyl)-1-(3,5-dichlorophenyl)-7-methoxy-1,4-dihydrochromeno[4,3-c]pyrazol-3-yl) (3,3-dimethylmorpholino) methanone (from Example 39, Step 2; 100 mg, 1 Eq, 150 μmol) and DMF (1 mL), to which was added HATU (130 mg, 2.27 Eq, 342 μmol), 2-(4-(tert-butoxycarbonyl) piperazin-1-yl)acetic acid (75 mg, 2.0 Eq, 0.31 mmol) and DIEA (95 mg, 0.13 mL, 4.9 Eq, 0.74 mmol). The reaction mixture was stirred at 25° C. for 16 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated to afford tert-butyl (E)-4-(2-((2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)amino)-2-oxoethyl) piperazine-1-carboxylate (40 mg, 45 μmol, 30%) as a yellow solid. Calc'd for C₄₅H₅₃C₁₂NO_{8:889.33}, found [M+H]⁺: 890.2, 892.2.

Step 2: Into an 8 mL flask was added a mixture of tert-butyl(E)-4-(2-((2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)amino)-2-oxoethyl) piperazine-1-carboxylate (40 mg, 1 Eq, 45 μmol), TFA (0.1 mL) and DCM (0.5 mL).

The mixture was stirred for 1 hour at 26° C. then the mixture was concentrated under reduced pressure to provide (E)-N-(2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)-2-(piperazin-1-yl)acetamide (40 mg, 45 μmol, 100%, 89% Purity) as a brown oil, which was used directly in the next step without any purification. Calc'd for C₄₀H₄₅C₁₂N₇O₆:789.28, found [M+H]: 790.5, 792.5.

Step 3: Into an 8-mL vial, was placed a mixture of (E)-N-(2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)-2-(piperazin-1-yl)acetamide (40 mg, 89% Wt, 1 Eq, 45 μmol) and DMF (0.5 mL), to which was added DIEA (40 mg, 54 μL, 6.9 Eq, 0.31 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (80 mg, 3.5 Eq, 0.16 mmol). The reaction mixture was stirred at 27° C. for 2 hours. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 65% B in 10 min; Flow rate: 20 mL/min; Wave Length: 220 nm. The collected fractions were dried by lyophilization to provide (E)-2,2′,2″-(10-(2-(4-(2-((2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)amino)-2-oxoethyl) piperazin-1-yl)-2-oxoethyl)-1,4,7,10-tetraazacyclododecane-1,4,7-triyl)triacetic acid--formic acid (1/1) (17.7 mg, 14.5 μmol, 32%) as a white solid. Calc'd for C₅₇H₇₃C₁₂N₁₁O₁₅:1221.47, found [M+H−FA]]: 1176.5, 1178.5.

### Example 41: (E)-2,2′,2″-(10-(1-(3-(1-(3,5-dichlorophenyl)-3-(3,3-dimethylmorpholine-4carbonyl)-7-methoxy-1,4-dihydrochromeno|4,3-c|pyrazol-8-yl)phenyl)-7,16-dioxo-3,9,12-trioxa-2,6,15-triazaheptadec-1-en-17-yl)-1,4,7,10-tetraazacyclododecane-1,4,7-triyl)triacetic acid (Compound 41)

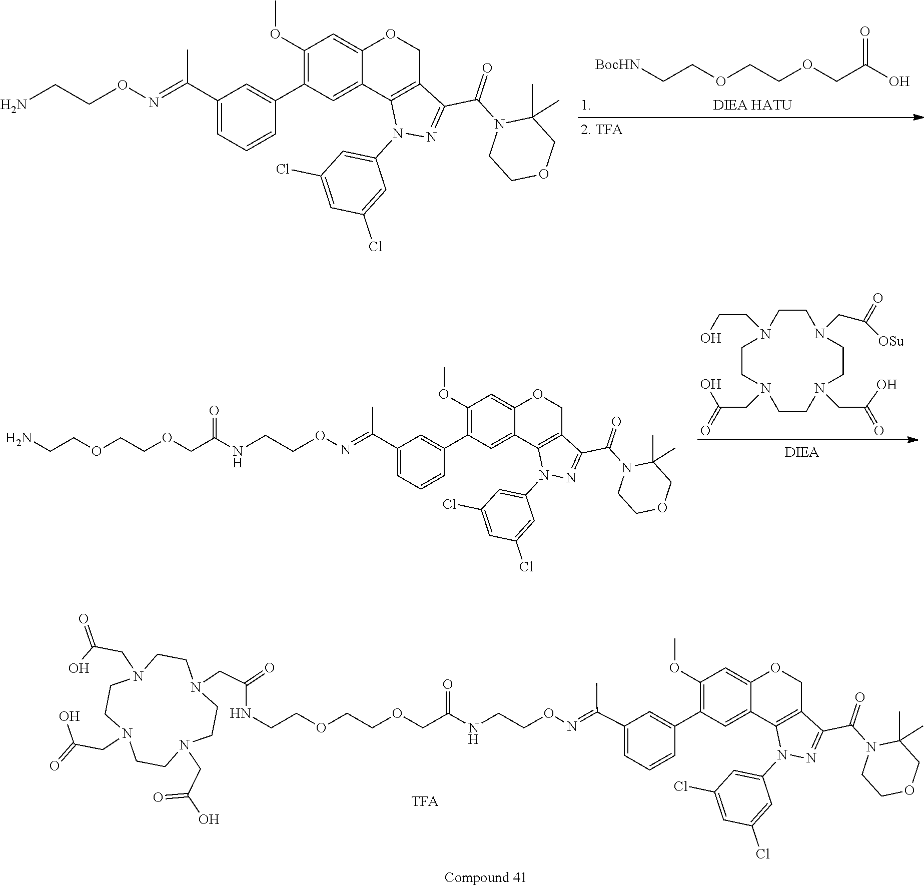

Step 1: Into an 8-mL vial, was placed a mixture of 2,2-dimethyl-4-oxo-3,8, 11-trioxa-5-azatridecan-13-oic acid (90 mg, 1.1 Eq, 0.34 mmol), DIEA (120 mg, 162 μL, 3.09 Eq, 928 μmol), HATU (130 mg, 1.14 Eq, 342 μmol) and DMF (2 mL). The reaction mixture was stirred at 20° C. for 10 minutes, then (E)-(8-(3-(1-((2-aminoethoxy)imino) ethyl)phenyl)-1-(3,5-dichlorophenyl)-7-methoxy-1,4-dihydrochromeno[4,3-c]pyrazol-3-yl) (3,3-dimethylmorpholino) methanone (from Example 39, Step 2; 200 mg, 1 Eq, 301 μmol)_{w} as added and the reaction mixture was stirred at 25° C. for an additional 1 hour. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided tert-butyl(E)-(2-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-8-oxo-4,10,13-trioxa-3,7-diazapentadec-2-en-15-yl) carbamate (150 mg, 165 μmol, 54.8%) as a yellow solid. Calc'd for C₄₅H₅₄C₁₂N₆O₁₀:908.33, found [M+H]⁺: 909.4, 911.4.

Step 2: Into an 8-mL vial, was placed a mixture of tert-butyl(E)-(2-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-8-oxo-4, 10,13-trioxa-3,7-diazapentadec-2-en-15-yl) carbamate (145 mg, 1 Eq, 159 μmol) and DCM (2 mL), to which was added TFA (0.5 mL). The reaction mixture was stirred at 25° C. for 1 hour. The mixture was concentrated under reduced pressure to provide (E)-2-(2-(2-aminoethoxy)ethoxy)-N-(2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)acetamide (150 mg, 0.14 mmol, 87%, 75% Purity) as a yellow oil. Calc'd for C₄₀H₄₆C₁₂N₆O8:808.27, found [M+H]⁺: 809.2, 811.2.

Step 3: Into a 40-mL vial, was placed a mixture of (E)-2-(2-(2-aminoethoxy)ethoxy)-N-(2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)acetamide (150 mg, 1 Eq, 185 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (130 mg, 1.40 Eq, 259 μmol), DIEA (120 mg, 162 μL, 5.01 Eq, 928 μmol) and DMF (1.5 mL). The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide (E)-2,2′,2″-(10-(2-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-8,17-dioxo-4, 10, 13-trioxa-3,7,16-triazaoctadec-2-en-18-yl)-1,4,7,10-tetraazacyclododecane-1,4,7-triyl)triacetic acid--2,2,2-trifluoroacetic acid (1/1) (63.6 mg, 48.5 μmol, 26.2%) as a yellow oil. Calc'd for C₅₈H₇₃C₁₂F3N₁₀O₁₇:1308.44, found [M+H−TFA]⁺: 1195.7, 1197.7.

### Example 42: (E)-2,2′,2″-(10-(1-(3-(1-(3,5-dichlorophenyl)-3-(3,3-dimethylmorpholine-4carbonyl)-7-methoxy-1,4-dihydrochromeno|4,3-c|pyrazol-8-yl)phenyl)-9,12-dimethyl-7,10,13-trioxo-3-oxa-2,6,9,12-tetraazatetradec-1-en-14-yl)-1,4,7,10-tetraazacyclododecane-1,4,7-triyl)triacetic acid (Compound 42)

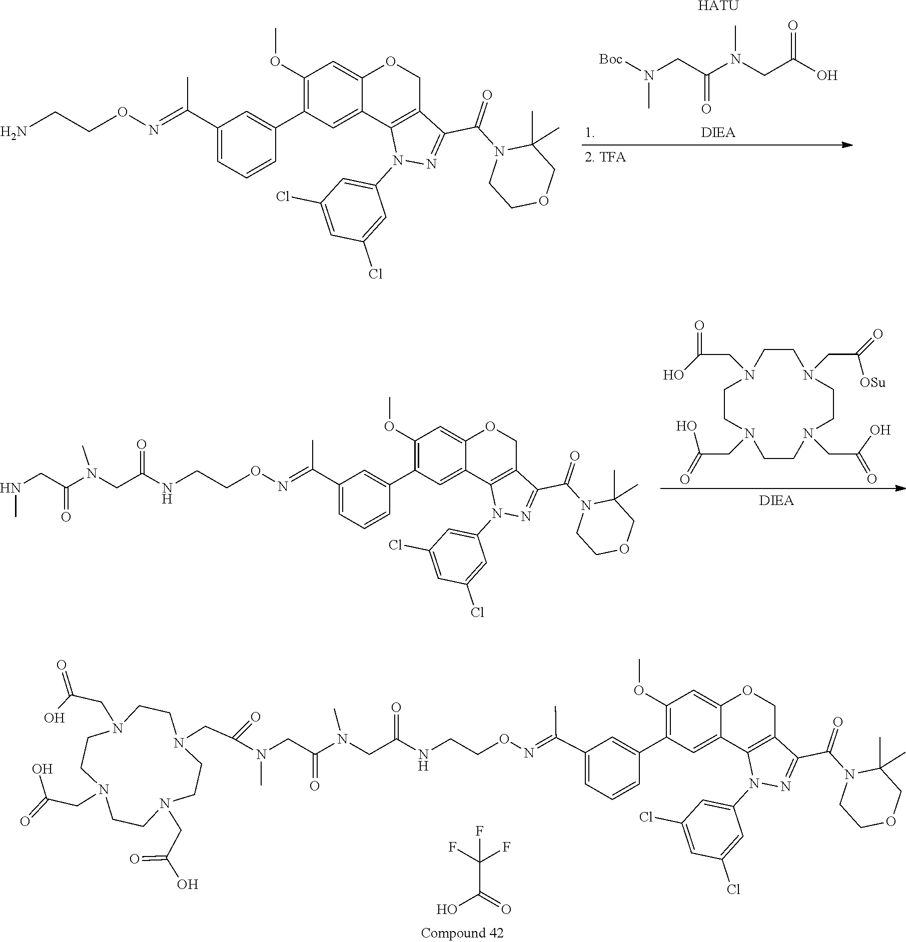

Step 1: Into an 8-mL vial, was placed a mixture of (E)-(8-(3-(1-((2-aminoethoxy)imino) ethyl)phenyl)-1-(3,5-dichlorophenyl)-7-methoxy-1,4-dihydrochromeno[4,3-c]pyrazol-3-yl) (3,3-dimethylmorpholino) methanone (from Example 39, Step 2; 100 mg, 1 Eq, 150 μmol) and DMF (1 mL), to which was added HATU (120 mg, 2.10 Eq, 316 μmol), N—(N-(tert-butoxycarbonyl)-N-methylglycyl)-N-methylglycine (80 mg, 2.0 Eq, 0.31 mmol) and DIEA (90 mg, 0.12 mL, 4.6 Eq, 0.70 mmol). The reaction mixture was stirred at 26° C. for 16 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated to afford tert-butyl(E)-(2-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-10-methyl-8,11-dioxo-4-oxa-3,7, 10-triazadodec-2-en-12-yl)(methyl) carbamate (70 mg, 77 μmol, 51%) as a yellow solid. Calc'd for C₄₅H₅₃C₁₂N₇O₉:905.33, found [M+H]⁺: 906.2, 908.2.

Step 2: Into an 8 mL flask was added a mixture of tert-butyl(E)-(2-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-10-methyl-8, 11-dioxo-4-oxa-3,7,10-triazadodec-2-en-12-yl)(methyl) carbamate (70 mg, 1 Eq, 77 μmol), TFA (0.1 mL) and DCM (0.5 mL). The mixture was stirred for 1 hour at 26° C. The collected fractions were concentrated under reduced pressure to provide (E)-N-(2-((2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)amino)-2-oxoethyl)-N-methyl-2-(methylamino) acetamide (70 mg, 77 μmol, 100%, 89% Purity) as a brown oil, which was used directly for next step without any purification. Calc'd for C₄₀H₄₅C₁₂N—O7: 805.27, found [M+H]⁺: 806.5, 808.5.

Step 3: Into an 8-mL vial, was placed a mixture of (E)-N-(2-((2-(((1-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ethylidene) amino)oxy)ethyl)amino)-2-oxoethyl)-N-methyl-2-(methylamino) acetamide (70 mg, 89% Wt, 1 Eq, 77 μmol) and DMF (1 mL), to which was added DIEA (60 mg, 81 μL, 6.0 Eq, 0.46 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (130 mg, 3.4 Eq, 259 μmol). The reaction mixture was stirred at 25° C. for 2 hours. The crude product was purified by Prep-HPLC using the following conditions: Column: SunFire prep OBD 19*150 mm 5 μm; Mobile Phase A: Water (0.05% TFA); Mobile Phase B: ACN; Gradient: 25% B to 65% B in 10 min; Flow rate: 20 mL/min; Wave Length: 220 nm. The collected fractions were dried by lyophilization to provide (E)-2,2′,2″-(10-(2-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)-10,13-dimethyl-8, 11, 14-trioxo-4-oxa-3,7, 10, 13-tetraazapentadec-2-en-15-yl)-1,4,7,10-tetraazacyclododecane-1,4,7-triyl)triacetic acid--2,2,2-trifluoroacetic acid (1/1) (31 mg, 24 μmol, 31%) as a white solid. Calc'd for C₅₈H₇₂C₁₂F3N₁₁O₁₆:1305.45, found [M+H−TFA]]: 1192.5, 1194.5.

### Example 43:2,2′,2″-(10-(2-((2-((3-(1-(3,5-dichlorophenyl)-3-(3,3-dimethylmorpholine-4carbonyl)-7-methoxy-1,4-dihydrochromeno|4,3-c]pyrazol-8-yl)phenyl)amino)-2-oxoethoxy)amino)-2-oxoethyl)-1,4,7,10-tetraazacyclododecane-1,4,7-triyl)triacetic acid (Compound 43)

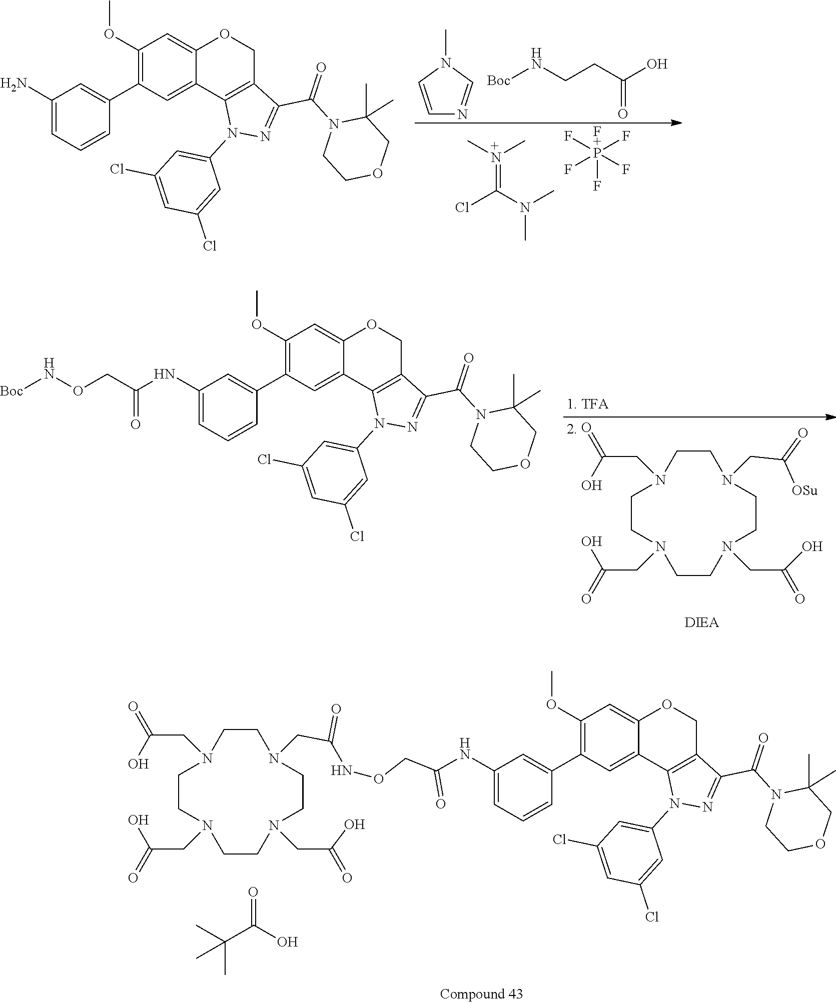

Step 1: Into a 40-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (300 mg, 1 Eq, 518 μmol), 1-Methylimidazole (N—) (85 mg, 82 μL, 2.0 Eq, 1.0 mmol), 2-(((tert-butoxycarbonyl)amino)oxy) acetic acid (150 mg, 1.52 Eq, 785 μmol), TCFH (220 mg, 1.51 Eq, 784 μmol) and DMF (3.5 mL). The reaction mixture was stirred at 25° C. for 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide tert-butyl(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethoxy) carbamate (370 mg, 0.46 mmol, 88%, 93% Purity) as a white solid. Calc'd for C₃₇H₃₉C₁₂N₅O₈:751.22, found [M+H]⁺: 752.2, 754.2, 756.2.

Step 2: Into an 8-mL vial, was placed a mixture of tert-butyl(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethoxy) carbamate (150 mg, 1 Eq, 199 μmol) and DCM (1.5 mL), to which was added TFA (0.1 mL). The reaction mixture was stirred at 25° C. for 5 hours. The mixture was concentrated under reduced pressure. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 2-(aminooxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (65 mg, 0.10 mmol, 50%) as a white solid. Calc'd for C₃₂H₃₁C₁₂N₅O₆:651.16, found [M+H]: 652.2, 654.2.

Step 3: Into an 8-mL vial, was placed a mixture of 2-(aminooxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (65 mg, 1 Eq, 0.10 mmol), DIEA (13 mg, 18 μL, 1.0 Eq, 0.10 mmol) and DMF (1.5 mL), to which was added 2,2′,2″-(10-(2-((2,5-dioxopyrrolidin-1-yl)oxy)-2-oxoethyl)-1,4,7,10-tetraazacyclododecane-1,4,7-triyl)triacetic acid (0.10 g, 2.0 Eq, 0.20 mmol). The reaction mixture was stirred at 25° C. for 2 hours. The mixture was concentrated under reduced pressure. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-((2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethoxy)amino)-2-oxoethyl)-1,4,7,10-tetraazacyclododecane-1,4,7-triyl)triacetic acid--pivalic acid (1/1) (24 mg, 21 μmol, 21%) as a white solid. Calc'd for C₅₃H₆₇C₁₂N₉O₁₅:1139.41, found [M+H−PivOH, M+Na-PivOH]⁺: 1038.2, 1060.2.

### Example 44:2,2′,2″-(10-(2-(3-((2-((3-(1-(3,5-dichlorophenyl)-3-(3,3-dimethylmorpholine-4carbonyl)-7-methoxy-1,4-dihydrochromeno|4,3-c|pyrazol-8-yl)phenyl)amino)-2-oxoethoxy)imino) azetidin-1-yl)-2-oxoethyl)-1,4,7,10-tetraazacyclododecane-1,4,7-triyl)triacetic acid (Compound 44)

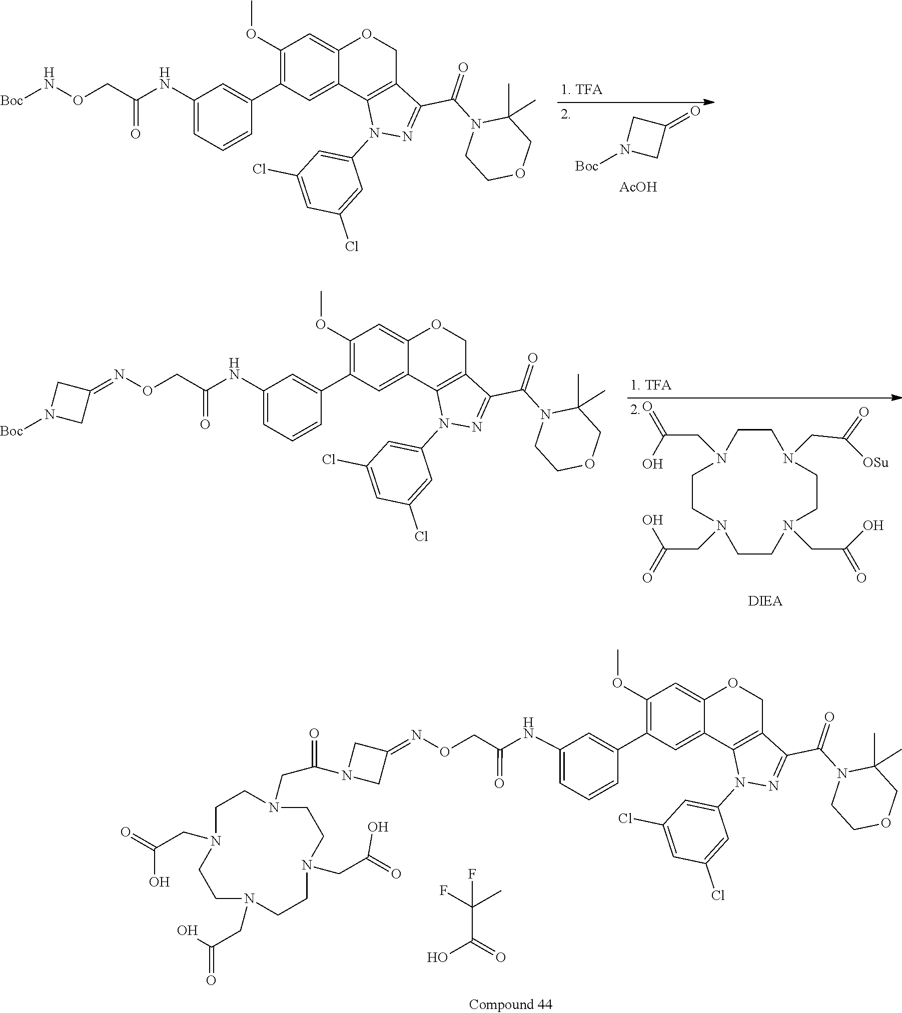

Step 1: Into an 8-mL vial, was placed a mixture of tert-butyl(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethoxy) carbamate (from Example 43, Step 1; 150 mg, 1 Eq, 199 μmol) and DCM (1.5 mL), to which was added TFA (0.3 mL). The reaction mixture was stirred at 25° C. for 15 min. The mixture was concentrated under reduced pressure. The crude product was purified using a C-18 column; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2-(aminooxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (100 mg, 153 μmol, 76.9%) as a white solid. Calc'd for C₃₂H₃₁C₁₂N₅O₆:651.17, found [M+H]⁺: 652.2, 654.2.

Step 2: Into an 8-mL vial, was placed a mixture of 2-(aminooxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (100 mg, 1 Eq, 153 μmol), tert-butyl 3-oxoazetidine-1-carboxylate (40 mg, 1.5 Eq, 0.23 mmol), AcOH (28 mg, 27 μL, 3.0 Eq, 0.47 mmol) and MeOH (1 mL). The reaction mixture was stirred at 40° C. for 16 hours. The mixture was concentrated under reduced pressure. The crude product was purified by Prep-HPLC with the following conditions: Column: C-18 19*150 mm 5 μm; Mobile Phase A: Water (0.05% FA); Mobile Phase B: ACN; Gradient: 25% B to 98% B in 8 min; Flow rate: 70 mL/min; Wave Length: 220 nm. The collected fractions were dried by lyophilization to provide tert-butyl 3-((2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethoxy)imino) azetidine-1-carboxylate (65 mg, 81 μmol, 53%) as a white solid. Calc'd for C₄₀H₄₂C₁₂N₆O₈:804.24 found [M+H]⁺: 805.2, 807.2.

Step 3: Into an 8-mL vial, was placed a mixture of tert-butyl 3-((2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethoxy)imino) azetidine-1-carboxylate (65 mg, 1 Eq, 81 μmol) and DCM (1.0 mL), to which was added TFA (0.2 mL). The reaction mixture was stirred at 25° C. for 15 mins. The mixture was concentrated under reduced pressure to provide 2-((azetidin-3-ylideneamino)oxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (52 mg, 55 μmol, 69%, 75% Purity) as a white solid. Calc'd for C₃₅H₃₄C₁₂N₆O₆:704.19, found [M+H]⁺: 705.1.

Step 4: Into an 8-mL vial, was placed a mixture of 2-((azetidin-3-ylideneamino)oxy)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (52 mg, 1 Eq, 74 μmol), DIEA (29 mg, 39 μL, 3.0 Eq, 0.22 mmol) and DMF (1 mL), to which was added 2,2′,2″-(10-(2-((2,5-dioxopyrrolidin-1-yl)oxy)-2-oxoethyl)-1,4,7,10-tetraazacyclododecane-1,4,7-triyl)triacetic acid (92 mg, 2.5 Eq, 0.18 mmol). The reaction mixture was stirred at 25° C. for 2 hours. The mixture was concentrated under reduced pressure. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-(3-((2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethoxy)imino) azetidin-1-y1)-2-oxoethyl)-1,4,7,10-tetraazacyclododecane-1,4,7-triyl)triacetic acid--2,2,2-trifluoroacetic acid (1/1) (16.8 mg, 13.9 μmol, 19%) as a white solid. Calc'd for C₅₃H₆₁C₁₂F3N₁₀O₁₅:1204.36, found [M+H−TFA]⁺: 1091.6.

### Example 45:2,2′,2″-(10-(2-(4-(N-(17-carboxy-3,6,9,12,15-pentaoxaheptadecyl)-4-((3-(1-(3,5dichlorophenyl)-3-(3,3-dimethylmorpholine-4-carbonyl)-7-methoxy-1,4-dihydrochromeno|4,3-clpyrazol-8-yl)phenyl)amino)-4-oxobutanamido) piperidin-1-yl)-2-oxoethyl)-1,4,7,10-tetraazacyclododecane-1,4,7-triyl)triacetic acid

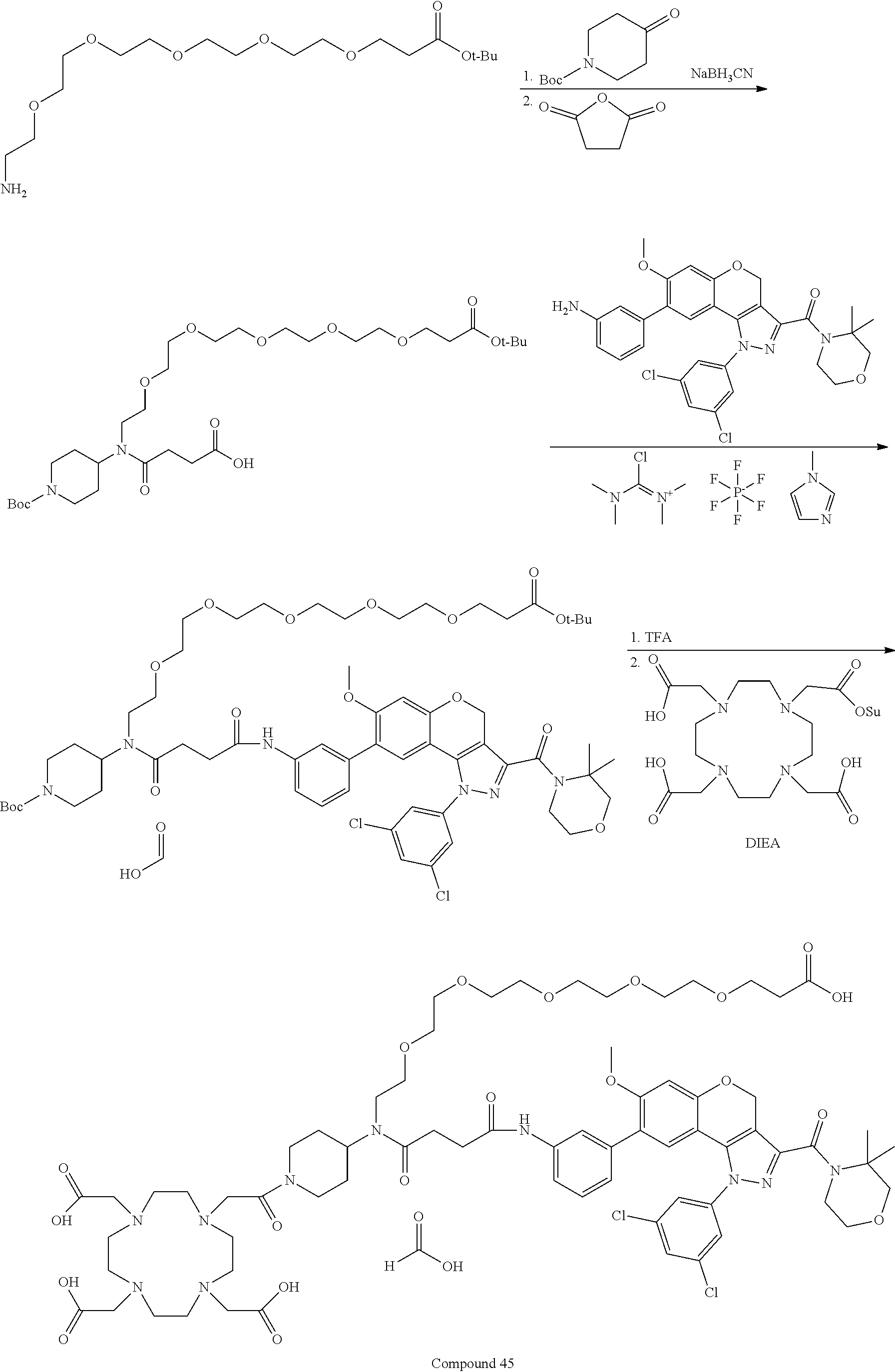

Step 1: Into a 40-mL vial, was placed a mixture of tert-butyl 1-amino-3,6,9,12,15-pentaoxaoctadecan-18-oate (500 mg, 1 Eq, 1.37 mmol), tert-butyl 4-oxopiperidine-1-carboxylate (545 mg, 2.00 Eq, 2.74 mmol) and DCE (10 mL). The reaction mixture was stirred at 20° C. for 50 minutes, then sodium cyanoborohydride (258 mg, 3.00 Eq, 4.11 mmol)_{w} as added and the reaction mixture was stirred at 25° C. for an additional 4 hours. The collected fractions were concentrated under reduced pressure. The crude product was purified by MPLC with the following conditions: Silica gel column 120 g, PE/EtOAc system, the ratio of EtOAc from 0% to 85% in 15 min, Flow rate: 90 mL/min; Wave Length: 254 nm. The collected fractions were concentrated under reduced pressure to provide tert-butyl 4-((20,20-dimethyl-18-oxo-3,6,9,12, 15, 19-hexaoxahenicosyl)amino) piperidine-1-carboxylate (1.35 g, 1.7 mmol, 130%, 70% Purity) as a yellow oil. Calc'd for C₂₇H₅₂N₂O₉:548.37, found [M+H]⁺: 549.5.

Step 2: Into a 40-mL vial, was placed a mixture of tert-butyl 4-((20,20-dimethyl-18-oxo-3,6,9,12, 15, 19-hexaoxahenicosyl)amino) piperidine-1-carboxylate (1.35 g, 1 Eq, 2.46 mmol), dihydrofuran-2,5-dione (492 mg, 2.00 Eq, 4.92 mmol), DMAP (601 mg, 2.00 Eq, 4.92 mmol) and DCE (15 mL). The reaction mixture was stirred at 60° C. for 2 hours. The mixture was diluted with 50 mL of water, extracted with EtOAc (100 mL×3), then the combined organic layers were washed with water (100 mL×2), brine (100 mL), dried over anhydrous Na₂SO₄ and concentrated under reduced pressure. The crude product was purified by MPLC with the following conditions: Silica gel column 80 g, PE/EtOAc system, the ratio of EtOAc from 0% to 85% in 15 min, Flow rate: 70 mL/min; Wave Length: 254 nm. The collected fractions were concentrated under reduced pressure to provide 22-(1-(tert-butoxycarbonyl) piperidin-4-yl)-2,2-dimethyl-4,23-dioxo-3,7,10, 13, 16, 19-hexaoxa-22-azahexacosan-26-oic acid (310 mg, 478 μmol, 19.4%) as a yellow oil. Calc'd for C₃₁H₅₆N₂O₁₂:648.38, found [M+H]: 649.8.

Step 3: Into an 8-mL vial, was placed a mixture of 22-(1-(tert-butoxycarbonyl) piperidin-4-yl)-2,2-dimethyl-4,23-dioxo-3,7,10,13, 16, 19-hexaoxa-22-azahexacosan-26-oic acid (252 mg, 1.50 Eq, 388 μmol), N,N,N′,N′-TetraMethylchloroforMaMidiniuM-hexafluorophosphate (109 mg, 1.50 Eq, 388 μmol), 1-Methylimidazole (N—) (63.8 mg, 61.6 μL, 3.00 Eq, 777 μmol) and DCM (2 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (150 mg, 1 Eq, 259 μmol)_{w} as added and the reaction mixture was stirred at 25° C. for an additional 1 hour. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure. This resulted in tert-butyl 4-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-N-(20,20-dimethyl-18-oxo-3,6,9,12,15, 19-hexaoxahenicosyl)-4-oxobutanamido) piperidine-1-carboxylate formate (180 mg, 143 μmol, 55.4%) as a yellow oil. Calc'd for C₆₂H₈₄C₁₂N₆O₁₇:1254.53, found [M+H−FA]⁺: 1209.5.

Step 4: Into an 8-mL vial, was placed a mixture of tert-butyl 4-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-N-(20,20-dimethyl-18-oxo-3,6,9,12, 15, 19-hexaoxahenicosyl)-4-oxobutanamido) piperidine-1-carboxylate (170 mg, 1 Eq, 140 μmol) and DCM (2 mL), to which was added TFA (0.2 mL). The reaction mixture was stirred at 25° C. for 3 hours. The mixture was concentrated under reduced pressure. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 23-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-20,23-dioxo-19-(piperidin-4-yl)-4,7,10, 13, 16-pentaoxa-19-azatricosanoic acid (150 mg, 0.11 mmol, 81%, 80% Purity) as a white solid. [M+H]=1053.3. Calc'd for C₅₂H₆₆C₁₂N₆O₁₃:1052.41, found [M+H]: 1053.3.

Step 5: Into an 8-mL vial, was placed a mixture of 23-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-20,23-dioxo-19-(piperidin-4-yl)-4,7,10, 13,16-pentaoxa-19-azatricosanoic acid (150 mg, 1 Eq, 142 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (214 mg, 3.00 Eq, 427 μmol), DIEA (91.8 mg, 5.00 Eq, 712 μmol) and DMF (2 mL). The reaction mixture was stirred at 25° C. for 1 hour. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide 2,2′,2″-(10-(2-(4-(N-(17-carboxy-3,6,9,12,15-pentaoxaheptadecyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid--formic acid (1/1) (66.1 mg, 44.5 μmol, 31.2%) as a white solid. Calc'd for C₆₉H₉₄C₁₂N₁₀O₂₂:1484.59, found [M+H−FA]: 1439.7.

### Example 46:2,2′,2″-(10-(2-(4-(N-(carboxymethyl)-4-((3-(1-(3,5-dichlorophenyl)-3-(3,3dimethylmorpholine-4-carbonyl)-7-methoxy-1,4-dihydrochromeno|4,3-clpyrazol-8-yl)phenyl)amino)-4-oxobutanamido) piperidin-1-yl)-2-oxoethyl)-1,4,7,10-tetraazacyclododecane-1,4,7-trivl)triacetic acid

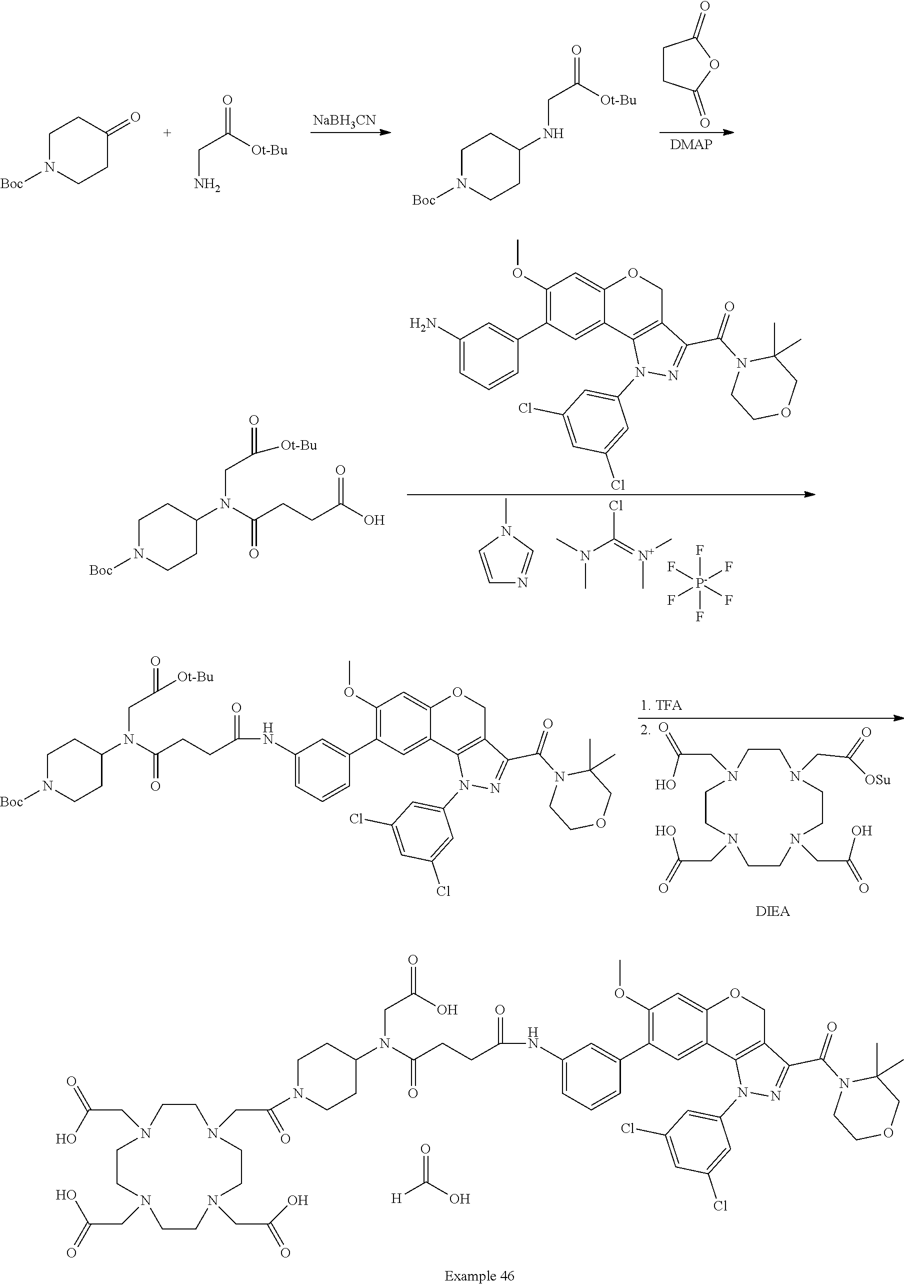

Step 1: Into a 40-mL vial, was placed a mixture of tert-butyl 4-oxopiperidine-1-carboxylate (3.0 g, 2.0 Eq, 15 mmol), tert-butyl glycinate (1.0 g, 1 Eq, 7.6 mmol) and DCE (20 mL). The reaction mixture was stirred at 20° C. for 50 minutes, then sodium cyanoborohydride (1.4 g, 2.9 Eq, 22 mmol)_{w} as added and the reaction mixture was stirred at 25° C. for an additional 4 hours. The collected fractions were concentrated under reduced pressure. The crude product was purified by MPLC with the following conditions: Silica gel column 120 g, PE/EtOAc system, the ratio of EtOAc from 0% to 85% in 15 min, Flow rate: 90 mL/min; Wave Length: 254 nm. The collected fractions were concentrated under reduced pressure to provide tert-butyl 4-((2-(tert-butoxy)-2-oxoethyl)amino) piperidine-1-carboxylate (2.2 g, 5.6 mmol, 73%, 80% Purity) as a yellow oil. Calc'd for C₁₆H₃₀N₂O₄:314.22, found [M+H]⁺: 315.4.

Step 2: Into a 40-mL vial, was placed a mixture of tert-butyl 4-((2-(tert-butoxy)-2-oxoethyl)amino) piperidine-1-carboxylate (1.0 g, 1 Eq, 3.2 mmol), dihydrofuran-2,5-dione (0.64 g, 2.0 Eq, 6.4 mmol), DMAP (0.78 g, 2.0 Eq, 6.4 mmol) and DCE (10 mL). The reaction mixture was stirred at 60° C. for 2 hours. The mixture was diluted with 50 mL of water, extracted with EtOAc (100 mL×3), the combined organic layers were washed with water (100 mL×2), brine (100 mL), dried over anhydrous Na₂SO₄ and concentrated under reduced pressure. The crude product was purified by MPLC with the following conditions: Silica gel column 80 g, PE/EtOAc system, the ratio of EtOAc from 0% to 85% in 15 min, Flow rate: 70 mL/min; Wave Length: 254 nm. The collected fractions were concentrated under reduced pressure to provide 4-((2-(tert-butoxy)-2-oxoethyl) (1-(tert-butoxycarbonyl) piperidin-4-yl)amino)-4-oxobutanoic acid (550 mg, 1.33 mmol, 42%) as a yellow oil. Calc'd for C₂₀H₃₄N₂O₇:414.24, found [M+H]: 415.5.

Step 3: Into an 8-mL vial, was placed a mixture of 4-((2-(tert-butoxy)-2-oxoethyl) (1-(tert-butoxycarbonyl) piperidin-4-yl)amino)-4-oxobutanoic acid (129 mg, 1.20 Eq, 311 μmol), 1-Methylimidazole (N—) (63.8 mg, 61.6 μL, 3.00 Eq, 777 μmol), chloro-N,N,N′,N′-tetramethylformamidinium hexafluorophosphate (109 mg, 1.50 Eq, 388 μmol) and DCM (2 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (150 mg, 1 Eq, 259 μmol)_{w} as added and the reaction mixture was stirred at 25° C. for an additional 1 hour. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide tert-butyl 4-(N-(2-(tert-butoxy)-2-oxoethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidine-1-carboxylate (135 mg, 138 μmol, 53.4%) as a yellow oil. Calc'd for C₅₀H₆₀C₁₂N₆O₁₀: 974.37, found [M+H]: 975.3, 977.3.

Step 4: Into an 8-mL vial, was placed a mixture of tert-butyl 4-(N-(2-(tert-butoxy)-2-oxoethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidine-1-carboxylate (125 mg, 1 Eq, 128 μmol) and DCM (2 mL), to which was added TFA (0.2 mL). The reaction mixture was stirred at 25° C. for 3 hours. The mixture was concentrated under reduced pressure. The crude product was purified by Prep-HPLC with the following conditions: Column: SunFire prep OBD 19*150 mm 5 μm; Mobile Phase A: Water (0.05% TFA); Mobile

Phase B: ACN; Gradient: 25% B to 65% B in 8 min; Flow rate: 20 mL/min; Wave Length: 220 nm. The collected fractions were dried by lyophilization to provide N-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoyl)-N-(piperidin-4-yl)glycine (120 mg, 0.12 mmol, 91%, 80% Purity) as a white solid. Calc'd for C₄₁H₄₄C₁₂N₆O8:818.26, found [M+H]: 819.2, 821.2.

Step 5: Into an 8-mL vial, was placed a mixture of N-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoyl)-N-(piperidin-4-yl)glycine (120 mg, 1 Eq, 146 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (220 mg, 3.00 Eq, 439 μmol), DIEA (94.6 mg, 127 μL, 5.00 Eq, 732 μmol) and DMF (2 mL). The reaction mixture was stirred at 25° C. for 1 hour. The crude product was purified by Prep-HPLC with the following conditions: Column: SunFire prep OBD 19*150 mm 5 μm; Mobile Phase A: Water (0.05% FA); Mobile Phase B: ACN; Gradient: 20% B to 60% B in 8 min; Flow rate: 20 mL/min; Wave Length: 220 nm. The collected fractions were dried by lyophilization to provide 2,2′,2″-(10-(2-(4-(N-(carboxymethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid--formic acid (1/1) (28.5 mg, 22.8 μmol, 15.5%) as a white solid. Calc'd for C₅₈H₇₂C₁₂N₁₀O₁₇:1250.45, found [M+H−FA]⁺: 1205.6, 1207.6.

### Example 47:2,2′,2″-(10-(2-((1,3-bis(2-carboxyethoxy)-2-((3-((3-(1-(3,5-dichlorophenyl)-3(3,3-dimethylmorpholine-4-carbonyl)-7-methoxy-1,4-dihydrochromeno|4,3-clpyrazol-8-yl)phenyl)amino)-3-oxopropoxy)methyl) propan-2-yl)amino)-2-oxoethyl)-1,4,7,10-tetraazacyclododecane-1,4,7-triyl)triacetic acid

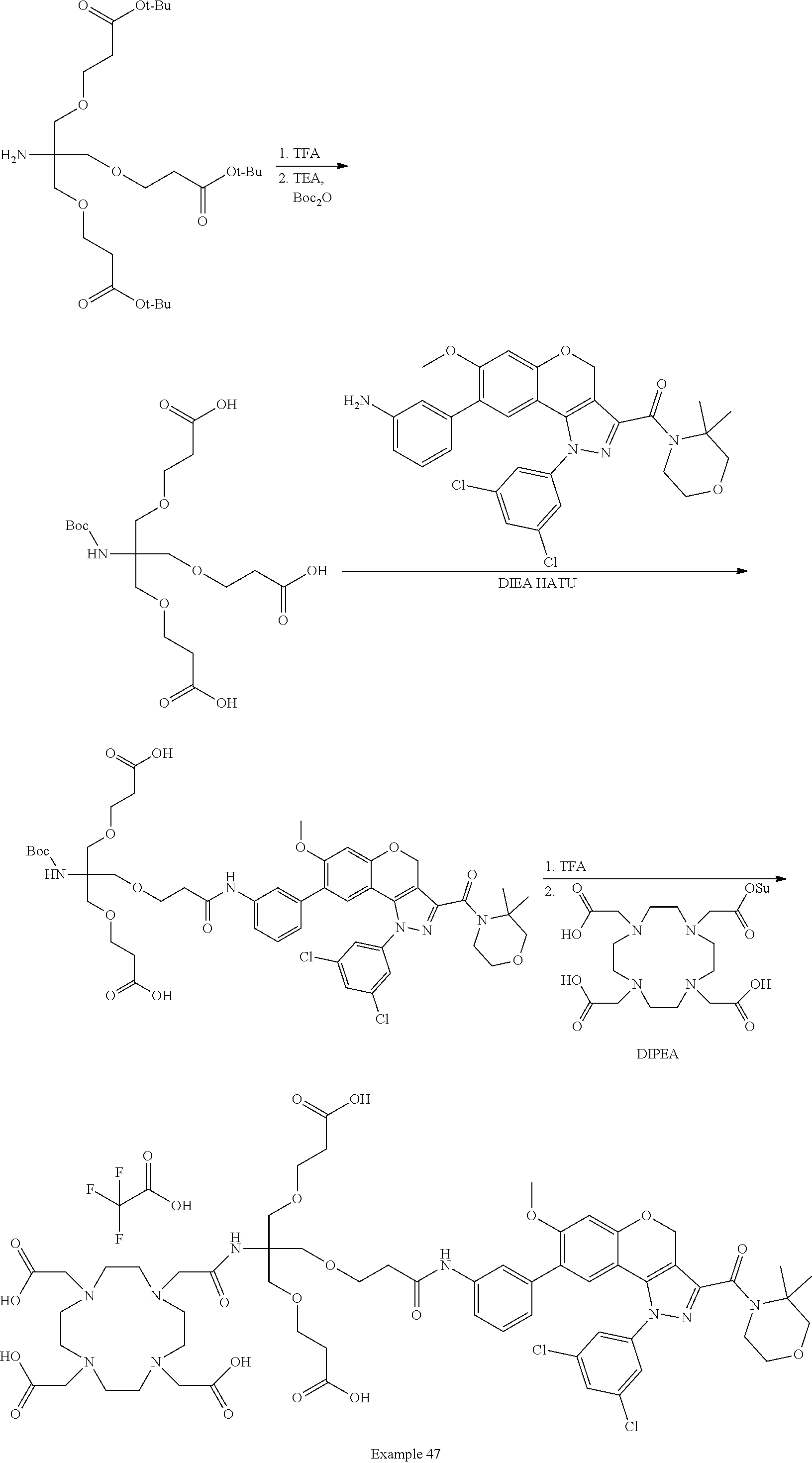

Step 1: Into a 50-mL round-bottom flask, was placed a mixture of di-tert-butyl 3,3′-((2-amino-2-((3-(tert-butoxy)-3-oxopropoxy)methyl) propane-1,3-diyl)bis(oxy))dipropionate (600 mg, 1 Eq, 1.19 mmol) to which was added DCM (12 mL) and TFA (4 mL). The reaction mixture was stirred at 25° C. for 1 hour. The mixture was concentrated under reduced pressure to provide 3,3′-((2-amino-2-((2-carboxyethoxy)methyl) propane-1,3-diyl)bis(oxy))dipropionic acid (500 mg, 1.3 mmol, 110%, 90% Purity) as a colorless oil. Calc'd for C₁₃H₂₃NO₉:337.13, found [M+H]⁺: 338.0.

Step 2: Into a 50-mL round-bottom flask, was placed a mixture of 3,3′-((2-amino-2-((2-carboxyethoxy)methyl) propane-1,3-diyl)bis(oxy))dipropionic acid (500 mg, 1 Eq, 1.48 mmol) in MeCN (5 mL), added TEA (750 mg, 1.03 mL, 5.00 Eq, 7.41 mmol) and BoczO(485 mg, 511 μL, 1.50 Eq, 2.22 mmol). The mixture was stirred at 25° C. for 2 hours. The mixture was directly purified by MPLC with the following conditions: Column, WelFlash™, C18 120 g, Spherical 20-40 μm; Mobile phase, Water (0.05% TFA) and ACN (5% ACN to 5% ACN in 1 min, 5% ACN up to 98% in 6 min, 98% ACN to 98% in 3 min); Total flow rate, 70 mL/min; Detector, ELSD. The collected fractions were dried by lyophilization to provide 3,3′-((2-((tert-butoxycarbonyl)amino)-2-((2-carboxyethoxy)methyl) propane-1,3-diyl)bis(oxy))dipropionic acid (280 mg, 0.58 mmol, 39%, 90% Purity) as a colorless-oil. Calc'd for C18H₃₁NO_{11:437.19}, found [M+Na]*: 460.1.

Step 3: Into a 40-mL vial, was placed a mixture of 3,3′-((2-((tert-butoxycarbonyl)amino)-2-((2-carboxyethoxy)methyl) propane-1,3-diyl)bis(oxy))dipropionic acid (280 mg, 1 Eq, 640 μmol), in which was added DMF (3 mL), HATU (240 mg, 0.986 Eq, 631 μmol) and DIEA (248 mg, 334 μL, 3.00 Eq, 1.92 mmol). The mixture was stirred at 25° C. for 10 mins. To the above mixture was added (8-(3-aminophenyl)-1-(3,5-dichlorophenyl)-7-methoxy-1,4-dihydrochromeno[4,3-c]pyrazol-3-yl) (3,3-dimethylmorpholino) methanone (290 mg, 0.782 Eq, 500 μmol). The resulting mixture was stirred 25° C. for 1 hour. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 5% ACN up to 98% in 6 min, 98% ACN to 98% in 3 min); Total flow rate, 70 mL/min; Detector, UV 220 nm. The collected fractions were dried by lyophilization to provide 3,3′-((2-((tert-butoxycarbonyl)amino)-2-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)methyl) propane-1,3-diyl)bis(oxy))dipropionic acid (65 mg, 59 μmol, 9.1%, 90% Purity) as an off-white solid. Calc'd for C₄₈H₅₇C₁₂N₅O₁₄:997.32, found [M+Na]: 1020.2, 1022.2.

Step 4: Into a 50-mL round-bottom flask, was placed a mixture of 3,3′-((2-((tert-butoxycarbonyl)amino)-2-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)methyl) propane-1,3-diyl)bis(oxy))dipropionic acid (65 mg, 1 Eq, 65 μmol), to which was added DCM (5 mL) and TFA (1 mL). The reaction mixture was stirred at 25° C. for 1 hour. The mixture was concentrated under reduced pressure. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide 3,3′-((2-amino-2-((2-carboxyethoxy)methyl) propane-1,3-diyl)bis(oxy))dipropionic acid (500 mg, 1.3 mmol, 110%, 90% Purity) as a colorless oil. Calc'd for C₄₅H₅₀C₁₂F3N₅O₁₄:1011.27, found [M+H−TFA]: 898.3, 900.3.

Step 5: Into an 8-mL vial, was placed a mixture of 3,3′-((2-amino-2-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)methyl) propane-1,3-diyl)bis(oxy))dipropionic acid (45 mg, 1 Eq, 50 μmol) in DMF (1 mL), then DIEA (66 mg, 89 μL, 10 Eq, 0.51 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (75 mg, 3.0 Eq, 0.15 mmol)_{w} as added. The resulting mixture was stirred at 25° C. for 2 hours. The crude product was purified by Prep-HPLC with the following conditions: Column: SunFire prep OBD 30*150 mm 5 μm; Mobile Phase A: Water (0.1% TFA); Mobile Phase B: ACN; Gradient: 10% B to 25% B 25% in 8 min, 25% B to 25% B in 1 min; Flow rate: 60 mL/min; Wave Length: 220 nm to afford two isomers. The front peak fractions were dried by lyophilization to afford 2,2′,2″-(10-(2-((1,3-bis(2-carboxyethoxy)-2-((3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)methyl) propan-2-yl)amino)-2-oxoethyl)-1,4,7,10-tetraazacyclododecane-1,4,7-triyl)triacetic acid--2,2,2-trifluoroacetic acid (1/1) (5.1 mg, 3.3 μmol, 6.6%, 90% Purity) as an off-white solid. Calc'd for C₆₁H₇₆C₁₂F3N₉O₂₁:1397. 45, found [M+H−TFA]⁺: 1284.6, 1286.6.

### Example 48:2,2′,2″-(10-(2-((3-carboxy-5-(4-((3-(1-(3,5-dichlorophenyl)-3-(3,3dimethylmorpholine-4-carbonyl)-7-methoxy-1,4-dihydrochromeno|4,3-c]pyrazol-8-yl)phenyl)amino)-4-oxobutanamido)phenyl)amino)-2-oxoethyl)-1,4,7,10-tetraazacyclododecane-1,4,7-triyl)triacetic acid

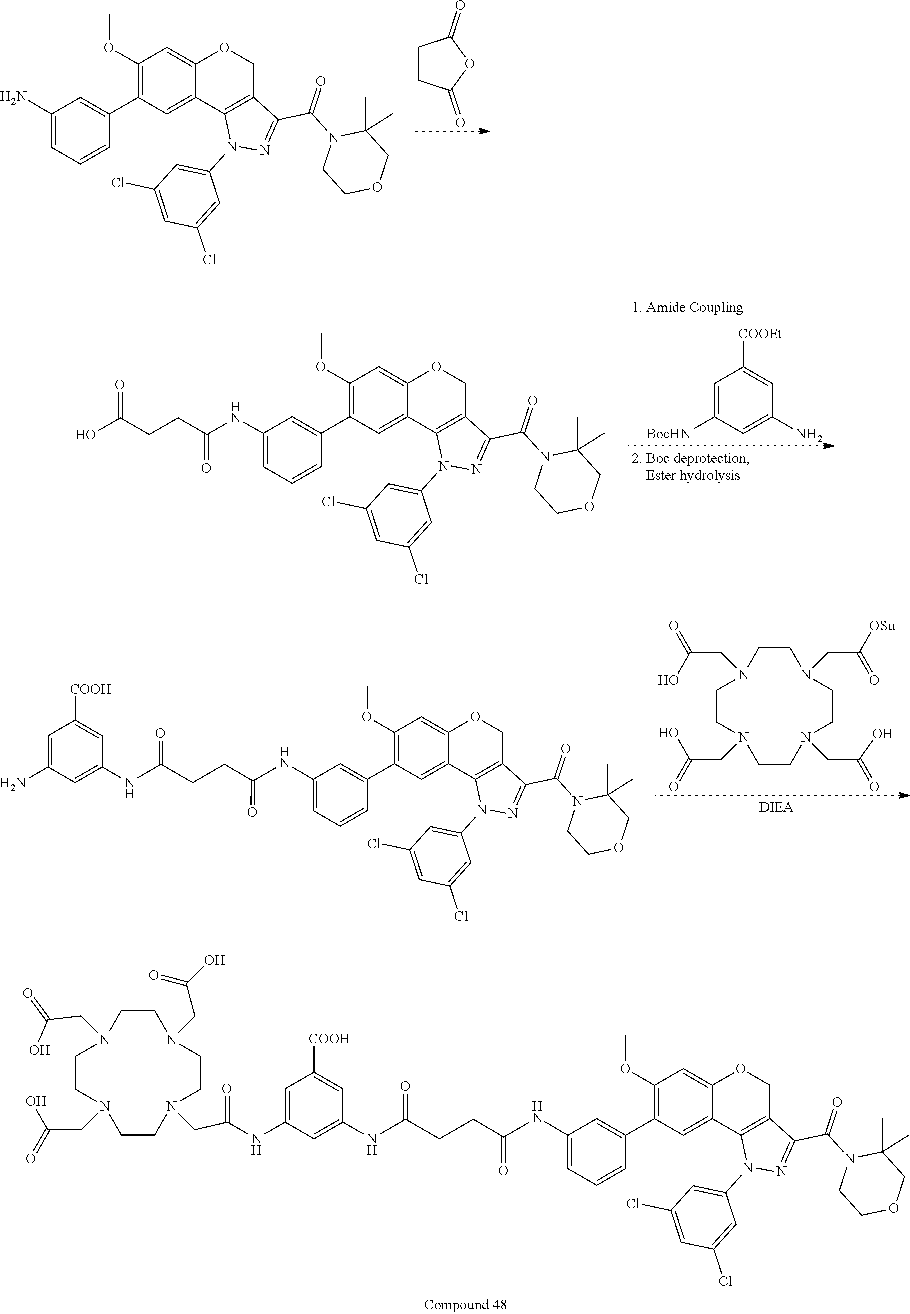

Compound 48 may be synthesized according to the preceding scheme.

### Example 49: Indium Complex of Compound 1 (Compound 1A)

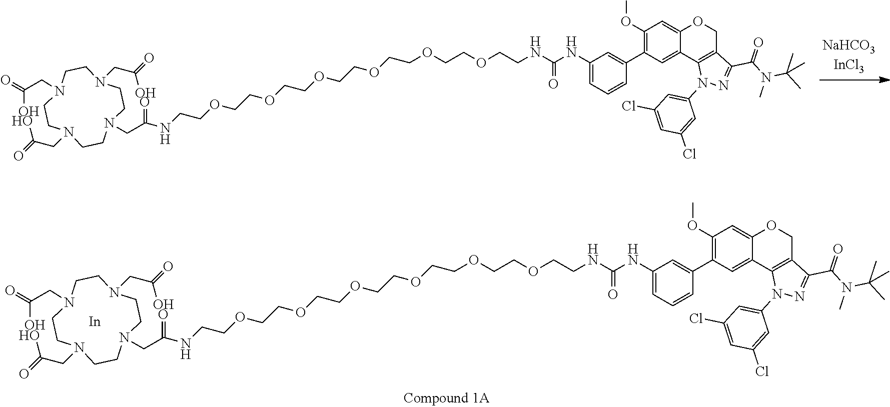

Into a 8 mL flask were added a mixture of 2,2′,2″-(10-(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8,11, 14, 17,20-hexaoxa-2,23-diazapentacosan-25-yl)-1,4,7,10-tetraazacyclododecane-1,4,7-triyl)triacetic acid (Example 1, 40 mg, 1 Eq, 31 μmol), sodium bicarbonate (20 mg, 9.3 μL, 7.7 Eq, 0.24 mmol), indium trichloride (25 mg, 7.2 uL, 3.6 Eq, 0.11 mmol), ACN (0.4 mL), and water (0.2 mL). The mixture was stirred for 1 hour at 80° C. The mixture was diluted with DMSO(4 mL), filtered and the filtrate was purified by Prep-HPLC with the following conditions: Column, SunFire Prep C18 OBD Column, 19*150 mm 5 μm 10 nm; mobile phase, Water (0.05% FA) and ACN (30% ACN up to 75% in 15 min); Total flow rate, 20 mL/min; Detector, UV 220 nm. Purification provided indium (III) 2,2′,2″-(10-(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8,11, 14, 17,20-hexaoxa-2,23-diazapentacosan-25-yl)-1,4,7,10-tetraazacyclododecane-1,4,7-triyl)triacetate (18.9 mg, 13.5 μmol, 43%) as an off-white solid. Calc'd for C₆₀H₈₁C₁₂InN10017:1398.4, found [M+H]⁺: 1399.7, 1401.9.

### Example 50: Indium Complex of Compound 2 (Compound 2A)

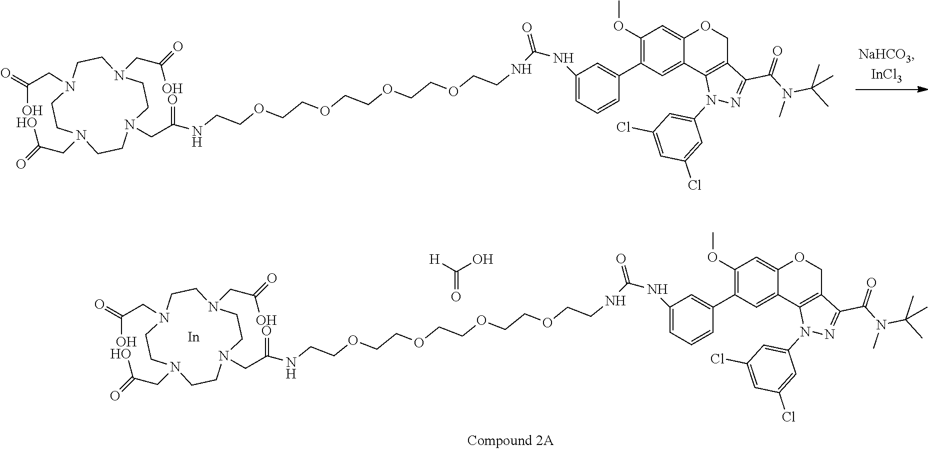

Into an 8 mL flask were added a mixture of 2,2′,2″-(10-(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1,18-dioxo-5,8, 11, 14-tetraoxa-2, 17-diazanonadecan-19-yl)-1,4,7,10-tetraazacyclododecane-1,4,7-triyl)triacetic acid (from Example 2, 10 mg, 1 Eq, 8.3 μmol), sodium bicarbonate (6 mg, 3 μL, 9 Eq, 0.07 mmol), indium trichloride (6 mg, 2 μL, 3 Eq, 0.03 mmol), ACN (0.2 mL) and water (0.1 mL). The mixture was stirred for 2 hours at 80° C. The mixture was diluted with DMSO(4 mL), filtered and the filtrate was purified by Prep-HPLC using the following conditions: Column, SunFire Prep C18 OBD Column, 19*150 mm 5 μm 10 nm; mobile phase, Water (0.05% FA) and ACN (30% ACN up to 80% in 15 min); Total flow rate, 20 mL/min; Detector, UV 220 nm. Purification provided indium (III) 2,2′,2″-(10-(1-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-1,18-dioxo-5,8,11,14-tetraoxa-2,17-diazanonadecan-19-yl)-1,4,7,10-tetraazacyclododecane-1,4,7-triyl)triacetate 1FA salt (6.5 mg, 4.8 μmol, 57%) as a white solid. Calc'd for C₅₇H₇₅Cl2InN10017:1356.4, found [M+H−FA]⁺: 1311.1, 1312.8.

### Example 51: Indium Complex of Compound 3 (Compound 3A)

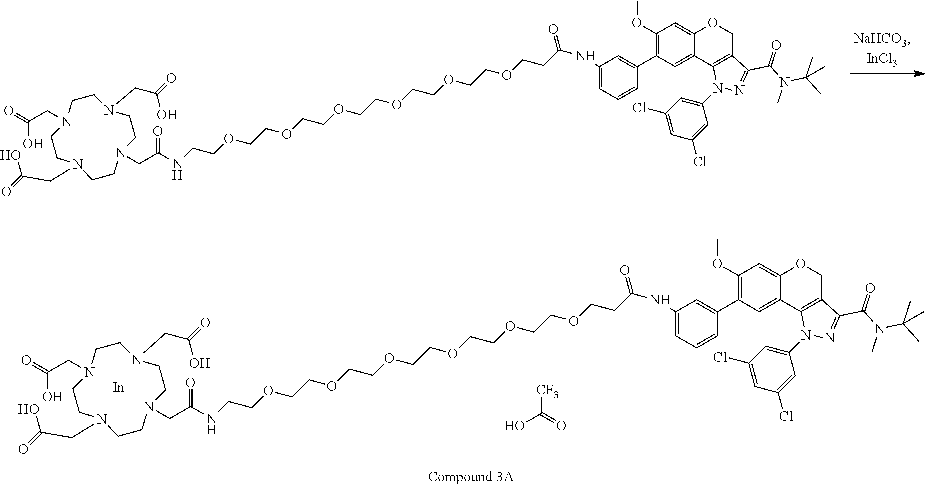

Into an 8 mL flask were added a mixture of 2,2′,2″-(10-(24-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-2,24-dioxo-6,9,12, 15,18,21-hexaoxa-3-azatetracosyl)-1,4,7,10-tetraazacyclododecane-1,4,7-triyl)triacetic acid (from Example 3, 6 mg, 1 Eq, 5 μmol), sodium bicarbonate (4 mg, 2 uL, le+1 Eq, 0.05 mmol), indium trichloride (4 mg, 1 μL, 4 Eq, 0.02 mmol), ACN (0.2 mL), and water (0.1 mL). The mixture was stirred for 2 hours at 80° C. The mixture was diluted with 4 mL of DMSO(4 mL), filtered and the filtrate was purified by Prep-HPLC with the following conditions: Column, SunFire Prep C18 OBD Column, 19*150 mm 5 μm 10 nm; mobile phase, Water (0.05% TFA) and ACN (30% ACN up to 75% in 15 min); Total flow rate, 20 mL/min; Detector, UV 220 nm. Purification provided indium (III) 2,2′,2″-(10-(24-((3-(3-(tert-butyl(methyl) carbamoyl)-1-(3,5-dichlorophenyl)-7-methoxy-1,4-dihydrochromeno[4,3-c]pyrazol-8-yl)phenyl)amino)-2,24-dioxo-6,9,12,15,18,21-hexaoxa-3-azatetracosyl)-1,4,7,10-tetraazacyclododecane-1,4,7-triyl)triacetate 1TFA salt (3.3 mg, 2.2 μmol, 50%) as a white solid. Calc'd for C₆₂H₈₁C₁₂F3InN9O19:1497.4, found [M+H−TFA]⁺: 1384.3, 1386.3.

### Example 52: Indium Complex of Compound 5 (Compound 5A)

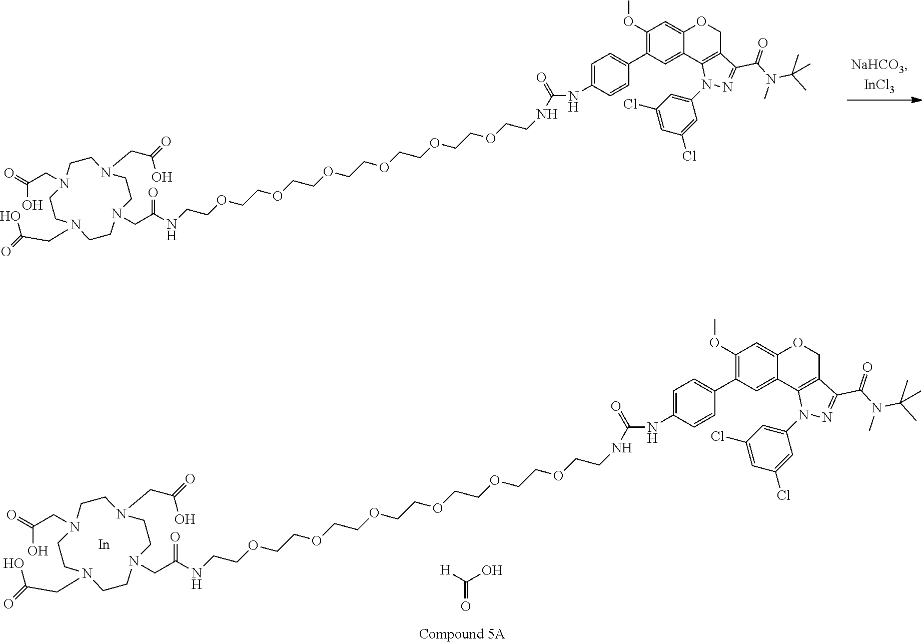

Into an 8 mL flask were added a mixture of 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8,11,14, 17,20-hexaoxa-2,23-diazapentacosan-25-yl)-1,4,7,10-tetraazacyclododecane-1,4,7-triyl)triacetic acid (from Example 5, 10 mg, 1 Eq, 7.8 μmol), sodium bicarbonate (6 mg, 3 μL, 9 Eq, 0.07 mmol), indium trichloride (7 mg, 2 μL, 4 Eq, 0.03 mmol), ACN (0.2 mL) and water (0.1 mL). The mixture was stirred for 2 hours at 80° C. The mixture was diluted with DMSO(4 mL), filtered and the filtrate was purified by Prep-HPLC with the following conditions: Column, SunFire Prep C18 OBD Column, 19*150 mm 5 μm 10 nm; mobile phase, Water (0.05% FA) and ACN (30% ACN up to 75% in 15 min); Total flow rate, 20 mL/min; Detector, UV 220 nm. Purification provided indium (III) 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,24-dioxo-5,8,11,14,17,20-hexaoxa-2,23-diazapentacosan-25-yl)-1,4,7,10-tetraazacyclododecane-1,4,7-triyl)triacetate 1FA salt (8.1 mg, 5.6 μmol, 72%) as a white solid. Calc'd for C₆₁H₈₃C₁₂InN10019:1444.4, found [M+H−FA]⁺: 1399.6, 1400.7.

### Example 53: Indium Complex of Compound 6 (Compound 6A)

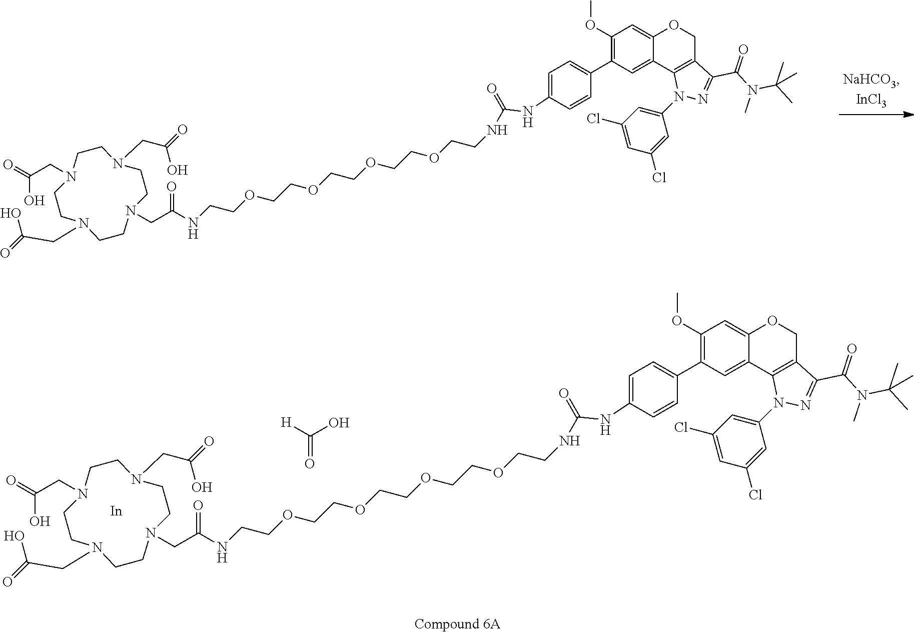

Into an 8 mL flask were added a mixture of 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,18-dioxo-5,8,11, 14-tetraoxa-2,17-diazanonadecan-19-yl)-1,4,7,10-tetraazacyclododecane-1,4,7-triyl)triacetic acid (from Example 6, 15 mg, 1 Eq, 12 μmol), sodium bicarbonate (10 mg, 4.6 μL, 9.5 Eq, 0.12 mmol), indium trichloride (15 mg, 4.3 uL, 5.4 Eq, 68 μmol), ACN (0.2 mL) and water (0.1 mL). The mixture was stirred for 2 hours at 80° C. The mixture was diluted with DMSO(4 mL), filtered and the filtrate was purified by Prep-HPLC with the following conditions: Column, SunFire Prep C18 OBD Column, 19*150 mm 5 μm 10 nm; mobile phase, Water (0.05% FA) and ACN (30% ACN up to 75% in 15 min); Total flow rate, 20 mL/min; Detector, UV 220 nm. Purification provided indium (III) 2,2′,2″-(10-(1-((4-(3-(tert-butyl(methyl) carbamoyl)-1-(3,5-dichlorophenyl)-7-methoxy-1,4-dihydrochromeno[4,3-c]pyrazol-8-yl)phenyl)amino)-1,18-dioxo-5,8,11, 14-tetraoxa-2, 17-diazanonadecan-19-yl)-1,4,7,10-tetraazacyclododecane-1,4,7-triyl)triacetate 1FA salt (6.5 mg, 4.8 μmol, 38%) as a white solid. Calc'd for C₅₇H₇₅Cl2InN10017:1356.4, found [M+H−FA]⁺: 1311.5, 1313.5.

### Example 54: Indium Complex of Compound 20 (Compound 20A)

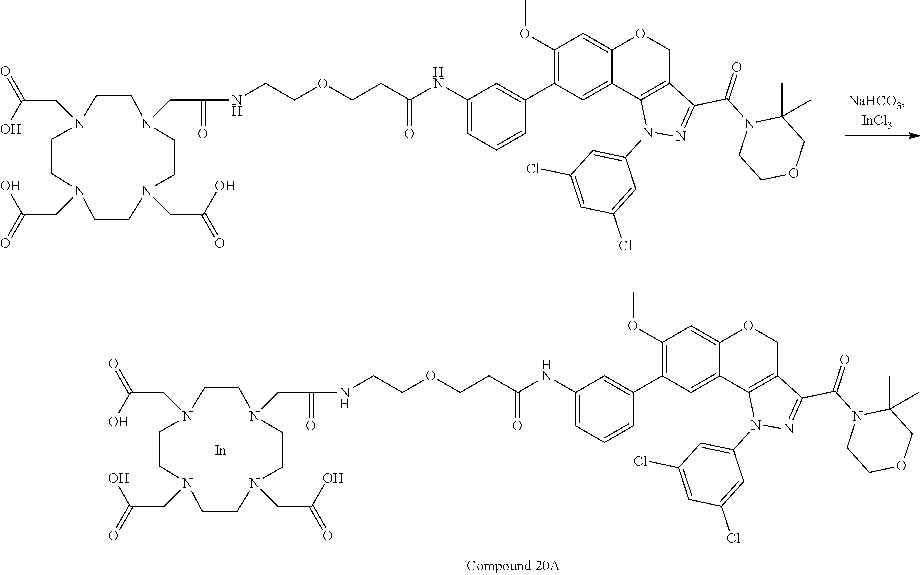

Into an 8-mL vial, was placed a mixture of 2,2′,2″-(10-(2-((2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethyl)amino)-2-oxoethyl)-1,4,7,10-tetraazacyclododecane-1,4,7-triyl)triacetic acid (from Example 20, 20 mg, 1 Eq, 19 μmol), sodium bicarbonate (10 mg, 4.6 μL, 6.4 Eq, 0.12 mmol), indium (III) chloride (10 mg, 2.9 Eq, 54 μmol), ACN (1.0 mL) and H₂O(0.5 mL). The reaction mixture was stirred at 80° C. for 2 hours. The crude product was purified by Prep-HPLC with the following conditions:

Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide Compound 20A (10.9 mg, 8.80 μmol, 48%) as an off-white solid. Calc'd for C₅₂H₆₂Cl2InN9Ois: 1237.3, found [M+H−FA]⁺: 1192.4.

### Example 55: Indium Complex of Compound 19 (Compound 19A)

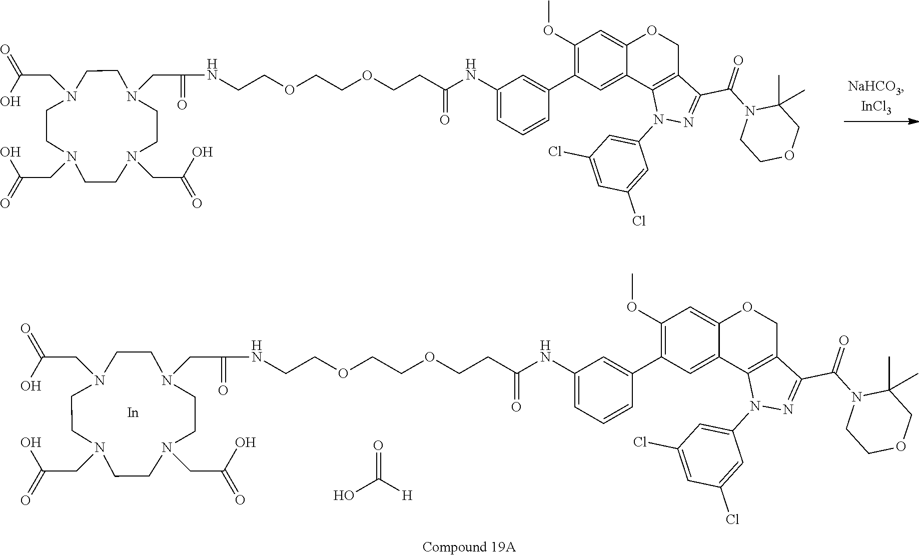

Into an 8-mL round bottom flask, was placed a mixture of 2,2′,2″-(10-(2-((2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl)amino)-2-oxoethyl)-1,4,7,10-tetraazacyclododecane-1,4,7-triyl)triacetic acid (from Example 19, 20 mg, 1 Eq, 18 μmol), sodium bicarbonate (9.0 mg, 4.2 μL, 6.0 Eq, 0.11 mmol), indium (III) chloride (10.1 mg, 3.1 Eq, 54.4 μmol) ACN (1.0 mL), and H₂O(0.5 mL). The reaction mixture was stirred at 80° C. for 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide Compound 19A (6.8 mg, 5.3 μmol, 30%) as an off white solid. Calc'd for Cs4H₆₆Cl2InN9016:1281.3, found [M+H−FA]⁺: 1236.5.

### Example 56: Indium Complex of Compound 18 (Compound 18A)

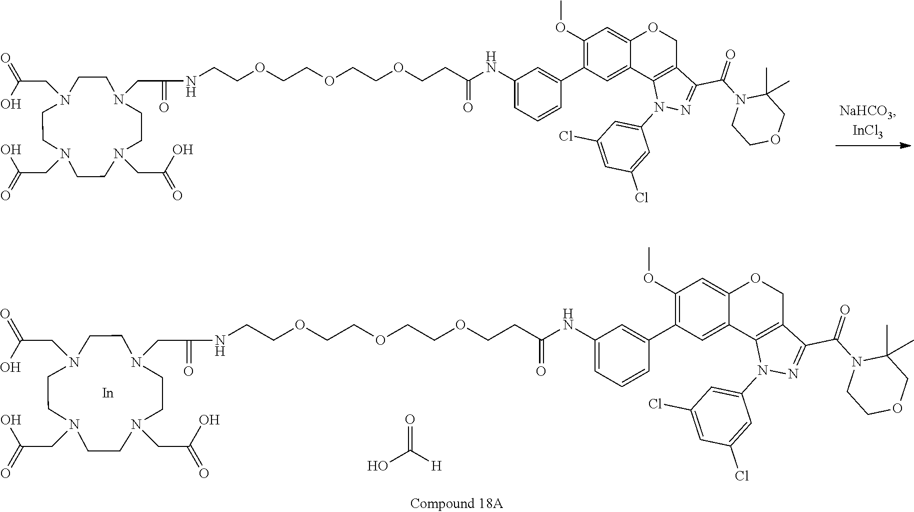

Into a 8-mL vial, was placed a mixture of 2,2′,2″-(10-(15-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,15-dioxo-6,9,12-trioxa-3-azapentadecyl)-1,4,7,10-tetraazacyclododecane-1,4,7-triyl)triacetic acid (from Example 18, 20 mg, 1 Eq, 17 μmol), indium (III) chloride (10 mg, 3.1 Eq, 54 μmol), sodium bicarbonate (9.0 mg, 4.2 μL, 6.3 Eq, 0.11 mmol), ACN (1.0 mL) and H₂O(0.5 mL). The reaction mixture was stirred at 80° C. for 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide the Compound 18A (9.8 mg, 7.4 μmol, 43%) as an off-white solid. Calc'd for C₅₆H₇₀Cl2InN9017:1325.3, found [M+H−FA]⁺: 1280.5.

### Example 57: Lutetium Complex of Compound 17 (Compound 17B)

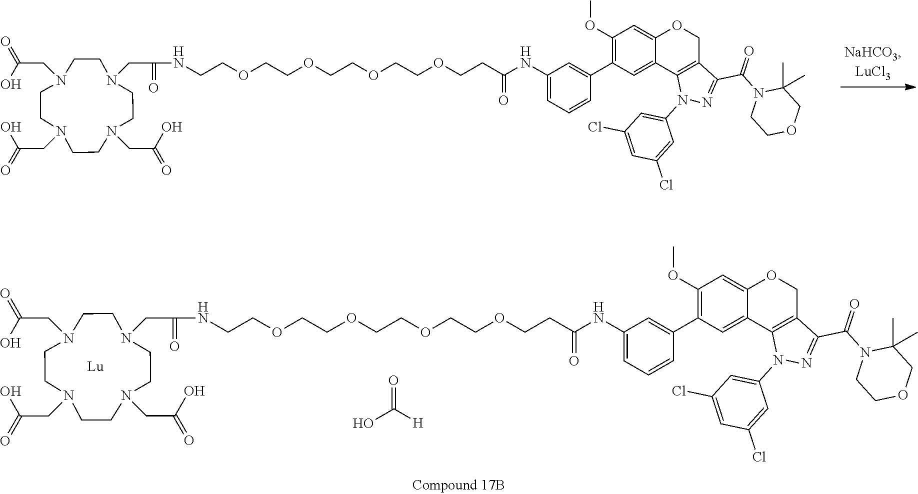

Into an 8-mL vial, was placed a mixture of 2,2′,2″-(10-(18-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,18-dioxo-6,9,12,15-tetraoxa-3-azaoctadecyl)-1,4,7,10-tetraazacyclododecane-1,4,7-triyl)triacetic acid (from Example 17, 27.2 mg, 1 Eq, 22.4 μmol), lutetium (III) chloride (19 mg, 4.8 uL, 3.0 Eq, 68 μmol), sodium bicarbonate (11.3 mg, 5.23 uL, 6.0 Eq, 135 μmol), H₂O(0.5 mL), and ACN (1.0 mL). The reaction mixture was stirred at 80° C. for 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide Compound 17B (16.9 mg, 11.8 μmol, 52.7%) as an off-white solid. Calc'd for C₅₈H₇₄Cl2LuN9018:1429.4, found [M+H−FA]*: 1384.6.

### Example 58: Indium Complex of Compound 16 (Compound 16A)

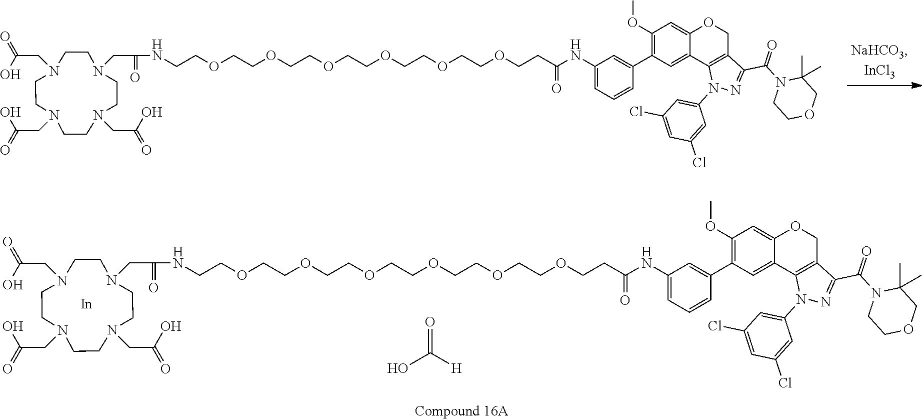

Into an 8-mL vial, was placed a mixture of 2,2′,2″-(10-(24-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,24-dioxo-6,9,12, 15,18,21-hexaoxa-3-azatetracosyl)-1,4,7,10-tetraazacyclododecane-1,4,7-triyl)triacetic acid (from Example 16, 20.0 mg, 1 Eq, 15.4 μmol), indium (III) chloride (9.0 mg, 3.2 Eq, 48 μmol), sodium bicarbonate (8.5 mg, 3.9 μL, 6.6 Eq, 0.10 mmol), H₂O(0.5 mL), and ACN (1.0 mL). The reaction mixture was stirred at 80° C. for 2 hours. The crude product was purified by Prep-HPLC with the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide Compound 16A (9.7 mg, 6.6 μmol, 43%) as an off-white solid. Calc'd for C₆₂H₈₂C₁₂InN9020:1457.4, found [M+H−FA]⁺: 1412.2.

### Example 59: Indium Complex of Compound 8 (Compound 8A)

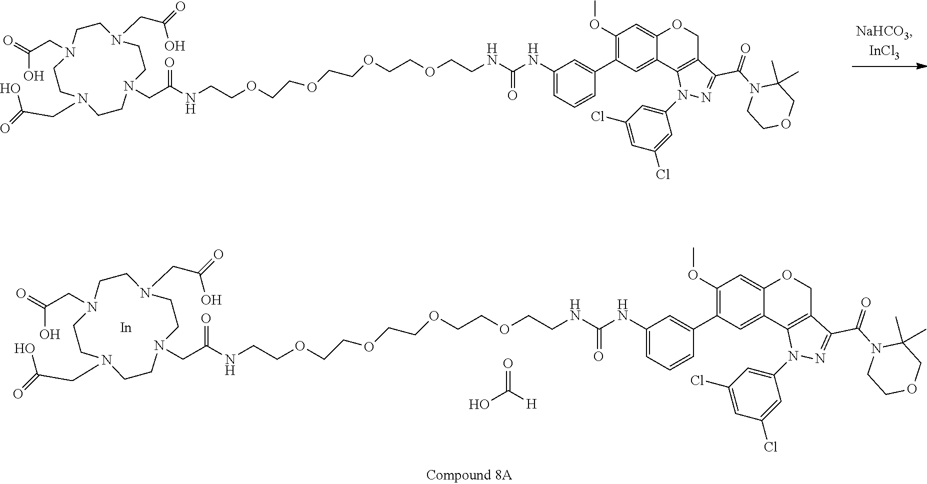

Into an 8-mL vial, was placed a mixture of 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,18-dioxo-5,8,11,14-tetraoxa-2,17-diazanonadecan-19-yl)-1,4,7,10-tetraazacyclododecane-1,4,7-triyl)triacetic acid (from Example 8, 20 mg, 1 Eq, 16 μmol), indium (III) chloride (10.2 mg, 3.4 Eq, 54.9 μmol), sodium bicarbonate (9.6 mg, 4.4 uL, 7.0 Eq, 0.11 mmol), H₂O(0.5 mL) and ACN (1.0 mL). The reaction mixture was stirred at 80° C. for 2 hours. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide Compound 8A (9.6 mg, 6.9 μmol, 43%) as an off-white solid. Calc'd for C₅₈H₇₅C₁₂InN10018:1384.4, found [M+H−FA]⁺: 1339.5.

### Example 60: Indium Complex of Compound 10 (Compound 10A)

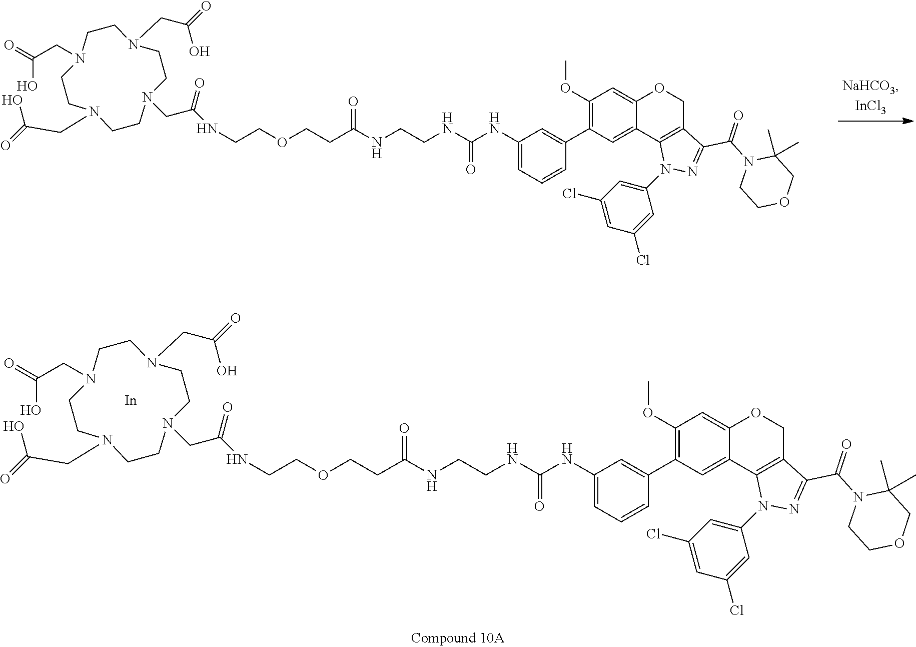

Into an 8 mL flask were added a mixture of 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,6,13-trioxo-9-oxa-2,5,12-triazatetradecan-14-yl)-1,4,7,10-tetraazacyclododecane-1,4,7-triyl)triacetic acid (from Example 10, 12.5 mg, 1 Eq, 10.7 μmol), indium trichloride (10 mg, 2.9 μL, 4.2 Eq, 45 μmol), sodium bicarbonate (5 mg, 2 μL, 6 Eq, 0.06 mmol), ACN (0.2 mL), and water (0.1 mL). The mixture was stirred for 2 hours at 80° C. The mixture was diluted with DMSO(4 mL), filtered and the filtrate was purified by Prep-HPLC with the following conditions: Column, SunFire Prep C18 OBD Column, 19*150 mm 5 μm 10 nm; mobile phase, Water (0.05% FA) and ACN (30% ACN up to 75% in 15 min); Total flow rate, 20 mL/min; Detector, UV 220 nm. Purification provided indium (III) 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-1,6, 13-trioxo-9-oxa-2,5, 12-triazatetradecan-14-yl)-1,4,7,10-tetraazacyclododecane-1,4,7-triyl)triacetate (8.2 mg, 6.4 μmol, 60%) as a white solid. Calc'd for C₅₄H₆₆C₁₂InN1014:1277.3, found [M+H]⁺: 1278.7, 1280.7.

### Example 61: Indium Complex of Compound 11 (Compound 11A)

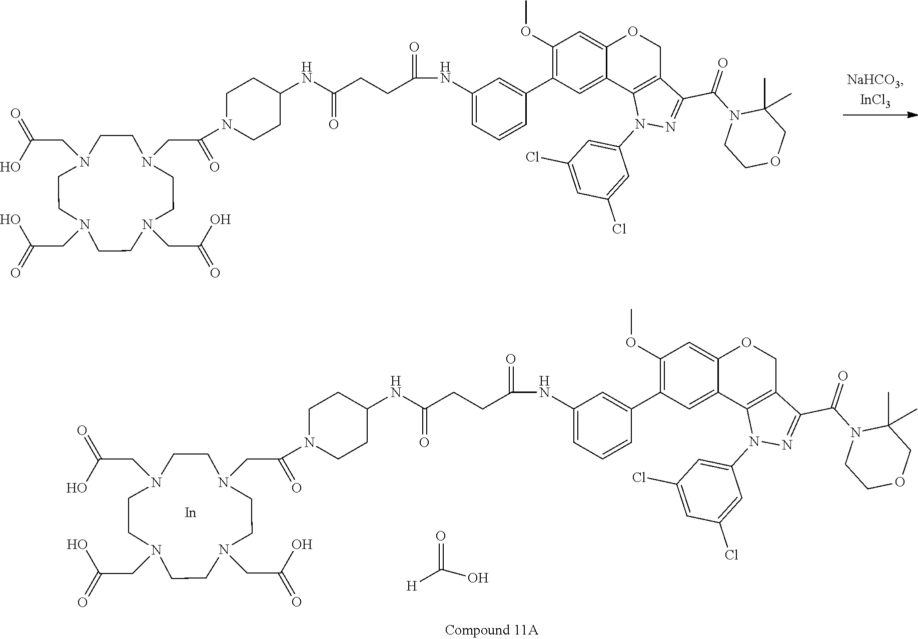

Into an 8-mL vial, was placed a mixture of 2,2′,2″-(10-(2-(4-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid (from Example 11, 19 mg, 1 Eq, 17 μmol), sodium bicarbonate (7.0 mg, 3.2 μL, 5.0 Eq, 83 μmol), indium (III) chloride (12 mg, 3.9 Eq, 65 μmol), MeCN (0.6 mL) and H₂O(0.3 mL). The reaction mixture was stirred at 80° C. for 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were dried by lyophilization to provide Compound 11A (11.2 mg, 8.58 μmol, 52%) as a white solid. Calc'd for C₅₆H₆₇C₁₂InN10015:1304.3, found [M+H−FA]⁺: 1259.6.

### Example 62: Indium Complex of Compound 14 (Compound 14A)

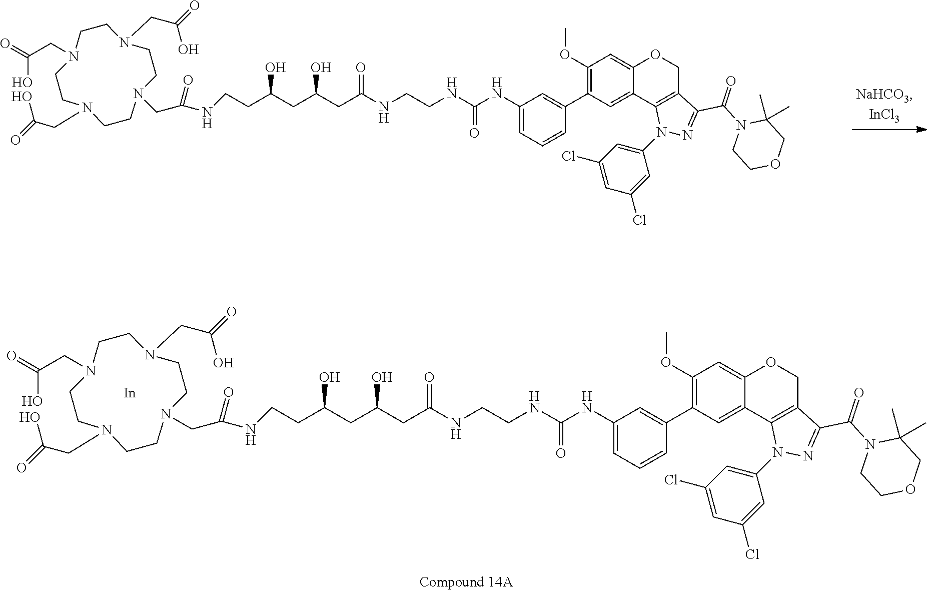

Into an 8 mL flask were added a mixture of 2,2′,2″-(10-(2-(((3R,5R)-7-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,5-dihydroxy-7-oxoheptyl)amino)-2-oxoethyl)-1,4,7,10-tetraazacyclododecane-1,4,7-triyl)triacetic acid (from Example 14, 17 mg, 1 Eq, 14 μmol), indium trichloride (16 mg, 4.6 μL, 5.2 Eq, 72 μmol), sodium bicarbonate (9 mg, 4 μL, 8 Eq, 0.1 mmol), ACN (0.2 mL) and water (0.1 mL). The mixture was stirred for 2 hours at 80° C. The mixture was diluted with DMSO(4 mL), filtered and the filtrate was purified by Prep-HPLC with the following conditions: Column, SunFire Prep C18 OBD Column, 19*150 mm 5 μm 10 nm; mobile phase, Water (0.05% FA) and ACN (30% ACN up to 75% in 15 min); Total flow rate, 20 mL/min; Detector, UV 220 nm. Purification provided indium (III) 2,2′,2″-(10-(2-(((3R,5R)-7-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,5-dihydroxy-7-oxoheptyl)amino)-2-oxoethyl)-1,4,7,10-tetraazacyclododecane-1,4,7-triyl)triacetate (5.5 mg, 4.2 μmol, 30%) as a white solid. Calc'd for C₅₆H₇C₁₂InNnOis: 1321.3, found [M+H]⁺: 1322.7, 1324.7.

### Example 63: Indium Complex of Compound 15 (Compound 15A)

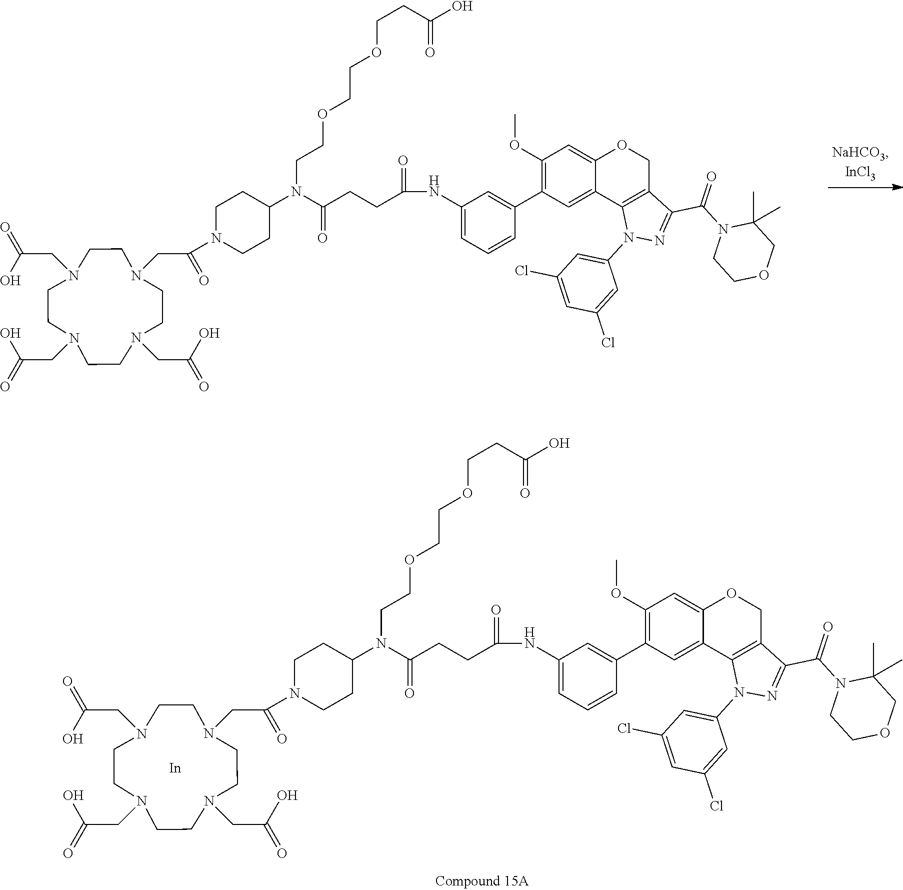

Into an 8-mL vial, was placed a mixture of 2,2′,2″-(10-(2-(4-(N-(2-(2-(2-carboxyethoxy)ethoxy)ethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetic acid (from Example 15, 18.5 mg, 1 Eq, 14.1 μmol), sodium bicarbonate (5.95 mg, 2.75 uL, 5.01 Eq, 70.8 μmol), indium (III) chloride (10.5 mg, 4.00 Eq, 56.5 μmol), MeCN (0.6 mL) and H₂O (0.3 mL). The reaction mixture was stirred at 80° C. for 2 hours. The mixture was directly purified by MPLC with the following conditions: Column, WelFlash™, C18 120 g, Spherical 20-40 μm; Mobile phase, Water (0.05% NH₃.H₂O) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were dried by lyophilization to provide indium (III) 2,2′,2″-(10-(2-(4-(N-(2-(2-(2-carboxyethoxy)ethoxy)ethyl)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido) piperidin-1-yl)-2-oxoethyl)-1,4,7,10-tetraazacyclododecane-1,4,7-triyl)triacetate (14.9 mg, 10.5 μmol, 74.2%) as a white solid. Calc'd for C₆₂H₇₇C₁₂InN10017:1418.4, found [M+H]⁺: 1419.6.

### Example 64: Indium Complex of Compound 17 (Compound 17A)

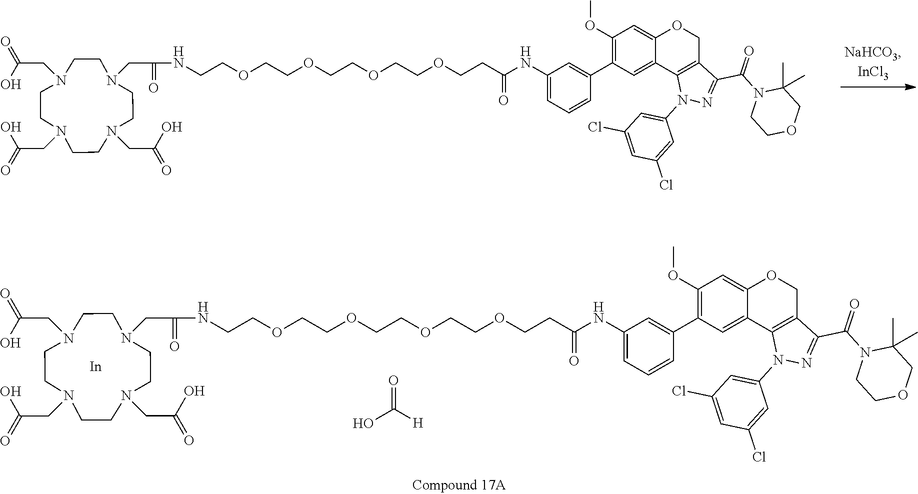

Into an 8-mL vial, was placed a mixture of 2,2′,2″-(10-(18-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,18-dioxo-6,9,12, 15-tetraoxa-3-azaoctadecyl)-1,4,7,10-tetraazacyclododecane-1,4,7-triyl)triacetic acid (from Example 17, 8 mg, 1 Eq, 7 μmol), indium (III) chloride (4 mg, 3 Eq, 0.02 mmol), sodium bicarbonate (3 mg, 1 μL, 5 Eq, 0.04 mmol), H₂O(0.5 mL) and ACN (1.0 mL). The reaction mixture was stirred at 80° C. for 2 hours. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide Compound 17A (4.6 mg, 3.4 μmol, 50%) as an off-white solid. Calc'd for C₅₈H₇₄C₁₂InN9018:1369.4, found [M+H−FA]⁺: 1324.6.

### Example 65: Indium Complex of Compound 23 (Compound 23A)

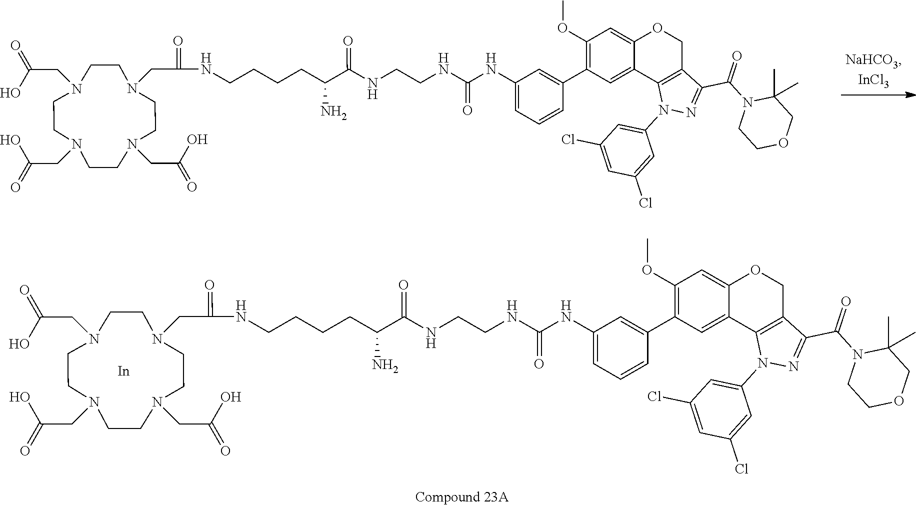

Into a 8-mL vial, was placed a mixture of (R)-2,2′,2″-(10-(2-((5-amino-6-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-6-oxohexyl)amino)-2-oxoethyl)-1,4,7,10-tetraazacyclododecane-1,4,7-triyl)triacetic acid (from Example 25, 25 mg, 1 Eq, 21 μmol), indium (III) chloride (15 mg, 3.8 Eq, 81 μmol), sodium bicarbonate (12 mg, 5.6 uL, 6.7 Eq, 0.14 mmol), H₂O(0.5 mL) and ACN (0.5 mL). The reaction mixture was stirred at 80° C. for an additional 2 hours. The crude product was purified by Prep-HPLC using the following conditions: Column: SunFire prep OBD 19*150 mm 5 μm; Mobile Phase A: Water (0.05% FA); Mobile Phase B: ACN; Gradient: 25% B to 65% B in 8 min; Flow rate: 20 mL/min; Wave Length: 220 nm. The collected fractions were dried by lyophilization to provide indium (III) (R)-2,2′,2″-(10-(2-((5-amino-6-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-6-oxohexyl)amino)-2-oxoethyl)-1,4,7,10-tetraazacyclododecane-1,4,7-triyl)triacetate (14.4 mg, 11.1 μmol, 53%) as a yellow oil. Calc'd for C₅₅H₆₉C₁₂InN12013:1290. 4, found [M+H]⁺: 1291.6, 1293.7.

### Example 66: Indium Complex of Compound 24 (Compound 24A)

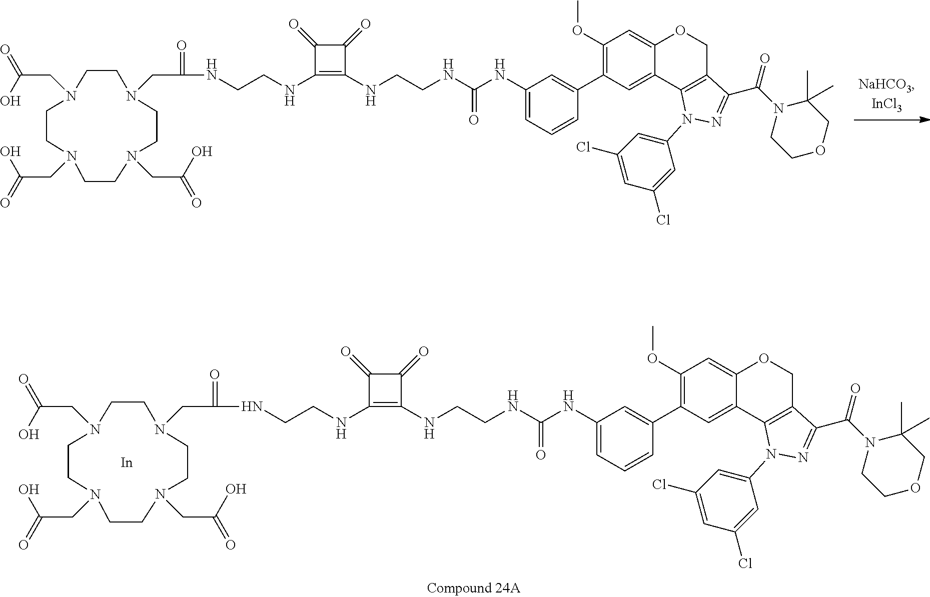

Into an 8-mL vial, was placed a mixture of 2,2′,2″-(10-(2-((2-((2-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,4-dioxocyclobut-1-en-1-yl)amino)ethyl)amino)-2-oxoethyl)-1,4,7,10-tetraazacyclododecane-1,4,7-triyl)triacetic acid (from Example 26, 14.2 mg, 1 Eq, 11.9 μmol), sodium bicarbonate (5.1 mg, 2.4 μL, 5.1 Eq, 61 μmol), indium (III) chloride (8.86 mg, 4.00 Eq, 47.7 μmol), MeCN (0.6 mL) and H₂O(0.3 mL). The reaction mixture was stirred at 80° C. for 2 hours. The mixture was directly purified by MPLC with the following conditions: Column, WelFlash™, C18 120 g, Spherical 20-40 μm; Mobile phase, Water (0.05% NH₃.H₂O) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were dried by lyophilization to provide indium (III) 2,2′,2″-(10-(2-((2-((2-((2-(3-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)ureido)ethyl)amino)-3,4-dioxocyclobut-1-en-1-yl)amino)ethyl)amino)-2-oxoethyl)-1,4,7,10-tetraazacyclododecane-1,4,7-triyl)triacetate (5.2 mg, 4.0 μmol, 33%) as a white solid. Calc'd for C₅₅H₆₃C₁₂InN12014:1300.3, found [M+H]⁺: 1301.4.

### Example 67: Indium Complex of Compound 21 (Compound 21A)

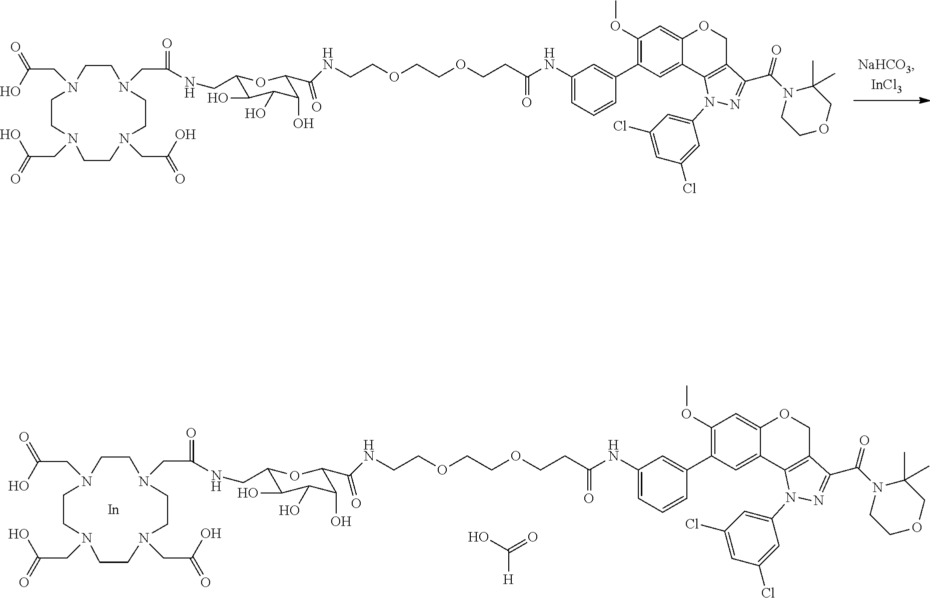

Into an 8 mL flask was added a mixture of 2,2′,2″-(10-(2-((((2S,3R,4R,5R,6S)-6-((2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid (25 mg, 1 Eq, 19 μmol), sodium bicarbonate (8 mg, 4 μL, 5 Eq, 0.1 mmol), indium trichloride (15 mg, 4.3 μL, 3.6 Eq, 68 μmol), ACN (0.2 mL), and water (0.1 mL). The mixture was stirred for 1 hour at 80° C. The mixture was diluted with 4 mL of DMSO, filtered and the filtrate was purified by Prep-HPLC with the following conditions (Prep-HPLC-007): Column, SunFire Prep C18 OBD Column, 19*150 mm 5 μm 10 nm; mobile phase, Water (0.05% FA) and ACN (30% ACN up to 85% in 16 min); Total flow rate, 20 mL/min; Detector, UV 220 nm. Purification provided indium (III) 2,2′,2″-(10-(2-((((2S,3R,4R,5R,6S)-6-((2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetate 1FA salt (9 mg, 6 μmol, 30%) as a white solid. Calc'd for C₆₁H₇₇C₁₂InN10021:1470.36, found [M+H−FA]⁺: 1425.4, 1427.4.

### Example 68: Indium Complex of Compound 22 (Compound 22A)

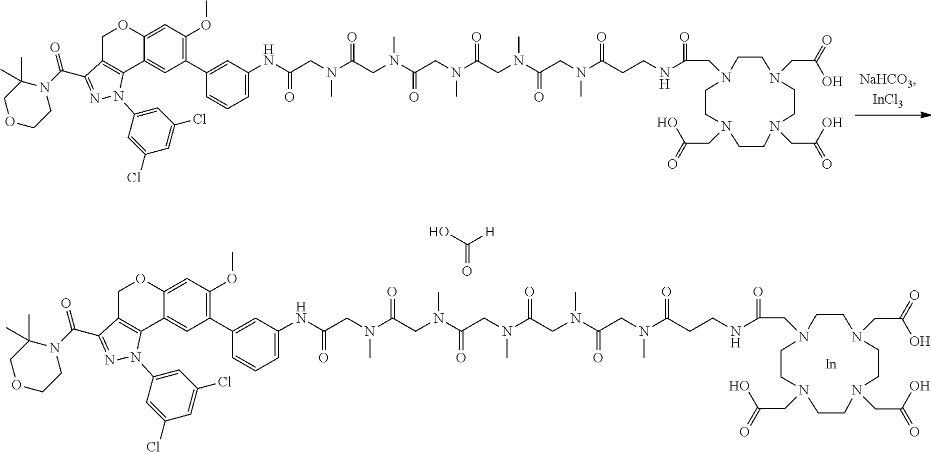

Into an 8 mL flask was added a mixture of 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,6,9,12, 15-pentamethyl-1,4,7,10, 13, 16,20-heptaoxo-3,6,9,12, 15,19-hexaazahenicosan-21-yl)-1,4,7,10-tetraazacyclododecane-1,4,7-triyl)triacetic acid (25 mg, 1 Eq, 18 μmol), sodium bicarbonate (8 mg, 4 μL, 5 Eq, 0.1 mmol), indium trichloride (15 mg, 4.3 uL, 3.8 Eq, 68 μmol), ACN (0.2 mL), and water (0.1 mL). The mixture was stirred for 1 hour at 80° C. The mixture was diluted with DMSO(4 mL), filtered and the filtrate was purified by Prep-HPLC with the following conditions (Prep-HPLC-007): Column, SunFire Prep C18 OBD Column, 19*150 mm 5 μm 10 nm; mobile phase, Water (0.05% FA) and ACN (30% ACN up to 75% in 18 min); Total flow rate, 20 mL/min; Detector, UV 220 nm. Purification provided indium (III) 2,2′,2″-(10-(1-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3,6,9,12,15-pentamethyl-1,4,7,10, 13, 16,20-heptaoxo-3,6,9, 12, 15, 19-hexaazahenicosan-21-yl)-1,4, 7,10-tetraazacyclododecane-1,4,7-triyl)triacetate 1FA salt (17.3 mg, 11.2 μmol, 62%) as a white solid. Calc'd for C₆₅H₈₃C₁₂InN14019:1548.44, found [M+H−FA]⁺: 1503.3, 1505.3.

### Example 69: Indium Complex of Compound 26 (Compound 26A)

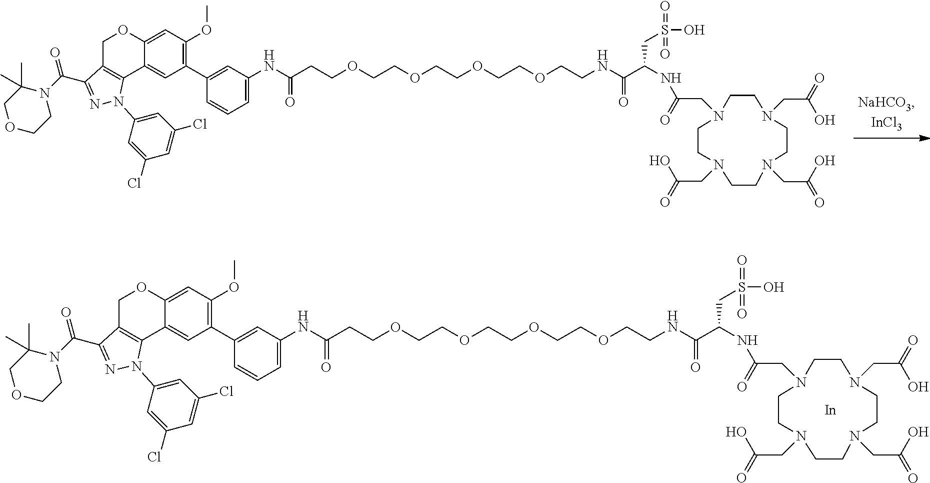

Into an 8 mL flask was added a mixture of (R)-2,2′,2″-(10-(21-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,5,21-trioxo-4-(sulfomethyl)-9,12, 15,18-tetraoxa-3,6-diazahenicosyl)-1,4,7,10-tetraazacyclododecane-1,4,7-triyl)triacetic acid (15 mg, 1 Eq, 11 μmol), sodium bicarbonate (5 mg, 2 L, 5 Eq, 0.06 mmol), indium trichloride (8 mg, 2 μL, 3 Eq, 0.04 mmol), ACN (0.2 mL), and water (0.1 mL). The mixture was stirred for 1 hour at 80° C. The mixture was diluted with DMSO(4 mL), filtered and the filtrate was purified by Prep-HPLC using the following conditions (Prep-HPLC-007): Column, SunFire Prep C18 OBD Column, 19*150 mm 5 μm 10 nm; mobile phase, Water (0.05% NH₃H₂O) and ACN (30% ACN up to 75% in 15 min); Total flow rate, 20 mL/min; Detector, UV 220 nm. Purification provided indium (III) (R)-2,2′,2″-(10-(21-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,5,21-trioxo-4-(sulfomethyl)-9,12,15, 18-tetraoxa-3,6-diazahenicosyl)-1,4,7,10-tetraazacyclododecane-1,4, 7-triyl)triacetate (5.9 mg, 4.0 μmol, 36%) as a white solid. Calc'd for C₆₀H₇₇C₁₂InN10020S: 1474.35, found [M+H]⁺: 1475.6, 1477.6.

### Example 70: (8-(5-aminopyridin-3-yl)-6-chloro-1-(3,5-dichlorophenyl)-7-methoxy-1,4dihydrochromeno|4,3-c]pyrazol-3-yl) (3,3-dimethylmorpholino) methanone

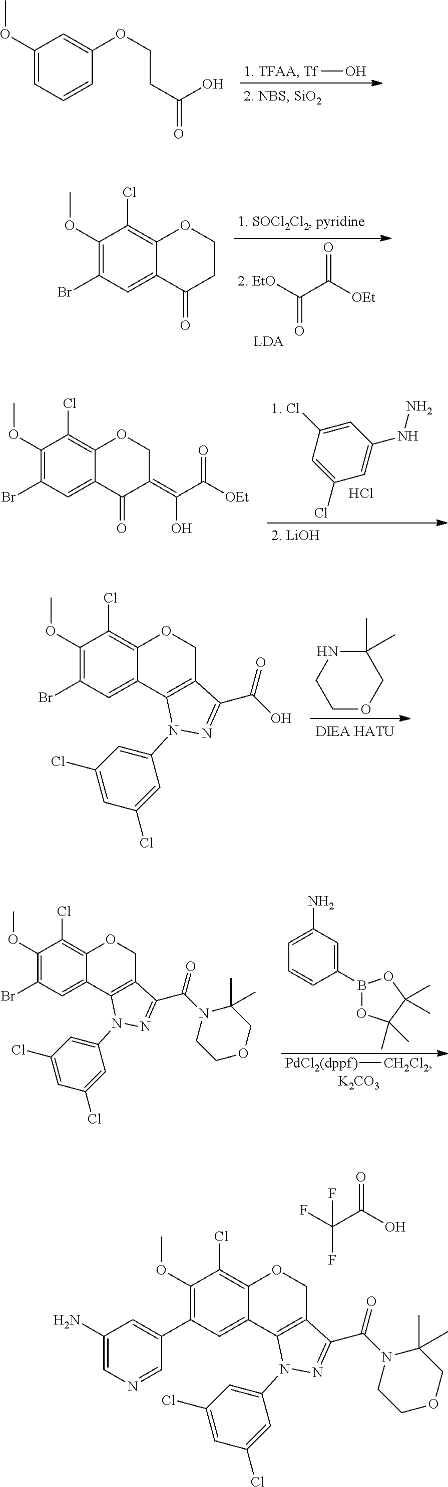

Step 1: Into a 1-L three mouth bottle, was placed a mixture of 3-(3-methoxyphenoxy) propanoic acid (50 g, 1 Eq, 0.25 mol), TFAA (74 g, 50 mL, 1.4 Eq, 0.35 mol), Tf-OH (85 g, 50 mL, 2.2 Eq, 0.57 mol) and DCM (500 mL). The reaction mixture was stirred at 25° C. for 2 hours. The mixture was diluted with water (500 mL), extracted with DCM (200 mL×3), then the combined organic layers were washed with NaHCO₃ (100 mL×2) and brine (200 mL), dried over anhydrous Na₂SO₄ and concentrated under reduced pressure. The crude product was purified by MPLC with the following conditions: Silica gel column 300 g, PE/EtOAc system, the ratio of EtOAc from 0% to 85% in 70 min, Flow rate: 120 mL/min; Wave Length: 254 nm. The collected fractions were concentrated under reduced pressure to provide 7-methoxychroman-4-one (30 g, 0.17 mol, 66%) as an off-white solid. Calc'd for C₁₀H₁₀O₃:178.1, found [M+H]⁺: 179.1.

Step 2: Into a 1 L three mouth round bottom flask, was placed a mixture of 7-methoxychroman-4-one (30 g, 1 Eq, 0.17 mol), NBS (36 g, 1.2 Eq, 0.20 mol), hydrogen [hydroxy (oxo) silyl]silicate (4 g, 2 mL, 0.4 Eq, 0.07 mol), MeCN (100 mL), and Et₂O(300 mL). The reaction mixture was stirred at 25° C. for 4 hours. The mixture was diluted with 500 mL of water, extracted with EtOAc (50 mL×3), then the combined organic layers were washed with water (100 mL×2), brine (100 mL), dried over anhydrous Na₂SO₄ and concentrated under reduced pressure. The crude product was purified by MPLC with the following conditions: Silica gel column 330 g, PE/EtOAc system, the ratio of EtOAc from 0% to 85% in 75 min, Flow rate: 120 mL/min; Wave Length: 254 nm. The collected fractions were concentrated under reduced pressure to provide 6-bromo-7-methoxychroman-4-one as an off-white solid. Calc'd for C₁₄H₁₂BrCIO6:255.97, found [M+H]⁺: 257.0.

Step 3: Into a 40-mL vial, was placed a mixture of 6-bromo-7-methoxychroman-4-one (900 mg, 1 Eq, 3.50 mmol) and DCM (10 mL). The reaction mixture was stirred at 0° C. with ice/water bath, sulfuryl dichloride (614 mg, 1.30 Eq, 4.55 mmol)_{w} as added dropwise, then pyridine (415 mg, 1.50 Eq, 5.25 mmol)_{w} as added. The reaction mixture was stirred from 0° C. to 25° C. for 2 hours. Combined with the first batch, the mixture was quenched with 30 mL of water, extracted with DCM (30 mL×3), the combined organic layers were washed with brine (30 mL), dried over anhydrous Na₂SO₄ and concentrated under reduced pressure. The crude product was purified by MPLC using the following conditions: Silica gel column 40 g, PE/EtOAc system, the ratio of EtOAc from 0% to 20% in 15 min, Flow rate: 40 mL/min; Wave Length: 254 nm. The collected fractions were concentrated to provide 6-bromo-8-chloro-7-methoxychroman-4-one (890 mg, 2.9 mmol, 83%, 95% Purity) as a white solid. Calc'd for C₁₀H&BrCIO3:289.9, found [M+H]: 290.9, 292.9.

Step 4: Into a 50 mL three-neck round bottom flask, purged and maintained with an inert atmosphere of nitrogen, was placed a mixture of 6-bromo-8-chloro-7-methoxychroman-4-one (800 mg, 1 Eq, 2.74 mmol) and THF (16 mL), to which diethyl oxalate was added dropwise (602 mg, 1.50 Eq, 4.12 mmol) (1.0M in THF) at −78° C., The reaction mixture was stirred at −78° C. for 30 min and diethyl oxalate (15 mg, 1.5 Eq, 0.10 mmol)_{w} as added dropwise. The reaction mixture was stirred at −78° C. for 5 hours. The mixture was quenched with 100 mL of NH₄Cl aqueous solution, extracted with EtOAc (50 mL×3), the combined organic layers were washed with brine (100 mL), dried over anhydrous Na₂SO₄ and concentrated under reduced pressure. The crude product was purified by MPLC with the following conditions: Silica gel column 40 g, PE/EtOAc system, the ratio of EtOAc from 0% to 85% in 30 min, Flow rate: 40 mL/min; Wave Length: 254 nm. The collected fractions were concentrated under reduced pressure to provide ethyl (Z)-2-(6-bromo-8-chloro-7-methoxy-4-oxochroman-3-ylidene)-2-hydroxyacetate (200 mg, 0.41 mmol, 15%, 80% Purity) as a yellow solid. Calc'd for C₁₄H₁₂BrCIO6:390.0, found [M+H]: 391.1, 393.1.

Step 5: Into a 20 ml vial added ethyl (Z)-2-(6-bromo-8-chloro-7-methoxy-4-oxochroman-3-ylidene)-2-hydroxyacetate (200 mg, 1 Eq, 511 μmol) in t-BuOH (0.4 mL) and AcOH (6 mL), to which was added (3,5-dichlorophenyl) hydrazine hydrochloride (100 mg, 0.917 Eq, 468 μmol) at RT under N₂. The mixture was stirred at 100° C. for 16 hours then the mixture was concentrated in vacuo. The residue was dissolved in EtOAc (40 mL), then washed with water (20 mL), brine (20 mL), dried over anhydrous sodium sulfate, and concentrated in vacuo. The residue was washed with 20 mL PE to afford ethyl 8-bromo-6-chloro-1-(3,5-dichlorophenyl)-7-methoxy-1,4-dihydrochromeno[4,3-c]pyrazole-3-carboxylate (200 mg, 0.36 mmol, 70%, 95% Purity) as an off-white solid. Calc'd for C₂₀H₁₄BrC13N₂O₄:529.92, found [M+H]: 531.0, 533.0

Step 6: Into a 20-mL vial, was placed a mixture of ethyl 8-bromo-6-chloro-1-(3,5-dichlorophenyl)-7-methoxy-1,4-dihydrochromeno[4,3-c]pyrazole-3-carboxylate (100 mg, 1 Eq, 188 μmol), LiOH (45 mg, 10 Eq, 1.9 mmol), THF (5 mL) and water (0.5 mL). The reaction mixture was stirred at 25° C. for 16 hours. The reaction mixture was concentrated under reduced pressure to remove most of the THF, the residue was diluted with water (5 mL), the pH value was adjusted to 6.0 by addition of a saturated NaHSO+solution, then the resulting precipitate was collected by filtration and dried to afford 8-bromo-6-chloro-1-(3,5-dichlorophenyl)-7-methoxy-1,4-dihydrochromeno[4,3-c]pyrazole-3-carboxylic acid (70 mg, 0.12 mmol, 67%, 90% Purity) as an off-white solid. Calc'd for C18H₁₀BrC13Ns04:501.89, found [M+H]: 502.9, 504.9.

Step 7: Into an 8-mL vial, was placed a mixture of 8-bromo-6-chloro-1-(3,5-dichlorophenyl)-7-methoxy-1,4-dihydrochromeno[4,3-c]pyrazole-3-carboxylic acid (65 mg, 1 Eq, 0.13 mmol), DIEA (54 mg, 73 μL, 3.2 Eq, 0.42 mmol), HATU (63 mg, 1.3 Eq, 0.17 mmol) and DMF (1 mL). The reaction mixture was stirred at 25° C. for 10 minutes, then 3,3-dimethylmorpholine (24 mg, 1.6 Eq, 0.21 mmol)_{w} as added and the reaction mixture was stirred at 25° C. for 2 hours. The mixture was directly purified by MPLC using the following conditions: Column, C18 120 g, Spherical 20-40 μm; Mobile phase, Water (0.1% TFA) and ACN (5% ACN to 5% ACN in 1 min, 30% ACN up to 98% in 6 min, 98% ACN to 98% in 3 min); Total flow rate, 70 mL/min; Detector, UV 220 nm. This resulted in (8-bromo-6-chloro-1-(3,5-dichlorophenyl)-7-methoxy-1,4-dihydrochromeno[4,3-c]pyrazol-3-yl) (3,3-dimethylmorpholino) methanone (47 mg, 78 μmol, 61%) as an off-white solid. Calc'd for C₂₄H₂₁BrC13N₃O₄:598.98, found [M+H]: 600.1, 602.1.

Step 8: Into an 8 mL vial, maintained with an inert atmosphere of nitrogen, was placed a mixture of (8-bromo-6-chloro-1-(3,5-dichlorophenyl)-7-methoxy-1,4-dihydrochromeno[4,3-c]pyrazol-3-yl) (3,3-dimethylmorpholino) methanone (45 mg, 1 Eq, 75 μmol), 3-(4,4,5,5-tetramethyl-1,3,2-dioxaborolan-2-yl)aniline (16 mg, 0.98 Eq, 73 μmol), PdC12 (dppf)-CH₂C₁₂ (5 mg, 0.08 Eq, 6 μmol), K₂CO₃ (31 mg, 3.0 Eq, 0.22 mmol), 1,4-Dioxane (0.8 mL) and water (0.08 mL). The reaction mixture was stirred at 100° C. for 0.5 hours. The crude product was purified by Prep-HPLC with the following conditions: Column: SunFire prep OBD 19*150 mm 5 μm; Mobile Phase A: Water (0.05% TFA); Mobile Phase B: ACN; Gradient: 20% B to 60% B in 8 min; Flow rate: 20 mL/min; Wave Length: 220 nm. The collected fractions were dried by lyophilization to provide (8-(3-aminophenyl)-6-chloro-1-(3,5-dichlorophenyl)-7-methoxy-1,4-dihydrochromeno[4,3-c]pyrazol-3-yl) (3,3-dimethylmorpholino) methanone 2,2,2-trifluoroacetate (22.4 mg, 30 μmol, 40%, 97% Purity) as an off-white solid. Calc'd for C₃₂H₂₈C₁₃F3N₄O₆:726. 10, found [M+H−TFA]⁺: 614.1, 616.1.

### Example 71:2,2′,2″-(10-((4R,7R,10R,13R,16R,19R,22R)-23-amino-13,16-bis(2-amino-2oxoethyl)-7-(4-(4-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-4-oxobutanamido)butyl)-10,19-bis(hydroxymethyl)-4,22-dimethyl-2,5,8,11,14,17,20,23-octaoxo-3,6,9,12, 15,18,21-heptaazatricosyl)-1,4,7,10-tetraazacyclododecane-1,4,7-triyl)triacetic acid (Compound 101)

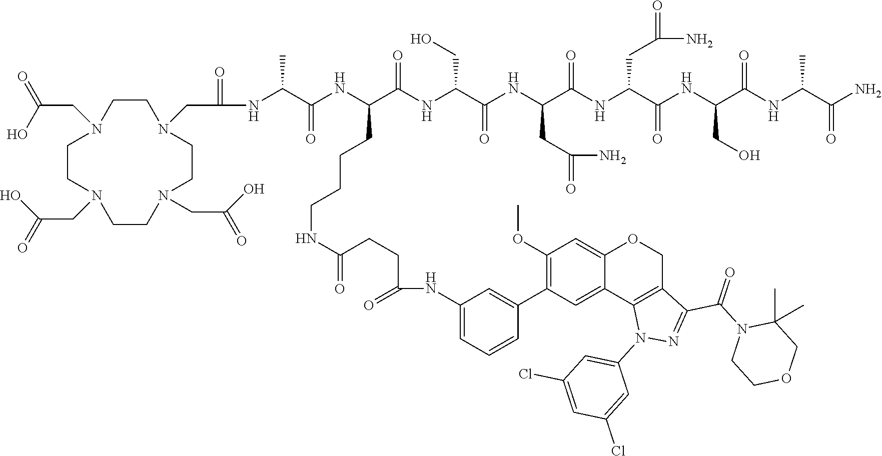 [SMILES: COc1cc2c(cc1-c1cccc(NC(=O)CCC(=O)NCCCC[C@@H](NC(=O)[C@@H](C)NC(=O)CN3CCN(CC(=O)O)CCN(CC(=O)O)CCN(CC(=O)O)CC3)C(=O)N[C@H](CO)C(=O)N[C@H](CC(N)=O)C(=O)N[C@H](CC(N)=O)C(=O)N[C@H](CO)C(=O)N[C@H](C)C(N)=O)c1)-c1c(c(C(=O)N3CCOCC3(C)C)nn1-c1cc(Cl)cc(Cl)c1)CO2]

Examples 71-75 may be synthesized according to the following general solid phase peptide synthesis(SPPS) Procedure:

The automated SPPS was executed with a Syro II peptide synthesizer (Biotage).

Step 1: Attachment of the first amino acid on CTC resin: Into a sealed tube were added 2-chlorotrityl chloride resin (1.1 mmol/g) and DCM (10 mL/g resin) at room temperature under nitrogen atmosphere. The resulting mixture was swollen for 15 min at room temperature under nitrogen atmosphere. The resin was washed with CH₂Cl₂ (3×100 mL). To the resin was added appropriate amino acid (1.00 Eq), DIEA (1.00 Eq) and DCM (10 mL/resin). The mixture was agitated for 5 min at room temperature under nitrogen atmosphere. Then DIEA (1.5 Eq)_{w} as added. After the mixture was agitated for another 60 min, MeOH (0.8 mL/g resin)_{w} as added to endcap any remaining reactive trityl groups. The resin was filtered and washed twice with CH₂C₁₂ (10 mL/g resin), twice with DMF and three times with MeOH. The resin was dried in vacuo. The loading efficiency was calculated by weight gain.

Step 2: Resin swelling: the resin (100 mg/tube)_{w} as swelled by NMP (1 mL/tube) for 15 min at room temperature under nitrogen atmosphere. Then, the resin was washed 4 times with NMP (1 mL/tube). Parameter: reaction time: 15 min, vortex time: 15 s, break time: 1 min, temperature: room temperature, empty time: 1 min.

Step 3: Fmoc de-protection: the resin (100 mg/tube)_{w} as treated with 20% of piperidine (1 mL) for 20 min at room temperature under nitrogen atmosphere. The resin was then washed 4 times with NMP (1 mL/tube). Parameter: reaction time: 20 min, vortex time: 15 s, break time: 1 min, temperature: room temperature, empty time: 1 min.

Step 4: HATU coupling: the resin (100 mg/tube)_{w} as treated with the mixture of amino acid (4 Eq), HATU (4 Eq) and DIPEA (8 Eq) in NMP (1 mL) for 45 min at 30° C. under nitrogen atmosphere. Then, the resin was washed 4 times with NMP (1 mL/tube). Parameter: reaction time: 45 min, vortex time: 15 s, break time: 3 min, temperature: 30° C., empty time: 1 min. Steps 3 and 4 are repeated to obtain the appropriate length peptide.

Step 5: Capping: the resin (100 mg/tube)_{w} as capped with the solution (v v v Ac20/DIPEA/NMP=31.5:8.5:160) for 1 h at room temperature under nitrogen atmosphere. Then, the resin was washed 4 times with NMP (1 mL/tube). Parameter: reaction time: 1 h, vortex time: 30 s, break time: 1 min, temperature: room temperature, empty time: 1 min.

Step 6: A) Cleavage & purification for peptides synthesized with Rink amide resin and Wang resin: the crude peptide was cleaved from the resin with the solution (vvvy TFA/H₂O/TIS/DODT=37:1:1:1) for 2 h at room temperature. The mixture was filtered, and the filtrate was concentrated under reduced pressure. The residue was precipitated with cold ether and purified with prep-HPLC. B) Cleavage & purification for peptides synthesized with CTC resin: the crude peptide was cleaved from the resin with 1, 1, 1,3,3,3-hexafluoropropan-2-ol: DCM (1:4, v/v) for 30 min at room temperature. The crude product was purified by Prep-HPLC.

### Example 72:2,2′,2″-(10-((4R,7R,10R,13R,16R,19R,22R)-23-amino-10,16-bis(2-amino-2oxoethyl)-13-(4-(4-((3-(1-(3,5-dichlorophenyl)-3-(3,3-dimethylmorpholine-4-carbonyl)-7-methoxy-1,4-dihydrochromeno 4,3-c|pyrazol-8-yl)phenyl)amino)-4-oxobutanamido)butyl)-7,19-bis(hydroxymethyl)-4,22-dimethyl-2,5,8,11,14,17,20,23-octaoxo-3,6,9,12, 15,18,21-heptaazatricosyl)-1,4,7,10-tetraazacyclododecane-1,4,7-triyl)triacetic acid (Compound 102)

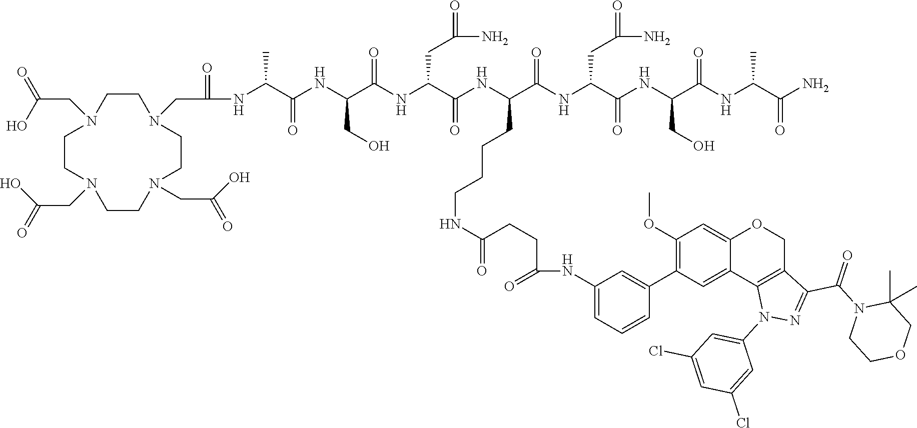 [SMILES: COc1cc2c(cc1-c1cccc(NC(=O)CCC(=O)NCCCC[C@@H](NC(=O)[C@@H](CC(N)=O)NC(=O)[C@@H](CO)NC(=O)[C@@H](C)NC(=O)CN3CCN(CC(=O)O)CCN(CC(=O)O)CCN(CC(=O)O)CC3)C(=O)N[C@H](CC(N)=O)C(=O)N[C@H](CO)C(=O)N[C@H](C)C(N)=O)c1)-c1c(c(C(=O)N3CCOCC3(C)C)nn1-c1cc(Cl)cc(Cl)c1)CO2]

### Example 73:2,2′,2″-(10-((4R,7R,10R,13R,16R,19R)-19-(((R)-1-amino-1-oxopropan-2yl) carbamoyl)-10,13-bis(2-amino-2-oxoethyl)-28-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-7,16-bis(hydroxymethyl)-4-methyl-2,5,8,11,14,17,25,28-octaoxo-3,6,9,12,15,18,24-heptaazaoctacosyl)-1,4,7,10-tetraazacyclododecane-1,4,7-triyl)triacetic acid (Compound 103)

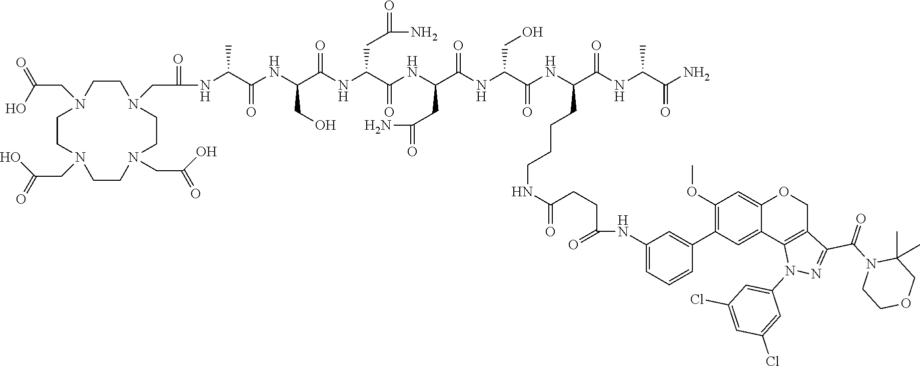 [SMILES: COc1cc2c(cc1-c1cccc(NC(=O)CCC(=O)NCCCC[C@@H](NC(=O)[C@@H](CO)NC(=O)[C@@H](CC(N)=O)NC(=O)[C@@H](CC(N)=O)NC(=O)[C@@H](CO)NC(=O)[C@@H](C)NC(=O)CN3CCN(CC(=O)O)CCN(CC(=O)O)CCN(CC(=O)O)CC3)C(=O)N[C@H](C)C(N)=O)c1)-c1c(c(C(=O)N3CCOCC3(C)C)nn1-c1cc(Cl)cc(Cl)c1)CO2]

### Example 74: (6R,9R,12R,15R,18R,21R)-1-amino-9-(3((amino (iminio)methyl)amino) propyl)-21-carbamoyl-23-carboxy-15,18-bis(2-carboxyethyl)-12-(4-(4-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-4-oxobutanamido)butyl)-7,10,13,16,19-pentaoxo-6-((R)-2-(2-(4,7,10-tris(carboxymethyl)-1,4,7,10-tetraazacyclododecan-1-yl)acetamido) propanamido)-2,8,11,14,17,20-hexaazatricosan-1-iminium (Compound 104)

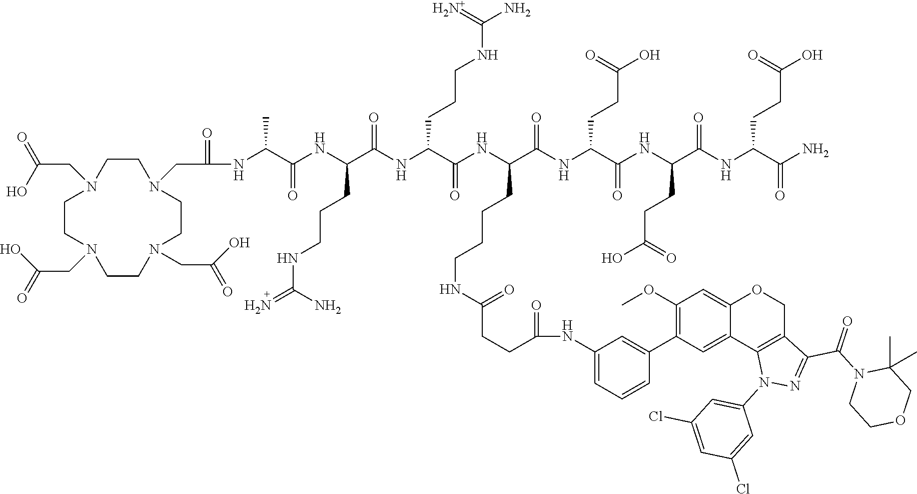 [SMILES: COc1cc2c(cc1-c1cccc(NC(=O)CCC(=O)NCCCC[C@@H](NC(=O)[C@@H](CCCNC(N)=[NH2+])NC(=O)[C@@H](CCCNC(=N)N)NC(=O)[C@@H](C)NC(=O)CN3CCN(CC(=O)O)CCN(CC(=O)O)CCN(CC(=O)O)CC3)C(=O)N[C@H](CCC(=O)O)C(=O)N[C@H](CCC(=O)O)C(=O)N[C@H](CCC(=O)O)C(N)=O)c1)-c1c(c(C(=O)N3CCOCC3(C)C)nn1-c1cc(Cl)cc(Cl)c1)CO2]

### Example 75: (4R,7R,10R,13R,16R)-4-(((R)-1-amino-1-oxopropan-2-yl) carbamoyl)-10-(4-(4((3-(1-(3,5-dichlorophenyl)-3-(3,3-dimethylmorpholine-4-carbonyl)-7-methoxy-1,4-dihydrochromeno|4,3-clpyrazol-8-yl)phenyl)amino)-4-oxobutanamido)butyl)-7,13-dimethyl-6,9,12,15-tetraoxo-16-((R)-2-(2-(4,7,10-tris(carboxymethyl)-1,4,7,10-tetraazacyclododecan-1-yl)acetamido) propanamido)-5,8,11,14-tetraazanonadecanedioic acid (Compound 86)

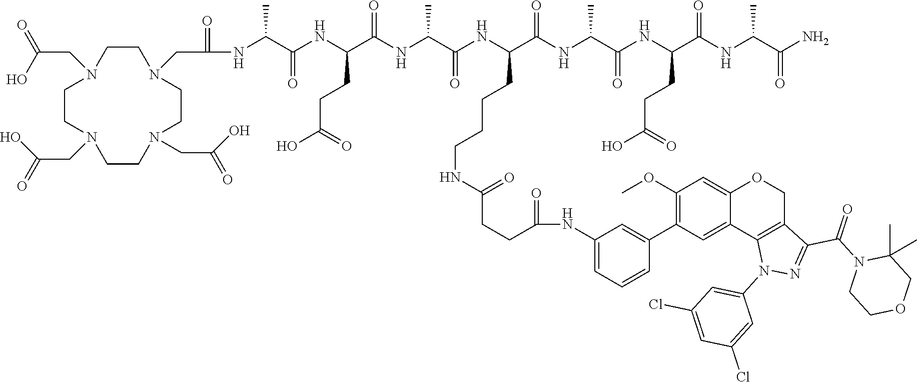 [SMILES: COc1cc2c(cc1-c1cccc(NC(=O)CCC(=O)NCCCC[C@@H](NC(=O)[C@@H](C)NC(=O)[C@@H](CCC(=O)O)NC(=O)[C@@H](C)NC(=O)CN3CCN(CC(=O)O)CCN(CC(=O)O)CCN(CC(=O)O)CC3)C(=O)N[C@H](C)C(=O)N[C@H](CCC(=O)O)C(=O)N[C@H](C)C(N)=O)c1)-c1c(c(C(=O)N3CCOCC3(C)C)nn1-c1cc(Cl)cc(Cl)c1)CO2]

### Synthesis of Intermediate C:

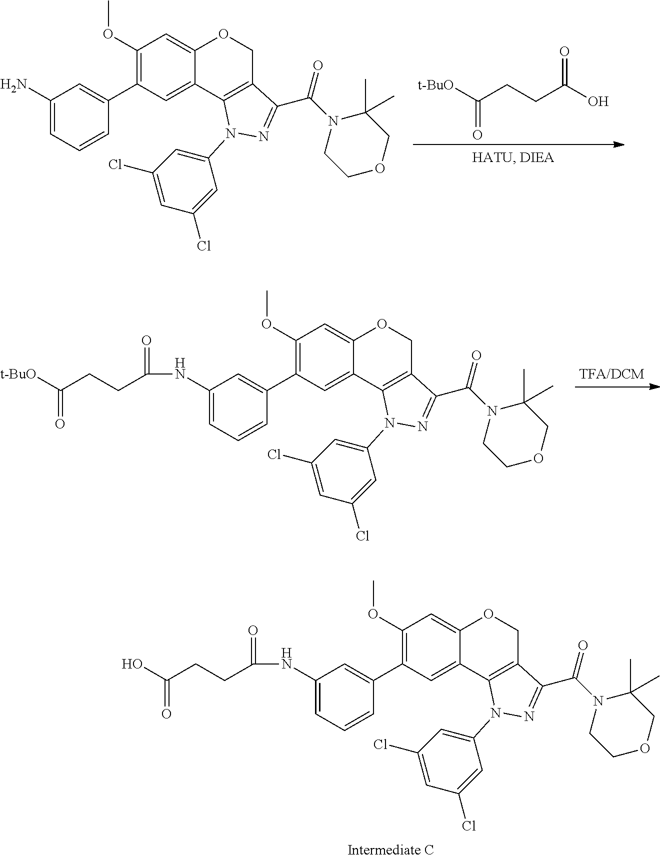

Step 1: Into a 40-mL vial, was placed with a mixture of 4-(tert-butoxy)-4-oxobutanoic acid (0.80 g, 1.0 Eq, 4.6 mmol), N-ethyl-N-isopropylpropan-2-amine (1.8 g, 3.0 Eq, 14 mmol), 2-(3H-[1,2,3]triazolo[4,5-b]pyridin-3-yl)-1,1,3,3-tetramethylisouronium hexafluorophosphate (V) (2.2 g, 1.3 Eq, 5.8 mmol) and DMF (30 mL). The reaction mixture was stirred at 20° C. for 10 minutes, then (8-(3-aminophenyl)-1-(3,5-dichlorophenyl)-7-methoxy-1,4-dihydrochromeno[4,3-c]pyrazol-3-yl) (3,3-dimethylmorpholino) methanone (500 mg, 1.00 Eq, 863 μmol)_{w} as added and the reaction mixture was stirred at 25° C. for an additional 1 hour. The mixture was directly purified by MPLC to provide tert-butyl 4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoate (3.2 g, 4.3 mmol, 95%) as a white solid. LCMS: (ESI, m/z): [M+H]⁺=735.2, 735.4.

Step 2: Into a 100-mL round bottom flask, was placed with a mixture of tert-butyl 4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoate (3.2 g, 1.0 Eq, 4.3 mmol) and DCM (40 mL), to which was added TFA (4 mL). The reaction mixture was stirred at 25° C. for 40 minutes. The mixture was concentrated under reduced pressure. The crude product was purified by Prep-HPLC with DAC. The crude product was purified by DAC-HPLC: Column, YMC-C18, 150-250 nm, 10 μm; Mobile phase, Water (0.05% TFA) and ACN (25% B to 65%

B in 8 min; Flow rate: 120 mL/min; UV detection wavelength: 220 nm. The collected fractions were pooled and lyophilized to afford 4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanoic acid (Intermediate C) (1.926 g, 2.834 mmol, 65%) as a white solid. LCMS: (ESI, m/z): [M+H]⁺=679.3, 681.3. ¹H NMR (300 MHz, DMSO—d6) δ 12.1 (br.s, 1H), 9.90 (s, 1H), 7.83 (s, 3H), 7.57 (s, 1H), 7.50 (d, J=8.1 Hz, 1H), 7.24 (t, J=7.9 Hz, 1H), 6.89-6.77 (m, 2H), 6.74 (s, 1H), 5.40 (s, 2H), 3.91 (d, J=5.5 Hz, 2H), 3.78-3.65 (m, 5H), 3.48-3.50 (m, 2H), 2.58-2.51 (m, 4H), 1.43 (s, 6H).

### Synthesis of Compound 86:

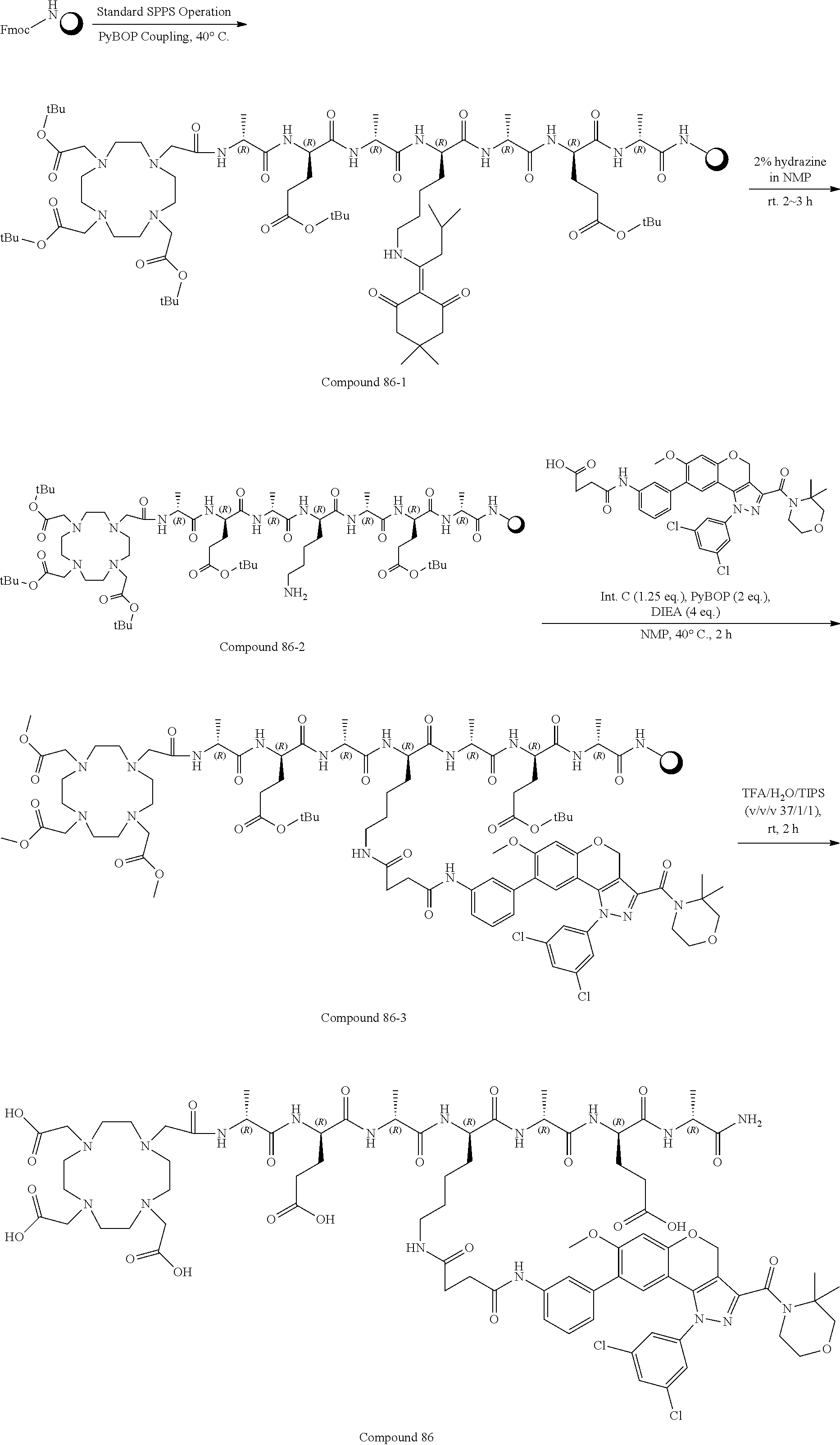

### Step 1: Synthesis of Compound 86-1

The automated SPPS was executed with a Syro II peptide synthesizer (Biotage).

1) Resin pre-swelling: The Rink amide resin (100 mg/tube, 100-200 mesh, 0.62 mmol/g, total 5 tubes)_{w} as swelled in NMP (1 mL/tube) for 15 min at room temperature under nitrogen atmosphere. The resin was then washed 4 times with NMP (1 mL/tube). Parameter: incubation time: 15 min, vortex time: 15 s, break time: 1 min, temperature: room temperature, empty time: 1 min.

SPPS coupling cycle: operation 2) to 3)

2) Fmoc de-protection: the resin (100 mg/tube, total 5 tubes)_{w} as treated with 20% of piperidine in NMP (1 mL) for 20 min at room temperature under nitrogen atmosphere. The resin was then washed 4 times with NMP (1 mL/tube). Parameter: reaction time: 20 min, vortex time: 15 s, break time: 1 min, temperature: room temperature, empty time: 1 min.

3) PyBOP coupling: the resin (100 mg/tube, total 5 tubes)_{w} as treated with the mixture of amino acid or DOTA-(OfBu) 3 (4 eq.), PyBOP (4 eq.) and DIPEA (8 eq.) in NMP (1 mL) for 45 min at 30° C. under nitrogen atmosphere. Then, the resin was washed 4 times with NMP (1 mL/tube). Parameter: reaction time: 45 min, vortex time: 15 s, break time: 3 min, temperature: 30° C., empty time: 1 min.

### Step 2: Synthesis of Compound 86-2

Compound 86-1 on resin (500 mg)_{w} as swelled with NMP (5 mL) for 15 min at room temperature. The solvent was then removed. To the resin (500 mg) in 25 mL reactor was added 2% hydrazine in NMP (5 mL) at room temperature under nitrogen atmosphere. The mixture was bubbled for 2 h and filtered, washed with NMP (5 mL×8). The reaction was monitored by LCMS.

### Step 3: Synthesis of Compound 86-3

To the resin (500 mg) in 25 mL reactor were added Intermediate C (1.25 eq.), PyBOP (2 eq.) and DIPEA (4 eq.) in NMP (5 mL). After the reaction mixture was incubated for 2 h at 40° C., the solvent was removed and the resin was washed with NMP (5 mL×6).

### Step 4: Synthesis of Compound 86

The resin was treated with the cleavage solution (v v v TFA/H₂O/TIS=37:1:1) for 2 h at room temperature. After filtration, the filtrate was concentrated under reduced pressure. The residue was precipitated with cold ether. After centrifugation, the solid was collected and purified by prep-HPLC. The desired fractions were combined and lyophilized to afford Compound 86 as a white solid (193.8 mg, 29.1%, 97.1% purity). Calc'd for C₇₈H₁₀₅C₁₂N₁₇O₂₄:1733.7, found [M+2H]²⁺: 868.6.

### Example 76: Indium Complex of Compound 32 (Compound 32A)

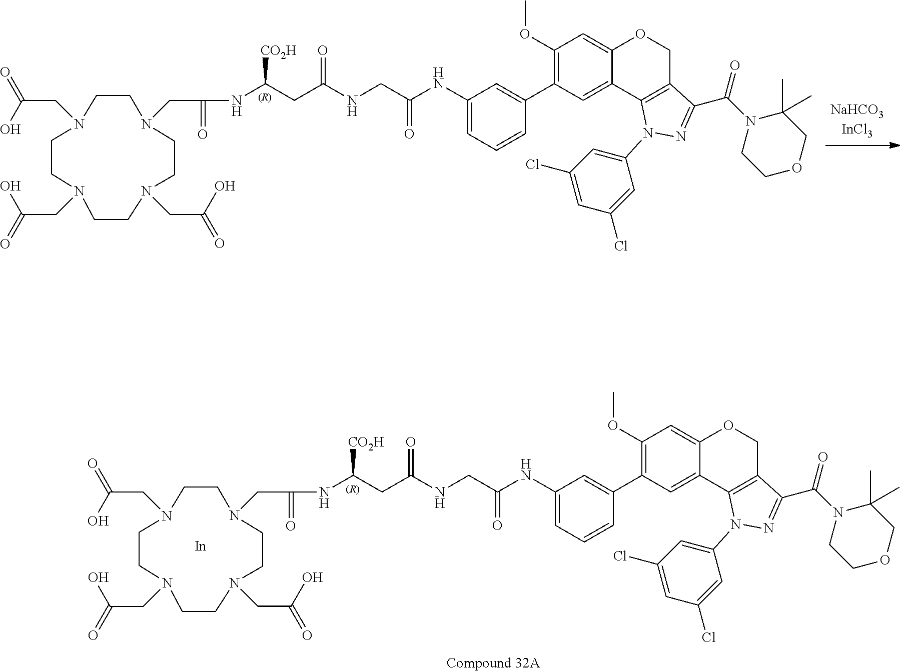

The title compound may be synthesized using a procedure similar to that for Example 49.

### Example 77: Indium Complex of Compound 44 (Compound 44A)

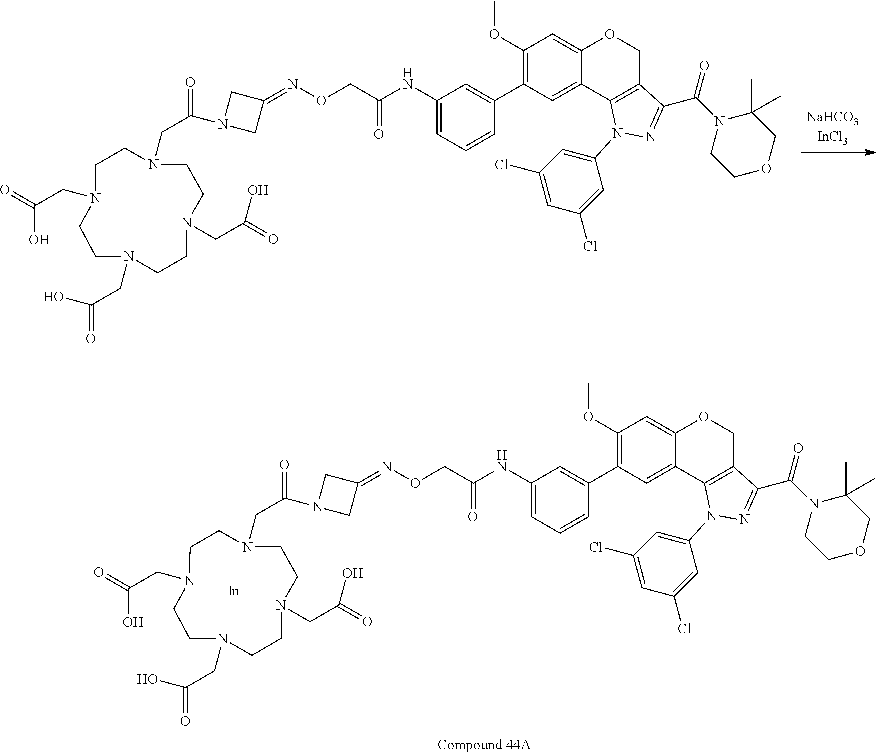

The title compound may be synthesized using a procedure similar to that for Example 49.

### Example 78: (S)-2,2′,2″-(10-(2-((3-(4-((5-(1-(3,5-difluorophenyl)-3-(3,3dimethylmorpholine-4-carbonyl)-7-methoxy-1,4-dihydrochromeno[4,3-c]pyrazol-8-yl) pyridin-3-yl)amino)-4-oxobutanamido)-1-oxo-1-(4-(2-sulfoethyl) piperazin-1-yl) propan-2-yl)amino)-2-oxoethyl)-1,4,7,10-tetraazacyclododecane-1,4,7-triyl)triacetic acid-formic acid (1/1) (Compound 49)

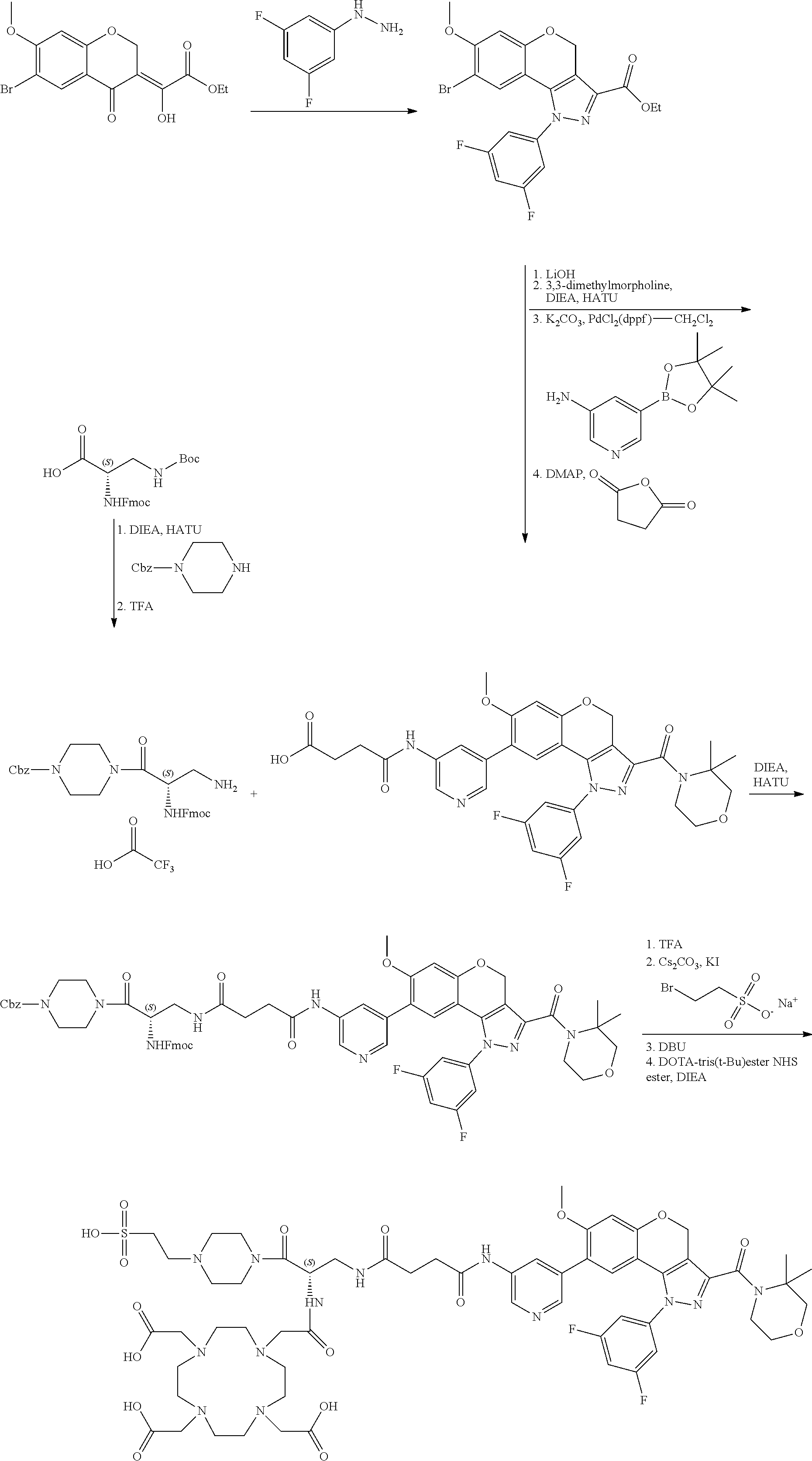

Step 1: ethyl (Z)-2-(6-bromo-7-methoxy-4-oxochroman-3-ylidene)-2-hydroxyacetate (8 g, 1 Eq, 0.02 mol)_{w} as treated with (3,5-difluorophenyl) hydrazine and AcOH in a manner similar to that Step 5 of Example 1 to provide ethyl 8-bromo-1-(3,5-difluorophenyl)-7-methoxy-1,4-dihydrochromeno[4,3-c]pyrazole-3-carboxylate as a light yellow solid. [M+H]=465.1.

Step 2: Ethyl 8-bromo-1-(3,5-difluorophenyl)-7-methoxy-1,4-dihydrochromeno[4,3-c]pyrazole-3-carboxylate (9.1 g, 1 Eq, 20 mmol), LiOH (0.47 g, 1 Eq, 20 mmol), EtOH (1000 mL) and H₂O(200 mL). The reaction mixture was stirred at 25° C. for 16 hours. The mixture was quenched with an aqueous solution of HCl until the pH value was adjusted to 6.0. The precipitated solids were collected by filtration and washed with ethyl acetate (3×300 ml) to provide 8-bromo-1-(3,5-difluorophenyl)-7-methoxy-1,4-dihydrochromeno[4,3-c]pyrazole-3-carboxylic acid (8.0 g, 16 mmol, 84%) as an off-white solid. [M+H]=437.0.

Step 3: Into a 250 ml three port round bottom flask, was placed with a mixture of 8-bromo-1-(3,5-difluorophenyl)-7-methoxy-1,4-dihydrochromeno[4,3-c]pyrazole-3-carboxylic acid (7 g, 1 Eq, 0.02 mol), DIEA (6 g, 8 mL, 3 Eq, 0.05 mol), HATU (7 g, 1 Eq, 0.02 mol) and DMF (70 mL). The reaction mixture was stirred at 20° C. for 10 minutes, then 3,3-dimethylmorpholine (2 g, 1 Eq, 0.02 mol)_{w} as added and the reaction mixture was stirred at 25° C. for an additional 1 hour. The mixture was diluted with water (250 mL), extracted with EtOAc (250 mL×3), then the combined organic layers were washed with water (250 mL×2) and brine (250 mL), dried over anhydrous Na₂SO₄ and concentrated under reduced pressure. The crude product was purified by MPLC to provide (8-bromo-1-(3,5-difluorophenyl)-7-methoxy-1,4-dihydrochromeno[4,3-c]pyrazol-3-yl) (3,3-dimethylmorpholino) methanone (6.0 g, 10 mmol, 60%) as an off-white solid. [M+H]=534.1, 536.1.

Step 4: (8-bromo-1-(3,5-difluorophenyl)-7-methoxy-1,4-dihydrochromeno[4,3-c]pyrazol-3-yl) (3,3-dimethylmorpholino) methanone was treated with 5-(4,4,5,5-tetramethyl-1,3,2-dioxaborolan-2-yl) pyridin-3-amine, 1,1′-Bis(di-t-butylphosphino) ferrocene palladium dichloride (137 mg, 0.0411 Eq, 210 μmol), and K₂CO₃ in a manner similar to Step 6 of Example 1 to provide (8-(5-aminopyridin-3-yl)-1-(3,5-difluorophenyl)-7-methoxy-1,4-dihydrochromeno[4,3-c]pyrazol-3-yl) (3,3-dimethylmorpholino) methanone (6.2 g, 9.7 mmol, 90%) as an off-white solid. [M+H]=548.2.

Step 5: Into a 40-mL vial, was placed a mixture of (8-(5-aminopyridin-3-yl)-1-(3,5-difluorophenyl)-7-methoxy-1,4-dihydrochromeno[4,3-c]pyrazol-3-yl) (3,3-dimethylmorpholino) methanone (450 mg, 1 Eq, 822 μmol), dihydrofuran-2,5-dione (100 mg, 1.22 Eq, 999 μmol), DMAP (100 mg, 0.996 Eq, 819 μmol) and THF (5 mL). The reaction mixture was stirred at 60° C. for an additional 16 hours. The mixture was diluted with water (50 mL), extracted with EtOAc (50 mL×3), then the combined organic layers were washed with water (50 mL×2) and brine (50 mL), dried over anhydrous Na₂SO₄ and concentrated under reduced pressure. The crude product was purified by MPLC to provide 4-((5-(1-(3,5-difluorophenyl)-3-(3,3-dimethylmorpholine-4-carbonyl)-7-methoxy-1,4-dihydrochromeno[4,3-c]pyrazol-8-yl)-307-yridine-3-yl)amino)-4-oxobutanoic acid (340 mg, 0.47 mmol, 57%) as an off-white solid. [M+H]=648.3.

Synthesis of benzyl(S)-4-(2-((((9H-fluoren-9-yl) methoxy) carbonyl)amino)-3-aminopropanoyl) piperazine-1-carboxylate 2,2,2-trifluoroacetate: Into a 40-mL vial, was placed with a mixture of(S)-2-((((9H-fluoren-9-yl) methoxy) carbonyl)amino)-3-((tert-butoxycarbonyl)amino) propanoic acid (620 mg, 1 Eq, 1.45 mmol), HATU (660 mg, 1.19 Eq, 1.74 mmol), DIEA (570 mg, 768 μL, 3.03 Eq, 4.41 mmol) and DMF (6.5 mL). The reaction mixture was stirred at 20° C. for 10 minutes, then benzyl piperazine-1-carboxylate (385 mg, 1.20 Eq, 1.75 mmol)_{w} as added and the reaction mixture was stirred at 25° C. for an additional 1 hour. The mixture was diluted with water (50 mL), extracted with EtOAc (50 mL×3), then the combined organic layers were washed with water (50 mL×2) and brine (50 mL), dried over anhydrous Na₂SO₄ and concentrated under reduced pressure. The crude product was purified by MPLC to provide benzyl(S)-4-(2-((((9H-fluoren-9-yl) methoxy) carbonyl)amino)-3-((tert-butoxycarbonyl)amino) propanoyl) piperazine-1-carboxylate (760 mg, 1.1 mmol, 75%) as a yellow oil. [M+H]=629.3. Into a 8-mL vial, was placed a mixture of benzyl(S)-4-(2-((((9H-fluoren-9-yl) methoxy) carbonyl)amino)-3-((tert-butoxycarbonyl)amino) propanoyl) piperazine-1-carboxylate (760 mg, 1 Eq, 1.21 mmol) and DCM (8 mL), to which was added TFA (2.2 g, 1.5 mL, 16 Eq, 19 mmol). The reaction mixture was stirred at 25° C. for 1 hour then concentrated under reduced pressure. The crude product was purified by Prep-HPLC to provide benzyl(S)-4-(2-((((9H-fluoren-9-yl) methoxy) carbonyl)amino)-3-aminopropanoyl) piperazine-1-carboxylate 2,2,2-trifluoroacetate (635 mg, 0.87 mmol, 72%) as a white solid. [M+H]=529.3.

Step 6: Into a 40-mL vial, was placed a mixture of benzyl(S)-4-(2-((((9H-fluoren-9-yl) methoxy) carbonyl)amino)-3-aminopropanoyl) piperazine-1-carboxylate 2,2,2-trifluoroacetate (310 mg, 1.2 Eq, 482 μmol), DIEA (160 mg, 216 μL, 3.08 Eq, 1.24 mmol), HATU (466 mg, 3.05 Eq, 1.23 mmol) and DMF (3 mL). The reaction mixture was stirred at 20° C. for 10 minutes, then 4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanoic acid (310 mg, 1.19 Eq, 479 μmol)_{w} as added and the reaction mixture was stirred at 25° C. for an additional 1 hour. The mixture was diluted with 30 mL of water (30 mL), extracted with EtOAc (30 mL×3), the combined organic layers were washed with water (20 mL×2) and brine (30 mL), dried over anhydrous Na₂SO₄ and concentrated under reduced pressure. The crude product was purified by to provide benzyl(S)-4-(2-((((9H-fluoren-9-yl) methoxy) carbonyl)amino)-3-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido) propanoyl) piperazine-1-carboxylate (290 mg, 0.23 mmol, 56%) as a yellow oil. [M+H]=1158.4.

Step 7: Into a 8-mL vial, was placed a mixture of benzyl(S)-4-(2-((((9H-fluoren-9-yl) methoxy) carbonyl)amino)-3-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido) propanoyl) piperazine-1-carboxylate (290 mg, 1 Eq, 250 μmol) amd TFA (2.9 mL). The reaction mixture was stirred at 60° C. for 1 hour then concentrated under reduced pressure to afford (9H-fluoren-9-yl)methyl(S)-(3-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido)-1-oxo-1-(piperazin-1-yl) propan-2-yl) carbamate (165 mg, 0.13 mmol, 51%) as a brown oil, which was used directly in the next step without any purification. [M+H]=1024.3.

Step 8: Into a 8 mL vial, was placed a mixture of (9H-fluoren-9-yl)methyl(S)-(3-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido)-1-oxo-1-(piperazin-1-yl) propan-2-yl) carbamate (160 mg, 1 Eq, 156 μmol), sodium 2-bromoethane-1-sulfonate (70 mg, 2.1 Eq, 0.33 mmol), cesium carbonate (100 mg, 1.96 Eq, 307 μmol), potassium iodide (5.2 mg, 0.20 Eq, 31 μmol), and DMF (1.6 mL). The reaction mixture was stirred at 80° C. for 2 hours. The mixture was diluted with water (50 mL), extracted with EtOAc (30 mL×3), the combined organic layers were washed with water (20 mL×2), brine (30 mL), dried over anhydrous Na₂SO₄ and concentrated under reduced pressure to afford(S)-2-(4-(2-((((9H-fluoren-9-yl) methoxy) carbonyl)amino)-3-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido) propanoyl) piperazin-1-yl) ethane-1-sulfonic acid (130 mg, 80 μmol, 51%) as a light-brown oil, which was used directly in the next step without any purification. [M+H]=1132.2.

Step 9: Into a 8-mL vial, was placed a mixture of(S)-2-(4-(2-((((9H-fluoren-9-yl) methoxy) carbonyl)amino)-3-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido) propanoyl) piperazin-1-yl) ethane-1-sulfonic acid (130 mg, 1 Eq, 115 μmol) and DMF (1.5 mL), to which was added DBU (46 mg, 46 μL, 2.6 Eq, 0.30 mmol). The reaction mixture was stirred at 25° C. for 1 hour. The reaction mixture was diluted with water (20 mL), extracted with DCM (20 mL×3), dried over anhydrous Na₂SO₄, then concentrated under reduced pressure to afford(S)-2-(4-(2-amino-3-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido) propanoyl) piperazin-1-yl) ethane-1-sulfonic acid (60 mg, 46 μmol, 40%) as an off-white solid. [M+H]=910.2.

Step 10: (S)-2-(4-(2-amino-3-(4-((5-(1-(3,5-difluorophenyl)-3-(3,3-yl)amino)-4-oxobutanamido) propanoyl) piperazin-1-yl) ethane-1-sulfonic acid was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide(S)-2,2′,2″-(10-(2-((3-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido)-1-oxo-1-(4-(2-sulfoethyl) piperazin-1-yl) propan-2-yl)amino)-2-oxoethyl)-1,4,7,10-tetraazacyclododecane-1,4,7-triyl)triacetic acid--formic acid (1/1) (6.2 mg, 4.1 μmol, 4.7%) as a white solid. [M+H]=1296.5.

### Example 79: (R)-2,2′,2″-(10-(2-((6-((3-((5-(1-(3,5-difluorophenyl)-3-(3,3dimethylmorpholine-4-carbonyl)-7-methoxy-1,4-dihydrochromeno[4,3-c]pyrazol-8-yl) pyridin-3-yl)amino)-3-oxopropyl)amino)-6-oxo-5-(2-(4-(2-sulfoethyl) piperazin-1-yl)acetamido) hexyl)amino)-2-oxoethyl)-1,4,7,10-tetraazacyclododecane-1,4,7-triyl)triacetic acid--formic acid (1/1) (Compound 50)

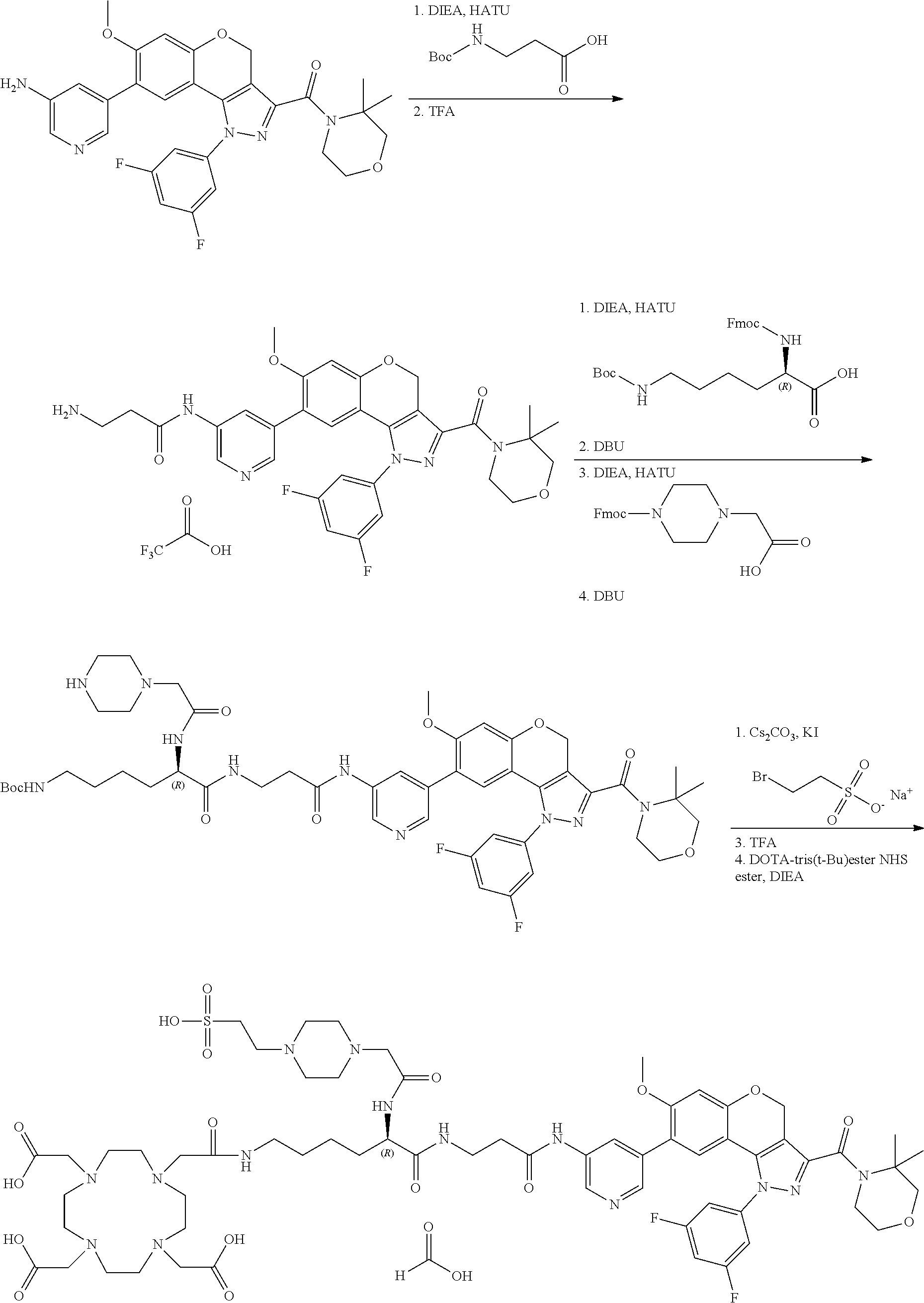

Step 1: (8-(5-aminopyridin-3-yl)-1-(3,5-difluorophenyl)-7-methoxy-1,4-dihydrochromeno[4,3-c]pyrazol-3-yl) (3,3-dimethylmorpholino) methanone (500 mg, 1 Eq, 913 μmol)_{w} as treated with 3-((tert-butoxycarbonyl)amino) propanoic acid (210 mg, 1.22 Eq, 1.11 mmol), DIEA and HATU in a manner similar to Step 6 of Example 78 to provide tert-butyl(3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl) carbamate (510 mg, 710 μmol, 77.7%) as an off-white solid. [M+H]=719.3.

Step 2: Into an 8-mL vial, was placed a mixture of tert-butyl(3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl) carbamate (510 mg, 1 Eq, 710 μmol) and DCM (5 mL), to which was added 2,2,2-trifluoroacetic acid (82 mg, 1.0 Eq, 0.72 mmol). The reaction mixture was stirred at 25° C. for 1 hour then concentrated under reduced pressure to afford 3-amino-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl) propanamide 2,2,2-trifluoroacetate (650 mg, 0.64 mmol, 90%) as a brown oil, which was used directly in the next step without any further purification. [M+H]=619.5.

Step 3: 3-amino-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl) propanamide 2,2,2-trifluoroacetate (650 mg, 1 Eq, 887 μmol)_{w} as treated with N₂-(((9H-fluoren-9-yl) methoxy) carbonyl)-N₆-(tert-butoxycarbonyl)-D-lysine (500 mg, 1.20 Eq, 1.07 mmol), DIEA and HATU to provide (9H-fluoren-9-yl)methyl tert-butyl(6-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-6-oxohexane-1,5-diyl) (R)-dicarbamate (450 mg, 0.29 mmol, 33%) as an off-white solid. [M+H]=1069.5.

Step 4: (9H-fluoren-9-yl)methyl tert-butyl(6-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-6-oxohexane-1,5-diyl) (R)-dicarbamate (450 mg, 1 Eq, 421 μmol) is treated with DBU in a manner similar to Step 9 or Example 78 to provide tert-butyl(R)-(5-amino-6-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-6-oxohexyl) carbamate (400 mg, 0.38 mmol, 90%) as an off-white solid. [M+H]=847.3.

Step 5: tert-butyl(R)-(5-amino-6-((3-((5-(1-(3,5-difluorophenyl)-3-(3,3-yl)amino)-3-oxopropyl)amino)-6-oxohexyl) carbamate (400 mg, 1 Eq, 472 μmol)_{w} as treated with 2-(4-(((9H-fluoren-9-yl) methoxy) carbonyl) piperazin-1-yl)acetic acid (210 mg, 1.21 Eq, 573 μmol), DIEA and HATU to provide (9H-fluoren-9-yl)methyl (R)-4-(2-((6-((tert-butoxycarbonyl)amino)-1-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-1-oxohexan-2-yl)amino)-2-oxoethyl) piperazine-1-carboxylate (315 mg, 264 μmol, 55.8%) as an off-white solid. [M+H]=1195.5.

Step 6: (9H-fluoren-9-yl)methyl (R)-4-(2-((6-((tert-butoxycarbonyl)amino)-1-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-1-oxohexan-2-yl)amino)-2-oxoethyl) piperazine-1-carboxylate was treated with DBU in a manner similar to Step 9 or Example 78 to provide tert-butyl(R)-(6-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-6-oxo-5-(2-(piperazin-1-yl)acetamido) hexyl) carbamate (250 mg, 0.21 mmol, 78%) as an off-white solid. [M+H]=847.3.

Step 7: tert-butyl(R)-(6-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-6-oxo-5-(2-(piperazin-1-yl)acetamido) hexyl) carbamate was treated with sodium 2-bromoethane-1-sulfonate in a manner similar to Step 8 of Example 78 to provide (R)-2-(4-(2-((6-((tert-butoxycarbonyl)amino)-1-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-1-oxohexan-2-yl)amino)-2-oxoethyl) piperazin-1-yl) ethane-1-sulfonic acid (180 mg, 0.15 mmol, 58%) as a yellow oil. [M+H]=1081.4.

Step 8: Into a 8-mL vial, was placed a mixture of (R)-2-(4-(2-((6-((tert-butoxycarbonyl)amino)-1-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-1-oxohexan-2-yl)amino)-2-oxoethyl) piperazin-1-yl) ethane-1-sulfonic acid (180 mg, 1 Eq, 166 μmol) and DCM (2 mL), to which was added TFA (0.6 g, 0.4 mL, 3e+1 Eq, 5 mmol). The reaction mixture was stirred at 25° C. for 2 hours then concentrated under reduced pressure to afford 2,2,2-trifluoroacetic acid--(R)-2-(4-(2-((6-amino-1-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-1-oxohexan-2-yl)amino)-2-oxoethyl) piperazin-1-yl) ethane-1-sulfonic acid (1/1) (210 mg, 0.15 mmol, 92%) as a light yellow solid, which was used directly in the next step without any purification. [M+H]=981.3, [M−H]=979.3.

Step 9: 2,2,2-trifluoroacetic acid--(R)-2-(4-(2-((6-amino-1-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-1-oxohexan-2-yl)amino)-2-oxoethyl) piperazin-1-yl) ethane-1-sulfonic acid (1/1)_{w} as treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide (R)-2,2′,2″-(10-(2-((6-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropyl)amino)-6-oxo-5-(2-(4-(2-sulfoethyl) piperazin-1-yl)acetamido) hexyl)amino)-2-oxoethyl)-1,4,7,10-tetraazacyclododecane-1,4,7-triyl)triacetic acid--formic acid (1/1) (18.1 mg, 11.5 μmol, 12.6%) as a white solid. Calc'd for C₆₃H₈₆F2N₁₄O₁₉S: 1412.59, found [M+H−FA]⁺: 1368.4.

### Example 80:2,2′,2″-(10-((8R,11R,14R)-14-carbamoyl-8,11-bis(carboxymethyl)-23-((3-(1(3,5-dichlorophenyl)-3-(3,3-dimethylmorpholine-4-carbonyl)-7-methoxy-1,4-dihydrochromeno[4,3-c]pyrazol-8-yl)phenyl)amino)-2,6,9,12,20,23-hexaoxo-3,7,10,13,19-pentaazatricosyl)-1,4,7,10-tetraazacyclododecane-1,4,7-triyl)triacetic acid (Compound 51)

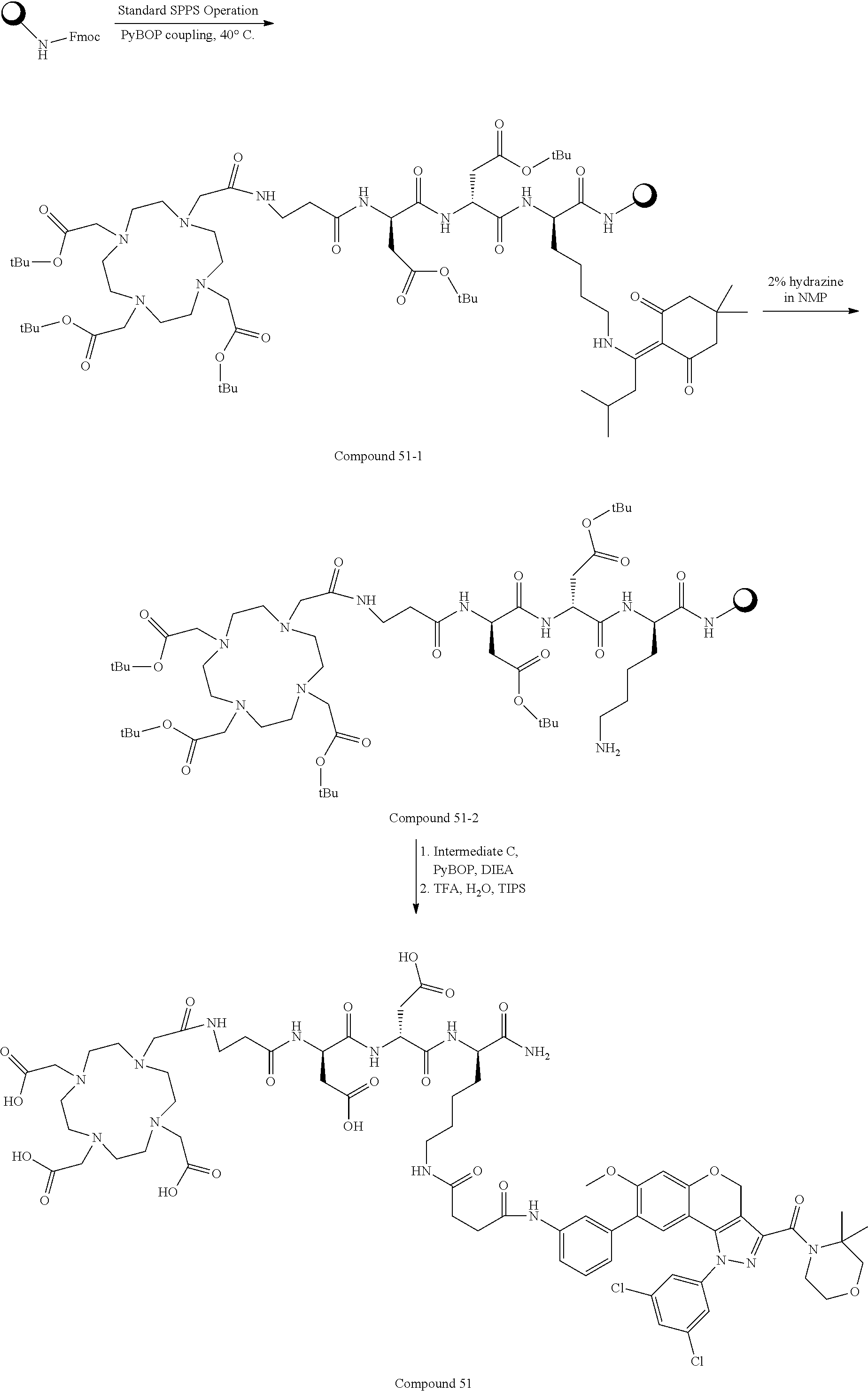

Step 1: Synthesis of Compound 51-1: The automated SPPS was executed with a Syro II peptide synthesizer (Biotage). Resin pre-swelling: the Rink amide resin (100 mg/tube, 100-200 mesh, 0.62 mmol/g, total 5 tubes)_{w} as swelled in NMP (1 mL/tube) for 15 min at room temperature under a nitrogen atmosphere. The resin was then washed 4 times with NMP (1 mL/tube). Parameter: incubation time: 15 min, vortex time: 15 s, break time: 1 min, temperature: room temperature, empty time: 1 min. SPPS coupling cycle: A) Fmoc de-protection: the resin (100 mg/tube, total 5 tubes)_{w} as treated with 20% of piperidine in NMP (1 mL) for 20 min at room temperature under a nitrogen atmosphere. The resin was then washed 4 times with NMP (1 mL/tube). Parameter: reaction time: 20 min, vortex time: 15 s, break time: 1 min, temperature: room temperature, empty time: 1 min. B) PyBOP coupling: the resin (100 mg/tube, total 5 tubes) was treated with the mixture of amino acid or DOTA-(OfBu) 3 (4 eq.), PyBOP (4 eq.) and DIPEA (8 eq.) in NMP (1 mL) for 45 min at 40° C. under a nitrogen atmosphere. Then, the resin was washed 4 times with NMP (1 mL/tube). Parameter: reaction time: 45 min, vortex time: 15 s, break time: 3 min, temperature: 40° C., empty time: 1 min.

Step 2. Synthesis of Compound 51-2: Compound 51-1 on resin (500 mg)_{w} as swelled with NMP (5 mL) for 15 min at room temperature. The solvent was then removed. To the resin (500 mg) in a 25 mL reactor was added 2% hydrazine in NMP (5 mL) at room temperature under a nitrogen atmosphere. The mixture was bubbled for 2 h and filtered, washed with NMP (5 mL×8). The reaction was monitored by LCMS.

Step 3: To the resin (500 mg) in 25 mL reactor were added Intermediate C (2 eq.), PyBOP (2 eq.) and DIPEA (4 eq.) in NMP (4 mL). After the reaction mixture was incubated for 2 h at 40° C., the solvent was removed and the resin was washed with NMP (5 mL×6).

Step 4: Synthesis of Compound 51: The resin was treated with the cleavage solution (v v v TFA/H₂O/TIS=37:1:1) for 2 h at room temperature. After filtration, the filtrate was concentrated under reduced pressure. The residue was precipitated with cold ether. After centrifugation, the solid was collected and purified by prep-HPLC. The desired fractions were combined and lyophilized to afford Compound 51 as a white solid (35.3 mg, 7.35%). Calc'd for C₆₇H₈₆C₁₂N₁₄O₂₁:1492.5, found [M+2H]²⁺: 748.2.

### Example 81:2,2′,2″-(10-(2-((2-(2-(3-((5-(1-(3,5-dichlorophenyl)-3-(3,3-dimethylmorpholine-4-carbonyl)-7-methoxy-1,4-dihydrochromeno|4,3-c|pyrazol-8-yl) pyridin-3-yl)amino)-3-oxopropoxy)ethoxy)ethyl)amino)-2-oxoethyl)-1,4,7,10-tetraazacyclododecane-1,4,7-triyl)triacetic acid--2,2,2-trifluoroacetic acid (1/1) (Compound 52)

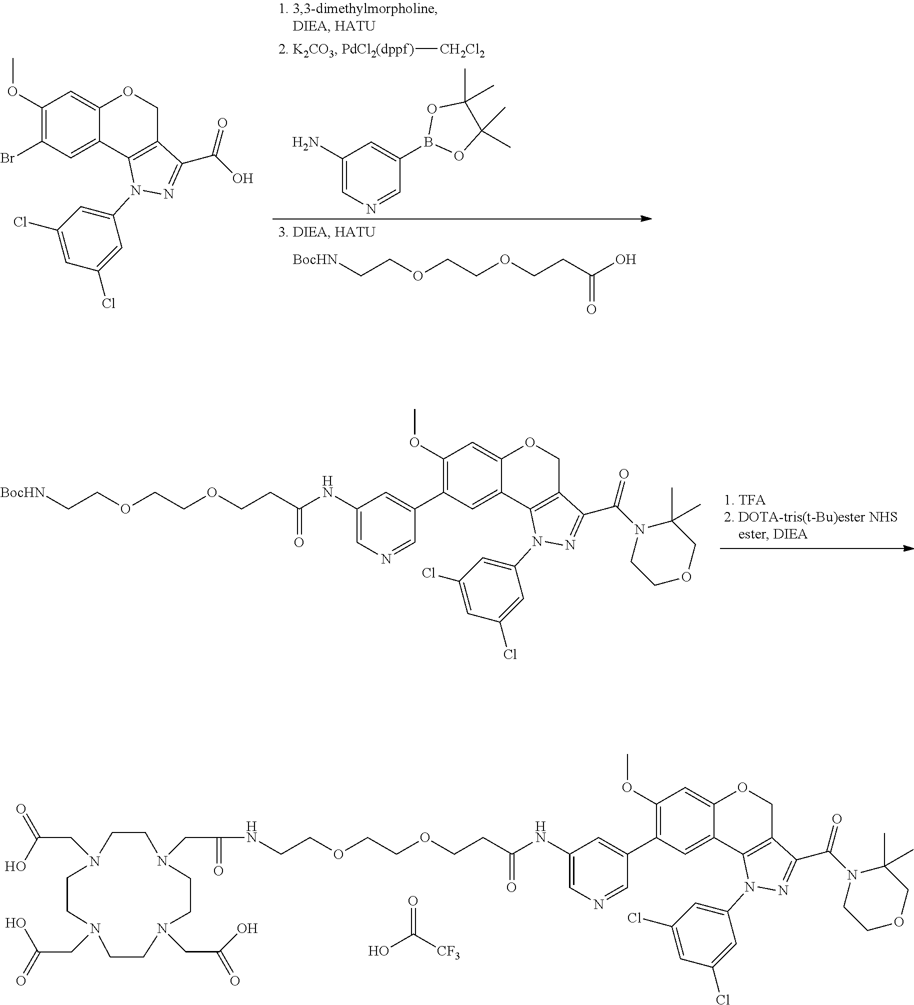

Step 1: 8-bromo-1-(3,5-dichlorophenyl)-7-methoxy-1,4-dihydrochromeno[4,3-c]pyrazole-3-carboxylic acid was treated with 3,3-dimethylmorpholine, DIEA, and HATU in a manner similar to Example 78, Step 3 to provide (8-bromo-1-(3,5-dichlorophenyl)-7-methoxy-1,4-dihydrochromeno[4,3-c]pyrazol-3-yl) (3,3-dimethylmorpholino) methanone as an off-white solid. [M+H]=566.0, 568.0.

Step 2: (8-bromo-1-(3,5-dichlorophenyl)-7-methoxy-1,4-dihydrochromeno[4,3-c]pyrazol-3-yl) (3,3-dimethylmorpholino) methanone was treated with 5-(4,4,5,5-tetramethyl-1,3,2-dioxaborolan-2-yl) pyridin-3-amine, 1, l′-Bis(di-t-butylphosphino) ferrocene palladium dichloride, and K₂CO₃ in a manner similar to Example 1, Step 6 to provide (8-(5-aminopyridin-3-yl)-1-(3,5-dichlorophenyl)-7-methoxy-1,4-dihydrochromeno[4,3-c]pyrazol-3-yl) (3,3-dimethylmorpholino) methanone (300 mg, 0.47 mmol, 88%) as an off-white solid. [M+H]=580.2, 582.2.

Step 3: (8-(5-aminopyridin-3-yl)-1-(3,5-dichlorophenyl)-7-methoxy-1,4-dihydrochromeno[4,3-c]pyrazol-3-yl) (3,3-dimethylmorpholino) methanone was treated with 2,2-dimethyl-4-oxo-3,8,11-trioxa-5-azatetradecan-14-oic acid, DIEA and HATU in a manner similar to Example 78, Step 6 to provide tert-butyl(2-(2-(3-((5-(1-(3,5-dichlorophenyl)-3-(3,3-yl)amino)-3-oxopropoxy)ethoxy)ethyl) carbamate as an off-white solid. [M+H]=839.2, 841.2.

Step 4: tert-butyl(2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl) carbamate was treated with TFA in a manner similar to Example 79, Step 8 to provide 3-(2-(2-aminoethoxy)ethoxy)-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) propanamide 2,2,2-trifluoroacetate (100 mg, 0.11 mmol, 89%) as a light yellow oil. [M+H−TFA]=739.2, 741.2.

Step 5. 3-(2-(2-Aminoethoxy)ethoxy)-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) propanamide 2,2,2-trifluoroacetate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(2-((2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl)amino)-2-oxoethyl)-1,4,7,10-tetraazacyclododecane-1,4,7-triyl)triacetic acid--2,2,2-trifluoroacetic acid (1/1) as a white solid. Calc'd for C₅₄H₆₇C₁₂F3N₁₀O₁₆:1238.41, found [M+H−TFA]⁺: 1125.5, 1127.5.

### Example 82:2,2′,2″-(10-(2-((2-(3-((5-(1-(3,5-dichlorophenyl)-3-(3,3-dimethylmorpholine-4carbonyl)-7-methoxy-1,4-dihydrochromeno|4,3-c|pyrazol-8-yl) pyridin-3-yl)amino)-3-oxopropoxy)ethyl)amino)-2-oxoethyl)-1,4,7,10-tetraazacyclododecane-1,4,7-triyl)triacetic acid--2,2,2-trifluoroacetic acid (Compound 53)

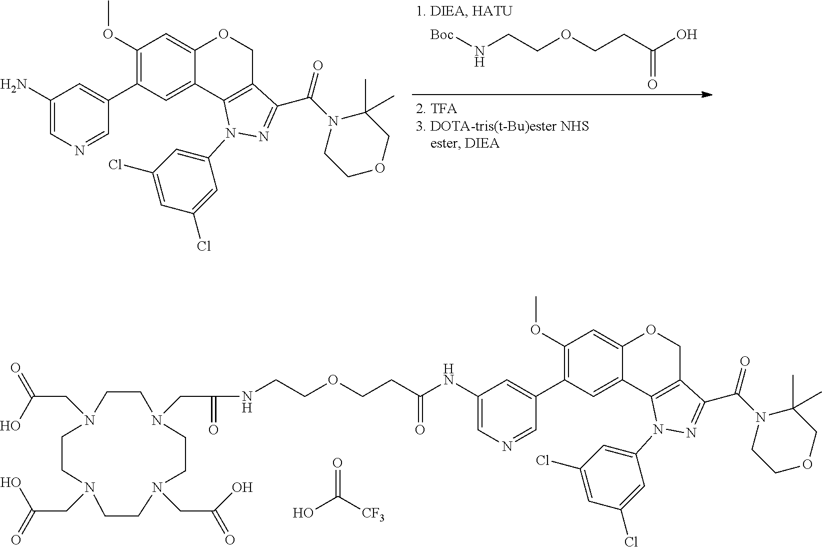 [SMILES: COc1cc2c(cc1-c1cncc(N)c1)-c1c(c(C(=O)N3CCOCC3(C)C)nn1-c1cc(Cl)cc(Cl)c1)CO2 ; CC(C)(C)OC(=O)NCCOCCC(=O)O ; COc1cc2c(cc1-c1cncc(NC(=O)CCOCCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2 ; O=C(O)C(F)(F)F]

Step 1: (8-(5-aminopyridin-3-yl)-1-(3,5-dichlorophenyl)-7-methoxy-1,4-dihydrochromeno[4,3-c]pyrazol-3-yl) (3,3-dimethylmorpholino) methanone was treated with 3-(2-((tert-butoxycarbonyl)amino) ethoxy) propanoic acid, DIEA and HATU in a manner similar to Example 78, Step 6 to provide tert-butyl(2-(3-((5-(1-(3,5-dichlorophenyl)-3-(3,3-yl)amino)-3-oxopropoxy)ethyl) carbamate as an off-white solid. [M+H]=795.3, 797.3.

Step 2: tert-butyl(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethyl) carbamate was treated with TFA in a manner similar to Example 79, Step 8 to provide 3-(2-aminoethoxy)-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) propanamide 2,2,2-trifluoroacetate as a light yellow crude oil. [M+H−TFA]=695.2, 697.2.

Step 3: 3-(2-Aminoethoxy)-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) propanamide 2,2,2-trifluoroacetate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(2-((2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethyl)amino)-2-oxoethyl)-1,4,7,10-tetraazacyclododecane-1,4,7-triyl)triacetic acid--2,2,2-trifluoroacetic acid (1/1) (63.9 mg, 53.4 μmol, 51%) as a white solid. Calc'd for C₅₂H₆₃C₁₂F3N₁₀O₁₅:1194.38, found [M+H−TFA]⁺: 1081.4, 1083.4.

### Example 83:2,2′,2″-(10-(2-((((2S,3R,4R,5R,6S)-6-((2-(2-(3-((5-(1-(3,5-dichlorophenyl)-3(3,3-dimethylmorpholine-4-carbonyl)-7-methoxy-1,4-dihydrochromeno|4,3-c]pyrazol-8-yl) pyridin-3-yl)amino)-3-oxopropoxy)ethoxy)ethyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid--2,2,2-trifluoroacetic acid (1/1) (Compound 54)

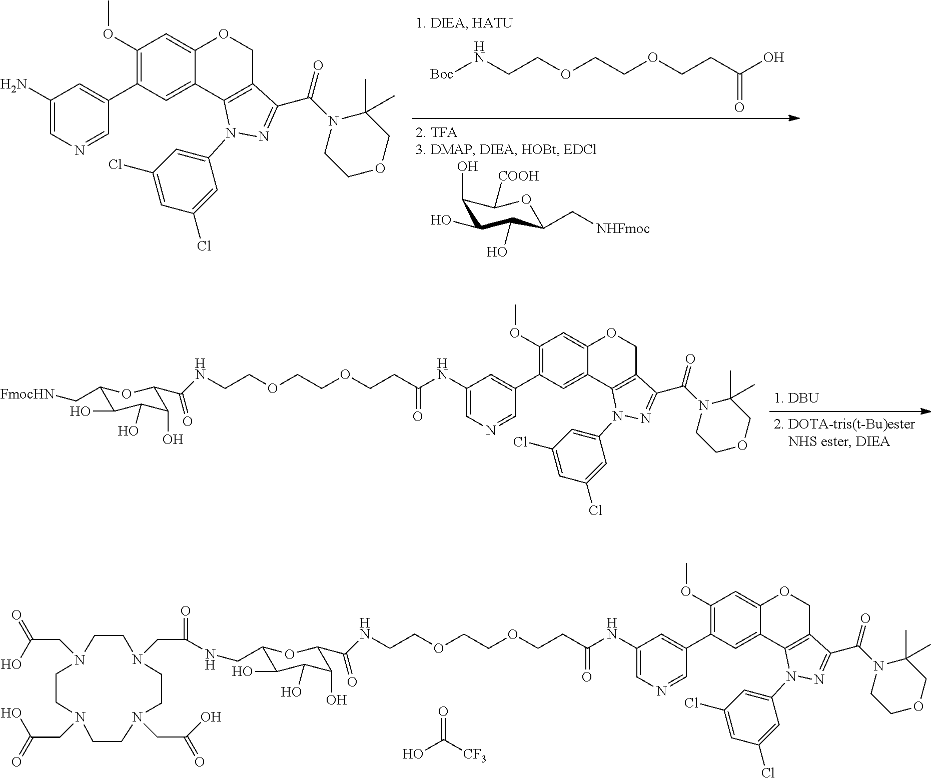

Step 1: (8-(5-aminopyridin-3-yl)-1-(3,5-dichlorophenyl)-7-methoxy-1,4-dihydrochromeno[4,3-c]pyrazol-3-yl) (3,3-dimethylmorpholino) methanone was treated with 2,2-dimethyl-4-oxo-3,8, 11-trioxa-5-azatetradecan-14-oic acid, HATU, and DIEA in a manner similar to Example 78, Step 6 to provide tert-butyl(2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl) carbamate as an off-white solid.

Step 2: tert-butyl(2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl) carbamate was treated with TFA in a manner similar to Example 79, Step 8 to provide 3-(2-(2-aminoethoxy)ethoxy)-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) propanamide--2,2,2-trifluoroacetaldehyde (1/1) (210 mg, 0.19 mmol, 82%) as a light yellow crude oil, which was used directly in the next step without any purification. [M+H−TFA]=739.5, 741.5.

Step 3: Into a 8-mL vial, was placed a mixture of 3-(2-(2-aminoethoxy)ethoxy)-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) propanamide--2,2,2-trifluoroacetaldehyde (1/1) (100 mg, 75% Wt, 1 Eq, 89.5 μmol), (2S,3R,4R,5R,6S)-6-(((((9H-fluoren-9-yl) methoxy) carbonyl)amino)methyl)-3,4,5-trihydroxytetrahydro-2H-pyran-2-carboxylic acid (115 mg, 2.99 Eq, 268 μmol), 3-(((ethylimino)methylene)amino)-N,N-dimethylpropan-1-amine hydrochloride (25 mg, 1.5 Eq, 0.13 mmol), 1H-benzo[d][1,2,3]triazol-1-ol hydrate (20 mg, 1.5 Eq, 0.13 mmol), N-ethyl-N-isopropylpropan-2-amine (35 mg, 3.0 Eq, 0.27 mmol), N,N-dimethylpyridin-4-amine (10 mg, 0.91 Eq, 82 μmol) and DMF (1 mL). The reaction mixture was stirred at 27° C. for 3 hours. The mixture was directly purified by MPLC to afford (9H-fluoren-9-yl)methyl (((2S,3R,4R,5R,6S)-6-((2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl) carbamoyl)-3,4,5-trihydroxytetrahydro-2H-pyran-2-yl)methyl) carbamate (57 mg, 50 μmol, 55%) as a yellow solid. [M+H]=1150.8, 1152.8.

Step 4: (9H-fluoren-9-yl)methyl (((2S,3R,4R,5R,6S)-6-((2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl) carbamoyl)-3,4,5-trihydroxytetrahydro-2H-pyran-2-yl)methyl) carbamate was treated with DBU in a manner similar to Example 78, Step 9 to provide (2S,3R,4R,5R,6S)-6-(aminomethyl)-N-(2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl)-3,4,5-trihydroxytetrahydro-2H-pyran-2-carboxamide (57 mg, 42 μmol, 84%) as a brown oil, which was used directly in the next step without any purification. [M+H]=928.6, 930.6.

Step 5. (2S,3R,4R,5R,6S)-6-(aminomethyl)-N-(2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl)-3,4,5-trihydroxytetrahydro-2H-pyran-2-carboxamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(2-((((2S,3R,4R,5R,6S)-6-((2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid--2,2,2-trifluoroacetic acid (1/1) (40.1 mg, 28.1 μmol, 67%) as an off-white solid. Calc'd for C₆₁H₇₈C₁₂F3N₁₁O₂₁:1427.47, found [M+H−TFA]⁺: 1314.6, 1316.6.

### Example 84: (R)-2,2′,2″-(10-(2-((2-((1-carboxy-4-((2-((5-(1-(3,5-dichlorophenyl)-3-(3,3dimethylmorpholine-4-carbonyl)-7-methoxy-1,4-dihydrochromeno|4,3-c]pyrazol-8-yl) pyridin-3-yl)amino)-2-oxoethyl)amino)-4-oxobutyl)amino)-2-oxoethyl)amino)-2-oxoethyl)-1,4,7,10-tetraazacyclododecane-1,4,7-triyl)triacetic acid--2,2,2-trifluoroacetic acid (1/1) (Compound 55)

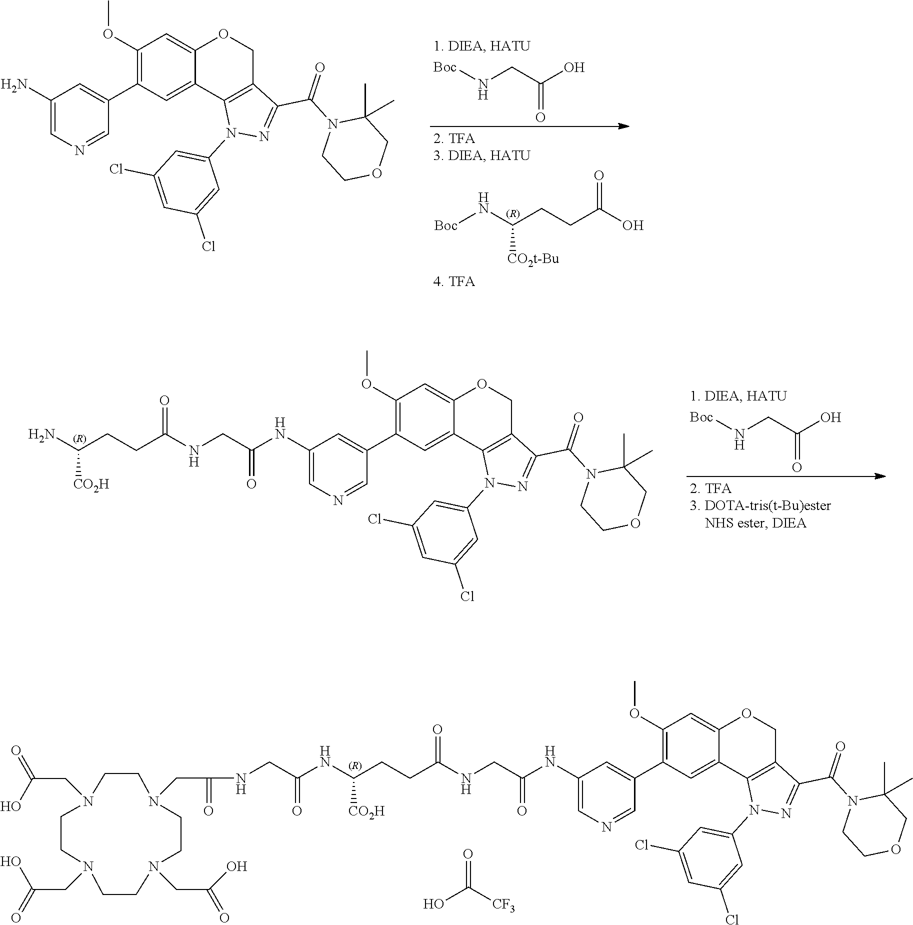

Step 1: (8-(5-aminopyridin-3-yl)-1-(3,5-dichlorophenyl)-7-methoxy-1,4-dihydrochromeno[4,3-c]pyrazol-3-yl) (3,3-dimethylmorpholino) methanone was treated with (tert-butoxycarbonyl)glycine, DIEA, and HATU in a manner similar to Example 78, Step 6 to provide tert-butyl(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl) carbamate (580 mg, 0.71 mmol, 91%) as a yellow solid. [M+H]=737.3.

Step 2: tert-Butyl (2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl) carbamate was treated with TFA in a manner similar to Example 79, Step 8 to provide 2-amino-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)acetamide 2,2,2-trifluoroacetate (520 mg, 0.55 mmol, 78%) as a brown solid, which was used directly for next step without any purification. [M+H−TFA]=637.3.

Step 3: 2-Amino-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)acetamide 2,2,2-trifluoroacetate was treated with (R)-5-(tert-butoxy)-4-((tert-butoxycarbonyl)amino)-5-oxopentanoic acid, DIEA, and HATU in a manner similar to Example 78, Step 6 to provide tert-butyl N₂-(tert-butoxycarbonyl)-N₅-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-D-glutaminate (200 mg, 217 μmol, 84.8%) as a yellow solid. [M+H]=922.5, 924.5.

Step 4: tert-Butyl N₂-(tert-butoxycarbonyl)-N₅-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-D-glutaminate was treated with TFA in a manner similar to Example 79, Step 8 to provide tert-butyl N₅-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-D-glutaminate 2,2,2-trifluoroacetate (160 mg, 171 μmol, 78.8%) as a brown solid, which was used directly in the next step without any purification. [M+H−TFA]=822.5, 824.5.

Step 5: tert-Butyl N₅-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-D-glutaminate 2,2,2-trifluoroacetate was treated with (tert-butoxycarbonyl)glycine, DIEA, and HATU in a manner similar to Example 78, Step 6 to provide tert-butyl N₂-((tert-butoxycarbonyl)glycyl)-N₅-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-D-glutaminate (60 mg, 61 μmol, 36%) as a yellow solid. [M+H]=979.4, 981.4.

Step 6: tert-Butyl N₂-((tert-butoxycarbonyl)glycyl)-N₅-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-D-glutaminate was treated with TFA in a manner similar to Example 79, Step 8 to provide N₅-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-N₂-glycyl-D-glutamine--2,2,2-trifluoroacetic acid (1/1) (70 mg, 52 μmol, 85%) as a brown solid, which was used directly for next step without any purification. [M+H−TFA]=823.2, 825.2.

Step 7. N₅-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-N₂-glycyl-D-glutamine--2,2,2-trifluoroacetic acid (1/1)_{w} as treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide (R)-2,2′,2″-(10-(2-((2-((1-carboxy-4-((2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)amino)-4-oxobutyl)amino)-2-oxoethyl)amino)-2-oxoethyl)-1,4,7,10-tetraazacyclododecane-1,4,7-triyl)triacetic acid--2,2,2-trifluoroacetic acid (1/1) (18.7 mg, 14.1 μmol, 27%) as a white solid. MS: Calc'd for C₅₆H₆₇C₁₂F3N₁₂O₁₈:1322.40, found [M+H−TFA]⁺: 1209.6, 1211.6.

### Example 85:2,2′,2″-(10-(2-(((S)-1-((2-((5-(1-(3,5-dichlorophenyl)-3-(3,3dimethylmorpholine-4-carbonyl)-7-methoxy-1,4-dihydrochromeno|4,3-c]pyrazol-8-yl) pyridin-3-yl)amino)-2-oxoethyl)amino)-1,5-dioxo-5-(((2S,3R,4R,5R)-2,3,4,5,6-pentahydroxyhexyl)amino) pentan-2-yl)amino)-2-oxoethyl)-1,4,7,10-tetraazacyclododecane-1,4,7-triyl)triacetic acid--2,2,2-trifluoroacetic acid (1/1) (Compound 56)

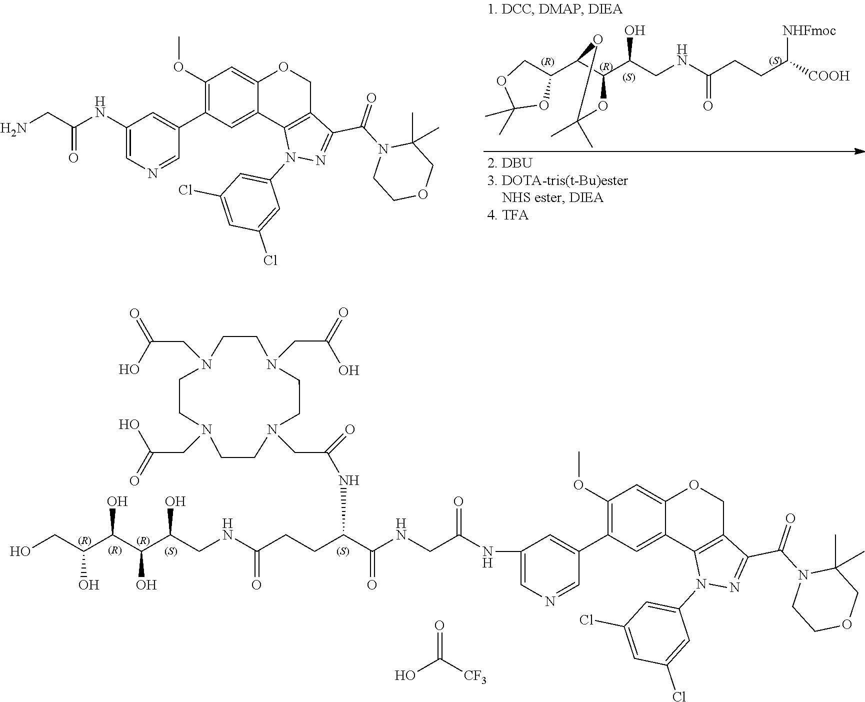

Step 1: Into a 8-mL vial, was placed a mixture of N₂-(((9H-fluoren-9-yl) methoxy) carbonyl)-N₅-((2S)-2-hydroxy-2-((4′R,5R)-2,2,2′,2′-tetramethyl-[4,4′-bi (1,3-dioxolan)]-5-yl)ethyl)-L-glutamine (70 mg, 1.5 Eq, 0.11 mmol), DCC (30 mg, 1.9 Eq, 0.15 mmol), N,N-dimethylpyridin-4-amine (30 mg, 3.1 Eq, 0.25 mmol), DIEA (30 mg, 40 μL, 3.0 Eq, 0.23 mmol) and DCM (1 mL). The reaction mixture was stirred at 20° C. for 10 minutes, then 2-amino-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)acetamide (50 mg, 1 Eq, 78 μmol)_{w} as added and the reaction mixture was stirred at 25° C. for additional 1 hour. The mixture was directly purified by MPLC to provide (9H-fluoren-9-yl)methyl ((2S)-1-((2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)amino)-5-(((2S)-2-hydroxy-2-((4′R,5R)-2,2,2′,2′-tetramethyl-[4,4′-bi (1,3-dioxolan)]-5-yl)ethyl)amino)-1,5-dioxopentan-2-yl) carbamate (60 mg, 49 μmol, 62%) as a white solid. [M+H]=1233.3, 1235.3.

Step 2: (9H-Fluoren-9-yl)methyl ((2S)-1-((2-((5-(1-(3,5-dichlorophenyl)-3-(3,3-yl)amino)-2-oxoethyl)amino)-5-(((2S)-2-hydroxy-2-((4′R,5R)-2,2,2′,2′-tetramethyl-[4,4′-bi (1,3-dioxolan)]-5-yl)ethyl)amino)-1,5-dioxopentan-2-yl) carbamate was treated with DBU in a manner similar to Example 78, Step 9 to provide (2S)-2-amino-N₁-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-N₅-((2S)-2-hydroxy-2-((4′R,5R)-2,2,2′,2′-tetramethyl-[4,4′-bi (1,3-dioxolan)]-5-yl)ethyl) pentanediamide (40 mg, 40 μmol, 81%) as a white solid. [M+H]=1011.1, 1013.1.

Step 3: (2S)-2-amino-N₁-(2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-N₅-((2S)-2-hydroxy-2-((4′R,5R)-2,2,2′,2′-tetramethyl-[4,4′-bi (1,3-dioxolan)]-5-yl)ethyl) pentanediamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(2-(((2S)-1-((2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)amino)-5-(((2S)-2-hydroxy-2-((4′R,5R)-2,2,2′,2′-tetramethyl-[4,4′-bi (1,3-dioxolan)]-5-yl)ethyl)amino)-1,5-dioxopentan-2-yl)amino)-2-oxoethyl)-1,4,7,10-tetraazacyclododecane-1,4,7-triyl)triacetic acid (36 mg, 26 μmol, 65%) as a white solid. [M+H−56]=1395.9, 1397.9.

Step 4: 2,2′,2″-(10-(2-(((2S)-1-((2-((5-(1-(3,5-Dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)amino)-5-(((2S)-2-hydroxy-2-((4′R,5R)-2,2,2′,2′-tetramethyl-[4,4′-bi (1,3-dioxolan)]-5-yl)ethyl)amino)-1,5-dioxopentan-2-yl)amino)-2-oxoethyl)-1,4,7,10-tetraazacyclododecane-1,4,7-triyl)triacetic acid is treated with TFA in a manner similar to Example 79, Step 8 to provide 2,2′,2″-(10-(2-(((S)-1-((2-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)amino)-1,5-dioxo-5-(((2S,3R,4R,5R)-2,3,4,5,6-pentahydroxyhexyl)amino) pentan-2-yl)amino)-2-oxoethyl)-1,4,7,10-tetraazacyclododecane-1,4,7-triyl)triacetic acid--2,2,2-trifluoroacetic acid (1/1) (7.0 mg, 4.9 μmol, 23%) as a white solid. Calc'd for C₆₀H₇₇C₁₂F3N₁₂P₂₁:1428.47, found [M+H−TFA]⁺: 1315.6, 1317.6.

### Example 86:2,2′,2″-(10-(2-((2-(3-((4-((5-(1-(3,5-difluorophenyl)-3-(3,3-dimethyl-morpholine-4-carbonyl)-7-methoxy-1,4- dihydrochromeno|4,3-c]pyrazol-8-yl) pyridin-3-yl)amino)-4-oxobutyl)amino)-3-oxopropoxy)ethyl)amino)-2-oxoethyl)-1,4,7,10-tetraazacyclododecane-1,4,7-triyl)triacetic acid-2,2,2-trifluoroacetic acid (1/1) (Compound

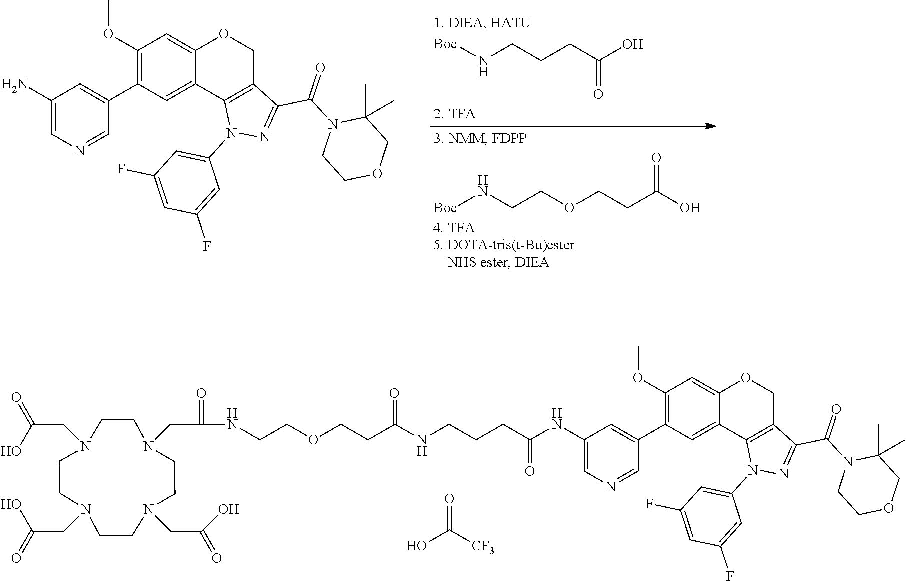 [SMILES: COc1cc2c(cc1-c1cncc(N)c1)-c1c(c(C(=O)N3CCOCC3(C)C)nn1-c1cc(F)cc(F)c1)CO2 ; CC(C)(C)OC(=O)NCCCC(=O)O ; CC(C)(C)OC(=O)NCCOCCC(=O)O ; COc1cc2c(cc1-c1cncc(NC(=O)CCCNC(=O)CCOCCNC(=O)CN3CCN(CC(=O)O)CCN(CC(=O)O)CCN(CC(=O)O)CC3)c1)-c1c(c(C(=O)N3CCOCC3(C)C)nn1-c1cc(F)cc(F)c1)CO2 ; O=C(O)C(F)(F)F]

Step 1: (8-(5-Aminopyridin-3-yl)-1-(3,5-difluorophenyl)-7-methoxy-1,4-dihydrochromeno[4,3-c]pyrazol-3-yl) (3,3-dimethylmorpholino) methanone was treated with 4-((tert-butoxycarbonyl)amino) butanoic acid, HATU, and DIEA in a manner similar to tert-butyl (4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl) carbamate (450 mg, 614 μmol, 74.7%) as a yellow oil. [M+H]=733.3.

Step 2: tert-Butyl (4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl) carbamate is treated with TFA in a manner similar to Example 78, Step 8 to provide 4-amino-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl) butanamide 2,2,2-trifluoroacetate (450 mg, 603 μmol, 98.1%) as a yellow oil, which was used in the next step without further purification. [M+H]=633.3.

Step 3: Into a 8-mL vial was placed a mixture of 3-(2-((tert-butoxycarbonyl)amino) ethoxy) propanoic acid (80 mg, 1.2 Eq, 0.34 mmol), pentafluorophenyldiphenylphosphinate (140 mg, 1.24 Eq, 364 μmol), 4-methylmorpholine (90 mg, 98 μL, 3.0 Eq, 0.89 mmol), 4-amino-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl) butanamide 2,2,2-trifluoroacetate (220 mg, 1 Eq, 295 μmol) and DMF (5 mL). The reaction mixture was stirred at 25° C. for 1 hour. The mixture was directly purified by MPLC to provide tert-butyl(2-(3-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-3-oxopropoxy)ethyl) carbamate (85 mg, 0.10 mmol, 34%) as a yellow solid. [M+H]=848.5.

Step 4: tert-Butyl (2-(3-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-3-oxopropoxy)ethyl) carbamate was treated with TFA in a manner similar to Example 79, Step 8 to provide 4-(3-(2-aminoethoxy) propanamido)-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl) butanamide (80 mg, 0.11 mmol, 110%) as a yellow oil, which was used in the next step without further purification.

Step 5. 4-(3-(2-aminoethoxy) propanamido)-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl) butanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(2-((2-(3-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-3-oxopropoxy)ethyl)amino)-2-oxoethyl)-1,4,7,10-tetraazacyclododecane-1,4,7-triyl)triacetic acid--2,2,2-trifluoroacetic acid (1/1) (46.8 mg, 37.5 μmol, 40%) as a white solid. MS: Calc'd for C₅₆H₇₀F5N₁₁O₁₆:1247.49, found [M+H−TFA]: 1135.6.

### Example 87: (R)-2,2′,2″-(10-(2-((5-amino-6-((4-((5-(1-(3,5-difluorophenyl)-3-(3,3dimethylmorpholine-4-carbonyl)-7-methoxy-1,4-dihydrochromeno|4,3-c]pyrazol-8-yl) pyridin-3-yl)amino)-4-oxobutyl)amino)-6-oxohexyl)amino)-2-oxoethyl)-1,4,7,10-tetraazacyclododecane-1,4,7-triyl)triacetic acid--2,2,2-trifluoroacetic acid (1/1) (Compound 58)

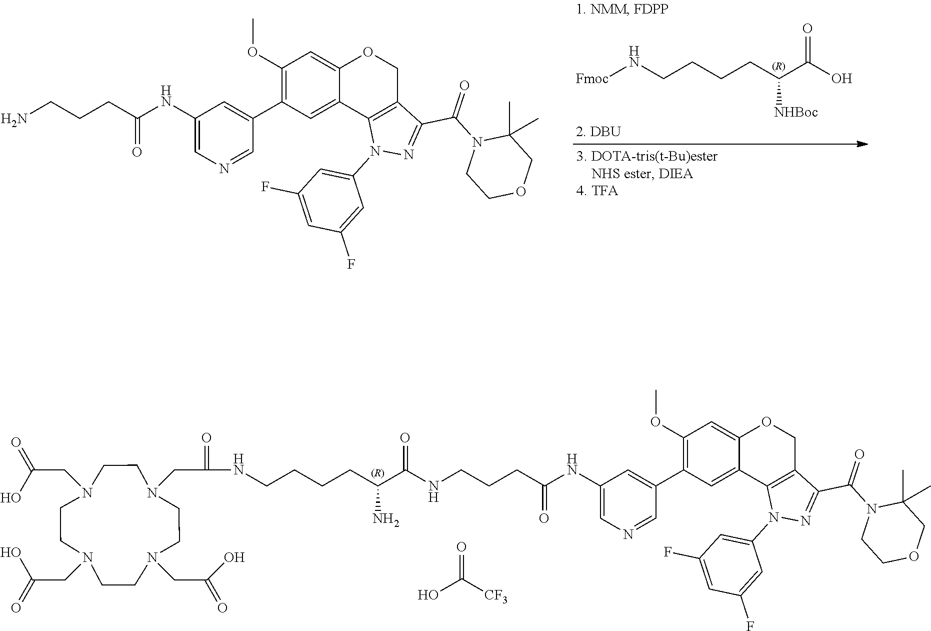

Step 1: 4-amino-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl) butanamide (220 mg, 1 Eq, 348 μmol)_{w} as treated with N₆-(((9H-fluoren-9-yl) methoxy) carbonyl)-N₂-(tert-butoxycarbonyl)-D-lysine, NMM, and FDPP in a manner similar to Example 86, Step 3 to provide (9H-fluoren-9-yl)methyl tert-butyl(6-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-6-oxohexane-1,5-diyl) (R)-dicarbamate (144 mg, 133 μmol, 38.2%) as a yellow solid. [M+H]=1083.7.

Step 2: (9H-fluoren-9-yl)methyl tert-butyl(6-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-6-oxohexane-1,5-diyl) (R)-dicarbamate was treated with DBU in a manner similar to Example 78, Step 9 to provide tert-butyl(R)-(6-amino-1-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-1-oxohexan-2-yl) carbamate (140 mg, 0.13 mmol, 100%) as reaction solution was used for next step without purification.

Step 3: tert-butyl(R)-(6-amino-1-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-1-oxohexan-2-yl) carbamate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide (R)-2,2′,2″-(10-(2-((5-((tert-butoxycarbonyl)amino)-6-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-6-oxohexyl)amino)-2-oxoethyl)-1,4,7,10-tetraazacyclododecane-1,4,7-triyl)triacetic acid (95 mg, 76 μmol, 59%) as a yellow solid. [M+H]=1248.1.

(R)-2,2′,2″-(10-(2-((5-((tert-butoxycarbonyl)amino)-6-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-6-oxohexyl)amino)-2-oxoethyl)-1,4,7,10-tetraazacyclododecane-1,4,7-triyl)triacetic acid was treated with TFA in a manner similar to Example 79, Step 8 to provide (R)-2,2′,2″-(10-(2-((5-amino-6-((4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-6-oxohexyl)amino)-2-oxoethyl)-1,4,7,10-tetraazacyclododecane-1,4,7-triyl)triacetic acid--2,2,2-trifluoroacetic acid (1/1) (36.1 mg, 28.6 μmol, 40%) as a white solid. MS: Calc'd for C₅₇H₇₃F5N₁₂O₁₅:1260.52, found [M+H−TFA]: 1147.5.

### Example 88:2,2′,2″-(10-(1-((5-(1-(3,5-difluorophenyl)-3-(3,3-dimethylmorpholine-4carbonyl)-7-methoxy-1,4-dihydrochromeno|4,3-c]pyrazol-8-yl) pyridin-3-yl)amino)-3,6,9,12,15-pentamethyl-1,4,7,10,13,16,20-heptaoxo-3,6,9,12,15,19-hexaazahenicosan-21-yl)-1,4,7,10-tetraazacyclododecane-1,4,7-triyl)triacetic acid--formic acid (1/1) (Compound 59)

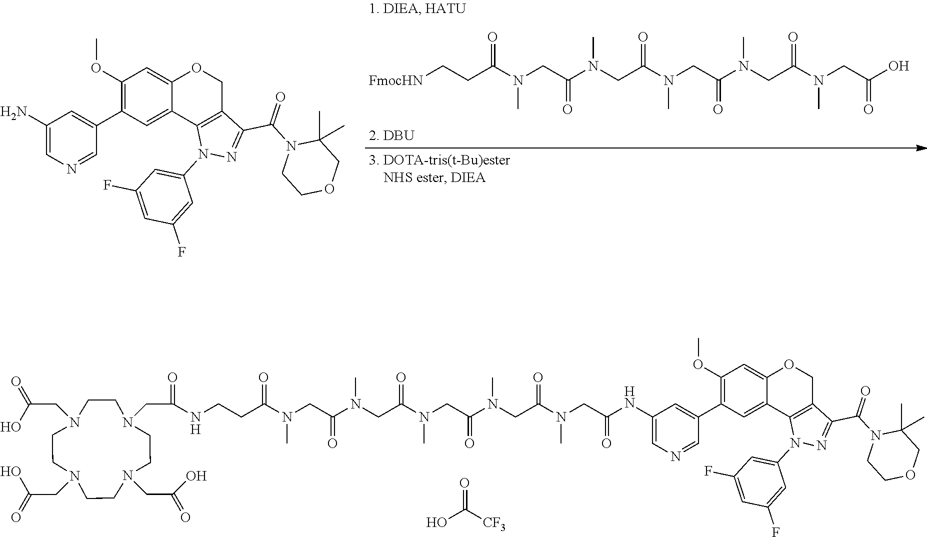

Step 1: (8-(5-aminopyridin-3-yl)-1-(3,5-difluorophenyl)-7-methoxy-1,4-dihydrochromeno[4,3-c]pyrazol-3-yl) (3,3-dimethylmorpholino) methanone was treated with 1-(9H-fluoren-9-yl)-8, 11, 14, 17,20-pentamethyl-3,7, 10, 13, 16, 19-hexaoxo-2-oxa-4,8, 11, 14, 17,20-hexaazadocosan-22-oic acid, DIEA, and HATU in a manner similar to Example 78, Step 6 to provide (9H-fluoren-9-yl)methyl (1-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3,6,9, 12, 15-pentamethyl-1,4,7, 10, 13, 16-hexaoxo-3,6,9, 12, 15-pentaazaoctadecan-18-yl) carbamate (145 mg, 121 μmol, 66.4%) as a yellow oil. [M+H]=1196.7.

Step 2: (9H-fluoren-9-yl)methyl (1-((5-(1-(3,5-difluorophenyl)-3-(3,3-yl)amino)-3,6,9,12, 15-pentamethyl-1,4,7,10, 13, 16-hexaoxo-3,6,9,12, 15-pentaazaoctadecan-18-yl) carbamate was treated with DBU in a manner similar to Example 78, Step 9 to provide 3-amino-N-(14-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3,6,9,12-tetramethyl-2,5,8,11,14-pentaoxo-3,6,9,12-tetraazatetradecyl)-N-methylpropanamide (75 mg, 77 μmol, 92%). [M+H]=974.6.

Step 3: 3-amino-N-(14-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3,6,9,12-tetramethyl-2,5,8, 11, 14-pentaoxo-3,6,9,12-tetraazatetradecyl)-N-methylpropanamide (75 mg, 1 Eq, 77 μmol)_{w} as treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(1-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-3,6,9, 12,15-pentamethyl-1,4,7,10, 13, 16,20-heptaoxo-3,6,9, 12, 15, 19-hexaazahenicosan-21-yl)-1,4, 7, 10-tetraazacyclododecane-1,4,7-triyl)triacetic acid--formic acid (1/1) (61.3 mg, 43.6 μmol, 57%) as a yellow solid. MS: Calc'd for C₆₄H₈₅F2N₁₅O₁₉:1405.61, found [M+H−FA]: 1360.6.

### Example 89:2,2′,2″-(10-(2-(4-(N-(2-(2-(2-carboxyethoxy)ethoxy)ethyl)-4-((5-(1-(3,5difluorophenyl)-3-(3,3-dimethylmorpholine-4-carbonyl)-7-methoxy-1,4-dihydrochromeno|4,3-c|pyrazol-8-yl) pyridin-3-yl)amino)-4-oxobutanamido) piperidin-1-yl)-2-oxoethyl)-1,4,7,10-tetraazacyclododecane-1,4,7-triyl)triacetic acid--formic acid (1/1) (Compound 60)

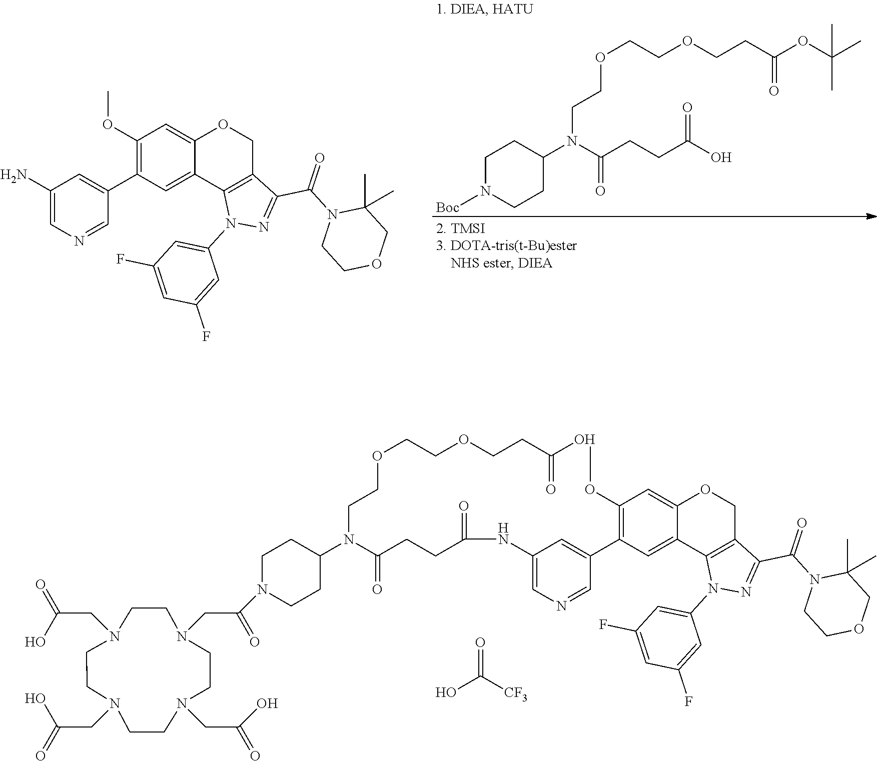 [SMILES: COc1cc2c(cc1-c1cncc(N)c1)-c1c(c(C(=O)N3CCOCC3(C)C)nn1-c1cc(F)cc(F)c1)CO2 ; CC(C)(C)OC(=O)CCOCCOCCN(C(=O)CCC(=O)O)C1CCN(C(=O)OC(C)(C)C)CC1 ; CC1(C)COCCN1C(=O)c1nn(-c2cc(F)cc(F)c2)c2c1COc1cc3c(cc1-2)-c1cncc(c1)NC(=O)CCC(=O)N(C1CCN(C(=O)CN2CCN(CC(=O)O)CCN(CC(=O)O)CCN(CC(=O)O)CC2)CC1)CCOCCOCCC(=O)OO3 ; O=C(O)C(F)(F)F]

Step 1: Into a 8-mL vial, was placed a mixture of 13-(1-(tert-butoxycarbonyl) piperidin-4-yl)-2,2-dimethyl-4,14-dioxo-3,7,10-trioxa-13-azaheptadecan-17-oic acid (110 mg, 1 Eq, 213 μmol), (8-(5-aminopyridin-3-yl)-1-(3,5-difluorophenyl)-7-methoxy-1,4-dihydrochromeno[4,3-c]pyrazol-3-yl) (3,3-dimethylmorpholino) methanone (175 mg, 1.50 Eq, 320 μmol), NMI (26.2 mg, 1.50 Eq, 319 μmol), TCFH (299 mg, 5.00 Eq, 1.07 mmol) and DMF (2 mL). The reaction mixture was stirred at 25° C. for an additional 2 hours. The mixture was directly purified by MPLC to provide tert-butyl 4-(N-(2-(2-(3-(tert-butoxy)-3-oxopropoxy)ethoxy)ethyl)-4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido) piperidine-1-carboxylate (95 mg, 91 μmol, 43%) as a yellow oil. [M+H]=1046.7.

Step 2: Into an 8-mL vial, was placed a mixture of tert-butyl 4-(N-(2-(2-(3-(tert-butoxy)-3-oxopropoxy)ethoxy)ethyl)-4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido) piperidine-1-carboxylate (90 mg, 1 Eq, 86 μmol) and MeCN (2 mL), to which was added TMS-I (86 mg, 59 μL, 5.0 Eq, 0.43 mmol). The reaction mixture was stirred at 25° C. for 1 hour. The mixture was directly purified by MPLC to provide 3-(2-(2- (4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxo-N-(piperidin-4-yl) butanamido) ethoxy)ethoxy) propanoic acid (40 mg, 45 μmol, 52%). [M+H]=890.5.

Step 3. 3-(2-(2-(4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxo-N-(piperidin-4-yl) butanamido) ethoxy)ethoxy) propanoic acid was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(2-(4-(N-(2-(2-(2-carboxyethoxy)ethoxy)ethyl)-4-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutanamido) piperidin-1-yl)-2-oxoethyl)-1,4,7,10-tetraazacyclododecane-1,4,7-triyl)triacetic acid--formic acid (1/1) (28.7 mg, 21.7 μmol, 48%) as a yellow solid. Calc'd for C₆₂H₈₁F2N₁₁O₁₉:1321. 57 found [M+H−FA]: 1276.6.

### Example 90: (R)-2,2′,2″-(10-(2-((1-carboxy-4-((2-((5-(1-(3,5-difluorophenyl)-3-(3,3dimethylmorpholine-4-carbonyl)-7-methoxy-1,4-dihydrochromeno|4,3-clpyrazol-8-yl) pyridin-3-yl)amino)-2-oxoethyl)amino)-4-oxobutyl)amino)-2-oxoethyl)-1,4,7,10-tetraazacyclododecane-1,4,7-triyl)triacetic acid (Compound 61)

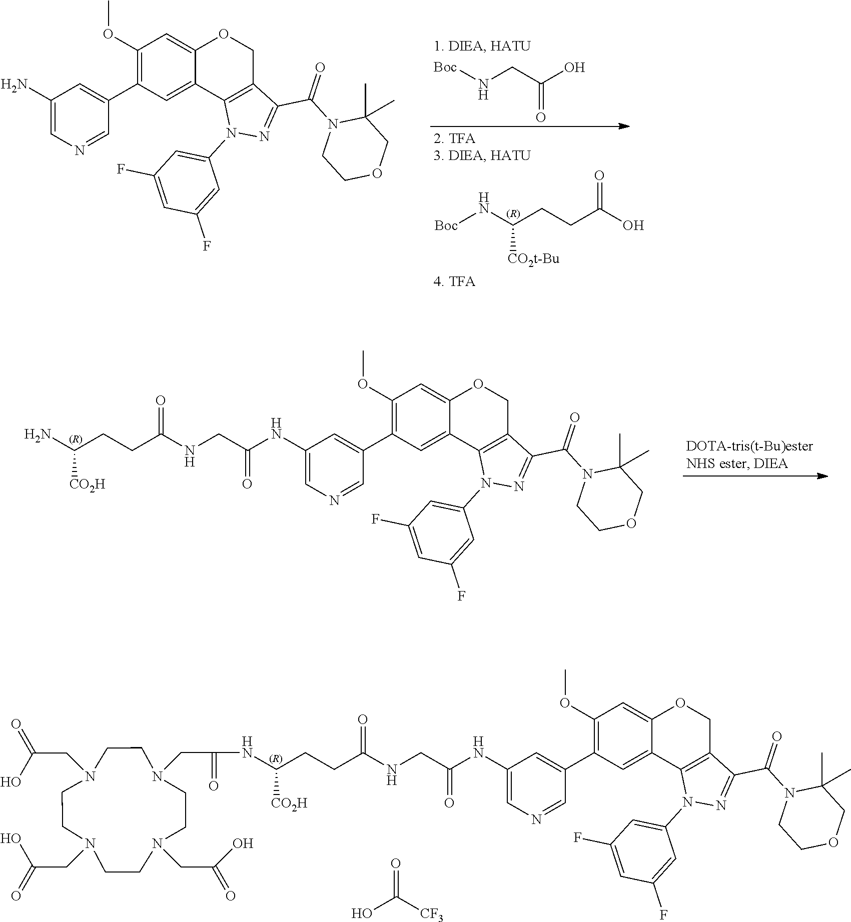

Step 1: (8-(5-aminopyridin-3-yl)-1-(3,5-difluorophenyl)-7-methoxy-1,4-dihydrochromeno[4,3-c]pyrazol-3-yl) (3,3-dimethylmorpholino) methanone (350 mg, 1 Eq, 639 μmol)_{w} as treated with (tert-butoxycarbonyl)glycine, DIEA, and HATU in a manner similar to Example 78, Step 6 to provide tert-butyl(2-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl) carbamate (300 mg, 426 μmol, 66.6%) as a yellow oil. [M+H]=705.5.

Step 2: tert-butyl(2-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl) carbamate was treated with TFA in a manner similar to Example 79, Step 8 to provide 2-amino-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)acetamide (400 mg, 0.40 mmol, 80%) as a yellow oil. [M+H]=605.5.

Step 3: 2-Amino-N-(5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)acetamide was treated with (R)-5-(tert-butoxy)-4-((tert-butoxycarbonyl)amino)-5-oxopentanoic acid, HATU and DIEA to provide tert-butyl N₂-(tert-butoxycarbonyl)-N₅-(2-((5-(1-(3,5-difluorophenyl)-3-(3,3-yl)amino)-2-oxoethyl)-D-glutaminate (115 mg, 129 μmol, 39.1%) as a yellow oil. [M+H]=890.4.

Step 4: tert-butyl N₂-(tert-butoxycarbonyl)-N₅-(2-((5-(1-(3,5-difluorophenyl)-3-(3,3-yl)amino)-2-oxoethyl)-D-glutaminate was treated with TFA in a manner similar to Example 79, Step 8 to provide N₅-(2-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-D-glutamine (110 mg, 90 μmol, 76%) as a yellow oil. [M+H]=734.3.

Step 5. N₅-(2-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)-D-glutamine was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide (R)-2,2′,2″-(10-(2-((1-carboxy-4-((2-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino)-2-oxoethyl)amino)-4-oxobutyl)amino)-2-oxoethyl)-1,4,7,10-tetraazacyclododecane-1,4,7-triyl)triacetic acid (39.1 mg, 34.9 μmol, 30.9%) as a white solid. Calc'd for C₅₂H₆₃F2N₁₁O₁₅:1119.44, found [M+H]: 1120.5.

### Example 91:2,2′,2″-(10-(2-((2-(3-((3-((5-(1-(3,5-difluorophenyl)-3-(3,3dimethylmorpholine-4-carbonyl)-7-methoxy-1,4-dihydrochromeno|4,3-c]pyrazol-8-yl) pyridin-3-yl)amino) propyl)amino)-3-oxopropoxy)ethyl)amino)-2-oxoethyl)-1,4,7,10-tetraazacyclododecane-1,4,7-triyl)triacetic acid--2,2,2-trifluoroacetic acid (1/1) (Compound 62)

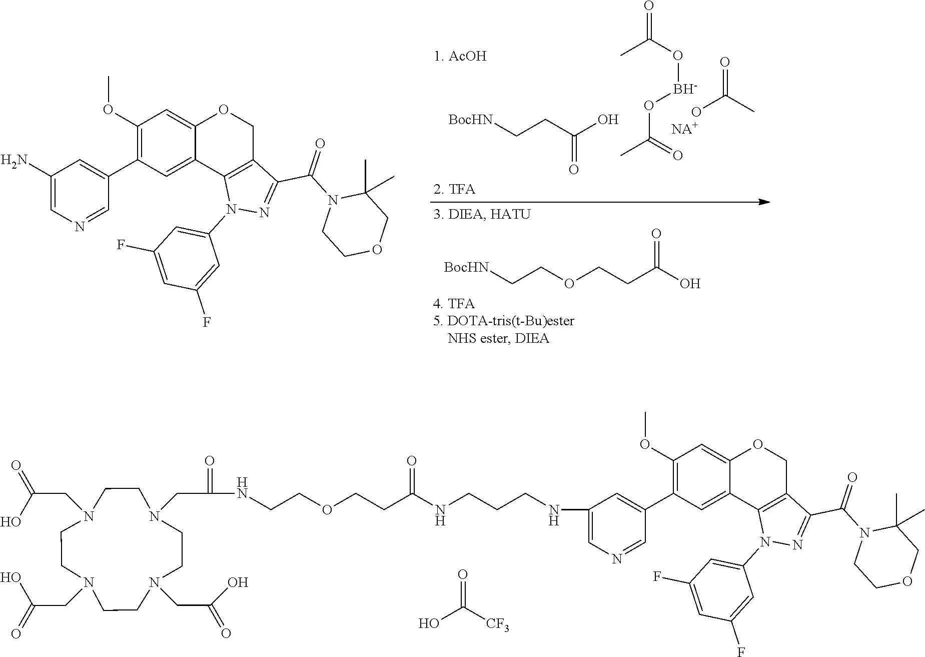

Step 1: Into a 40-mL vial, was placed a mixture of (8-(5-aminopyridin-3-yl)-1-(3,5-difluorophenyl)-7-methoxy-1,4-dihydrochromeno[4,3-c]pyrazol-3-yl) (3,3-dimethylmorpholino) methanone (300 mg, 1 Eq, 548 μmol), tert-butyl(3-oxopropyl) carbamate (142 mg, 1.50 Eq, 820 μmol), sodium triacetoxyhydroborate (230 mg, 1.98 Eq, 1.09 mmol), AcOH (33 mg, 31 μL, 1.0 Eq, 0.55 mmol) and DCE (3.0 mL). The reaction mixture was stirred at 25° C. for an additional 2 hours. The mixture was purified by MPLC to provide tert-butyl(3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino) propyl) carbamate (230 mg, 0.26 mmol, 48%) as a yellow oil. [M+H]=705.3.

Step 2: tert-butyl(3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino) propyl) carbamate was treated with TFA in a manner similar to Example 79, Step 8 to provide (8-(5-((3-aminopropyl)amino)pyridin-3-yl)-1-(3,5-difluorophenyl)-7-methoxy-1,4-dihydrochromeno[4,3-c]pyrazol-3-yl) (3,3-dimethylmorpholino) methanone (200 mg, 0.13 mmol, 41%) as a yellow oil. [M+H]=605.2.

Step 3: (8-(5-((3-aminopropyl)amino)pyridin-3-yl)-1-(3,5-difluorophenyl)-7-methoxy-1,4-dihydrochromeno[4,3-c]pyrazol-3-yl) (3,3-dimethylmorpholino) methanone was treated with 3-(2-((tert-butoxycarbonyl)amino) ethoxy) propanoic acid, HATU, and DIEA to provide tert-butyl (2-(3-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino) propyl)amino)-3-oxopropoxy)ethyl) carbamate (150 mg, 183 μmol, 56.7%) as a yellow oil. [M+H]=820.3.

Step 4: 3-(2-((tert-butoxycarbonyl)amino) ethoxy) propanoic acid, HATU, and DIEA to provide tert-butyl(2-(3-((3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino) propyl)amino)-3-oxopropoxy)ethyl) carbamate was treated with TFA in a manner similar to Example 79, step 8 to provide 3-(2-aminoethoxy)-N-(3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino) propyl) propanamide (100 mg, 0.11 mmol, 63%) as a yellow oil. [M+H]=960.7. Step 5. 3-(2-aminoethoxy)-N-(3-((5-(1-(3,5-difluorophenyl)-3-(3,3-dimethylmorpholine-4-carbonyl)-7-methoxy-1,4-dihydrochromeno[4,3-c]pyrazol-8-yl) pyridin-3-yl)amino) propyl) propanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(2-((2-(3-((3-((5-(1-(3,5-difluorophenyl)-3-(3,3-yl)amino) propyl)amino)-3-oxopropoxy)ethyl)amino)-2-oxoethyl)-1,4,7,10-tetraazacyclododecane-1,4,7-triyl)triacetic acid--2,2,2-trifluoroacetic acid (1/1) as an off-white solid. Calc'd for C₅₅H₇₀F5N₁₁O₁₅:1219.50, found [M+H−TFA]⁺: 1106.5.

### Example 92:2,2′,2″-(10-((4R,7R,10R,13R,16R,19R)-19-(((R)-1-amino-1-oxopropan-2yl) carbamoyl)-7,13-bis(2-carboxyethyl)-28-((3-(1-(3,5-dichlorophenyl)-3-(3,3 dimethylmorpholine-4-carbonyl)-7-methoxy-1,4-dihydrochromeno[4,3-c]pyrazol-8-yl)phenyl)amino)-4,10,16-trimethyl-2,5,8,11,14,17,25,28-octaoxo-3,6,9,12,15,18,24-heptaazaoctacosyl)-1,4,7,10-tetraazacyclododecane-1,4,7-triyl)triacetic acid (Compound 63)

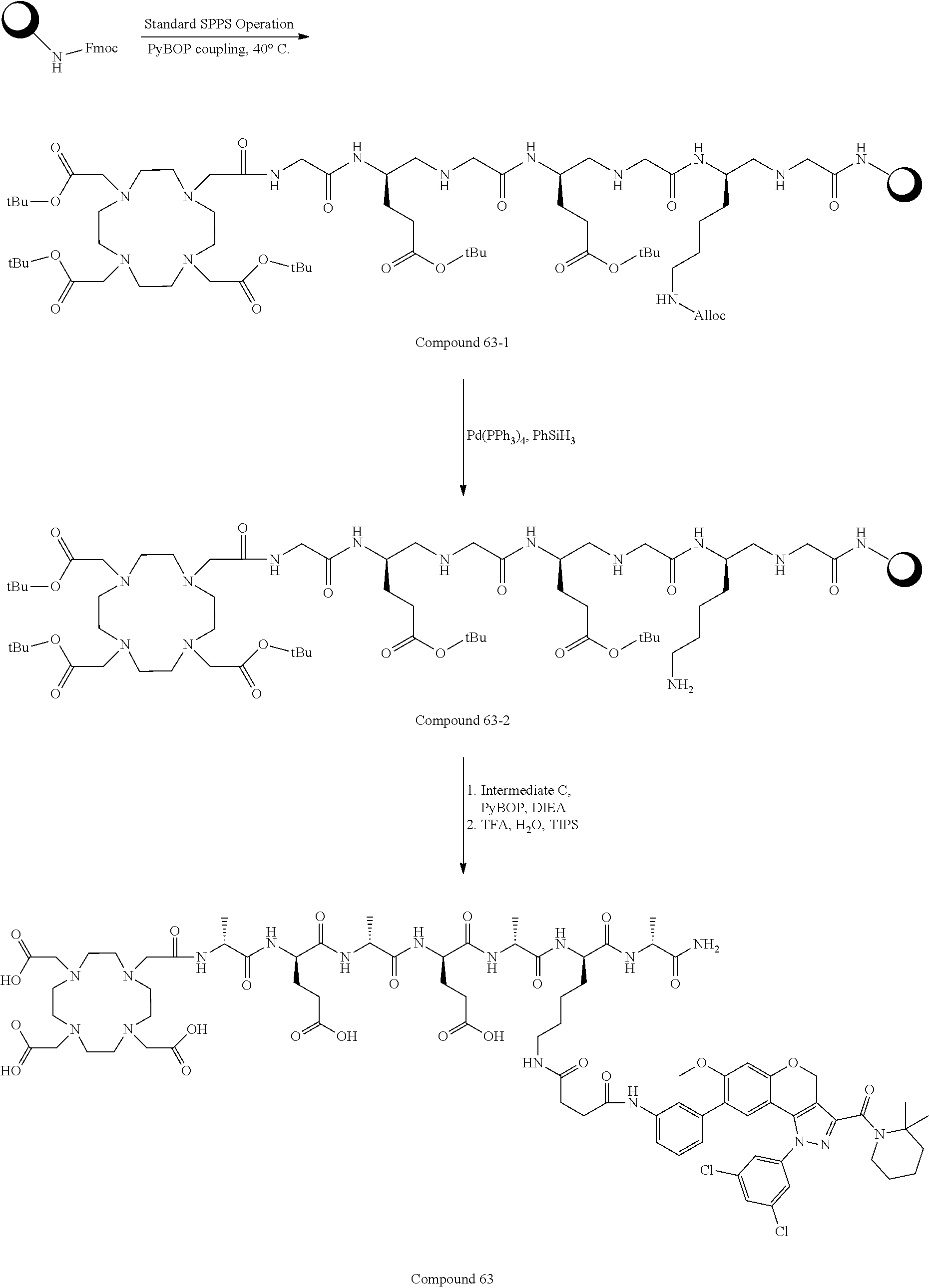

VI

Step 1. Synthesis of Compound 63-1: The automated SPPS was executed with a Syro II peptide synthesizer (Biotage). Resin pre-swelling: The Rink amide resin (100 mg/tube, 100-200 mesh, 0.62 mmol/g, total 5 tubes)_{w} as swelled in NMP (1 mL/tube) for 15 min at room temperature under nitrogen atmosphere. The resin was then washed 4 times with NMP (1 mL/tube). Parameter: incubation time: 15 min, vortex time: 15 s, break time: 1 min, temperature: room temperature, empty time: 1 min. SPPS coupling cycle: A) Fmoc de-protection: the resin (100 mg/tube, total 5 tubes)_{w} as treated with 20% of piperidine in NMP (1 mL) for 20 min at room temperature under a nitrogen atmosphere. The resin was then washed 4 times with NMP (1 mL/tube). Parameter: reaction time: 20 min, vortex time: 15 s, break time: 1 min, temperature: room temperature, empty time: 1 min. B) PyBOP coupling: the resin (100 mg/tube, total 5 tubes) was treated with the mixture of amino acid or DOTA-(OrBu) 3 (4 eq.), PyBOP (4 eq.) and DIPEA (8 eq.) in NMP (1 mL) for 45 min at 40° C. under a nitrogen atmosphere. Then, the resin was washed 4 times with NMP (1 mL/tube). Parameter: reaction time: 45 min, vortex time: 15 s, break time: 3 min, temperature: 30° C., empty time: 1 min.

Step 2. Synthesis of Compound 63-2: A) Compound 63-1 on resin (100 mg)_{w} as swelled with DCE (2 mL) for 15 min at room temperature. The solvent was then removed. To the resin (100 mg) in 10 mL reactor was added the mixture of Pd(PPh3)+ (0.5 eq.) and PhSiH3 (20 eq.) in DCE (2 mL) at room temperature under nitrogen atmosphere. The mixture was bubbled for 2 h and filtered, washed with DCE (2 mL×3), NMP (2 mL×3).

Step 3: To the resin (100 mg) in 10 mL reactor were added Intermediate C (2 eq.), PyBOP (4 eq.) and DIPEA (8 eq.) in NMP (1 mL). After the reaction mixture was incubated for 2 h at room temperature, the solvent was removed and resin was washed with NMP (5 mL×6).

Step 4: The resin was treated with the cleavage solution (v v v TFA/H₂O/TIS=37:1:1) for 2 h at room temperature. After filtration, the filtrate was concentrated under reduced pressure. The residue was precipitated with cold ether. After centrifugation, the solid was collected and purified by prep-HPLC. The desired fractions were combined and lyophilized to afford Compound 63 as a white solid (4.9 mg, 5.1%). Calc'd for C₇&H₁₀₅C₁₂N₁₇O₂₄:1733.7, found [M+2H]²⁺: 868.7.

### Example 93: (R)-2,2′,2″-(10-(2-((1-((6-((3-(1-(3,5-dichlorophenyl)-3-(3,3dimethylmorpholine-4-carbonyl)-7-methoxy-1,4-dihydrochromeno|4,3-c]pyrazol-8-yl)phenyl)amino)-6-oxohexyl)amino)-1-oxo-3-sulfopropan-2-yl)amino)-2-oxoethyl)-1,4,7,10-tetraazacyclododecane-1,4,7-triyl)triacetic acid (Compound 64)

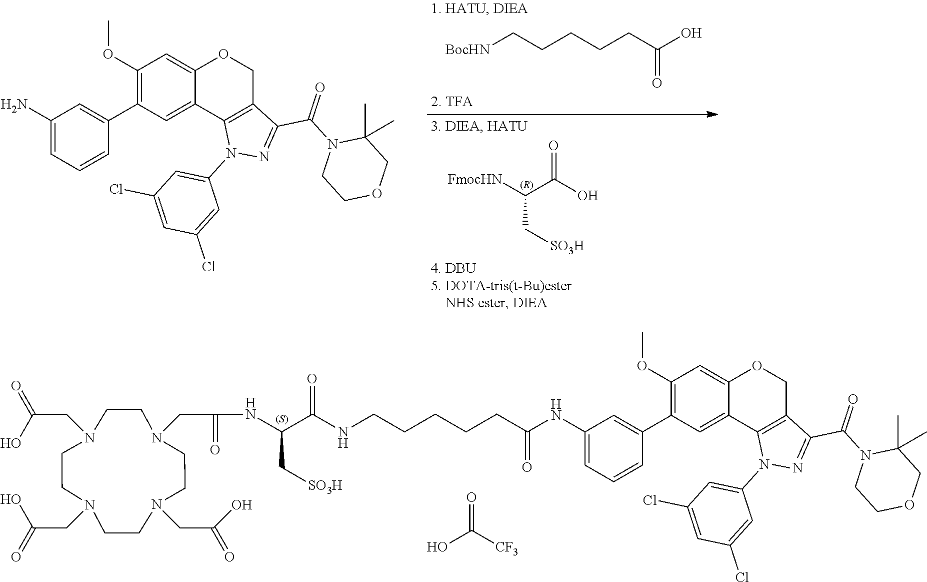

Step 1: (8-(3-aminophenyl)-1-(3,5-dichlorophenyl)-7-methoxy-1,4-dihydrochromeno[4,3-c]pyrazol-3-yl) (3,3-dimethylmorpholino) methanone was treated with 6-((tert-butoxycarbonyl)amino) hexanoic acid, HATU and DIEA in a manner similar to Example 78, Step 6 to provide tert-butyl(6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-6-oxohexyl) carbamate (220 mg, 278 μmol, 80.4%) as a yellow solid. [M+H]=792.3, 794.3.

Step 2: tert-Butyl (6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-6-oxohexyl) carbamate was treated with TFA in a manner similar to Example 79, Step 8 to provide 6-amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) hexanamide as a yellow crude oil. [M+H]=692.2.

Step 3: 6-Amino-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl) hexanamide was treated with (((9H-fluoren-9-yl) methoxy) carbonyl) (sulfo)-D-alanine, HATU and DIEA to provide (R)-2-((((9H-fluoren-9-yl) methoxy) carbonyl)amino)-3-((6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-6-oxohexyl)amino)-3-oxopropane-1-sulfonic acid (170 mg, 159 μmol, 50.2%) as a yellow oil.

Step 4: (R)-2-((((9H-fluoren-9-yl) methoxy) carbonyl)amino)-3-((6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-6-oxohexyl)amino)-3-oxopropane-1-sulfonic acid was treated with DBU in a manner similar to Example 78, step 9 to provide crude (R)-2-amino-3-((6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-6-oxohexyl)amino)-3-oxopropane-1-sulfonic acid. [M+H]=843.2, 845.2.

Step 5. (R)-2-amino-3-((6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-6-oxohexyl)amino)-3-oxopropane-1-sulfonic acid was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide (R)-2,2′,2″-(10-(2-((1-((6-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-6-oxohexyl)amino)-1-oxo-3-sulfopropan-2-yl)amino)-2-oxoethyl)-1,4,7,10-tetraazacyclododecane-1,4,7-triyl)triacetic acid (78 mg, 63 μmol, 31%) as a white solid. Calc'd for C₅₅H₇₀C₁₂N₁₀O₁₆S: 1228.41, found [M+H]⁺: 1229.3, 1231.3.

### Example 94:2,2′,2″-(10-(2-(((R)-1-((((1r,4R)-4-((3-(1-(3,5-dichlorophenyl)-3-(3,3dimethylmorpholine-4-carbonyl)-7-methoxy-1,4-dihydrochromeno|4,3-clpyrazol-8-yl)phenyl) carbamoyl)cyclohexyl)methyl)amino)-1-oxo-3-sulfopropan-2-yl)amino)-2-oxoethyl)-1,4,7,10-tetraazacyclododecane-1,4,7-triyl)triacetic acid (Compound 65)

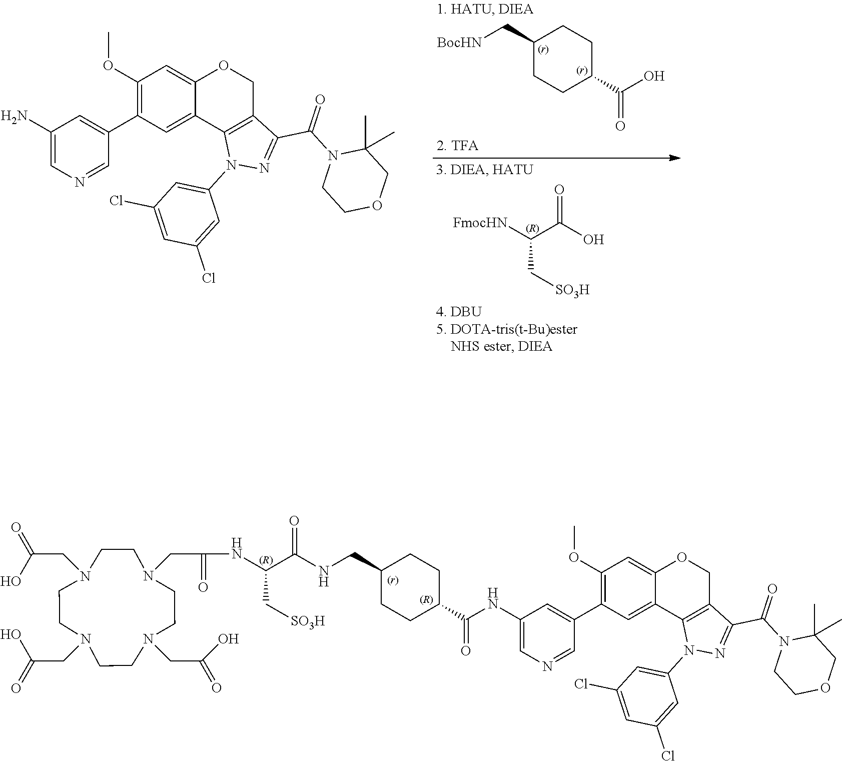

Step 1: (8-(3-aminophenyl)-1-(3,5-dichlorophenyl)-7-methoxy-1,4-dihydrochromeno[4,3-c]pyrazol-3-yl) (3,3-dimethylmorpholino) methanone is treated with (1r,4r)-4-(((tert-butoxycarbonyl)amino)methyl)cyclohexane-1-carboxylic acid (98 mg, 1.1 Eq, 0.38 mmol), HATU and DIEA to provide tert-butyl(((1r,4r)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)cyclohexyl)methyl) carbamate (230 mg, 281 μmol, 81.4%) as a white solid.

[M+H]=818.3, 820.3.

Step 2: tert-butyl(((1r,4r)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)cyclohexyl)methyl) carbamate was treated with TFA in a manner similar to Example 79, Step 8 to provide (1r,4r)-4-(aminomethyl)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)cyclohexane-1-carboxamide (185 mg, 257 μmol, 91.6%) as a light yellow solid, which was used directly in the next step without any purification. [M+H]=718.3, 720.3.

Step 3: (1r,4r)-4-(aminomethyl)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)cyclohexane-1-carboxamide was treated with (((9H-fluoren-9-yl) methoxy) carbonyl) (sulfo)-D-alanine, HATU and DIEA in a manner similar to Example 78, Step 6 to provide (R)-2-((((9H-fluoren-9-yl) methoxy) carbonyl)amino)-3-((((1r,4R)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)cyclohexyl)methyl)amino)-3-oxopropane-1-sulfonic acid (120 mg, 110 μmol, 42.7%) as a white solid. [M+H]=1091.1, 1093.1.

Step 4: (R)-2-((((9H-fluoren-9-yl) methoxy) carbonyl)amino)-3-((((1r,4R)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)cyclohexyl)methyl)amino)-3-oxopropane-1-sulfonic acid was treated with DBU in a manner similar to Example 78, Step 9 to provide (R)-2-amino-3-((((1r,4R)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)cyclohexyl)methyl)amino)-3-oxopropane-1-sulfonic acid (70 mg, 80 μmol, 73%) as a crude yellow oil, which was used directly in the next step without any further purification. [M+H]=869.2, 871.2.

Step 5: (R)-2-amino-3-((((1r,4R)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)cyclohexyl)methyl)amino)-3-oxopropane-1-sulfonic acid was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide 2,2′,2″-(10-(2-(((R)-1-((((1r,4R)-4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl) carbamoyl)cyclohexyl)methyl)amino)-1-oxo-3-sulfopropan-2-yl)amino)-2-oxoethyl)-1,4,7,10-tetraazacyclododecane-1,4,7-triyl)triacetic acid (42.3 mg, 33.7 μmol, 49%) as a white solid. Calc'd for C₅₇H₇₂C₁₃N₁₀O₁₆S: 1254.42, found [M+H]⁺: 1255.6, 1257.6.

### Example 95: (R)-2,2′,2″-(10-(2-((1-((1-(2-((3-(1-(3,5-dichlorophenyl)-3-(3,3dimethylmorpholine-4-carbonyl)-7-methoxy-1,4-dihydrochromeno|4,3-c]pyrazol-8-yl)phenyl)amino)-2-oxoethyl) piperidin-4-yl)amino)-1-oxo-3-sulfopropan-2-yl)amino)-2-oxoethyl)-1,4,7,10-tetraazacyclododecane-1,4,7-triyl)triacetic acid--2,2,2-trifluoroacetic acid (1/1) (Compound 66)

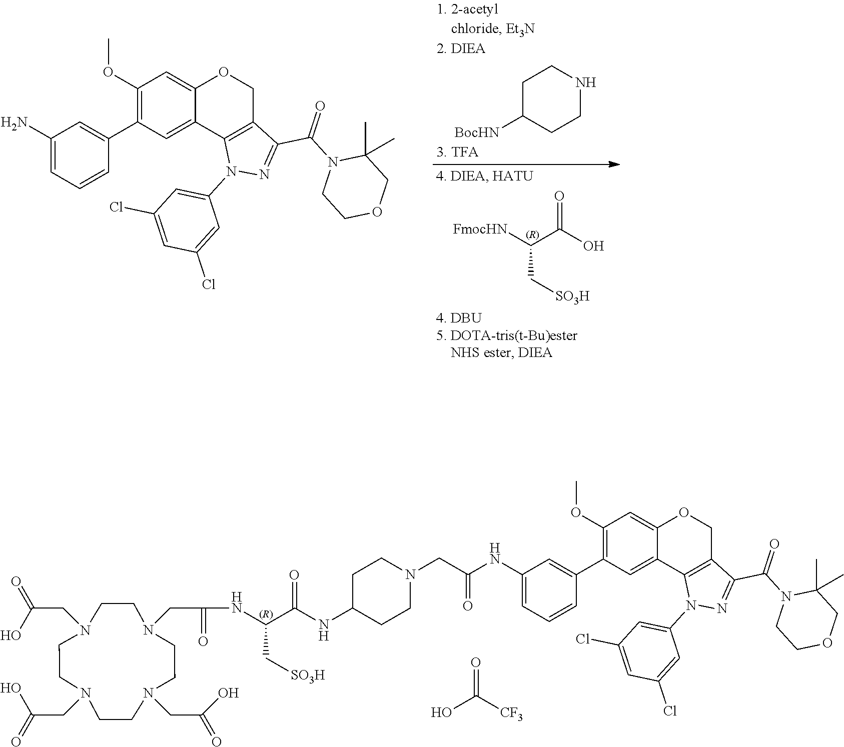

Step 1: Into a 8-mL vial, was placed a mixture of (8-(3-aminophenyl)-1-(3,5-dichlorophenyl)-7-methoxy-1,4-dihydrochromeno[4,3-c]pyrazol-3-yl) (3,3-dimethylmorpholino) methanone (200 mg, 1 Eq, 345 μmol), Et₃N (105 mg, 145 μL, 3.01 Eq, 1.04 mmol) and DCM (2 mL), to which was added 2-chloroacetyl chloride (60 mg, 1.5 Eq, 0.53 mmol). The reaction mixture was stirred at 0° C. for 1 hour then concentrated under reduced pressure. The crude product was purified by MPLC to provide 2-chloro-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (160 mg, 244 μmol, 70.7%) as a white solid. [M+H]=655.4, 657.4.

Step 2: Into a 8-mL vial, was placed a mixture of 2-chloro-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (160 mg, 1 Eq, 244 μmol), DIEA (95 mg, 3.0 Eq, 0.74 mmol), tert-butyl piperidin-4-ylcarbamate (59 mg, 1.2 Eq, 0.29 mmol) and DMF (2 mL). The reaction mixture was stirred at 60° C. for 2 hours. The mixture was directly purified by MPLC to provide tert-butyl(1-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) piperidin-4-yl) carbamate (160 mg, 195 μmol, 80.0%) as a yellow solid. [M+H]=819.2, 821.2.

Step 3: tert-butyl(1-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) piperidin-4-yl) carbamate was treated with TFA in a manner similar to Example 78, Step 8 to provide 2-(4-aminopiperidin-1-yl)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide (135 mg, 188 μmol, 96.1%) as a yellow oil. [M+H]=719.5, 721.5.

Step 4: 2-(4-Aminopiperidin-1-yl)-N-(3-(1-(3,5-dichlorophenyl)-3-(3,3-dimethylmorpholine-4-carbonyl)-7-methoxy-1,4-dihydrochromeno[4,3-c]pyrazol-8-yl)phenyl)acetamide was treated with (((9H-fluoren-9-yl) methoxy) carbonyl) (sulfo)-D-alanine, HATU and DIEA in a manner similar to Example 78, Step 6 to provide (R)-2-((((9H-fluoren-9-yl) methoxy) carbonyl)amino)-3-((1-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) piperidin-4-yl)amino)-3-oxopropane-1-sulfonic acid (137 mg, 125 μmol, 66.8%) as a yellow oil. [M+H]=1092.3, 1094.3.

Step 5: (R)-2-((((9H-fluoren-9-yl) methoxy) carbonyl)amino)-3-((1-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) piperidin-4-yl)amino)-3-oxopropane-1-sulfonic acid was treated with DBU in a manner similar to Example 78, Step 9 to provide (R)-2-amino-3-((1-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) piperidin-4-yl)amino)-3-oxopropane-1-sulfonic acid (110 mg, 0.11 mmol, 92%) as a white solid. [M+H]=870.6, 872.6.

Step 6: (R)-2-amino-3-((1-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) piperidin-4-yl)amino)-3-oxopropane-1-sulfonic acid was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide (R)-2,2′,2″-(10-(2-((1-((1-(2-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2-oxoethyl) piperidin-4-yl)amino)-1-oxo-3-sulfopropan-2-yl)amino)-2-oxoethyl)-1,4,7,10-tetraazacyclododecane-1,4,7-triyl)triacetic acid--2,2,2-trifluoroacetic acid (1/1) (97.9 mg, 71.4 μmol, 56.5%) as a white solid. Calc'd for C₅₈H₇₂C₁₂F3N₁₁O₁₈S: 1369.41, found [M+H−TFA]⁺: 1256.6, 1258.6.

### Example 96: (R)-2,2′,2″-(10-(20-((5-(1-(3,5-dichlorophenyl)-3-(3,3-dimethylmorpholine-4carbonyl)-7-methoxy-1,4-dihydrochromeno|4,3-c|pyrazol-8-yl) pyridin-3-yl)amino)-2,5-dioxo-4-(sulfomethyl)-9,12,15,18-tetraoxa-3,6-diazaicosyl)-1,4,7,10-tetraazacyclododecane-1,4,7-triyl)triacetic acid--2,2,2-trifluoroacetic acid (1/1) (Compound 67)

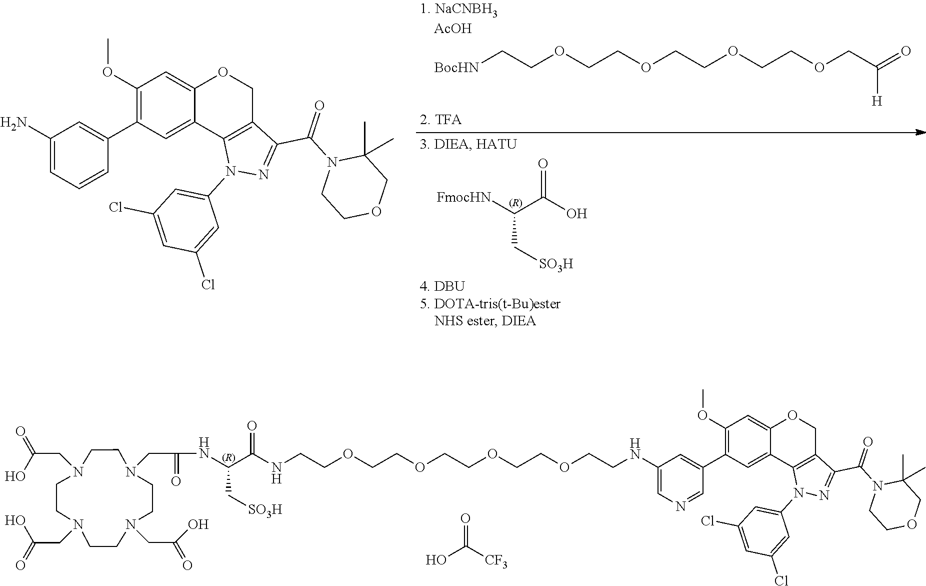

Step 1: Into an 8-mL vial, was placed a mixture of (8-(5-aminopyridin-3-yl)-1-(3,5-dichlorophenyl)-7-methoxy-1,4-dihydrochromeno[4,3-c]pyrazol-3-yl) (3,3-dimethylmorpholino) methanone (80 mg, 1 Eq, 0.14 mmol), sodium cyanoborohydride (17 mg, 16 μL, 2.0 Eq, 0.27 mmol), tert-butyl(14-oxo-3,6,9,12-tetraoxatetradecyl) carbamate (55 mg, 1.2 Eq, 0.16 mmol), AcOH (17 mg, 16 μL, 2.1 Eq, 0.28 mmol) and MeOH (1.0 mL). The reaction mixture was stirred at 25° C. for 2 hours then the reaction mixture was directly purified by MPLC to provide tert-butyl(14-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3,6,9,12-tetraoxatetradecyl) carbamate (80 mg, 89 μmol, 65%) as a yellow oil. [M+H]=899.7.

Step 2: tert-butyl(14-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3,6,9,12-tetraoxatetradecyl) carbamate was treated with TFA in a manner similar to Example 79, Step 8 to provide (8-(5-((14-amino-3,6,9,12-tetraoxatetradecyl)amino)pyridin-3-yl)-1-(3,5-dichlorophenyl)-7-methoxy-1,4-dihydrochromeno[4,3-c]pyrazol-3-yl) (3,3-dimethylmorpholino) methanone (70 mg, 88 μmol, 98%) as a white solid. [M+H]=799.3.

Step 3: (8-(5-((14-amino-3,6,9,12-tetraoxatetradecyl)amino)pyridin-3-yl)-1-(3,5-dichlorophenyl)-7-methoxy-1,4-dihydrochromeno[4,3-c]pyrazol-3-yl) (3,3-dimethylmorpholino) methanone was treated with (((9H-fluoren-9-yl) methoxy) carbonyl) (sulfo)-D-alanine, HATU and DIEA in a manner similar to Example 78, Step 6 to provide (R)-17-((((9H-fluoren-9-yl) methoxy) carbonyl)amino)-1-((5-(1-(3,5-dichlorophenyl)-3-(3,3-yl)amino)-16-oxo-3,6,9,12-tetraoxa-15-azaoctadecane-18-sulfonic acid (50 mg, 43 μmol, 49%) as a yellow oil. [M+H]=1032.1.

Step 4: (R)-17-((((9H-fluoren-9-yl) methoxy) carbonyl)amino)-1-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-16-oxo-3,6,9,12-tetraoxa-15-azaoctadecane-18-sulfonic acid was treated with DBU in a manner similar to Example 79, Step 9 to provide (R)-17-amino-1-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-16-oxo-3,6,9,12-tetraoxa-15-azaoctadecane-18-sulfonic acid (30 mg, 32 μmol, 74%) as a yellow oil. [M+H]=873.2.

Step 5: (R)-17-Amino-1-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-16-oxo-3,6,9,12-tetraoxa-15-azaoctadecane-18-sulfonic acid was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide (R)-2,2′,2″-(10-(20-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2,5-dioxo-4-(sulfomethyl)-9,12,15,18-tetraoxa-3,6-diazaicosyl)-1,4, 7, 10-tetraazacyclododecane-1,4,7-triyl)triacetic acid--2,2,2-trifluoroacetic acid (1/1) (30 mg, 21 μmol, 49%) as a yellow oil. Calc'd for C₆₀H₈₀C₁₂F3N₁₁O₂₁S: 1449.45, found [M+H−TFA]⁺: 1336.5.

### Example 97: (R)-2,2′,2″-(10-(20-((5-(1-(3,5-dichlorophenyl)-3-(3,3-dimethylmorpholine-4carbonyl)-7-methoxy-1,4-dihydrochromeno|4,3-c|pyrazol-8-yl) pyridin-3-yl)oxy)-2,5-dioxo-4-(sulfomethyl)-9,12,15,18-tetraoxa-3,6-diazaicosyl)-1,4,7,10-tetraazacyclododecane-1,4,7-triyl)triacetic acid (Compound 68)

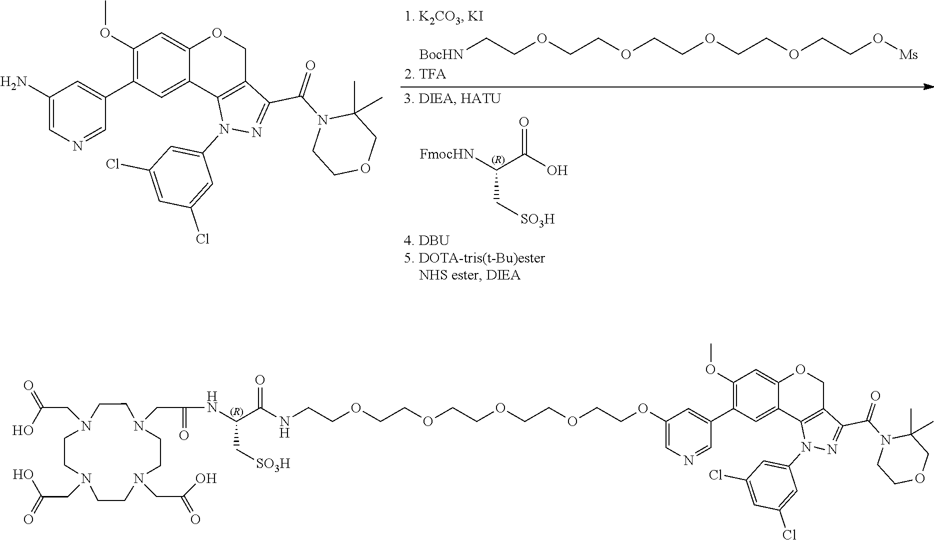

Step 1: Into a 8-mL vial, was placed a mixture of (1-(3,5-dichlorophenyl)-8-(5-hydroxypyridin-3-yl)-7-methoxy-1,4-dihydrochromeno[4,3-c]pyrazol-3-yl) (3,3-dimethylmorpholino) methanone (110 mg, 1 Eq, 189 μmol), 2,2-dimethyl-4-oxo-3,8,11,14,17-pentaoxa-5-azanonadecan-19-yl methanesulfonate (95 mg, 1.2 Eq, 0.23 mmol), K₂CO₃ (78 mg, 3.0 Eq, 0.56 mmol), KI (30 mg, 0.96 Eq, 0.18 mmol) and DMF (1.1 mL). The reaction mixture was stirred at 100° C. for 16 hours. The mixture was directly purified by MPLC to provide tert-butyl(14-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)oxy)-3,6,9,12-tetraoxatetradecyl) carbamate (85 mg, 94 μmol, 50%) as a white solid. [M+H]=900.2.

Step 2: tert-butyl(14-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)oxy)-3,6,9,12-tetraoxatetradecyl) carbamate was treated with TFA in a manner similar to Example 79, step 8 to provide (8-(5-((14-amino-3,6,9,12-tetraoxatetradecyl)oxy)pyridin-3-yl)-1-(3,5-dichlorophenyl)-7-methoxy-1,4-dihydrochromeno[4,3-c]pyrazol-3-yl) (3,3-dimethylmorpholino) methanone (85 mg, 85 μmol, 90%) as a yellow oil. [M+H]=800.1.

Step 3: (8-(5-((14-amino-3,6,9,12-tetraoxatetradecyl)oxy)pyridin-3-yl)-1-(3,5-dichlorophenyl)-7-methoxy-1,4-dihydrochromeno[4,3-c]pyrazol-3-yl) (3,3-dimethylmorpholino) methanone was treated with (((9H-fluoren-9-yl) methoxy) carbonyl) (sulfo)-D-alanine, HATU and DIEA to provide (R)-17-((((9H-fluoren-9-yl) methoxy) carbonyl)amino)-1-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)oxy)-16-oxo-3,6,9,12-tetraoxa-15-azaoctadecane-18-sulfonic acid. [M−H]=1173.6.

Step 4: (R)-17-((((9H-fluoren-9-yl) methoxy) carbonyl)amino)-1-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)oxy)-16-oxo-3,6,9,12-tetraoxa-15-azaoctadecane-18-sulfonic acid was treated with DBU in a manner similar to Example 78, Step 9 to provide (R)-17-amino-1-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)oxy)-16-oxo-3,6,9,12-tetraoxa-15-azaoctadecane-18-sulfonic acid (66 mg, 69 μmol) as a white solid. [M+H]=953.5.

Step 5. (R)-17-amino-1-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)oxy)-16-oxo-3,6,9,12-tetraoxa-15-azaoctadecane-18-sulfonic acid was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 14 of Example 1 to provide (R)-2,2′,2″-(10-(20-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)oxy)-2,5-dioxo-4-(sulfomethyl)-9,12,15,18-tetraoxa-3,6-diazaicosyl)-1,4,7,10-tetraazacyclododecane-1,4,7-triyl)triacetic acid as a white solid. Calc'd for C₅₈H₇₈C₁₂N₁₀O₂₀S: 1336.45, found [M+H−TFA]]: 1337.5.

### Example 98: (R)-2,2′,2″-(10-(15-((3-(1-(3,5-dichlorophenyl)-3-(3,3-dimethylmorpholine-4carbonyl)-7-methoxy-1,4-dihydrochromeno|4,3-c]pyrazol-8-yl)phenyl)amino)-2,5,15-trioxo-4-(sulfomethyl)-9,12-dioxa-3,6-diazapentadecyl)-1,4,7,10-tetraazacyclododecane-1,4,7-triyl)triacetic acid--2,2,2-trifluoroacetic acid (1/1) (Compound 69)

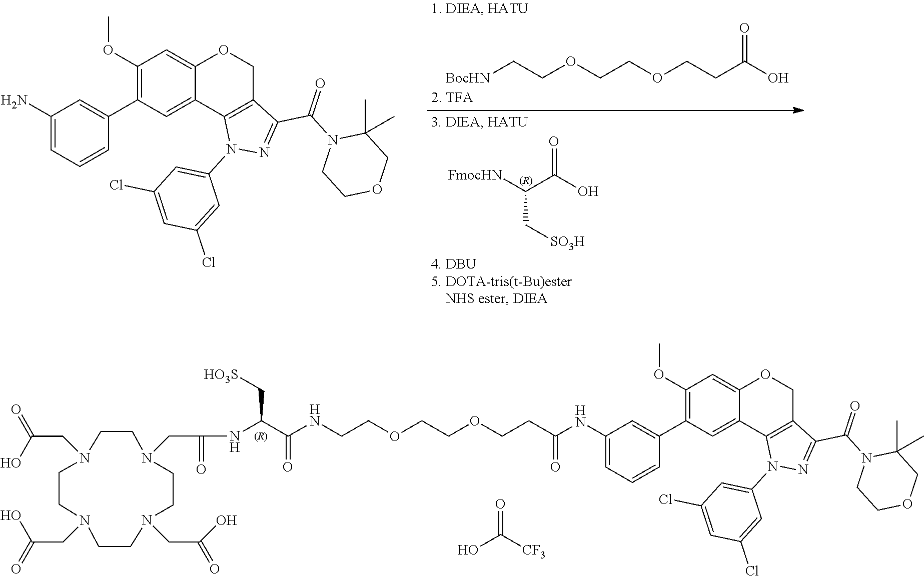

Step 1: (8-(3-aminophenyl)-1-(3,5-dichlorophenyl)-7-methoxy-1,4-dihydrochromeno[4,3-c]pyrazol-3-yl) (3,3-dimethylmorpholino) methanone was treated with 2,2-dimethyl-4-oxo-3,8, 11-trioxa-5-azatetradecan-14-oic acid, HATU and DIEA in a manner similar to Example 78, Step 6 to provide tert-butyl(2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamate (180 mg, 215 μmol, 82.9%) as a yellow solid. [M+H]=838.2.

Steps 2-5. (R)-2,2′,2″-(10-(15-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,5,15-trioxo-4-(sulfomethyl)-9,12-dioxa-3,6-diazapentadecyl)-1,4,7,10-tetraazacyclododecane-1,4,7-triyl)triacetic acid--2,2,2-trifluoroacetic acid (1/1)_{w} as synthesized from tert-Butyl (2-(2-(3-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-3-oxopropoxy)ethoxy)ethyl) carbamate in four steps in a manner similar to Example 94, steps 2-5 to provide the product (39.4 mg, 28.3 μmol, 40.3%) as a white solid. Calc'd for C₅₈H₇₃C₁₂F3N₁₀O₂₀S: 1388.41, found [M+H−TFA]*: 1273.4

### Example 99: (R)-2,2′,2″-(10-(20-(3-(1-(3,5-dichlorophenyl)-3-(3,3-dimethylmorpholine-4carbonyl)-7-methoxy-1,4-dihydrochromeno|4,3-c]pyrazol-8-yl) phenoxy)-2,5-dioxo-4-(sulfomethyl)-9,12,15,18-tetraoxa-3,6-diazaicosyl)-1,4,7,10-tetraazacyclododecane-1,4,7-triyl)triacetic acid--2,2,2-trifluoroacetic acid (1/1) (Compound 70)

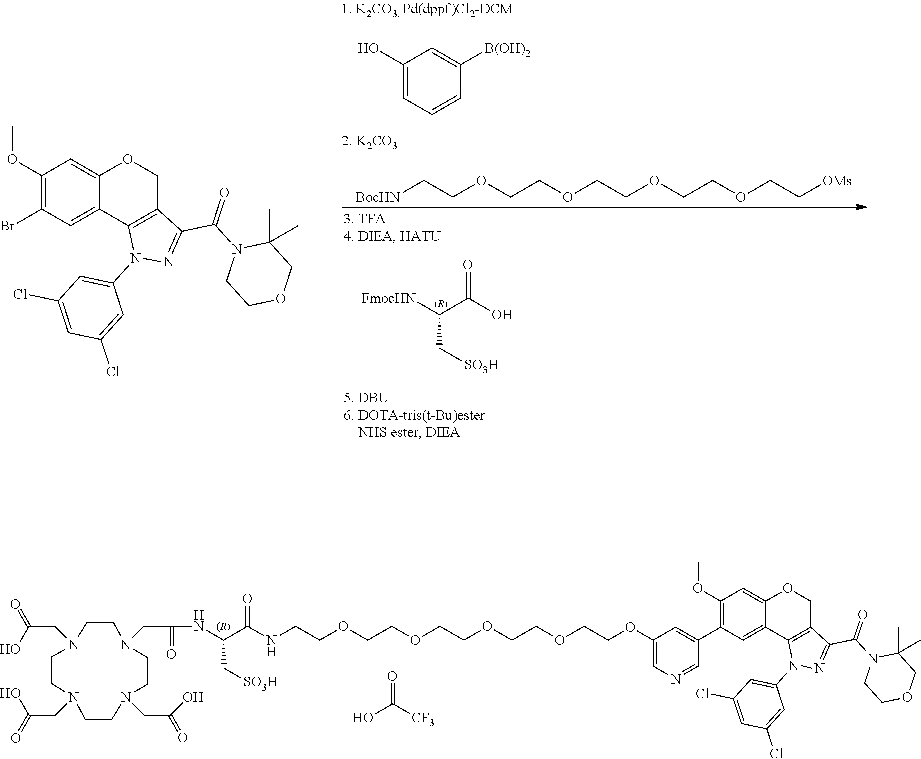

Step 1: Into a 8-mL vial, under an inert atmosphere of nitrogen, was placed a mixture of (8-bromo-1-(3,5-dichlorophenyl)-7-methoxy-1,4-dihydrochromeno[4,3-c]pyrazol-3-yl) (3,3-dimethylmorpholino) methanone (200 mg, 1 Eq, 353 μmol), (3-hydroxyphenyl) boronic acid (58 mg, 1.2 Eq, 0.42 mmol), K₂CO₃ (146 mg, 3.00 Eq, 1.06 mmol), [1,1′-Bis(diphenylphosphino) ferrocene]dichloropalladium (II) Complex with dichloromethane (15 mg, 0.052 Eq, 18 μmol), 1,4-dioxane (2 mL) and H₂O(0.4 mL). The reaction mixture was stirred at 100° C. for 2 hours. The mixture was directly purified by MPLC to provide (1-(3,5-dichlorophenyl)-8-(3-hydroxyphenyl)-7-methoxy-1,4-dihydrochromeno[4,3-c]pyrazol-3-yl) (3,3-dimethylmorpholino) methanone (155 mg, 267 μmol, 75.7%) as a white solid. [M+H]=580.2, 582.2.

Steps 2-6. (R)-2,2′,2″-(10-(20-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy)-2,5-dioxo-4-(sulfomethyl)-9,12, 15, 18-tetraoxa-3,6-diazaicosyl)-1,4,7,10-tetraazacyclododecane-1,4,7-triyl)triacetic acid--2,2,2-trifluoroacetic acid (1/1)_{w} as synthesized from (1-(3,5-dichlorophenyl)-8-(3-hydroxyphenyl)-7-methoxy-1,4-dihydrochromeno[4,3-c]pyrazol-3-yl) (3,3-dimethylmorpholino) methanone in a manner similar to Example 97, Steps 1-5 to provide the product (44 mg, 30 μmol, 38%) as a white solid. Calc'd for C₆₁H₈₀C₁₂F3N₉O₂₂S: 1449.45, found [M+H−TFA]*: 1336.7, 1338.6.

### Example 100: (R)-2,2′,2″-(10-(20-((3-(1-(3,5-dichlorophenyl)-3-(3,3-dimethylmorpholine-4carbonyl)-7-methoxy-1,4-dihydrochromeno|4,3-c]pyrazol-8-yl)phenyl)amino)-2,5-dioxo-4-(sulfomethyl)-9,12,15,18-tetraoxa-3,6-diazaicosyl)-1,4,7,10-tetraazacyclododecane-1,4,7-triyl)triacetic acid--2,2,2-trifluoroacetic acid (1/1) (Compound 71)

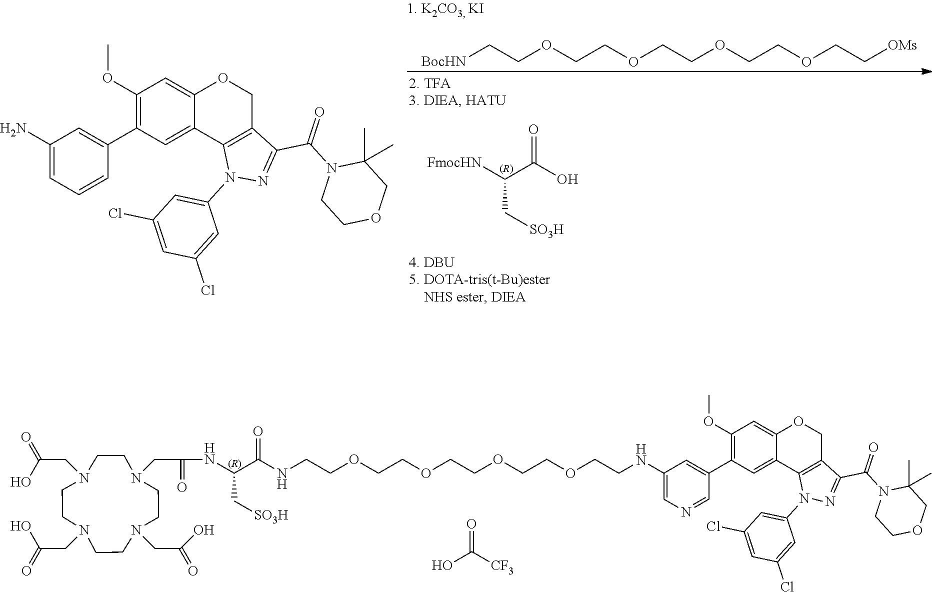

Steps 1-5: (R)-2,2′,2″-(10-(20-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-2,5-dioxo-4-(sulfomethyl)-9,12, 15, 18-tetraoxa-3,6-diazaicosyl)-1,4,7,10-tetraazacyclododecane-1,4,7-triyl)triacetic acid--2,2,2-trifluoroacetic acid (1/1)_{w} as synthesized from 2,2-dimethyl-4-oxo-3,8,11, 14, 17-pentaoxa-5-azanonadecan-19-yl methanesulfonate (181 mg, 1.20 Eq, 436 μmol) and (8-(3-aminophenyl)-1-(3,5-dichlorophenyl)-7-methoxy-1,4-dihydrochromeno[4,3-c]pyrazol-3-yl) (3,3-dimethylmorpholino) methanone in a manner similar to Example 97, Steps 1-5 to provide the product (46.3 mg, 31.9 μmol, 36%) as an off-white solid. Calc'd for C₆₁H₈₁C₁₂F3N₁₀O₂₁S: 1448.46, found [M+H−TFA]⁺: 1335.7.

### Example 101: (R)-2,2′,2″-(10-(1-(5-(1-(3,5-dichlorophenyl)-3-(3,3-dimethylmorpholine-4carbonyl)-7-methoxy-1,4-dihydrochromeno|4,3-c|pyrazol-8-yl) pyridin-3-yl)-15,18-dioxo-16-(sulfomethyl)-2,5,8,11-tetraoxa-14,17-diazanonadecan-19-yl)-1,4,7,10-tetraazacyclododecane-1,4,7-triyl)triacetic acid (Compound 72)

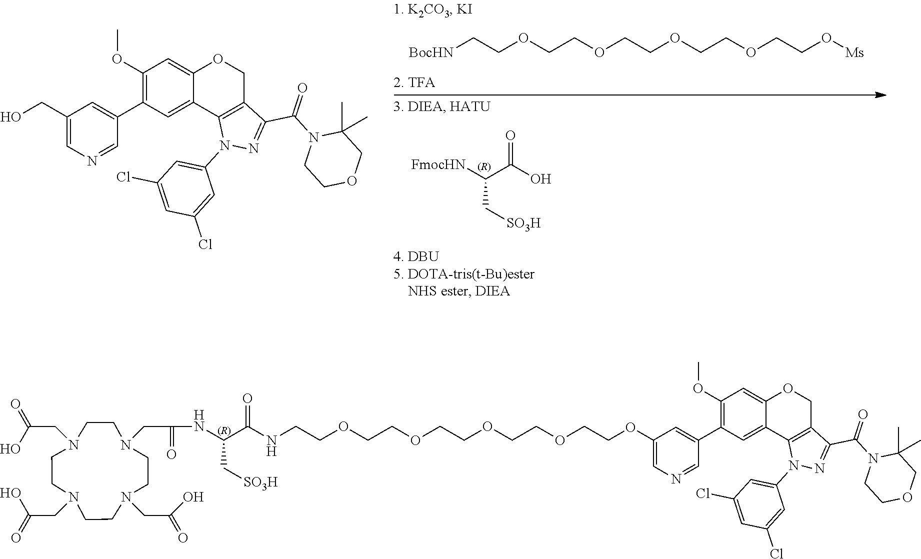

Steps 1-5. (R)-2,2′,2″-(10-(1-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)-15, 18-dioxo-16-(sulfomethyl)-2,5,8,11-tetraoxa-14, 17-diazanonadecan-19-yl)-1,4,7,10-tetraazacyclododecane-1,4,7-triyl)triacetic acid was synthesized from 2,2-dimethyl-4-oxo-3,8, 11,14-tetraoxa-5-azahexadecan-16-yl methanesulfonate (110 mg, 1.1 Eq, 296 μmol) and (1-(3,5-dichlorophenyl)-8-(5-(hydroxymethyl)pyridin-3-yl)-7-methoxy-1,4-dihydrochromeno[4,3-c]pyrazol-3-yl) (3,3-dimethylmorpholino) methanone (160 mg, 0.998 Eq, 269 μmol) in a manner similar to Example 97, Steps 1-5 to provide the product (23.3 mg, 17.5 μmol, 32%) as a white solid. Calc'd for C₅₇H₇₆C₁₂N₁₀O₁₉S: 1306.44, found [M+H]: 1307.5.

### Example 102: (R)-2,2′,2″-(10-(2-((3-((1-((3-(3-(1-(3,5-dichlorophenyl)-3-(3,3dimethylmorpholine-4-carbonyl)-7-methoxy-1,4-dihydrochromeno|4,3-c]pyrazol-8-yl) phenoxy) propyl)amino)-5-guanidino-1-oxopentan-2-yl)amino)-3-oxopropyl)amino)-2-oxoethyl)-1,4,7,10-tetraazacyclododecane-1,4,7-triyl)triacetic acid--2,2,2-trifluoroacetic acid (1/1) (Compound 73)

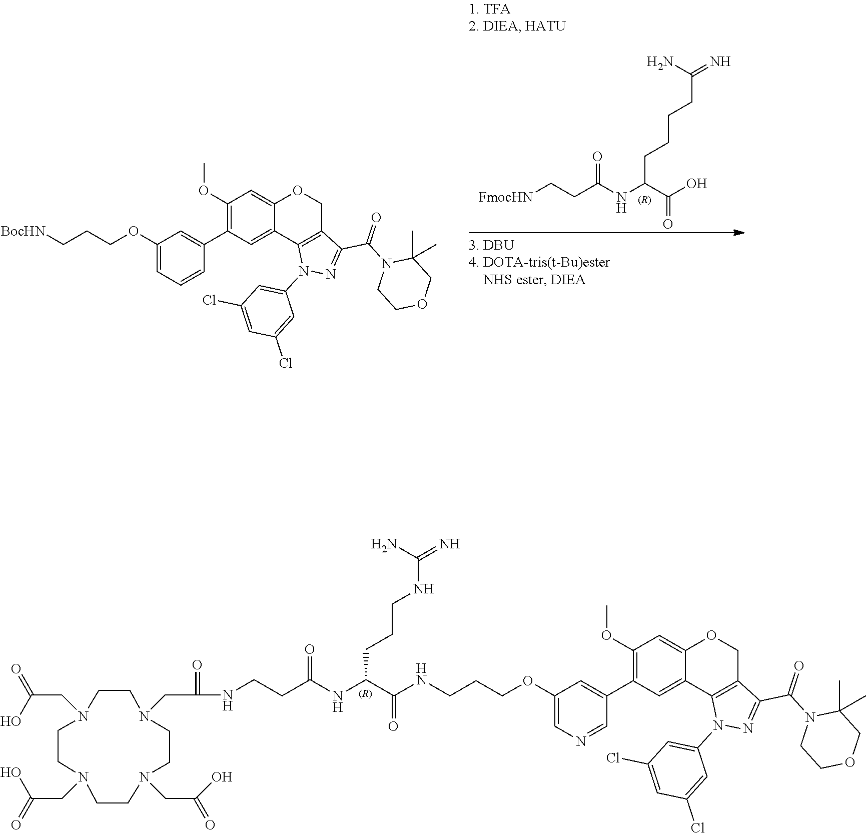

Steps 1-4: (R)-2,2′,2″-(10-(2-((3-((1-((3-(3-(1-(3,5-dichlorophenyl)-3-(3,3-dimethylmorpholine-4-carbonyl)-7-methoxy-1,4-dihydrochromeno[4,3-c]pyrazol-8-y1) phenoxy) propyl)amino)-5-guanidino-1-oxopentan-2-yl)amino)-3-oxopropyl)amino)-2-oxoethyl)-1,4,7,10-tetraazacyclododecane-1,4,7-triyl)triacetic acid--2,2,2-trifluoroacetic acid (1/1)_{w} as synthesized from tert-butyl(3-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy) propyl) carbamate in a manner similar to Example 97, Steps 1-4, to provide the product (40 mg, 29 μmol, 63%) as a white solid. Calc'd for C₆₀H₇₈C₁₂F3N₁₃O₁₆:1363.50, found [M+H−TFA]⁺: 1250.6, 1252.6.

### Example 103:2,2′,2″-(10-((4R,13R)-13-(2-carboxyethyl)-18-(3-(1-(3,5-dichlorophenyl)-3(3,3-dimethylmorpholine-4-carbonyl)-7-methoxy-1,4-dihydrochromeno[4,3-c]pyrazol-8-yl) phenoxy)-2,5,14-trioxo-4-(sulfomethyl)-9-oxa-3,6,12,15-tetraazaoctadecyl)-1,4,7,10-tetraazacyclododecane-1,4,7-triyl)triacetic acid--2,2,2-trifluoroacetic acid (1/1) (Compound 74)

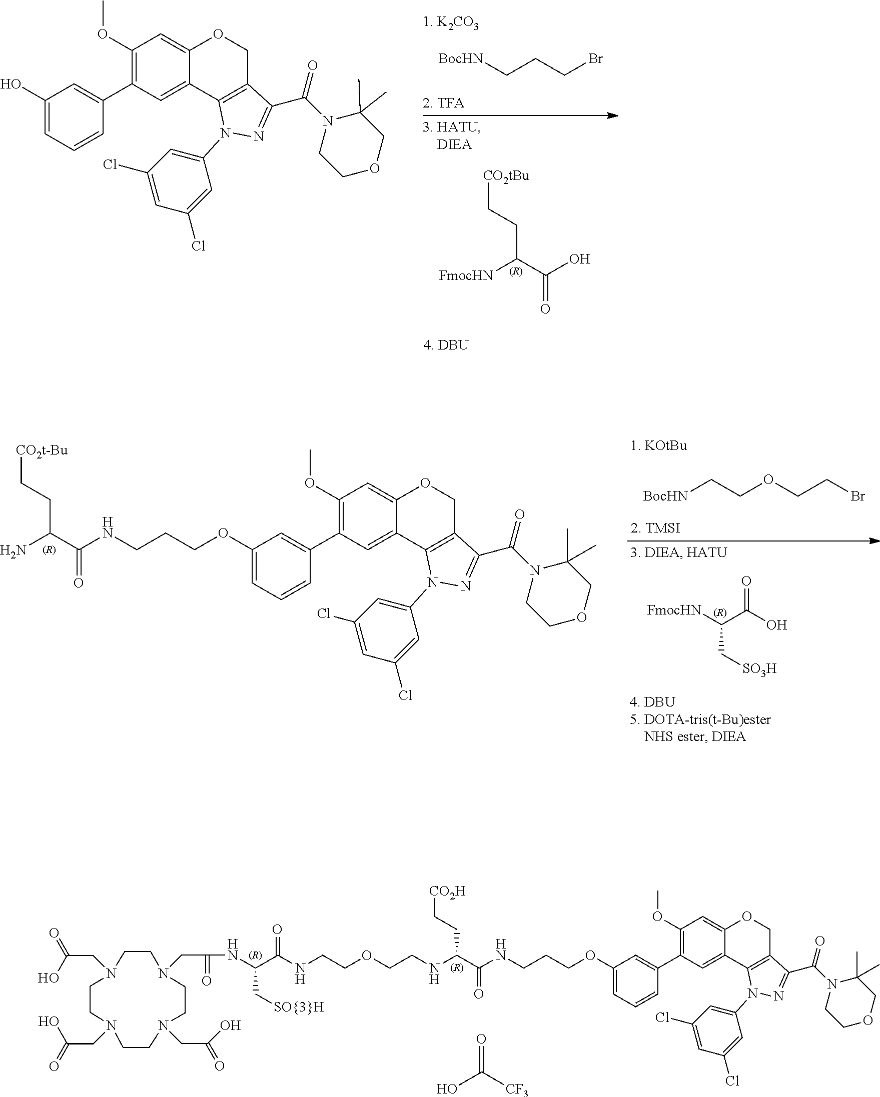

Step 1: Into a 40-mL vial, was placed a mixture of (1-(3,5-dichlorophenyl)-8-(3-hydroxyphenyl)-7-methoxy-1,4-dihydrochromeno[4,3-c]pyrazol-3-yl) (3,3-dimethylmorpholino) methanone (450 mg, 1 Eq, 775 μmol), tert-butyl(3-bromopropyl) carbamate (369 mg, 2.00 Eq, 1.55 mmol), K₂CO₃ (321 mg, 3.00 Eq, 2.32 mmol) and DMF (5 mL). The reaction mixture was stirred at 100° C. for 2 hours. The mixture was diluted with 100 mL of water, extracted with EtOAc (50 mL×3), then the combined organic layers were washed with water (50 mL×2), brine (50 mL), dried over anhydrous Na₂SO₄ and concentrated under reduced pressure. The crude product was purified by MPLC to provide tert-butyl(3-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy) propyl) carbamate (440 mg, 596 μmol, 76.9%) as a white solid. [M+Na]=759.1, 761.1.

Step 2-4: tert-butyl(R)-4-amino-5-((3-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy) propyl)amino)-5-oxopentanoate was synthesized from tert-butyl(3-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy) propyl) carbamate in a manner similar to Example 97, Steps 2-4 to provide the product (100 mg, 122 μmol, 90.7%), which was used directly in the next step without purification. [M+H]=822.1, 824.1.

Step 5: Into an 8-mL vial, was placed a mixture of tert-butyl(R)-4-amino-5-((3-(3-(1-(3-chlorophenyl)-3-(3,3-dimethylmorpholine-4-carbonyl)-7-methoxy-1,4-dihydrochromeno[4,3-c]pyrazol-8-yl) phenoxy) propyl)amino)-5-oxopentanoate (70 mg, 1 Eq, 89 μmol), tert-butyl(2-(2-bromoethoxy)ethyl) carbamate (48 mg, 2.0 Eq, 0.18 mmol), KOtBu (30 mg, 3.0 Eq, 0.27 mmol) and DMF (5 mL). The reaction mixture was stirred at 80° C. for 1 hour. The mixture was directly purified by MPLC to provide tert-butyl(R)-12-((3-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy) propyl) carbamoyl)-2,2-dimethyl-4-oxo-3,8-dioxa-5,11-diazapentadecan-15-oate (21 mg, 21 μmol, 23%) as a white solid. [M+H]=1009.4, 1011.4.

Step 6: Into an 8-mL vial, was placed a mixture of tert-butyl(R)-12-((3-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy) propyl) carbamoyl)-2,2-dimethyl-4-oxo-3,8-dioxa-5,11-diazapentadecan-15-oate (18 mg, 1 Eq, 18 μmol) and MeCN (1 mL), to which was added TMS-I (29 mg, 20 μL, 8.1 Eq, 0.14 mmol). The reaction mixture was stirred at 25° C. for 1 hour, then a saturated aqueous solution of sodium bicarbonate was added to adjust the pH to 9. The crude product was purified by Prep-HPLC to provide (R)-4-((2-(2-aminoethoxy)ethyl)amino)-5-((3-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy) propyl)amino)-5-oxopentanoic acid (6.1 mg, 7.1 μmol, 40%) as a white solid. [M+Na]=875.2, 877.2.

Steps 7-9: 2,2′,2″-(10-((4R, 13R)-13-(2-carboxyethyl)-18-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy)-2,5, 14-trioxo-4-(sulfomethyl)-9-oxa-3,6, 12, 15-tetraazaoctadecyl)-1,4,7,10-tetraazacyclododecane-1,4,7-triyl)triacetic acid--2,2,2-trifluoroacetic acid (1/1)_{w} as synthesized from (R)-4-((2-(2-aminoethoxy)ethyl)amino)-5-((3-(3-(1-(3,5-dichlorophenyl)-3-(3,3-dimethylmorpholine-4-carbonyl)-7-methoxy-1,4-dihydrochromeno[4,3-c]pyrazol-8-yl) phenoxy) propyl)amino)-5-oxopentanoic acid in a manner similar to Example 97, Steps 3-5 to provide the product (1.7 mg, 1.1 μmol, 25%) as a white solid. Calc'd for C₆₃H₈₂C₁₂F3N₁₁O₂₂S: 1503.36, found [M+H−TFA]]: 1390.6, 1392.6.

### Example 104: (R)-2,2′,2″-(10-(20-(3-(1-(3,5-dichlorophenyl)-3-(2,2-dimethyl-5oxopiperazine-1-carbonyl)-7-methoxy-1,4-dihydrochromeno|4,3-c]pyrazol-8-yl) phenoxy)-2,5-dioxo-4-(sulfomethyl)-9,12,15,18-tetraoxa-3,6-diazaicosyl)-1,4,7,10-tetraazacyclododecane-1,4,7-triyl)triacetic acid (Compound 75)

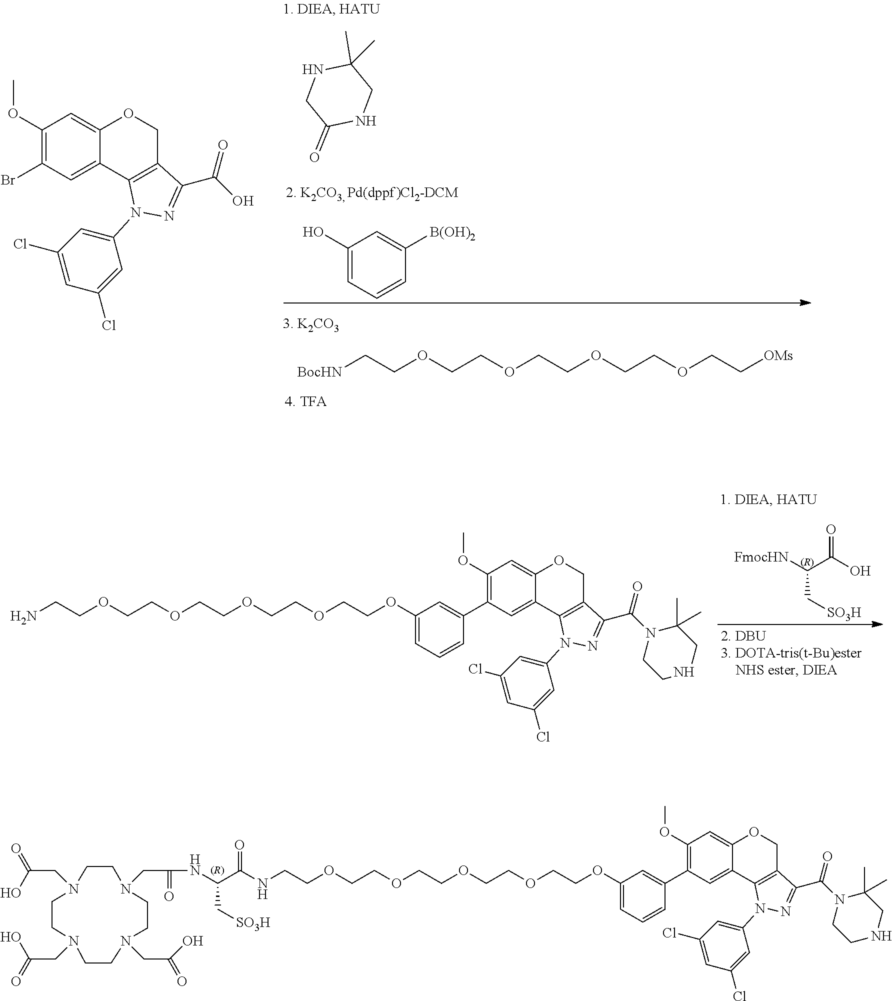

Step 1: Into a 40-mL vial, was placed a mixture of 8-bromo-1-(3,5-dichlorophenyl)-7-methoxy-1,4-dihydrochromeno[4,3-c]pyrazole-3-carboxylic acid (140 mg, 1 Eq, 298 μmol), HATU (170 mg, 1.50 Eq, 447 μmol), DIEA (115 mg, 155 μL, 2.99 Eq, 890 μmol) and DMF (2 mL). The reaction mixture was stirred at 20° C. for 10 minutes, then 5,5-dimethylpiperazin-2-one (456 mg, 11.9 Eq, 3.56 mmol)_{w} as added and the reaction mixture was stirred at 25° C. for 1 hour. The mixture was directly purified by MPLC to provide 4-(8-bromo-1-(3,5-dichlorophenyl)-7-methoxy-1,4-dihydrochromeno[4,3-c]pyrazole-3-carbonyl)-5,5-dimethylpiperazin-2-one (120 mg, 207 μmol, 69.4%) as a white solid. [M+H]=578.9, 580.9.

Step 2-7. (R)-2,2′,2″-(10-(20-(3-(1-(3,5-dichlorophenyl)-3-(2,2-dimethyl-5-oxopiperazine-1-carbonyl)-7-methoxy-1,4-dihydrochromeno[4,3-c]pyrazol-8-yl) phenoxy)-2,5-dioxo-4-(sulfomethyl)-9, 12, 15, 18-tetraoxa-3,6-diazaicosyl)-1,4, 7, 10-tetraazacyclododecane-1,4,7-triyl)triacetic acid was synthesized from 4-(8-bromo-1-(3,5-dichlorophenyl)-7-methoxy-1,4-dihydrochromeno[4,3-c]pyrazole-3-carbonyl)-5,5-dimethylpiperazin-2-one in a manner similar to Example 99, Steps 1-6 to provide the product (16.6 mg, 12.3 μmol, 30%) as a white solid. Calc'd for C₅₉H₇₈C₁₂F3N₁₀O₂₀S: 1348.45, found [M+H]⁺: 1349.6, 1351.6.

### Example 105: (R)-2,2′,2″-(10-(20-((5-(3-(4-(cyclobutanecarbonyl)-1,4-diazepane-1carbonyl)-7-methoxy-1-(thiophen-2-yl)-1,4-dihydrochromeno[4,3-c]pyrazol-8-yl) pyridin-3-yl)oxy)-2,5-dioxo-4-(sulfomethyl)-9,12,15,18-tetraoxa-3,6-diazaicosyl)-1,4,7,10-tetraazacyclododecane-1,4,7-triyl)triacetic acid (Compound 76)

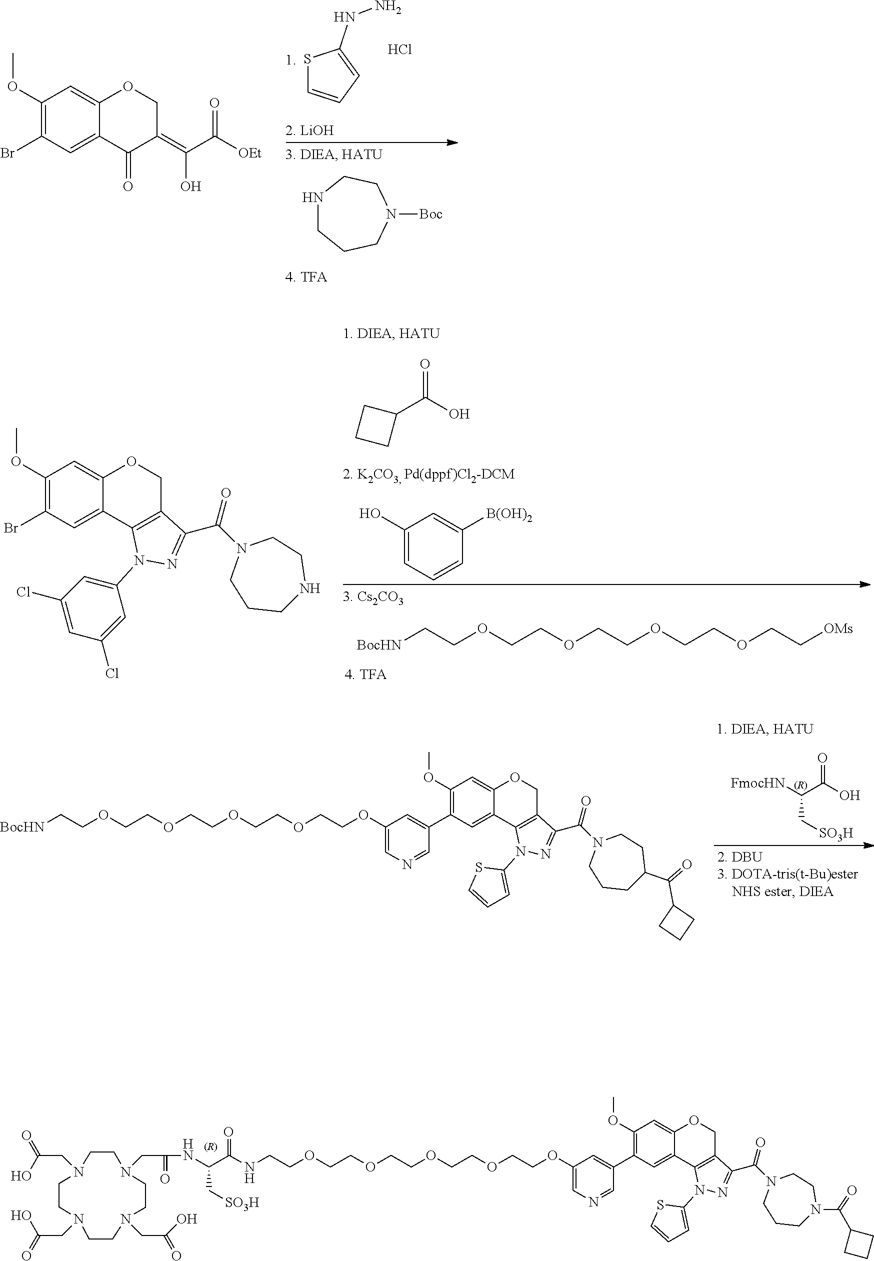

Step 1: Into a 100-mL round bottom flask, was placed a mixture of ethyl (Z)-2-(6-bromo-7-methoxy-4-oxochroman-3-ylidene)-2-hydroxyacetate (1.8 g, 1 Eq, 5.0 mmol), thiophen-2-ylhydrazine hydrochloride (1.5 g, 2.0 Eq, 10 mmol) and t-BuOH (20 mL), to which was added AcOH (10 mL). The reaction mixture was stirred at 100° C. for 3 hours. The mixture was concentrated under reduced pressure. The crude product was washed by PE to provide (R)-17-amino-1-((5-(3-(4-(cyclobutanecarbonyl)-1,4-diazepane-1-carbonyl)-7-methoxy-1-(thiophen-2-y1)-1,4-dihydrochromeno[4,3-c]pyrazol-8-yl) pyridin-3-yl)oxy)-16-oxo-3,6,9,12-tetraoxa-15-azaoctadecane-18-sulfonic acid (85 mg, 62 μmol, 86%) as a white solid.

Step 2: (R)-17-amino-1-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy)-16-oxo-3,6,9,12-tetraoxa-15-azaoctadecane-18-sulfonic acid was treated with LiOH in a manner similar to Example 78, Step 2 to provide 8-bromo-7-methoxy-1-(thiophen-2-yl)-1,4-dihydrochromeno[4,3-c]pyrazole-3-carboxylic acid (1.26 g, 3.09 mmol, 92.6%) as an off-white solid. [M+H]=406.4, 408.4.

Step 3: 8-bromo-7-methoxy-1-(thiophen-2-yl)-1,4-dihydrochromeno[4,3-c]pyrazole-3-carboxylic acid was treated with 8-bromo-7-methoxy-1-(thiophen-2-yl)-1,4-dihydrochromeno[4,3-c]pyrazole-3-carboxylic acid (1.26 g, 1 Eq, 3.09 mmol), HATU and DIEA in a manner similar to Example 78, Step 6 to provide tert-butyl 4-(8-bromo-7-methoxy-1-(thiophen-2-yl)-1,4-dihydrochromeno[4,3-c]pyrazole-3-carbonyl)-1,4-diazepane-1-carboxylate (950 mg, 1.61 mmol, 52.1%) as a yellow oil. [M+H]=589.0, 591.0.

Step 4: tert-Butyl 4-(8-bromo-7-methoxy-1-(thiophen-2-yl)-1,4-dihydrochromeno[4,3-c]pyrazole-3-carbonyl)-1,4-diazepane-1-carboxylate was treated with TFA in a manner similar to Example 79, Step 8 to provide (8-bromo-7-methoxy-1-(thiophen-2-yl)-1,4-dihydrochromeno[4,3-c]pyrazol-3-yl) (1,4-diazepan-1-yl) methanone (750 mg, 1.53 mmol, 95.1%) as a white solid. [M+H]=489.2, 491.2.

Step 5: (8-bromo-7-methoxy-1-(thiophen-2-yl)-1,4-dihydrochromeno[4,3-c]pyrazol-3-yl) (1,4-diazepan-1-yl) methanone was treated with cyclobutanecarboxylic acid, HATU and DIEA in a manner similar to Example 78, Step 6 to provide (8-bromo-7-methoxy-1-(thiophen-2-yl)-1,4-dihydrochromeno[4,3-c]pyrazol-3-yl) (4-(cyclobutanecarbonyl)-1,4-diazepan-1-yl) methanone (700 mg, 1.22 mmol, 79.9%) as a yellow oil. [M+H]=571.3, 573.3

Step 6: (8-bromo-7-methoxy-1-(thiophen-2-yl)-1,4-dihydrochromeno[4,3-c]pyrazol-3-yl) (4-(cyclobutanecarbonyl)-1,4-diazepan-1-yl) methanone was treated with (5-hydroxypyridin-3-yl) boronic acid in a manner similar to Example 99, Step 1 to provide (4-(cyclobutanecarbonyl)-1,4-diazepan-1-yl) (8-(5-hydroxypyridin-3-yl)-7-methoxy-1-(thiophen-2-yl)-1,4-dihydrochromeno[4,3-c]pyrazol-3-yl) methanone (590 mg, 1.01 mmol, 82.2%) as an off-white solid. [M+H]=586.1.

Steps 7-11: (R)-2,2′,2″-(10-(20-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy)-2,5-dioxo-4-(sulfomethyl)-9,12,15, 18-tetraoxa-3,6-diazaicosyl)-1,4,7,10-tetraazacyclododecane-1,4,7-triyl)triacetic acid was synthesized from (4-(cyclobutanecarbonyl)-1,4-diazepan-1-yl) (8-(5-hydroxypyridin-3-yl)-7-methoxy-1-(thiophen-2-yl)-1,4-dihydrochromeno[4,3-c]pyrazol-3-yl) methanone in a manner similar to Example 97, Steps 1-5 to provide the product (61.2 mg, 45.6 μmol, 73%) as a white solid. Calc'd for C₆₀H₈₃N₁₁O₂₀S2:1341. 52, found [M+H−TFA]⁺: 1342.6.

### Example 106: (R)-2,2′,2″-(10-(2-((3-((4-carboxy-1-((3-((5-(3-(4-(cyclobutanecarbonyl)-1,4diazepane-1-carbonyl)-7-methoxy-1-(thiophen-2-yl)-1,4-dihydrochromeno|4,3-c]pyrazol-8-yl) pyridin-3-yl)oxy) propyl)amino)-1-oxobutan-2-yl)amino)-3-oxopropyl)amino)-2-oxoethyl)-1,4,7,10-tetraazacyclododecane-1,4,7-triyl)triacetic acid (Compound 77)

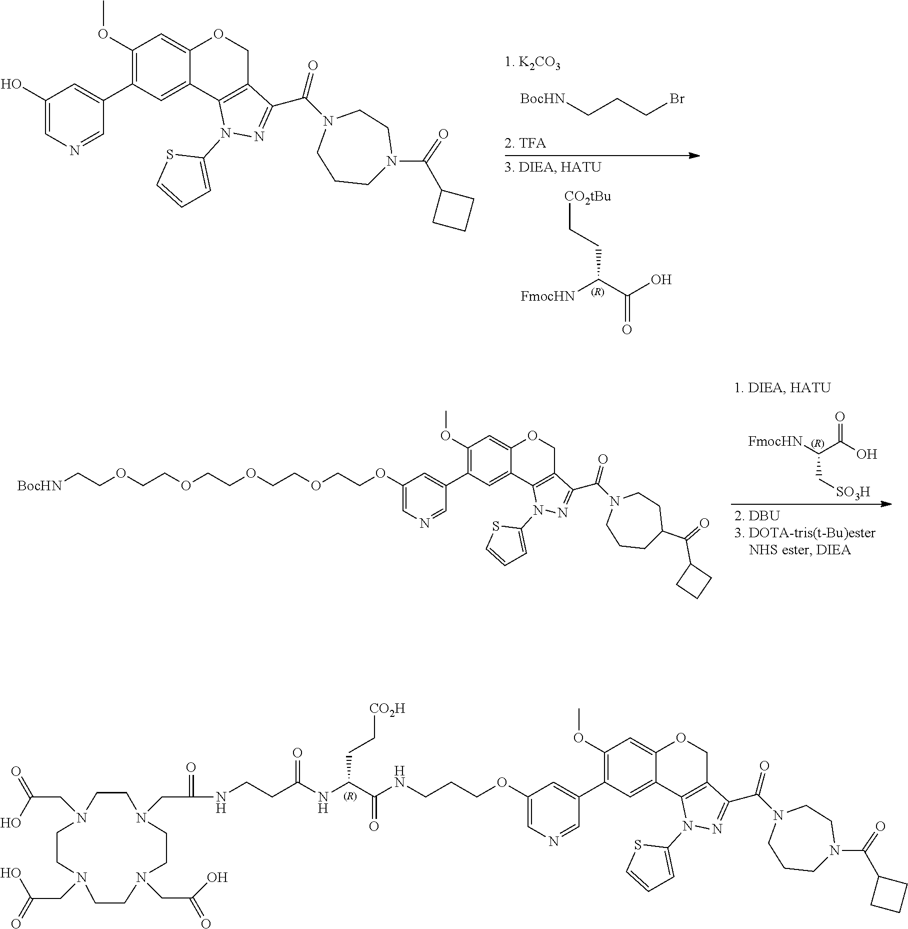

Step 1: Into a 8-mL vial, was placed a mixture of (4-(cyclobutanecarbonyl)-1,4-diazepan-1-yl) (8-(5-hydroxypyridin-3-yl)-7-methoxy-1-(thiophen-2-yl)-1,4-dihydrochromeno[4,3-c]pyrazol-3-yl) methanone (280 mg, 1 Eq, 478 μmol), tert-butyl(3-bromopropyl) carbamate (228 mg, 2.00 Eq, 957 μmol), K₂CO₃ (198 mg, 3.00 Eq, 1.43 mmol) and DMF (3 mL). The reaction mixture was stirred at 100° C. for 2 hours. The mixture was directly purified by MPLC to provide tert-butyl(3-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy) propyl) carbamate (165 mg, 222 μmol, 46.5%) as a yellow solid. [M+H]=743.6.

Steps 2-4: (8-(5-(3-aminopropoxy)pyridin-3-yl)-7-methoxy-1-(thiophen-2-yl)-1,4-dihydrochromeno[4,3-c]pyrazol-3-yl) (4-(cyclobutanecarbonyl)-1,4-diazepan-1-yl) methanone was synthesized from tert-butyl(3-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy) propyl) carbamate in a manner similar to Example 97, Steps 2-4 to provide the product tert-butyl(R)-4-amino-5-((3-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy) propyl)amino)-5-oxopentanoate (50 mg, 60 μmol, 85%) as a yellow solid. [M+H]=828.4.

Step 5: Into an 8-mL vial, was placed a mixture of tert-butyl(R)-4-amino-5-((3-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy) propyl)amino)-5-oxopentanoate (50 mg, 1 Eq, 60 μmol), 2,5-dioxopyrrolidin-1-yl 3-((tert-butoxycarbonyl)amino) propanoate (35 mg, 2.0 Eq, 0.12 mmol), DIPEA (40 mg, 54 μL, 5.1 Eq, 0.31 mmol) and DMF (1 mL). The reaction mixture was stirred at 25° C. for 1 hour. The mixture was directly purified by MPLC to provide tert-butyl(R)-4-(3-((tert-butoxycarbonyl)amino) propanamido)-5-((3-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy) propyl)amino)-5-oxopentanoate (45 mg, 45 μmol, 75%) as a yellow oil. [M+H]=999.9.

Steps 6-7: (R)-2,2′,2″-(10-(2-((3-((4-carboxy-1-((3-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy) propyl)amino)-1-oxobutan-2-yl)amino)-3-oxopropyl)amino)-2-oxoethyl)-1,4,7,10-tetraazacyclododecane-1,4,7-triyl)triacetic acid was synthesized from tert-butyl(R)-4-(3-((tert-butoxycarbonyl)amino) propanamido)-5-((3-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy) propyl)amino)-5-oxopentanoate in a manner similar to Example 97, Step 2 and 5 to provide the product (47.3 mg, 38.5 μmol, 65%) as an off white solid. Calc'd for C₅₈H₇₆N₁₂O₁₆S: 1228.52, found [M+H]: 1229.7.

### Example 107: (R)-2,2′,2″-(10-(2-((3-((1-((3-((5-(3-(4-(cyclobutanecarbonyl)-1,4-diazepane-1carbonyl)-7-methoxy-1-(thiophen-2-yl)-1,4-dihydrochromeno|4,3-c|pyrazol-8-yl) pyridin-3-yl)oxy) propyl)amino)-5-guanidino-1-oxopentan-2-yl)amino)-3-oxopropyl)amino)-2-oxoethyl)-1,4,7,10-tetraazacyclododecane-1,4,7-triyl)triacetic acid--2,2,2-trifluoroacetic acid (1/1) (Compound 78)

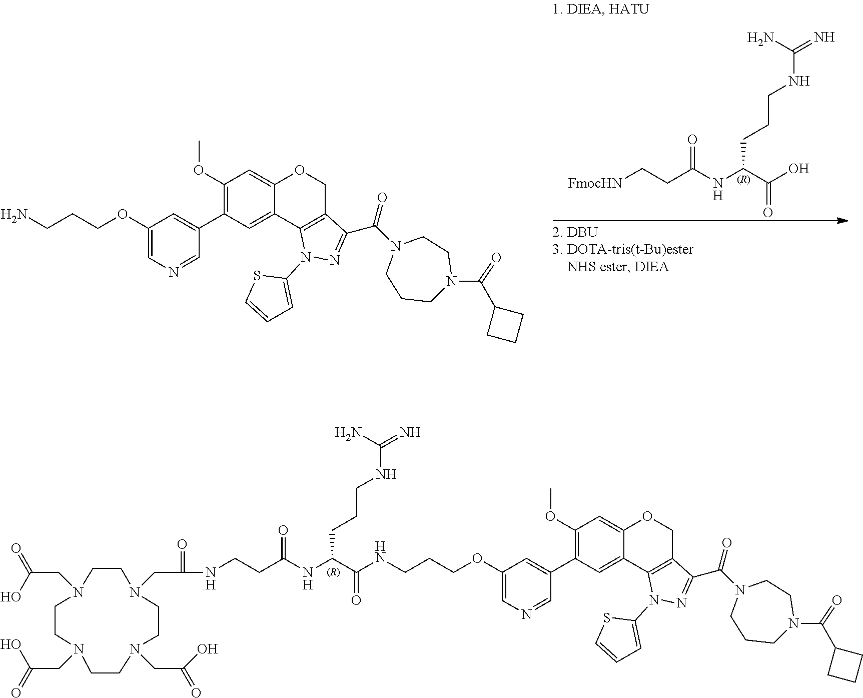

Steps 1-3: (R)-2,2′,2″-(10-(2-((3-((1-((3-((5-(3-(4-(cyclobutanecarbonyl)-1,4-diazepane-1-carbonyl)-7-methoxy-1-(thiophen-2-yl)-1,4-dihydrochromeno[4,3-c]pyrazol-8-yl) pyridin-3-yl)oxy) propyl)amino)-5-guanidino-1-oxopentan-2-yl)amino)-3-oxopropyl)amino)-2-oxoethyl)-1,4,7,10-tetraazacyclododecane-1,4,7-triyl)triacetic acid--2,2,2-trifluoroacetic acid (1/1)_{w} as synthesized from (8-(5-(3-aminopropoxy)pyridin-3-yl)-7-methoxy-1-(thiophen-2-yl)-1,4-dihydrochromeno[4,3-c]pyrazol-3-yl) (4-(cyclobutanecarbonyl)-1,4-diazepan-1-yl) methanone and (3-((((9H-fluoren-9-yl) methoxy) carbonyl)amino) propanoyl)-D-arginine in a manner similar to Example 97, Steps 3-5 to provide the product (37 mg, 27 μmol, 47%) as a white solid. Calc'd for C₆₁H₈₂F3N₁₅O₁₆S: 1369.57, found [M+H−TFA]⁺: 1256.8.

### Example 108: (R)-2,2′,2″-(10-(2-((5-((tert-Butoxycarbonyl)amino)-6-((4-((5-(1-(3,5dichlorophenyl)-3-(3,3-dimethylmorpholine-4-carbonyl)-7-methoxy-1,4-dihydro-chromeno[4,3-c]pyrazol-8-yl) pyridin-3-yl)amino)-4-oxobutyl)amino)-6-oxohexyl)amino)-2-oxoethyl)-1,4,7,10-tetraazacyclododecane-1,4,7-triyl)triacetic acid (Compound 79)

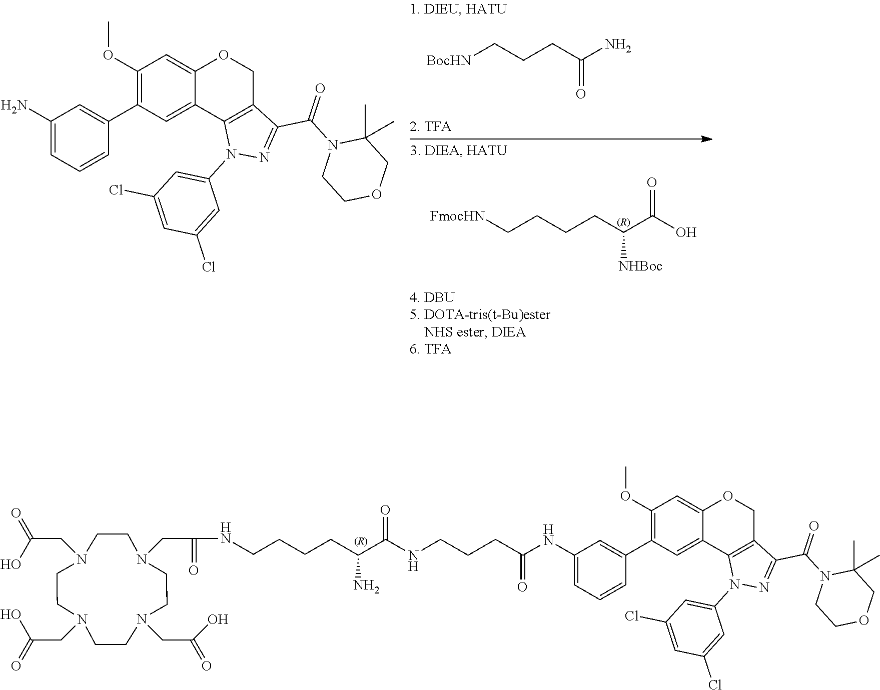

Steps 1-2: 4-amino-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) butanamide was synthesized from 8-(5-aminopyridin-3-yl)-1-(3,5-dichlorophenyl)-7-methoxy-1,4-dihydrochromeno[4,3-c]pyrazol-3-yl) (3,3-dimethylmorpholino) methanone and 4-((tert-butoxycarbonyl)amino) butanoic acid in a manner similar to Example 97, Steps 3-4 to provide the product (180 mg, 0.24 mmol, 100%) as a light yellow oil. [M+H−TFA]=665.3, 667.3.

Steps 3-5: (R)-2,2′,2″-(10-(2-((5-((tert-butoxycarbonyl)amino)-6-((4-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-6-oxohexyl)amino)-2-oxoethyl)-1,4,7,10-tetraazacyclododecane-1,4,7-triyl)triacetic acid was synthesized from N₆-(((9H-fluoren-9-yl) methoxy) carbonyl)-N₂-(tert-butoxycarbonyl)-D-lysine and 4-amino-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) butanamide in a manner similar to Example 97, Steps 3-5 to provide the product as (58 mg, 45 μmol, 72%) as an off-white solid. [M/2+H]=641.2.

Step 6: (R)-2,2′,2″-(10-(2-((5-((tert-Butoxycarbonyl)amino)-6-((4-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-6-oxohexyl)amino)-2-oxoethyl)-1,4,7,10-tetraazacyclododecane-1,4,7-triyl)triacetic acid was treated with TFA in a manner similar to Example 79, Step 8 to provide (R)-2,2′,2″-(10-(2-((5-amino-6-((4-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-6-oxohexyl)amino)-2-oxoethyl)-1,4,7,10-tetraazacyclododecane-1,4,7-triyl)triacetic acid--2,2,2-trifluoroacetic acid (1/1) (56 mg, 41 μmol, 91%) as a light yellow solid. Calc'd for C₅₇H₇₃C₁₂F3N₁₂O₁₅:1292.46, found [M+H−TFA]*:

1179.6, 1181.6.

### Example 109: (R)-2,2′,2″-(10-(21-((5-(1-(3,5-dichlorophenyl)-3-(3,3-dimethylmorpholine-4carbonyl)-7-methoxy-1,4-dihydrochromeno|4,3-c]pyrazol-8-yl) pyridin-3-yl)amino)-2,5,21-trioxo-4-(sulfomethyl)-9,12,15,18-tetraoxa-3,6-diazahenicosyl)-1,4,7,10-tetra-azacyclododecane-1,4,7-triyl)triacetic acid--2,2,2-trifluoroacetic acid (1/1) (Compound 80)

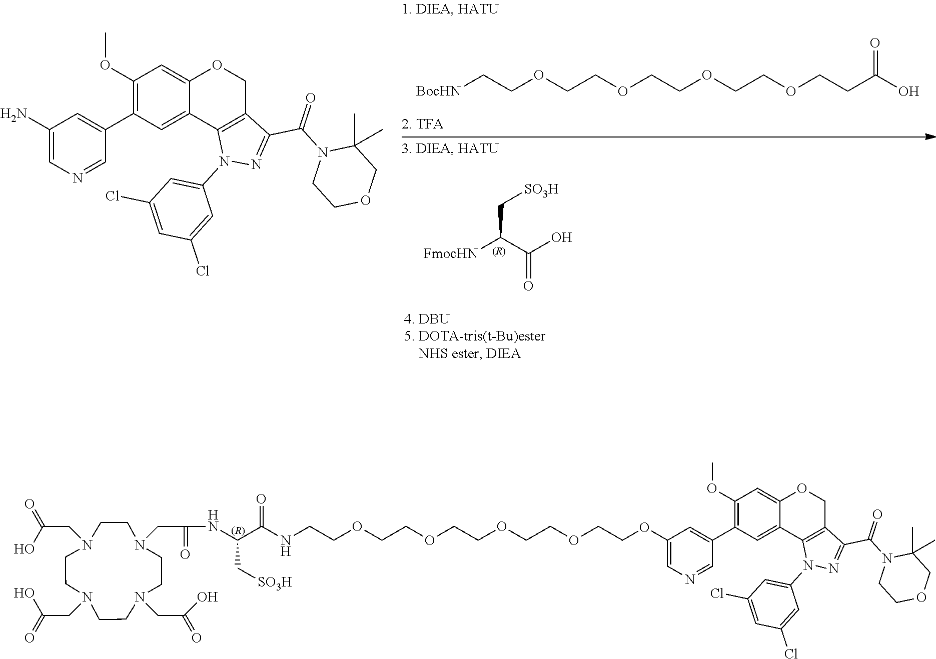

Step 1: (8-(5-aminopyridin-3-yl)-1-(3,5-dichlorophenyl)-7-methoxy-1,4-dihydrochromeno[4,3-c]pyrazol-3-yl) (3,3-dimethylmorpholino) methanone was treated with 2,2-dimethyl-4-oxo-3,8, 11, 14, 17-pentaoxa-5-azaicosan-20-oic acid, HATU and DIEA in a manner similar to Example 78, Step 6 to provide tert-butyl(15-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-15-oxo-3,6,9,12-tetraoxapentadecyl) carbamate (160 mg, 172 μmol, 62.6%) as a yellow oil. [M+H]=927.4, 929.3.

Steps 2-5: (R)-2,2′,2″-(10-(21-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-2,5,21-trioxo-4-(sulfomethyl)-9, 12, 15,18-tetraoxa-3,6-diazahenicosyl)-1,4, 7,10-tetraazacyclododecane-1,4,7-triyl)triacetic acid--2,2,2-trifluoroacetic acid (1/1)_{w} as synthesized from tert-butyl(15-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-15-oxo-3,6,9,12-tetraoxapentadecyl) carbamate in a manner similar to Example 97, Steps 2-5 to provide the product (13.7 mg, 9.26 μmol, 30%) as a white solid. Calc'd for C₆₁H₈₀C₁₂F3N₁₁O₂₂S: 1477.45, found [M+H−TFA]]: 1364.6.

### Example 110: (R)-2,2′,2″-(10-(2-((3-((1-((4-((5-(1-(3,5-dichlorophenyl)-3-(3,3dimethylmorpholine-4-carbonyl)-7-methoxy-1,4-dihydrochromeno|4,3-clpyrazol-8-yl) pyridin-3-yl)amino)-4-oxobutyl)amino)-5-guanidino-1-oxopentan-2-yl)amino)-3-oxopropyl)amino)-2-oxoethyl)-1,4,7,10-tetraazacyclododecane-1,4,7-triyl)triacetic acid--2,2,2-trifluoroacetic acid (1/1) (Compound 81)

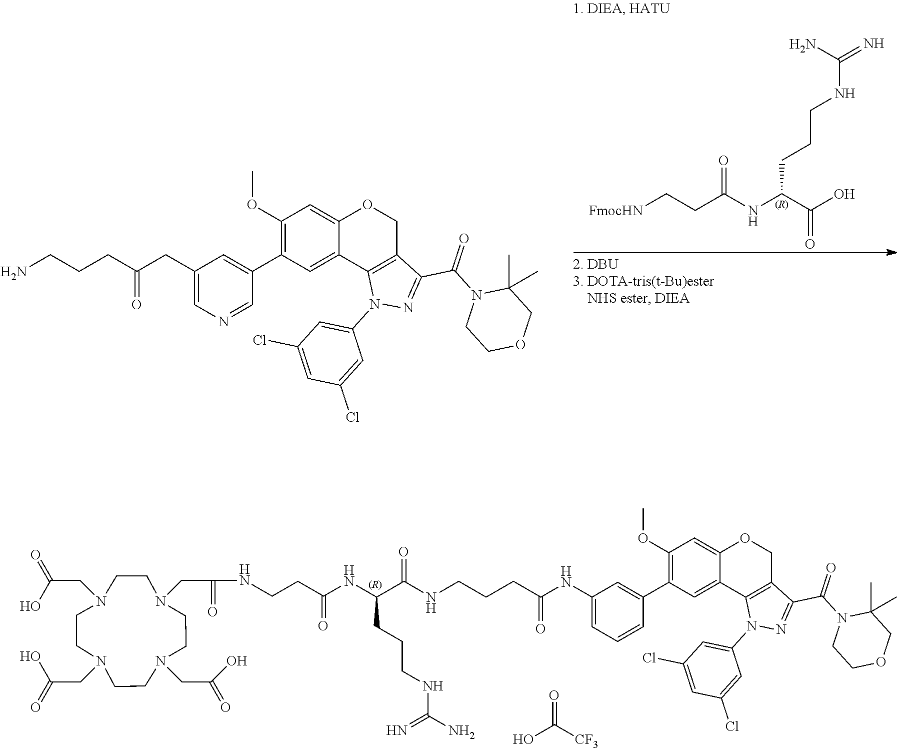

Steps 1-3: (R)-2,2′,2″-(10-(2-((3-((1-((4-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-4-oxobutyl)amino)-5-guanidino-1-oxopentan-2-yl)amino)-3-oxopropyl)amino)-2-oxoethyl)-1,4,7,10-tetraazacyclododecane-1,4,7-triyl)triacetic acid--2,2,2-trifluoroacetic acid (1/1)_{w} as synthesized from 4-amino-N-(5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl) butanamide and (3-((((9H-fluoren-9-yl) methoxy) carbonyl)amino) propanoyl)-D-arginine in a manner similar to Example 97, Steps 2-4 to provide the product (24.2 mg, 17.4 μmol, 28%) as a white solid. Calc'd for C₆₀H₇₈C₁₂F3N₁₅O₁₆:1391.51, found [M+H−TFA]⁺: 1278.7.

### Example 111:2,2′,2″-(10-((R)-8-((2-(2-(2-(((S)-1-((S)-2-(((S)-1-(((S)-1-(((R)-1-carboxy-2mercaptoethyl)amino)-3-(1H-imidazol-4-yl)-1-oxopropan-2-yl)amino)-3-hydroxy-1-oxopropan-2-yl) carbamoyl) pyrrolidin-1-yl)-3-(4-hydroxyphenyl)-1-oxopropan-2-yl)amino)-2-oxoethoxy)ethoxy)ethyl) carbamoyl)-22-((3-(1-(3,5-dichlorophenyl)-3-(3,3-dimethylmorpholine-4-carbonyl)-7-methoxy-1,4-dihydrochromeno|4,3-c]pyrazol-8-y) pheny/)amino)-2,10,19,22-tetraoxo-12,15-dioxa-3,9,18-triazadocosyl)-1,4,7,10-tetraazacyclododecane-1,4,7-triyl)triacetic acid (Compound 82)

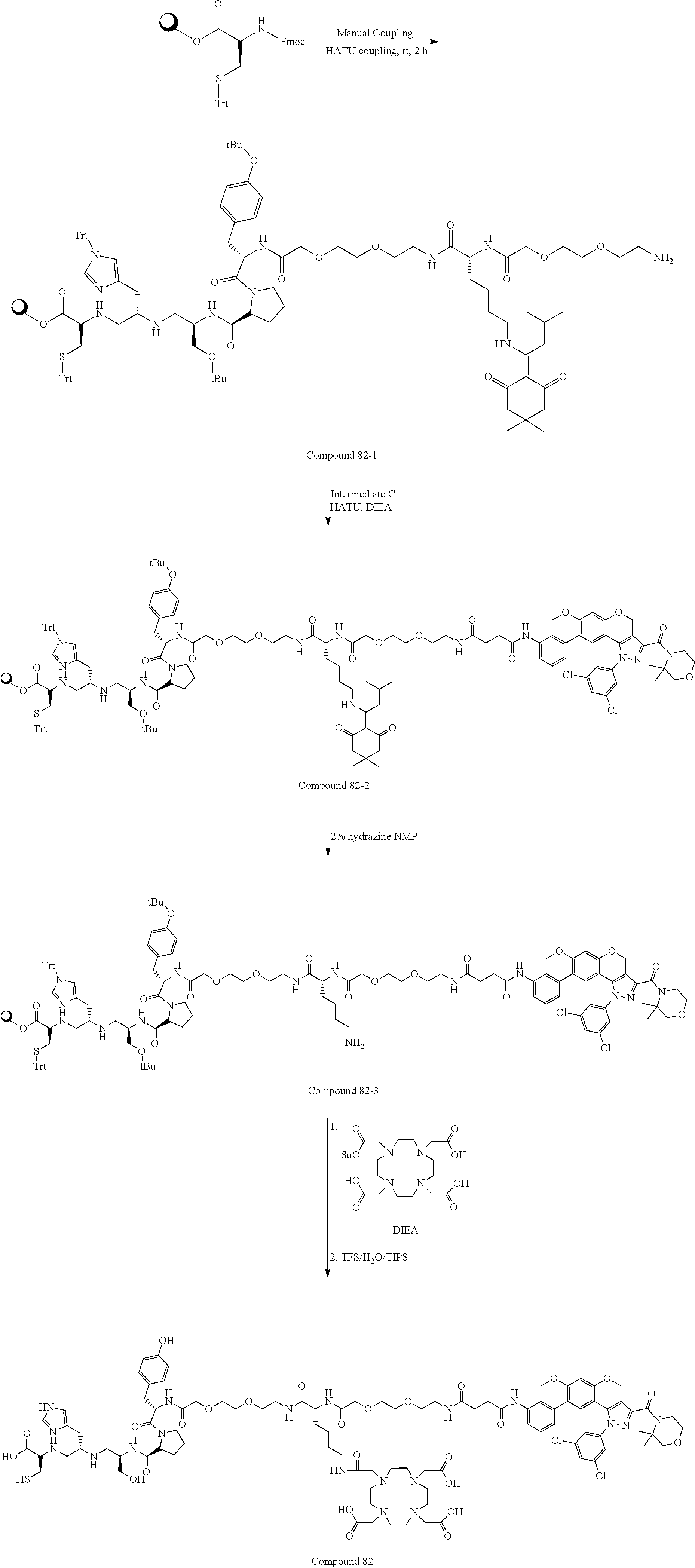

Step 1. Synthesis of Compound 82-1: The linear peptide was prepared by manual method. Resin pre-swelling: The Fmoc Cys (Trt)-2-Chlorotrityl resin (300 mg, 100-200 mesh, 0.647 mmol/g)_{w} as swelled in NMP (6 mL/tube) for 30 min at room temperature under a nitrogen atmosphere. The solvent was then removed. SPPS coupling cycle: A) Fmoc de-protection: the resin (300 mg)_{w} as treated with 20% of piperidine in NMP (6 mL) for 30 min at room temperature under nitrogen atmosphere. The resin was then washed 4 times with NMP (6 mL). B) HATU coupling: the resin (300 mg)_{w} as treated with the mixture of amino acid (2 eq.), HATU (2 eq.) and DIPEA (4 eq.) in NMP (6 mL) for 2 h at room temperature under nitrogen atmosphere. Then, the resin was washed 4 times with NMP (6 mL).

Step 2: Synthesis of Compound 82-2: To the resin (300 mg) in 25 mL reactor were added Intermediate C (2 eq.), HATU (2 eq.) and DIPEA (4 eq.) in NMP (5 mL). After the reaction mixture was incubated for 2 h at room temperature, the solvent was removed and the resin was washed with NMP (6 mL×6).

Step 3. Synthesis of Compound 82-3: Compound 82-2 on resin (300 mg)_{w} as swelled with NMP (6 mL) for 30 min at room temperature. The solvent was then removed. To the resin (300 mg) in 25 mL reactor was added 2% hydrazine in NMP (5 mL) at room temperature under nitrogen atmosphere. The mixture was bubbled for 2 h and filtered, washed with NMP (6 mL×6). The reaction was monitored by LCMS.

Step 4: To the resin (300 mg) in 25 mL reactor were added DOTA-NHS (2 eq.) and DIPEA (6 eq.) in NMP (6 mL). After the reaction mixture was incubated for 2 h at room temperature, the solvent was removed and the resin was washed with NMP (6 mL×6).

Step 5: The resin was treated with the cleavage solution (v v v TFA/H₂O/TIS=37:1:1) for 2 h at room temperature. After filtration, the filtrate was concentrated under reduced pressure. The residue was precipitated with cold ether. After centrifugation, the solid was collected and purified by prep-HPLC. The desired fractions were combined and lyophilized to afford Compound 82 as a white solid (24.7 mg, 6.0%, 95.0% purity). Calc'd for C₉₄H₁₂₅C₁₂N₁₉O₂₈S: 2069.8, found [M+2H]2-: 1036.6.

### Example 112:2,2′,2″-(10-((4R,7R,10R,13R,16R,19R,22R)-23-amino-10,19-bis(2carboxyethyl)-13-(4-(4-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-4-oxobutanamido)butyl)-7,16-bis(3-guanidinopropyl)-4,22-dimethyl-2,5,8,11,14,17,20,23-octaoxo-3,6,9,12,15,18,21-heptaazatricosyl)-1,4,7,10-tetraazacyclododecane-1,4,7-triyl)triacetic acid (Compound 83)

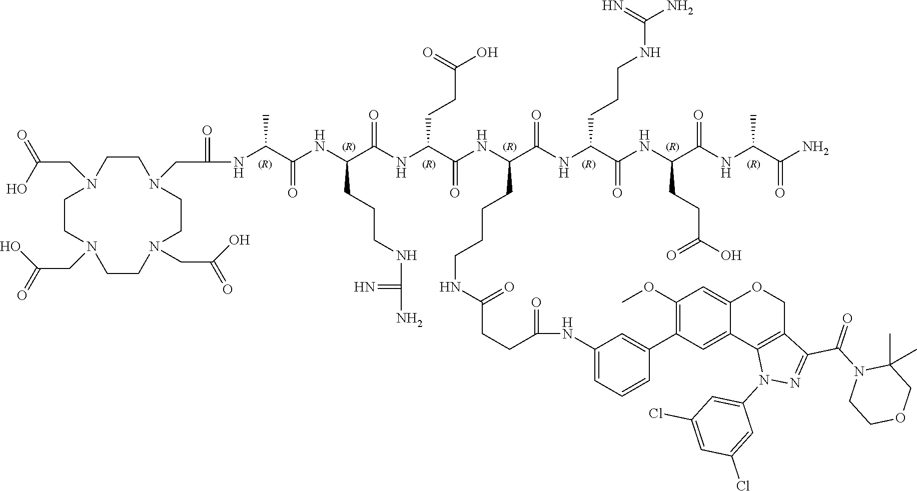

Compound 83 was prepared in a manner similar to that for Example 75. Calc'd for C&H₁₁₉C₁₂N₂₃O₂₄:1903.8, found [M+H]: 1905.3.

### Example 113:2,2′,2″-(10-((4R,7R,10R,13R,16R,19R)-19-(((R)-1-amino-1-oxopropan-2yl) carbamoyl)-10,16-bis(2-carboxyethyl)-28-((3-(1-(3,5-dichlorophenyl)-3-(3,3-dimethylmorpholine-4-carbonyl)-7-methoxy-1,4-dihydrochromeno|4,3-clpyrazol-8-yl)phenyl)amino)-7,13-bis(3-guanidinopropyl)-4-methyl-2,5,8,11,14,17,25,28-octaoxo-3,6,9,12,15,18,24-heptaazaoctacosyl)-1,4,7,10-tetraazacyclododecane-1,4,7-triyl)triacetic acid (Compound 84)

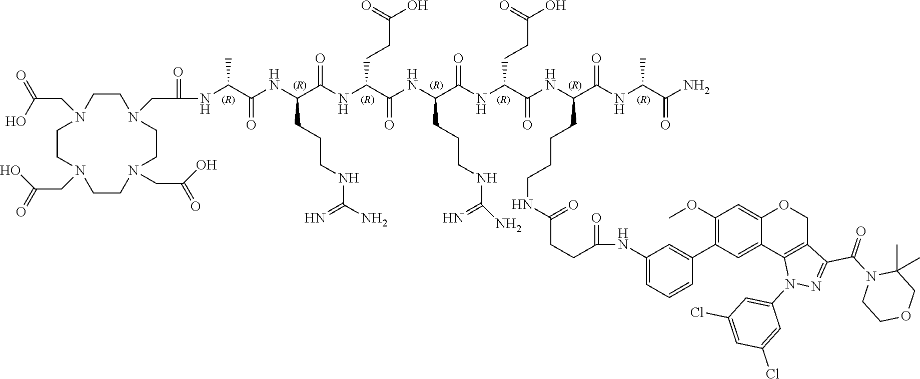

Compound 84 was prepared in a manner similar to that for Example 75. Calc'd for C&H₁₁₉C₁₂N₂₃O₂₄:1903.8, found [M+H]: 1904.8.

### Example 114:2,2′,2″-(10-((4R,7R,10R,13R,16R,19R)-19-(((R)-1-amino-1-oxopropan-2yl) carbamoyl)-7,13-bis(2-amino-2-oxoethyl)-28-((3-(1-(3,5-dichlorophenyl)-3-(3,3-dimethylmorpholine-4-carbonyl)-7-methoxy-1,4-dihydrochromeno|4,3-c]pyrazol-8-yl)phenyl)amino)-10,16-bis(hydroxymethyl)-4-methyl-2,5,8,11,14,17,25,28-octaoxo-3,6,9,12,15,18,24-heptaazaoctacosyl)-1,4,7,10-tetraazacyclododecane-1,4,7-triyl)triacetic acid (Compound 85)

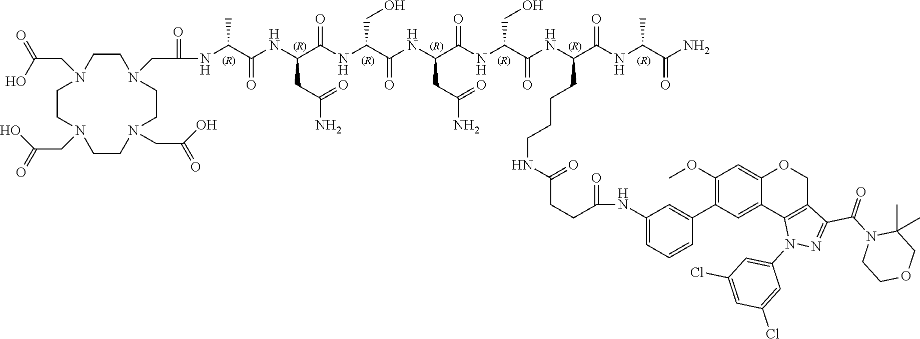

Compound 85 was prepared in a manner similar to that for Example 75. Calc'd for C₇₆H₁₀₃C₁₂N₁₉O₂₄:1735.7, found [M/2+H]: 869.8.

### Example 115:2,2′,2″-(10-((4R,7R,10R,13R,16R,19R,22R)-23-amino-10,13,16,19-tetrakis(2carboxyethyl)-7-(4-(4-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-4-oxobutanamido)butyl)-4,22-dimethyl-2,5,8,11,14,17,20,23-octaoxo-3,6,9,12,15,18,21-heptaazatricosyl)-1,4,7,10-tetraazacyclododecane-1,4,7-triyl)triacetic acid (Compound 87)

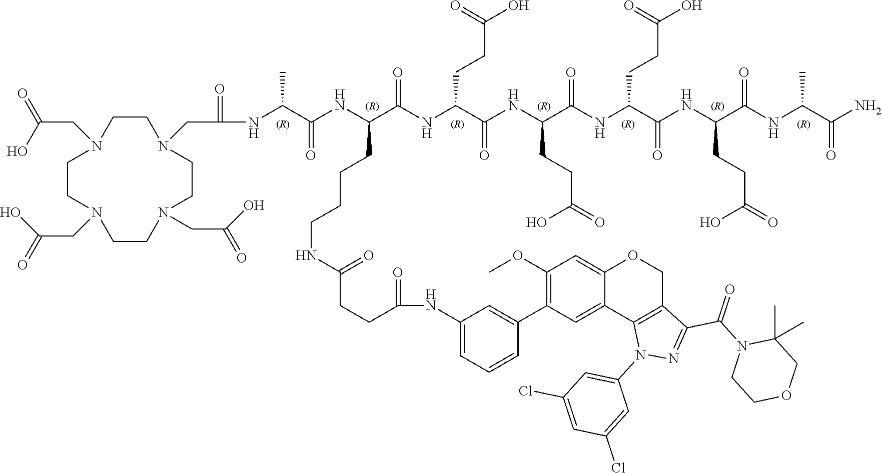

Compound 87 was prepared in a manner similar to that for Example 75. Calc'd for C₈₂H₁₀₉C₁₂N₁₇O₂₈:1849.7, found [M+H]: 1851.4.

### Example 116: (4R,7R,10R,13R,16R)-4-(((R)-1-amino-1-oxopropan-2-yl) carbamoyl)-7,13bis(2-carboxyethyl)-10-(4-(4-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-4-oxobutanamido)butyl)-6,9,12,15-tetraoxo-16-((R)-2-(2-(4,7,10-tris(carboxymethyl)-1,4,7,10-tetraazacyclododecan-1-yl)acetamido) propanamido)-5,8,11,14-tetraazanonadecanedioic acid (Compound 88)

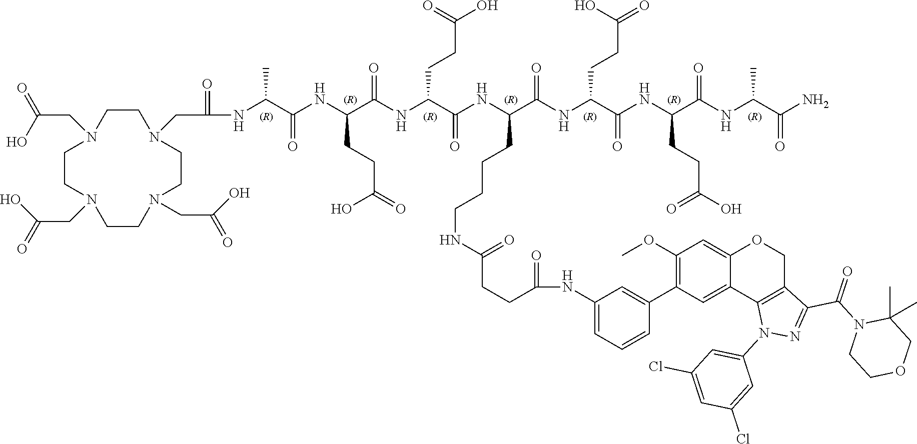

Compound 88 was prepared in a manner similar to that for Example 75. Calc'd for C₈₂H₁₀₉C₁₂N₁₇O₂₈:1849.7, found [M+H]: 1851.0.

### Example 117:2,2′,2″-(10-((6R,9R,12R,15R,18R,21R)-1-amino-6-(((R)-1-amino-1oxopropan-2-yl) carbamoyl)-15-(2-carboxyethyl)-12-(4-(4-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-4-oxobutanamido)butyl)-9,18-bis(3-guanidinopropyl)-1-imino-21-methyl-8,11,14,17,20,23-hexaoxo-2,7,10,13,16,19,22-heptaazatetracosan-24-yl)-1,4,7,10-tetraazacyclododecane-1,4,7-triyl)triacetic acid (Compound 89)

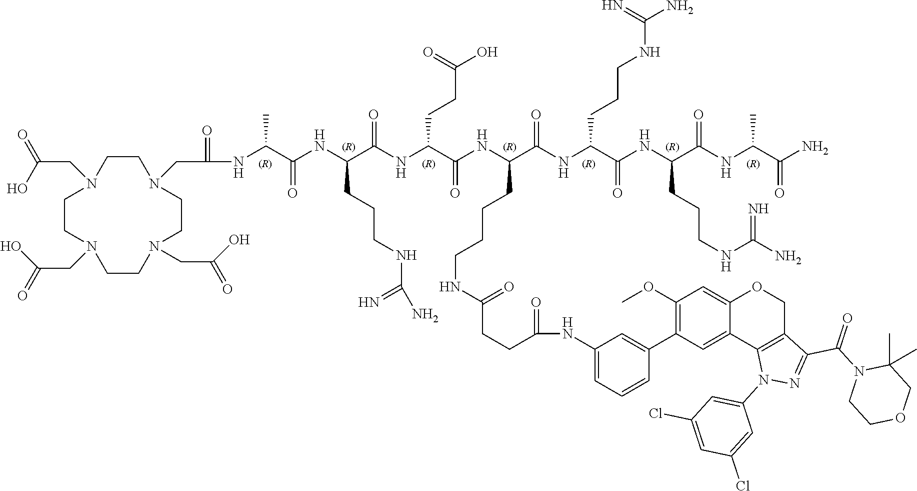

Compound 89 was prepared in a manner similar to that for Example 75. Calc'd for C₈₅H₁₂₄C₁₂N₂₆O₂₂:1930.9, found [M+H]: 1931.5.

### Example 118:2,2′,2″-(10-((4R,7R,10R,13R,16R,19R)-19-(((R)-1-amino-1-oxopropan-2yl) carbamoyl)-7,10,13,16-tetrakis(2-carboxyethyl)-28-((3-(1-(3,5-dichlorophenyl)-3-(3,3-dimethylmorpholine-4-carbonyl)-7-methoxy-1,4-dihydrochromeno|4,3-clpyrazol-8-yl)phenyl)amino)-4-methyl-2,5,8,11,14,17,25,28-octaoxo-3,6,9,12,15,18,24-heptaazaoctacosyl)-1,4,7,10-tetraazacyclododecane-1,4,7-triyl)triacetic acid (Compound 90)

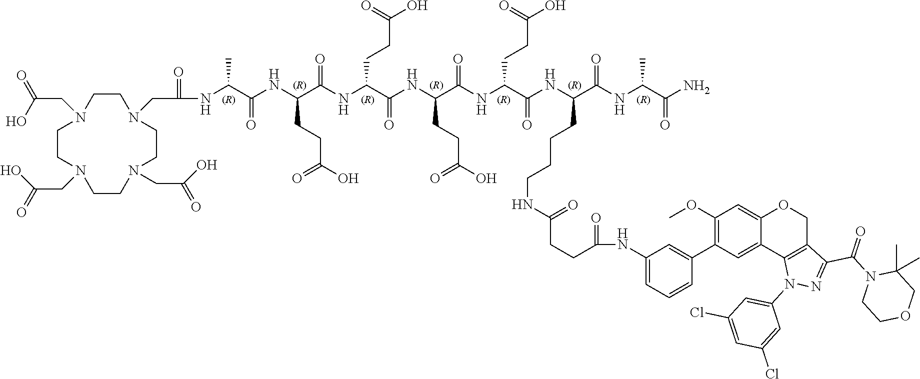

Compound 90 was prepared in a manner similar to that for Example 75. Calc'd for C₈₂H₁₀₉C₁₂N₁₇O₂₈:1849.7, found [M/2+2H]²⁺: 926.8.

### Example 119: (Compound 91)

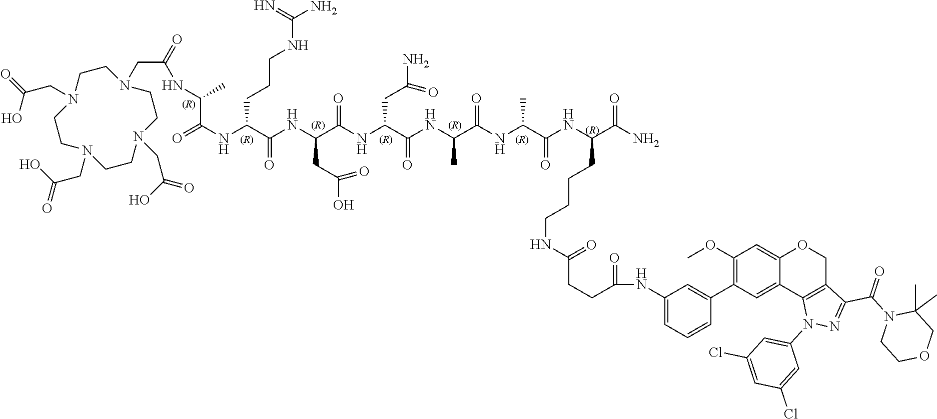

Compound 91 was prepared in a manner similar to that for Example 75. Calc'd for C₈₂H₁₀₉C₁₂N₁₇O₂₈:1789.8, found [M/2+2H]²⁺: 896.5.

### Example 120:2,2′,2″-(10-((8R,11R,14R,17R,20R,23R)-8-(((R)-1-amino-1-oxopropan-2yl) carbamoyl)-11,14,17,20-tetrakis(2-carboxyethyl)-28-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-23-methyl-2,10,13,16,19,22,25,28-octaoxo-3,9,12,15,18,21,24-heptaazaoctacosyl)-1,4,7,10-tetraazacyclododecane-1,4,7-triyl)triacetic acid (Compound 92)

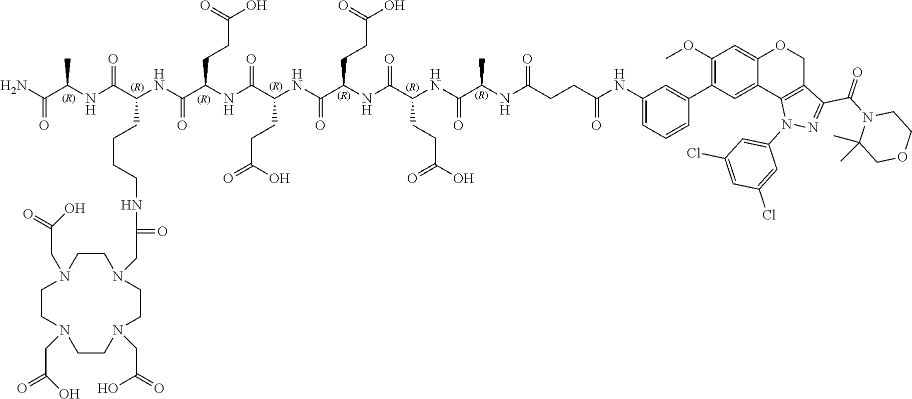

Compound 92 was prepared in a manner similar to that for Example 75. Calc'd for C₇₉H₁₀₉C₁₂N₂₁O23:1849.7, found [M/2+2H]+2:926.7.

### Example 121: (Compound 93)

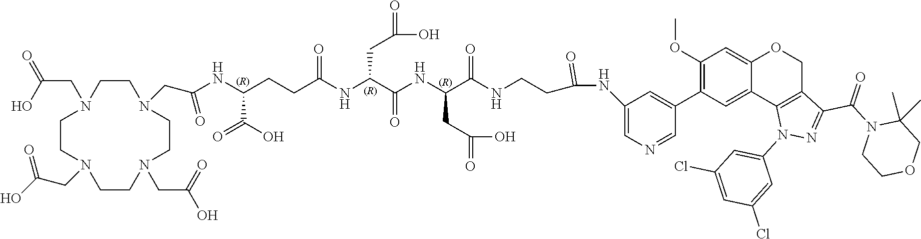

Compound 93 was prepared in a manner similar to that for Example 75. Calc'd for C₆₁H₇₅C₁₂N₁₃O₂₁:1395.5, found [M+H]⁺: 1396.6.

### Example 122: (Compound 94)

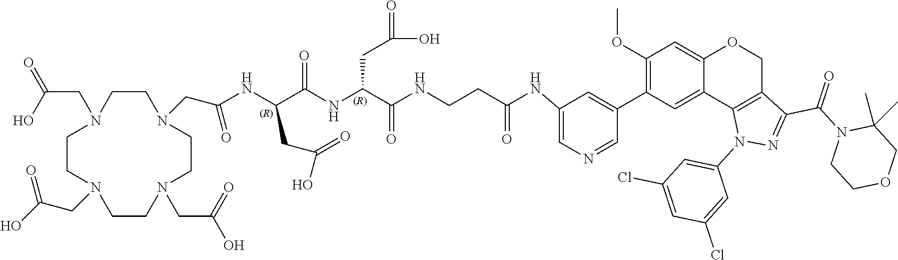

Step 1: Compound 94 was prepared in a manner similar to that for Example 75. Calc'd for C₅₆H₆₈C₁₂N₁₂O₁₈:1266.4, found [M+H]⁺: 1267.3.

### Example 123:2,2′,2″-(10-((4R,7R,10R,13R,16R,19R,22R)-13-(2-amino-2-oxoethyl)-22carbamoyl-10-(carboxymethyl)-31-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-7-(3-guanidinopropyl)-4,16,19-trimethyl-2,5,8,11,14,17,20,28,31-nonaoxo-3,6,9,12,15,18,21,27-octaazahentriacontyl)-1,4,7,10-tetraazacyclododecane-1,4,7-triyl)triacetic acid (Compound 95)

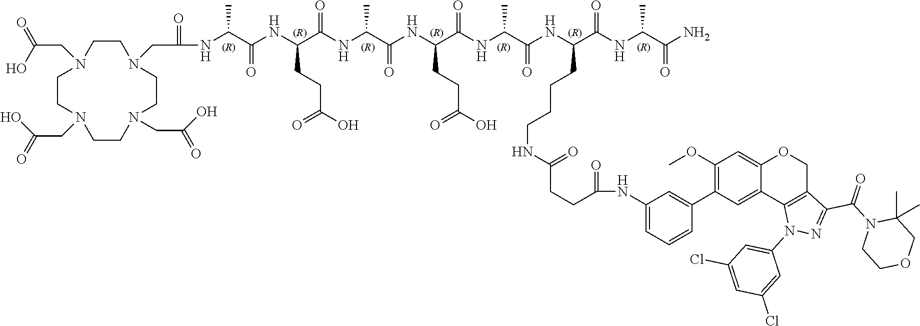

Compound 95 was prepared in a manner similar to that for Example 75. Calc'd for C₇₈H₁₀₅C₁₂N₁₇O₂₄:1733.7, found [M/2+H]: 868.7.

### Example 124:2,2′,2″-(10-((4R,7R,10R,13R,16R,19R,22R)-23-amino-13,19-bis(2carboxyethyl)-7-(4-(4-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-4-oxobutanamido)butyl)-4,10,16,22-tetramethyl-2,5,8,11,14,17,20,23-octaoxo-3,6,9,12,15,18,21-heptaazatricosy])-1,4,7,10-tetraazacyclododecane-1,4,7-triyl)triacetic acid (Compound 96)

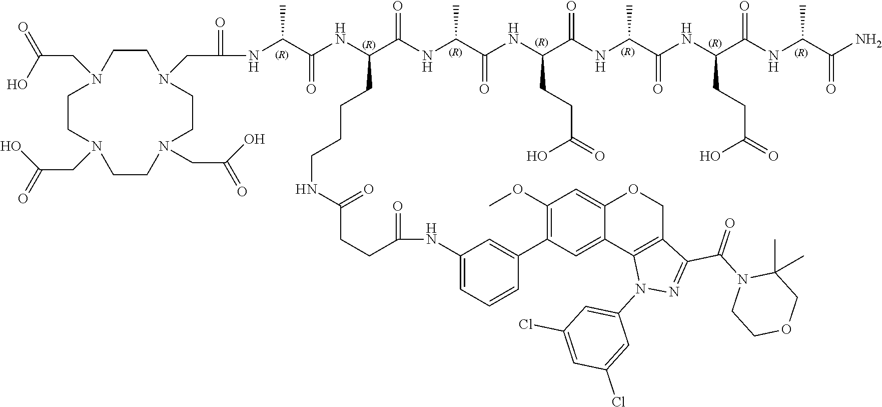

Compound 96 was prepared in a manner similar to that for Example 75. Calc'd for C₇₈H₁₀₅C₁₂N₁₇O₂₄:1733.7, found [M/3+3H]3+: 580.1.

### Example 125:2,2′,2″-(10-((4R,7R,10R,13R,16R,19R,22R)-23-amino-7,16-bis(2-amino-2oxoethyl)-13-(4-(4-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-4-oxobutanamido)butyl)-10,19-bis(hydroxymethyl)-4,22-dimethyl-2,5,8,11,14,17,20,23-octaoxo-3,6,9,12, 15,18,21-heptaazatricosyl)-1,4,7,10-tetraazacyclododecane-1,4,7-triyl)triacetic acid (Compound 97)

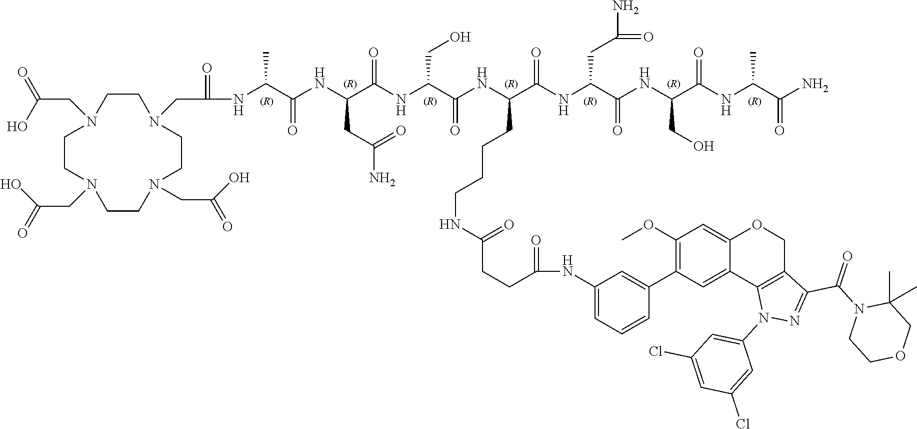

Compound 97 was prepared in a manner similar to that for Example 75. Calc'd for C₇₆H₁₀₃C₁₂N₁₉O₂₄:1735.7, found [M/2+2H]: 869.6.

### Example 126: (4R,7R,10R,13R,16R,19R,22R)-16-(4-aminobutyl)-7,10,13-tris(2carboxyethyl)-4-(((R)-1,6-diamino-1-oxohexan-2-yl) carbamoyl)-22-((R)-16-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-6,9,12-trimethyl-4,7,10,13,16-pentaoxo-2-(4-(2-(4,7,10-tris(carboxymethyl)-1,4,7,10-tetraazacyclododecan-1-yl)acetamido)butyl)-3,6,9,12-tetraazahexadecanamido)-19-(4-hydroxybenzyl)-6,9,12,15,18,21-hexaoxo-5,8,11,14,17,20-hexaazapentacosanedioic acid (Compound 98)

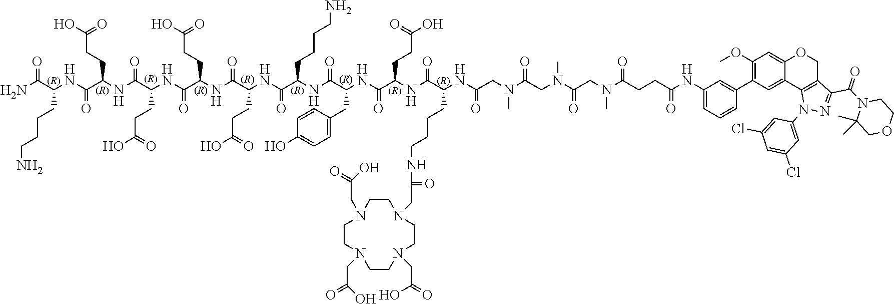

Compound 98 was prepared in a manner similar to that for Example 75. Calc'd for C₁₁₁H₁₅₄Cl₂N₂₄O₃₆:2469, found [M/3+H]⁺: 824.7.

### Example 127:2,2′,2″-(10-((7R,10R,13R)-7-amino-10,13-bis(carboxymethyl)-18-((5-(1-(3,5dichlorophenyl)-3-(3,3-dimethylmorpholine-4-carbonyl)-7-methoxy-1,4-dihydrochromeno|4,3-c|pyrazol-8-yl) pyridin-3-yl)amino)-2,8,11,14,18-pentaoxo-3,9,12,15-tetraazaoctadecyl)-1,4,7,10-tetraazacyclododecane-1,4,7-triyl)triacetic acid (Compound 99)

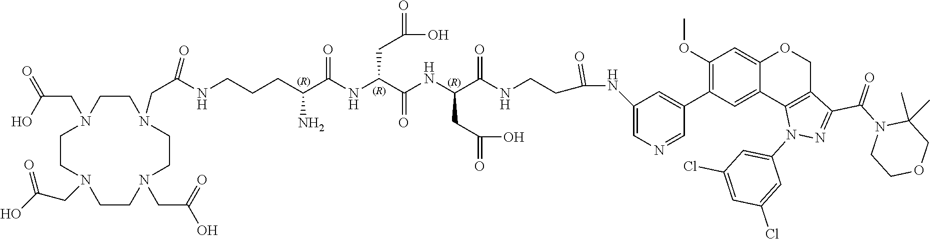

Compound 99 was prepared in a manner similar to that for Example 75. Calc'd for C₆₁H₇₈C₁₂N₁₄O₁₉:1380.5, found [M+H]: 1381.6.

### Example 128: (4R,7R,10R,13R,16R,19R,22R)-4-(((6R,9R,12R)-1-amino-6-(((R)-1-amino-1oxopropan-2-yl) carbamoyl)-21-((3-(1-(3,5-dichlorophenyl)-3-(3,3-dimethylmorpholine-4-carbonyl)-7-methoxy-1,4-dihydrochromeno|4,3-c|pyrazol-8-yl)phenyl)amino)-9-(3-guanidinopropyl)-1-imino-8,11,18,21-tetraoxo-2,7,10,17-tetraazahenicosan-12-yl) carbamoyl)-19-(4-(4-((3-(1-(3,5-dichlorophenyl)-3-(3,3-dimethylmorpholine-4-carbonyl)-7-methoxy-1,4-dihydrochromeno 4,3-c]pyrazol-8-yl)phenyl)amino)-4-oxobutanamido)butyl)-22-((R)-5-guanidino-2-((R)-2-(2-(4,7,10-tris(carboxymethyl)-1,4,7,10-tetraazacyclododecan-1-yl)acetamido) propanamido) pentanamido)-7,13,16-tris(3-guanidinopropyl)-10-methyl-6,9,12,15,18,21-hexaoxo-5,8,11,14,17,20-hexaazapentacosanedioic acid (Compound 100)

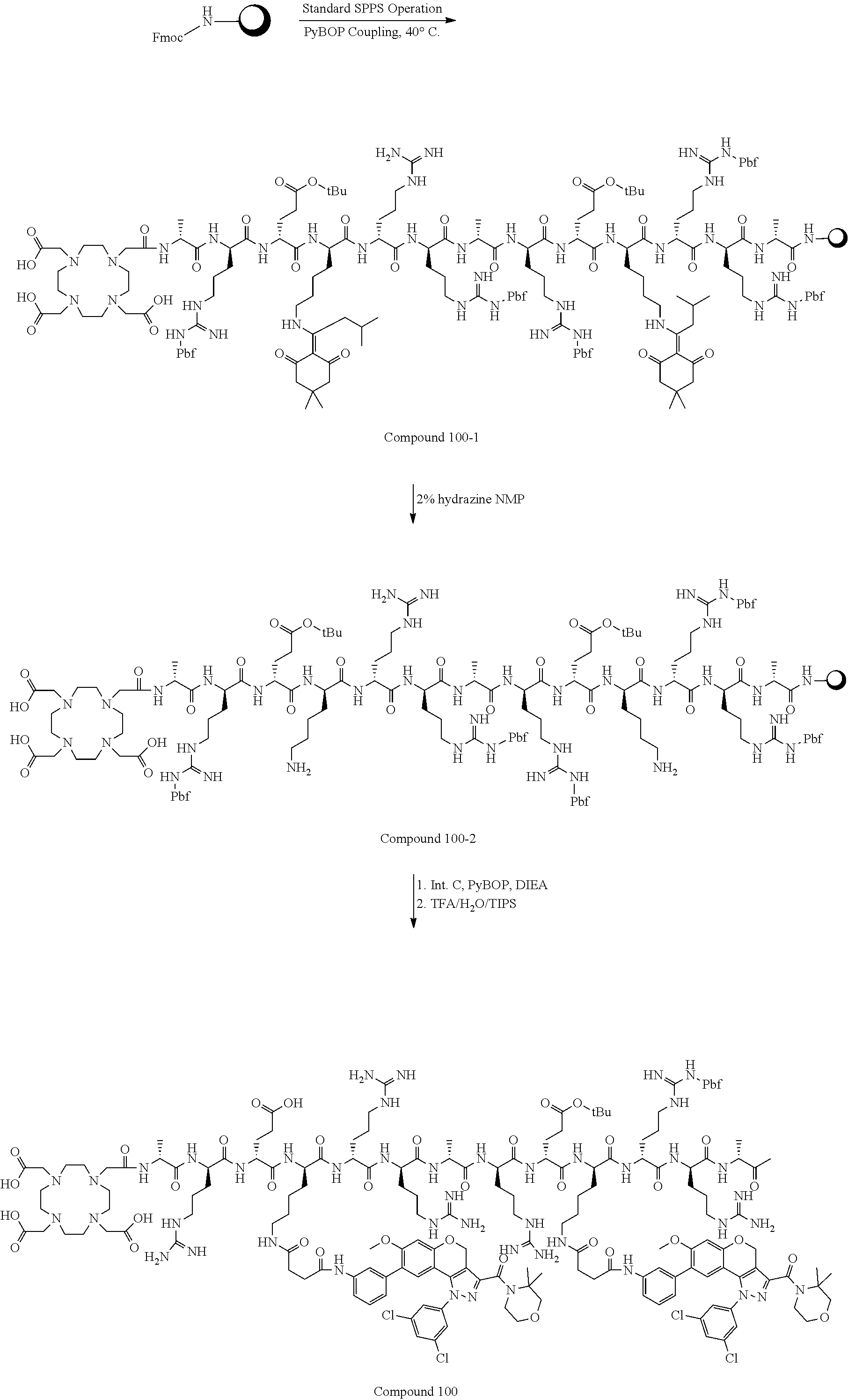

Step 1. Synthesis of Compound 100-1: The automated SPPS was executed with a Syro II peptide synthesizer (Biotage). Resin pre-swelling: The Rink amide resin (100 mg/tube, 100-200 mesh, 0.62 mmol/g, total 1 tube)_{w} as swelled in NMP (1 mL/tube) for 15 min at room temperature under nitrogen atmosphere. The resin was then washed 4 times with NMP (1 mL/tube). Parameter: incubation time: 15 min, vortex time: 15 s, break time: 1 min, temperature: room temperature, empty time: 1 min. SPPS coupling cycle: A) Fmoc de-protection: the resin (100 mg/tube, total 1 tube)_{w} as treated with 20% of piperidine in NMP (1 mL) for 20 min at room temperature under nitrogen atmosphere. The resin was then washed 4 times with NMP (1 mL/tube). Parameter: reaction time: 20 min, vortex time: 15 s, break time: 1 min, temperature: room temperature, empty time: 1 min. B) HATU coupling: the resin (100 mg/tube, total 1 tube) was treated with the mixture of amino acid or DOTA-NHS (4 eq.), HATU (4 eq.) and DIEA (8 eq.) in NMP (1 mL) for 45 min at 40° C. under a nitrogen atmosphere. Then, the resin was washed 4 times with NMP (1 mL/tube). Parameter: reaction time: 45 min, vortex time: 15 s, break time: 3 min, temperature: 40° C., empty time: 1 min.

Step 2: Synthesis of Compound 100-2: Compound 100-1 on resin (100 mg)_{w} as swelled with NMP (5 mL) for 15 min at room temperature. The solvent was then removed. To the resin (100 mg) in 25 mL reactor was added 2% hydrazine in NMP (5 mL) at room temperature under nitrogen atmosphere. The mixture was bubbled for 2 h and filtered, washed with NMP (5 mL×8). The reaction was monitored by LCMS.

Step 3: To the resin (100 mg) in a 25 mL reactor was added intermediate C (4 eq.), HATU (2 eq.) and DIEA (4 eq.) in NMP (5 mL). After the reaction mixture was incubated for 2 h at 40° C., the solvent was removed and the resin was washed with NMP (5 mL×6).

Step 4: The resin was treated with the cleavage solution (v v v v TFA/H₂O/TIS/DODT=37:1:1:1) for 2 h at room temperature. After filtration, the filtrate was concentrated under reduced pressure. The residue was precipitated with cold ether. After centrifugation, the solid was collected and purified by prep-HPLC. The desired fractions were combined and lyophilized to afford Compound 100 as a white solid (7.2 mg, 2.91%, 85.1% purity). Calc'd for C₆₁H₇₈C₁₂N₁₄O₁₉:3387.5, found [M+4H]++: 848.8.

### Example 129: Indium Complex of Compound 54 (Compound 54A)

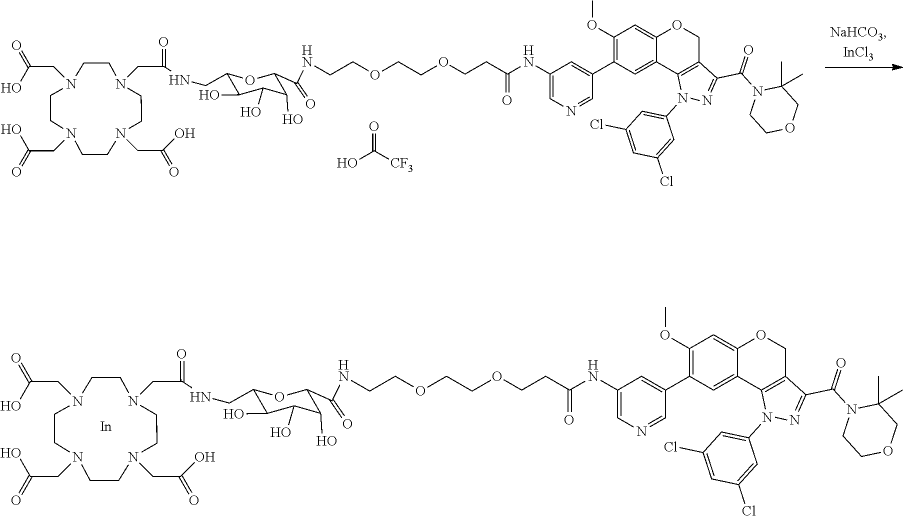

Into a 8 mL flask was added a mixture of 2,2′,2″-(10-(2-((((2S,3R,4R,5R,6S)-6-((2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid (25 mg, 1 Eq, 19 μmol), sodium bicarbonate (10 mg, 4.6 μL, 6.3 Eq, 0.12 mmol), indium trichloride (15 mg, 4.3 uL, 3.6 Eq, 68 μmol), ACN (0.2 mL), and water (0.1 mL). The mixture was stirred for 1 hour at 80° C. The mixture was diluted with DMSO(4 mL), filtered and the filtrate was purified by Prep-HPLC to provide indium (III) 2,2′,2″-(10-(2-((((2S,3R,4R,5R,6S)-6-((2-(2-(3-((5-(1-(3,5-dichlorophenyl)-3-(3,3-dimethylmorpholine-4-carbonyl)-7-methoxy-1,4-dihydrochromeno[4,3-c]pyrazol-8-yl) pyridin-3-yl)amino)-3-oxopropoxy)ethoxy)ethyl) carbamoyl)-3,4,5-trihydroxytetrahydro-2H-pyran-2-yl)methyl)amino)-2-oxoethyl)-1,4,7,10-tetraazacyclododecane-1,4,7-triyl)triacetate (18.3 mg, 12.8 μmol, 67%) as a white solid. Calc'd for C₅₉H₇₄C₁₂InN10013:1425. 4, found [M+H]: 1426.3, 1428.3.

### Example 130: Indium Complex of Compound 89 (Compound 89A)

2,2′,2″-(10-((6R,9R,12R, 15R, 18R,21R)-1-amino-6-(((R)-1-amino-1-oxopropan-2-yl) carbamoyl)-15-(2-carboxyethyl)-12-(4-(4-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-4-oxobutanamido)butyl)-9,18-bis(3-guanidinopropyl)-1-imino-21-methyl-8,11,14,17,20,23-hexaoxo-2,7,10, 13, 16, 19,22-heptaazatetracosan-24-yl)-1,4,7,10-tetraazacyclododecane-1,4,7-triyl)triacetic acid was treated with sodium bicarbonate and indium trichloride in a manner similar to Example 129 to provide Compound 89A. Calc'd for C₈₅H₁₂₁C₁₂InN26O22:2042.8, found [M/2+2H]: 1023.5.

### Example 131: Indium Complex of Compound 92 (Compound 92A)

2,2′,2″-(10-((8R, 11R, 14R, 17R,20R,23R)-8-(((R)-1-amino-1-oxopropan-2-yl) carbamoyl)-11,14,17,20-tetrakis(2-carboxyethyl)-28-((3-(1-(3,5-dichlorophenyl)-3-(3,3-dimethylmorpholine-4-carbonyl)-7-methoxy-1,4-dihydrochromeno[4,3-c]pyrazol-8-yl)phenyl)amino)-23-methyl-2,10, 13, 16, 19,22,25,28-octaoxo-3,9,12, 15, 18,21,24-heptaazaoctacosyl)-1,4,7,10-tetraazacyclododecane-1,4,7-triyl)triacetic acidwas treated with sodium bicarbonate and indium trichloride in a manner similar to Example 129 to provide Compound 89A. Calc'd for C₈₂H₁₀₆C₁₂InN17028:1961.6, found [M/2+2H]: 982.7.

### Example 132: Parenteral Pharmaceutical Composition

To prepare a parenteral pharmaceutical composition suitable for administration by injection (subcutaneous, intravenous), 0.001-500 mg of a compound Formula (I) Formula (B), or Formula (A), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Biology Examples

Measurement of agonist cAMP production by activation of human FSHR

Two days prior to the assay, 5,000 CHO—K₁ human FSHR cells (Eurofins #95-0119C₂) stably expressing the functional human follicle-stimulating hormone receptor were plated in a 96-well tissue culture-treated plate in growth media supplemented with the appropriate selection agent. Cells were cultured at 37° C., 5% CO₂ and 95% humidity. On the day of the assay, the growth media was discarded and the cells were treated with test compound or FSH (Eurofins #92-1166) prepared in assay buffer [1x Hank's Balanced Salt Solution (ThermoFisher #SH3058802), 0.5 mM HEPES (Biopioneer Cat #C₀₁₁₃) pH 7.4, 0.1% bovine serum albumin (Fisher Scientific Cat #BP1600), 1 mM 3-Isobutyl-1-methylxanthine (IBMX, VWR #200002-790)] to generate dose response curves. The cells were incubated for 30 minutes at 37° C. (the final concentrations of the compound ranged from 0-10,000 nM). After the 30-minute incubation at 37° C., lysis buffer (HRTF CAMP kit, Cisbio #62AM5PEB)_{w} as added directly to the cells and incubated for 30-minutes at room temperature. The diluted lysate was transferred and CAMP detection and visualization antibodies (HRTF CAMP kit, Cisbio #62AM5PEB) were added and incubated for 1-24 hours at room temperature. The time-resolved fluorescent signal was read with a Tecan M1000Pro (Tecan) multiplate reader. The intracellular cAMP concentrations were calculated by regression to a standard curve and were plotted vs. the concentration of the agonists. The concentration of test compound or FSH required to generate a half maximal response EC50 were calculated using standard curve-fitting methods. All data manipulations were performed using GraphPad Prism v9.3.1 (GraphPad, San Diego, CA).

TABLE A
Representative Functional Activity

| Cmpd No. | Compound Structure/Description | hFSHR Agonist cAMP EC₅₀* |
|---|---|---|
| 1 | Compound 1 | A |
| 1A | In complex of Compound 1 | A |
| 2 | Compound 2 | A |
| 2A | In Complex of Compound 2 | A |
| 3 | Compound 3 | A |
| 3A | In Complex of Compound 3 | A |
| 4 | Compound 4 | A |
| 5 | Compound 5 | B |
| 5A | In Complex of Compound 5 | A |
| 6 | Compound 6 | A |
| 6A | In Complex of Compound 6 | A |
| 7 | Compound 7 | A |
| 8 | Compound 8 | B |
| 8A | In complex of Compound 8 | A |
| 9 | Compound 9 | B |
| 10 | Compound 10 | A |
| 10A | In complex of Compound 10 | A |
| 11 | Compound 11 | A |
| 11A | In complex of Compound 11 | A |
| 12 | Compound 12 | B |
| 13 | Compound 13 | B |
| 14 | Compound 14 | A |
| 14A | In complex of Compound 14 | A |
| 15 | Compound 15 | A |
| 15A | In complex of Compound 15 | A |
| 16 | Compound 16 | B |
| 16A | In complex of Compound 16 | A |
| 17 | Compound 17 | A |
| 17A | In complex of Compound 17 | B |
| 17B | Lu complex of Compound 17 | A |
| 18 | Compound 18 | B |
| 18A | In complex of Compound 18 | A |
| 19 | Compound 19 | A |
| 19A | In complex of Compound 19 | A |
| 20 | Compound 20 | A |
| 20A | In complex of Compound 20 | A |
| 21 | Compound 21 | A |
| 21A | In complex of Compound 21 | A |
| 22 | Compound 22 | A |
| 22A | In complex of Compound 22 | A |
| 23 | Compound 23 | A |
| 23A | In complex of Compound 23 | B |
| 24 | Compound 24 | B |
| 24A | In complex of Compound 24 | A |
| 25 | Compound 25 | C |
| 26 | Compound 26 | A |
| 26A | In complex of Compound 26 | A |
| 27 | Compound 27 | A |
| 28 | Compound 28 | A |
| 29 | Compound 29 | C |
| 30 | Compound 30 | B |
| 31 | Compound 31 | B |
| 32 | Compound 32 | B |
| 32A | In complex of Compound 32 | A |
| 33 | Compound 33 | C |
| 34 | Compound 34 | B |
| 35 | Compound 35 | C |
| 36 | Compound 36 | A |
| 37 | Compound 37 | B |
| 38 | Compound 38 | B |
| 39 | Compound 39 | B |
| 40 | Compound 40 | B |
| 41 | Compound 41 | B |
| 42 | Compound 42 | B |
| 43 | Compound 43 | B |
| 44 | Compound 44 | A |
| 44A | In complex of Compound 44 | A |
| 45 | Compound 45 | A |
| 46 | Compound 46 | B |
| 47 | Compound 47 | B |
| 49 | Compound 49 | C |
| 50 | Compound 50 | C |
| 51 | Compound 51 | A |
| 52 | Compound 52 | A |
| 53 | Compound 53 | A |
| 54 | Compound 54 | A |
| 54A | In complex of Compound 54 | B |
| 55 | Compound 55 | B |
| 56 | Compound 56 | B |
| 57 | Compound 57 | B |
| 58 | Compound 58 | B |
| 59 | Compound 58 | C |
| 60 | Compound 60 | B |
| 61 | Compound 61 | B |
| 62 | Compound 62 | B |
| 63 | Compound 63 | B |
| 64 | Compound 64 | A |
| 65 | Compound 65 | B |
| 66 | Compound 66 | B |
| 67 | Compound 67 | A |
| 68 | Compound 68 | A |
| 69 | Compound 69 | A |
| 70 | Compound 70 | A |
| 71 | Compound 71 | A |
| 72 | Compound 72 | B |
| 73 | Compound 73 | A |
| 74 | Compound 74 | B |
| 75 | Compound 75 | B |
| 76 | Compound 76 | B |
| 77 | Compound 77 | B |
| 78 | Compound 78 | A |
| 79 | Compound 79 | A |
| 80 | Compound 80 | B |
| 81 | Compound 81 | A |
| 82 | Compound 82 | A |
| 83 | Compound 83 | B |
| 84 | Compound 84 | B |
| 85 | Compound 85 | B |
| 86 | Compound 86 | B |
| 87 | Compound 87 | B |
| 88 | Compound 88 | B |
| 89 | Compound 89 | B |
| 89A | In complex of Compound 89 | B |
| 90 | Compound 90 | B |
| 91 | Compound 91 | B |
| 92 | Compound 92 | B |
| 92A | In complex of Compound 92 | B |
| 93 | Compound 93 | B |
| 94 | Compound 94 | B |
| 95 | Compound 95 | B |
| 97 | Compound 97 | B |
| 98 | Compound 98 | B |
| 99 | Compound 99 | B |
| 100 | Compound 100 | C |

*A is <10 nM; B is 10-100 nM; C is >100 nM

Other embodiments are set forth in the following claims, along with the full scope of equivalents to which such claims are entitled.

What is claimed is:

1. A compound of Formula (A), or a pharmaceutically acceptable salt thereof, 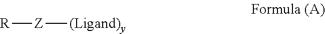
wherein:
R is L^{A}-L^{B}-R⁶, -L^{A}-(L^{B}-R⁶)₂, or -L^{A}-(L^{B}-R⁶) 3;
L^{A} is a linker or is absent;
L^{B} is a linker or is absent;
R⁶ is a chelating moiety or a radionuclide complex thereof;
Z is C₁-C₆ alkylene, C₁-C₆ alkylene-O—, —O—C₁-C₆ alkylene-, —C(═O)NR¹⁰—, —NR¹⁰C(═O)—, —NR¹⁰—, —O—, —S—, —S(═O)—, —SO₂—, —NR¹⁰C, (═O)NR¹⁰—, or —CR¹⁰—NO—;
each R¹⁰ is independently H or unsubstituted C₁-C₄ alkyl;
Ligand is a small molecule modulator of the follicle-stimulating hormone receptor (FSHR); and
y is 1, 2 or 3.

2. The compound of claim 1, or a pharmaceutically acceptable salt thereof, wherein R is L^{B}-R⁶.

3. The compound of claim 1 or 2, or a pharmaceutically acceptable salt thereof, wherein Ligand is a small molecule agonist of FSHR.

4. The compound of claim 1 or 2, or a pharmaceutically acceptable salt thereof, wherein Ligand is a small molecule antagonist of FSHR.

5. The compound of any one of claims 1-4, or a pharmaceutically acceptable salt thereof, wherein Ligand comprises a thiazolidinone (TZD), a diketopiperazine, a hexahydroquinoline, a thienopyrimidine, a piperidine carboxyamide, an acyltryptophanol, a pyrrolobenzodiazepine, an aminoalkylamide, an isoxazolyl-thiazolyl, a dihydropyrrolo[2,1-a]isoquinoline, a dihydroimidazo[5,1-A]isoquinoline, a dihydrobenzoindazole, a fused tricylic imidazole, a fused tricylic pyrazole, a 1,4-dihydrochromeno[4,3-c]pyrazole, or a dihydro-1H-benzo[g]indole.

6. The compound of any one of claims 1-4, or a pharmaceutically acceptable salt thereof, wherein Ligand comprises a dihydropyrrolo[2,1-a]isoquinoline, a dihydroimidazo[5,1-A]isoquinoline, a dihydrobenzoindazole, a 1,4-dihydrochromeno[4,3-c]pyrazole, or a dihydro-1H-benzo[g]indole.

7. The compound of any one of claims 1-4, or a pharmaceutically acceptable salt thereof, wherein Ligand comprises a naphthalene sulfonic acid, a (bis)sulfonic acid, a (bis)benzamide, a tetrahydroquinoline (THQ), a benzamide, a naphthalene sulfonic acid, or a tetrahydroquinoline.

8. The compound of claim 5, wherein the fused tricylic imidazole is a dihydrobenzo-imidazole.

9. The compound of claim 5, wherein the fused tricylic imidazole is a dihydrobenzo-pyrazole.

10. The compound of claim 1, wherein the compound of Formula (A) has the structure of Formula (B), or a pharmaceutically acceptable salt thereof: 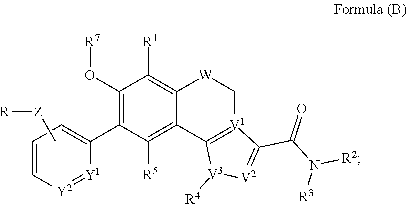
wherein:
R¹ is H, F, Cl, Br or I;
W is O or —CH₂—:
V¹ is C or N;
V² is C, CR⁸ or N;
V³ is CR⁸ or N;
R² and R³ are each independently substituted or unsubstituted alkyl;
or R² and R³ with the nitrogen to which they are connected form a substituted or unsubstituted 5 to 8-membered heterocycloalkyl;
R² is a substituted or unsubstituted aryl or substituted or unsubstituted heteroaryl;
R⁵ is H, F, Cl, Br or I;
Y¹ and Y² are each independently CR⁹ or N;
Z is C₁-C₆ alkylene, —C(═O)NR¹⁰—, —NR¹⁰C(═O)—, —NR¹⁰—, —O—, —S—, —S(═O)—, —SO₂—, —NR¹⁰C(═O)NR¹⁰—, or —CR¹⁰—NO—;
R is -L¹-R⁶, —CH-(L¹-R⁶)₂, substituted or unsubstituted C₁-C₁₀alkylene-CH-(L¹-R⁶)₂, substituted or unsubstituted 2 to 10 membered heteroalkylene-CH-(L¹-R⁶)₂, —N-(L¹-R⁶)₂, substituted or unsubstituted C₁-C₁₀alkylene-N(L¹-R⁶)₂, substituted or unsubstituted 2 to 10 membered heteroalkylene-N(L¹-R⁶)₂, or —(CH₂CH₂O)_{q}—CH₂CH₂N(L¹-R⁶) 2;
q is 1, 2, 3, 4, 5 or 6;
L¹ is a linker or is absent;
R⁶ is a chelating moiety or a radionuclide complex thereof;
R⁷ is H or substituted or unsubstituted alkyl;
each R⁸ is independently H, —N(R¹¹)₂, F, Cl, Br, I, or —OR¹¹;
each R⁹ is independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —N(R¹²)₂, or —OR¹²;
each R¹⁰ is independently H or unsubstituted C₁-C₄ alkyl;
each R¹¹ is independently H or unsubstituted C₁-C₄ alkyl; and
each R¹² is independently H or unsubstituted C₁-C₄ alkyl.

11. The compound of claim 1 or claim 10, wherein the compound has the structure of Formula (I), or a pharmaceutically acceptable salt thereof: 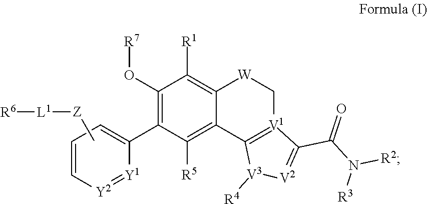
wherein:
R¹ is H, F, Cl, Br or I;
W is O or CH₂;
V¹ is C or N;
V² is C, CR⁸ or N;
V³ is CR⁸ or N;
R² and R³ are each independently substituted or unsubstituted alkyl;
or R² and R³ with the nitrogen to which they are connected form a substituted or unsubstituted 5 to 8-membered heterocycloalkyl;
R⁴ is a substituted or unsubstituted aryl or substituted or unsubstituted heteroaryl;
R⁵ is H, F, Cl, Br or I;
Y¹ and Y² are each independently CR⁹ or N;
Z is C₁-C₆ alkylene, —C(═O)NR¹⁰—, —NR¹⁰C(═O)—, —NR¹⁰—, —O—, —S—, —S(═O)—, —SO₂—, —NR¹⁰C(—O)NR¹⁰—, or —CR¹⁰—NO—;
L¹ is a linker or is absent;
R⁶ is a chelating moiety or a radionuclide complex thereof;
R⁷ is H or substituted or unsubstituted alkyl;
each R⁸ is independently H, —N(R¹¹)₂, F, Cl, Br, I, —OR¹¹;
each R⁹ is H, halogen, substituted or unsubstituted C₁-C₄ alkyl, substituted or unsubstituted C₁-C₄ fluoroalkyl, substituted or unsubstituted 2 to 6 membered heteroalkyl, —CN, —N(R¹²)₂, or —OR¹²;
each R¹⁰ is independently H or unsubstituted C₁-C₄ alkyl;
each R¹¹ is independently H or unsubstituted C₁-C₄ alkyl; and
each R¹² is independently H or unsubstituted C₁-C₄ alkyl.

12. The compound of claim 11, or a pharmaceutically acceptable salt thereof, wherein the compound of Formula (I) has the structure of Formula (Ia), or a pharmaceutically acceptable salt thereof:

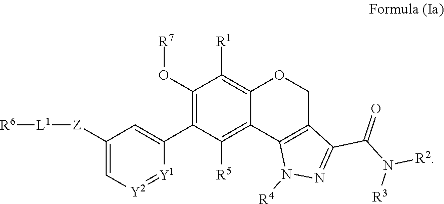

13. The compound of claim 11, or a pharmaceutically acceptable salt thereof, wherein the compound of Formula (I) has the structure of Formula (Ib), or a pharmaceutically acceptable salt thereof:

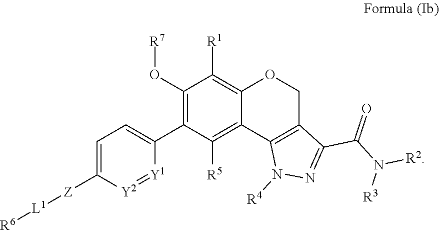

14. The compound of any one of claims 10-13, or a pharmaceutically acceptable salt thereof, wherein R¹ is H, F or Cl.

15. The compound of any one of claims 10-14, or a pharmaceutically acceptable salt thereof, wherein R⁵ is H or F.

16. The compound of any one of claims 10-15, or a pharmaceutically acceptable salt thereof, wherein each R⁹ is independently H, halogen, —CH₃, or —CF₃.

17. The compound of any one of claims 10-15, or a pharmaceutically acceptable salt thereof, wherein Y¹ and Y² are CH.

18. The compound of any one of claims 10-15, or a pharmaceutically acceptable salt thereof, wherein Y¹ is N and Y² is CH.

19. The compound of any one of claims 10-15, or a pharmaceutically acceptable salt thereof, wherein Y¹ is CH and Y² is N.

20. The compound of any one of claims 10-15, or a pharmaceutically acceptable salt thereof, wherein Y¹ is N and Y² is N.

21. The compound of any one of claims 10-20, or a pharmaceutically acceptable salt thereof, wherein R₇ is —CH₃.

22. The compound of any one of claims 10-21, or a pharmaceutically acceptable salt thereof, wherein R¹⁰ is H.

23. The compound of any one of claims 10-21, or a pharmaceutically acceptable salt thereof, wherein R¹⁰ is —CH₃.

24. The compound of claim 1, 10, or 11, or a pharmaceutically acceptable salt thereof, wherein the compound has one of the following structures, or a pharmaceutically acceptable salt thereof:

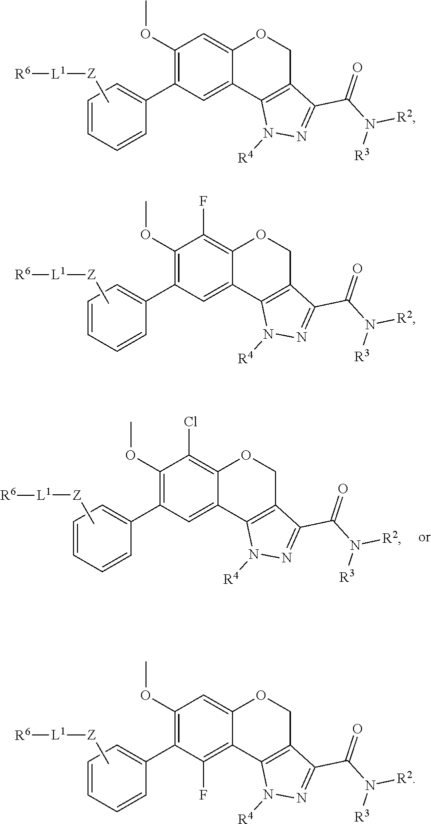

25. The compound of claim 1, 10, or 11, or a pharmaceutically acceptable salt thereof, wherein the compound has one of the following structures, or a pharmaceutically acceptable salt thereof:

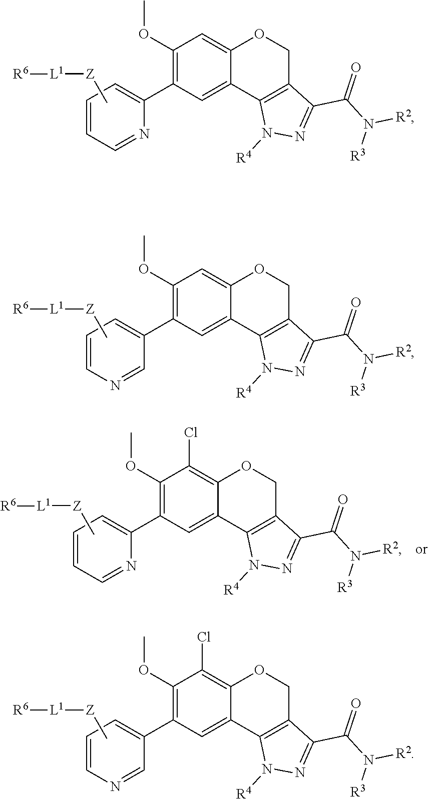

26. The compound of any one of claims 10-25, or a pharmaceutically acceptable salt thereof, wherein R² is —CH₃ and R³ is t-butyl.

27. The compound of any one of claims 10-25, or a pharmaceutically acceptable salt thereof, wherein R² and R³ with the nitrogen to which they are connected form: 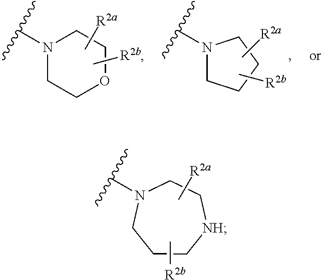
wherein R^{2a} and R^{2b} are each independently H, halogen, substituted or unsubstituted C₁-C₄ alkyl, substituted or unsubstituted C₁-C₄ fluoroalkyl, substituted or unsubstituted 2 to 6 membered heteroalkyl, —C(—O)R^{2c}, —CN, —NH₂, or —OH; wherein R^{2c} is substituted or unsubstituted C₁-C₄ alkyl or substituted or unsubstituted C₃-C₆ cycloalkyl.

28. The compound of claim 25, or a pharmaceutically acceptable salt thereof, wherein R^{2a} and R^{2b} are each —CH₃.

29. The compound of any one of claims 10-25, or a pharmaceutically acceptable salt thereof, wherein R² and R³ with the nitrogen to which they are connected form:

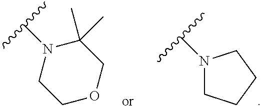

30. The compound of any one of claims 10-29, or a pharmaceutically acceptable salt thereof, wherein R⁴ is a substituted or unsubstituted phenyl or a substituted or unsubstituted 5 to 6-membered heteroaryl.

31. The compound of any one of claims 10-29, or a pharmaceutically acceptable salt thereof, wherein R⁴ is a substituted or unsubstituted pyridinyl, substituted or unsubstituted imidazolyl, substituted or unsubstituted pyrimidinyl, substituted or unsubstituted pyrazolyl, substituted or unsubstituted triazolyl, substituted or unsubstituted pyrazinyl, substituted or unsubstituted tetrazolyl, substituted or unsubstituted furyl, substituted or unsubstituted thienyl, substituted or unsubstituted isoxazolyl, substituted or unsubstituted thiazolyl, substituted or unsubstituted oxazolyl, substituted or unsubstituted isothiazolyl, substituted or unsubstituted pyrrolyl, substituted or unsubstituted pyridazinyl, substituted or unsubstituted triazinyl, substituted or unsubstituted oxadiazolyl, substituted or unsubstituted thiadiazolyl, and substituted or unsubstituted furazanyl.

32. The compound of any one of claims 10-29, or a pharmaceutically acceptable salt thereof, wherein R⁴ is 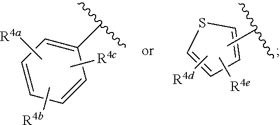
wherein R^{4a}, R^{4b}, R^{4c}, R^{4d} and R^{4e} are each independently H, halogen, substituted or unsubstituted C₁-C₄ alkyl, substituted or unsubstituted C₁-C₄ fluoroalkyl, substituted or unsubstituted 2 to 6 membered heteroalkyl, —CN, —NH₂, or —OH.

33. The compound of claim 32, or a pharmaceutically acceptable salt thereof, wherein R^{4a} and R^{4b} are each independently F, Cl, Br, I or —CH₃ and R⁴ is H.

34. The compound of claim 32, or a pharmaceutically acceptable salt thereof, wherein R^{4d} and R^{4e} are H.

35. The compound of any one of claims 1-29, or a pharmaceutically acceptable salt thereof, wherein R⁴ is

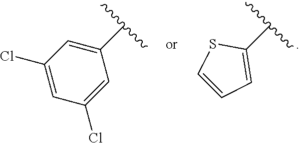 [SMILES: *c1cc(Cl)cc(Cl)c1 ; *c1cccs1]

36. The compound of any one of claims 1-35, or a pharmaceutically acceptable salt thereof, wherein Z is —C(═O)NH—, —NHC(═O)—, —O—, or —NHC(═O)NH—.

37. The compound of any one of claims 1-35, or a pharmaceutically acceptable salt thereof, wherein Z is —NHC(═O)— or —NHC(═O)NH—.

38. The compound of any one of claims 10-37, or a pharmaceutically acceptable salt thereof, wherein each R⁸ is H.

39. The compound of any one of claims 1-38, or a pharmaceutically acceptable salt thereof, wherein R⁶ is a chelating moiety selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6″-(((pyridine-2,6-diylbis(methylene))-bis(azanetriyl))tetrakis(methylene))-tetrapicolinic acid (H₄py₄pa); H₄py4pa-benzyl; 2,2′,2″-(1,4,7-triazacyclononane-1,4,7-triyl)triacetic acid (NOTA); 6,6′-((1,4,10,13-tetraoxa-7, 16-diazacyclooctadecane-7,16-diyl)bis(methylene))dipicolinic acid (macropa); 2,2′,2″,2″-(1,10-dioxa-4,7, 13, 16-tetraazacyclooctadecane-4,7, 13, 16-tetrayl)tetraacetic acid (crown); 6,6′-((ethane-1,2-diylbis((carboxymethyl)-azanediyl))bis(methylene))-dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

40. The compound of any one of claims 1-38, or a pharmaceutically acceptable salt thereof, wherein R⁶ is a chelating moiety selected from the group consisting of:
DOTA and DO3A; or a radionuclide complex thereof.

41. The compound of any one of claims 1-38, or a pharmaceutically acceptable salt thereof, wherein R⁶ is a chelating moiety selected from the group consisting of: 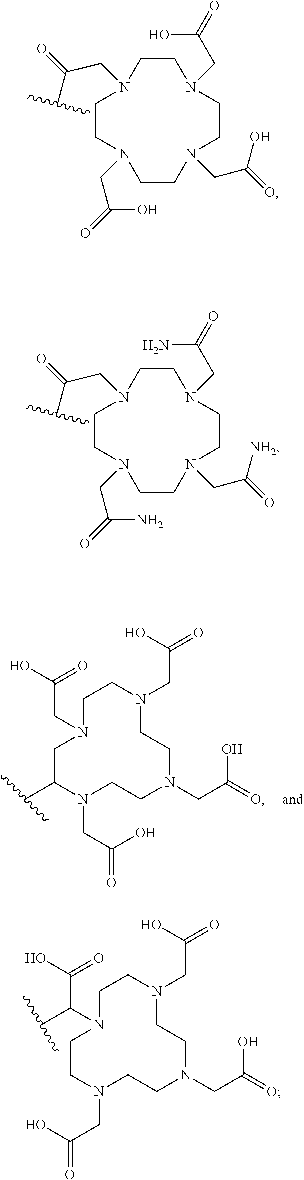 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]
or a radionuclide complex thereof.

42. The compound of any one of claims 1-38, or a pharmaceutically acceptable salt thereof, wherein R⁶ is 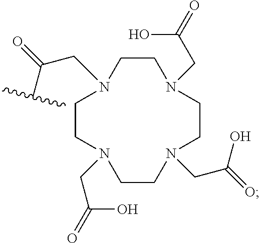 [SMILES: *C(=O)CN1CCN(CC(=O)O)CCN(CC(=O)O)CCN(CC(=O)O)CC1]
or a radionuclide complex thereof.

43. The compound of any one of claims 1-38, or a pharmaceutically acceptable salt thereof, wherein R⁶ is: 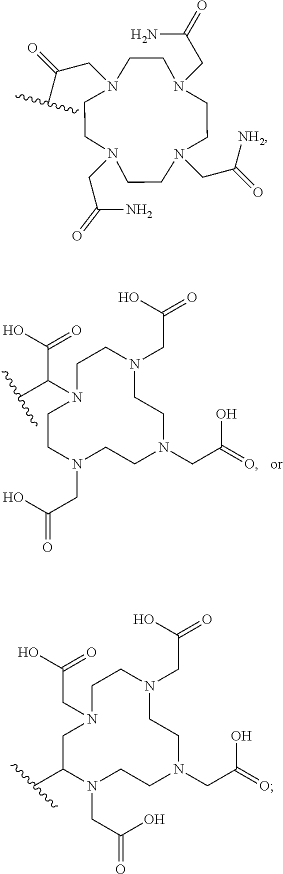 [SMILES: *C(=O)CN1CCN(CC(N)=O)CCN(CC(N)=O)CCN(CC(N)=O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O]
or a radionuclide complex thereof.

44. The compound of any one of claims 1-9, or a pharmaceutically acceptable salt thereof, wherein L^{B} is L^{1.}

45. The compound of any one of claims 10-44, or a pharmaceutically acceptable salt thereof, wherein:
L¹ is -L²-, -L³-, -L⁴-, -L⁵-, -L⁶-, -L⁷-, -L²-L³-, -L²-L⁴-, -L²-L⁷, -L⁴-L⁶-, -L⁴-L⁷-, -L⁶-L⁷-;
L² is absent, substituted or unsubstituted C₁-C₂₀alkylene, substituted or unsubstituted C₁-C₂₀alkylene-NR¹³—, substituted or unsubstituted C₁-C₂₀alkylene-C(═O)—, substituted or unsubstituted C₁-C₂₀alkylene-C(—O)NR¹³—, substituted or unsubstituted C₁-C₂₀alkylene-NR¹³C(═O)—, substituted or unsubstituted 2 to 20 membered heteroalkylene, —(CH₂CH₂O)₂—, —(OCH₂CH₂)₂—, —(CH₂CH₂O)_{w}—CH₂CH₂—, —CH₂CH₂NR¹³—(CH₂CH₂O)_{w}—, —(CH₂CH₂O)_{w}—CH₂CH₂NR¹³—, —CH₂CH₂NR¹³C(═O)—(CH₂CH₂O)_{w}, —(CH₂CH₂O)_{w}—CH₂CH₂NR¹³C(═O)—, —CH₂CH₂C(═O)NR¹³—(CH₂CH₂O)_{w}—, —CH₂CH₂NR¹³C(═O)CH₂—(OCH₂CH₂)_{w} or —(CH₂CH₂O)_{w}—CH₂CH₂C(═O)NR¹³—;
w is 1, 2, 3, 4, 5, or 6;
z is 1, 2, 3, 4, 5, 6, 7, 8, 9, or 10;
L³ is absent, a natural or unnatural amino acid or peptide that is formed from two or more independently selected natural and unnatural amino acids, wherein when two or more amino acids are present then the N atom of the amide linking the amino acids is optionally substituted with C₁-C₆ alkyl;
L⁴ is absent, substituted or unsubstituted 2 to 10 membered heteroalkylene, —CH₂—(OCH₂CH₂)—, —(CH₂CH₂O)—CH₂CH₂—, —(CH₂CH₂O)ᵥCH₂CH₂N R¹⁴C(═O)(CH₂CH₂O)ᵥCH₂CH₂—, —(CH₂CH₂O)ᵥCH₂CH₂C(═O)NR¹⁴(CH₂CH₂O)ᵥCH₂CH₂—, —C(═O)CH₂CH₂, —CH₂CH₂C(═O)—, or C₁-C₆ alkylene that is optionally substituted with 1 or 2 groups independently selected from —OH, —NR¹⁴₂, —CO₂R¹⁴, —O(CH₂CH₂O)ᵤ—CH₃, —NR₁₄ (CH₂CH₂O)ᵤ—CH₃, —NR¹+C(═O)(CH₂CH₂O)ᵤ—CH₃, or —CH₂OCH₂CH₂CO₂R¹⁴;
v is 1, 2, 3, 4, 5, or 6;
u is 1, 2, 3, 4, 5, or 6;
L⁵ is absent, —O—, —S—, —S(═O)—, —S(—O)₂—, —NR¹⁵—, —CH(═NH)—, —CH(═N—NH)—, —CCH₃(═NH)—, —CCH₃ (═N—NH)—, —C(═O)NR¹⁵—, —NR¹⁵C(═O), —NR¹⁵C(═O)O—, —NR¹⁵C(═O)NR¹⁵—, or —OC(═O)NR¹⁵—;
L⁶ is absent or -L⁸-L⁹-L¹⁰-;
L⁸ is absent, —(CH₂)ₜ—, —NR^{w}—, —NR^{w}—(CH₂)ᵥ—, —(CH₂)ₜ—C(═O)—, —C(═O)—(CH₂)ᵥ—, —(CH₂)ᵥ—NR^{w}—, —(CH₂)—NR^{w}C(═O)—, —(CH₂)ₜ—C(═O)NR^{w}—, —CH(NHR^{w})—(CH₂)ₜ—C(═O)—, —NR^{w}C(═O)—(CH₂)ₜ—, and —C(═O)NR^{w}—(CH₂)ᵥ—;
t is 0, 1, 2, or 3;
L¹⁰ is absent, —(CH₂)ᵣ, —NR^{w}—, —NR^{w}—(CH₂)ᵣ, —(CH₂)—C(═O)—, —C(═O)—(CH₂)ᵣ, —(CH₂)—NR^{w}—, —(CH₂)—NR^{w}C(═O)—, —(CH₂)—C(═O)NR^{w}—, —CH(NHR^{w})—(CH₂)ᵣ—C(═O)—, —NR^{w}C(═O)—(CH₂)ᵣ, and —C(═O)NR^{w}—(CH₂)ᵣ—;
r is 0, 1, 2, or 3;
each R^{w} is independently selected from H, C₁-C₆ alkyl, C₁-C₆ alkyl-CO₂H, —(CH₂CH₂O)ₛ—CH₃, —C(═O)—(CH₂CH₂O)ₛ—CH₃, or —(CH₂CH₂O)ₛ—CH₂CH₂CO₂H;
s is 1, 2, 3, 4, 5, or 6;
L¹ is substituted or unsubstituted cycloalkylene, substituted or unsubstituted heterocycloalkylene, substituted or unsubstituted arylene, or substituted or unsubstituted heteroarylene;
each R¹³, R¹⁴, and R¹⁵ are independently selected from H or unsubstituted C₁-C₄ alkyl; and
L⁷ is absent, —NH—, —N(CH₃)—, —O—NH—, substituted or unsubstituted N-heterocycloalkylene, or —O—NH═(substituted or unsubstituted N-heterocycloalkylene).

46. The compound of any one of claims 10-44, or a pharmaceutically acceptable salt thereof, wherein:
L¹ is -L²-, -L³-, -L⁴-, -L⁵-, -L⁶-, -L⁷-, -L²-L³-, -L²-L⁴-, -L²-L⁶-, -L²-L⁷, -L⁴-L⁶-, -L⁴-L⁷-, —L⁶-L⁷-, -L²-L⁴-L⁷, -L²-L⁵L⁷-L²L⁶-L⁷, -L³-L⁴ L⁷,L³-L⁵L⁷-, or -L²-L³-L⁴-L⁵-L⁶ L⁷-;
L² is absent, substituted or unsubstituted C₁-C₂₀ alkylene, substituted or unsubstituted C₁-C₂₀ alkylene-NH—, substituted or unsubstituted C₁-C₂₀ alkylene-C(═O)—, substituted or unsubstituted C₁-C₂₀ alkylene-C(═O)NH—, substituted or unsubstituted C₁-C₂₀ alkylene-NHC(—O)—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NH—(CH₂CH₂O)_{w}—, —(CH₂CH₂O)_{w}—CH₂CH₂NH—, —CH₂CH₂NHC(═O)—(CH₂CH₂O)_{w}, —(CH₂CH₂O)_{w}—CH₂CH₂NHC(═O)—, —CH₂CH₂C(═O)NH—(CH₂CH₂O)_{w}—, —CH₂CH₂NHC(═O)CH₂—(OCH₂CH₂)_{w} or —(CH₂CH₂O)_{w}—CH₂CH₂C(═O)NH—;
L³ is absent, a natural or unnatural amino acid or peptide that is formed from one or more independently selected natural and unnatural amino acids, wherein when two or more amino acids are present then the N atom of the amide linking the amino acids is optionally substituted with C₁-C₆ alkyl;
L⁴ is —CH₂—(OCH₂CH₂)—, —(CH₂CH₂O)—CH₂CH₂—, —(CH₂CH₂O)_{w}CH₂CH₂NHC(═O)(CH₂CH₂O)ᵥCH₂CH₂—, —(CH₂CH₂O)ᵥCH₂CH₂C(═O)NH(CH₂CH₂O)_{w}CH₂CH₂—, —C(═O)CH₂CH₂, —CH₂CH₂C(═O)—, or C₁-C₆ alkylene that is optionally substituted with 1 or 2 groups independently selected from —OH, —NH₂, —CO₂H, —O(CH₂CH₂O)ᵤ—CH₃, —NH(CH₂CH₂O)ᵤ—CH₃, —NHC(═O)(CH₂CH₂O)ᵤ—CH₃, and —CH₂OCH₂CH₂CO₂H;
L⁵ is absent, —C(═O)NH—, or —NHC(═O)—;
L¹ is absent or -L⁸-L⁹-L¹⁰-;
L⁸ is absent, —(CH₂)ₜ—, —NR^{w}—(CH₂)—, —(CH₂)ₜ—C(═O)—, —C(═O)—(CH₂)ₜ—, —(CH₂)ₜ—NR^{w}—, —(CH₂)ₜ—NR^{w}C(═O)—, —(CH₂)ₜ—C(═O)NR^{w}—, —CH(NHR^{w})—(CH₂)ₜ—C(═O)—, —NR^{w}C(═O)—(CH₂)ₜ—, and —C(═O)NR^{w}—(CH₂)ᵥ—;
L¹⁰ is absent, —(CH₂)ᵣ, —NR^{w}—(CH₂)ᵣ, or —C(═O)—(CH₂)ᵣ—;
r is 0, 1, 2, or 3;
each R^{w} is independently selected from H, C₁-C₆ alkyl, C₁-C₆ alkylCO₂H, (CH₂CH₂O)ₛ—CH₃, —C(═O)—(CH₂CH₂O)ₛ—CH₃, or —(CH₂CH₂O)ₛ—CH₂CH₂CO₂H;
L⁹ is substituted or unsubstituted cycloalkylene, substituted or unsubstituted heterocycloalkylene, substituted or unsubstituted arylene, or substituted or unsubstituted heteroarylene; and
L⁷ is absent, —NH—, —N(CH₃)—, —O—NH—, substituted or unsubstituted N-heterocycloalkylene, or —O—NH═(substituted or unsubstituted N-heterocycloalkylene).

47. The compound of claim 45 or 46, or a pharmaceutically acceptable salt thereof, wherein: L¹ is -L²- or -L²-L⁷.

48. The compound of claim 45 or 46, or a pharmaceutically acceptable salt thereof, wherein: L¹ is -L³- or -L²-L³.

49. The compound of claim 48, wherein L² is substituted or unsubstituted C₁-C₆ alkylene-C(═O)— and L³ is a peptide that is formed from one or more independently selected natural and unnatural amino acids.

50. The compound of any one of claims 45-48, or a pharmaceutically acceptable salt thereof, wherein L² is absent.

51. The compound of any one of claims 45-48, or a pharmaceutically acceptable salt thereof, wherein L² is —CH₂— or —CH₂CH₂—.

52. The compound of any one of claims 45-48, or a pharmaceutically acceptable salt thereof, wherein L² is substituted or unsubstituted C₁-C₂₀alkylene-NH—, substituted or unsubstituted C₁-C₂₀alkylene-C(═O)—, substituted or unsubstituted C₁-C₂₀alkylene-C(═O)NH—, or substituted or unsubstituted C₁-C₂₀alkylene-NHC(═O)—.

53. The compound of any one of claims 45-48, or a pharmaceutically acceptable salt thereof, wherein L² is —(CH₂CH₂O)_{w}—CH₂CH₂—, —(CH₂CH₂O)—CH₂CH₂NH—, —CH₂CH₂NHC(═O)—(CH₂CH₂O)_{w}—, —(CH₂CH₂O)_{w}—CH₂CH₂NHC(═O)—, —CH₂CH₂C(═O)NH—(CH₂CH₂O)_{w}—, or —CH₂CH₂NHC(═O)CH₂—(OCH₂CH₂)_{w}—.

54. The compound of any one of claims 45-48, or a pharmaceutically acceptable salt thereof, wherein L² is —(CH₂CH₂O)_{w}—CH₂CH₂— or —(CH₂CH₂O)_{w}—CH₂CH₂NH—.

55. The compound of any one of claims 45-54, or a pharmaceutically acceptable salt thereof, wherein L³ is absent.

56. The compound of any one of claims 45-54, or a pharmaceutically acceptable salt thereof, wherein L³ is a natural amino acid, an unnatural amino acid, or peptide that is formed from two or more independently selected amino acids selected from the group consisting of alanine (Ala), Ala(SO₃H), arginine (Arg), asparagine (Asn), aspartate (Asp), cysteine (Cys), glutamine (Gln), glutamate (Glu), glycine (Gly), leucine (Leu), lysine(Lys), methionine (Met), phenylalanine (Phe), proline (Pro), serine (Ser), tyrosine (Tyr), and valine (Val), wherein when two or more amino acids are present then the N atom of the amide linking the amino acids is optionally substituted with —CH₃.

57. The compound of claim 45-54, or a pharmaceutically acceptable salt thereof, wherein L³ is a peptide that is: Ala-Lys-Ser-Asn-Asn-Ser-Ala-NH₂, Ala-Ser-Asn-Lys-Asn-Ser-Ala-NH₂, Ala-Ser-Asn-Asn-Ser-Lys-Ala-NH₂, Ala-Arg-Arg-Lys-Glu-Glu-Glu-NH₂, or Ala-Glu-Ala-Lys-Glu-Ala-NH₂.

58. The compound of any one of claims 45-57, or a pharmaceutically acceptable salt thereof, wherein L⁴ is absent.

59. The compound of any one of claims 45-57, or a pharmaceutically acceptable salt thereof, wherein L⁴ is —C(═O)CH₂CH₂—.

60. The compound of any one of claims 45-57, or a pharmaceutically acceptable salt thereof, wherein L⁴ is unsubstituted C₁-C₆ alkylene.

61. The compound of any one of claims 45-57, or a pharmaceutically acceptable salt thereof, wherein L⁴ is C₁-C₆ alkylene that is substituted with 1 or 2 groups independently selected from —OH, —NH₂, —CO₂H, or —CH₂OCH₂CH₂CO₂H.

62. The compound of any one of claims 45-61, or a pharmaceutically acceptable salt thereof, wherein L⁵ is absent.

63. The compound of any one of claims 45-61, or a pharmaceutically acceptable salt thereof, wherein L⁵ is —C(═O)NH—.

64. The compound of any one of claims 45-61, or a pharmaceutically acceptable salt thereof, wherein L⁵ is —NHC(═O)—.

65. The compound of any one of claims 45-64, or a pharmaceutically acceptable salt thereof, wherein L⁹ is a substituted or unsubstituted C₄-C₈ cycloalkylene.

66. The compound of any one of claims 45-64, or a pharmaceutically acceptable salt thereof, wherein L⁹ is a substituted or unsubstituted 3 to 8 membered heterocycloalkylene.

67. The compound of any one of claims 45-64, or a pharmaceutically acceptable salt thereof, wherein L⁹ is azetidinylene, pyrrolidinylene, piperidinylene or piperazinylene.

68. The compound of any one of claims 45-64, or a pharmaceutically acceptable salt thereof, wherein L⁹ is a monosaccharide.

69. The compound of any one of claims 45-64, or a pharmaceutically acceptable salt thereof, wherein L⁹ is

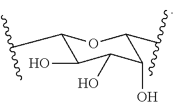 [SMILES: *C1OC(*)C(O)C(O)C1O]

70. The compound of any one of claims 45-64, or a pharmaceutically acceptable salt thereof, wherein L⁹ is a 7 to 12 membered spirocyclic heterocycloalkylene.

71. The compound of any one of claims 45-64, or a pharmaceutically acceptable salt thereof, wherein L¹ is

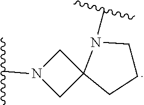 [SMILES: *N1CC2(CCCN2*)C1]

72. The compound of any one of claims 45-64, or a pharmaceutically acceptable salt thereof, wherein L¹ is

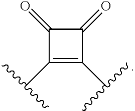 [SMILES: *c1c(*)c(=O)c1=O]

73. The compound of any one of claims 45-72, or a pharmaceutically acceptable salt thereof, wherein L⁸ is absent.

74. The compound of any one of claims 45-72, or a pharmaceutically acceptable salt thereof, wherein L⁸ is —(CH₂)—, —(CH₂)ₜ—C(═O)NR^{w}—, or —CH(NHR^{w})—(CH₂)ₜ—C(═O)—.

75. The compound of any one of claims 45-74, or a pharmaceutically acceptable salt thereof, wherein L¹⁰ is absent.

76. The compound of any one of claims 45-74, or a pharmaceutically acceptable salt thereof, wherein L¹⁰ is —(CH₂)ᵣ, —NR^{w}—(CH₂)ᵣ, or —C(═O)—(CH₂)ᵣ.

77. The compound of claim 76, or a pharmaceutically acceptable salt thereof, wherein ris 1.

78. The compound of any one of claims 45-77, or a pharmaceutically acceptable salt thereof, wherein L¹ is absent.

79. The compound of any one of claims 45-77, or a pharmaceutically acceptable salt thereof, wherein L¹ is —NH—.

80. The compound of any one of claims 45-77, or a pharmaceutically acceptable salt thereof, wherein L⁷ is substituted or unsubstituted N-heterocycloalkylene.

81. The compound of any one of claims 45-77, or a pharmaceutically acceptable salt thereof, wherein L⁷ is

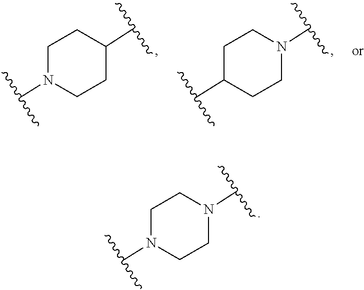 [SMILES: *C1CCN(*)CC1 ; *C1CCN(*)CC1 ; *N1CCN(*)CC1]

82. The compound of any one of claim 45 or 46, or a pharmaceutically acceptable salt thereof, wherein L¹ is -L²-;
L² is —(CH₂CH₂O)_{w}—CH₂CH₂NH—; and w is 1, 2, 3, 4, 5, or 6.

83. The compound of claim 45 or 46, or a pharmaceutically acceptable salt thereof, wherein L¹ is -L³-; and
L³ is a natural amino acid, an unnatural amino acid, or peptide.

84. The compound of claim 45 or 46, or a pharmaceutically acceptable salt thereof, wherein L¹ is -L⁶-;
L¹ is -L⁸-L⁹-L¹⁰-;
L⁸ is —(CH₂)—C(═O)NR^{w}—; L¹ is substituted or unsubstituted heterocycloalkylene; L¹⁰ is absent; and tis 1, 2, or 3.

85. The compound of claim 84, or a pharmaceutically acceptable salt thereof, wherein L¹ is azetidine, pyrrolidine, piperidine, or piperazine.

86. The compound of claim 84 or 85, or a pharmaceutically acceptable salt thereof, wherein R^{w} is C₁-C₆ alkyl-CO₂H.

87. The compound of claim 84 or 85, or a pharmaceutically acceptable salt thereof, wherein R^{w is —(CH}₂CH₂O)ₛ—CH₂CH₂CO₂H.

88. The compound of claim 45 or 46, or a pharmaceutically acceptable salt thereof, wherein L¹ is -L⁷-; and L¹ is substituted or unsubstituted N-heterocycloalkylene.

89. The compound of claim 45 or 46, or a pharmaceutically acceptable salt thereof, wherein L¹ is -L²-L³-;
L² is substituted or unsubstituted C₁-C₆ alkylene-NH—, substituted or unsubstituted C₁-C₆ alkylene-C(—O)—, substituted or unsubstituted C₁-C₆ alkylene-NH—, or —(CH₂CH₂O)_{w}—CH₂CH₂NH—; and
L³ is a natural or unnatural amino acid or peptide.

90. The compound of claim 89, wherein L³ is a peptide.

91. The compound of claim 45 or 46, or a pharmaceutically acceptable salt thereof, wherein L¹ is -L²-L⁴-;
L² is —(CH₂CH₂O)_{w}—CH₂CH₂NHC(═O)—; and
L⁴ is unsubstituted C₁-C₆ alkylene.

92. The compound of claim 45 or 46, or a pharmaceutically acceptable salt thereof, wherein L¹ is -L²-L⁷-;
L² is substituted or unsubstituted C₁-C₆ alkylene-, substituted or unsubstituted C₁-C₆ alkylene-C(═O)NH—, —(CH₂CH₂O)_{w}—CH₂CH₂—; and
L¹ is —NH—, —O—NH—, or substituted or unsubstituted N-heterocycloalkylene.

93. The compound of claim 45 or 46, or a pharmaceutically acceptable salt thereof, wherein L¹ is -L²-L⁷-;
L² is —(CH₂CH₂O)_{w}—CH₂CH₂—; and
L¹ is —NH—.

94. The compound of claim 45 or 46, or a pharmaceutically acceptable salt thereof, wherein
L¹ is -L⁴-L⁶-;
L⁴ is C₁-C₆ alkylene that is optionally substituted with 1 or 2 groups independently selected from —OH or —NH₂;
L⁶ is -L⁸-L⁹-L¹⁰-;
L⁸ is —(CH₂)—C(═O)NR^{w}—; R^{w} is H; L¹ is substituted or unsubstituted heterocycloalkylene; and L¹⁰ is absent.

95. The compound of claim 45 or 46, or a pharmaceutically acceptable salt thereof, wherein
L¹ is -L⁴-L¹-;
L⁴ is C₁-C₆ alkylene that is optionally substituted with 1 or 2 groups independently selected from —OH or —NH₂; and
L¹ is —NH—.

96. The compound of claim 45 or 46, or a pharmaceutically acceptable salt thereof, wherein L¹ is -L⁶-L⁷-;
L⁶ is -L⁸-L⁹-L¹⁰-;
L⁸ is absent, —CH(NHR^{w})—(CH₂)ₜ—C(═O)—, or —(CH₂)—C(═O)NR^{w}—; L¹ is substituted or unsubstituted heterocycloalkylene; L¹⁰ is absent, —(CH₂)ᵣ, or —C(═O)—(CH₂)ᵣ—; and
L⁷ is —NH— or substituted or unsubstituted N-heterocycloalkylene.

97. The compound of claim 96, or a pharmaceutically acceptable salt thereof, wherein R^{w is —C(═O)—(CH}₂CH₂O)ₛ—CH₃ or —(CH₂CH₂O)ₛ—CH₂CH₂CO₂H.

98. The compound of claim 45 or 46, or a pharmaceutically acceptable salt thereof, wherein L¹ is -L²-L^{A}-L^{7;}
L² is substituted or unsubstituted C₁-C₆ alkylene-NHC(═O)—, —(CH₂CH₂O)_{z}—, or —CH₂CH₂NHC(═O)—(CH₂CH₂O)_{w};
L⁴ is —CH₂—(OCH₂CH₂)ᵥ— or C₁-C₆ alkylene that is optionally substituted with 1 or 2 groups independently selected from —OH, —NH₂, or —CH₂OCH₂CH₂CO₂H; and
L⁷ is —NH— or substituted or unsubstituted N-heterocycloalkylene.

99. The compound of claim 45 or 46, or a pharmaceutically acceptable salt thereof, wherein L¹ is -L²-L⁷-;
L² is substituted or unsubstituted C₁-C₆ alkylene; and
L⁷ is —O—N═ (substituted or unsubstituted N-heterocycloalkylene).

100. The compound of claim 45 or 46, or a pharmaceutically acceptable salt thereof, wherein L¹ is -L²-L⁶-L⁷-;
L² is substituted or unsubstituted C₁-C₆ alkylene-NH—, substituted or unsubstituted C₁-C₆ alkylene-NHC(═O)—, substituted or unsubstituted C₁-C₆ alkylene-C(═O)NH, or
(CH₂CH₂O)_{w}—CH₂CH₂NHC(═O)—;
L⁶ is -L⁸-L⁹-L¹⁰-;
L⁸ is absent or —(CH₂)ₜ; L⁹ is substituted or unsubstituted heterocycloalkylene; L¹⁰ is absent, —(CH₂)ᵣ, or —NR^{w}—(CH₂)ᵣ—; and
L⁷ is —NH—.

101. The compound of claim 45 or 46, or a pharmaceutically acceptable salt thereof, wherein L¹ is L³-L⁴-L⁷-;
L³ is a peptide, wherein the N atom of the amide linking the amino acids is substituted with α-CH₃;
L⁴ is —C(═O)CH₂CH₂—; and
L⁷ is —NH—.

102. The compound of claim 45 or 46, or a pharmaceutically acceptable salt thereof, wherein L¹ is L⁴-L⁵-L⁷-;
L⁴ is a C₁-C₆ alkylene that is optionally substituted with 1 or 2 groups independently selected from —OH or —NH₂;
L⁵ is —C(═O)NH—; and
L⁷ is substituted or unsubstituted N-heterocycloalkylene.

103. The compound of claim 10, or a pharmaceutically acceptable salt thereof, wherein R is —(CH₂CH₂O)ᵤ—CH₂CH₂N(L¹-R⁶)₂—.

104. The compound of claim 10, or a pharmaceutically acceptable salt thereof, wherein R is —CH-(L¹R⁶) 2.

105. The compound of claim 103, or a pharmaceutically acceptable salt thereof, wherein L¹ is L²; L² is —(CH₂CH₂O)_{w}—CH₂CH₂NH—; and w is 3.

106. The compound of claim 104, or a pharmaceutically acceptable salt thereof, wherein L¹ is L²-L⁴-L^{7;}
L² is substituted or unsubstituted C₁-C₂₀alkylene-NHC(═O)—;
L⁴ is C₁-C₆ alkylene; and
L⁷ is NH.

107. The compound of any one of claims 10-81, or a pharmaceutically acceptable salt thereof, wherein L¹ is:

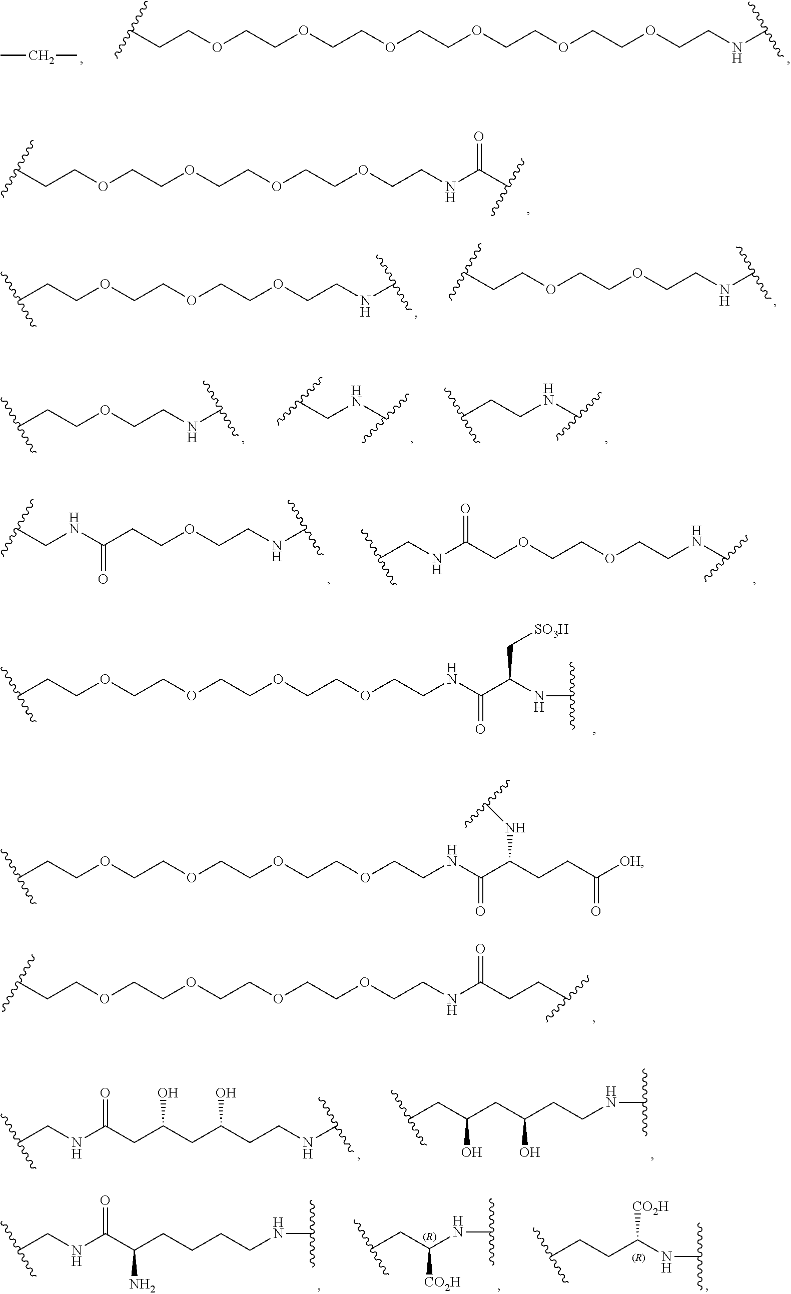

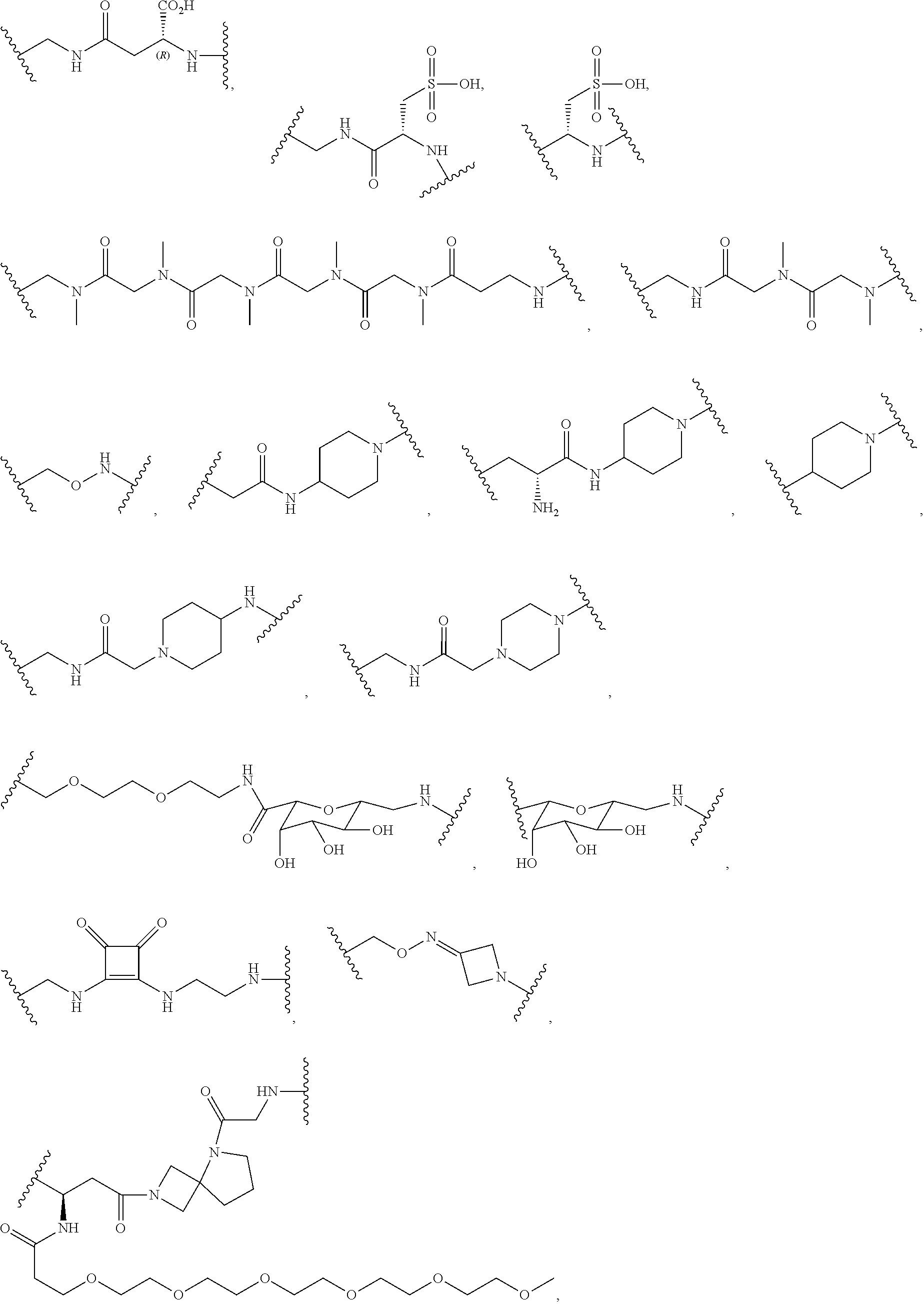

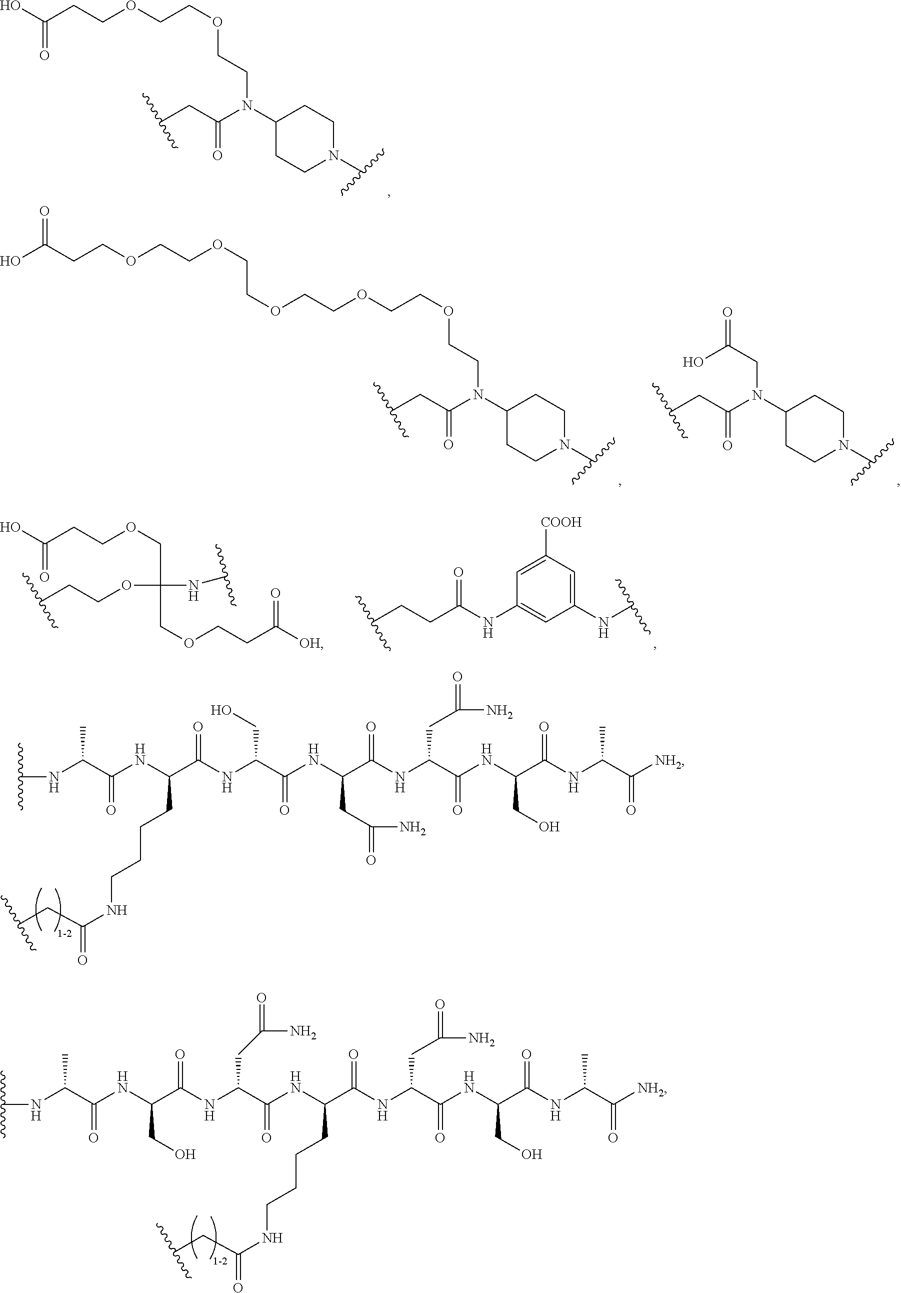

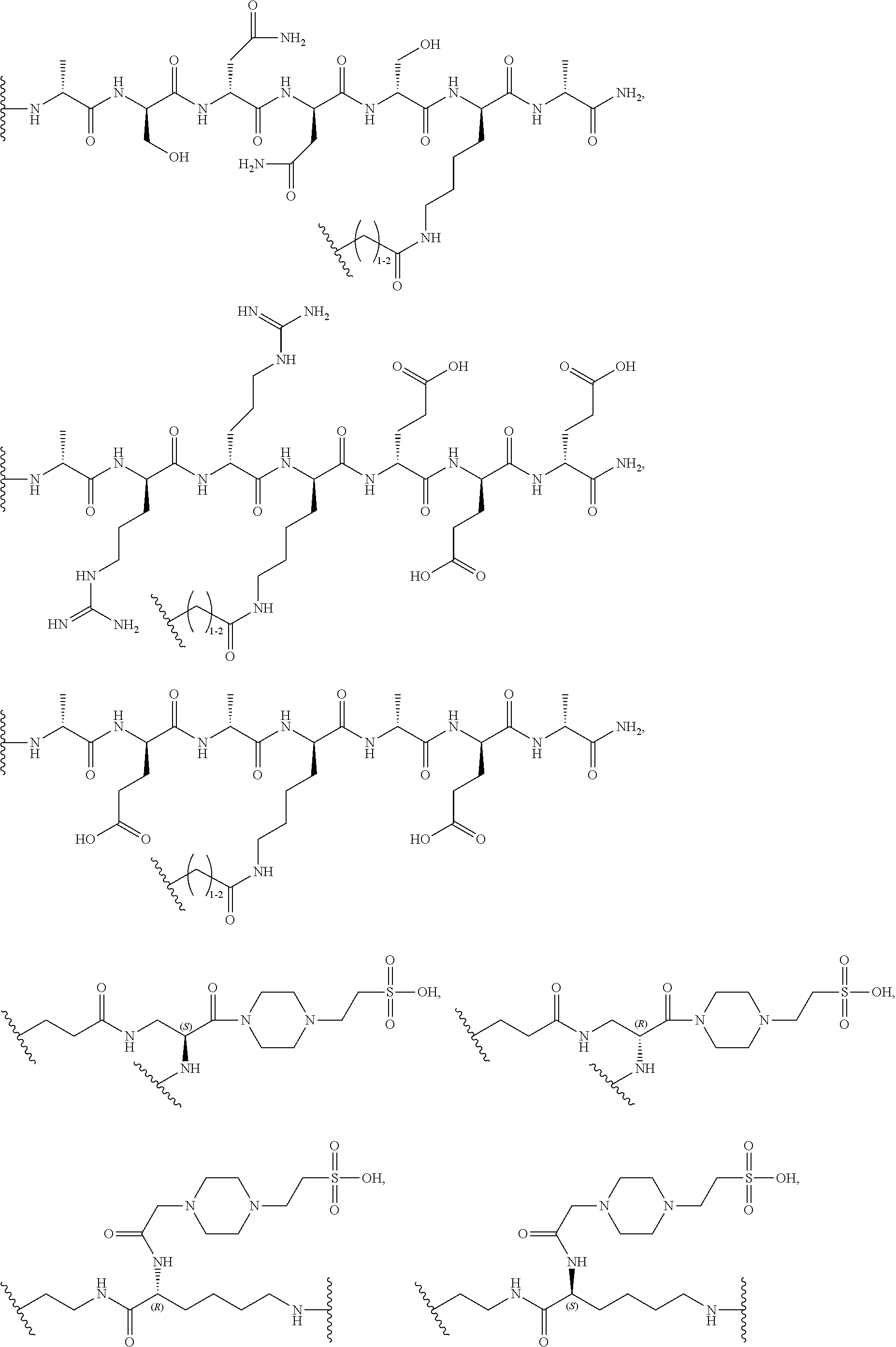

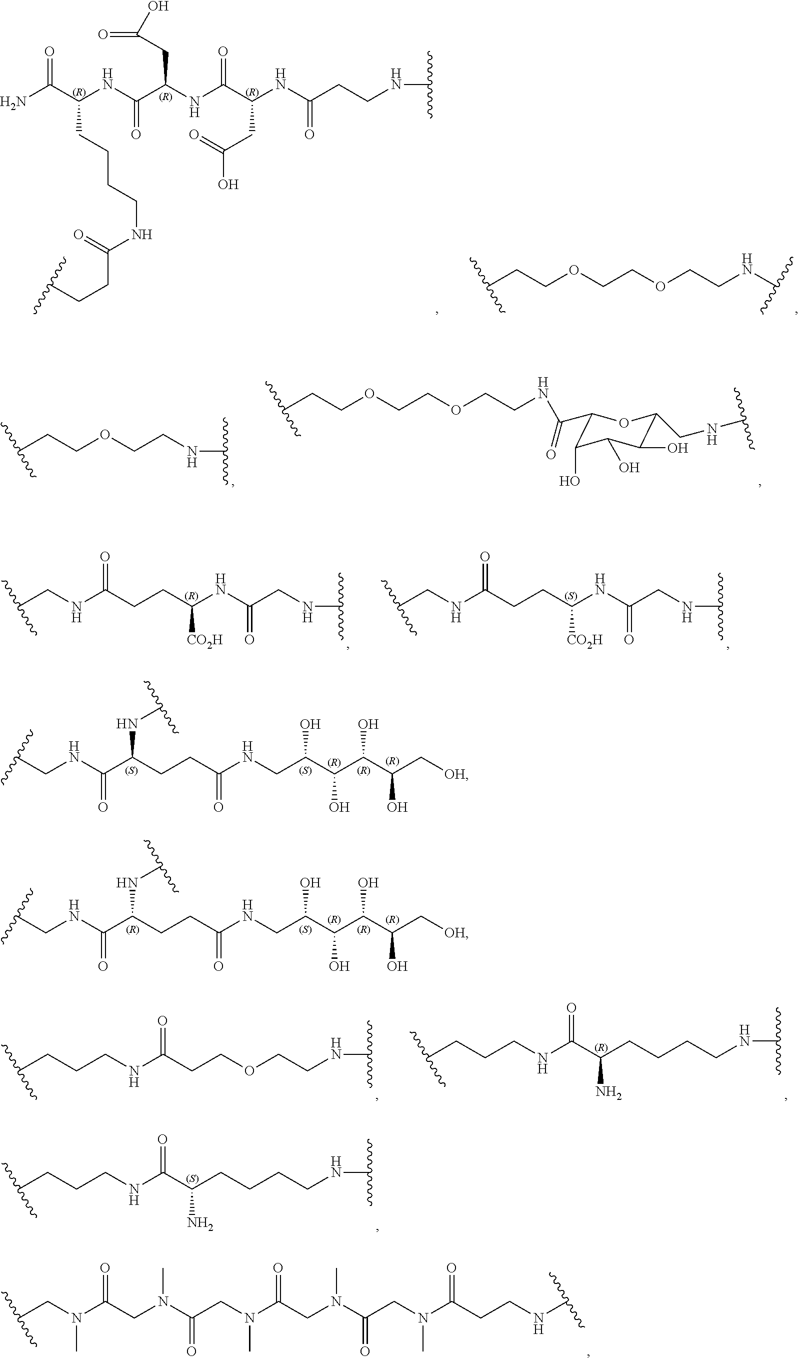

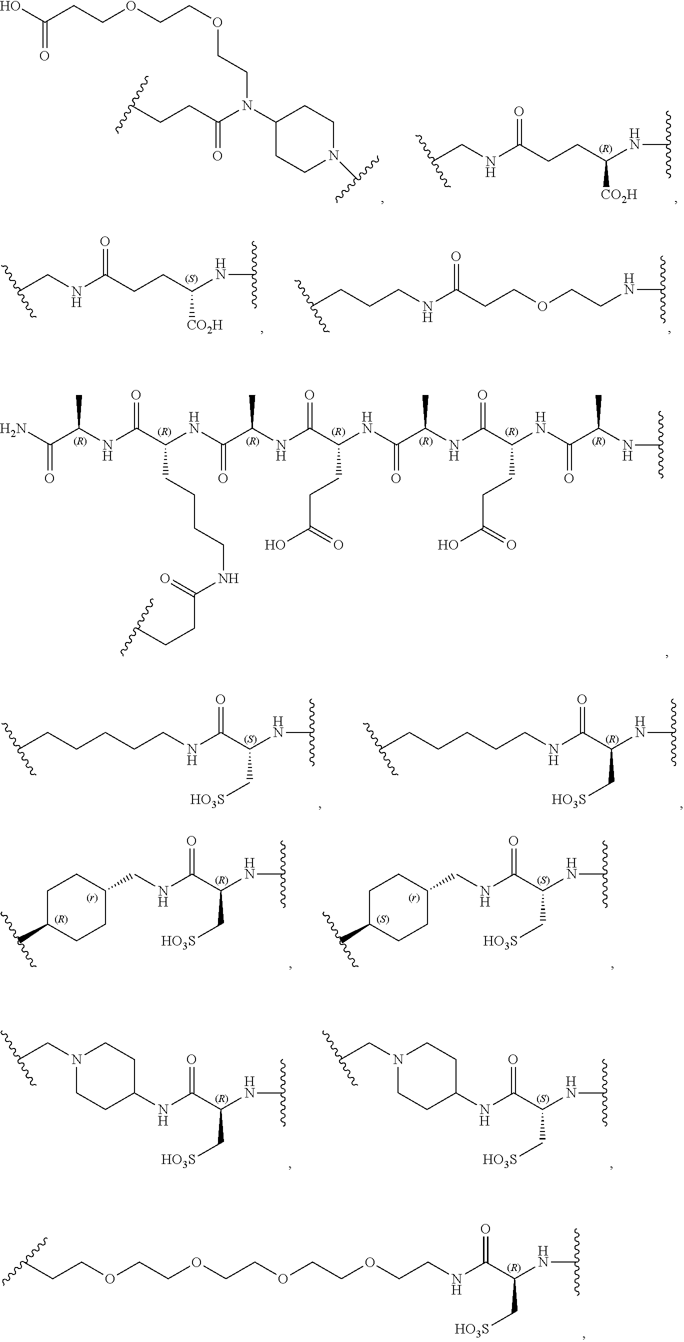

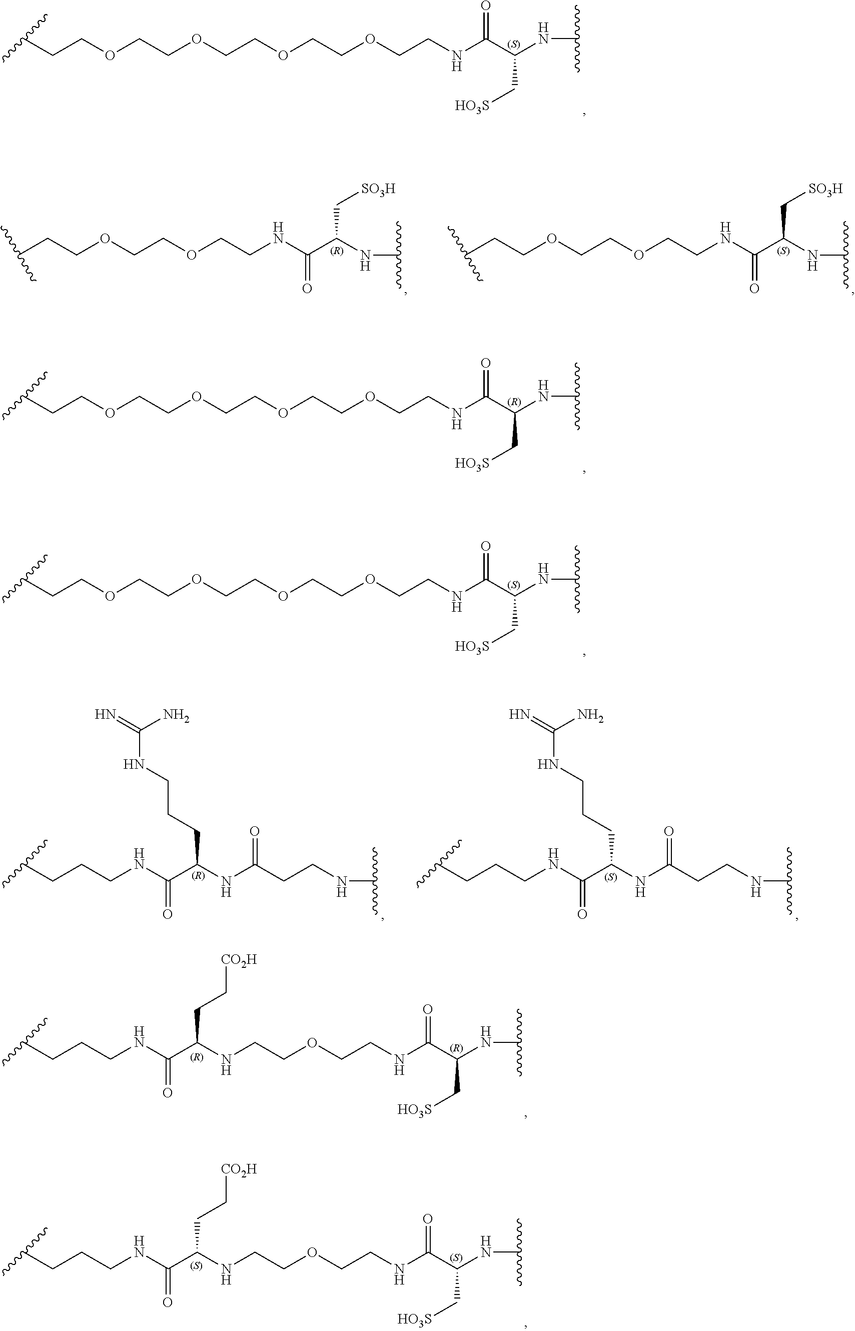

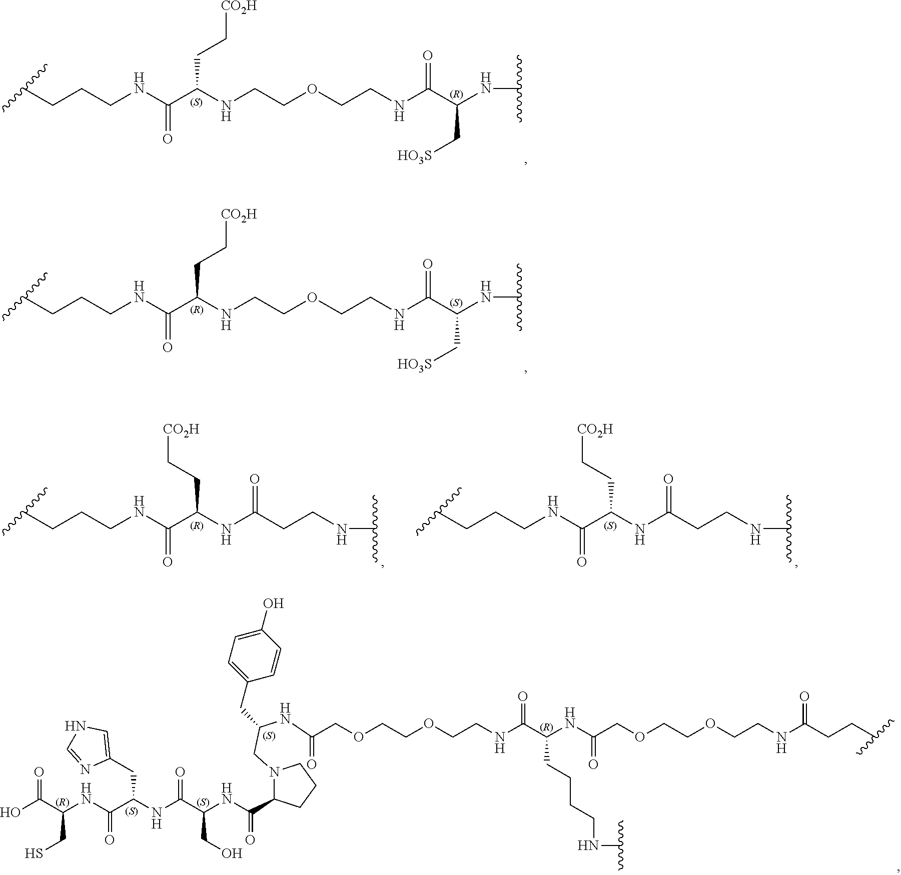

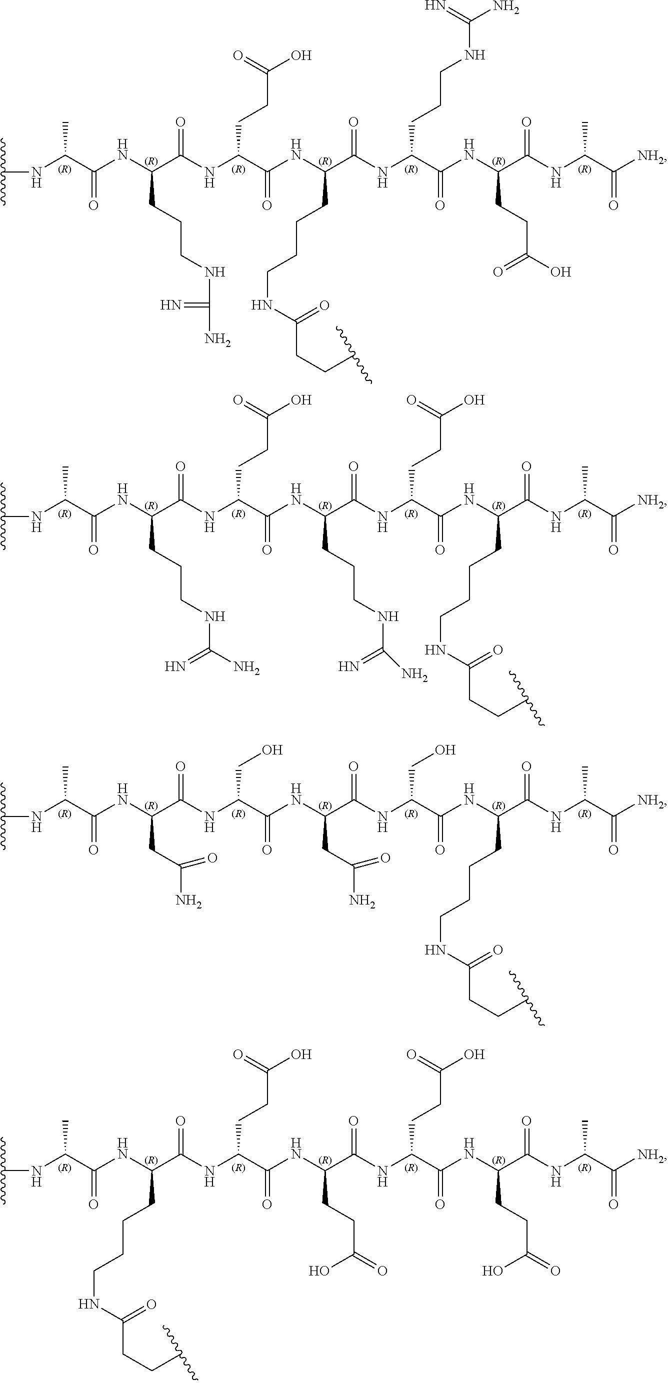

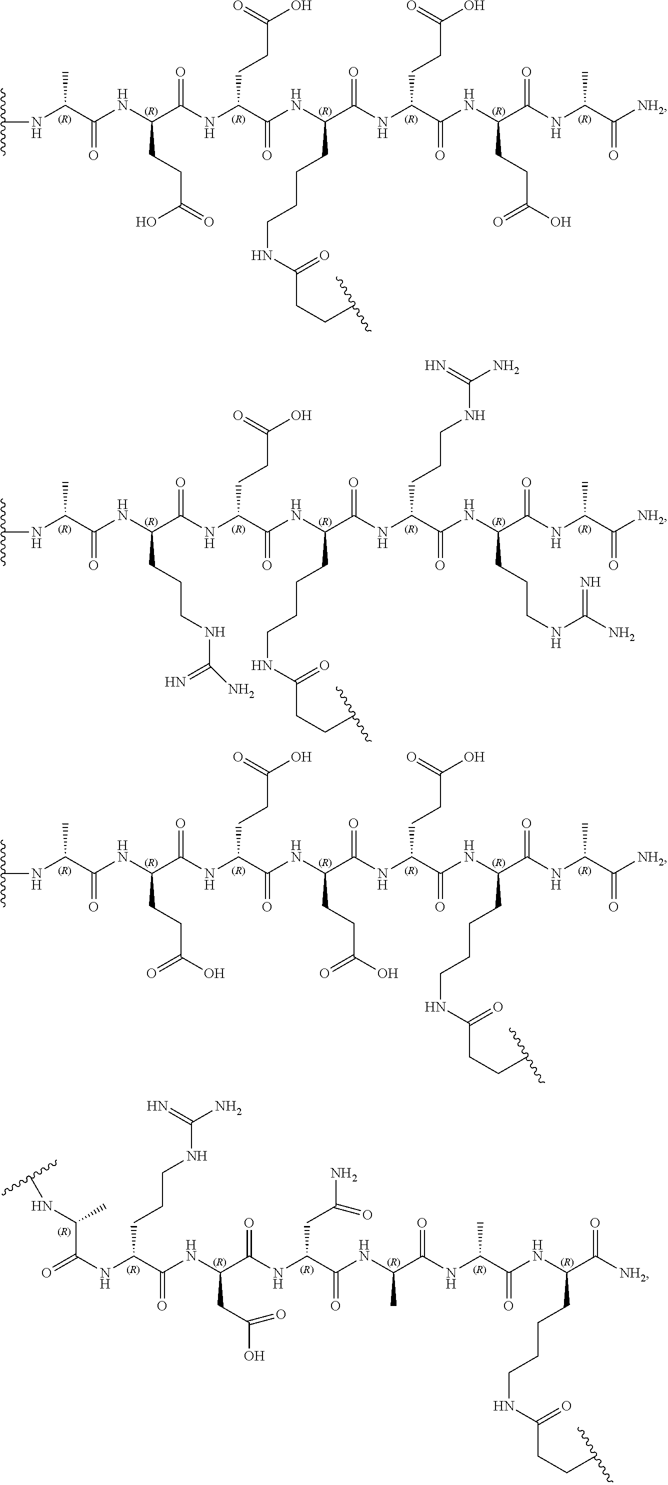

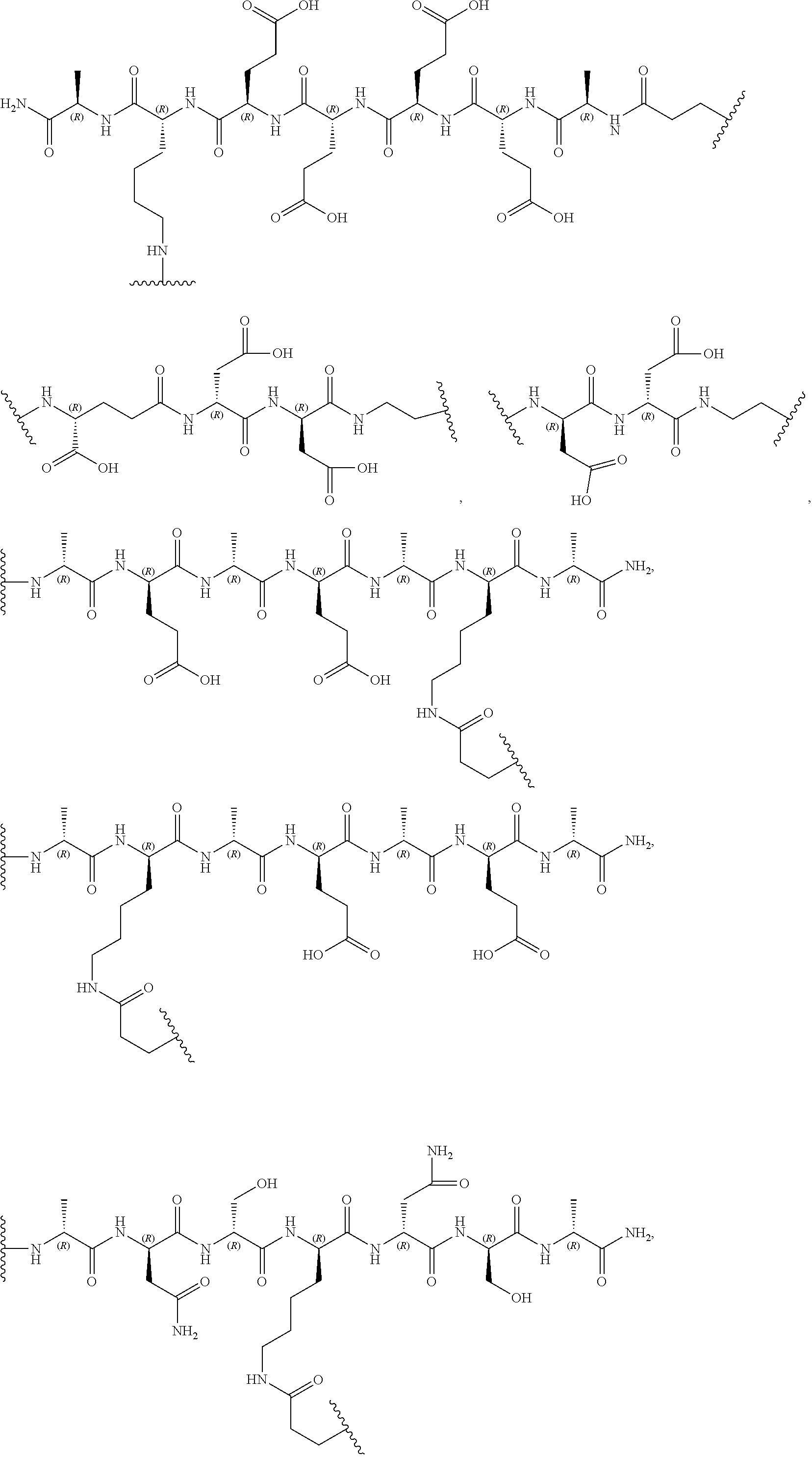

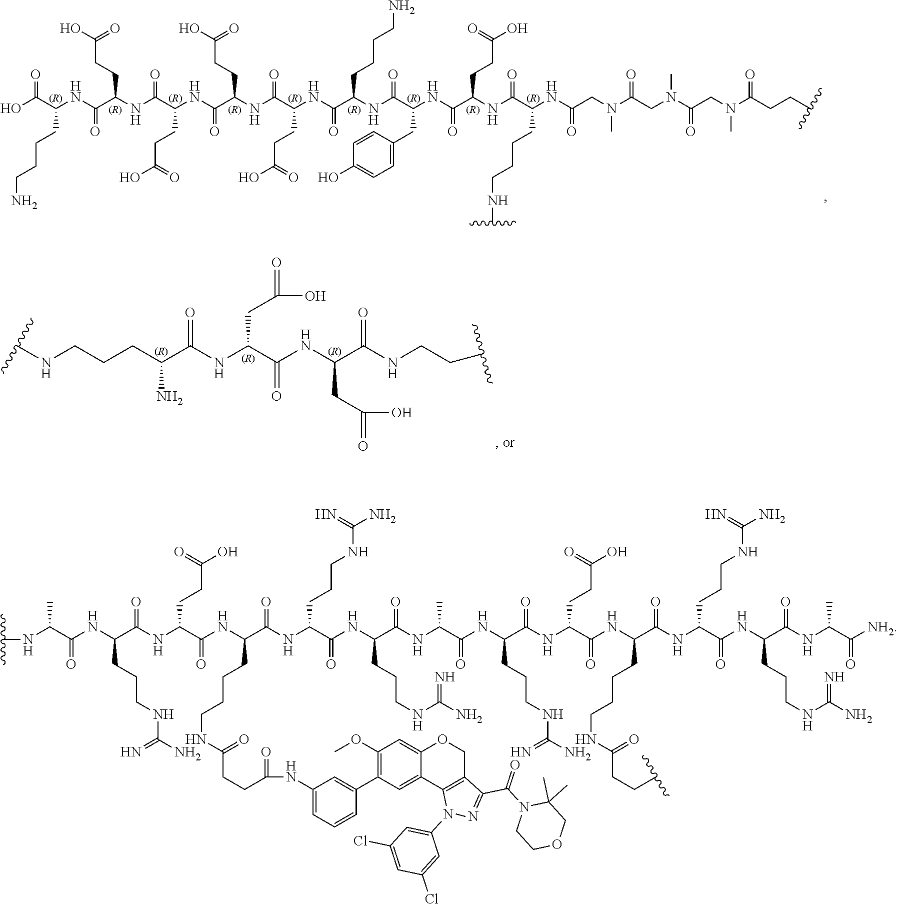

108. The compound of any one of claims 11-43, or a pharmaceutically acceptable salt thereof, wherein-L¹-R⁶ is:

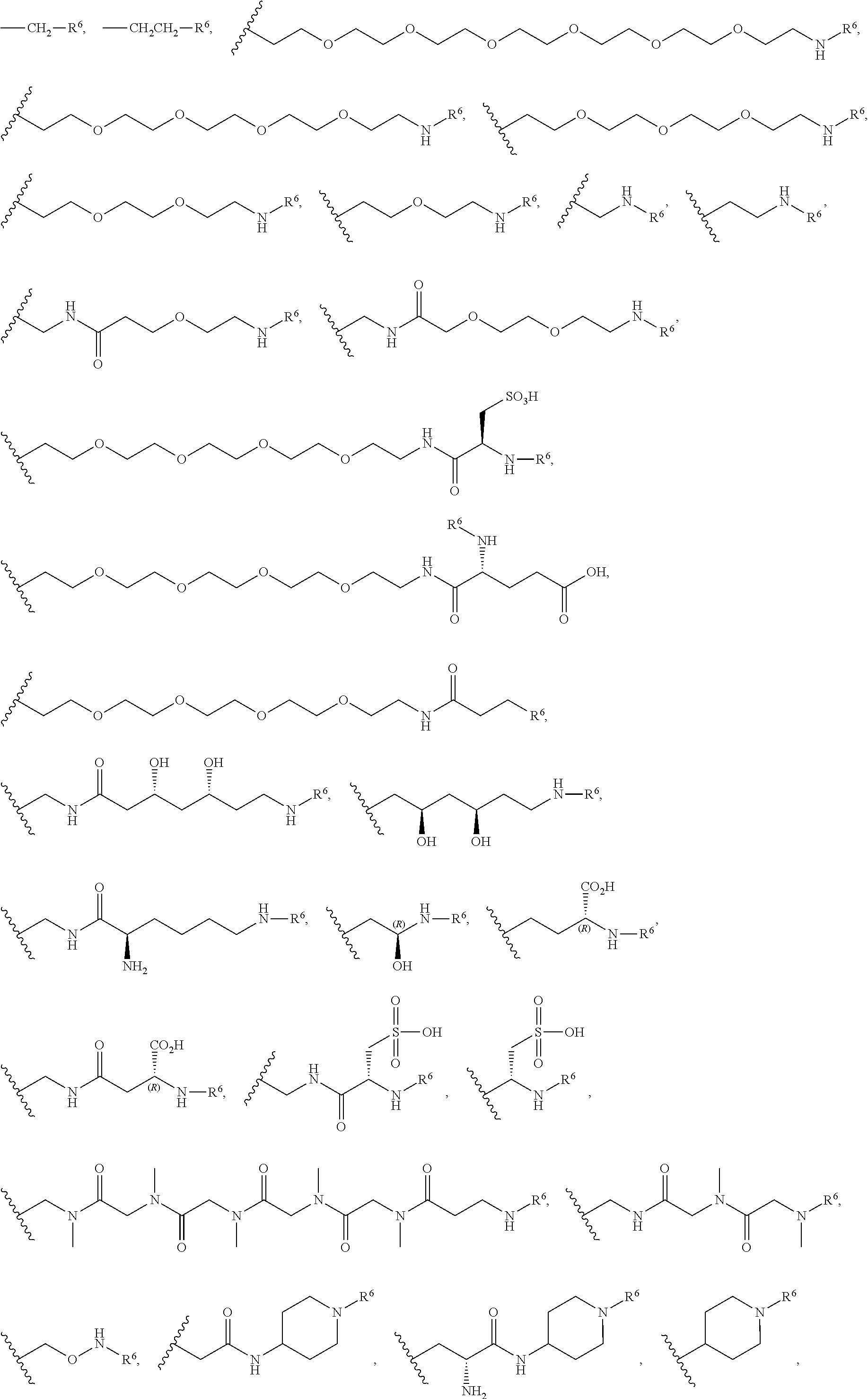

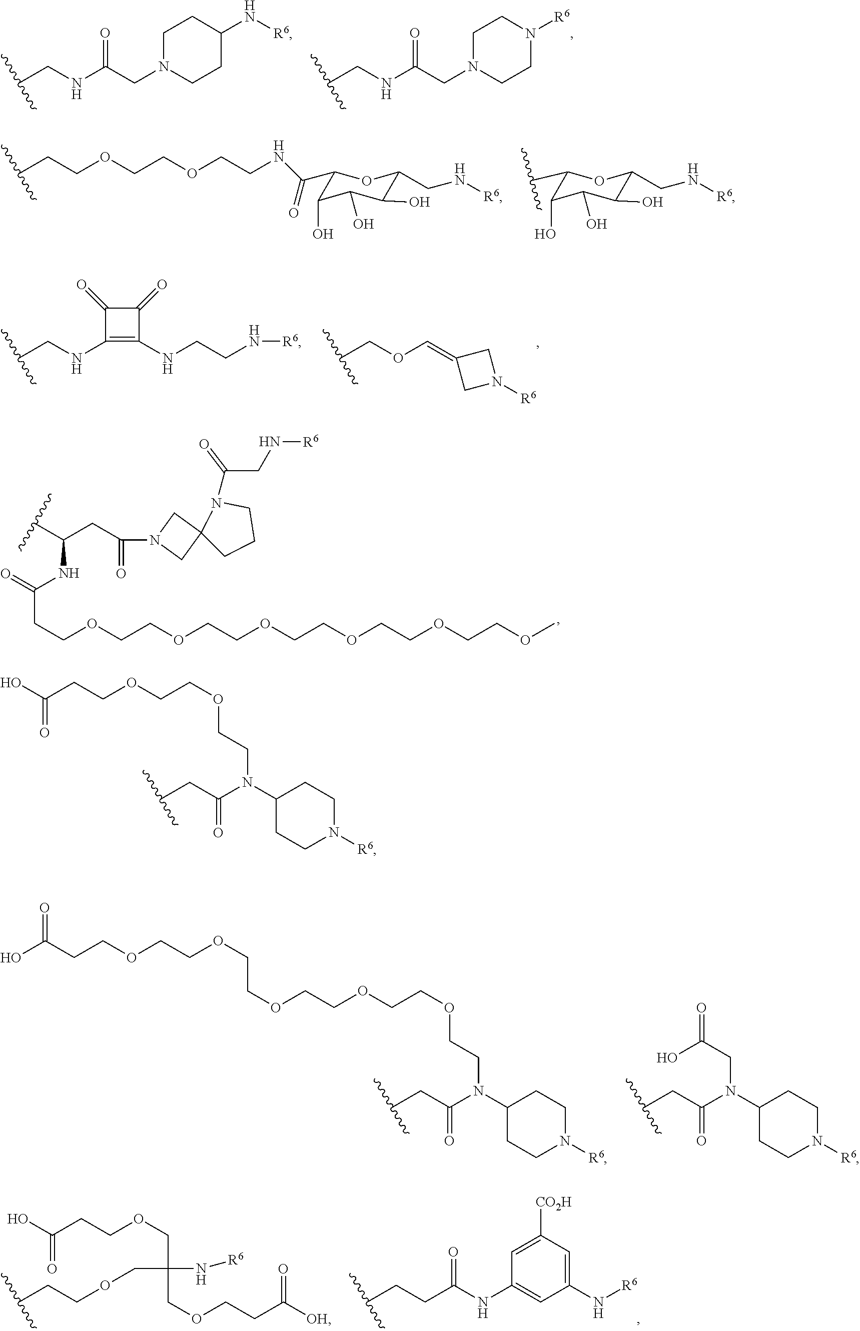

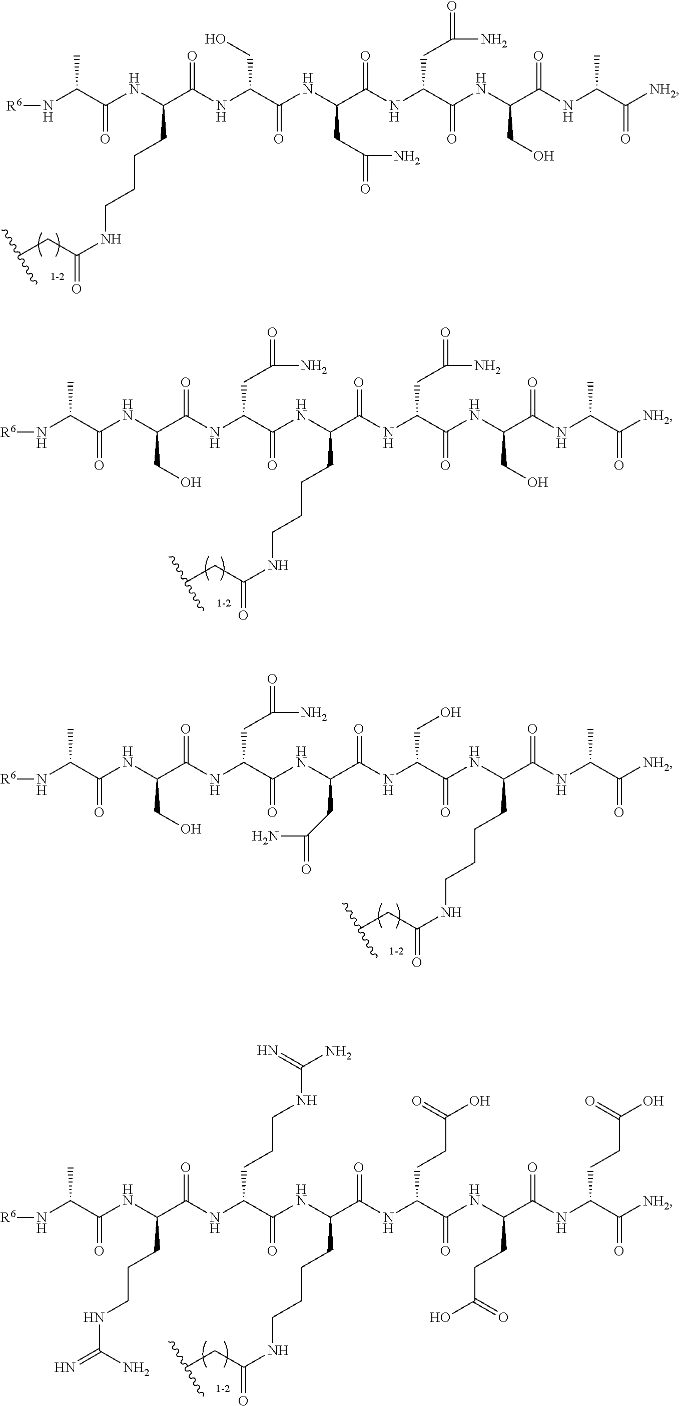

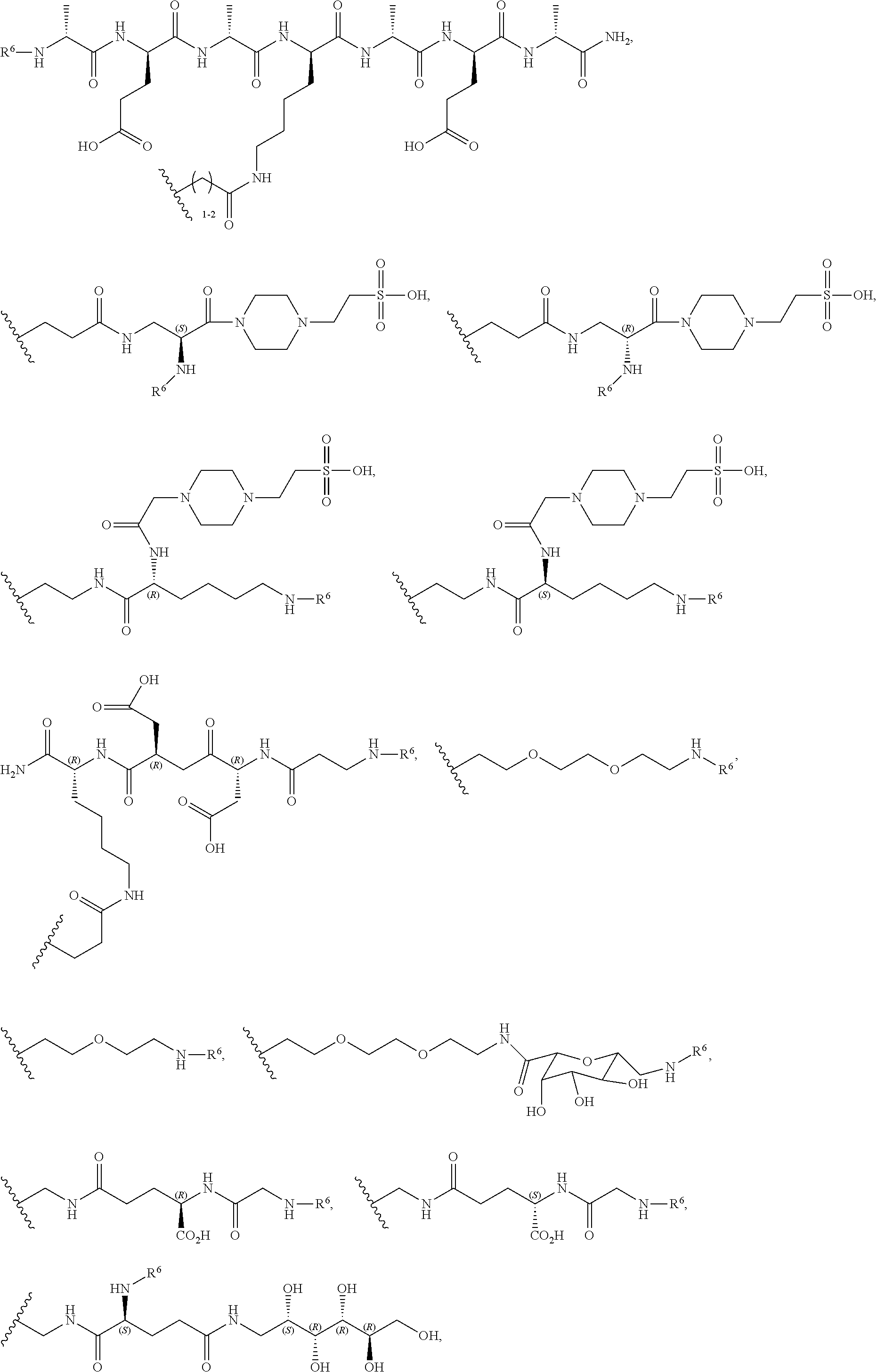

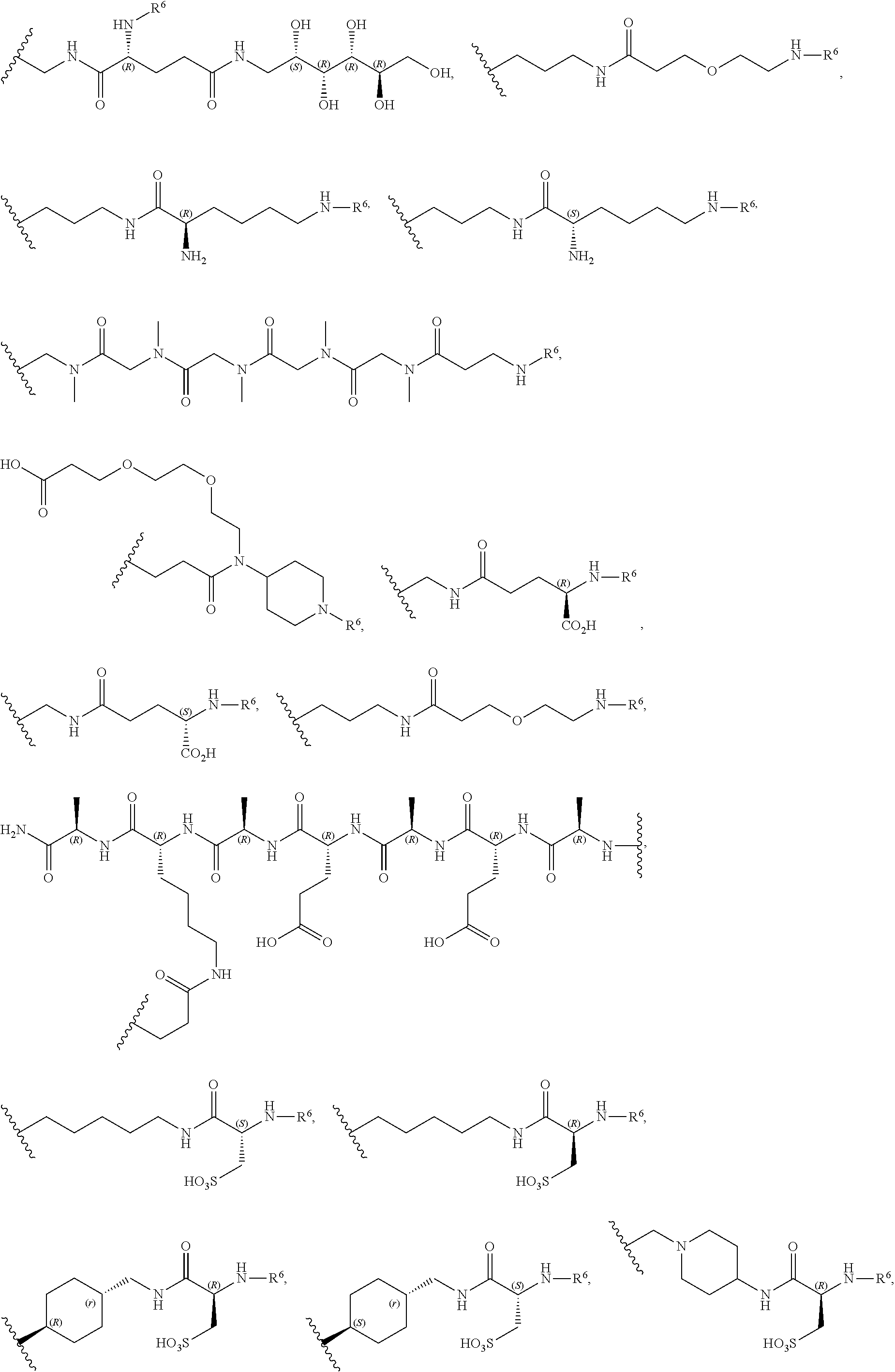

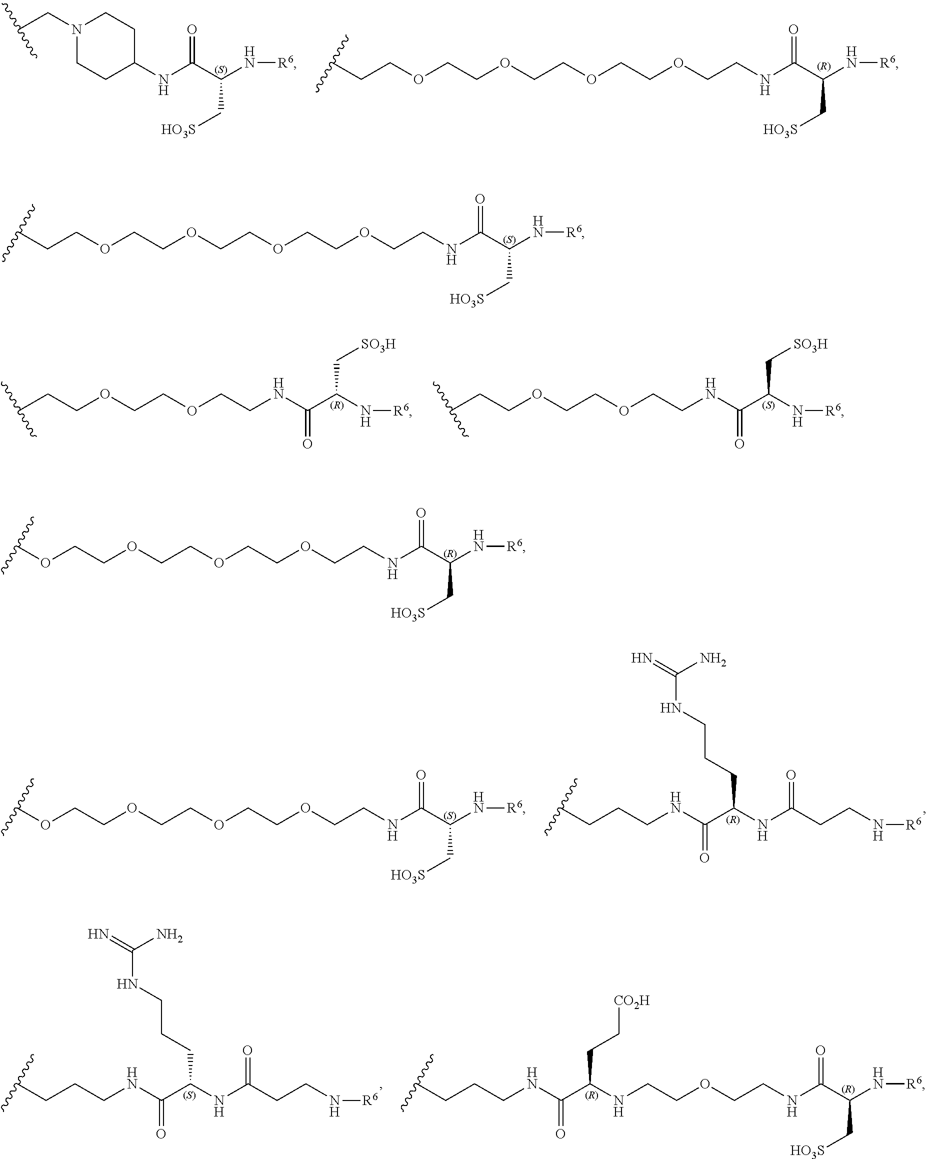

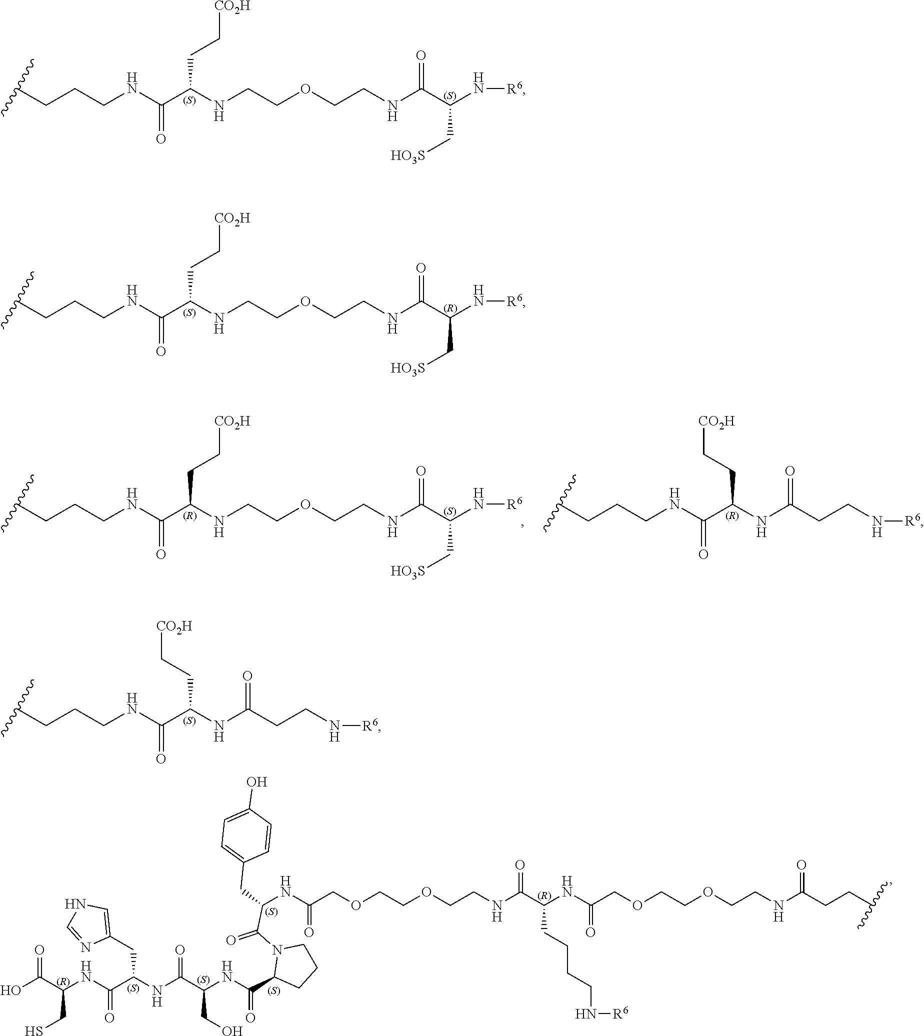

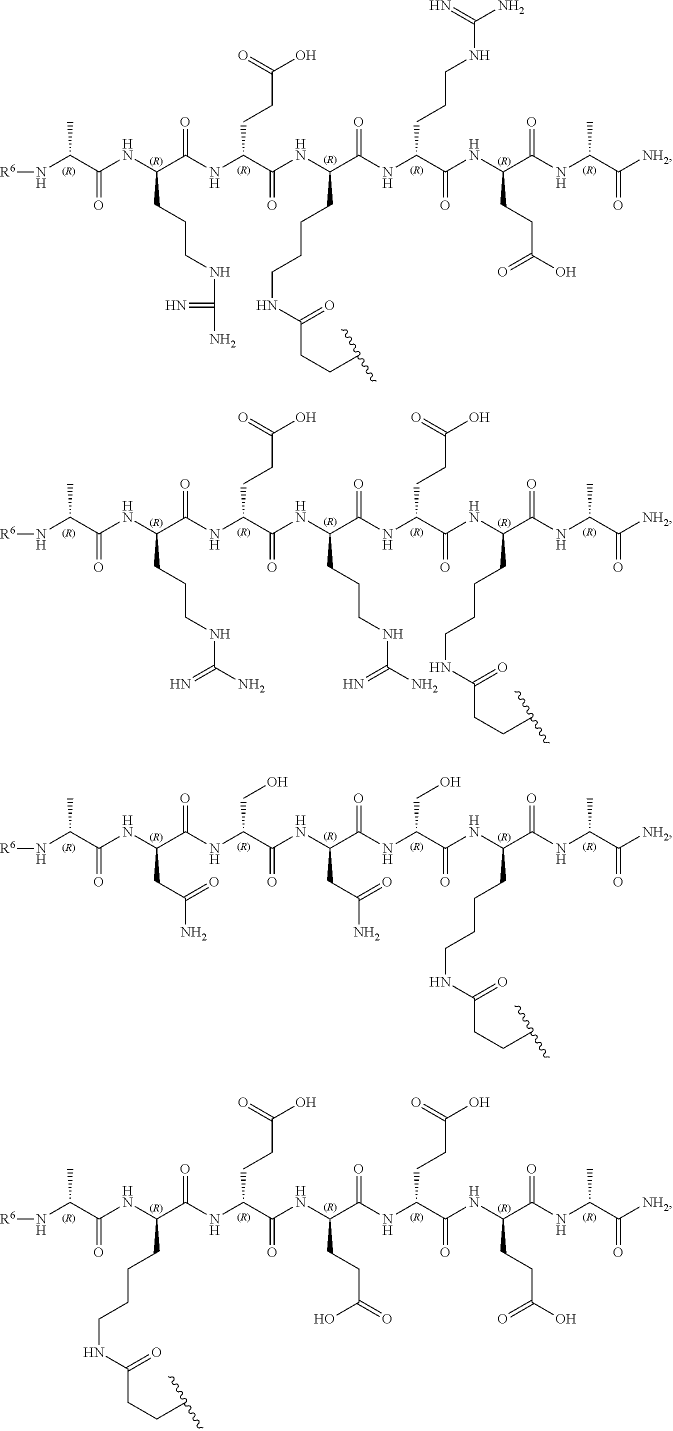

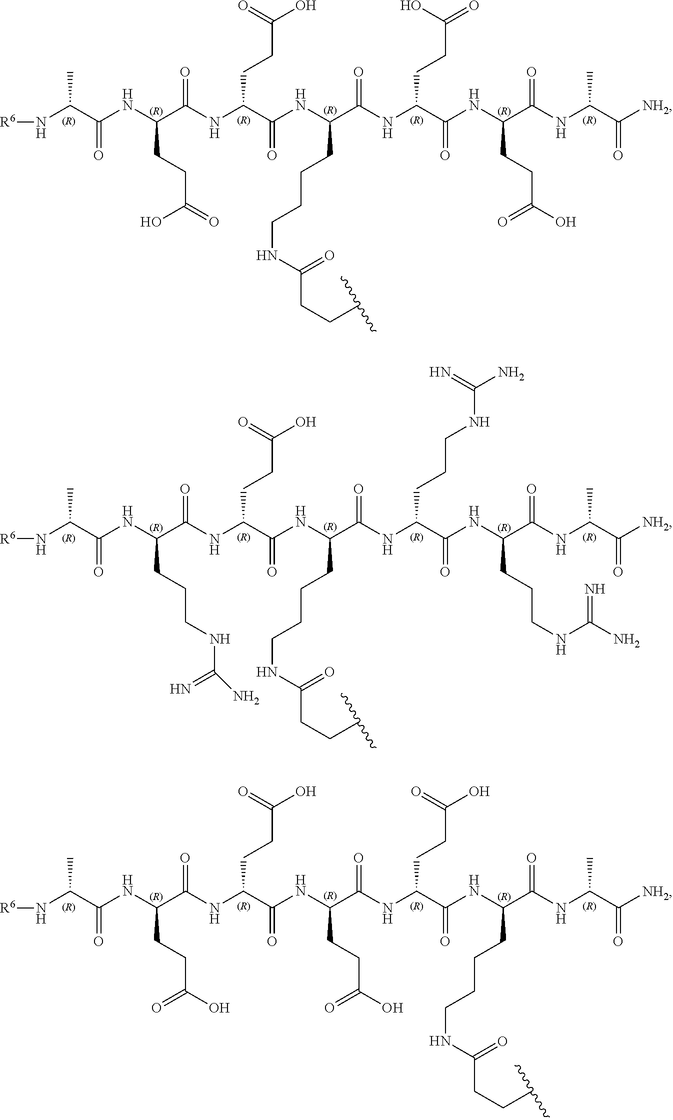

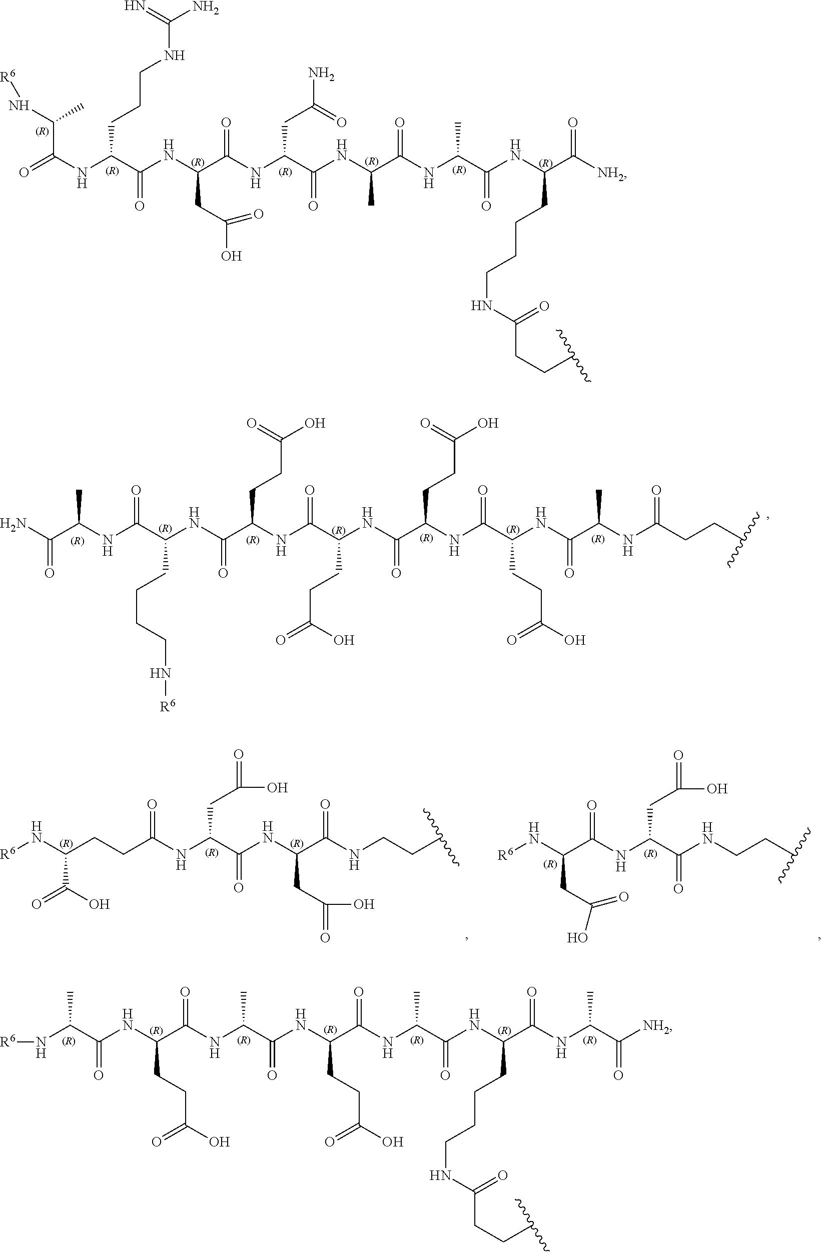

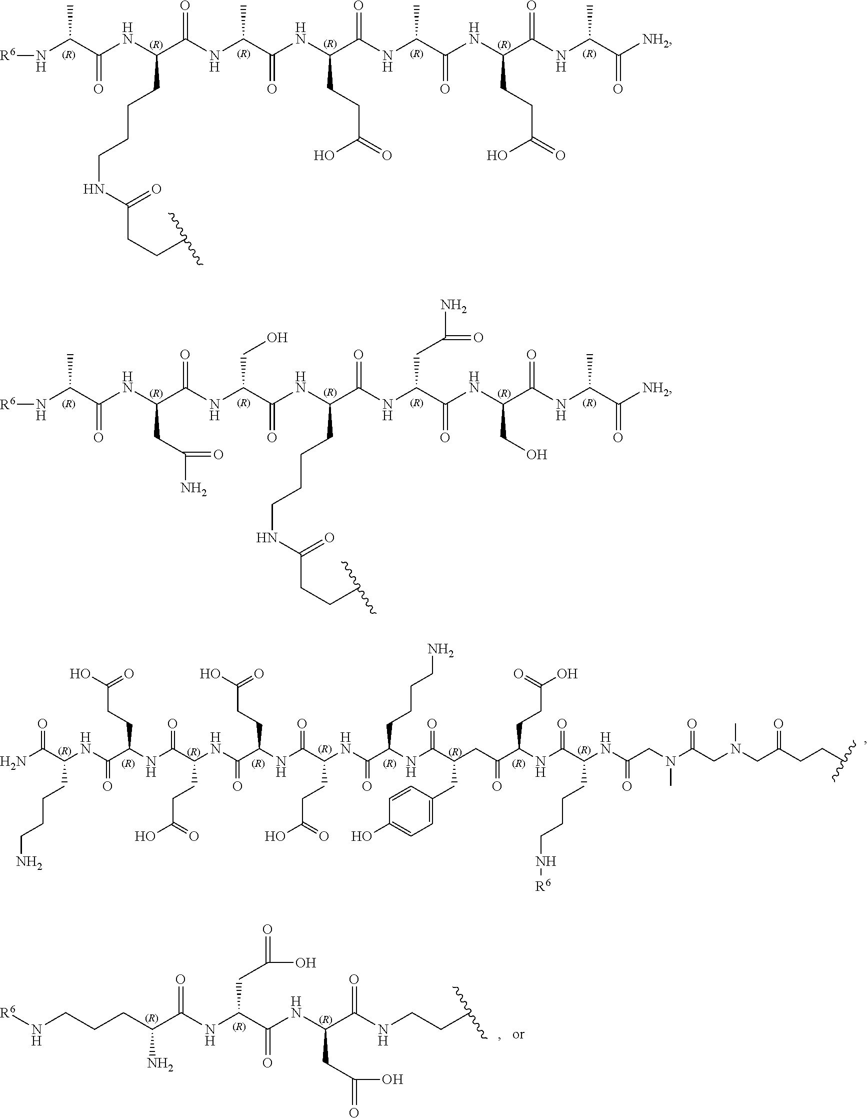

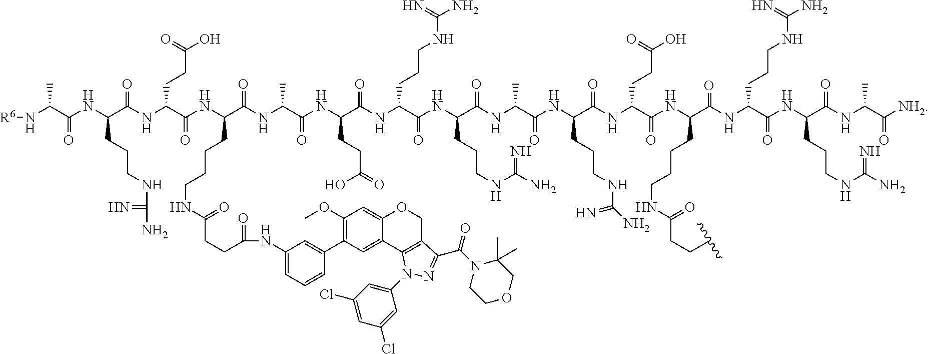

109. The compound of claim 10, or a pharmaceutically acceptable salt thereof, wherein R is:

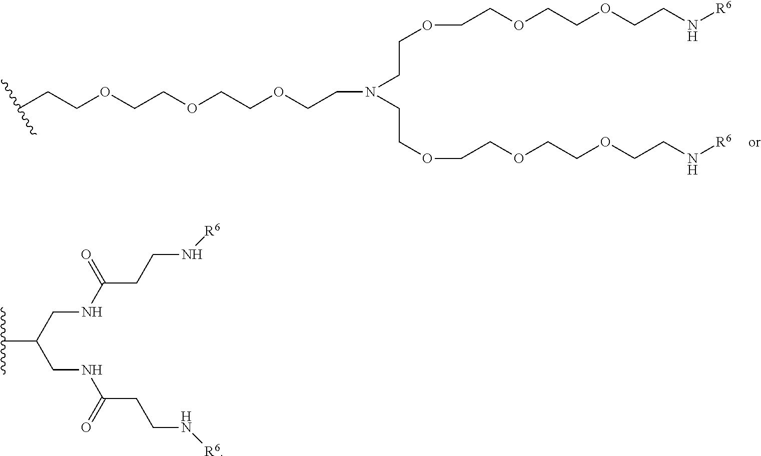

110. The compound of any one of claims 10-81, or a pharmaceutically acceptable salt thereof, wherein-L¹-R⁶ is: 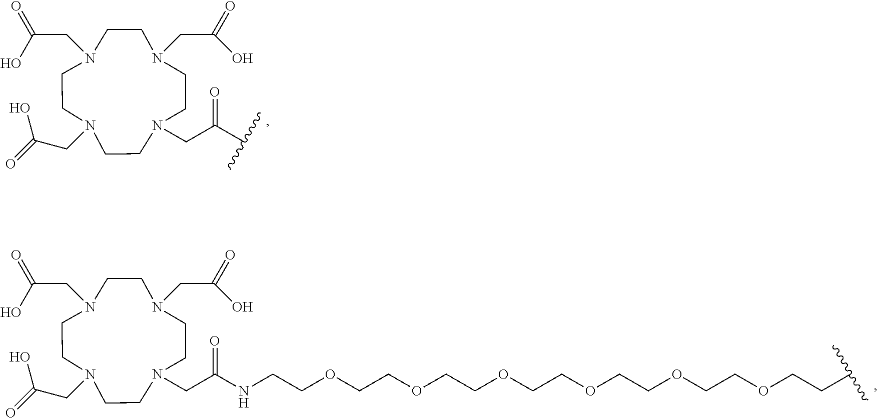 [SMILES: *C(=O)CN1CCN(CC(=O)O)CCN(CC(=O)O)CCN(CC(=O)O)CC1 ; *CCOCCOCCOCCOCCOCCOCCNC(=O)CN1CCN(CC(=O)O)CCN(CC(=O)O)CCN(CC(=O)O)CC1] 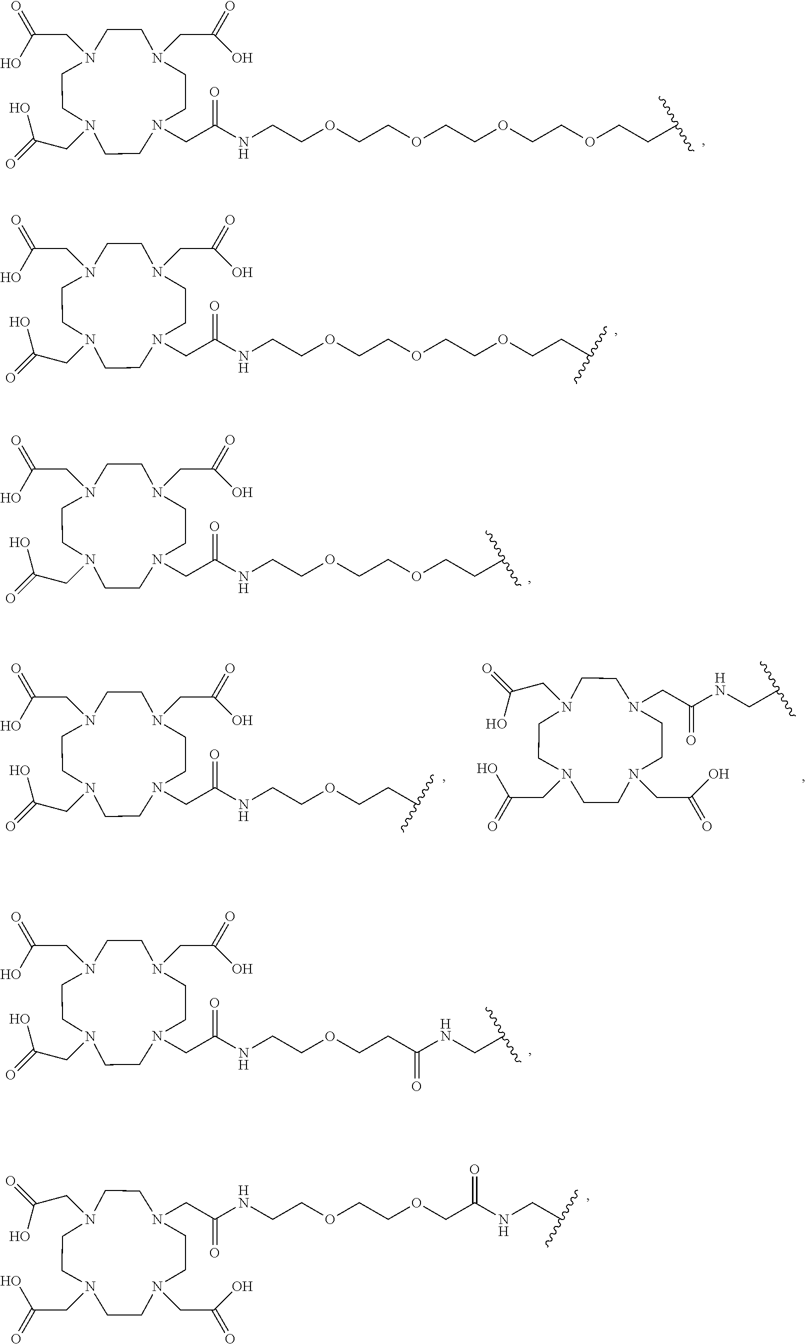 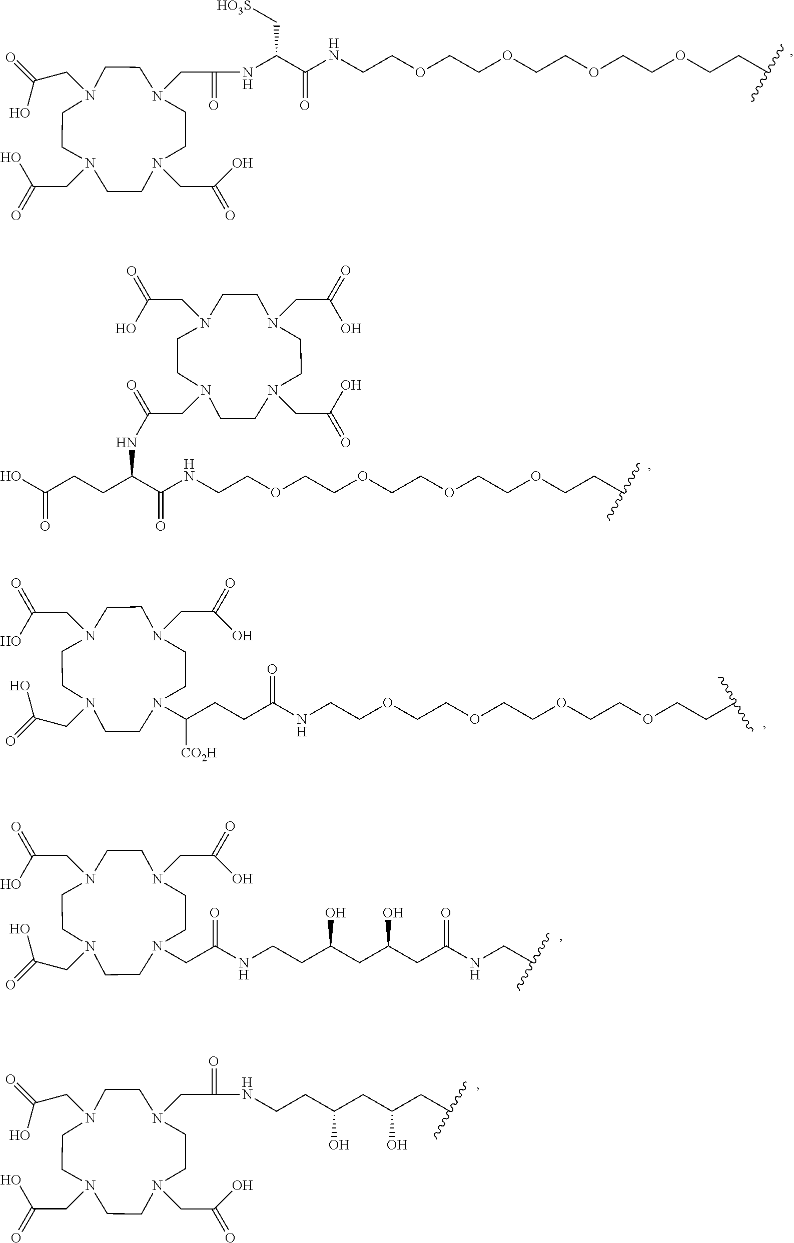 [SMILES: *CCOCCOCCOCCOCCNC(=O)[C@@H](CS(=O)(=O)O)NC(=O)CN1CCN(CC(=O)O)CCN(CC(=O)O)CCN(CC(=O)O)CC1 ; *CCOCCOCCOCCOCCNC(=O)[C@@H](CCC(=O)O)NC(=O)CN1CCN(CC(=O)O)CCN(CC(=O)O)CCN(CC(=O)O)CC1 ; *CCOCCOCCOCCOCCNC(=O)CCC(C(=O)O)N1CCN(CC(=O)O)CCN(CC(=O)O)CCN(CC(=O)O)CC1 ; *CNC(=O)C[C@H](O)C[C@H](O)CCNC(=O)CN1CCN(CC(=O)O)CCN(CC(=O)O)CCN(CC(=O)O)CC1 ; *C[C@H](O)C[C@H](O)CCNC(=O)CN1CCN(CC(=O)O)CCN(CC(=O)O)CCN(CC(=O)O)CC1] 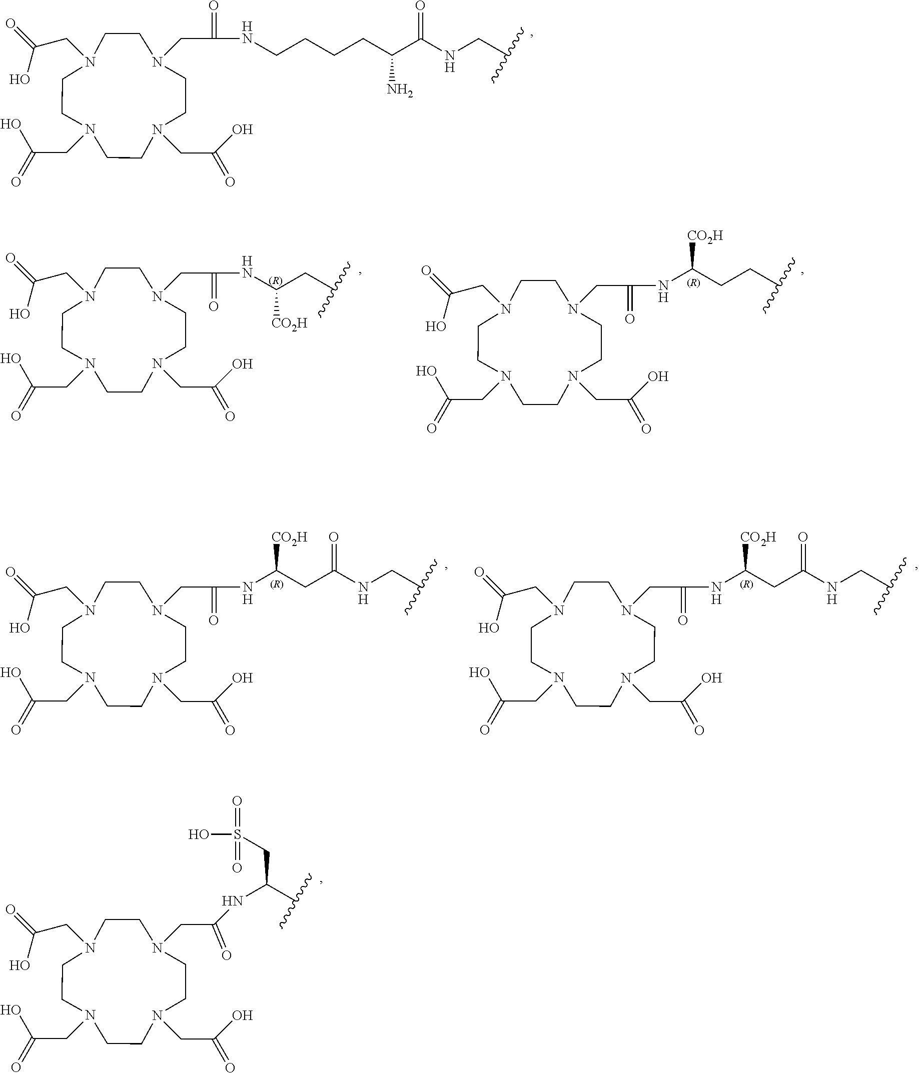 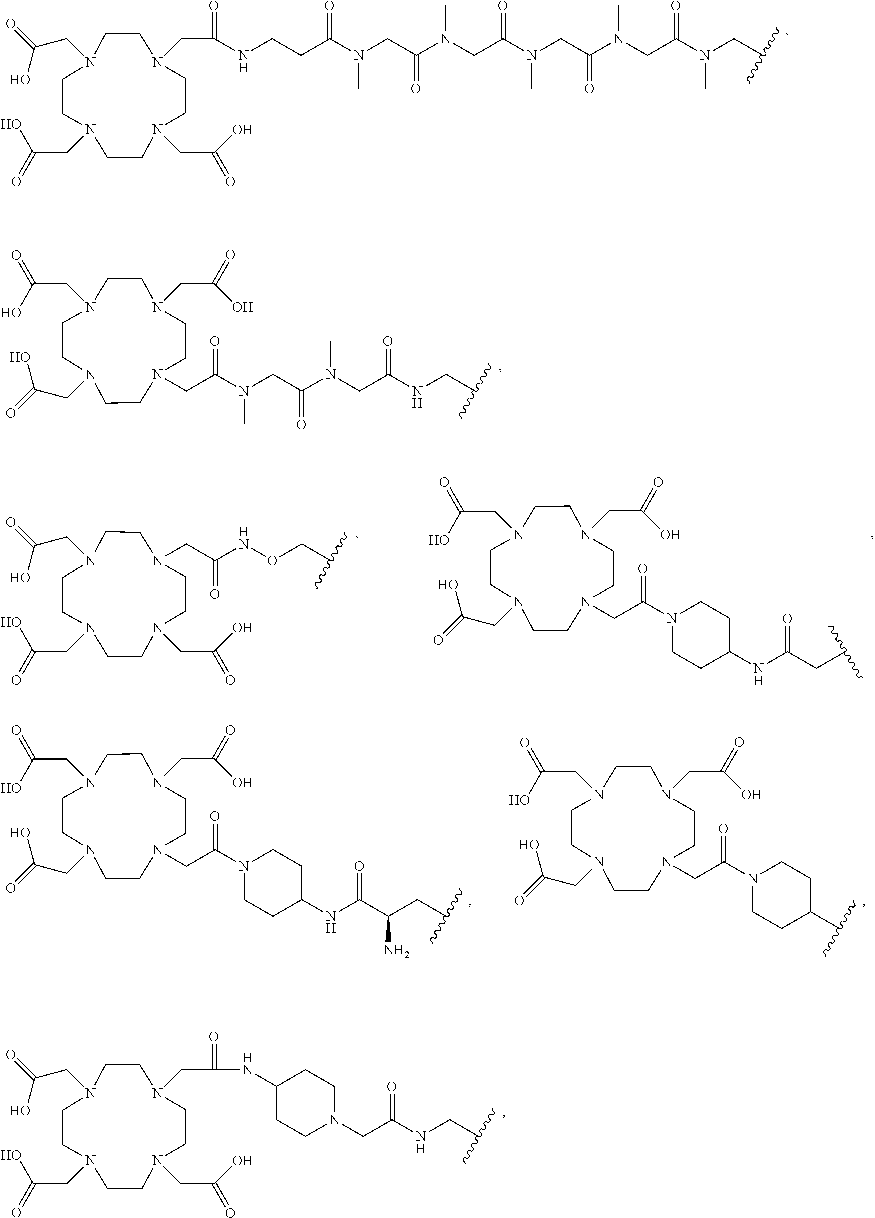 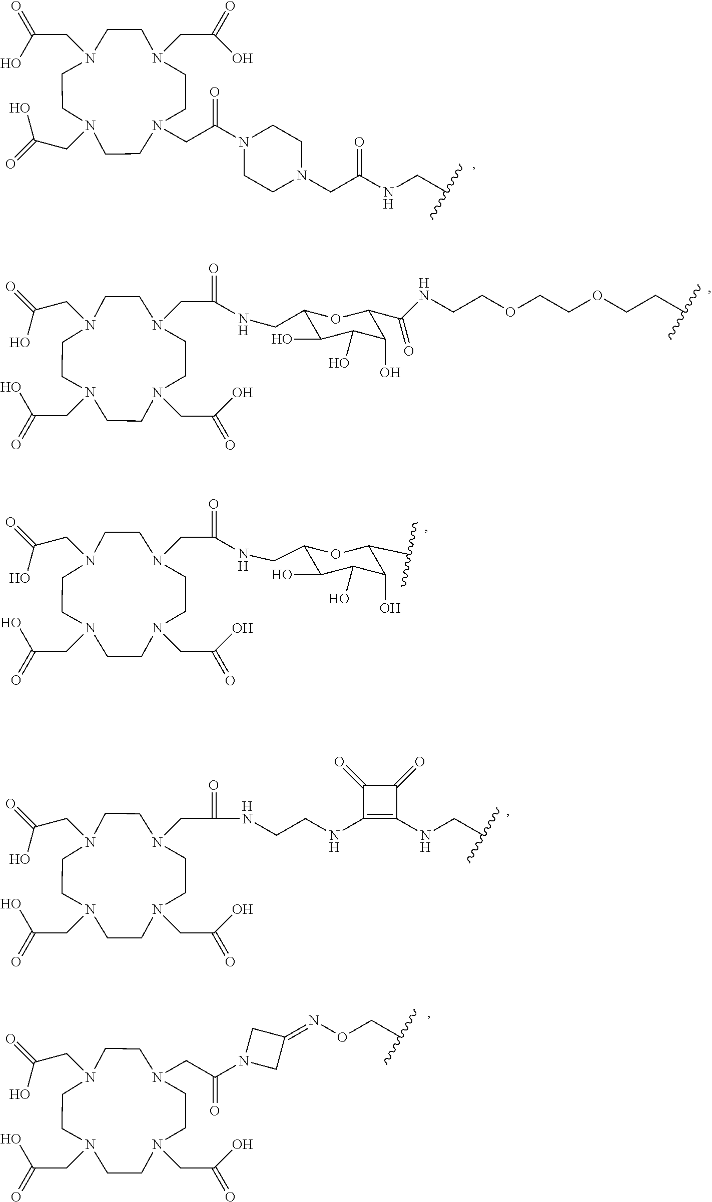 [SMILES: *CNC(=O)CN1CCN(C(=O)CN2CCN(CC(=O)O)CCN(CC(=O)O)CCN(CC(=O)O)CC2)CC1 ; *CCOCCOCCNC(=O)C1OC(CNC(=O)CN2CCN(CC(=O)O)CCN(CC(=O)O)CCN(CC(=O)O)CC2)C(O)C(O)C1O ; *C1OC(CNC(=O)CN2CCN(CC(=O)O)CCN(CC(=O)O)CCN(CC(=O)O)CC2)C(O)C(O)C1O ; *CNc1c(NCCNC(=O)CN2CCN(CC(=O)O)CCN(CC(=O)O)CCN(CC(=O)O)CC2)c(=O)c1=O ; *CON=C1CN(C(=O)CN2CCN(CC(=O)O)CCN(CC(=O)O)CCN(CC(=O)O)CC2)C1] 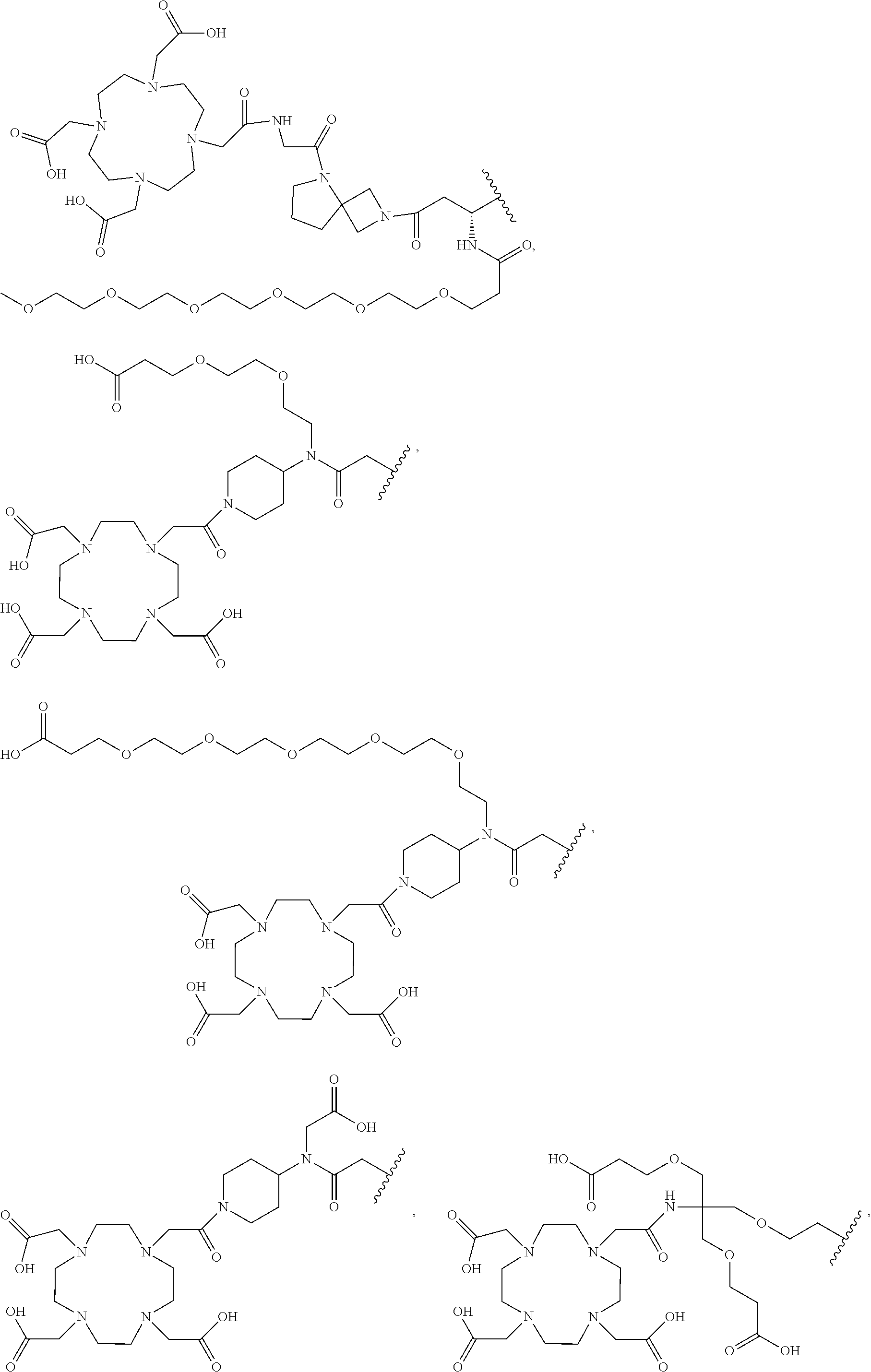 [SMILES: *[C@@H](CC(=O)N1CC2(CCCN2C(=O)CNC(=O)CN2CCN(CC(=O)O)CCN(CC(=O)O)CCN(CC(=O)O)CC2)C1)NC(=O)CCOCCOCCOCCOCCOCCOC ; *CC(=O)N(CCOCCOCCC(=O)O)C1CCN(C(=O)CN2CCN(CC(=O)O)CCN(CC(=O)O)CCN(CC(=O)O)CC2)CC1 ; *CC(=O)N(CCOCCOCCOCCOCCOCCC(=O)O)C1CCN(C(=O)CN2CCN(CC(=O)O)CCN(CC(=O)O)CCN(CC(=O)O)CC2)CC1 ; *CC(=O)N(CC(=O)O)C1CCN(C(=O)CN2CCN(CC(=O)O)CCN(CC(=O)O)CCN(CC(=O)O)CC2)CC1 ; *CCOCC(COCCC(=O)O)(COCCC(=O)O)NC(=O)CN1CCN(CC(=O)O)CCN(CC(=O)O)CCN(CC(=O)O)CC1] 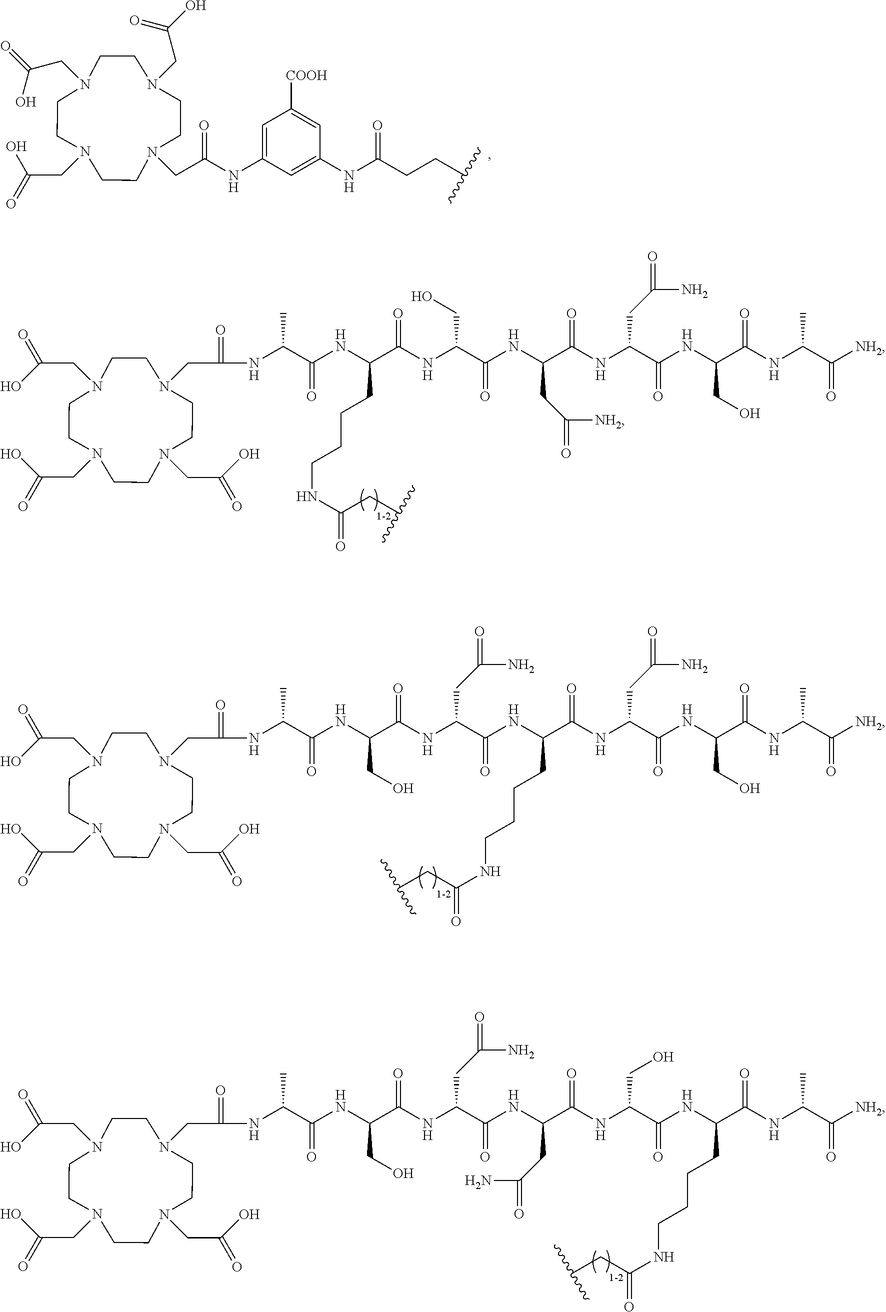 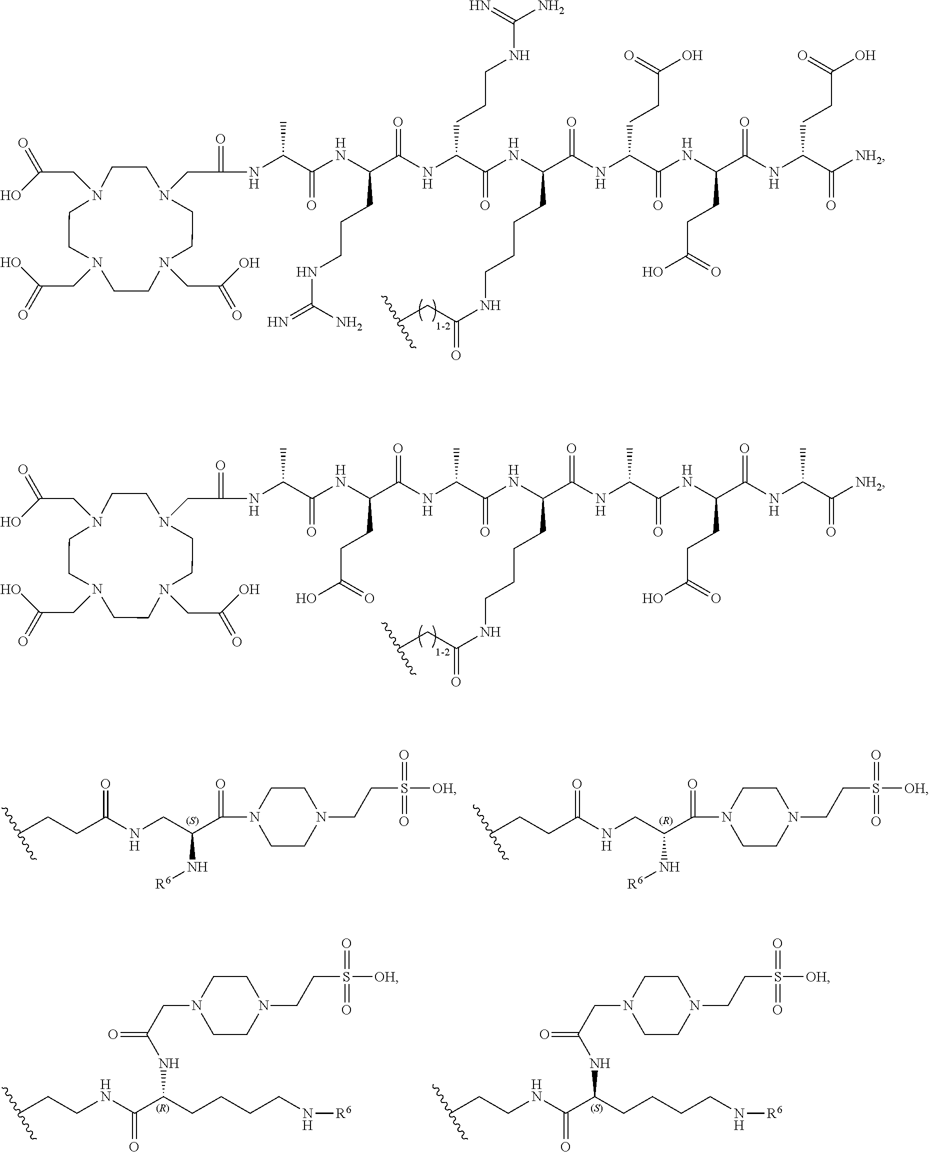 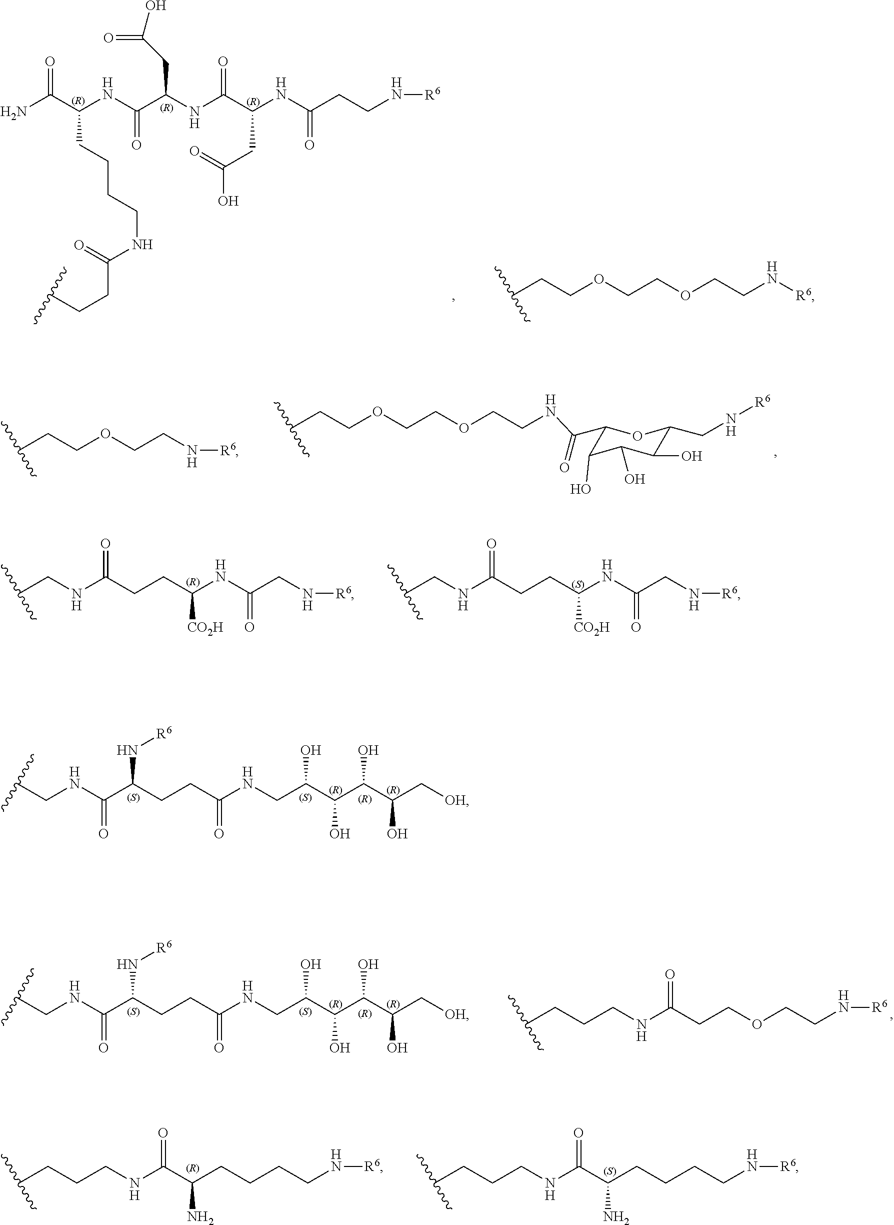 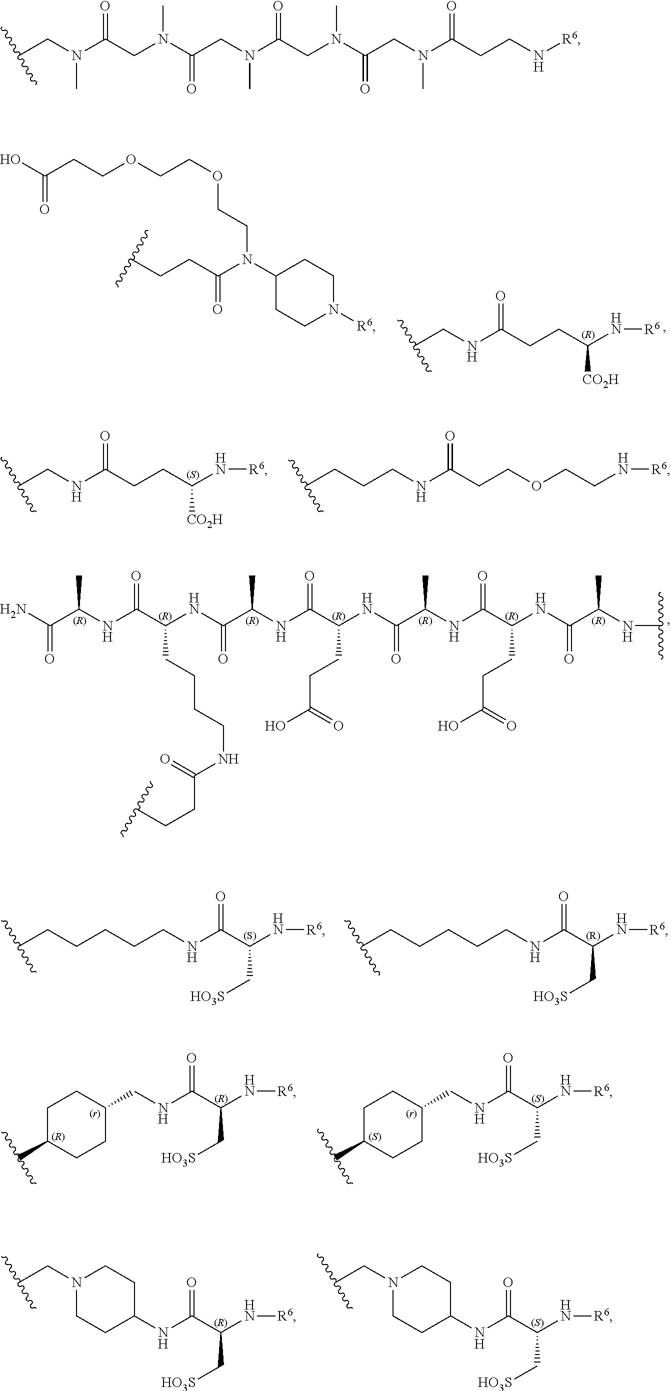 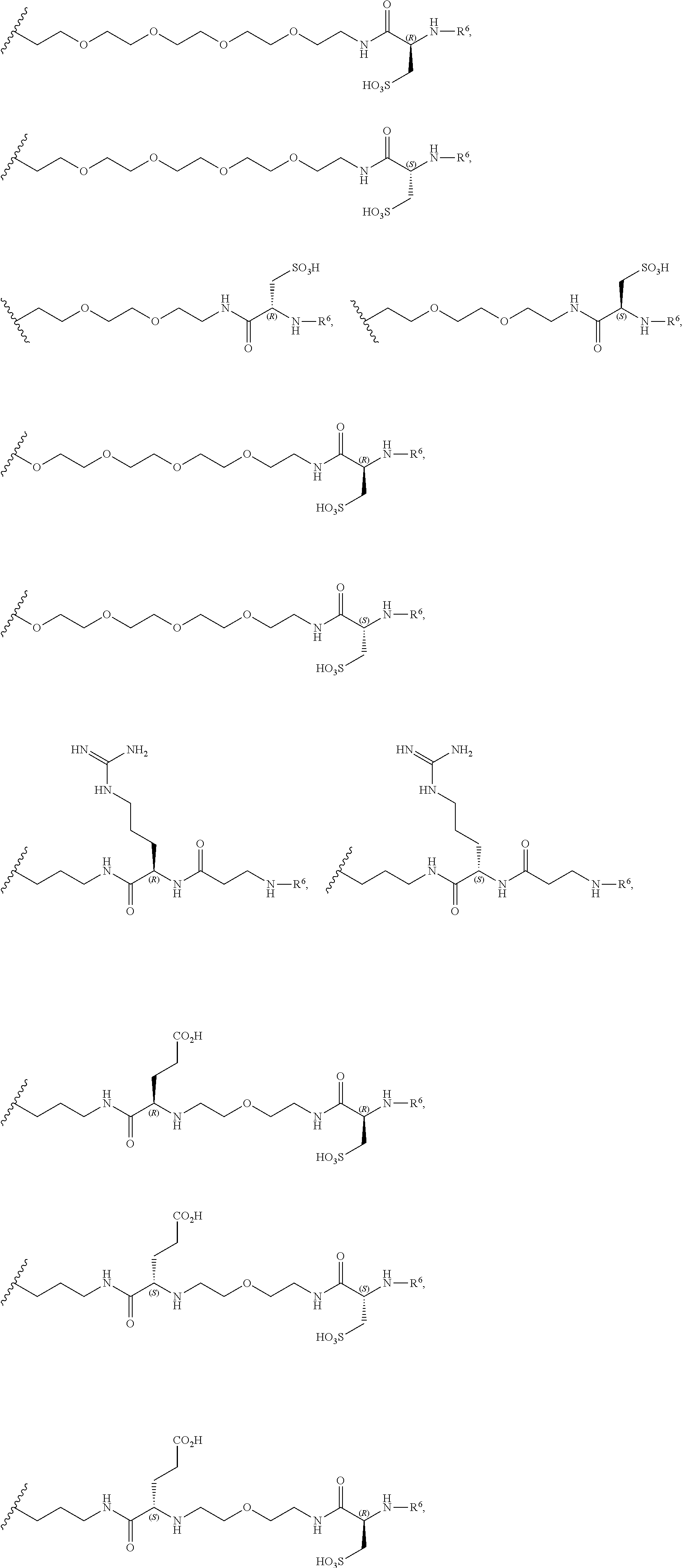 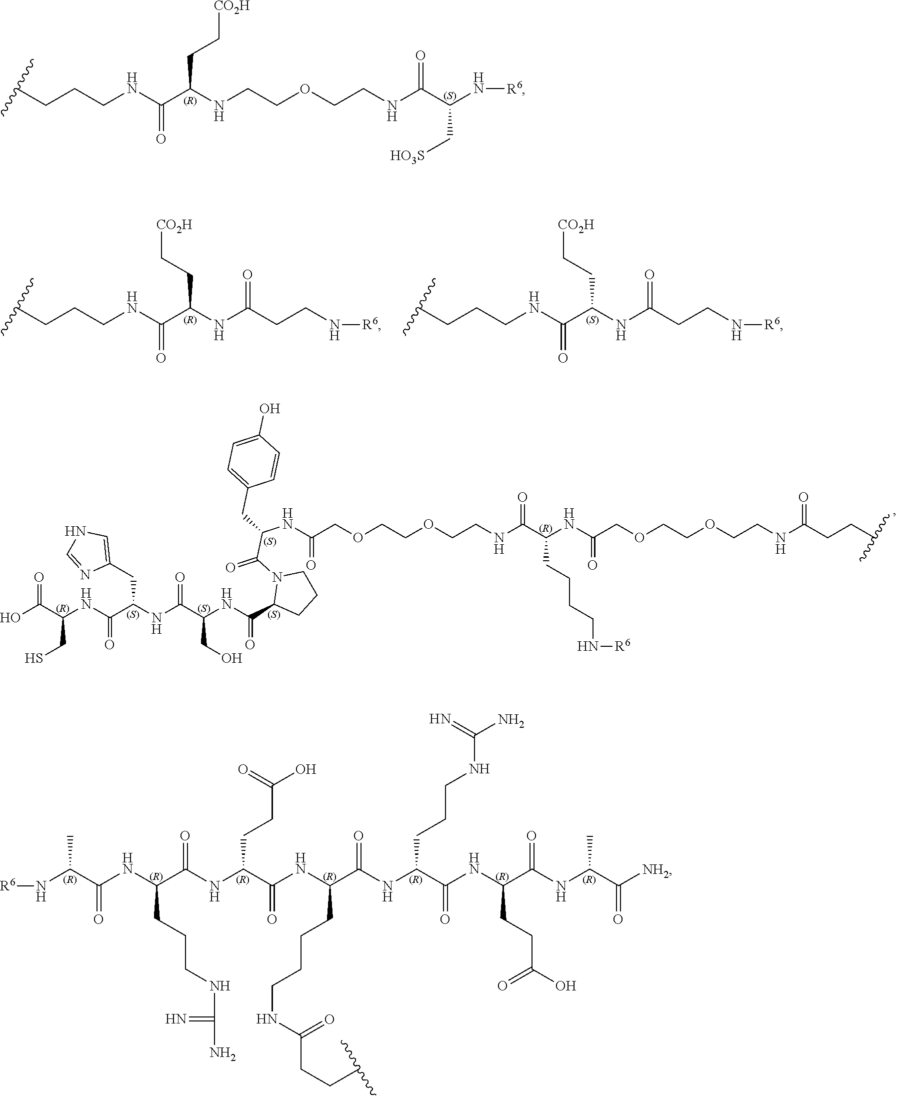 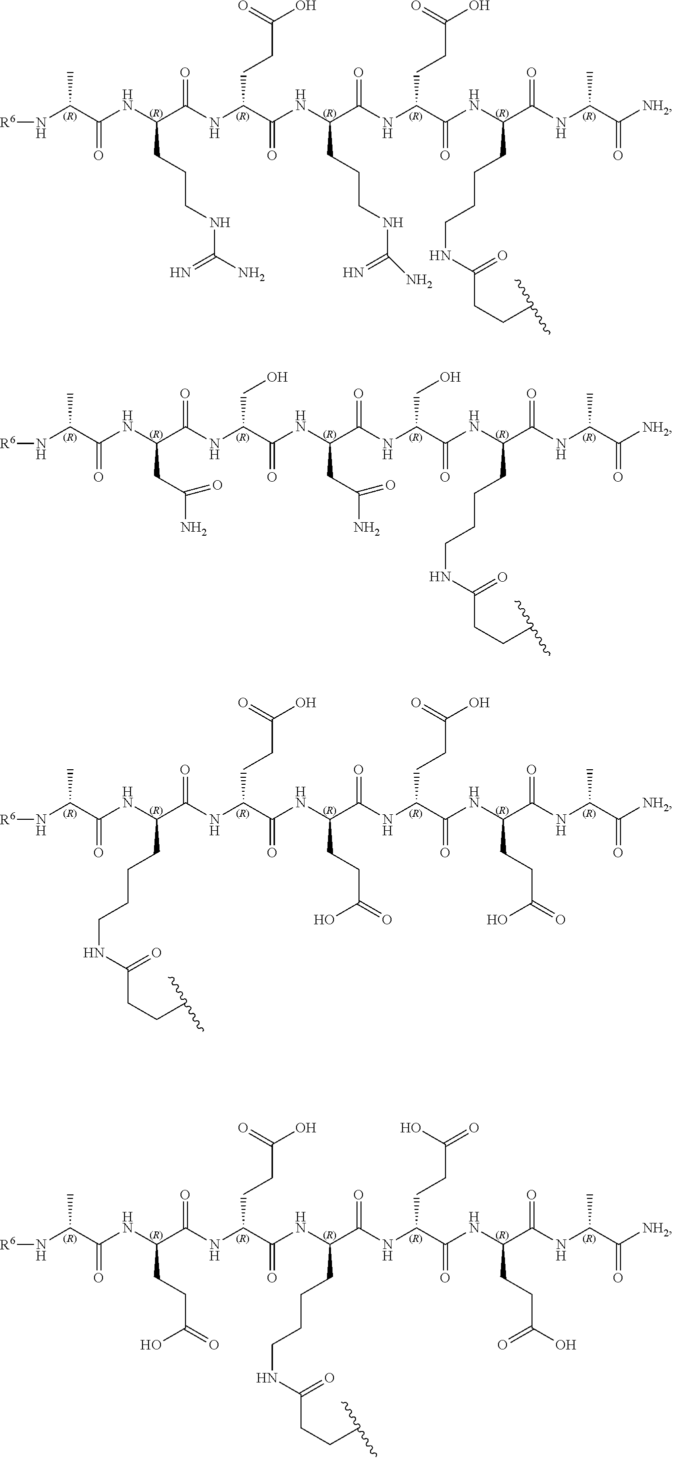 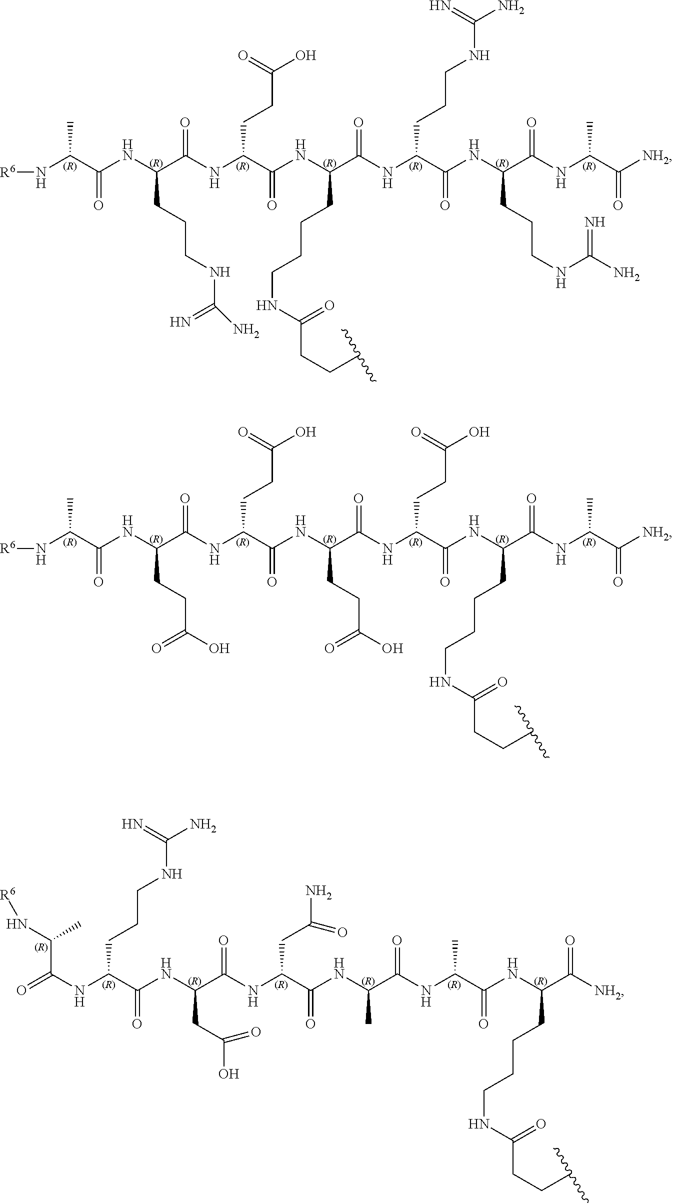 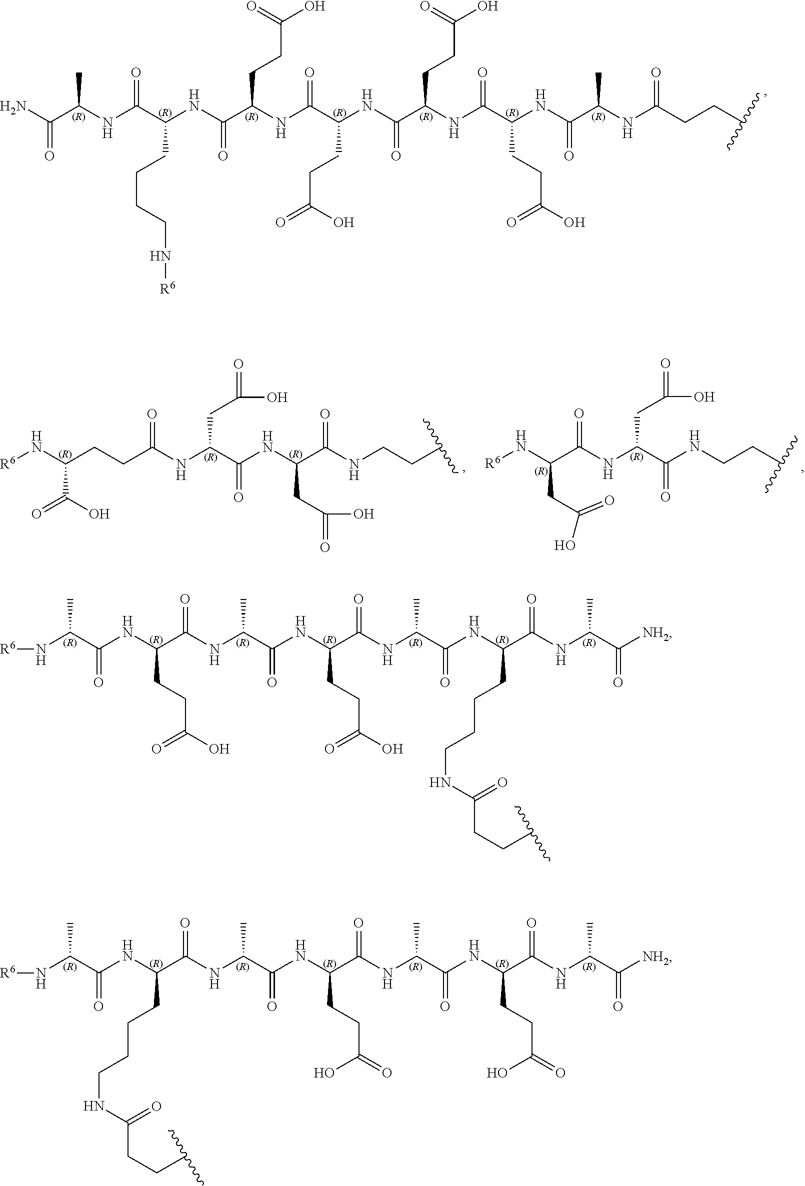 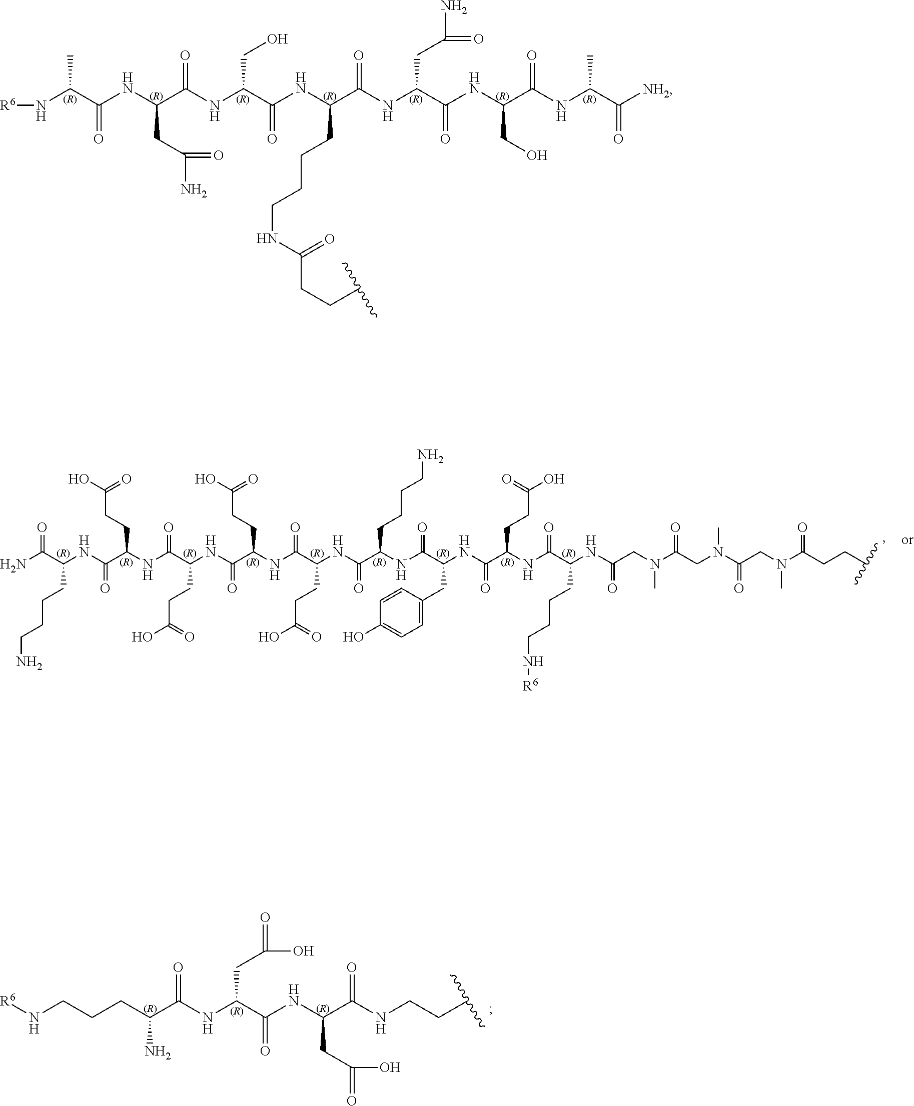
wherein R⁶ is

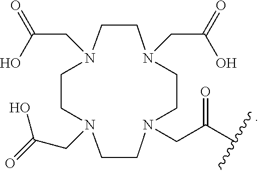 [SMILES: *C(=O)CN1CCN(CC(=O)O)CCN(CC(=O)O)CCN(CC(=O)O)CC1]

111. The compound of claim 10, or a pharmaceutically acceptable salt thereof, wherein R is:

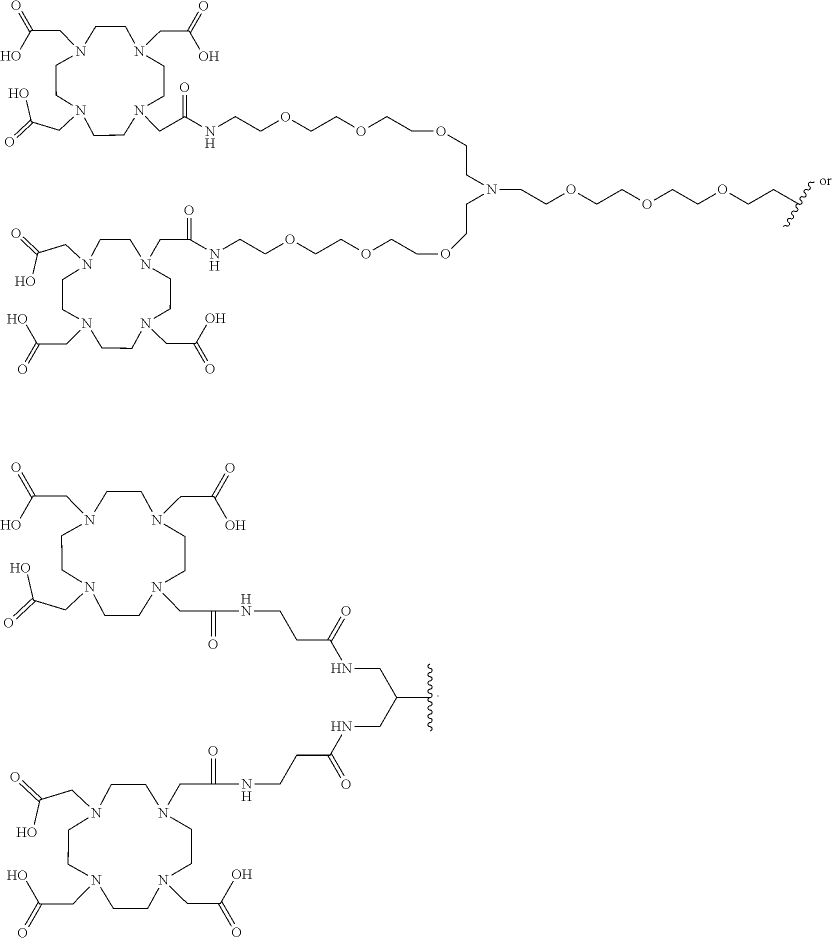 [SMILES: *CCOCCOCCOCCN(CCOCCOCCOCCNC(=O)CN1CCN(CC(=O)O)CCN(CC(=O)O)CCN(CC(=O)O)CC1)CCOCCOCCOCCNC(=O)CN1CCN(CC(=O)O)CCN(CC(=O)O)CCN(CC(=O)O)CC1 ; *C(CNC(=O)CCNC(=O)CN1CCN(CC(=O)O)CCN(CC(=O)O)CCN(CC(=O)O)CC1)CNC(=O)CCNC(=O)CN1CCN(CC(=O)O)CCN(CC(=O)O)CCN(CC(=O)O)CC1]

112. The compound of claim 11, or a pharmaceutically acceptable salt thereof, wherein the compound of Formula (I) has one of the following structures, or a pharmaceutically acceptable salt thereof. 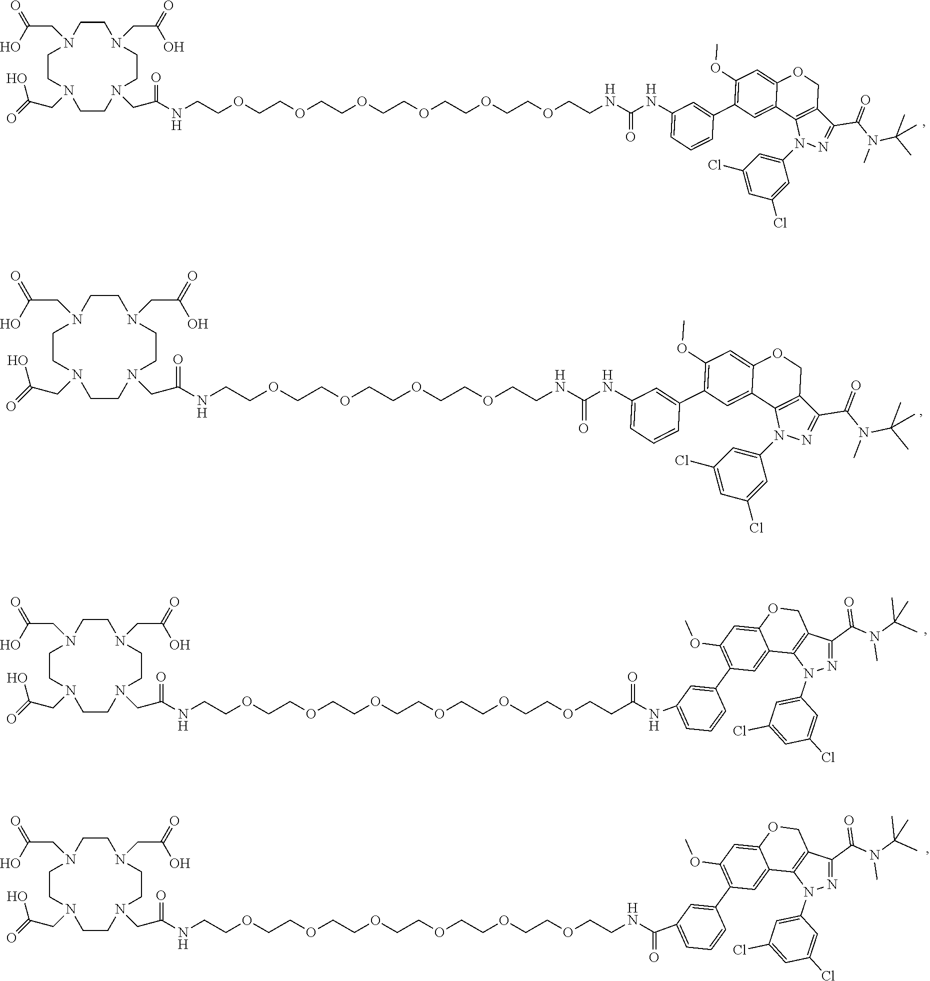 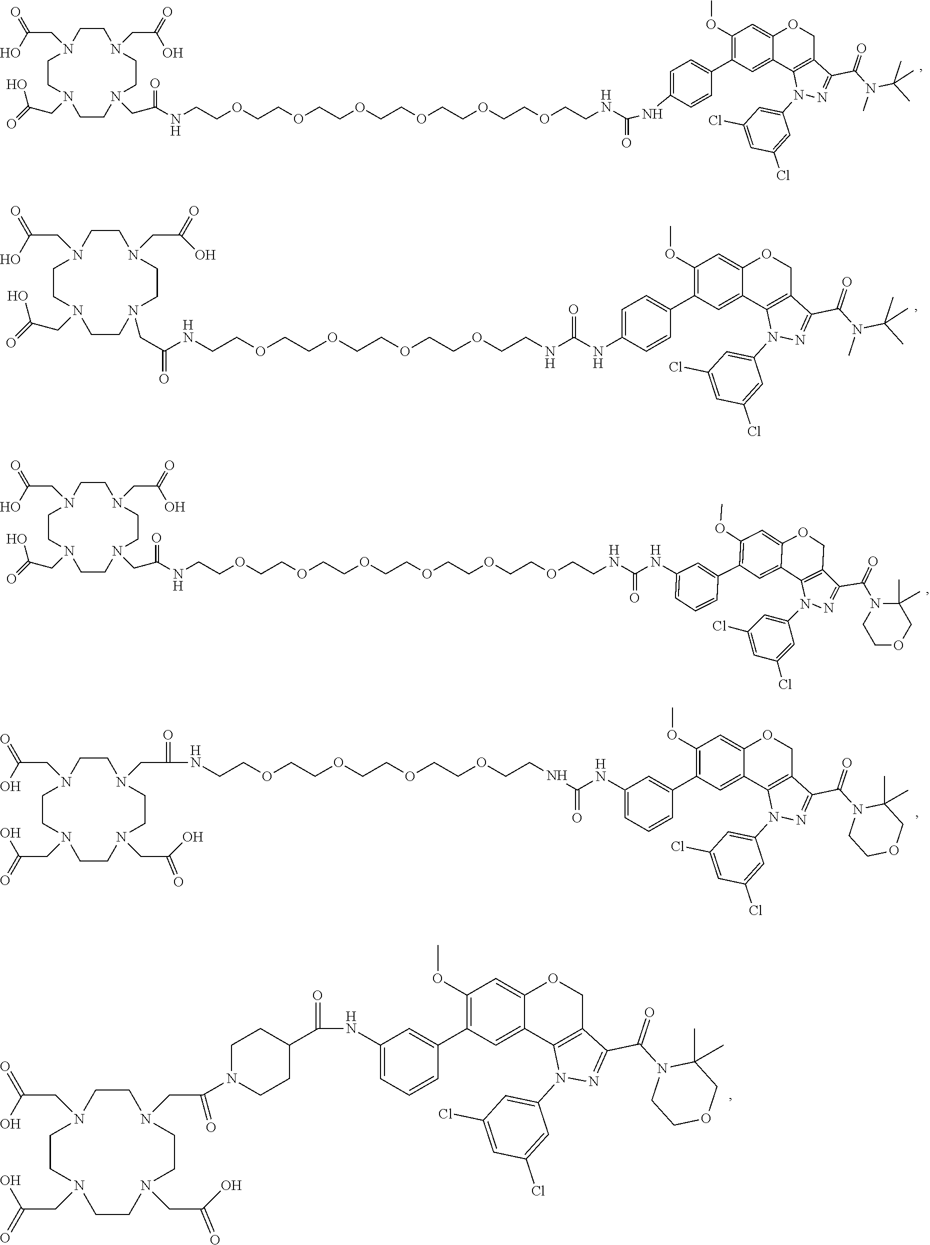 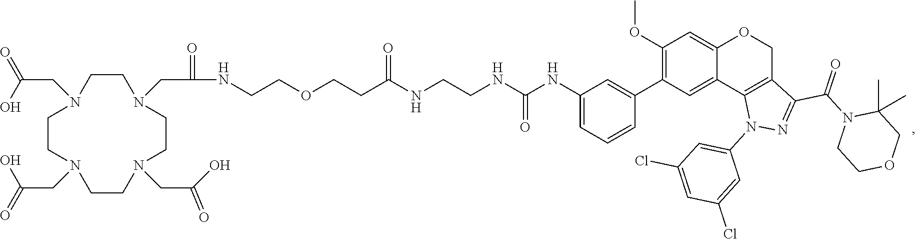 [SMILES: COc1cc2c(cc1-c1cccc(NC(=O)NCCNC(=O)CCOCCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2] 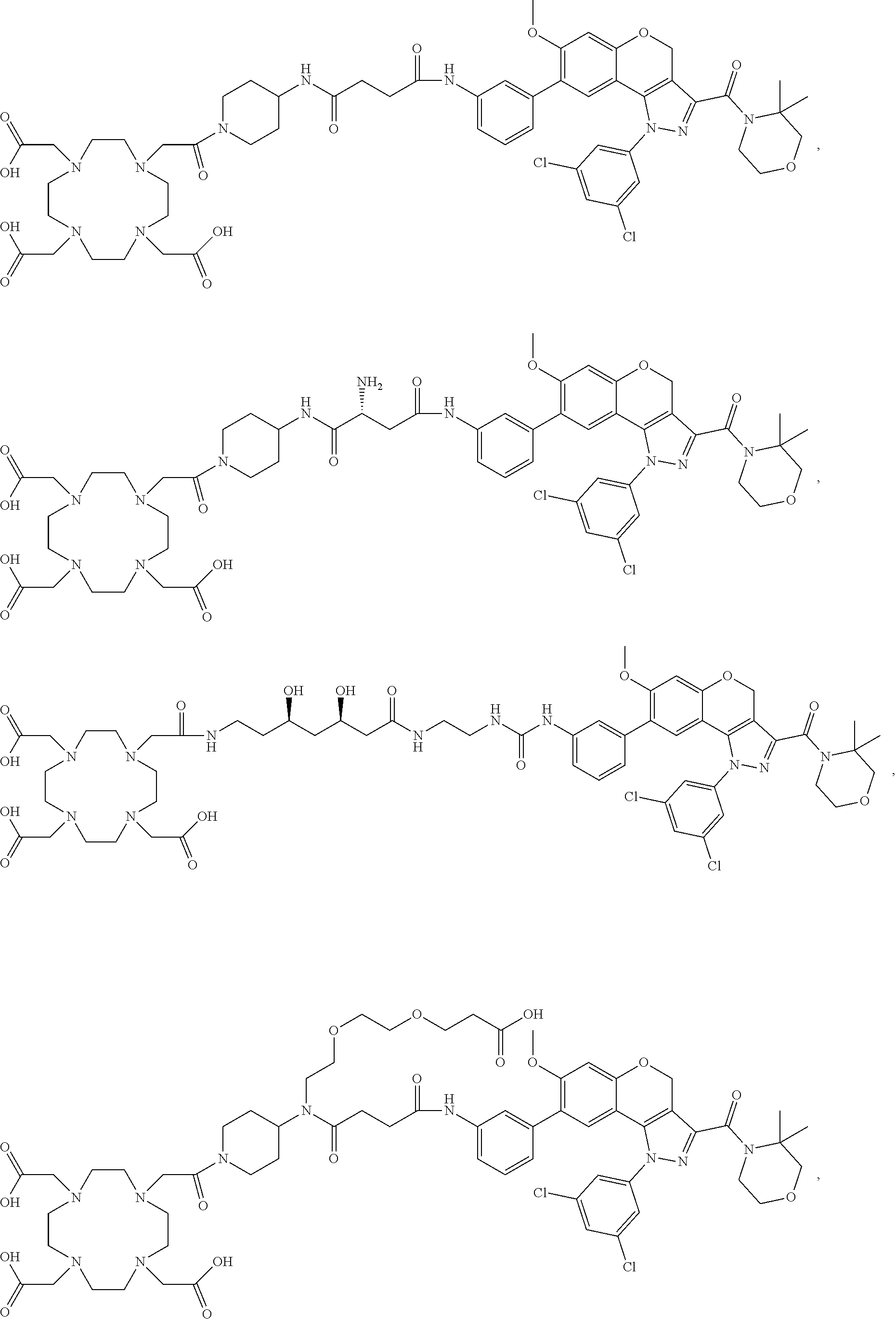 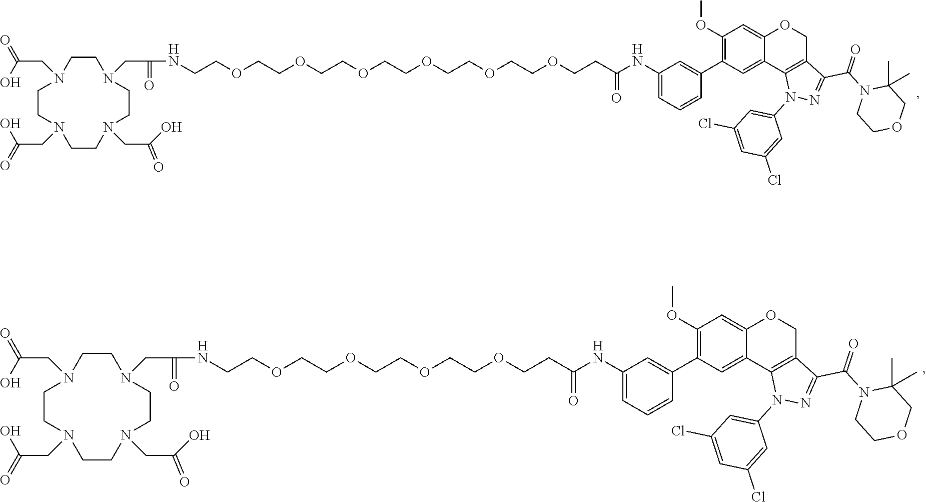 [SMILES: COc1cc2c(cc1-c1cccc(NC(=O)CCOCCOCCOCCOCCOCCOCCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2 ; COc1cc2c(cc1-c1cccc(NC(=O)CCOCCOCCOCCOCCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2] 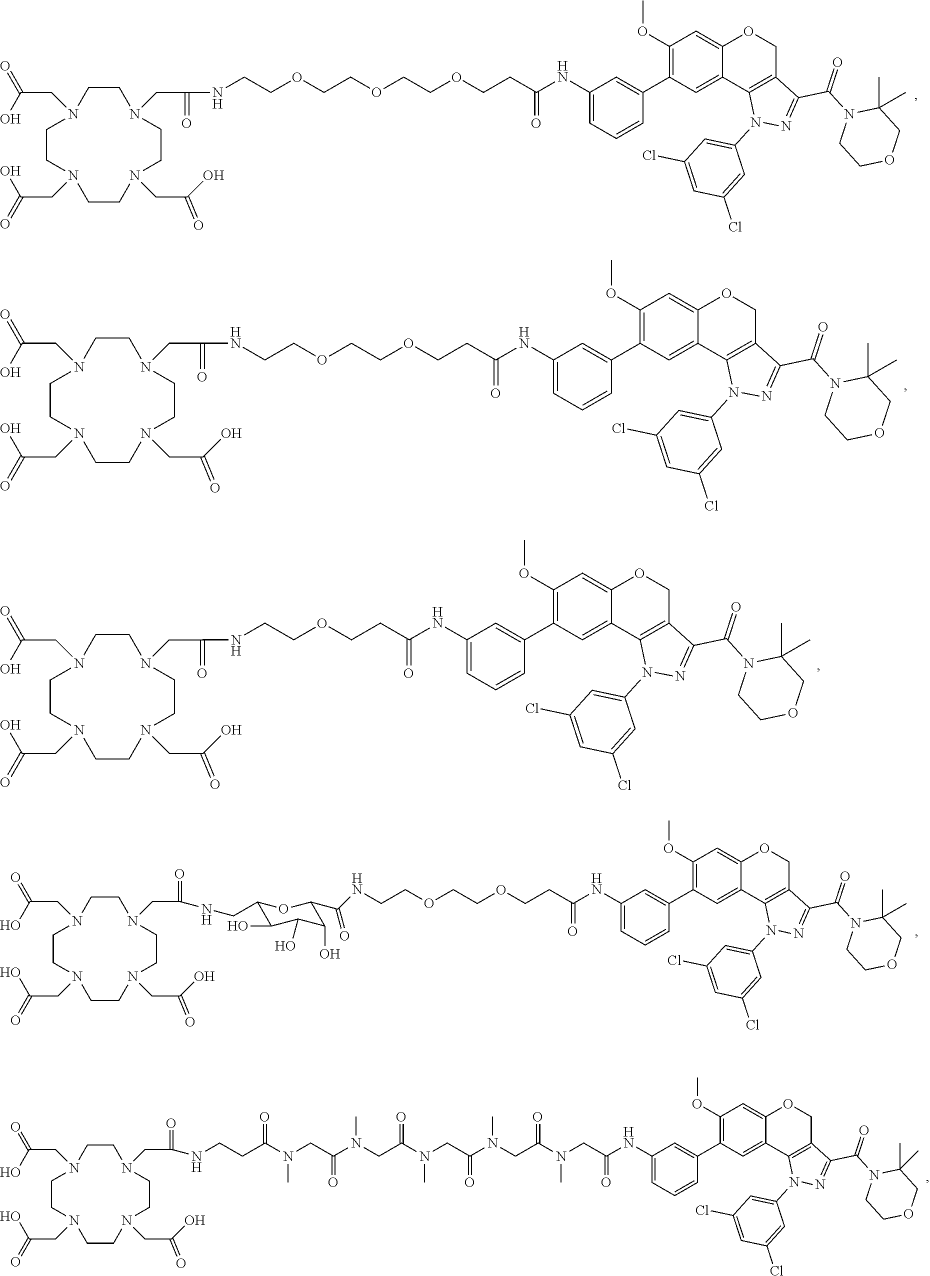 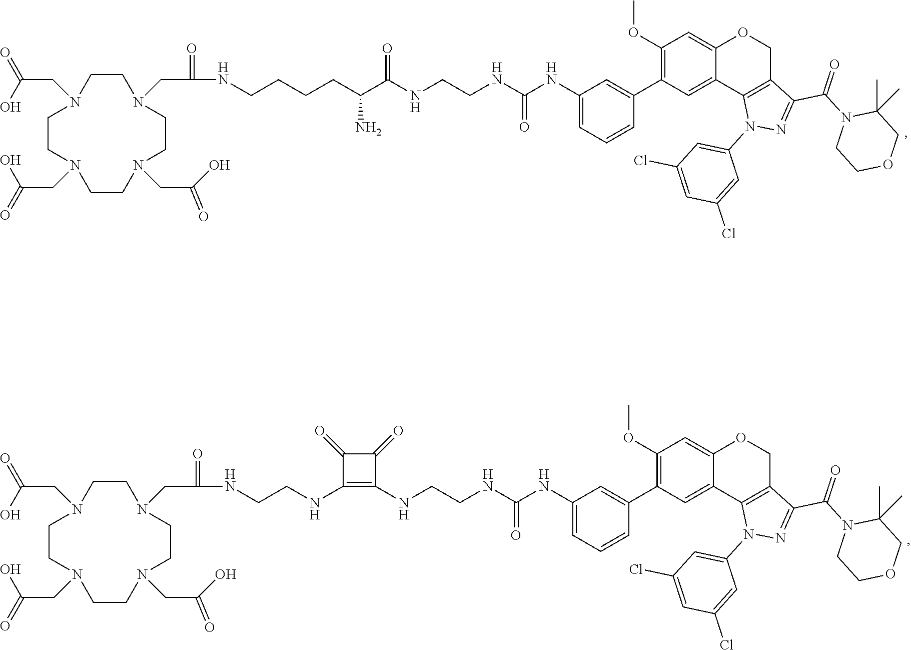 [SMILES: COc1cc2c(cc1-c1cccc(NC(=O)NCCNC(=O)[C@H](N)CCCCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2 ; COc1cc2c(cc1-c1cccc(NC(=O)NCCNc3c(NCCNC(=O)CN4CCN(CC(=O)O)CCN(CC(=O)O)CCN(CC(=O)O)CC4)c(=O)c3=O)c1)-c1c(c(C(=O)N3CCOCC3(C)C)nn1-c1cc(Cl)cc(Cl)c1)CO2] 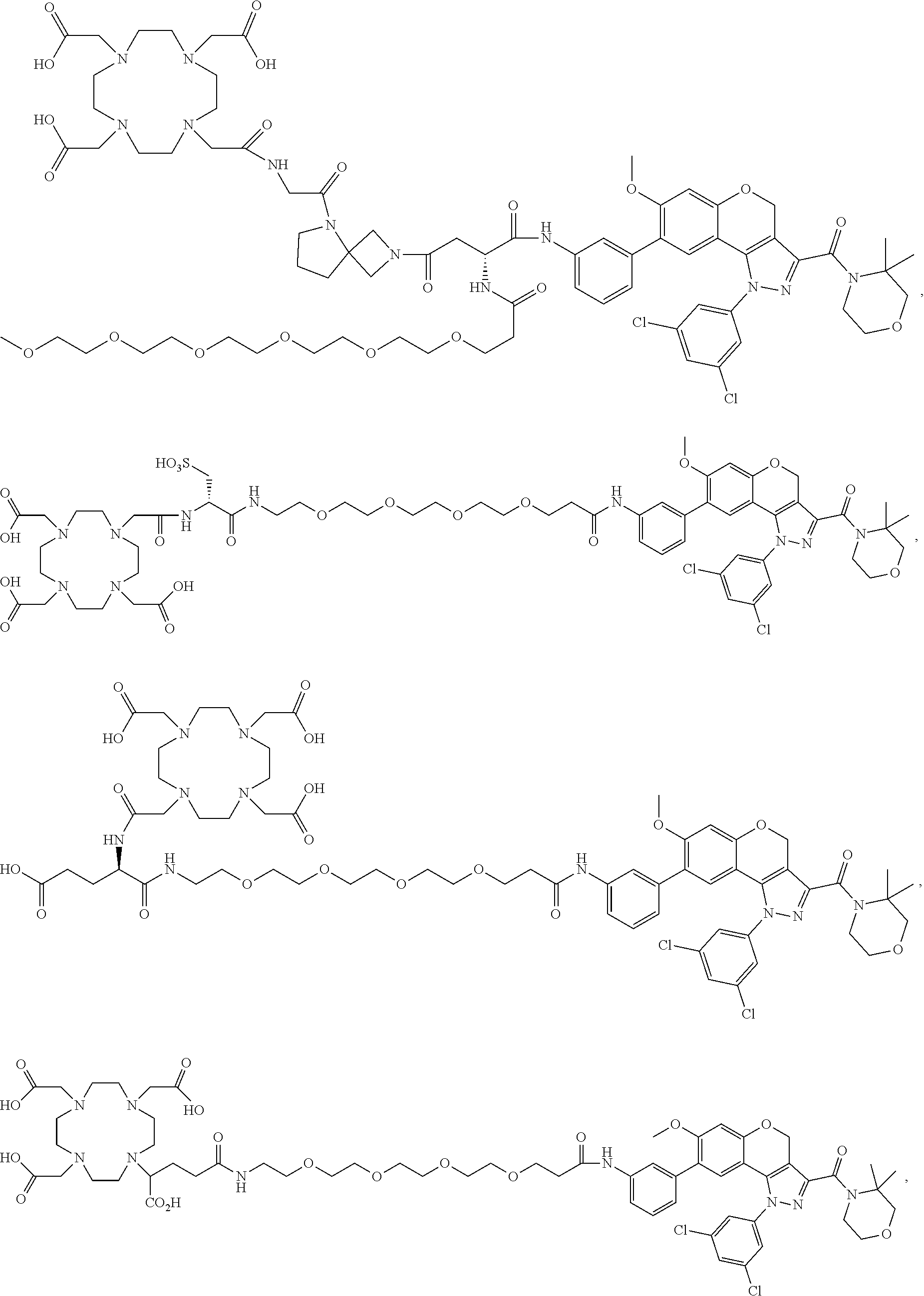 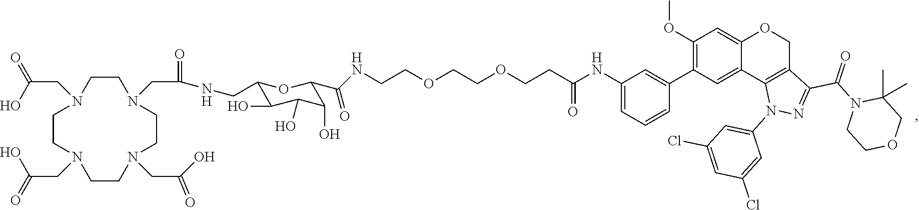 [SMILES: COc1cc2c(cc1-c1cccc(NC(=O)CCOCCOCCNC(=O)C3OC(CNC(=O)CN4CCN(CC(=O)O)CCN(CC(=O)O)CCN(CC(=O)O)CC4)C(O)C(O)C3O)c1)-c1c(c(C(=O)N3CCOCC3(C)C)nn1-c1cc(Cl)cc(Cl)c1)CO2] 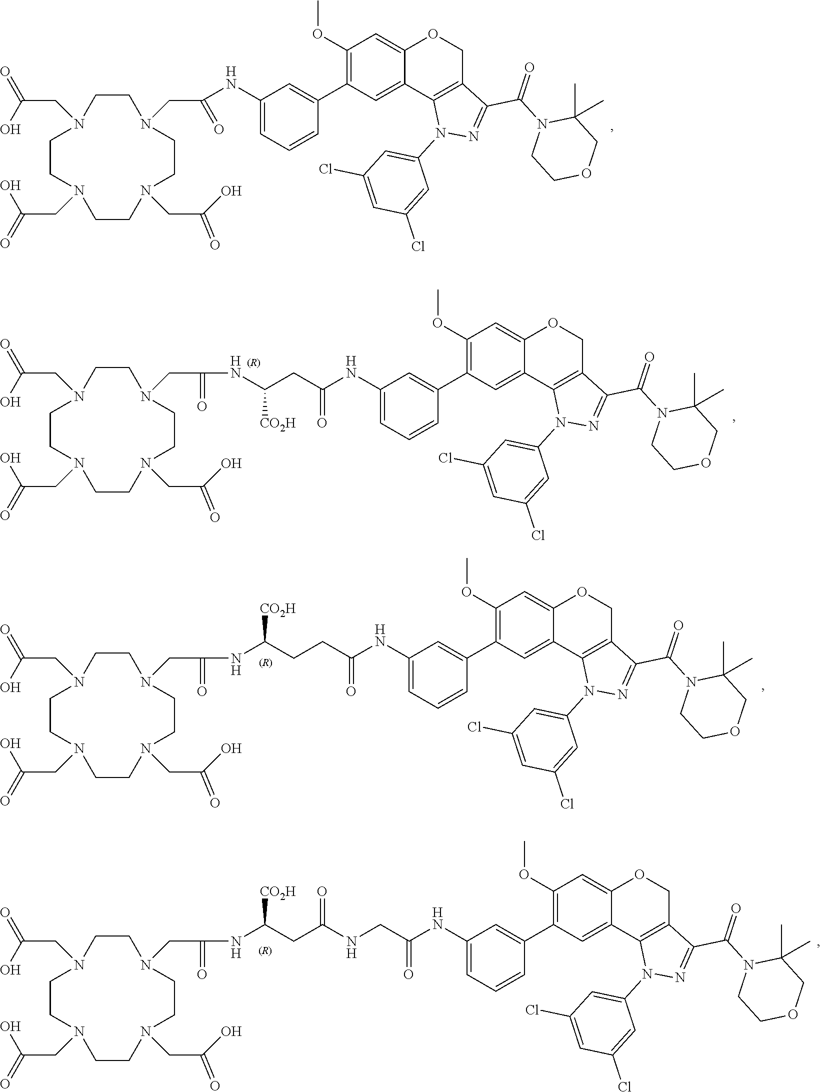 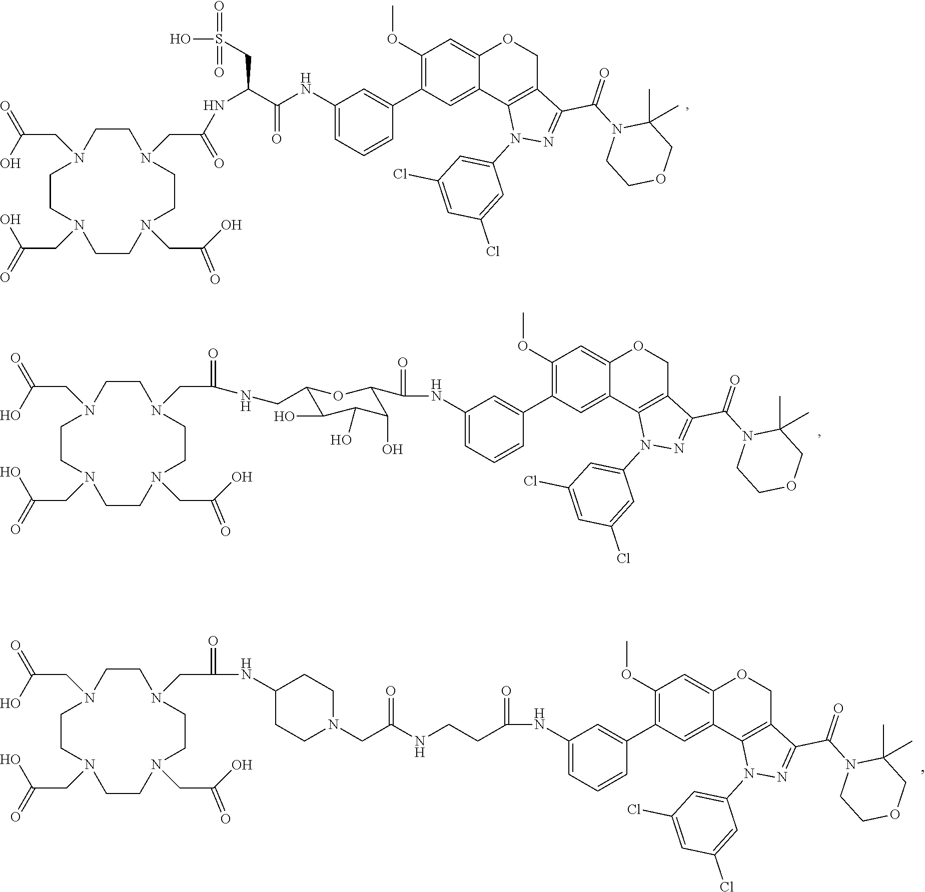 [SMILES: COc1cc2c(cc1-c1cccc(NC(=O)[C@H](CS(=O)(=O)O)NC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2 ; COc1cc2c(cc1-c1cccc(NC(=O)C3OC(CNC(=O)CN4CCN(CC(=O)O)CCN(CC(=O)O)CCN(CC(=O)O)CC4)C(O)C(O)C3O)c1)-c1c(c(C(=O)N3CCOCC3(C)C)nn1-c1cc(Cl)cc(Cl)c1)CO2 ; COc1cc2c(cc1-c1cccc(NC(=O)CCNC(=O)CN3CCC(NC(=O)CN4CCN(CC(=O)O)CCN(CC(=O)O)CCN(CC(=O)O)CC4)CC3)c1)-c1c(c(C(=O)N3CCOCC3(C)C)nn1-c1cc(Cl)cc(Cl)c1)CO2] 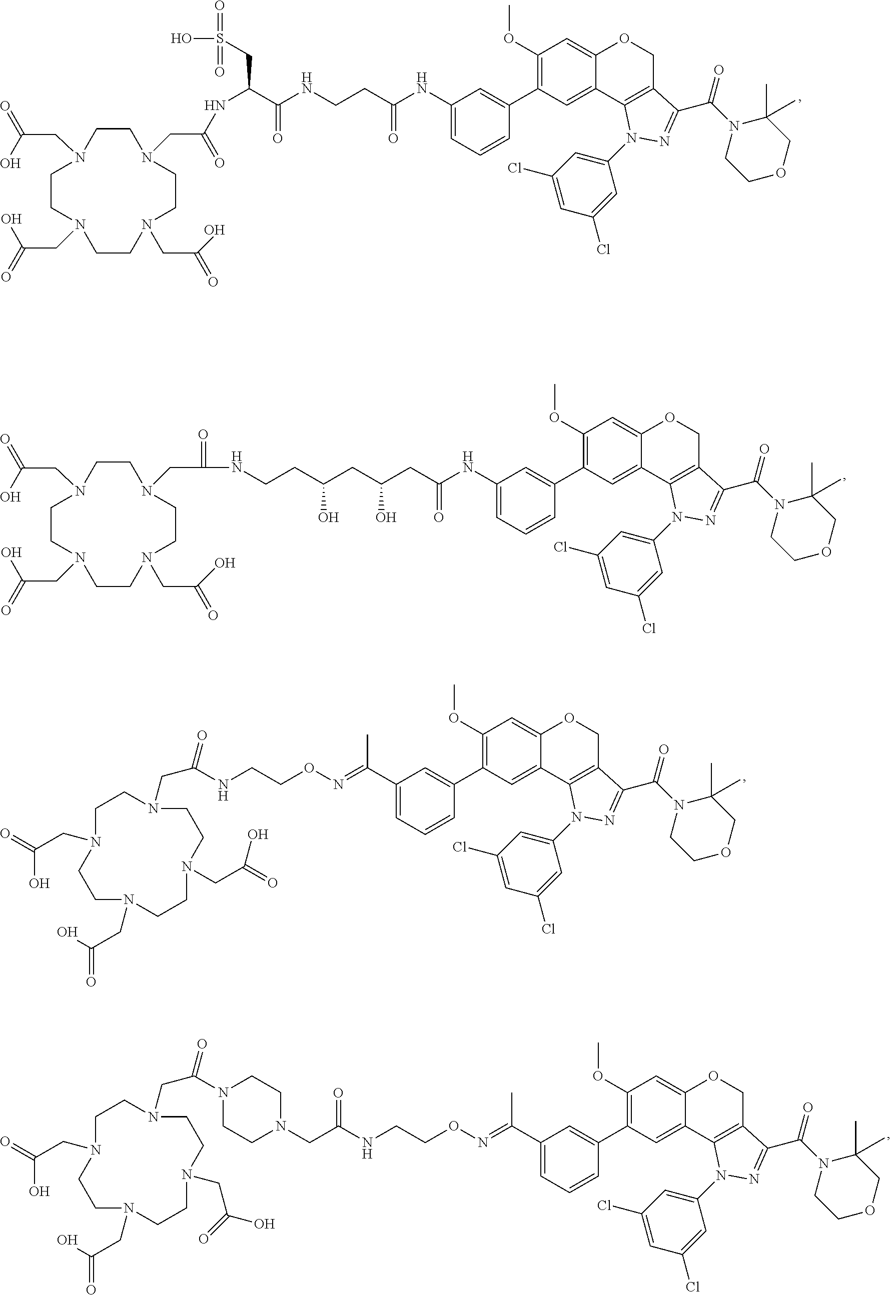 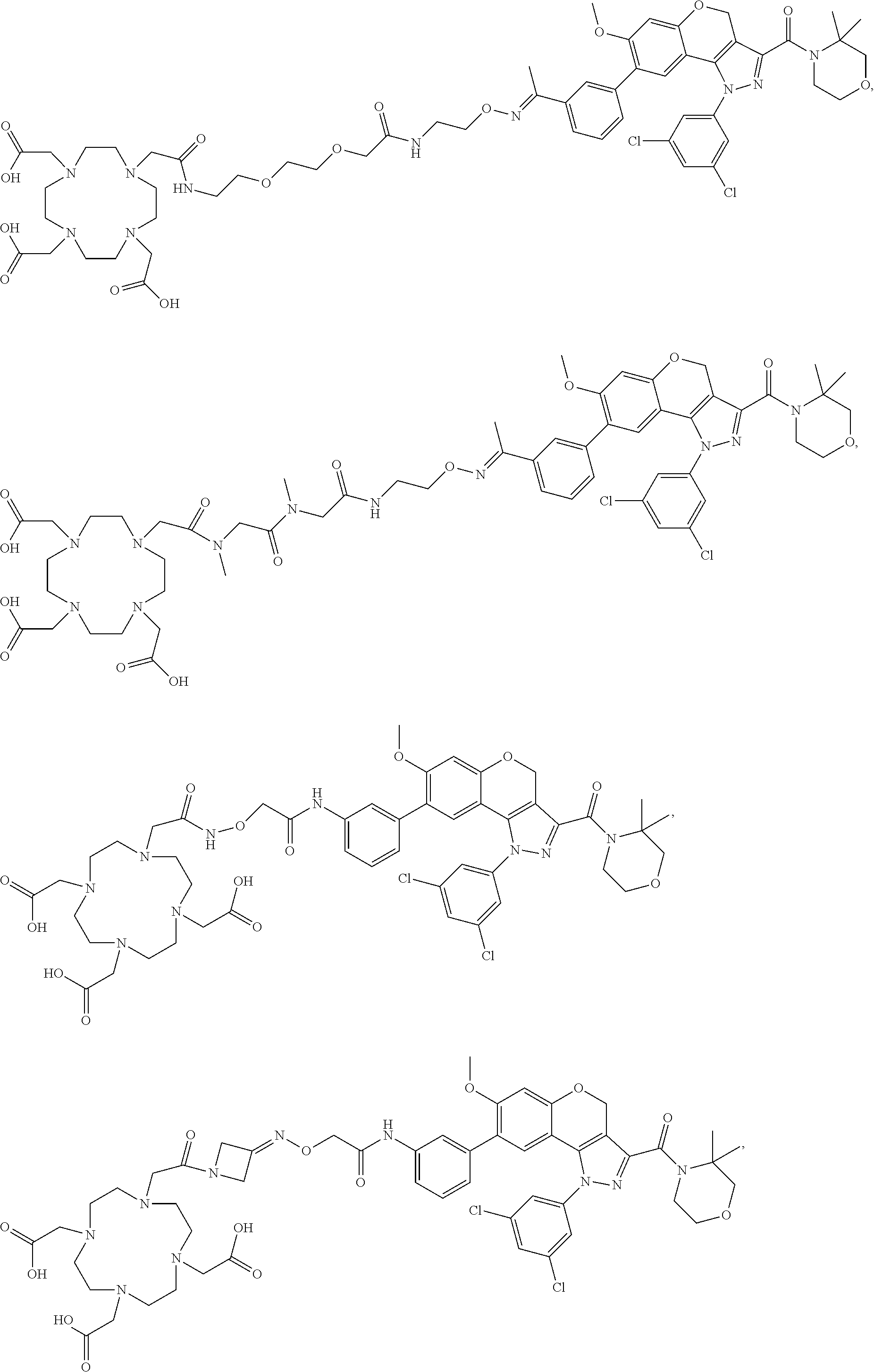 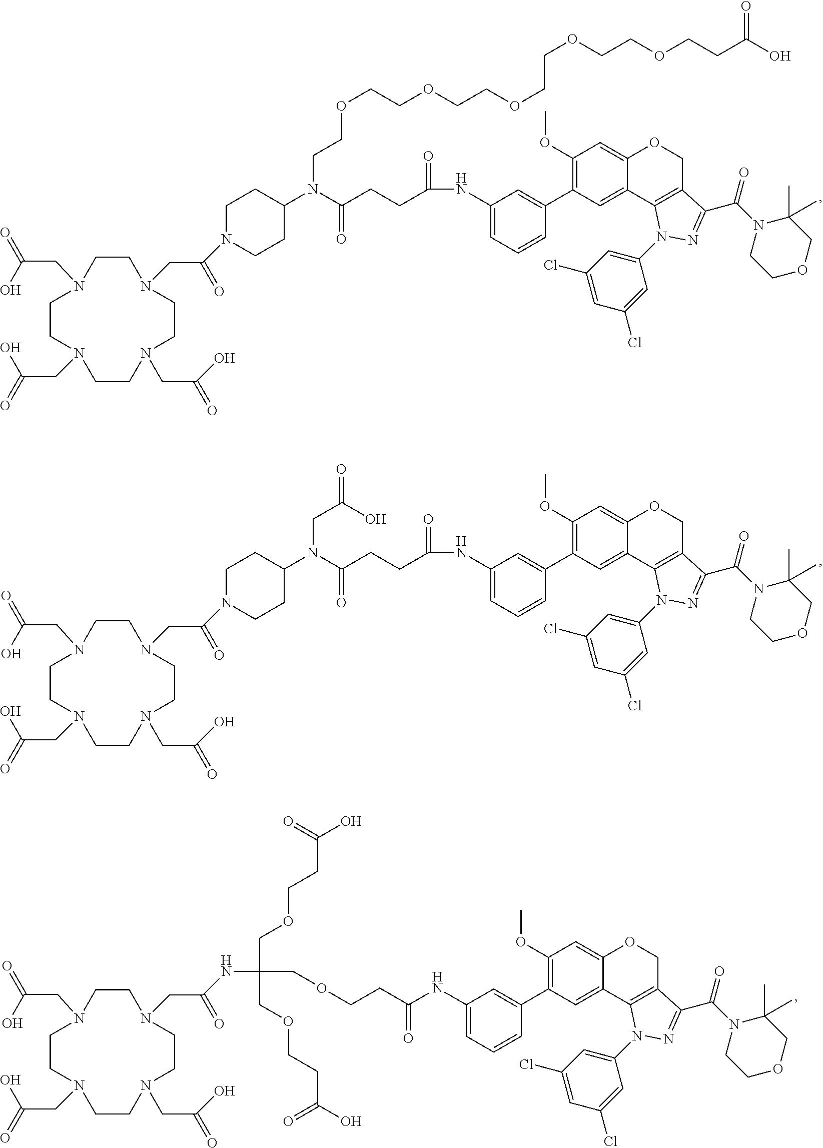 [SMILES: COc1cc2c(cc1-c1cccc(NC(=O)CCC(=O)N(CCOCCOCCOCCOCCOCCC(=O)O)C3CCN(C(=O)CN4CCN(CC(=O)O)CCN(CC(=O)O)CCN(CC(=O)O)CC4)CC3)c1)-c1c(c(C(=O)N3CCOCC3(C)C)nn1-c1cc(Cl)cc(Cl)c1)CO2 ; COc1cc2c(cc1-c1cccc(NC(=O)CCC(=O)N(CC(=O)O)C3CCN(C(=O)CN4CCN(CC(=O)O)CCN(CC(=O)O)CCN(CC(=O)O)CC4)CC3)c1)-c1c(c(C(=O)N3CCOCC3(C)C)nn1-c1cc(Cl)cc(Cl)c1)CO2 ; COc1cc2c(cc1-c1cccc(NC(=O)CCOCC(COCCC(=O)O)(COCCC(=O)O)NC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2] 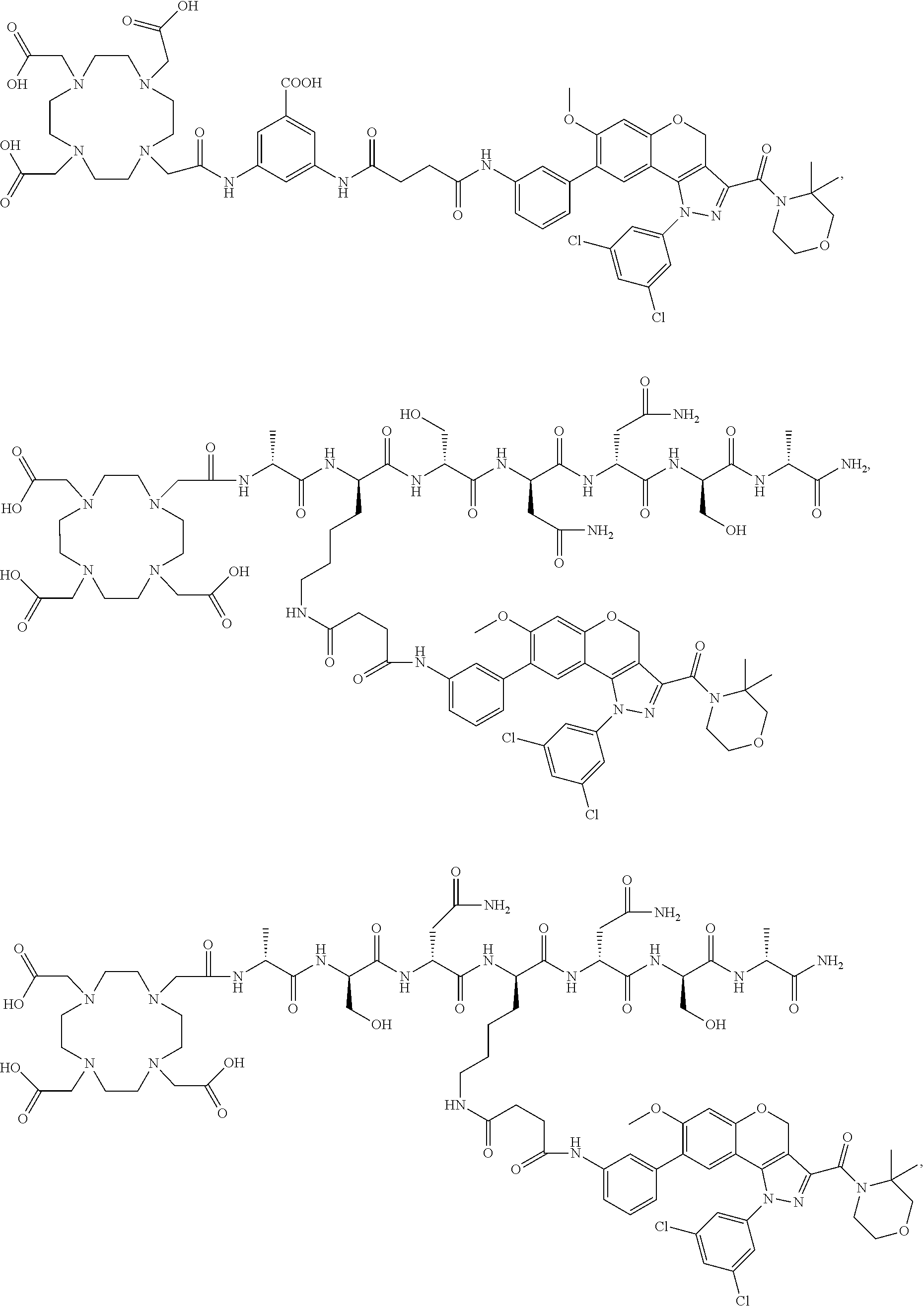 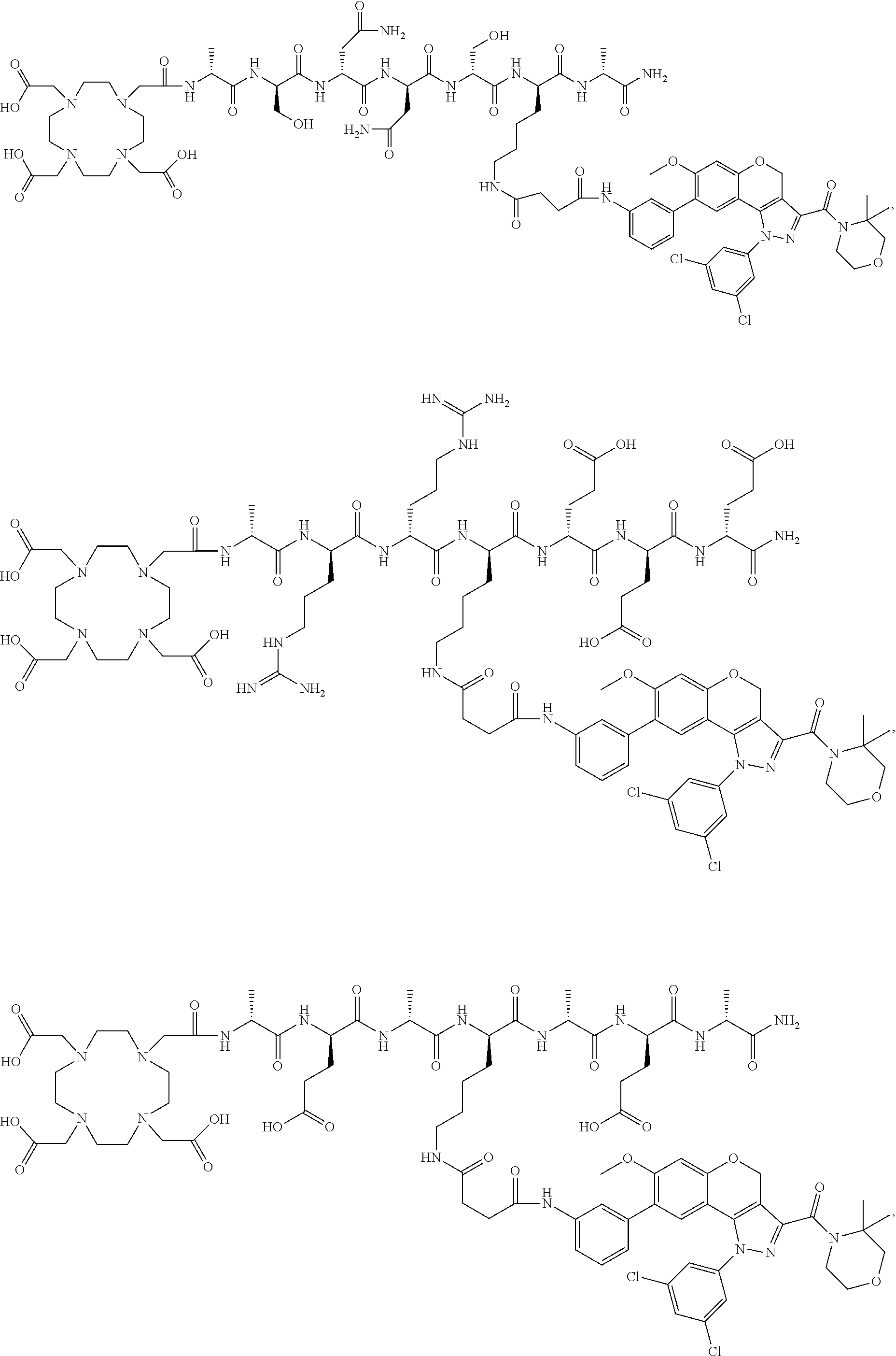 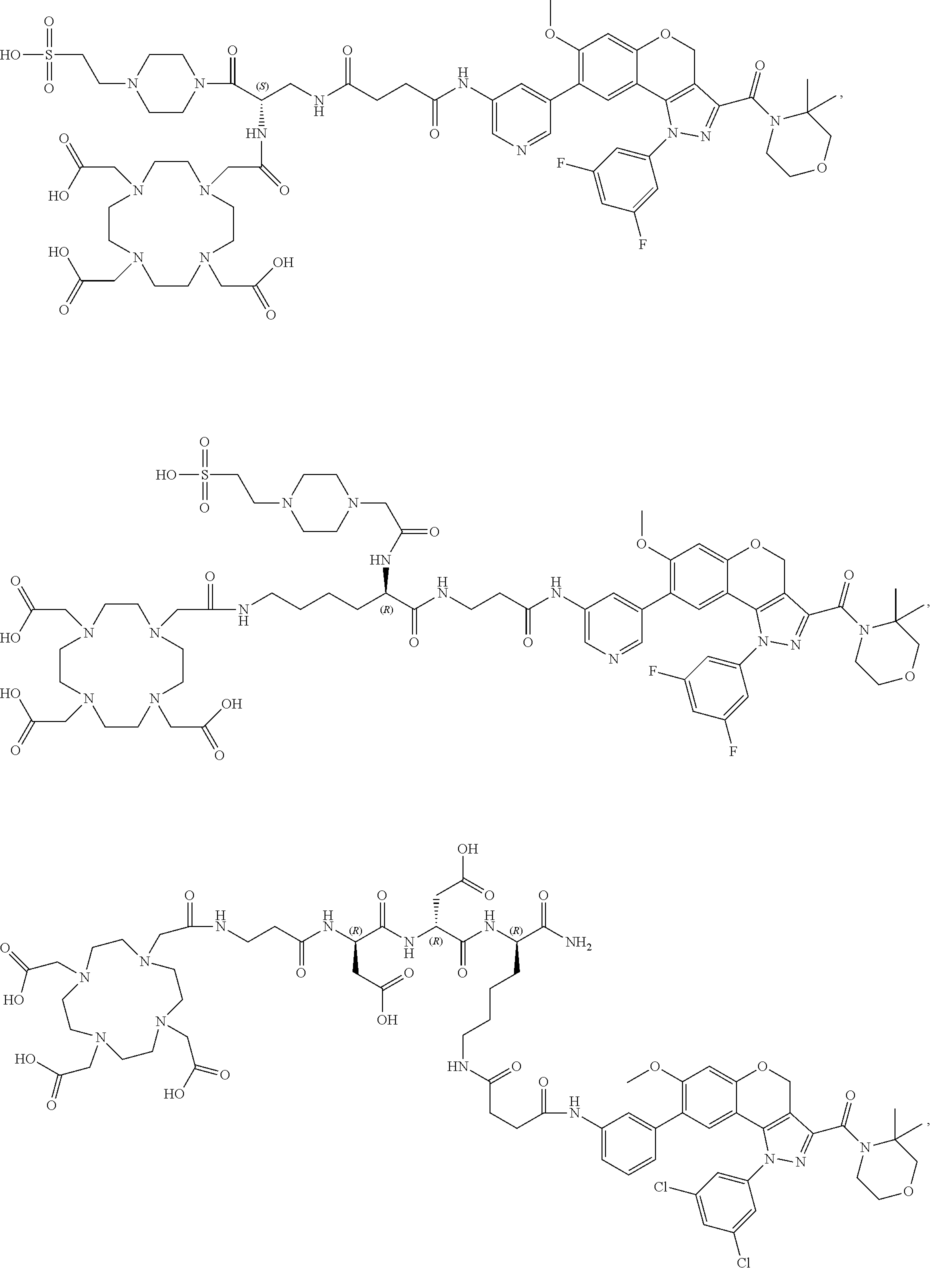 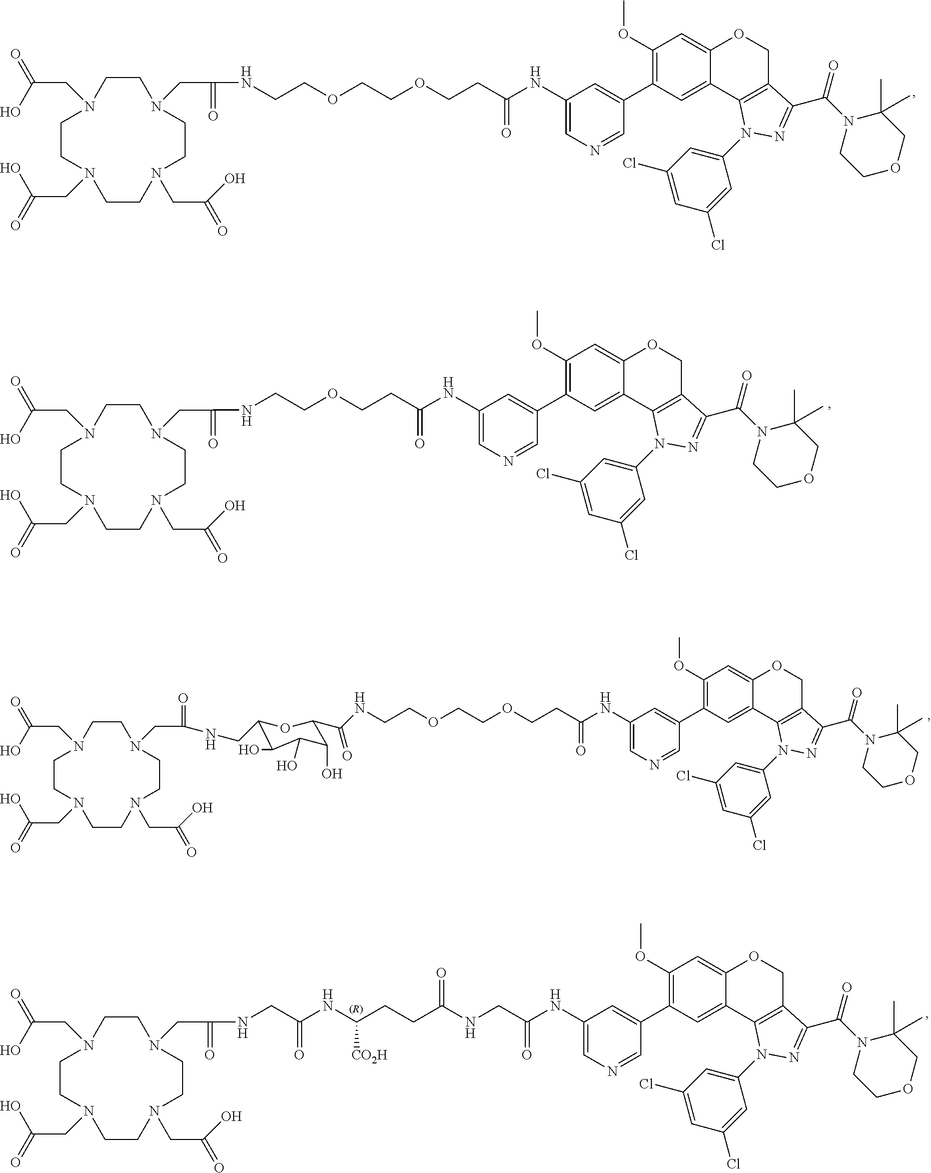 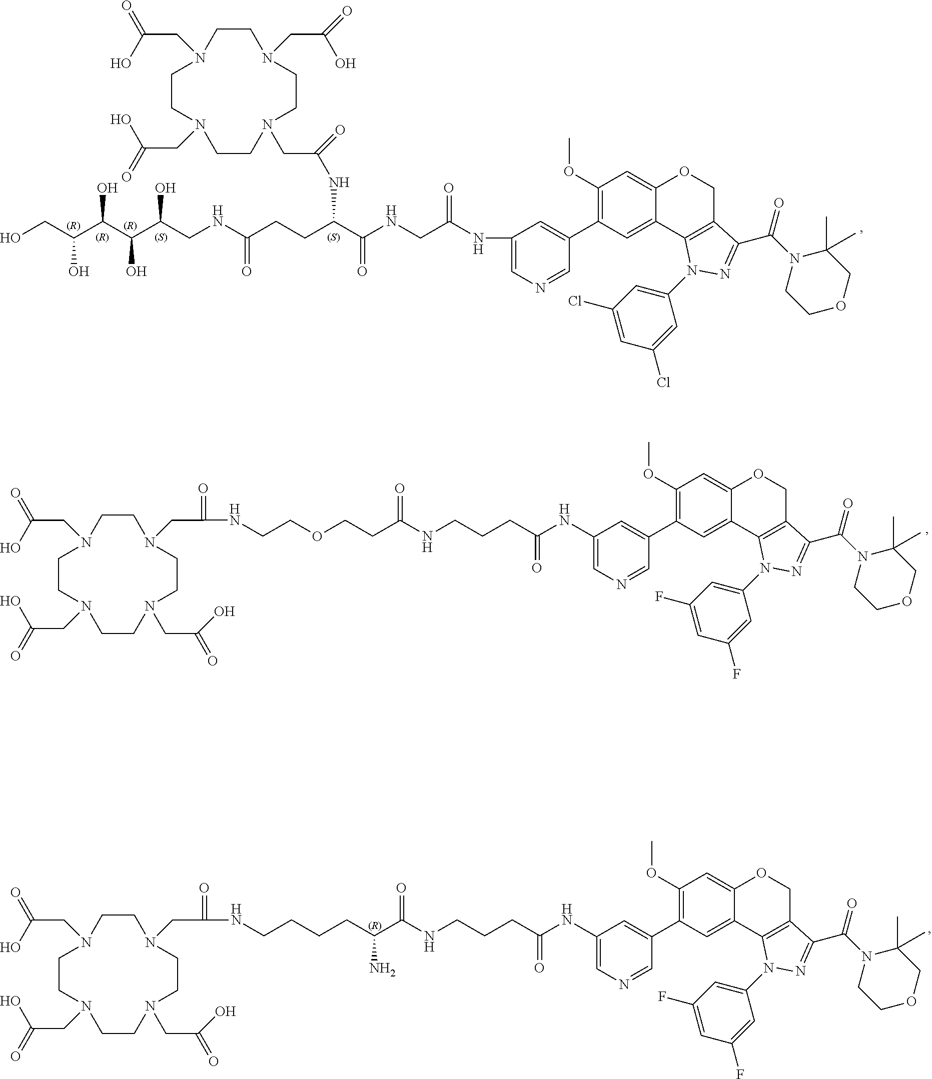 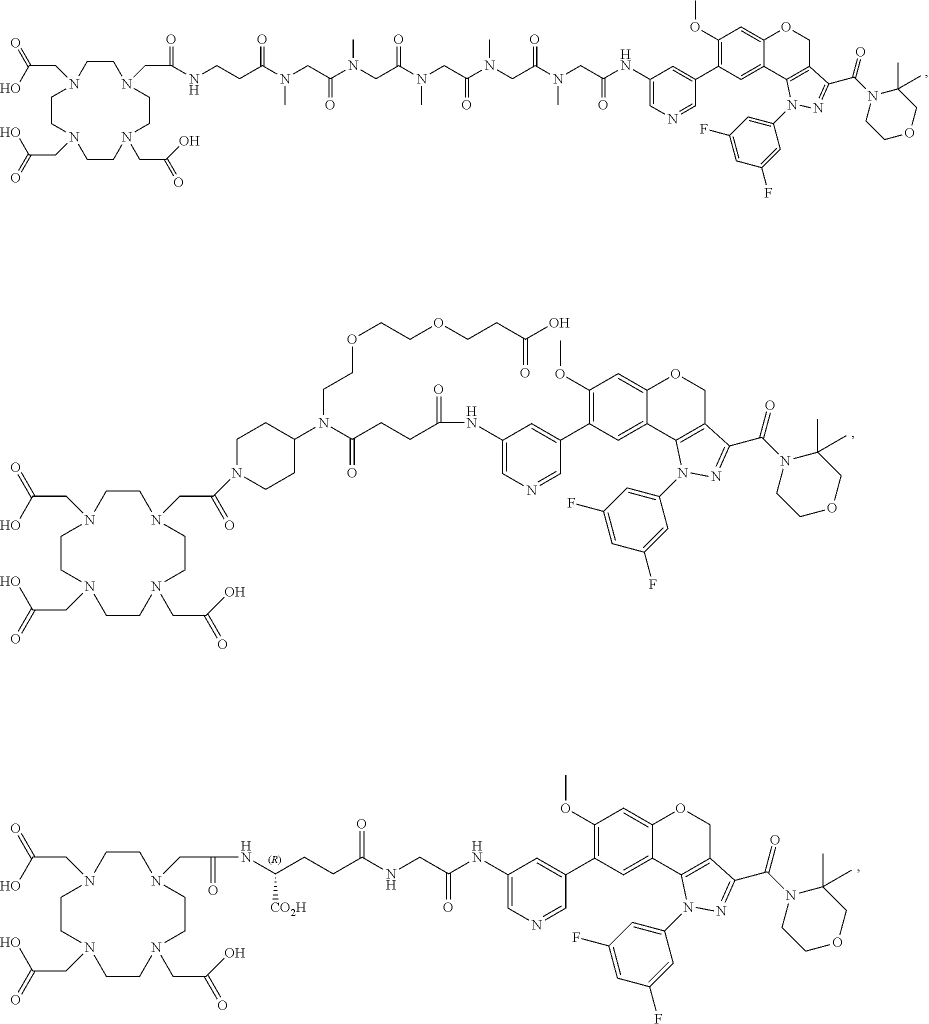 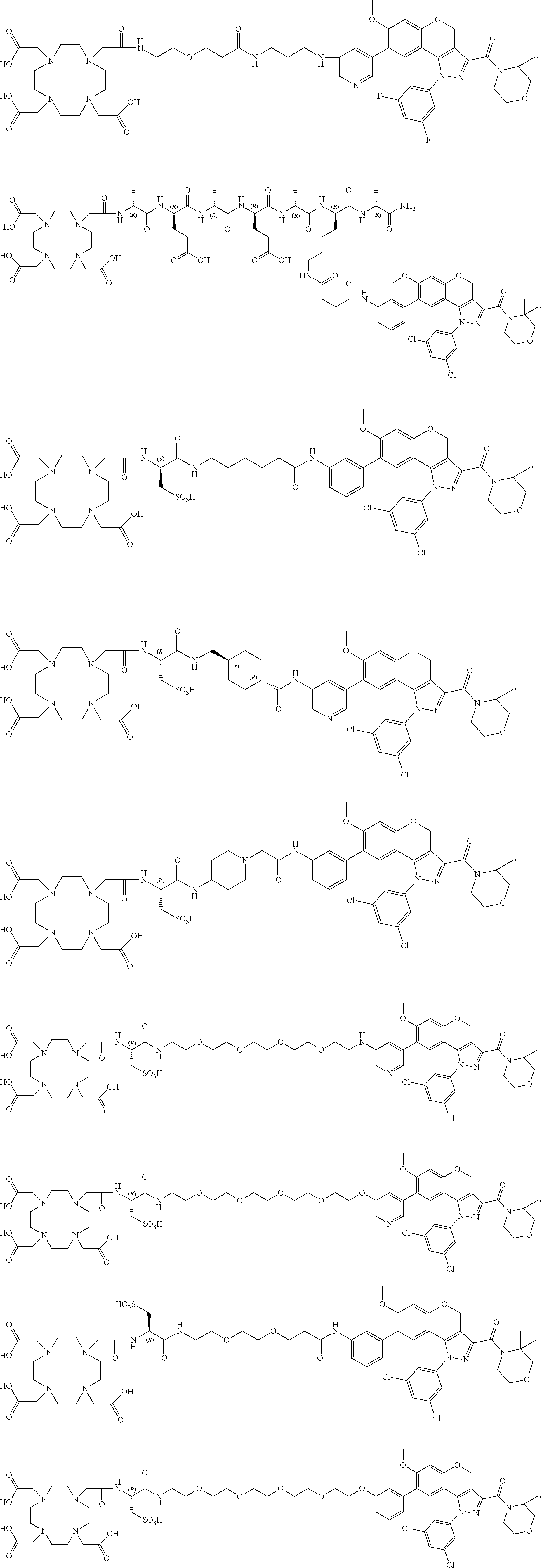 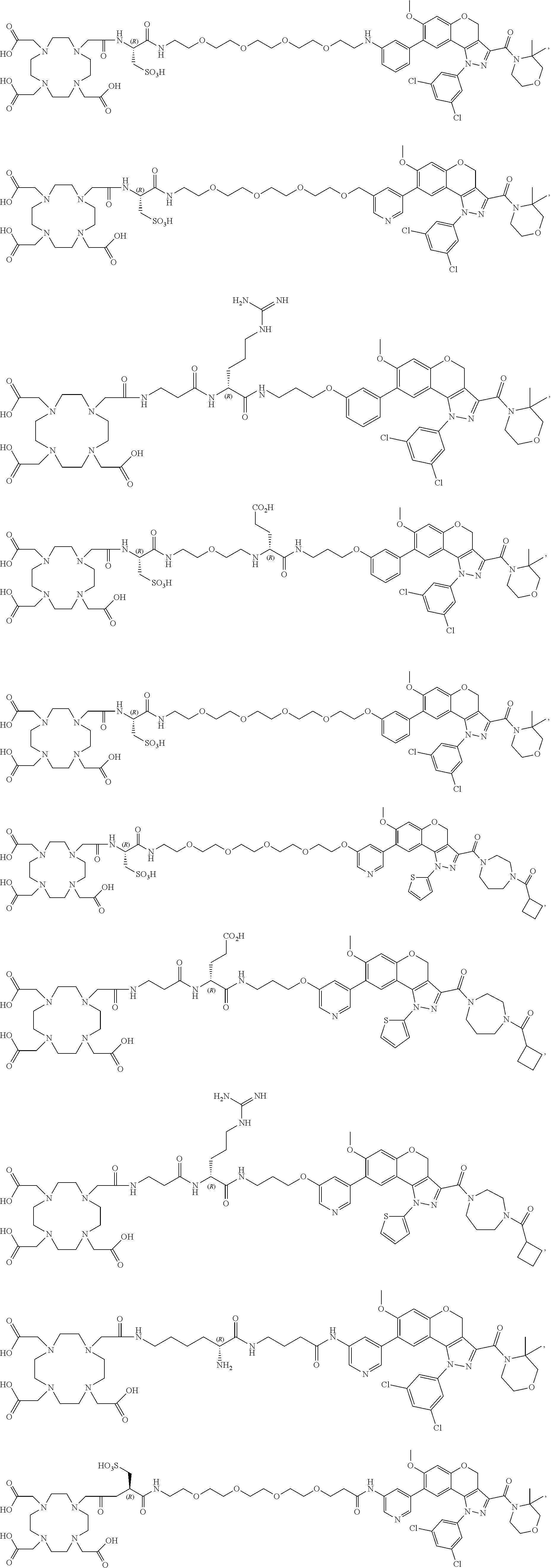 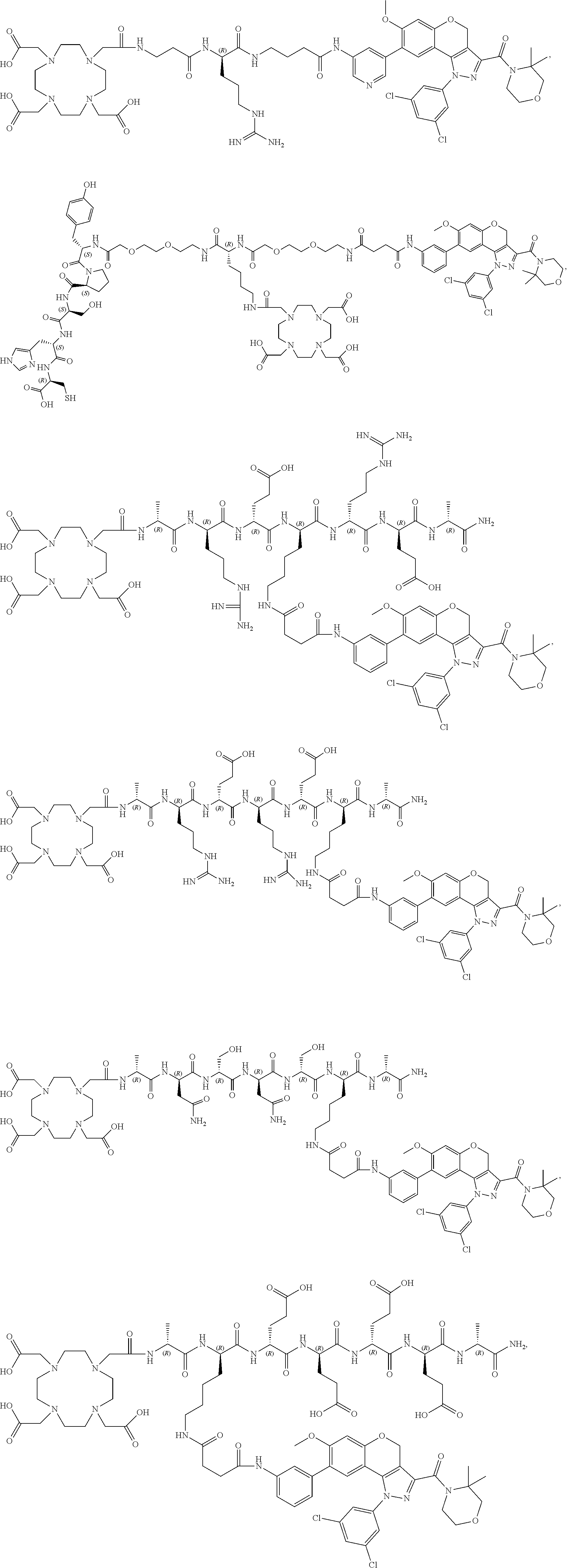 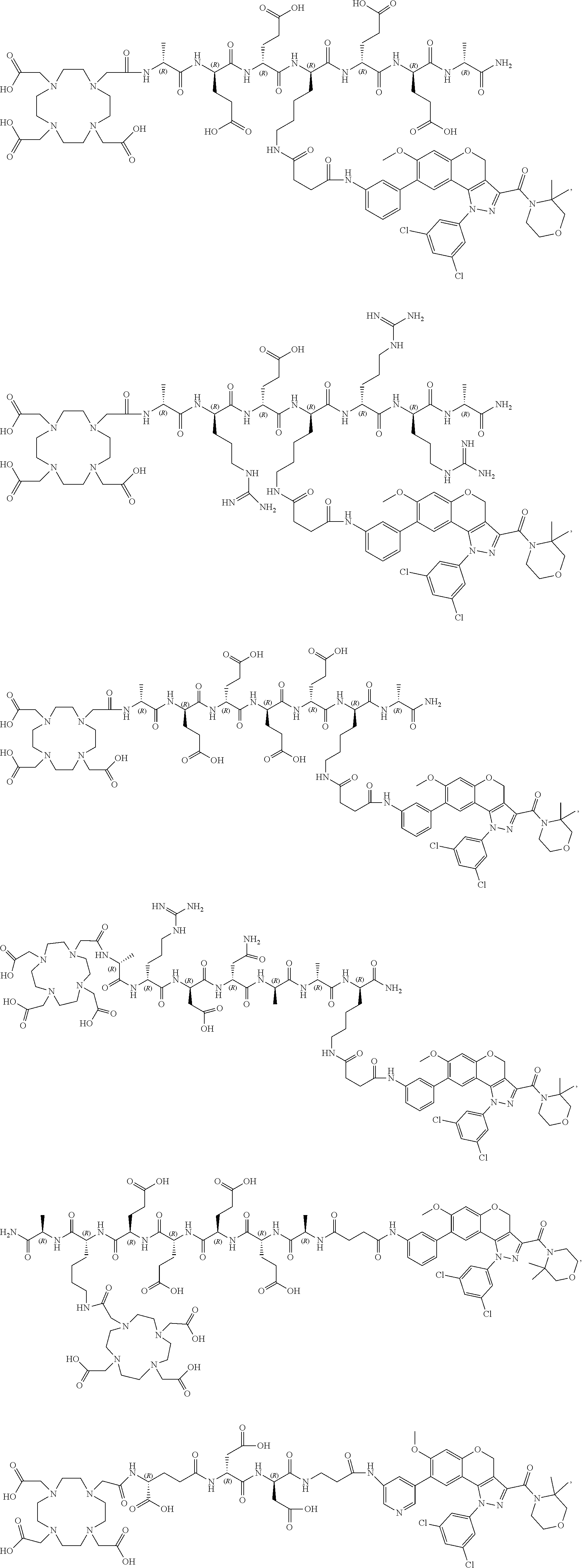 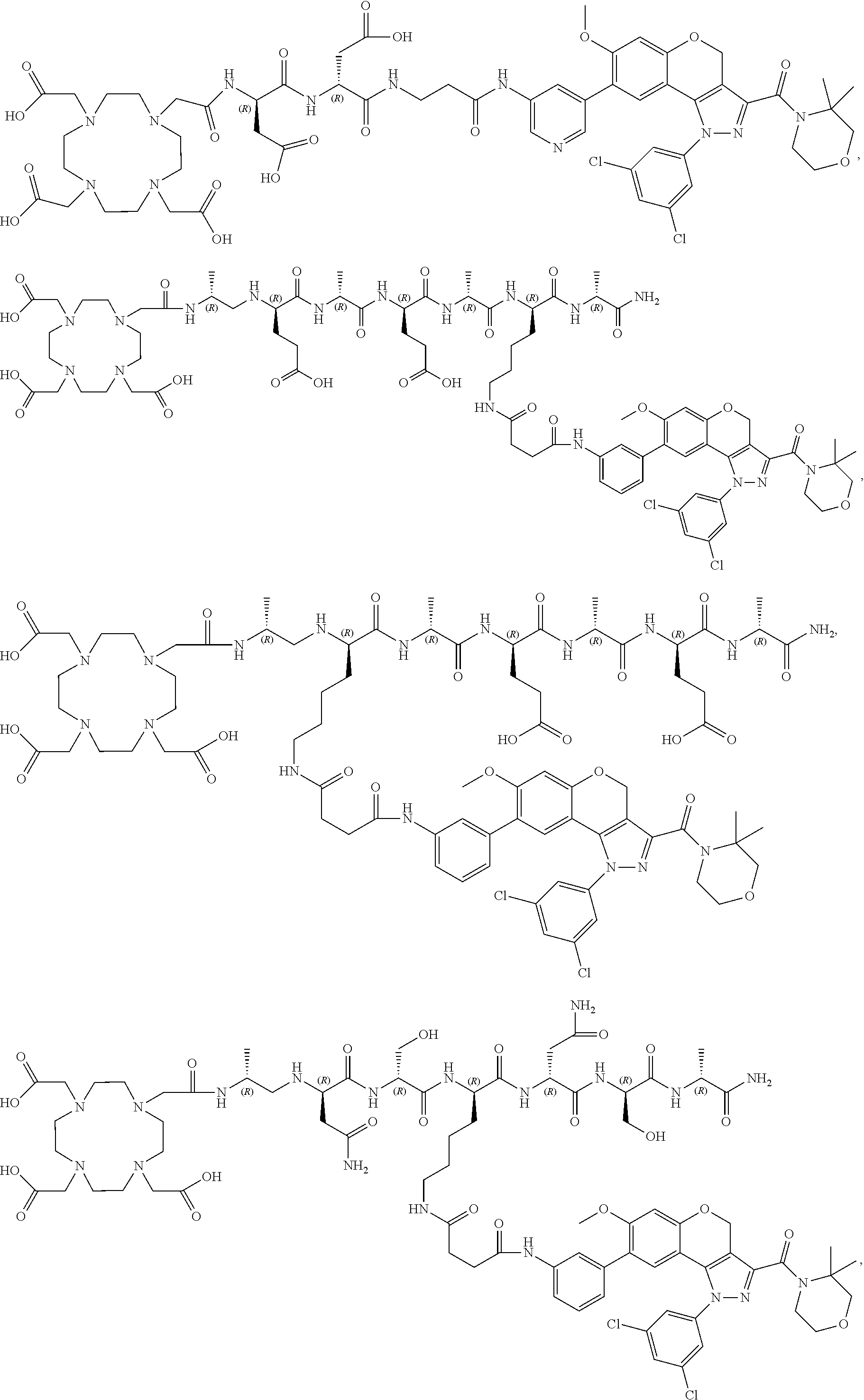 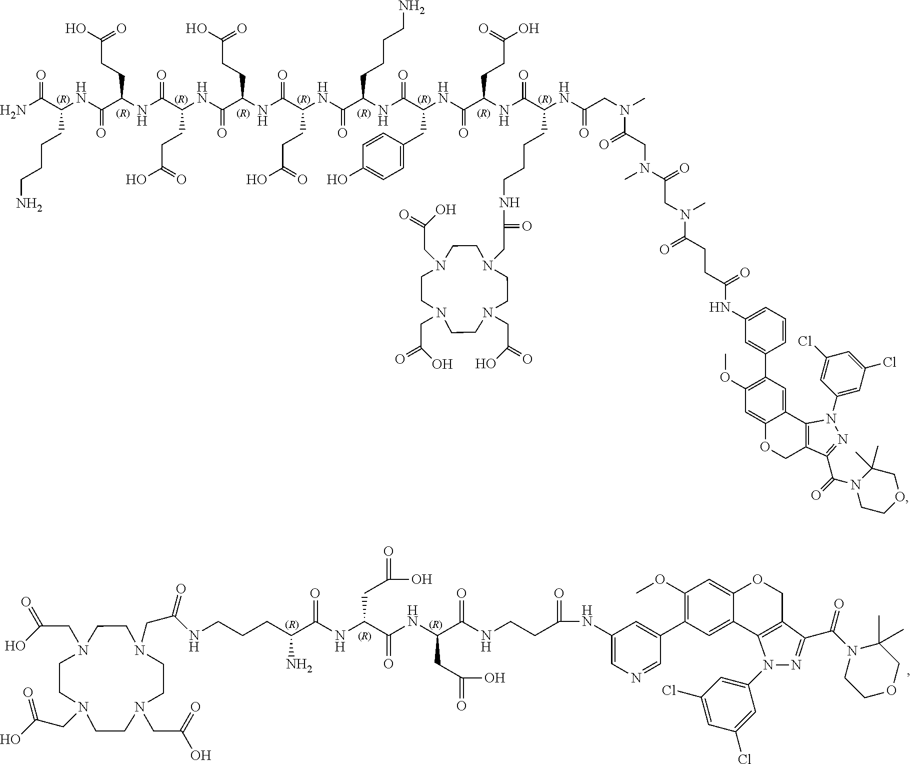
or radionuclide complex thereof.

113. The compound of claim 10, or a pharmaceutically acceptable salt thereof, wherein the compound of Formula (B) has one of the following structures, or a pharmaceutically acceptable salt thereof: 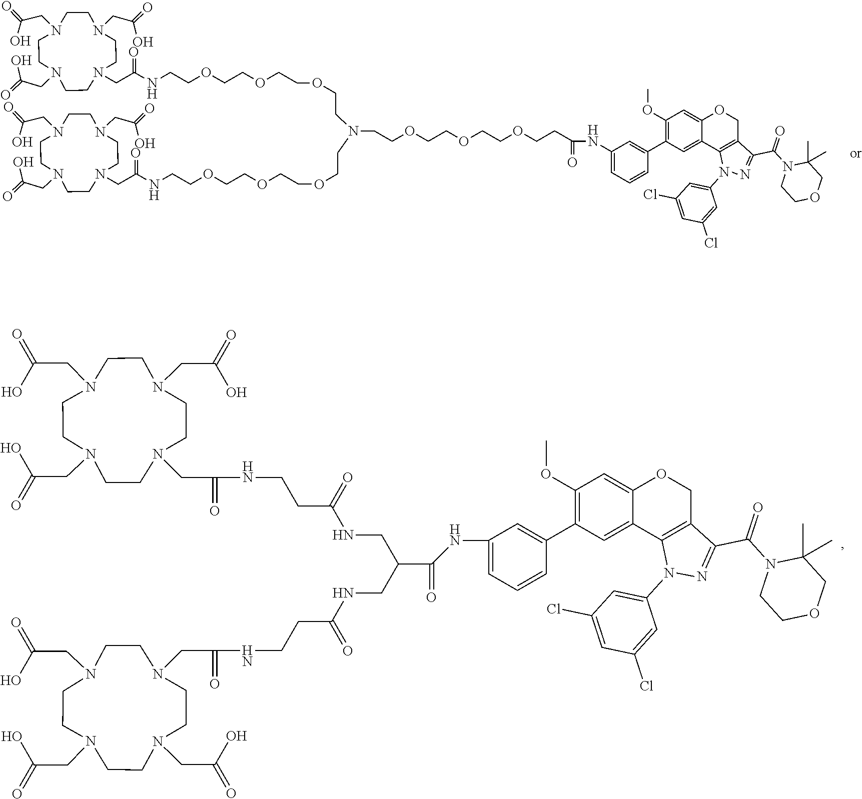 [SMILES: COc1cc2c(cc1-c1cccc(NC(=O)CCOCCOCCOCCN(CCOCCOCCOCCNC(=O)CN3CCN(CC(=O)O)CCN(CC(=O)O)CCN(CC(=O)O)CC3)CCOCCOCCOCCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2 ; COc1cc2c(cc1-c1cccc(NC(=O)C(CNC(=O)CCNC(=O)CN3CCN(CC(=O)O)CCN(CC(=O)O)CCN(CC(=O)O)CC3)CNC(=O)CCNC(=O)CN3CCN(CC(=O)O)CCN(CC(=O)O)CCN(CC(=O)O)CC3)c1)-c1c(c(C(=O)N3CCOCC3(C)C)nn1-c1cc(Cl)cc(Cl)c1)CO2]
or radionuclide complex thereof.

114. The compound of any one of claims 1-113, or a pharmaceutically acceptable salt thereof, wherein: the radionuclide of the radionuclide complex is a lanthanide or an actinide.

115. The compound of any one of claims 1-113, or a pharmaceutically acceptable salt thereof, wherein: the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

116. The compound of any one of claims 1-113, or a pharmaceutically acceptable salt thereof, wherein: the radionuclide of the radionuclide complex is a diagnostic or therapeutic radionuclide.

117. The compound of any one of claims 1-113, or a pharmaceutically acceptable salt thereof, wherein: the radionuclide of the radionuclide complex is an Auger electron-emitting radionuclide, α-emitting radionuclide, β-emitting radionuclide, or γ-emitting radionuclide.

118. The compound of any one of claims 1-113, or a pharmaceutically acceptable salt thereof, wherein the radionuclide of the radionuclide complex is:
an Auger electron-emitting radionuclide that is 111-indium (¹¹¹ In), 67-gallium (⁶⁷Ga), 68-gallium (⁶⁸Ga), 99m-technetium (^{99m}Tc), or 195m-platinum (^{195m}Pt); or
an α-emitting radionuclide that is 225-actinium (²²⁵ Ac), 213-bismuth (²¹³Bi), 223-Radium (²²³Ra), or 212-lead (²¹²Pb); or
a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (89Sr), 198-gold (198 Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or
a γ-emitting radionuclide that is 60-cobalt (⁶⁰Co), 103-palldium (¹⁰³Pd), 137-cesium (¹³⁷Cs), 169-ytterbium (¹⁶⁹Yb), 192-iridium (¹⁹²Ir), or 226-radium (²²⁶Ra).

119. The compound of any one of claims 1-113, or a pharmaceutically acceptable salt thereof, wherein: the radionuclide of the radionuclide complex is 111-indium (¹¹¹ In), 115-indium (¹¹⁵ In), 67-gallium (⁶⁷Ga), 68-gallium (⁶⁸Ga), 70-gallium (⁷⁰Ga), 225-actinium (225 Ac), 175-lutetium (¹⁷⁵Lu) or 177-lutetium (¹⁷⁷Lu).

120. A pharmaceutical composition comprising a compound of any one of claims 1-119, or a pharmaceutically acceptable salt thereof, and at least one pharmaceutically acceptable excipient.

121. The pharmaceutical composition of claim 120, wherein the pharmaceutical composition is formulated for administration to a mammal by intravenous administration.

122. A method for the treatment of cancer comprising administering to a mammal with cancer an effective amount of a compound of any one of claims 1-119, or a pharmaceutically acceptable salt thereof.

123. The method of claim 122, wherein the cancer comprises tumors and the tumor overexpress the follicle-stimulating hormone receptor (FSHR).

124. The method of claim 122 or claim 123, wherein the cancer is ovarian cancer, prostate cancer, breast cancer, testicular cancer, lung cancer, liver adenocarcinoma, colon adenocarcinoma, stomach adenocarcinoma, kidney cancer or bladder cancer.

125. The method of claim 122 or claim 123, wherein the cancer is ovarian cancer.

126. The method of claim 122 or claim 123, wherein the cancer is prostate cancer.

127. The method of claim 122 or claim 123, wherein the cancer is testicular cancer.

128. A method of killing tumors in a mammal that overexpress the follicle-stimulating hormone receptor (FSHR) comprising administering to the mammal a compound of any one of claims 1-119, or a pharmaceutically acceptable salt thereof, wherein the compound of any one of claims 1-119, or a pharmaceutically acceptable salt thereof, comprises a therapeutic radionuclide.

129. The method of claim 128, wherein the mammal has been diagnosed with ovarian cancer, prostate cancer, breast cancer, testicular cancer, lung cancer, liver adenocarcinoma, colon adenocarcinoma, stomach adenocarcinoma, kidney cancer or bladder cancer.

130. The method of claim 128, wherein the mammal has been diagnosed with ovarian cancer.

131. The method of claim 128, wherein the mammal has been diagnosed with prostate cancer.

132. The method of claim 128, wherein the mammal has been diagnosed with testicular cancer.

133. A method for identifying tumors expressing the follicle-stimulating hormone receptor (FSHR) in a mammal comprising administering to the mammal a compound of any one of claims 1-119, or a pharmaceutically acceptable salt thereof; and performing positron emission tomography (PET) analysis, single-photon emission computerized tomography (SPECT), or magnetic resonance imaging (MRI); wherein the compound of any one of claims 1-119, or a pharmaceutically acceptable salt thereof, comprises a diagnostic radionuclide.

134. A method for the in vivo imaging of tissues or organs in a mammal with tumors expressing the follicle-stimulating hormone receptor (FSHR) comprising administering to the mammal a compound of any one of claims 1-119, or a pharmaceutically acceptable salt thereof, and performing positron emission tomography (PET) analysis, single-photon emission computerized tomography (SPECT), or magnetic resonance imaging (MRI); wherein the compound of any one of claims 1-119, or a pharmaceutically acceptable salt thereof, comprises a diagnostic radionuclide.
